(19) United States
(12) Patent Application Publication
(10) Pub. No.: US 2026/0193180 A1
(43) Pub. Date: Jul. 9, 2026

(54) AUTOPHAGY INDUCING COMPOUNDS AND USES THEREOF

(71) Applicant: SAMSARA THERAPEUTICS INC., Lewes, DE (US)

(72) Inventors: Peter Hamley, Oxford (GB); Warren Galloway, Kronberg Im Taunus (DE); Ben Atkinson, Wallingford (GB)

(21) Appl. No.: 19/129,855

(22) PCT Filed: Nov. 14, 2023
(86) PCT No.: PCT/US2023/037259

§ 371 Date: May 14, 2025

(30) Foreign Application Priority Data
Nov. 15, 2022 (EP) 22207631.7

(51) Int. Cl. C07D 205/04; A61K 31/397; A61K 31/402; A61K 31/4025; A61K 31/4468; A61K 31/495; A61K 31/5375; A61P 21/00; A61P 25/16; A61P 25/28; C07D 207/09; C07D 207/14; C07D 211/26; C07D 211/58; C07D 241/04; C07D 265/30; C07D 295/096; C07D 405/06

(52) U.S. Cl. CPC C07D 205/04; A61K 31/397; A61K 31/402; A61K 31/4025; A61K 31/4468; A61K 31/495; A61K 31/5375; A61P 21/00; A61P 25/16; A61P 25/28; C07D 207/09; C07D 207/14; C07D 211/26; C07D 211/58; C07D 241/04; C07D 265/30; C07D 295/096; C07D 405/06

(57) ABSTRACT

The present invention relates to compounds of formula (I) or a salt, stereoisomer, tautomer or N-oxide thereof. Furthermore, the present invention relates to compounds of formula (I), or a salt, stereoisomer, tautomer or N-oxide thereof, which are suitable for inducing and/or stimulating the process of autophagy, as well as compounds of formula (I) for use in medicine and the treatment of autophagy related diseases or disorders.

### FIELD OF THE INVENTION

The present invention relates to compounds of formula (I) or a salt, stereoisomer, tautomer or N-oxide thereof. Furthermore, the present invention relates to compounds of formula (I), or a salt, stereoisomer, tautomer or N-oxide thereof, which are suitable for inducing and/or stimulating the process of autophagy, as well as the compounds of formula (I) for use in medicine and for use in the treatment of autophagy-related diseases or conditions.

### BACKGROUND OF THE INVENTION

Autophagy is the cell's method for degradation of unnecessary or damaged components such as proteins, organelles and invading pathogens. It utilises an orchestrated process resulting in lysosomal degradation resulting in recycling of cellular components and maintenance of cellular homeostasis.

The cell-autonomous antimicrobial defense functions of autophagy, demonstrated initially in the case of streptococci and Mycobacterium tuberculosis have been extended to a wide variety of microbes with a caveat that most highly adapted pathogens have evolved specific protective mechanisms against autophagic elimination of microbes. Other studies have uncovered orderly intersections between autophagy and innate and adaptive immunity, T cell development, differentiation and homeostasis, and inflammatory responses. Thus, autophagy plays a large role in various diseases such as cancer, inflammatory disease, degenerative neurological disease, and immune disease. Autophagy is a cell survival mechanism that is induced in stressed cells.

Towers and Thorburn (Therapeutic Targeting of Autophagy. EBioMedicine. 2016; 14:15-23) disclose that autophagy is widely accepted as cytoprotective against neurodegenerative diseases and a variety of clinical interventions are moving forward to increase autophagy as a therapeutic intervention. Autophagy has both positive and negative roles in cancer and this has led to controversy over whether or how autophagy manipulation should be attempted in cancer therapy. Nevertheless, cancer is the disease where most current activity in trying to manipulate autophagy for therapy is taking place and dozens of clinical trials are using autophagy inhibition with Chloroquine or Hydroxychloroquine in combination with other drugs for the treatment of multiple neoplasms. They review recent literature implicating autophagy in neurodegenerative diseases and cancer and highlight some of the opportunities, controversies and potential pitfalls of therapeutically targeting autophagy.

Mulcahy Levy and Thorburn (Autophagy in cancer: moving from understanding mechanism to improving therapy responses in patients. Cell Death Differ 27, 543-857 (2020)) disclose that autophagy allows for cellular material to be delivered to lysosomes for degradation resulting in basal or stress-induced turnover of cell components that provide energy and macromolecular precursors. These activities are thought to be particularly important in cancer where both tumor-promoting and tumor-inhibiting functions of autophagy have been described. Autophagy has also been intricately linked to apoptosis and programmed cell death, and understanding these interactions is becoming increasingly important in improving cancer therapy and patient outcomes. In the review, they consider how recent discoveries about how autophagy manipulation elicits its effects on cancer cell behavior can be leveraged to improve therapeutic responses.

Grainger et al. (1995, Nature Medicine 1: 1067-1073) and Reckless et al. (1997, Circulation 95: 1542-1548) have demonstrated that tamoxifen, a potent inducer of autophagy, inhibited atherosclerosis in mice models by suppressing the diet-induced formation of lipid lesions in the aorta by lowering of low-density lipoprotein (LDL) cholesterol.

Dikic and Elazar (Mechanism and medical implications of mammalian autophagy. Nat Rev Mol Cell Biol 2018 19(6):349-364) discuss that autophagy is deregulated in the context of various human pathologies, including cancer and neurodegeneration, and its modulation has considerable potential as a therapeutic approach.

Pierzynowska et al. (Metab Brain Dis 2018; 33(4):989-1008) focuses on ways by which autophagy can be stimulated and discusses that activation of autophagy by different factors or processes can be considered as a therapeutic strategy in metabolic neurodegenerative diseases.

The development of more selective autophagy inducers is needed for the treatment and/or prevention of diseases where autophagy plays a role. It is an object of the present invention to provide effective agents that can be used for the prevention and treatment of conditions and diseases that can be treated/prevented by inducing and/or stimulating autophagy, in particular cancer, age-related diseases, and infections.

### OBJECTS AND SUMMARY OF THE INVENTION

It is therefore an object of the present invention to provide compounds, which induce and/or stimulate autophagy. It is another object of the present invention to provide compounds, which are suitable for use as a medicament. It is another object of the present invention to provide compounds, which are suitable for use in the treatment of an autophagy-related disease or condition, in particular cancer, age-related diseases, and infections. It is yet another object of the present invention to provide compounds, which are suitable for use in the treatment of one or more autophagy-related diseases selected from the group consisting of neurodegenerative diseases, Huntington's disease, Alzheimer's disease, Parkinson's disease, systemic lupus erythematosus, epilepsy, cancer, liver diseases (e.g. nonalcoholic fatty liver disease (NAFLD)), al antitrypsin deficiency, Charcot Marie Tooth syndrome, Rett Syndrome, Sickle Cell disease, Wilson Disease, amyloidosis, Gaucher's diseases, lysosomal and glycogen storage disorders (e.g., Glycogen Storage Disease type 1A (GSD1A)), cystic fibrosis; viral infection and diseases human cytomegalovirus (HCMV) infection, hepatitis B, human immunodeficiency virus infection, Zika virus infection, coronavirus infection, HCoV-229E, HCoV-NL63, betacoronavirus infection, such as HCoV-OC43, SARS-CoV-1, HCoV-HKU1, MERS-CoV or SARS-CoV-2, bacterial infections, metabolic disorders, diabetes, fibrosis, silicosis, diabetic retinopathy, glaucoma, cataracts, age-related macular degeneration, glomerulonephritis, glomerulosclerosis, wound healing disorders, Niemann-Pick type C (NPC) disease, fibrinogen storage disease (FSB), inclusion body disease (IBD), muscular dystrophy, Duchenne muscular dystrophy, Limb-girdle muscular dystrophy, myopathy, myofibrillar myopathy, hereditary myopathy, diabetic cardiomyopathy, anti-inflammatory disorders, autoimmune diseases, multiple sclerosis, rheumatoid arthritis, irritable bowel syndrome, Crohn's disease, vascular disorders, stroke, coronary artery diseases, myocardial infarction, unstable angina pectoris, atherosclerosis or vasculitis, Behcet's syndrome, giant cell arteritis, polymyalgia rheumatic, Wegener's granulomatosis, Churg-Strauss syndrome, vasculitis, Henoch-Schonlein pruprua, Kawasaki disease, viral infection or replication, pox virus infection, herpes virus infection, asthma, allergic rhinitis, COPD, osteoporosis, organ transplant rejection, psoriasis, hypertrophic scarring (keloid formation), adhesion formations following general or gynecological surgery, lung fibrosis, liver fibrosis, kidney fibrosis, disorders caused by intracellular parasites, malaria, tuberculosis, neuropathic pain, post-operative phantom limb pain or postherpetic neuralgia, allergies, amyotrophic lateral sclerosis (ALS), antigen induced recall response, immune response suppression, muscle degeneration and atrophy, frailty in aging, spinal cord injury, and diseases and conditions involving misfolded and/or nonfolded proteins. It is yet a further object of the present invention to provide compounds suitable for use for stimulating autophagy in an in-vitro assay.

The above objects can be achieved by the compounds of formula (I) as defined herein as well as pharmaceutical compositions comprising the same, and by the medical uses thereof. The inventors of the present invention surprisingly found that the compounds of formula (I) as defined herein induce and/or stimulate autophagy. Accordingly, the compounds of formula (I) are for use in medicine, in particular for use in the treatment of an autophagy-related disease or condition, in particular cancer, age-related diseases, and infections.

In a first aspect, the present invention therefore relates to a compound of formula (I)

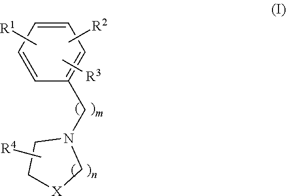

or a salt, stereoisomer, tautomer or N-oxide thereof,
wherein
X is CH₂, CHR⁴, NR^{N} or O;
R¹ is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X};
R² is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X};
R³ is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X}; or
R¹ and R² together with the atoms to which they are attached form a 5- or 6-membered partially unsaturated heterocyclyl, wherein the aforementioned heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{X};
R⁴ is H, C₁-C₄-alkyl, or —(CH₂)ₚ—NR^{a}R^{b};
wherein
R^{N} is H, or C₁-C₄-alkyl;
R^{X} is C₁-C₄-alkyl, C₁-C₄-haloalkyl, NR^{a}R^{b}, or two R^{X} form cyclopropyl;
R^{a}, R^{b} are independently of each other selected from H, and C₁-C₄-alkyl;
R^{c} is H, or C₁-C₄-alkyl;
m is 1, 2, or 3;
n is 0, 1, or 2;
p is 0, 1, 2, or 3.

In a preferred embodiment, the compound is not

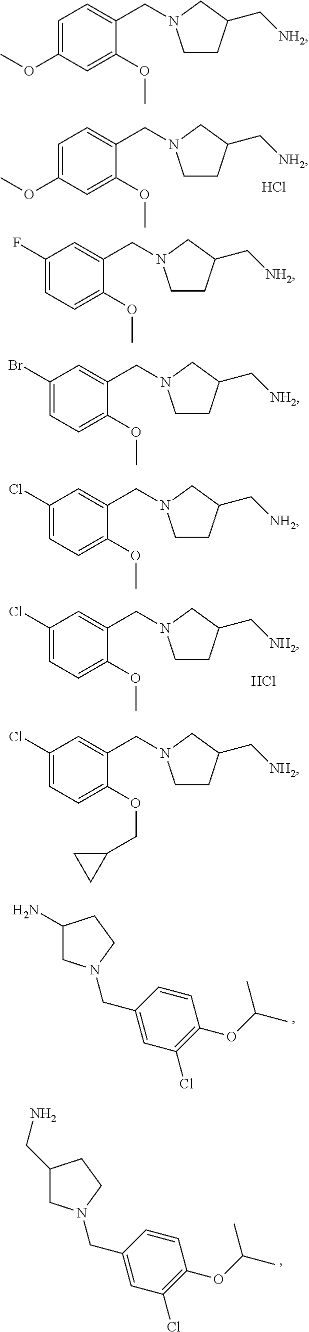

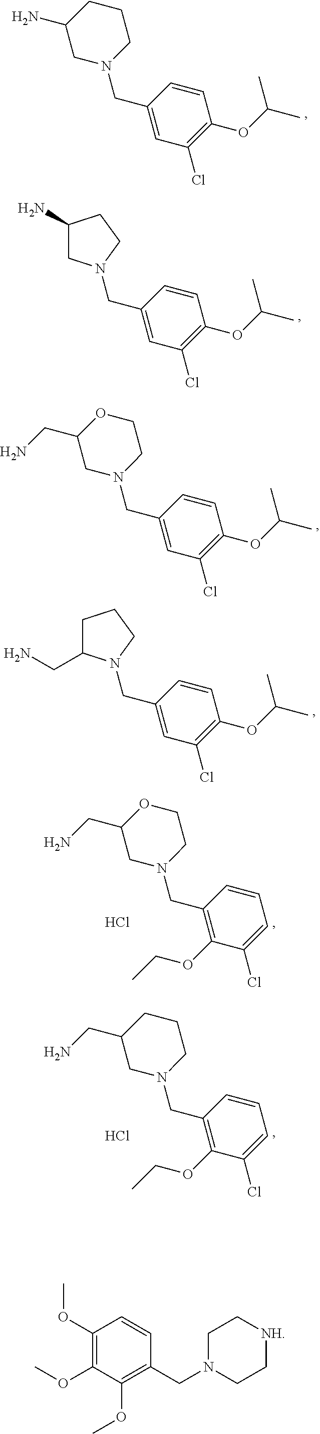

In another preferred embodiment,
R¹ is C₁-C₄-alkoxy, wherein each substitutable carbon atom in the aforementioned groups is independently unsubstituted or substituted with one or more, same or different substituents R^{X};
R² is halogen;
R³ is H or halogen; and
R⁴ is —(CH₂)ₚ—NR^{a}R^{b}.

In another preferred embodiment,
R¹ is C₂-C₄-alkoxy, wherein each substitutable carbon atom in the aforementioned groups is independently unsubstituted or substituted with one or more, same or different substituents R^{X};
R² is Cl;
R³ is H or Cl; and
R⁴ is —(CH₂)ₚ—NR^{a}R^{b}.

In another preferred embodiment, the compound according to formula (I) is a compound according to the following formula

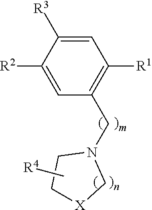

wherein
R¹ is C₂-C₄-alkoxy, wherein each substitutable carbon atom in the aforementioned groups is independently unsubstituted or substituted with one or more, same or different substituents R^{X};
R² is Cl;
R³ is H or Cl; and
R⁴ is —(CH₂)ₚ—NR^{a}R^{b}.

In another preferred embodiment,
X is CH₂, or CHR⁴.

In this connection, it is to be understood that in case X is CHR⁴ the R⁴ substituent on the heterocyclic ring is H.

In another preferred embodiment,
m is 1;
n is 0, 1 or 2, preferably 1 or 2; and
p is 0 or 1, preferably 1.

In another preferred embodiment, the compound of formula (I) is present in the form of a salt, preferably in the form of a hydrochloride salt.

In another preferred embodiment, the compound of formula (I) is selected from the group consisting of (S)-1-(2-ethoxy-5-(trifluoromethyl)benzyl)-3-methylpiperazine hydrochloride, (S)-(1-(2-ethoxy-5-(trifluoromethyl) benzyl) pyrrolidin-3-yl) methanamine hydrochloride, (S)-(1-(5-chloro-2-propoxybenzyl) pyrrolidin-3-yl) methanamine Hydrochloride, (S)-(1-(4,5-dichloro-2-ethoxybenzyl)pyrrolidin-3-yl)methanamine hydrochloride, (S)-(1-(4,5-dichloro-2-(cyclopropylmethoxy)benzyl)pyrrolidin-3-yl)methanamine hydrochloride, (S)-(1-(5-chloro-2-ethoxybenzyl)piperidin-3-yl)methanamine hydrochloride, (S)-(1-(5-chloro-2-(cyclopropylmethoxy)benzyl)piperidin-3-yl)methanamine hydrochloride, (S)-1-(5-chloro-2-ethoxybenzyl)-3-methylpiperazine hydrochloride, 1-(5-chloro-2-ethoxybenzyl)-4-ethylpiperazine, 1-(5-chloro-2-ethoxybenzyl)-N,N-dimethylpiperidin-4-amine, (S)-(1-(5-chloro-2-ethoxybenzyl)pyrrolidin-3-yl)methanamine hydrochloride, (S)-(1-(5-chloro-2-ethoxybenzyl)pyrrolidin-2-yl)methanamine Hydrochloride, (R)-(1-(5-chloro-2-ethoxybenzyl)pyrrolidin-2-yl)methanamine hydrochloride, (R)-1-(5-chloro-2-ethoxybenzyl)piperidin-3-amine hydrochloride, (S)-1-(5-chloro-2-ethoxybenzyl)piperidin-3-amine hydrochloride, (1-(5-chloro-2-ethoxybenzyl)azetidin-3-yl)methanamine Hydrochloride, and 1-(5-chloro-2-ethoxybenzyl)-4-isopropylpiperazine.

In another preferred embodiment, the compound of formula (I) is selected from the group consisting of
(S)-(1-(3,5-dichloro-4-ethoxybenzyl)pyrrolidin-3-yl)methanamine hydrochloride,
1-[2-(3-chloro-4-ethoxyphenyl)ethyl]-4-piperidylamine hydrochloride,
(S)-1-{2-[3,5-dichloro-4-(2-methoxyethoxy)phenyl]ethyl}-3-piperidylamine hydrochloride,
(R)-1-{[3,5-dichloro-4-(cyclopropylmethoxy)phenyl]methyl}-3-pyrrolidinylamine hydrochloride,
(S)-1-{2-[3,5-dichloro-4-(cyclopropylmethoxy)phenyl]ethyl}-3-piperidylamine hydrochloride,
1-[(3-chloro-4-ethoxyphenyl)methyl]-4-piperidylamine hydrochloride,
(R)-1-{2-[3,5-dichloro-4-(2-methoxyethoxy)phenyl]ethyl}-3-piperidylamine hydrochloride,
(S)-1-{[3,5-dichloro-4-(cyclopropylmethoxy)phenyl]methyl}-3-pyrrolidinylamine hydrochloride,
(R)-1-{2-[3,5-dichloro-4-(cyclopropylmethoxy)phenyl]ethyl}-3-piperidylamine hydrochloride,
(S)-1-{2-[3-chloro-4-(cyclopropylmethoxy)phenyl]ethyl}-3-piperidylamine formate,
(S)-1-{2-[3-chloro-4-(cyclopropylmethoxy)phenyl]ethyl}-3-piperidylamine hydrochloride,
(R)-1-{2-[3-chloro-4-(cyclopropylmethoxy)phenyl]ethyl}-3-piperidylamine formate,
(R)-1-{2-[3-chloro-4-(cyclopropylmethoxy)phenyl]ethyl}-3-piperidylamine hydrochloride,
1-{[3-chloro-4-(cyclopropylmethoxy)phenyl]methyl}-4-piperidylamine hydrochloride,
5-{[(R)-3-(Aminomethyl)-1-pyrrolidinyl]methyl}-3-chloro-2-ethoxybenzonitrile hydrochloride,
3-{2-[(S)-3-Amino-1-piperidyl]ethyl}-4-ethoxybenzonitrile hydrochloride,
3-{2-[(R)-3-Amino-1-piperidyl]ethyl}-4-ethoxybenzonitrile hydrochloride,
5-{2-[(S)-3-amino-1-pyrrolidinyl]ethyl}-2-ethoxybenzonitrile hydrochloride,
3-{2-[(S)-3-Amino-1-pyrrolidinyl]ethyl}-4-ethoxybenzonitrile hydrochloride,
3-{2-[(R)-3-amino-1-pyrrolidinyl]ethyl}-4-ethoxybenzonitrile hydrochloride,
(R)-1-[2-(4-ethoxy-3-fluorophenyl)ethyl]-3-pyrrolidinylamine hydrochloride,
(R)-1-{2-[3-chloro-4-(2-methoxyethoxy)phenyl]ethyl}-3-piperidylamine hydrochloride,
1-[(3,5-Dichloro-4-ethoxyphenyl)methyl]-4-piperidylamine hydrochloride,
1-{2-[3,5-Dichloro-4-(cyclopropylmethoxy)phenyl]ethyl}-4-piperidylamine hydrochloride,
(S)-1-[(3-chloro-4-ethoxyphenyl)methyl]-3-piperidylamine hydrochloride,
(R)-1-[(3-chloro-4-ethoxyphenyl)methyl]-3-piperidylamine hydrochloride,
1-{[3,5-dichloro-4-(2-methoxyethoxy)phenyl]methyl}-4-piperidylamine hydrochloride,
1-{[3-chloro-4-(2-methoxyethoxy)phenyl]methyl}-4-piperidylamine hydrochloride,
(S)-1-{[3,5-dichloro-4-(cyclopropylmethoxy)phenyl]methyl}-3-piperidylamine hydrochloride,
(R)-1-{[3,5-dichloro-4-(cyclopropylmethoxy)phenyl]methyl}-3-piperidylamine hydrochloride,
1-{[3,5-dichloro-4-(cyclopropylmethoxy)phenyl]methyl}-4-piperidylamine hydrochloride,
(R)-1-{2-[3-chloro-4-(cyclopropylmethoxy)phenyl]ethyl}-3-pyrrolidinylamine hydrochloride,
(S)-1-{2-[3-chloro-4-(cyclopropylmethoxy)phenyl]ethyl}-3-pyrrolidinylamine hydrochloride,
({(S)-1-[(3-chloro-5-ethoxyphenyl)methyl]-3-pyrrolidinyl}methyl)amine hydrochloride,
and ({1-[(5-chloro-2-ethoxyphenyl)methyl]-3-azetidinyl}methyl)amine hydrochloride.

In a further aspect, the present invention relates to a pharmaceutical composition comprising a pharmaceutically effective amount of the compound of formula (I) as defined herein and optionally a pharmaceutically acceptable carrier, diluent or excipient.

In yet another aspect, the present invention relates to a compound of formula (I) as defined herein or a pharmaceutical composition comprising the same as defined herein for use in medicine. In particular, the present invention relates to a compound of formula (I) as defined herein or a pharmaceutical composition comprising the same as defined herein for inducing and/or stimulating autophagy.

In yet another aspect, the present invention relates to a compound of formula (I) as defined herein or a pharmaceutical composition comprising the same as defined herein for use in a method of treating an autophagy-related disease or condition.

In one embodiment, the compound of the present invention or a pharmaceutical composition comprising the same is for use in the treatment of an autophagy-related disease or condition selected from the group consisting of neurodegenerative diseases, Huntington's disease, Alzheimer's disease, Parkinson's disease, systemic lupus erythematosus, epilepsy, cancer, liver diseases (e.g. nonalcoholic fatty liver disease (NAFLD)), al antitrypsin deficiency, Charcot Marie Tooth syndrome, Rett Syndrome, Sickle Cell disease, Wilson Disease, amyloidosis, Gaucher's diseases, lysosomal and glycogen storage disorders (e.g., Glycogen Storage Disease type 1A (GSD1A)), cystic fibrosis; viral infection and diseases human cytomegalovirus (HCMV) infection, hepatitis B, human immunodeficiency virus infection, Zika virus infection, coronavirus infection, HCoV-229E, HCoV-NL63, betacoronavirus infection, such as HCoV-OC43, SARS-CoV-1, HCoV-HKU1, MERS-CoV or SARS-CoV-2, bacterial infections, metabolic disorders, diabetes, fibrosis, silicosis, diabetic retinopathy, glaucoma, cataracts, age-related macular degeneration, glomerulonephritis, glomerulosclerosis, wound healing disorders, Niemann-Pick type C (NPC) disease, fibrinogen storage disease (FSB), inclusion body disease (IBD), muscular dystrophy, Duchenne muscular dystrophy, Limb-girdle muscular dystrophy, myopathy, myofibrillar myopathy, hereditary myopathy, diabetic cardiomyopathy, anti-inflammatory disorders, autoimmune diseases, multiple sclerosis, rheumatoid arthritis, irritable bowel syndrome, Crohn's disease, vascular disorders, stroke, coronary artery diseases, myocardial infarction, unstable angina pectoris, atherosclerosis or vasculitis, Behcet's syndrome, giant cell arteritis, polymyalgia rheumatic, Wegener's granulomatosis, Churg-Strauss syndrome, vasculitis, Henoch-Schonlein pruprua, Kawasaki disease, viral infection or replication, pox virus infection, herpes virus infection, asthma, allergic rhinitis, COPD, osteoporosis, organ transplant rejection, psoriasis, hypertrophic scarring (keloid formation), adhesion formations following general or gynecological surgery, lung fibrosis, liver fibrosis, kidney fibrosis, disorders caused by intracellular parasites, malaria, tuberculosis, neuropathic pain, post-operative phantom limb pain or postherpetic neuralgia, allergies, amyotrophic lateral sclerosis (ALS), antigen induced recall response, immune response suppression, muscle degeneration and atrophy, frailty in aging, spinal cord injury, and diseases and conditions involving misfolded and/or nonfolded proteins.

In another embodiment of the present invention, said treatment comprises a combination of at least one compound of formula (I) as defined herein with at least one additional pharmaceutically active substance for said autophagy-related disease or condition.

In another aspect, the present invention relates to the use of a compound of formula (I) as defined herein for stimulating autophagy in an in-vitro assay.

### DETAILED DESCRIPTION

In the following, preferred embodiments of the substituents in the above compound of formula (I) are described in further detail. It is to be understood that each preferred embodiment is relevant on its own as well as in combination with other preferred embodiments. Furthermore, it is to be understood that the preferences in each case also apply to the salts, stereoisomers, tautomers and N-oxides of the compounds of formula (I) of the invention. The invention also includes pharmaceutically acceptable salts, solvates, stereoisomers, tautomers and N-oxides of the compound of formula (I).

As indicated above, in a first aspect the present invention relates to a compound of formula (I)

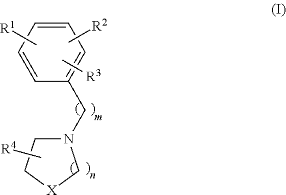

or a salt, stereoisomer, tautomer or N-oxide thereof,
wherein
X is CH₂, CHR⁴, NR^{N} or O;
R¹ is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X};
R² is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X};
R³ is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X}; or
R¹ and R² together with the atoms to which they are attached form a 5- or 6-membered partially unsaturated heterocyclyl, wherein the aforementioned heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{X};
R⁴ is H, C₁-C₄-alkyl, or —(CH₂)ₚ—NR^{a}R^{b};
wherein
R^{N} is H, or C₁-C₄-alkyl;
R^{X} is C₁-C₄-alkyl, C₁-C₄-haloalkyl, NR^{a}R^{b}, or two R^{X} form cyclopropyl;
R^{a}, R^{b} are independently of each other selected from H, and C₁-C₄-alkyl;
R^{c} is H, or C₁-C₄-alkyl;
m is 1, 2, or 3;
n is 0, 1, or 2;
p is 0, 1, 2, or 3.

In one embodiment of the present invention, the following substituent definitions with regard to the substituent R¹ are preferred for the compound of formula (I). Thus, in one embodiment, the present invention relates to a compound according to formula (I)

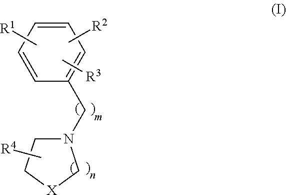

wherein
R¹ is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X},
wherein
R^{X} is C₁-C₄-alkyl, C₁-C₄-haloalkyl, NR^{a}R^{b}, or two R^{X} form cyclopropyl.

In a preferred embodiment of the present invention,
R¹ is C₁-C₄-alkoxy, wherein each substitutable carbon atom in the aforementioned groups is independently unsubstituted or substituted with one or more, same or different substituents R^{X},
wherein
R^{X} is C₁-C₄-alkyl, C₁-C₄-haloalkyl, NR^{a}R^{b}, or two R^{X} form cyclopropyl.

In a more preferred embodiment of the present invention,
R¹ is C₂-C₄-alkoxy, wherein each substitutable carbon atom in the aforementioned groups is independently unsubstituted or substituted with one or more, same or different substituents R^{X},
wherein
R^{X} is C₁-C₄-alkyl, C₁-C₄-haloalkyl, NR^{a}R^{b}, or two R^{X} form cyclopropyl, and wherein preferably two R^{X} form cyclopropyl.

Thus, in a particularly preferred embodiment of the present invention, R¹ is a substituent selected from the group consisting of R¹-1, R¹-2, R¹-3 and R¹-4

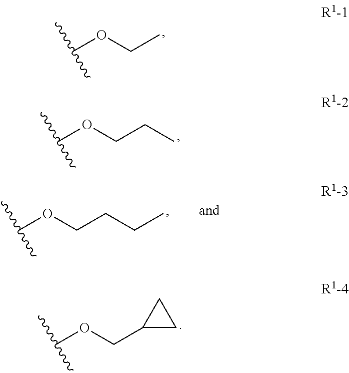

In a particularly preferred embodiment of the present invention, R¹ is selected from R¹-1, R¹-2 and R¹-4.

In connection with the above embodiments, it is to be understood that R², R³, R⁴, X, n and m have the meanings as defined above or further below. Furthermore, in connection with the above embodiments it is to be understood that the compounds according to formula (I) may be present in the form of a salt, preferably the compounds of formula (I) may be present in the form of the hydrochloride salt.

Further, in connection with the above definitions for R¹ it is to be understood that the curled line in the structural formula indicates the connection to the remainder of the molecule.

In another embodiment of the present invention, the following substituent definitions with regard to the substituent R² are preferred for the compound of formula (I). Thus, in one embodiment, the present invention relates to a compound according to formula (I)

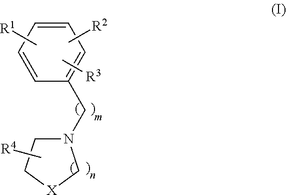

wherein
R² is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X},
wherein
R^{X} is C₁-C₄-alkyl, C₁-C₄-haloalkyl, NR^{a}R^{b}, or two R^{X} form cyclopropyl.

In a preferred embodiment of the present invention,
R² is halogen.

In a more preferred embodiment of the present invention,
R² is F, Cl or Br.

In an even more preferred embodiment of the present invention, R² is Cl.

In connection with the above embodiments, it is to be understood that R¹, R³, R⁴, X, n and m have the meanings as defined above or further below. Furthermore, in connection with the above embodiments it is to be understood that the compounds according to formula (I) may be present in the form of a salt, preferably the compounds of formula (I) may be present in the form of the hydrochloride salt.

In another embodiment of the present invention, R¹ and R² have the following meaning with regard to the compounds of formula (I).

In one embodiment of the present invention,
R¹ and R² together with the atoms to which they are attached form a 5- or 6-membered partially unsaturated heterocyclyl, wherein the aforementioned heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{X},
wherein
R^{X} is C₁-C₄-alkyl, C₁-C₄-haloalkyl, NR^{a}R^{b}, or two R^{X} form cyclopropyl.

In a preferred embodiment of the present invention,
R¹ and R² together with the atoms to which they are attached form a 5- or 6-membered partially unsaturated heterocyclyl, wherein the aforementioned heterocyclic ring comprises two heteroatoms selected from O.

In connection with the above embodiments, it is to be understood that R¹ and R² together with the atoms to which they are attached form a 5- or 6-membered partially unsaturated heterocyclic ring, which is fused to the phenyl ring to which R¹ and R² are attached. Furthermore, it is to be understood that if the fused 5- or 6-membered partially unsaturated heterocyclic ring is further substituted with one or more substituents R^{X}, the one or more substituents R^{X} are selected such, that each C-atom in the 5- or 6-membered partially unsaturated heterocyclic ring does not exceed its valence of four.

The remaining substituents R³, R⁴, X, n and m have the meanings as defined above or further below. Furthermore, in connection with the above embodiments it is to be understood that the compounds according to formula (I) may be present in the form of a salt, preferably the compounds of formula (I) may be present in the form of the hydrochloride salt.

In another embodiment of the present invention, the following substituent definitions with regard to the substituent R³ are preferred for the compound of formula (I). Thus, in one embodiment, the present invention relates to a compound according to formula (I)

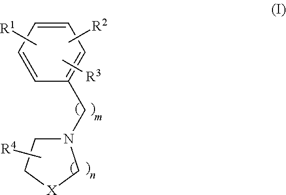

wherein
R³ is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X}, wherein
R^{X} is C₁-C₄-alkyl, C₁-C₄-haloalkyl, NR^{a}R^{b}, or two R^{X} form cyclopropyl.

In a preferred embodiment of the present invention,
R³ is H or halogen.

In another preferred embodiment of the present invention,
R³ is H, F, or C₁.

In a more preferred embodiment of the present invention,
R³ is H or C₁.

In a particularly preferred embodiment of the present invention,
R³ is H.

In connection with the above embodiments, it is to be understood that R¹, R², R⁴, X, n and m have the meanings as defined above or further below. Furthermore, in connection with the above embodiments it is to be understood that the compounds according to formula (I) may be present in the form of a salt, preferably the compounds of formula (I) may be present in the form of the hydrochloride salt.

In another embodiment of the present invention, the following substituent definitions with regard to the substituent R⁴ are preferred for the compounds of formula (I). Thus, in one embodiment, the present invention relates to a compound according to formula (I)

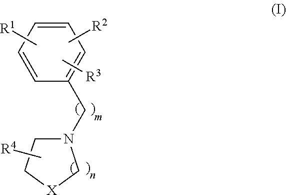

wherein
R⁴ is H, C₁-C₄-alkyl, or —(CH₂)ₚ—NR^{a}R^{b},
wherein
R^{a}, R^{b} are independently of each other selected from H, and C₁-C₄-alkyl, and
p is 0, 1, 2, or 3.

In a preferred embodiment of the present invention,
R⁴ is —(CH₂)ₚ—NR^{a}R^{b},
wherein
R^{a}, R^{b} are independently of each selected from H and CH₃, and
p is 0 or 1.

In a more preferred embodiment of the present invention,
R⁴ is —(CH₂)ₚ—NR^{a}R^{b},
wherein
R^{a}, R^{b} are H, and
p is 0, or 1.

Thus, in a more preferred embodiment of the present invention, R⁴ is selected from the group of R⁴-1, and R⁴-2 having the following structural formula

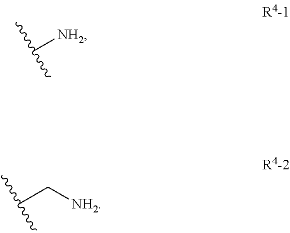

In another more preferred embodiment of the present invention,
R⁴ is —(CH₂)ₚ—NR^{a}R^{b},
wherein
R^{a}, R^{b} are H, and
p is 1 referring to R⁴-2.

In connection with the above embodiments, it is to be understood that R¹, R², R³, X, n and m have the meanings as defined above or further below. Furthermore, in connection with the above embodiments it is to be understood that the compounds according to formula (I) may be present in the form of a salt, preferably the compounds of formula (I) may be present in the form of the hydrochloride salt. Further, in connection with the above definitions for R⁴ it is to be understood that the curled line in the structural formula indicates the connection to the remainder of the molecule.

Thus, in a preferred embodiment the present invention relates to a compound according to formula (I)

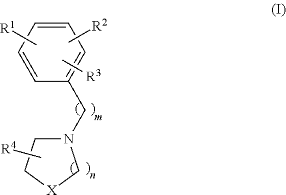

wherein
R¹ is C₁-C₄-alkoxy, wherein each substitutable carbon atom in the aforementioned groups is independently unsubstituted or substituted with one or more, same or different substituents R^{X};
R² is halogen;
R³ is H or halogen; and
R⁴ is —(CH₂)ₚ—NR^{a}R^{b}.

In a more preferred embodiment, the present invention relates to a compound of formula (I)

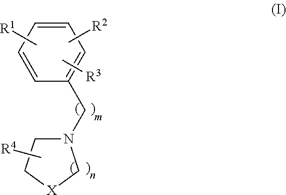

wherein
R¹ is C₂-C₄-alkoxy, wherein each substitutable carbon atom in the aforementioned groups is independently unsubstituted or substituted with one or more, same or different substituents R^{X};
R² is Cl;
R³ is H or Cl; and
R⁴ is —(CH₂)ₚ—NR^{a}R^{b}.

In connection with the above preferred embodiments, it is to be understood that R^{a}, R^{b}, R^{X}, X, m, n and p are as defined above.

In a particularly preferred embodiment of the present invention, the compound according to formula (I) refers to a compound according to the following formula (I)-A

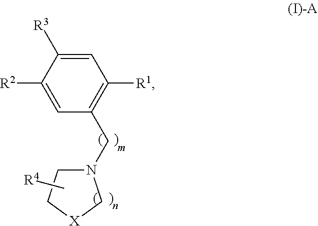

wherein
R¹ is C₂-C₄-alkoxy, wherein each substitutable carbon atom in the aforementioned groups is independently unsubstituted or substituted with one or more, same or different substituents R^{X};
R² is Cl;
R³ is H or Cl; and
R⁴ is —(CH₂)ₚ—NR^{a}R^{b}.

In connection with the compound according to formula (I) and preferably in connection with the compound according to formula (I)-A X, n, and m have the following preferred meanings:
X is CH₂, or CHR⁴;
m is 1; and
n is 0, 1 or 2.

In connection with regard to the substituent X it is to be understood that if X is CH₂ it may be also referred to as X being CHR⁴, depending on where the substituent R⁴ is placed on the heterocyclic ring. Therefore, the meanings for X being CH₂ or CHR⁴ are used interchangeably. In this regard it is to be understood that in case X is CHR⁴ the R⁴ substituent on the heterocyclic ring is H. In other words, if the R⁴ substituent is present at position X in the heterocyclic ring, meaning X is CHR⁴ no further substituent R⁴ is present in the heterocyclic ring, i.e. only one R⁴ is present in the heterocyclic ring. This is further illustrated by the below structural formulae.

Thus, the compound according to formula (I), preferably the compound according to formula (I)-A is preferably a compound according to the following formula (I)-A1, (I)-A2, or (I)-A3

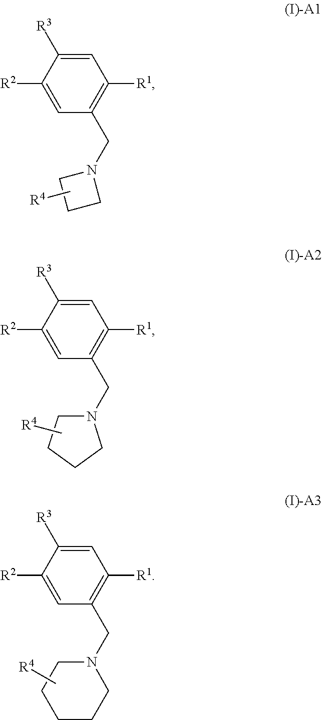

With regard to the compounds according to formula (I), in particular with regard to the compounds according to formula (I)-A, and more particularly with regard to the compounds according to formula (I)-A1, (I)-A2, and (I)-A3 the following substituent meanings are preferred:
R¹ is C₂-C₄-alkoxy, wherein each substitutable carbon atom in the aforementioned groups is independently unsubstituted or substituted with one or more, same or different substituents R^{X};
R² is Cl;
R³ is H or Cl; and
R⁴ is —(CH₂)ₚ—NR^{a}R^{b};
preferably
R¹ is a substituent selected from the group consisting of R¹-1, R¹-2, R¹-3 and R¹-4

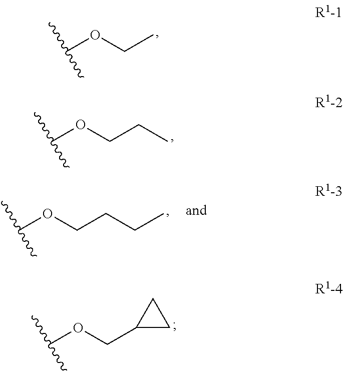

R² is Cl;
R³ is H or C₁, more preferably H; and
R⁴ is

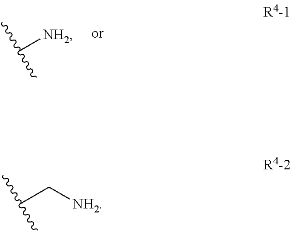

In connection with the above structural formula it is to be understood that the curled line indicates the connection to the remainder of the molecule.

In a preferred embodiment, the compound according to formula (I), preferably the compound according to formula (I)-A, more preferably the compound according to formula (I)-A1, (I)-A2, and (I)-A3 is a compound according to the following formula (I)-A1*, (I)-A2*, (I)-A2**, or (I)-A3*

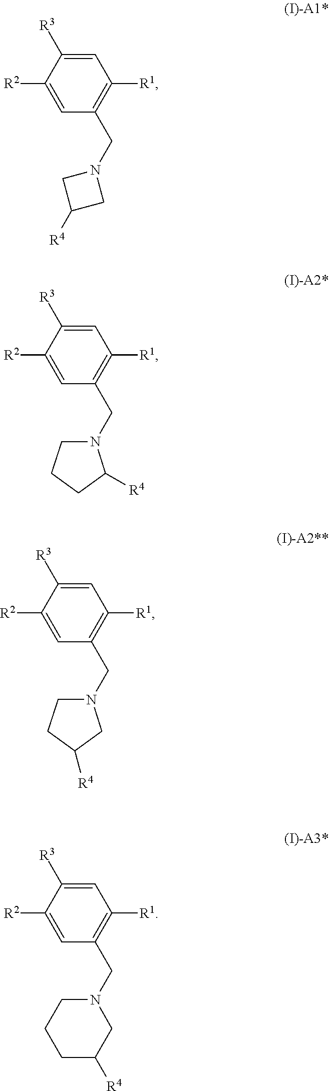

With regard to the compounds according to formula (I), in particular with regard to the compounds according to formula (I)-A, more particularly with regard to the compounds according to formula (I)-A1, (I)-A2, and (I)-A3, and even more particularly with regard to the compounds according to formula (I)-A1*, (I)-A2*, (I)-A2**, or (I)-A3* the following substituent meanings are preferred:
R¹ is a substituent selected from the group consisting of R¹-1, R¹-2, R¹-3 and R¹-4

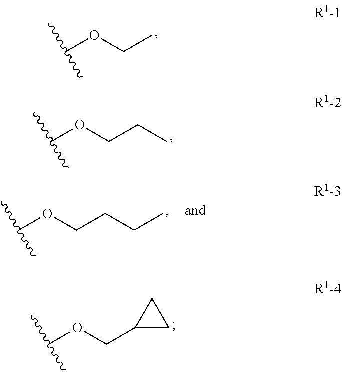

R² is Cl;
R³ is H or C₁, more preferably H;
R⁴ is

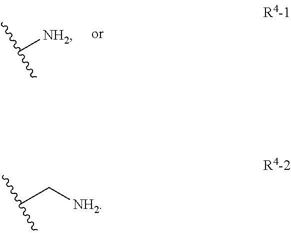

In a more preferred embodiment of the present invention, the compound according to formula (I) is a compound according to formula (I)-A2**
wherein
R¹ is

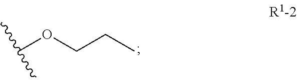 [SMILES: *OCCC]

R² is Cl;
R³ is H; and
R⁴ is

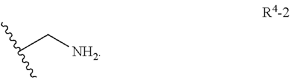 [SMILES: *CN]

In connection with the above preferred embodiment, it is to be understood that the compound is preferably present in the form of a hydrochloride salt.

In another more preferred embodiment of the present invention, the compound according to formula (I) is a compound according to formula (I)-A2**
wherein
R¹ is

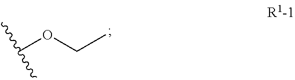 [SMILES: *OCC]

R² is Cl;
R³ is Cl; and
R⁴ is

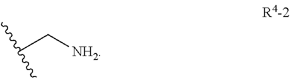 [SMILES: *CN]

In connection with the above preferred embodiment, it is to be understood that the compound is preferably present in the form of a hydrochloride salt.

In another more preferred embodiment of the present invention, the compound according to formula (I) is a compound according to formula (I)-A2**
wherein
R¹ is

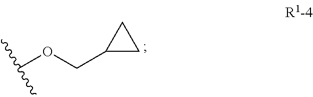 [SMILES: *OCC1CC1]

R² is Cl;
R³ is Cl; and
R⁴ is

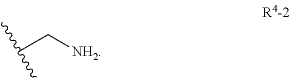 [SMILES: *CN]

In connection with the above preferred embodiment, it is to be understood that the compound is preferably present in the form of a hydrochloride salt.

In another more preferred embodiment of the present invention, the compound according to formula (I) is a compound according to formula (I)-A3*
wherein
R¹ is

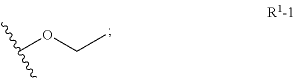 [SMILES: *OCC]

R² is Cl;
R³ is H; and
R⁴ is

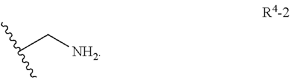 [SMILES: *CN]

In connection with the above preferred embodiment, it is to be understood that the compound is preferably present in the form of a hydrochloride salt.

In another more preferred embodiment of the present invention, the compound according to formula (I) is a compound according to formula (I)-A3*
wherein
R¹ is

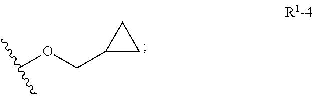 [SMILES: *OCC1CC1]

R² is Cl;
R³ is H; and
R⁴ is

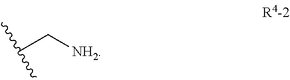 [SMILES: *CN]

In connection with the above preferred embodiment, it is to be understood that the compound is preferably present in the form of a hydrochloride salt.

In another more preferred embodiment of the present invention, the compound according to formula (I) is a compound according to formula (I)-A2*
wherein
R¹ is

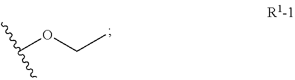 [SMILES: *OCC]

R² is Cl;
R³ is H; and
R⁴ is

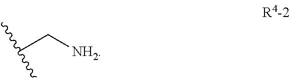 [SMILES: *CN]

In connection with the above preferred embodiment, it is to be understood that the compound is preferably present in the form of a hydrochloride salt.

In another more preferred embodiment of the present invention, the compound according to formula (I) is a compound according to formula (I)-A3*
wherein
R¹ is

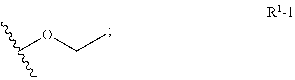 [SMILES: *OCC]

R² is Cl;
R³ is H; and
R⁴ is

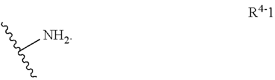 [SMILES: *N]

In connection with the above preferred embodiment, it is to be understood that the compound is preferably present in the form of a hydrochloride salt.

In a preferred embodiment of the present invention, the compound according to formula (I) is a compound according to formula (I)-A1*
wherein
R¹ is

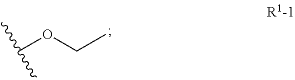 [SMILES: *OCC]

R² is Cl;
R³ is H; and
R⁴ is

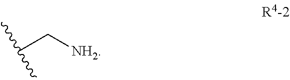 [SMILES: *CN]

In connection with the above preferred embodiment, it is to be understood that the compound is preferably present in the form of a hydrochloride salt.

In connection with the above compounds according to formula (I), preferably according to formula (I)-A, more preferably according to formula (I)-A1, (I)-A2, or (I)-A3, particularly preferably according to formula (I)-A1*, (I)-A2*, (I)-A2**, or (I)-A3* it is to be understood that the compounds according to formula (I) are preferably present in the form of a salt, more preferably in the form of the hydrochloride salt. Further, in connection with the above structural formula it is to be understood that the curled line indicates the connection to the remainder of the molecule.

In connection with the above compounds according to formula (I), and according to formula (I)-A, it is to be understood that the compound of formula (I) may be also resembled by a compound according to formula (I)-B with the following structural formula

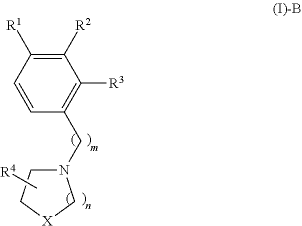

wherein
R¹ is C₂-C₄-alkoxy, wherein each substitutable carbon atom in the aforementioned groups is independently unsubstituted or substituted with one or more, same or different substituents R^{X};
R² is Cl;
R³ is H or Cl; and
R⁴ is —(CH₂)ₚ—NR^{a}R^{b}.

In connection with the compound according to formula (I) and preferably in connection with the compound according to formula (I)-B X, n, and m have the following preferred meanings:
X is CH₂, or CHR⁴;
m is 1; and
n is 0, 1 or 2, preferably n is 1.

In connection with regard to the substituent X it is to be understood that if X is CH₂ it may be also referred to as X being CHR⁴, depending on where the substituent R⁴ is placed on the heterocyclic ring. Therefore, the meanings for X being CH₂ or CHR⁴ are used interchangeably. In this regard it is to be understood that in case X is CHR⁴ the R⁴ substituent on the heterocyclic ring is H. In other words, if the R⁴ substituent is present at position X in the heterocyclic ring, meaning X is CHR⁴ no further substituent R⁴ is present in the heterocyclic ring, i.e. only one R⁴ is present in the heterocyclic ring. This is further illustrated by the below structural formula.

In a preferred embodiment, the compound according to formula (I), preferably the compound according to formula (I)-B, is a compound according to the following formula (I)-B1

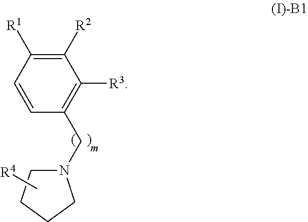

With regard to the compounds according to formula (I), in particular with regard to the compounds according to formula (I)-B, more particularly with regard to the compounds according to formula (I)-B1, the following substituent meanings are preferred: R¹ is a substituent selected from the group consisting of R¹-1, R¹-2, R¹-3 and R¹-4

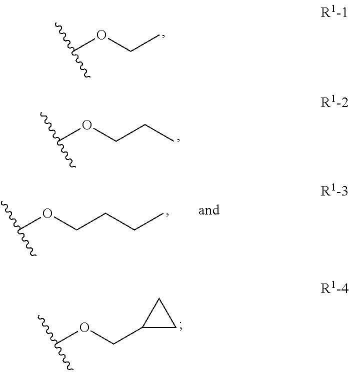

more preferably

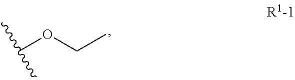 [SMILES: *OCC]

R² is;
R³ is H or Cl, more preferably H;
R⁴ is

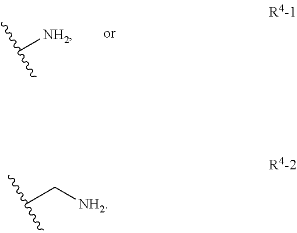

In connection with the above structural formula it is to be understood that the curled line indicates the connection to the remainder of the molecule.

In a more preferred embodiment, the compound according to formula (I), preferably the compound according to formula (I)-B, more preferably the compound of formula (I)-B1, is a compound according to the following formula (I)-B1*

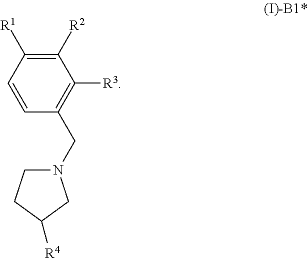

With regard to the compounds according to formula (I), in particular with regard to the compounds according to formula (I)-B, more particularly with regard to the compounds according to formula (I)-B1* the following substituent meanings are preferred:
R¹ is a substituent selected from the group consisting of R¹-1, R¹-2, R¹-3 and R¹-4

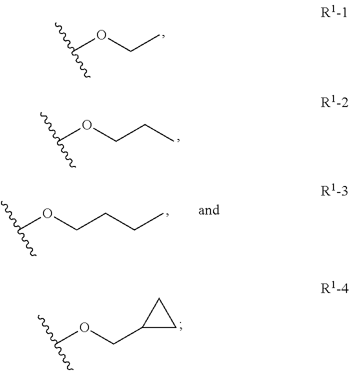

more preferably

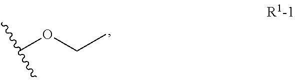 [SMILES: *OCC]

R² is Cl;
R³ is H or Cl, more preferably H;
R⁴ is

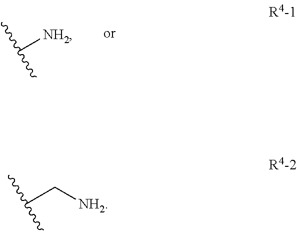

In a preferred embodiment of the present invention, the compound according to formula (I) is a compound according to formula (I)-B1*
wherein
R¹ is

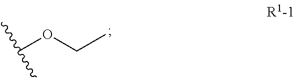 [SMILES: *OCC]

R² is Cl;
R³ is H; and
R⁴ is

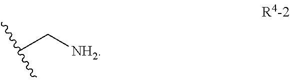 [SMILES: *CN]

In connection with the above compounds according to formula (I), preferably according to formula (I)-B, more preferably according to formula (I)-B1, even more preferably according to formula (I)-B1* it is to be understood that the compounds according to formula (I) are preferably present in the form of a salt, more preferably in the form of the hydrochloride salt. Further, in connection with the above structural formula it is to be understood that the curled line indicates the connection to the remainder of the molecule.

In connection with the above compounds according to formula (I), it is to be understood that the compound of formula (I) may be also resembled by a compound according to formula (I)-C with the following structural formula

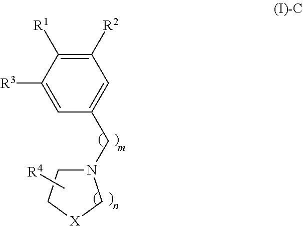

wherein
R¹ is C₂-C₄-alkoxy, wherein each substitutable carbon atom in the aforementioned groups is independently unsubstituted or substituted with one or more, same or different substituents R^{X};
R² is Cl;
R³ is H or Cl; and
R⁴ is —(CH₂)ₚ—NR^{a}R^{b}.

In connection with the compound according to formula (I) and preferably in connection with the compound according to formula (I)-C X, n, and m have the following preferred meanings:
X is CH₂, or CHR⁴;
m is 1 or 2; and
n is 0, 1 or 2, preferably n is 1 or 2.

In connection with regard to the substituent X it is to be understood that if X is CH₂ it may be also referred to as X being CHR⁴, depending on where the substituent R⁴ is placed on the heterocyclic ring. Therefore, the meanings for X being CH₂ or CHR⁴ are used interchangeably. In this regard it is to be understood that in case X is CHR⁴ the R⁴ substituent on the heterocyclic ring is H. In other words, if the R⁴ substituent is present at position X in the heterocyclic ring, meaning X is CHR⁴ no further substituent R⁴ is present in the heterocyclic ring, i.e. only one R⁴ is present in the heterocyclic ring. This is further illustrated by the below structural formula.

Thus, the compound according to formula (I), preferably the compound according to formula (I)-C is preferably a compound according to the following formula (I)-C1 or (I)-C2

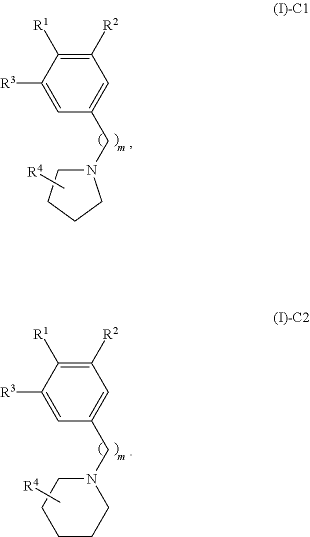

With regard to the compounds according to formula (I), in particular with regard to the compounds according to formula (I)-C, more particularly with regard to the compounds according to formula (I)-C1 and (I)-C2 the following substituent meanings are preferred:
R¹ is a substituent selected from the group consisting of R¹-1, R¹-2, R¹-3 and R¹-4

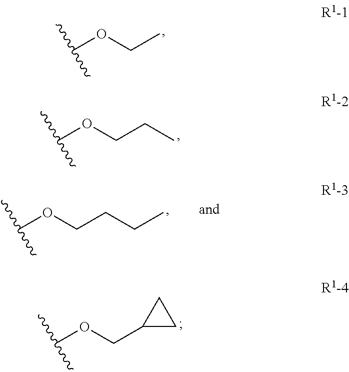

R² is Cl;
R³ is H or Cl;
R⁴ is

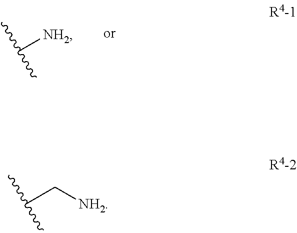

In a preferred embodiment of the present invention, the compound according to formula (I) is a compound according to formula (I)-C1 or (I)-C2
wherein
R¹ is

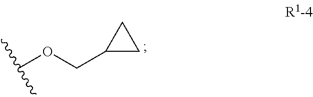 [SMILES: *OCC1CC1]

R² is Cl;
R³ is H or Cl; and
R⁴ is

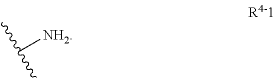 [SMILES: *N]

In a preferred embodiment, the compound according to formula (I), preferably the compound according to formula (I)-C, more preferably the compound according to formula (I)-C1 and (I)-C2, is a compound according to the following formula (I)-C1* or (I)-C2*

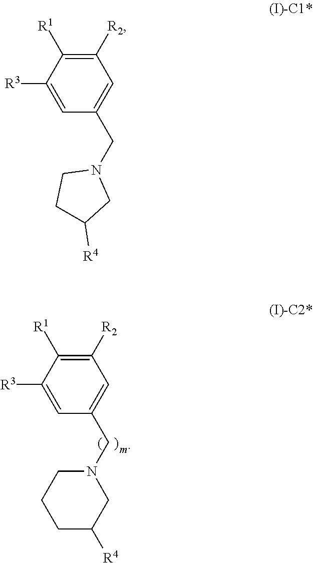

With regard to the compounds according to formula (I), in particular with regard to the compounds according to formula (I)-C, more particularly with regard to the compounds according to formula (I)-C1 and (I)-C2, and even more particularly with regard to the compounds according to formula (I)-C1* and (I)-C2* the following substituent meanings are preferred:
R¹ is a substituent selected from the group consisting of R¹-1, R¹-2, R¹-3 and R¹-4

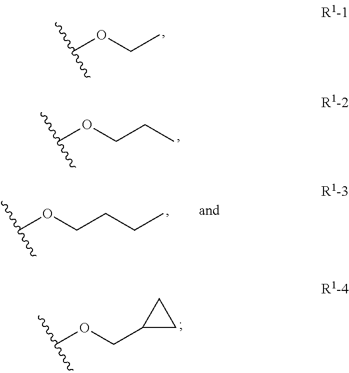

more preferably

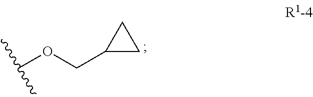 [SMILES: *OCC1CC1]

R² is Cl;
R³ is H or Cl;
R⁴ is

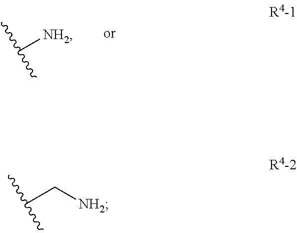

preferably

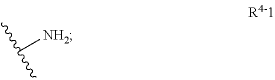 [SMILES: *N]

m is 1 or 2, preferably 2.

In a more preferred embodiment of the present invention, the compound according to formula (I) is a compound according to formula (I)-C1*
wherein
R¹ is

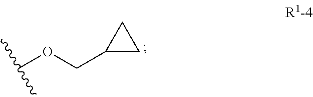 [SMILES: *OCC1CC1]

R² is H or Cl;
R³ is Cl; and
R⁴ is

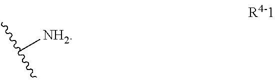 [SMILES: *N]

In another more preferred embodiment of the present invention, the compound according to formula (I) is a compound according to formula (I)-C2*
wherein
R¹ is

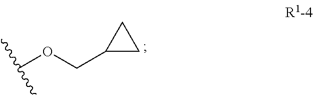 [SMILES: *OCC1CC1]

R² is H or Cl;
R³ is Cl;
R⁴ is

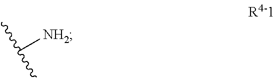 [SMILES: *N]

and
m is 2.

In connection with the above compounds according to formula (I), preferably according to formula (I)-C, more preferably according to formula (I)-C1 and (I)-C2, even more preferably according to formula (I)-C1* and (I)-C2*, it is to be understood that the compounds according to formula (I) are preferably present in the form of a salt, more preferably in the form of the hydrochloride salt. Further, in connection with the above structural formula it is to be understood that the curled line indicates the connection to the remainder of the molecule.

In connection with the compounds of formula (I), as well as in connection with the compounds of formula (I)-A or (I)-B, and in connection with the compounds of formula (I)-A1, (I)-A2, and (I)-A3, especially in connection with the compounds of formula (I)-A1*, (I)-A2*, (I)-A2**, (I)-A3* and (I)-B1* it is preferred that the compound is not

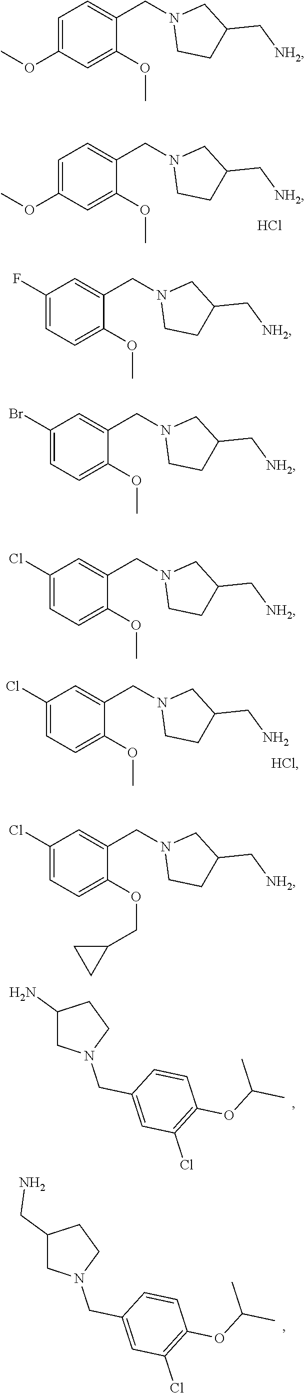

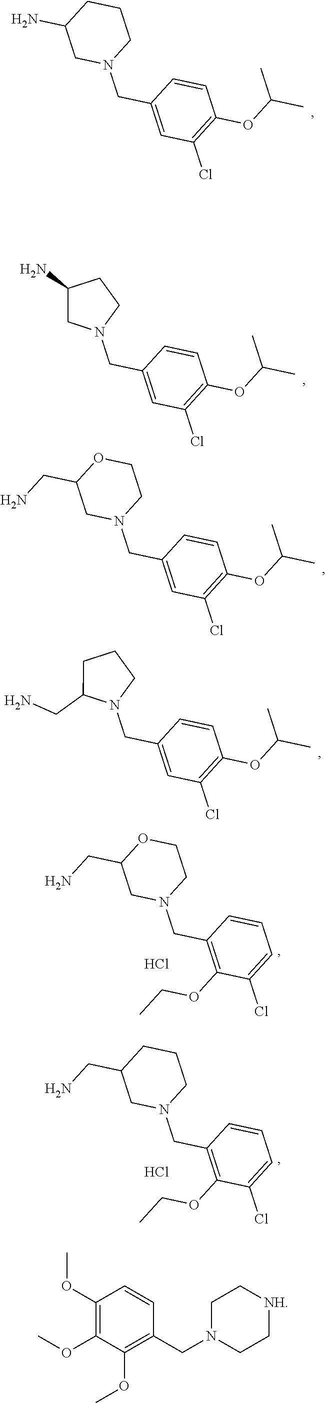

In connection with the compounds of formula (I), as well as in connection with the compounds of formula (I)-A or (I)-B, and in connection with the compounds of formula (I)-A1, (I)-A2, and (I)-A3, especially in connection with the compounds of formula (I)-A1*, (I)-A2*, (I)-A2**, (I)-A3* and (I)-B1* it is preferred that the compound is not

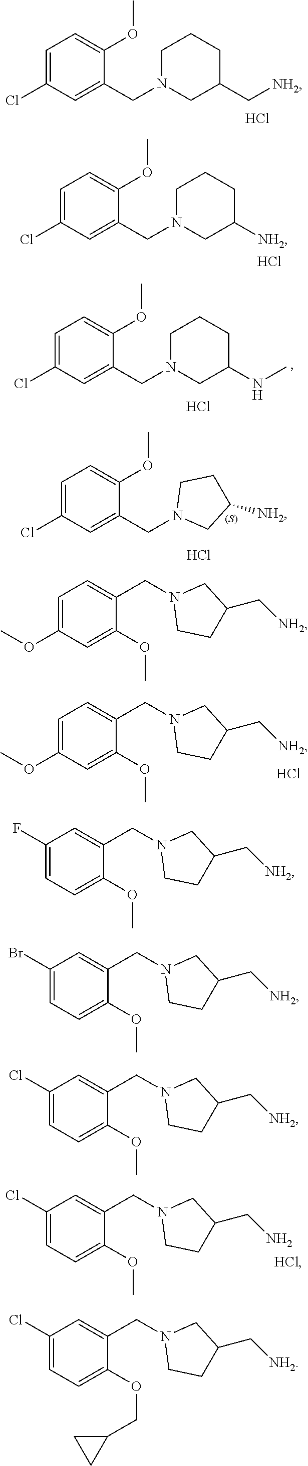

In connection with the compounds of formula (I), as well as in connection with the compounds of formula (I)-A or (I)-B, and in connection with the compounds of formula (I)-A1, (I)-A2, and (I)-A3, especially in connection with the compounds of formula (I)-A1*, (I)-A2*, (I)-A2**, (I)-A3* and (I)-B1* it is preferred that the compound is not

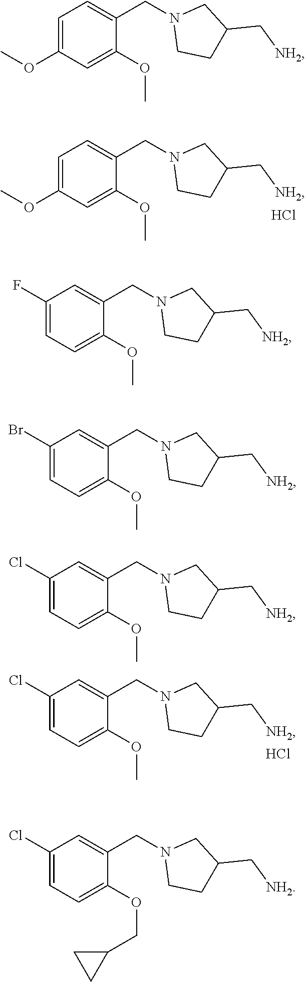

Preferably, in connection with the compounds of formula (I)-A2** it is preferred that the compound is not

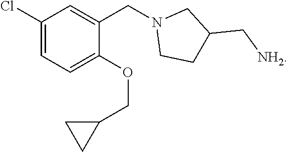 [SMILES: NCC1CCN(Cc2cc(Cl)ccc2OCC2CC2)C1]

In a more preferred embodiment of the present invention, the compound according to formula (I) is a compound according to formula (I)-A or formula (I)-B, preferably a compound according to formula (I)-A2 or formula (I)-B1, particularly preferably a compound according to formula (I)-A2** or formula (I)-B1*;
wherein
R¹ is

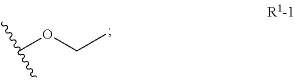 [SMILES: *OCC]

R² is Cl;
R³ is H; and
R⁴ is

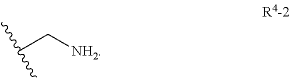 [SMILES: *CN]

In connection with the above preferred embodiment, it is to be understood that the compound is preferably present in the form of a hydrochloride salt.

In connection with the above structural formula it is to be understood that the curled line indicates the connection to the remainder of the molecule.

In a particularly preferred embodiment of the present invention, the compound according to formula (I) is a compound according to formula (I)-A, preferably a compound according to formula (I)-A2, particularly preferably a compound according to formula (I)-A2**,
wherein
R¹ is

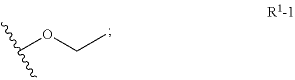 [SMILES: *OCC]

R² is Cl;
R³ is H; and
R⁴ is

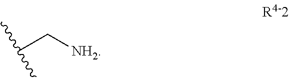 [SMILES: *CN]

In connection with the above preferred embodiment, it is to be understood that the compound is preferably present in the form of a hydrochloride salt.

In connection with the above structural formula it is to be understood that the curled line indicates the connection to the remainder of the molecule.

In another particularly preferred embodiment of the present invention, the compound according to formula (I) is a compound according to formula (I)-B, preferably a compound according to formula (I)-B1, particularly preferably a compound according to formula (I)-B1*,
wherein
R¹ is

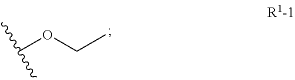 [SMILES: *OCC]

R² is Cl;
R³ is H; and
R⁴ is

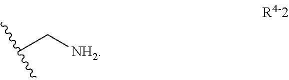 [SMILES: *CN]

In connection with the above preferred embodiment, it is to be understood that the compound is preferably present in the form of a hydrochloride salt.

In connection with the above structural formula it is to be understood that the curled line indicates the connection to the remainder of the molecule.

In another more preferred embodiment of the present invention, the compound according to formula (I) is a compound according to formula (I)-C, preferably a compound according to formula (I)-C1 or formula (I)-C2, particularly preferably a compound according to formula (I)-C1* or formula (I)-C2*;
wherein
R¹ is

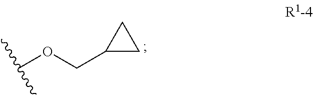 [SMILES: *OCC1CC1]

R² is H or Cl;
R³ is Cl;
R⁴ is

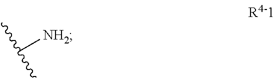 [SMILES: *N]

and
m is 2.

In connection with the above preferred embodiment, it is to be understood that the compound is preferably present in the form of a hydrochloride salt.

In connection with the above structural formula it is to be understood that the curled line indicates the connection to the remainder of the molecule.

It has been found that the compounds as defined above are particularly advantageous for inducing and/or stimulating autophagy, and may therefore particularly advantageously be used in the pharmaceutical compositions of the present invention as well as medical uses as defined herein. Therefore, the compound of formula (I) of the invention is preferably a compound according to formula (I)-A1*, (I)-A2*, (I)-A2** or (I)-A3* as defined above and the present invention preferably relates to pharmaceutical compositions comprising the same and to medical uses thereof.

Further, the compound of formula (I) of the invention is preferably a compound according to formula (I)-B1* as defined above and the present invention preferably relates to pharmaceutical compositions comprising the same and to medical uses thereof.

Further, the compound of formula (I) of the invention is preferably a compound according to formula (I)-C1* or (I)-C2* as defined above and the present invention preferably relates to pharmaceutical compositions comprising the same and to medical uses thereof.

In a second aspect the present invention relates to a compound of formula (I)

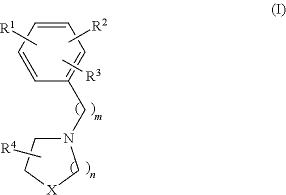

or a salt, stereoisomer, tautomer or N-oxide thereof,
wherein
X is CH₂, CHR⁴, NR^{N} or O;
R¹ is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X};
R² is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X};
R³ is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X}; or
R¹ and R² together with the atoms to which they are attached form a 5- or 6-membered partially unsaturated heterocyclyl, wherein the aforementioned heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{X};
R⁴ is H, C₁-C₄-alkyl, or —(CH₂)ₚ—NR^{a}R^{b};
wherein
R^{N} is H, or C₁-C₄-alkyl;
R^{X} is C₁-C₄-alkyl, C₁-C₄-haloalkyl, NR^{a}R^{b}, or two R^{X} form cyclopropyl;
R^{a}, R^{b} are independently of each other selected from H, and C₁-C₄-alkyl;
R^{c} is H, or C₁-C₄-alkyl;
m is 1, 2, or 3;
n is 0, 1, or 2;
p is 0, 1, 2, or 3.

The following substituent definitions are preferred with regard to the compounds according to formula (I) of the second aspect of the present invention.

In one embodiment of the second aspect of the present invention with regard to the compounds of formula (I)
X is NR^{N} or O,
preferably
X is NR^{N}.

In connection with the above embodiment, it is to be understood that
R^{N} is H, or C₁-C₄-alkyl,
preferably
R^{N} is H, CH₃, CH₂CH₃, or CH(CH₃)₂.

In another embodiment of the second aspect of the present invention with regard to the compounds of formula (I)
R¹ is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X};
preferably
R¹ is C₁-C₄-alkoxy,
more preferably
R¹ is C₂-alkoxy.

In another embodiment of the second aspect of the present invention with regard to the compounds of formula (I)
R² is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X};
preferably
R² is C₁-C₄-haloalkyl, or halogen;
more preferably
R² is CF₃, or C₁.

In another embodiment of the second aspect of the present invention with regard to the compounds of formula (I)
R³ is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X}; and
R⁴ is H, C₁-C₄-alkyl, or —(CH₂)ₚ—NR^{a}R^{b},
preferably
R³ is H; and
R⁴ is H or C₁-C₄-alkyl, preferably H or CH₃.

In another embodiment of the second aspect of the present invention with regard to the compounds of formula (I)
m is 1; and
n is 1 or 2, preferably 2.

In a preferred embodiment of the second aspect of the present invention the compound according to formula (I) is a compound according to formula (I)-A

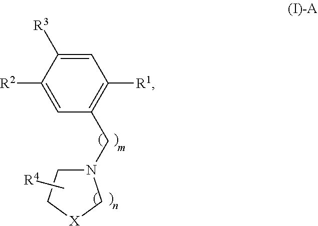

wherein
X is NR^{N}; wherein
R^{N} is H, CH₃, CH₂CH₃, or CH(CH₃)₂;
R¹ is C₂-alkoxy;
R² is CF₃, or Cl;
R³ is H;
R⁴ is H or C₁-C₄-alkyl, preferably H or CH₃;
m is 1; and
n is 2

In particularly preferred embodiments, the compound of formula (I) is a compound selected from the group consisting of (S)-1-(2-ethoxy-5-(trifluoromethyl)benzyl)-3-methylpiperazine hydrochloride, (S)-(1-(2-ethoxy-5-(trifluoromethyl) benzyl) pyrrolidin-3-yl) methanamine hydrochloride, (S)-(1-(5-chloro-2-propoxybenzyl) pyrrolidin-3-yl) methanamine Hydrochloride, (S)-(1-(4,5-dichloro-2-ethoxybenzyl)pyrrolidin-3-yl)methanamine hydrochloride, (S)-(1-(4,5-dichloro-2-(cyclopropylmethoxy)benzyl)pyrrolidin-3-yl)methanamine hydrochloride, (S)-(1-(5-chloro-2-ethoxybenzyl)piperidin-3-yl)methanamine hydrochloride, (S)-(1-(5-chloro-2-(cyclopropylmethoxy)benzyl)piperidin-3-yl)methanamine hydrochloride, (S)-1-(5-chloro-2-ethoxybenzyl)-3-methylpiperazine hydrochloride, 1-(5-chloro-2-ethoxybenzyl)-4-ethylpiperazine, 1-(5-chloro-2-ethoxybenzyl)-N,N-dimethylpiperidin-4-amine, (S)-(1-(5-chloro-2-ethoxybenzyl)pyrrolidin-3-yl)methanamine hydrochloride, (S)-(1-(5-chloro-2-ethoxybenzyl)pyrrolidin-2-yl)methanamine Hydrochloride, (R)-(1-(5-chloro-2-ethoxybenzyl)pyrrolidin-2-yl)methanamine hydrochloride, (R)-1-(5-chloro-2-ethoxybenzyl)piperidin-3-amine hydrochloride, (S)-1-(5-chloro-2-ethoxybenzyl)piperidin-3-amine hydrochloride, (1-(5-chloro-2-ethoxybenzyl)azetidin-3-yl)methanamine Hydrochloride, and 1-(5-chloro-2-ethoxybenzyl)-4-isopropylpiperazine.

In another particularly preferred embodiment, the compound of formula (I) is a compound selected from the group consisting of
(S)-1-(5-chloro-2-ethoxyphenethyl)piperidin-3-amine hydrochloride,
(S)-(1-(3-chloro-4-(cyclopropylmethoxy)phenethyl)pyrrolidin-3-yl)methanamine hydrochloride,
(R)-(1-(2-ethoxy-4,5-difluorobenzyl)pyrrolidin-3-yl)methanamine hydrochloride,
(S)-(1-(2-ethoxy-4,5-difluorobenzyl)pyrrolidin-3-yl)methanamine hydrochloride,
(R)-(1-(3-chloro-4-ethoxybenzyl)pyrrolidin-3-yl) methanamine hydro chloride,
(R)-2-(2-((3-(aminomethyl)pyrrolidin-1-yl)methyl)-4-chlorophenoxy)ethan-1-ol hydrochloride,
(S)-2-(2-((3-(aminomethyl)pyrrolidin-1-yl)methyl)-4-chlorophenoxy)ethan-1-ol hydrochloride,
(R)-(1-(2-butoxy-5-chlorobenzyl)pyrrolidin-3-yl)methanamine hydrochloride,
(S)-(1-(2-butoxy-5-chlorobenzyl)pyrrolidin-3-yl)methanamine hydrochloride,
(S)-(4-(5-chloro-2-ethoxybenzyl)morpholin-2-yl)methanamine hydrochloride,
(R)-(4-(5-chloro-2-ethoxybenzyl)morpholin-2-yl)methanamine hydrochloride,
(S)-(1-(3-chloro-4-ethoxybenzyl)pyrrolidin-3-yl)methanamine hydrochloride,
(S)-(1-(3-chloro-5-ethoxybenzyl)pyrrolidin-3-yl)methanamine hydro chloride,
(S)-3-methyl-1-(2-propoxy-5-(trifluoromethoxy)benzyl) piperazine hydrochloride,
(S)-(1-(2-(6-chlorobenzo[d][1,3]dioxol-4-yl)ethyl)pyrrolidin-3-yl)methanamine hydrochloride,
(S)-(1-(2,3-dichloro-6-ethoxyphenethyl)pyrrolidin-3-yl)methanamine hydrochloride,
(S)-(1-(4,5-dichloro-2-ethoxyphenethyl)pyrrolidin-3-yl)methanamine hydrochloride,
(R)-1-(2-ethoxy-5-(trifluoromethyl)benzyl)-3-methylpiperazine hydrochloride,
(S)-1-(4,5-dichloro-2-ethoxybenzyl)-3-methylpiperazine hydrochloride,
(S)-1-(2-ethoxy-5-(trifluoromethoxy)benzyl)-3-methylpiperazine hydrochloride,
(1-(3-chloro-4-ethoxybenzyl)pyrrolidin-3-yl)methanamine hydrochloride,
1-(5-chloro-2-ethoxybenzyl)piperazine hydrochloride,
(4-(5-chloro-2-ethoxybenzyl)morpholin-2-yl)methanamine hydrochloride,
(S)-(1-(2-ethoxy-5-(trifluoromethyl)phenethyl)pyrrolidin-3-yl)methanamine hydrochloride,
(S)-(1-(5-chloro-2-propoxyphenethyl)piperidin-3-yl)methanamine hydrochloride,
(S)-(1-(5-chloro-2-(cyclopropylmethoxy)phenethyl)pyrrolidin-3-yl)methanamine hydrochloride,
(S)-(1-(5-chloro-2-ethoxyphenethyl)piperidin-3-yl)methanamine hydrochloride,
(S)-1-(5-chloro-2-(cyclopropylmethoxy)benzyl)-3-methylpiperazine hydrochloride,
(S)-1-(5-chloro-2-propoxybenzyl)-3-methylpiperazine hydrochloride,
(S)-(1-(5-chloro-2-propoxyphenethyl)pyrrolidin-3-yl)methanamine hydrochloride,
(S)-(1-(5-chloro-2-ethoxyphenethyl)pyrrolidin-3-yl)methanamine hydrochloride,
(S)-1-(1-(5-chloro-2-ethoxybenzyl)pyrrolidin-3-yl)-N,N-dimethyl methanamine,
(S)-1-(1-(5-chloro-2-ethoxybenzyl)pyrrolidin-3-yl)-N-methylmethanamine,
(S)-(1-(5-chloro-2-isobutoxybenzyl)pyrrolidin-3-yl)methanamine hydrochloride,
(S)-(1-(5-chloro-2-isopropoxybenzyl)pyrrolidin-3-yl)methanamine hydrochloride,
(S)-(1-(5-chloro-2-(2-methoxyethoxy)benzyl)pyrrolidin-3-yl)methanamine hydrochloride,
(S)-(1-((6-chlorobenzo[d][1,3]dioxol-4-yl)methyl)pyrrolidin-3-yl)methanamine hydrochloride,
(R)-(1-(5-chloro-2-ethoxybenzyl)pyrrolidin-3-yl)methanamine hydrochloride,
(S)-(1-(3-chloro-4-ethoxy-2-fluorobenzyl)pyrrolidin-3-yl)methanamine hydrochloride,
(S)-(1-(5-chloro-4-ethoxy-2-fluorobenzyl)pyrrolidin-3-yl)methanamine hydrochloride,
(R)-4-((3-(aminomethyl)pyrrolidin-1-yl)methyl)-2-chloro-5-ethoxy benzonitrile hydrochloride,
(S)-4-((3-(aminomethyl)pyrrolidin-1-yl)methyl)-2-chloro-5-ethoxybenzonitrile hydrochloride,
(S)-(4-(5-chloro-2-ethoxyphenethyl)morpholin-2-yl)methanamine hydrochloride,
(S)-3-(2-(3-(aminomethyl)piperidin-1-yl)ethyl)-4-ethoxybenzonitrile hydrochloride,
(S)-3-(2-(3-(aminomethyl)pyrrolidin-1-yl)ethyl)-4-(cyclopropylmethoxy)benzonitrile hydrochloride,
(S)-(1-(3-chloro-4-ethoxyphenethyl)piperidin-3-yl)methanamine hydrochloride, and
(S)-1-(5-chloro-2-ethoxybenzyl)pyrrolidin-3-amine hydrochloride.

In another particularly preferred embodiment, the compound of formula (I) is a compound selected from the group consisting of (S)-1-(2-ethoxy-5-(trifluoromethyl)benzyl)-3-methylpiperazine hydrochloride, (S)-(1-(2-ethoxy-5-(trifluoromethyl) benzyl) pyrrolidin-3-yl) methanamine hydrochloride, (S)-(1-(5-chloro-2-propoxybenzyl) pyrrolidin-3-yl) methanamine Hydrochloride, (S)-(1-(4,5-dichloro-2-ethoxybenzyl)pyrrolidin-3-yl)methanamine hydrochloride, (S)-(1-(4,5-dichloro-2-(cyclopropylmethoxy)benzyl)pyrrolidin-3-yl)methanamine hydrochloride, (S)-(1-(5-chloro-2-ethoxybenzyl)piperidin-3-yl)methanamine hydrochloride, (S)-(1-(5-chloro-2-(cyclopropylmethoxy)benzyl)piperidin-3-yl)methanamine hydrochloride, (S)-1-(5-chloro-2-ethoxybenzyl)-3-methylpiperazine hydrochloride, 1-(5-chloro-2-ethoxybenzyl)-4-ethylpiperazine, 1-(5-chloro-2-ethoxybenzyl)-N,N-dimethylpiperidin-4-amine, (S)-(1-(5-chloro-2-ethoxybenzyl)pyrrolidin-3-yl)methanamine hydrochloride, (S)-(1-(5-chloro-2-ethoxybenzyl)pyrrolidin-2-yl)methanamine Hydrochloride, (R)-(1-(5-chloro-2-ethoxybenzyl)pyrrolidin-2-yl)methanamine hydrochloride, (R)-1-(5-chloro-2-ethoxybenzyl)piperidin-3-amine hydrochloride, (S)-1-(5-chloro-2-ethoxybenzyl)piperidin-3-amine hydrochloride, (1-(5-chloro-2-ethoxybenzyl)azetidin-3-yl)methanamine Hydrochloride, 1-(5-chloro-2-ethoxybenzyl)-4-isopropylpiperazine, (S)-1-(5-chloro-2-ethoxyphenethyl)piperidin-3-amine hydrochloride, (S)-(1-(3-chloro-4-(cyclopropylmethoxy)phenethyl)pyrrolidin-3-yl)methanamine hydrochloride, (R)-(1-(2-ethoxy-4,5-difluorobenzyl)pyrrolidin-3-yl)methanamine hydrochloride, (S)-(1-(2-ethoxy-4,5-difluorobenzyl)pyrrolidin-3-yl)methanamine hydrochloride, (R)-(1-(3-chloro-4-ethoxybenzyl)pyrrolidin-3-yl) methanamine hydro chloride, (R)-2-(2-((3-(aminomethyl)pyrrolidin-1-yl)methyl)-4-chlorophenoxy)ethan-1-ol hydrochloride, (S)-2-(2-((3-(aminomethyl)pyrrolidin-1-yl)methyl)-4-chlorophenoxy)ethan-1-ol hydrochloride, (R)-(1-(2-butoxy-5-chlorobenzyl)pyrrolidin-3-yl)methanamine hydrochloride, (S)-(1-(2-butoxy-5-chlorobenzyl)pyrrolidin-3-yl)methanamine hydrochloride, (S)-(4-(5-chloro-2-ethoxybenzyl)morpholin-2-yl)methanamine hydrochloride, (R)-(4-(5-chloro-2-ethoxybenzyl)morpholin-2-yl)methanamine hydrochloride, (S)-(1-(3-chloro-4-ethoxybenzyl)pyrrolidin-3-yl)methanamine hydrochloride, (S)-(1-(3-chloro-5-ethoxybenzyl)pyrrolidin-3-yl)methanamine hydro chloride, (S)-3-methyl-1-(2-propoxy-5-(trifluoromethoxy)benzyl) piperazine hydrochloride, (S)-(1-(2-(6-chlorobenzo[d][1,3]dioxol-4-yl)ethyl)pyrrolidin-3-yl)methanamine hydrochloride, (S)-(1-(2,3-dichloro-6-ethoxyphenethyl)pyrrolidin-3-yl)methanamine hydrochloride, (S)-(1-(4,5-dichloro-2-ethoxyphenethyl)pyrrolidin-3-yl)methanamine hydrochloride, (R)-1-(2-ethoxy-5-(trifluoromethyl)benzyl)-3-methylpiperazine hydrochloride, (S)-1-(4,5-dichloro-2-ethoxybenzyl)-3-methylpiperazine hydrochloride, (S)-1-(2-ethoxy-5-(trifluoromethoxy)benzyl)-3-methylpiperazine hydrochloride, (1-(3-chloro-4-ethoxybenzyl)pyrrolidin-3-yl)methanamine hydrochloride, 1-(5-chloro-2-ethoxybenzyl)piperazine hydrochloride, (4-(5-chloro-2-ethoxybenzyl)morpholin-2-yl)methanamine hydrochloride, (S)-(1-(2-ethoxy-5-(trifluoromethyl)phenethyl)pyrrolidin-3-yl)methanamine hydrochloride, (S)-(1-(5-chloro-2-propoxyphenethyl)piperidin-3-yl)methanamine hydrochloride, (S)-(1-(5-chloro-2-(cyclopropylmethoxy)phenethyl)pyrrolidin-3-yl)methanamine hydrochloride, (S)-(1-(5-chloro-2-ethoxyphenethyl)piperidin-3-yl)methanamine hydrochloride, (S)-1-(5-chloro-2-(cyclopropylmethoxy)benzyl)-3-methylpiperazine hydrochloride, (S)-1-(5-chloro-2-propoxybenzyl)-3-methylpiperazine hydrochloride, (S)-(1-(5-chloro-2-propoxyphenethyl)pyrrolidin-3-yl)methanamine hydrochloride, (S)-(1-(5-chloro-2-ethoxyphenethyl)pyrrolidin-3-yl)methanamine hydrochloride, (S)-1-(1-(5-chloro-2-ethoxybenzyl)pyrrolidin-3-yl)-N,N-dimethyl methanamine, (S)-1-(1-(5-chloro-2-ethoxybenzyl)pyrrolidin-3-yl)-N-methylmethanamine, (S)-(1-(5-chloro-2-isobutoxybenzyl)pyrrolidin-3-yl)methanamine hydrochloride, (S)-(1-(5-chloro-2-isopropoxybenzyl)pyrrolidin-3-yl)methanamine hydrochloride, (S)-(1-(5-chloro-2-(2-methoxyethoxy)benzyl)pyrrolidin-3-yl)methanamine hydrochloride, (S)-(1-((6-chlorobenzo[d][1,3]dioxol-4-yl)methyl)pyrrolidin-3-yl)methanamine hydrochloride, (R)-(1-(5-chloro-2-ethoxybenzyl)pyrrolidin-3-yl)methanamine hydrochloride, (S)-(1-(3-chloro-4-ethoxy-2-fluorobenzyl)pyrrolidin-3-yl)methanamine hydrochloride, (S)-(1-(5-chloro-4-ethoxy-2-fluorobenzyl)pyrrolidin-3-yl)methanamine hydrochloride, (R)-4-((3-(aminomethyl)pyrrolidin-1-yl)methyl)-2-chloro-5-ethoxy benzonitrile hydrochloride, (S)-4-((3-(aminomethyl)pyrrolidin-1-yl)methyl)-2-chloro-5-ethoxybenzonitrile hydrochloride, (S)-(4-(5-chloro-2-ethoxyphenethyl)morpholin-2-yl)methanamine hydrochloride, (S)-3-(2-(3-(aminomethyl)piperidin-1-yl)ethyl)-4-ethoxybenzonitrile hydrochloride, (S)-3-(2-(3-(aminomethyl)pyrrolidin-1-yl)ethyl)-4-(cyclopropylmethoxy)benzonitrile hydrochloride, (S)-(1-(3-chloro-4-ethoxyphenethyl)piperidin-3-yl)methanamine hydrochloride, and (S)-1-(5-chloro-2-ethoxybenzyl)pyrrolidin-3-amine hydrochloride.

In another particularly preferred embodiment, the compound of formula (I) is a compound selected from the group consisting of
(S)-(1-(3,5-dichloro-4-ethoxybenzyl)pyrrolidin-3-yl)methanamine hydrochloride, 1-[2-(3-chloro-4-ethoxyphenyl)ethyl]-4-piperidylamine hydrochloride,
(S)-1-{2-[3,5-dichloro-4-(2-methoxyethoxy)phenyl]ethyl}-3-piperidylamine hydrochloride,
(R)-1-{[3,5-dichloro-4-(cyclopropylmethoxy)phenyl]methyl}-3-pyrrolidinylamine hydrochloride,
(S)-1-{2-[3,5-dichloro-4-(cyclopropylmethoxy)phenyl]ethyl}-3-piperidylamine hydrochloride,
1-[(3-chloro-4-ethoxyphenyl)methyl]-4-piperidylamine hydrochloride,
(R)-1-{2-[3,5-dichloro-4-(2-methoxyethoxy)phenyl]ethyl}-3-piperidylamine hydrochloride,
(S)-1-{[3,5-dichloro-4-(cyclopropylmethoxy)phenyl]methyl}-3-pyrrolidinylamine hydrochloride,
(R)-1-{2-[3,5-dichloro-4-(cyclopropylmethoxy)phenyl]ethyl}-3-piperidylamine hydrochloride,
(S)-1-{2-[3-chloro-4-(cyclopropylmethoxy)phenyl]ethyl}-3-piperidylamine formate,
(S)-1-{2-[3-chloro-4-(cyclopropylmethoxy)phenyl]ethyl}-3-piperidylamine hydrochloride,
(R)-1-{2-[3-chloro-4-(cyclopropylmethoxy)phenyl]ethyl}-3-piperidylamine formate,
(R)-1-{2-[3-chloro-4-(cyclopropylmethoxy)phenyl]ethyl}-3-piperidylamine hydrochloride,
1-{[3-chloro-4-(cyclopropylmethoxy)phenyl]methyl}-4-piperidylamine hydrochloride,
5-{[(R)-3-(Aminomethyl)-1-pyrrolidinyl]methyl}-3-chloro-2-ethoxybenzonitrile hydrochloride,
3-{2-[(S)-3-Amino-1-piperidyl]ethyl}-4-ethoxybenzonitrile hydrochloride,
3-{2-[(R)-3-Amino-1-piperidyl]ethyl}-4-ethoxybenzonitrile hydrochloride,
5-{2-[(S)-3-amino-1-pyrrolidinyl]ethyl}-2-ethoxybenzonitrile hydrochloride,
3-{2-[(S)-3-Amino-1-pyrrolidinyl]ethyl}-4-ethoxybenzonitrile hydrochloride,
3-{2-[(R)-3-amino-1-pyrrolidinyl]ethyl}-4-ethoxybenzonitrile hydrochloride,
(R)-1-[2-(4-ethoxy-3-fluorophenyl)ethyl]-3-pyrrolidinylamine hydrochloride,
(R)-1-{2-[3-chloro-4-(2-methoxyethoxy)phenyl]ethyl}-3-piperidylamine hydrochloride,
1-[(3,5-Dichloro-4-ethoxyphenyl)methyl]-4-piperidylamine hydrochloride,
1-{2-[3,5-Dichloro-4-(cyclopropylmethoxy)phenyl]ethyl}-4-piperidylamine hydrochloride,
(S)-1-[(3-chloro-4-ethoxyphenyl)methyl]-3-piperidylamine hydrochloride,
(R)-1-[(3-chloro-4-ethoxyphenyl)methyl]-3-piperidylamine hydrochloride,
1-{[3,5-dichloro-4-(2-methoxyethoxy)phenyl]methyl}-4-piperidylamine hydrochloride,
1-{[3-chloro-4-(2-methoxyethoxy)phenyl]methyl}-4-piperidylamine hydrochloride,
(S)-1-{[3,5-dichloro-4-(cyclopropylmethoxy)phenyl]methyl}-3-piperidylamine hydrochloride,
(R)-1-{[3,5-dichloro-4-(cyclopropylmethoxy)phenyl]methyl}-3-piperidylamine hydrochloride,
1-{[3,5-dichloro-4-(cyclopropylmethoxy)phenyl]methyl}-4-piperidylamine hydrochloride,
(R)-1-{2-[3-chloro-4-(cyclopropylmethoxy)phenyl]ethyl}-3-pyrrolidinylamine hydrochloride,
(S)-1-{2-[3-chloro-4-(cyclopropylmethoxy)phenyl]ethyl}-3-pyrrolidinylamine hydrochloride,
({(S)-1-[(3-chloro-5-ethoxyphenyl)methyl]-3-pyrrolidinyl}methyl)amine hydrochloride,
and ({1-[(5-chloro-2-ethoxyphenyl)methyl]-3-azetidinyl}methyl)amine hydrochloride.

In another particularly preferred embodiment, the compound of formula (I) is a compound selected from the group consisting of
(S)-1-(2-ethoxy-5-(trifluoromethyl)benzyl)-3-methylpiperazine,
(S)-(1-(2-ethoxy-5-(trifluoromethyl) benzyl) pyrrolidin-3-yl) methanamine,
(S)-(1-(5-chloro-2-propoxybenzyl) pyrrolidin-3-yl) methanamine,
(S)-(1-(4,5-dichloro-2-ethoxybenzyl)pyrrolidin-3-yl)methanamine,
(S)-(1-(4,5-dichloro-2-(cyclopropylmethoxy)benzyl)pyrrolidin-3-yl)methanamine,
(S)-(1-(5-chloro-2-ethoxybenzyl)piperidin-3-yl)methanamine,
(S)-(1-(5-chloro-2-(cyclopropylmethoxy)benzyl)piperidin-3-yl)methanamine,
(S)-1-(5-chloro-2-ethoxybenzyl)-3-methylpiperazine,
1-(5-chloro-2-ethoxybenzyl)-4-ethylpiperazine,
1-(5-chloro-2-ethoxybenzyl)-N,N-dimethylpiperidin-4-amine,
(S)-(1-(5-chloro-2-ethoxybenzyl)pyrrolidin-3-yl)methanamine,
(S)-(1-(5-chloro-2-ethoxybenzyl)pyrrolidin-2-yl)methanamine,
(R)-(1-(5-chloro-2-ethoxybenzyl)pyrrolidin-2-yl)methanamine,
(R)-1-(5-chloro-2-ethoxybenzyl)piperidin-3-amine,
(S)-1-(5-chloro-2-ethoxybenzyl)piperidin-3-amine,
(1-(5-chloro-2-ethoxybenzyl)azetidin-3-yl)methanamine,
1-(5-chloro-2-ethoxybenzyl)-4-isopropylpiperazine,
(S)-1-(5-chloro-2-ethoxyphenethyl)piperidin-3-amine,
(S)-(1-(3-chloro-4-(cyclopropylmethoxy)phenethyl)pyrrolidin-3-yl)methanamine,
(R)-(1-(2-ethoxy-4,5-difluorobenzyl)pyrrolidin-3-yl)methanamine,
(S)-(1-(2-ethoxy-4,5-difluorobenzyl)pyrrolidin-3-yl)methanamine,
(R)-(1-(3-chloro-4-ethoxybenzyl)pyrrolidin-3-yl) methanamine,
(R)-2-(2-((3-(aminomethyl)pyrrolidin-1-yl)methyl)-4-chlorophenoxy)ethan-1-ol,
(S)-2-(2-((3-(aminomethyl)pyrrolidin-1-yl)methyl)-4-chlorophenoxy)ethan-1-ol,
(R)-(1-(2-butoxy-5-chlorobenzyl)pyrrolidin-3-yl)methanamine,
(S)-(1-(2-butoxy-5-chlorobenzyl)pyrrolidin-3-yl)methanamine,
(S)-(4-(5-chloro-2-ethoxybenzyl)morpholin-2-yl)methanamine,
(R)-(4-(5-chloro-2-ethoxybenzyl)morpholin-2-yl)methanamine,
(S)-(1-(3-chloro-4-ethoxybenzyl)pyrrolidin-3-yl)methanamine,
(S)-(1-(3-chloro-5-ethoxybenzyl)pyrrolidin-3-yl)methanamine,
(S)-3-methyl-1-(2-propoxy-5-(trifluoromethoxy)benzyl) piperazine,
(S)-(1-(2-(6-chlorobenzo[d][1,3]dioxol-4-yl)ethyl)pyrrolidin-3-yl)methanamine,
(S)-(1-(2,3-dichloro-6-ethoxyphenethyl)pyrrolidin-3-yl)methanamine,
(S)-(1-(4,5-dichloro-2-ethoxyphenethyl)pyrrolidin-3-yl)methanamine,
(R)-1-(2-ethoxy-5-(trifluoromethyl)benzyl)-3-methylpiperazine,
(S)-1-(4,5-dichloro-2-ethoxybenzyl)-3-methylpiperazine,
(S)-1-(2-ethoxy-5-(trifluoromethoxy)benzyl)-3-methylpiperazine,
(1-(3-chloro-4-ethoxybenzyl)pyrrolidin-3-yl)methanamine,
1-(5-chloro-2-ethoxybenzyl)piperazine,
(4-(5-chloro-2-ethoxybenzyl)morpholin-2-yl)methanamine,
(S)-(1-(2-ethoxy-5-(trifluoromethyl)phenethyl)pyrrolidin-3-yl)methanamine,
(S)-(1-(5-chloro-2-propoxyphenethyl)piperidin-3-yl)methanamine,
(S)-(1-(5-chloro-2-(cyclopropylmethoxy)phenethyl)pyrrolidin-3-yl)methanamine,
(S)-(1-(5-chloro-2-ethoxyphenethyl)piperidin-3-yl)methanamine,
(S)-1-(5-chloro-2-(cyclopropylmethoxy)benzyl)-3-methylpiperazine,
(S)-1-(5-chloro-2-propoxybenzyl)-3-methylpiperazine,
(S)-(1-(5-chloro-2-propoxyphenethyl)pyrrolidin-3-yl)methanamine,
(S)-(1-(5-chloro-2-ethoxyphenethyl)pyrrolidin-3-yl)methanamine,
(S)-1-(1-(5-chloro-2-ethoxybenzyl)pyrrolidin-3-yl)-N,N-dimethyl methanamine,
(S)-1-(1-(5-chloro-2-ethoxybenzyl)pyrrolidin-3-yl)-N-methylmethanamine,
(S)-(1-(5-chloro-2-isobutoxybenzyl)pyrrolidin-3-yl)methanamine,
(S)-(1-(5-chloro-2-isopropoxybenzyl)pyrrolidin-3-yl)methanamine,
(S)-(1-(5-chloro-2-(2-methoxyethoxy)benzyl)pyrrolidin-3-yl)methanamine,
(S)-(1-((6-chlorobenzo[d][1,3]dioxol-4-yl)methyl)pyrrolidin-3-yl)methanamine,
(R)-(1-(5-chloro-2-ethoxybenzyl)pyrrolidin-3-yl)methanamine,
(S)-(1-(3-chloro-4-ethoxy-2-fluorobenzyl)pyrrolidin-3-yl)methanamine,
(S)-(1-(5-chloro-4-ethoxy-2-fluorobenzyl)pyrrolidin-3-yl)methanamine,
(R)-4-((3-(aminomethyl)pyrrolidin-1-yl)methyl)-2-chloro-5-ethoxy benzonitrile,
(S)-4-((3-(aminomethyl)pyrrolidin-1-yl)methyl)-2-chloro-5-ethoxybenzonitrile,
(S)-(4-(5-chloro-2-ethoxyphenethyl)morpholin-2-yl)methanamine,
(S)-3-(2-(3-(aminomethyl)piperidin-1-yl)ethyl)-4-ethoxybenzonitrile,
(S)-3-(2-(3-(aminomethyl)pyrrolidin-1-yl)ethyl)-4-(cyclopropylmethoxy)benzonitrile,
(S)-(1-(3-chloro-4-ethoxyphenethyl)piperidin-3-yl)methanamine,
(S)-1-(5-chloro-2-ethoxybenzyl)pyrrolidin-3-amine,
(S)-(1-(3,5-dichloro-4-ethoxybenzyl)pyrrolidin-3-yl)methanamine,
1-[2-(3-chloro-4-ethoxyphenyl)ethyl]-4-piperidylamine,
(S)-1-{2-[3,5-dichloro-4-(2-methoxyethoxy)phenyl]ethyl}-3-piperidylamine,
(R)-1-{[3,5-dichloro-4-(cyclopropylmethoxy)phenyl]methyl}-3-pyrrolidinylamine,
(S)-1-{2-[3,5-dichloro-4-(cyclopropylmethoxy)phenyl]ethyl}-3-piperidylamine,
1-[(3-chloro-4-ethoxyphenyl)methyl]-4-piperidylamine,
(R)-1-{2-[3,5-dichloro-4-(2-methoxyethoxy)phenyl]ethyl}-3-piperidylamine,
(S)-1-{[3,5-dichloro-4-(cyclopropylmethoxy)phenyl]methyl}-3-pyrrolidinylamine,
(R)-1-{2-[3,5-dichloro-4-(cyclopropylmethoxy)phenyl]ethyl}-3-piperidylamine,
(S)-1-{2-[3-chloro-4-(cyclopropylmethoxy)phenyl]ethyl}-3-piperidylamine,
(R)-1-{2-[3-chloro-4-(cyclopropylmethoxy)phenyl]ethyl}-3-piperidylamine,
1-{[3-chloro-4-(cyclopropylmethoxy)phenyl]methyl}-4-piperidylamine,
5-{[(R)-3-(Aminomethyl)-1-pyrrolidinyl]methyl}-3-chloro-2-ethoxybenzonitrile,
3-{2-[(S)-3-Amino-1-piperidyl]ethyl}-4-ethoxybenzonitrile,
3-{2-[(R)-3-Amino-1-piperidyl]ethyl}-4-ethoxybenzonitrile,
5-{2-[(S)-3-amino-1-pyrrolidinyl]ethyl}-2-ethoxybenzonitrile,
3-{2-[(S)-3-Amino-1-pyrrolidinyl]ethyl}-4-ethoxybenzonitrile,
3-{2-[(R)-3-amino-1-pyrrolidinyl]ethyl}-4-ethoxybenzonitrile,
(R)-1-[2-(4-ethoxy-3-fluorophenyl)ethyl]-3-pyrrolidinylamine,
(R)-1-{2-[3-chloro-4-(2-methoxyethoxy)phenyl]ethyl}-3-piperidylamine,
1-[(3,5-Dichloro-4-ethoxyphenyl)methyl]-4-piperidylamine,
1-{2-[3,5-Dichloro-4-(cyclopropylmethoxy)phenyl]ethyl}-4-piperidylamine,
(S)-1-[(3-chloro-4-ethoxyphenyl)methyl]-3-piperidylamine,
(R)-1-[(3-chloro-4-ethoxyphenyl)methyl]-3-piperidylamine,
1-{[3,5-dichloro-4-(2-methoxyethoxy)phenyl]methyl}-4-piperidylamine,
1-{[3-chloro-4-(2-methoxyethoxy)phenyl]methyl}-4-piperidylamine,
(S)-1-{[3,5-dichloro-4-(cyclopropylmethoxy)phenyl]methyl}-3-piperidylamine,
(R)-1-{[3,5-dichloro-4-(cyclopropylmethoxy)phenyl]methyl}-3-piperidylamine,
1-{[3,5-dichloro-4-(cyclopropylmethoxy)phenyl]methyl}-4-piperidylamine,
(R)-1-{2-[3-chloro-4-(cyclopropylmethoxy)phenyl]ethyl}-3-pyrrolidinylamine,
(S)-1-{2-[3-chloro-4-(cyclopropylmethoxy)phenyl]ethyl}-3-pyrrolidinylamine,
({(S)-1-[(3-chloro-5-ethoxyphenyl)methyl]-3-pyrrolidinyl}methyl)amine, and
({1-[(5-chloro-2-ethoxyphenyl)methyl]-3-azetidinyl}methyl)amine.

Also provided herein is a compound as shown in Table 1.

### Definitions

The term “compound(s) of the present invention” is to be understood as equivalent to the term “compound(s) according to the invention”, and also covers a salt, stereoisomer, tautomer or N-oxide thereof. Pharmaceutically acceptable salts, solvates, stereoisomers, tautomers or N-oxides are also covered. The compounds according to the invention may be amorphous or may exist in one or more different crystalline states (polymorphs), which may have different macroscopic properties such as stability or show different biological properties such as activities. The present invention relates to amorphous and crystalline forms of the compounds of formula (I), mixtures of different crystalline states of the compounds of formula (I), as well as amorphous or crystalline salts thereof.

Salts of the compounds according to the invention are preferably pharmaceutically acceptable salts, such as those containing counterions present in drug products listed in the US FDA Orange Book database. They can be formed in a customary manner, e.g., by reacting the compound with an acid of the anion in question, if the compounds according to the invention have a basic functionality, or by reacting acidic compounds according to the invention with a suitable base. This may include addition salts of inorganic acids such as hydrochloride, hydrobromide, hydroiodide, sulphate, phosphate, diphosphate and nitrate or of organic acids such as acetate, maleate, fumarate, tartrate, succinate, citrate, lactate, methanesulphonate, p-toluenesulphonate, palmoate and stearate. Exemplary salts also include oxalate, chloride, bromide, iodide, bisulphate, acid phosphate, isonicotinate, salicylate, acid citrate, oleate, tannate, pantothenate, bitartrate, ascorbate, gentisinate, gluconate, glucuronate, saccharate, formate, benzoate, glutamate, ethanesulfonate, and benzenesulfonate salts.

The compounds of the present invention can also be present in the form of the free base. The term “free base”, as used herein, refers to the neutral form of the compounds of the present invention, i.e., the compounds not in the form of a salt. The free base of the compounds of the present invention can be formed by common methods known to the person skilled in the art. For example, if the synthesis of the compounds according to the present invention provides the product compounds in the form of a salt, in particular a salt of an inorganic or organic acid (such as the hydrochloride salt), the free base can be obtained, e.g., by treating the salt of an inorganic or organic acid, such as the hydrochloride salt, with an alkaline aqueous solution (such as, e.g., sodium hydroxide or ammonia) and extracting said solution with an organic non-polar solvent (such as, e.g., diethyl ether or DCM). The organic solvent phase containing the free base can then be separated followed by evaporation of the organic solvent. For example, the free base of the compounds of the present invention can be obtained following the experimental procedure for salt breaking described below.

Depending on the substitution pattern, the compounds according to the invention may have one or more centres of chirality, including axial chirality. The invention provides both, pure enantiomers or pure diastereomers of the compounds according to the invention, and their mixtures, including racemic mixtures. Suitable compounds according to the invention also include all possible geometrical stereoisomers (cis/trans isomers or E/Z isomers) and mixtures thereof. E/Z-isomers may be present with respect to, e.g., an alkene, carbon-nitrogen double-bond or amide group.

Tautomers may be formed, if a substituent is present at the compound of formula (I) of the present invention, which allows for the formation of tautomers such as keto-enol tautomers, imine-enamine tautomers, amide-imidic acid tautomers or the like.

The term “N-oxide” includes any compound of the present invention, which has at least one tertiary nitrogen atom that is oxidized to a N-oxide moiety.

The term “substituted”, as used herein, means that a hydrogen atom bonded to a designated atom is replaced with a specified substituent, provided that the substitution results in a stable or chemically feasible compound. Unless otherwise indicated, a substituted atom may have one or more substituents and each substituent is independently selected.

The term “substitutable”, when used in reference to a designated atom, means that attached to the atom is a hydrogen, which can be replaced with a suitable substituent.

When it is referred to certain atoms or moieties being substituted with “one or more” substituents, the term “one or more” is intended to cover at least one substituent, e.g. 1, 2, 3, 4, or 5 substituents, preferably 1, 2, or 3 substituents, more preferably 1, or 2 substituents. When neither the term “unsubstituted” nor “substituted” is explicitly mentioned concerning a moiety, said moiety is to be considered as unsubstituted.

The organic moieties mentioned in the above definitions of the variables are—like the term halogen—collective terms for individual listings of the individual group members. The prefix Cₙ-Cₘ indicates in each case the possible number of carbon atoms in the group.

The term “halogen” denotes in each case fluorine, bromine, chlorine or iodine, in particular fluorine, chlorine, or bromine, preferably fluorine or chlorine.

The term “alkyl” as used herein denotes in each case a straight-chain or branched alkyl group having usually from 1 to 6 carbon atoms, preferably 1 to 5 or 1 to 4 carbon atoms, more preferably 1 to 3 or 1 or 2 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, and 1-ethylpropyl.

The term “haloalkyl” as used herein denotes in each case a straight-chain or branched alkyl group having usually from 1 to 4 carbon atoms, preferably 1 to 3 or 1 or 2 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “alkoxy” as used herein denotes in each case a straight-chain or branched alkyl group which is bonded via an oxygen atom and has usually from 1 to 6 carbon atoms, preferably 1 to 2 carbon atoms, more preferably 2 carbon atoms. Examples of an alkoxy group are methoxy, ethoxy, n-propoxy, iso-propoxy, n-butyloxy, 2-butyloxy, iso-butyloxy, tert.-butyloxy, and the like.

The term “haloalkoxy” as used herein denotes in each case a straight-chain or branched alkoxy group having from 1 to 6 carbon atoms, preferably 1 to 2 carbon atoms, more preferably 2 carbon atoms, wherein the hydrogen atoms of this group are partially or totally replaced with halogen atoms, in particular fluorine atoms. Preferred haloalkoxy moieties include C₂-haloalkoxy, in particular C₂-fluoroalkoxy, such as trifluoroethoxy and the like.

The term “cycloalkyl” as used herein denotes in each case a monocyclic cycloaliphatic radical having usually from 3 to 6 carbon atoms, such as cyclopropyl, cyclobutyl, cyclopentyl, or cyclohexyl.

The term “carbocyclic” or “carbocyclyl” includes, unless otherwise indicated, in general a 3- to 9-membered, preferably a 3- to 6-membered, more preferably a 5- or 6-membered monocyclic ring comprising 3 to 9, preferably 3 to 6, more preferably 5 or 6 carbon atoms.

The carbocycle may be saturated, partially or fully unsaturated, or aromatic, wherein saturated means that only single bonds are present, and partially or fully unsaturated means that one or more double bonds may be present in suitable positions, while the Hückel rule for aromaticity is not fulfilled, whereas aromatic means that the Hückel (4n+2) rule is fulfilled. The term “carbocycle” or “carbocyclyl”, unless otherwise indicated, may therefore cover inter alia cycloalkyl, cycloalkenyl, as well as phenyl. Preferably, the term “carbocycle” covers cycloalkyl and cycloalkenyl groups, for example cyclopropane, cyclobutane, cyclopentane and cyclohexane rings.

The term “heterocyclic” or “heterocyclyl” includes, unless otherwise indicated, in general a 3- to 9-membered, preferably a 3- to 6-membered, more preferably 5- or 6-membered monocyclic ring. The heterocycle may be saturated, partially or fully unsaturated, or aromatic, wherein saturated means that only single bonds are present, and partially or fully unsaturated means that one or more double bonds may be present in suitable positions, while the Hückel rule for aromaticity is not fulfilled, whereas aromatic means that the Hückel (4n+2) rule is fulfilled. The heterocycle typically comprises one or more, e.g. 1, 2, or 3, preferably 1, or 2 heteroatoms selected from N, O and S as ring members, where S-atoms as ring members may be present as S, SO or SO₂. The remaining ring members are carbon atoms. The saturated or partially or fully unsaturated heterocycles usually comprise 1, 2, or 3, preferably 1, or 2 heteroatoms selected from N, O and S as ring members, where S-atoms as ring members may be present as S, SO or SO₂. The skilled person is aware that S, SO or SO₂ is to be understood as follows:

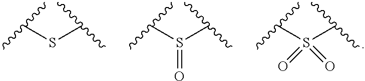

Further, a skilled person is aware that resonance structures of the oxidized forms may be possible. Saturated heterocycles include, unless otherwise indicated, in general 3- to 9-membered, preferably 3- to 6-membered, more preferably 5- or 6-membered monocyclic rings comprising 3 to 9, preferably 3 to 6, more preferably 5 or 6 atoms comprising at least one heteroatom, such as pyrrolidine, tetrahydrothiophene, tetrahydrofuran, piperidine, tetrahydropyran, dioxane, morpholine or piperazine.

As used herein, the terms “carbocyclylalkyl” and “heteroocyclylalkyl” and the like refer to the corresponding groups, which are bonded to the remainder of the molecule via an alkyl, preferably via a C₁-C₂-alkyl group. Preferred examples include benzyl (i.e. phenylmethyl).

As used herein, the term “alkylene” refers to a linking straight-chain or branched alkylene group having usually from 1 to 4 carbon atoms, e.g. 1, 2, 3, or 4 carbon atoms. The alkylene group bridges a certain group to the remainder of the molecule. Preferred alkylene groups include methylene (CH₂), ethylene (CH₂CH₂), propylene (CH₂CH₂CH₂) and the like. A skilled person understands that, if it is referred, e.g., to CH₂ that the carbon atom being tetravalent has two valences left for forming a bridge (—CH₂—). Similarly, when it is referred, e.g., to CH₂CH₂, each carbon atom has one valence left for forming a bridge (—CH₂CH₂—). Furthermore, when is it referred, e.g., to CH₂CH₂CH₂, each terminal carbon atom has one valence left for forming a bridge (—CH₂CH₂CH₂—).

As used in the specification and the claims, the singular forms of “a” and “an” also include the corresponding plurals unless the context clearly dictates otherwise. The same applies for plural forms used herein, which also include the singular forms unless the context clearly dictates otherwise.

The terms “about” and “approximately” in the context of the present invention denotes an interval of accuracy that a person skilled in the art will understand to still ensure the technical effect of the feature in question. The term typically indicates a deviation from the indicated numerical value of 10% and preferably +5%.

It needs to be understood that the term “comprising” is not limiting. For the purposes of the present invention, the term “consisting of” is considered to be a preferred embodiment of the term “comprising of”. If hereinafter a group is defined to comprise at least a certain number of embodiments, this is also meant to encompass a group, which preferably consists of these embodiments only.

The term “treatment” is to be understood as also including the option of “prophylaxis”. Thus, whenever reference is made herein to a “treatment” or “treating”, this is to be understood as “treatment and/or prophylaxis” or “treating and/or preventing”.

The term “pharmaceutically acceptable excipient, carrier or diluent” refers to a solid or liquid filler, diluent or encapsulating substance, which does not interfere with the effectiveness or the biological activity of the active ingredients and which is not toxic to the host, which may be either humans or animals, to which it is administered. Depending upon the particular route of administration, a variety of pharmaceutically-acceptable carriers such as those well known in the art may be used. Non-limiting examples include sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water. Pharmaceutically acceptable carriers or excipients also include diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO₂), solvents/co-solvents (e.g. aqueous vehicle, propylene glycol, glycerol), buffering agents (e.g. citrate, gluconates, lactates), preservatives (e.g. Na benzoate, parabens (Me, Pr and Bu), BKC), anti-oxidants (e.g. BHT, BHA, ascorbic acid), wetting agents (e.g. polysorbates, sorbitan esters), thickening agents (e.g. methylcellulose or hydroxyethylcellulose), sweetening agents (e.g. sorbitol, saccharin, aspartame, acesulfame), flavouring agents (e.g. peppermint, lemon oils, butterscotch, etc.), humectants (e.g. propylene glycol, glycerol, sorbitol).

The term “mammal” as used herein includes a mouse, rat, monkey, cat, dog, rabbit, goat, sheep, horse, camel, lama, cow and a human.

The terms “subject” and “patient” are used interchangeably and refer to a mammal or a human.

The term “therapeutically effective amount” refer to a predetermined amount of the compound or pharmaceutical composition which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect or physician observes a change).

### Description of Pharmaceutical Compositions, Medical Uses and Uses According to the Present Invention

The present invention also provides a pharmaceutical composition comprising the compound according to formula (I) and optionally a pharmaceutically acceptable excipient or carrier.

The pharmaceutical composition may be formulated for any suitable route of administration including oral, subcutaneous, intravenous, intravenous or epidural patient controlled analgesia (PCA and PCEA), intramuscular, intrathecal, epidural, intracistemal, intraperitoneal, transdermal, topical, buccal, sublingual, transmucosal, inhalation, intra-articular, intranasal, rectal or ocular administration. The pharmaceutical composition may be formulated as a sustained release or controlled release formulation.

The present invention also provides the compound according to formula (I) for use in medicine. In one embodiment, the compound according to formula (I) is for use in the treatment of an autophagy-related disease or condition. In one embodiment, the compound according to formula (I) is for use in the treatment of cancer, age-related diseases, and infections.

In one embodiment, the compound according to formula (I) is for use in the treatment of a disease or condition selected from the group consisting of neurodegenerative diseases, Huntington's disease, Alzheimer's disease, Parkinson's disease, systemic lupus erythematosus, epilepsy, cancer, liver diseases (e.g. nonalcoholic fatty liver disease (NAFLD)), al antitrypsin deficiency, Charcot Marie Tooth syndrome, Rett Syndrome, Sickle Cell disease, Wilson Disease, amyloidosis, Gaucher's diseases, lysosomal and glycogen storage disorders (e.g., Glycogen Storage Disease type 1A (GSD1A)), cystic fibrosis; viral infection and diseases human cytomegalovirus (HCMV) infection, hepatitis B, human immunodeficiency virus infection, Zika virus infection, coronavirus infection, HCoV-229E, HCoV-NL63, betacoronavirus infection, such as HCoV-OC43, SARS-CoV-1, HCoV-HKU1, MERS-CoV or SARS-CoV-2, bacterial infections, metabolic disorders, diabetes, fibrosis, silicosis, diabetic retinopathy, glaucoma, cataracts, age-related macular degeneration, glomerulonephritis, glomerulosclerosis, wound healing disorders, Niemann-Pick type C (NPC) disease, fibrinogen storage disease (FSB), inclusion body disease (IBD), muscular dystrophy, Duchenne muscular dystrophy, Limb-girdle muscular dystrophy, myopathy, myofibrillar myopathy, hereditary myopathy, diabetic cardiomyopathy, anti-inflammatory disorders, autoimmune diseases, multiple sclerosis, rheumatoid arthritis, irritable bowel syndrome, Crohn's disease, vascular disorders, stroke, coronary artery diseases, myocardial infarction, unstable angina pectoris, atherosclerosis or vasculitis, Behcet's syndrome, giant cell arteritis, polymyalgia rheumatic, Wegener's granulomatosis, Churg-Strauss syndrome, vasculitis, Henoch-Schonlein pruprua, Kawasaki disease, viral infection or replication, pox virus infection, herpes virus infection, asthma, allergic rhinitis, COPD, osteoporosis, organ transplant rejection, psoriasis, hypertrophic scarring (keloid formation), adhesion formations following general or gynecological surgery, lung fibrosis, liver fibrosis, kidney fibrosis, disorders caused by intracellular parasites, malaria, tuberculosis, neuropathic pain, post-operative phantom limb pain or postherpetic neuralgia, allergies, amyotrophic lateral sclerosis (ALS), antigen induced recall response, immune response suppression, muscle degeneration and atrophy, frailty in aging, spinal cord injury, and diseases and conditions involving misfolded and/or nonfolded proteins.

In one embodiment, the compounds provided herein are for use in the treatment of an autophagy-related disease selected from the group consisting of neurodegenerative diseases, Huntington's disease, Alzheimer's disease, Parkinson's disease, liver diseases (e.g. nonalcoholic fatty liver disease (NAFLD)), al antitrypsin deficiency, Charcot Marie Tooth syndrome, Rett Syndrome, Wilson Disease, amyloidosis, Gaucher's diseases, lysosomal and glycogen storage disorders (e.g., Glycogen Storage Disease type 1A (GSD1A)), cystic fibrosis; viral infection and diseases human cytomegalovirus (HCMV) infection, hepatitis B, human immunodeficiency virus infection, Zika virus infection, coronavirus infection, HCoV-229E, HCoV-NL63, betacoronavirus infection, such as HCoV-OC43, SARS-CoV-1, HCoV-HKU1, MERS-CoV or SARS-CoV-2, fibrosis, silicosis, diabetic retinopathy, glaucoma, cataracts, age-related macular degeneration, glomerulonephritis, glomerulosclerosis, wound healing disorders, Niemann-Pick type C (NPC) disease, inclusion body disease (IBD), muscular dystrophy, Duchenne muscular dystrophy, Limb-girdle muscular dystrophy, myopathy, myofibrillar myopathy, hereditary myopathy, diabetic cardiomyopathy, anti-inflammatory disorders, autoimmune diseases, multiple sclerosis, rheumatoid arthritis, irritable bowel syndrome, Crohn's disease, vascular disorders, stroke, coronary artery diseases, myocardial infarction, unstable angina pectoris, atherosclerosis or vasculitis, Behcet's syndrome, viral infection or replication, pox virus infection, herpes virus infection, lung fibrosis, liver fibrosis, kidney fibrosis, disorders caused by intracellular parasites, malaria, neuropathic pain, allergies, amyotrophic lateral sclerosis (ALS), immune response suppression, muscle degeneration and atrophy, frailty in aging, spinal cord injury, and diseases and conditions involving misfolded and/or nonfolded proteins.

In a preferred embodiment, the compound according to formula (I) is for use in the treatment of a neurodegenerative disease. In a preferred embodiment, the neurodegenerative disease is Alzheimer's disease. In another preferred embodiment, the neurodegenerative disease is Parkinson's disease. In yet another preferred embodiment, the neurodegenerative disease is amyotrophic lateral sclerosis (ALS). In yet another preferred embodiment, the neurodegenerative disease is Huntington's disease.

In one embodiment, the compound of formula (I) is selected from the group consisting of (S)-1-(2-ethoxy-5-(trifluoromethyl)benzyl)-3-methylpiperazine hydrochloride, (S)-(1-(2-ethoxy-5-(trifluoromethyl) benzyl) pyrrolidin-3-yl) methanamine hydrochloride, (S)-(1-(5-chloro-2-propoxybenzyl) pyrrolidin-3-yl) methanamine Hydrochloride, (S)-(1-(5-chloro-2-(cyclopropylmethoxy)benzyl)pyrrolidin-3-yl)methanamine Hydrochloride, (S)-(1-(4,5-dichloro-2-ethoxybenzyl)pyrrolidin-3-yl)methanamine hydrochloride, (S)-(1-(4,5-dichloro-2-(cyclopropylmethoxy)benzyl)pyrrolidin-3-yl)methanamine hydrochloride, (S)-(1-(5-chloro-2-ethoxybenzyl)piperidin-3-yl)methanamine hydrochloride, (S)-(1-(5-chloro-2-(cyclopropylmethoxy)benzyl)piperidin-3-yl)methanamine hydrochloride, (S)-1-(5-chloro-2-ethoxybenzyl)-3-methylpiperazine hydrochloride, 1-(5-chloro-2-ethoxybenzyl)-4-ethylpiperazine, 1-(5-chloro-2-ethoxybenzyl)-N,N-dimethylpiperidin-4-amine, (S)-(1-(5-chloro-2-ethoxybenzyl)pyrrolidin-3-yl)methanamine hydrochloride, (S)-(1-(5-chloro-2-ethoxybenzyl)pyrrolidin-2-yl)methanamine Hydrochloride, (R)-(1-(5-chloro-2-ethoxybenzyl)pyrrolidin-2-yl)methanamine hydrochloride, (R)-1-(5-chloro-2-ethoxybenzyl)piperidin-3-amine hydrochloride, (S)-1-(5-chloro-2-ethoxybenzyl)piperidin-3-amine hydrochloride, (1-(5-chloro-2-ethoxybenzyl)azetidin-3-yl)methanamine Hydrochloride, and 1-(5-chloro-2-ethoxybenzyl)-4-isopropylpiperazine.

In another embodiment, the compound of formula (I) is selected from the group consisting of (S)-1-(5-chloro-2-ethoxyphenethyl)piperidin-3-amine hydrochloride,
(S)-(1-(3-chloro-4-(cyclopropylmethoxy)phenethyl)pyrrolidin-3-yl)methanamine hydrochloride,
(R)-(1-(2-ethoxy-4,5-difluorobenzyl)pyrrolidin-3-yl)methanamine hydrochloride,
(S)-(1-(2-ethoxy-4,5-difluorobenzyl)pyrrolidin-3-yl)methanamine hydrochloride,
(R)-(1-(3-chloro-4-ethoxybenzyl)pyrrolidin-3-yl) methanamine hydro chloride,
(R)-2-(2-((3-(aminomethyl)pyrrolidin-1-yl)methyl)-4-chlorophenoxy)ethan-1-ol hydrochloride,
(S)-2-(2-((3-(aminomethyl)pyrrolidin-1-yl)methyl)-4-chlorophenoxy)ethan-1-ol hydrochloride,
(R)-(1-(2-butoxy-5-chlorobenzyl)pyrrolidin-3-yl)methanamine hydrochloride,
(S)-(1-(2-butoxy-5-chlorobenzyl)pyrrolidin-3-yl)methanamine hydrochloride,
(S)-(4-(5-chloro-2-ethoxybenzyl)morpholin-2-yl)methanamine hydrochloride,
(R)-(4-(5-chloro-2-ethoxybenzyl)morpholin-2-yl)methanamine hydrochloride,
(S)-(1-(3-chloro-4-ethoxybenzyl)pyrrolidin-3-yl)methanamine hydrochloride,
(S)-(1-(3-chloro-5-ethoxybenzyl)pyrrolidin-3-yl)methanamine hydro chloride,
(S)-3-methyl-1-(2-propoxy-5-(trifluoromethoxy)benzyl) piperazine hydrochloride,
(S)-(1-(2-(6-chlorobenzo[d][1,3]dioxol-4-yl)ethyl)pyrrolidin-3-yl)methanamine hydrochloride,
(S)-(1-(2,3-dichloro-6-ethoxyphenethyl)pyrrolidin-3-yl)methanamine hydrochloride,
(S)-(1-(4,5-dichloro-2-ethoxyphenethyl)pyrrolidin-3-yl)methanamine hydrochloride,
(R)-1-(2-ethoxy-5-(trifluoromethyl)benzyl)-3-methylpiperazine hydrochloride,
(S)-1-(4,5-dichloro-2-ethoxybenzyl)-3-methylpiperazine hydrochloride,
(S)-1-(2-ethoxy-5-(trifluoromethoxy)benzyl)-3-methylpiperazine hydrochloride,
(1-(3-chloro-4-ethoxybenzyl)pyrrolidin-3-yl)methanamine hydrochloride,
1-(5-chloro-2-ethoxybenzyl)piperazine hydrochloride,
(4-(5-chloro-2-ethoxybenzyl)morpholin-2-yl)methanamine hydrochloride,
(S)-(1-(2-ethoxy-5-(trifluoromethyl)phenethyl)pyrrolidin-3-yl)methanamine hydrochloride,
(S)-(1-(5-chloro-2-propoxyphenethyl)piperidin-3-yl)methanamine hydrochloride,
(S)-(1-(5-chloro-2-(cyclopropylmethoxy)phenethyl)pyrrolidin-3-yl)methanamine hydrochloride,
(S)-(1-(5-chloro-2-ethoxyphenethyl)piperidin-3-yl)methanamine hydrochloride,
(S)-1-(5-chloro-2-(cyclopropylmethoxy)benzyl)-3-methylpiperazine hydrochloride,
(S)-1-(5-chloro-2-propoxybenzyl)-3-methylpiperazine hydrochloride,
(S)-(1-(5-chloro-2-propoxyphenethyl)pyrrolidin-3-yl)methanamine hydrochloride,
(S)-(1-(5-chloro-2-ethoxyphenethyl)pyrrolidin-3-yl)methanamine hydrochloride,
(S)-1-(1-(5-chloro-2-ethoxybenzyl)pyrrolidin-3-yl)-N,N-dimethyl methanamine,
(S)-1-(1-(5-chloro-2-ethoxybenzyl)pyrrolidin-3-yl)-N-methylmethanamine,
(S)-(1-(5-chloro-2-isobutoxybenzyl)pyrrolidin-3-yl)methanamine hydrochloride,
(S)-(1-(5-chloro-2-isopropoxybenzyl)pyrrolidin-3-yl)methanamine hydrochloride,
(S)-(1-(5-chloro-2-(2-methoxyethoxy)benzyl)pyrrolidin-3-yl)methanamine hydrochloride,
(S)-(1-((6-chlorobenzo[d][1,3]dioxol-4-yl)methyl)pyrrolidin-3-yl)methanamine hydrochloride,
(R)-(1-(5-chloro-2-ethoxybenzyl)pyrrolidin-3-yl)methanamine hydrochloride,
(S)-(1-(3-chloro-4-ethoxy-2-fluorobenzyl)pyrrolidin-3-yl)methanamine hydrochloride,
(S)-(1-(5-chloro-4-ethoxy-2-fluorobenzyl)pyrrolidin-3-yl)methanamine hydrochloride,
(R)-4-((3-(aminomethyl)pyrrolidin-1-yl)methyl)-2-chloro-5-ethoxy benzonitrile hydrochloride,
(S)-4-((3-(aminomethyl)pyrrolidin-1-yl)methyl)-2-chloro-5-ethoxybenzonitrile hydrochloride,
(S)-(4-(5-chloro-2-ethoxyphenethyl)morpholin-2-yl)methanamine hydrochloride,
(S)-3-(2-(3-(aminomethyl)piperidin-1-yl)ethyl)-4-ethoxybenzonitrile hydrochloride,
(S)-3-(2-(3-(aminomethyl)pyrrolidin-1-yl)ethyl)-4-(cyclopropylmethoxy)benzonitrile hydrochloride,
(S)-(1-(3-chloro-4-ethoxyphenethyl)piperidin-3-yl)methanamine hydrochloride, and
(S)-1-(5-chloro-2-ethoxybenzyl)pyrrolidin-3-amine hydrochloride.

In another embodiment, the compound of formula (I) is selected from the group consisting of (S)-1-(2-ethoxy-5-(trifluoromethyl)benzyl)-3-methylpiperazine hydrochloride, (S)-(1-(2-ethoxy-5-(trifluoromethyl) benzyl) pyrrolidin-3-yl) methanamine hydrochloride, (S)-(1-(5-chloro-2-propoxybenzyl) pyrrolidin-3-yl) methanamine Hydrochloride, (S)-(1-(5-chloro-2-(cyclopropylmethoxy)benzyl)pyrrolidin-3-yl)methanamine Hydrochloride, (S)-(1-(4,5-dichloro-2-ethoxybenzyl)pyrrolidin-3-yl)methanamine hydrochloride, (S)-(1-(4,5-dichloro-2-(cyclopropylmethoxy)benzyl)pyrrolidin-3-yl)methanamine hydrochloride, (S)-(1-(5-chloro-2-ethoxybenzyl)piperidin-3-yl)methanamine hydrochloride, (S)-(1-(5-chloro-2-(cyclopropylmethoxy)benzyl)piperidin-3-yl)methanamine hydrochloride, (S)-1-(5-chloro-2-ethoxybenzyl)-3-methylpiperazine hydrochloride, 1-(5-chloro-2-ethoxybenzyl)-4-ethylpiperazine, 1-(5-chloro-2-ethoxybenzyl)-N,N-dimethylpiperidin-4-amine, (S)-(1-(5-chloro-2-ethoxybenzyl)pyrrolidin-3-yl)methanamine hydrochloride, (S)-(1-(5-chloro-2-ethoxybenzyl)pyrrolidin-2-yl)methanamine Hydrochloride, (R)-(1-(5-chloro-2-ethoxybenzyl)pyrrolidin-2-yl)methanamine hydrochloride, (R)-1-(5-chloro-2-ethoxybenzyl)piperidin-3-amine hydrochloride, (S)-1-(5-chloro-2-ethoxybenzyl)piperidin-3-amine hydrochloride, (1-(5-chloro-2-ethoxybenzyl)azetidin-3-yl)methanamine Hydrochloride, 1-(5-chloro-2-ethoxybenzyl)-4-isopropylpiperazine, (S)-1-(5-chloro-2-ethoxyphenethyl)piperidin-3-amine hydrochloride, (S)-(1-(3-chloro-4-(cyclopropylmethoxy)phenethyl)pyrrolidin-3-yl)methanamine hydrochloride, (R)-(1-(2-ethoxy-4,5-difluorobenzyl)pyrrolidin-3-yl)methanamine hydrochloride, (S)-(1-(2-ethoxy-4,5-difluorobenzyl)pyrrolidin-3-yl)methanamine hydrochloride, (R)-(1-(3-chloro-4-ethoxybenzyl)pyrrolidin-3-yl) methanamine hydro chloride, (R)-2-(2-((3-(aminomethyl)pyrrolidin-1-yl)methyl)-4-chlorophenoxy)ethan-1-ol hydrochloride, (S)-2-(2-((3-(aminomethyl)pyrrolidin-1-yl)methyl)-4-chlorophenoxy)ethan-1-ol hydrochloride, (R)-(1-(2-butoxy-5-chlorobenzyl)pyrrolidin-3-yl)methanamine hydrochloride, (S)-(1-(2-butoxy-5-chlorobenzyl)pyrrolidin-3-yl)methanamine hydrochloride, (S)-(4-(5-chloro-2-ethoxybenzyl)morpholin-2-yl)methanamine hydrochloride, (R)-(4-(5-chloro-2-ethoxybenzyl)morpholin-2-yl)methanamine hydrochloride, (S)-(1-(3-chloro-4-ethoxybenzyl)pyrrolidin-3-yl)methanamine hydrochloride, (S)-(1-(3-chloro-5-ethoxybenzyl)pyrrolidin-3-yl)methanamine hydro chloride, (S)-3-methyl-1-(2-propoxy-5-(trifluoromethoxy)benzyl) piperazine hydrochloride, (S)-(1-(2-(6-chlorobenzo[d][1,3]dioxol-4-yl)ethyl)pyrrolidin-3-yl)methanamine hydrochloride, (S)-(1-(2,3-dichloro-6-ethoxyphenethyl)pyrrolidin-3-yl)methanamine hydrochloride, (S)-(1-(4,5-dichloro-2-ethoxyphenethyl)pyrrolidin-3-yl)methanamine hydrochloride, (R)-1-(2-ethoxy-5-(trifluoromethyl)benzyl)-3-methylpiperazine hydrochloride, (S)-1-(4,5-dichloro-2-ethoxybenzyl)-3-methylpiperazine hydrochloride, (S)-1-(2-ethoxy-5-(trifluoromethoxy)benzyl)-3-methylpiperazine hydrochloride, (1-(3-chloro-4-ethoxybenzyl)pyrrolidin-3-yl)methanamine hydrochloride, 1-(5-chloro-2-ethoxybenzyl)piperazine hydrochloride, (4-(5-chloro-2-ethoxybenzyl)morpholin-2-yl)methanamine hydrochloride, (S)-(1-(2-ethoxy-5-(trifluoromethyl)phenethyl)pyrrolidin-3-yl)methanamine hydrochloride, (S)-(1-(5-chloro-2-propoxyphenethyl)piperidin-3-yl)methanamine hydrochloride, (S)-(1-(5-chloro-2-(cyclopropylmethoxy)phenethyl)pyrrolidin-3-yl)methanamine hydrochloride, (S)-(1-(5-chloro-2-ethoxyphenethyl)piperidin-3-yl)methanamine hydrochloride, (S)-1-(5-chloro-2-(cyclopropylmethoxy)benzyl)-3-methylpiperazine hydrochloride, (S)-1-(5-chloro-2-propoxybenzyl)-3-methylpiperazine hydrochloride, (S)-(1-(5-chloro-2-propoxyphenethyl)pyrrolidin-3-yl)methanamine hydrochloride, (S)-(1-(5-chloro-2-ethoxyphenethyl)pyrrolidin-3-yl)methanamine hydrochloride, (S)-1-(1-(5-chloro-2-ethoxybenzyl)pyrrolidin-3-yl)-N,N-dimethyl methanamine, (S)-1-(1-(5-chloro-2-ethoxybenzyl)pyrrolidin-3-yl)-N-methylmethanamine, (S)-(1-(5-chloro-2-isobutoxybenzyl)pyrrolidin-3-yl)methanamine hydrochloride, (S)-(1-(5-chloro-2-isopropoxybenzyl)pyrrolidin-3-yl)methanamine hydrochloride, (S)-(1-(5-chloro-2-(2-methoxyethoxy)benzyl)pyrrolidin-3-yl)methanamine hydrochloride, (S)-(1-((6-chlorobenzo[d][1,3]dioxol-4-yl)methyl)pyrrolidin-3-yl)methanamine hydrochloride, (R)-(1-(5-chloro-2-ethoxybenzyl)pyrrolidin-3-yl)methanamine hydrochloride, (S)-(1-(3-chloro-4-ethoxy-2-fluorobenzyl)pyrrolidin-3-yl)methanamine hydrochloride, (S)-(1-(5-chloro-4-ethoxy-2-fluorobenzyl)pyrrolidin-3-yl)methanamine hydrochloride, (R)-4-((3-(aminomethyl)pyrrolidin-1-yl)methyl)-2-chloro-5-ethoxy benzonitrile hydrochloride, (S)-4-((3-(aminomethyl)pyrrolidin-1-yl)methyl)-2-chloro-5-ethoxybenzonitrile hydrochloride, (S)-(4-(5-chloro-2-ethoxyphenethyl)morpholin-2-yl)methanamine hydrochloride, (S)-3-(2-(3-(aminomethyl)piperidin-1-yl)ethyl)-4-ethoxybenzonitrile hydrochloride, (S)-3-(2-(3-(aminomethyl)pyrrolidin-1-yl)ethyl)-4-(cyclopropylmethoxy)benzonitrile hydrochloride, (S)-(1-(3-chloro-4-ethoxyphenethyl)piperidin-3-yl)methanamine hydrochloride, and (S)-1-(5-chloro-2-ethoxybenzyl)pyrrolidin-3-amine hydrochloride.

In another particularly preferred embodiment, the compound of formula (I) is a compound selected from the group consisting of
(S)-(1-(3,5-dichloro-4-ethoxybenzyl)pyrrolidin-3-yl)methanamine hydrochloride,
1-[2-(3-chloro-4-ethoxyphenyl)ethyl]-4-piperidylamine hydrochloride,
(S)-1-{2-[3,5-dichloro-4-(2-methoxyethoxy)phenyl]ethyl}-3-piperidylamine hydrochloride,
(R)-1-{[3,5-dichloro-4-(cyclopropylmethoxy)phenyl]methyl}-3-pyrrolidinylamine hydrochloride,
(S)-1-{2-[3,5-dichloro-4-(cyclopropylmethoxy)phenyl]ethyl}-3-piperidylamine hydrochloride,
1-[(3-chloro-4-ethoxyphenyl)methyl]-4-piperidylamine hydrochloride,
(R)-1-{2-[3,5-dichloro-4-(2-methoxyethoxy)phenyl]ethyl}-3-piperidylamine hydrochloride,
(S)-1-{[3,5-dichloro-4-(cyclopropylmethoxy)phenyl]methyl}-3-pyrrolidinylamine hydrochloride,
(R)-1-{2-[3,5-dichloro-4-(cyclopropylmethoxy)phenyl]ethyl}-3-piperidylamine hydrochloride,
(S)-1-{2-[3-chloro-4-(cyclopropylmethoxy)phenyl]ethyl}-3-piperidylamine formate,
(S)-1-{2-[3-chloro-4-(cyclopropylmethoxy)phenyl]ethyl}-3-piperidylamine hydrochloride,
(R)-1-{2-[3-chloro-4-(cyclopropylmethoxy)phenyl]ethyl}-3-piperidylamine formate,
(R)-1-{2-[3-chloro-4-(cyclopropylmethoxy)phenyl]ethyl}-3-piperidylamine hydrochloride,
1-{[3-chloro-4-(cyclopropylmethoxy)phenyl]methyl}-4-piperidylamine hydrochloride,
5-{[(R)-3-(Aminomethyl)-1-pyrrolidinyl]methyl}-3-chloro-2-ethoxybenzonitrile hydrochloride,
3-{2-[(S)-3-Amino-1-piperidyl]ethyl}-4-ethoxybenzonitrile hydrochloride,
3-{2-[(R)-3-Amino-1-piperidyl]ethyl}-4-ethoxybenzonitrile hydrochloride,
5-{2-[(S)-3-amino-1-pyrrolidinyl]ethyl}-2-ethoxybenzonitrile hydrochloride,
3-{2-[(S)-3-Amino-1-pyrrolidinyl]ethyl}-4-ethoxybenzonitrile hydrochloride,
3-{2-[(R)-3-amino-1-pyrrolidinyl]ethyl}-4-ethoxybenzonitrile hydrochloride,
(R)-1-[2-(4-ethoxy-3-fluorophenyl)ethyl]-3-pyrrolidinylamine hydrochloride,
(R)-1-{2-[3-chloro-4-(2-methoxyethoxy)phenyl]ethyl}-3-piperidylamine hydrochloride,
1-[(3,5-Dichloro-4-ethoxyphenyl)methyl]-4-piperidylamine hydrochloride,
1-{2-[3,5-Dichloro-4-(cyclopropylmethoxy)phenyl]ethyl}-4-piperidylamine hydrochloride,
(S)-1-[(3-chloro-4-ethoxyphenyl)methyl]-3-piperidylamine hydrochloride,
(R)-1-[(3-chloro-4-ethoxyphenyl)methyl]-3-piperidylamine hydrochloride,
1-{[3,5-dichloro-4-(2-methoxyethoxy)phenyl]methyl}-4-piperidylamine hydrochloride,
1-{[3-chloro-4-(2-methoxyethoxy)phenyl]methyl}-4-piperidylamine hydrochloride,
(S)-1-{[3,5-dichloro-4-(cyclopropylmethoxy)phenyl]methyl}-3-piperidylamine hydrochloride,
(R)-1-{[3,5-dichloro-4-(cyclopropylmethoxy)phenyl]methyl}-3-piperidylamine hydrochloride,
1-{[3,5-dichloro-4-(cyclopropylmethoxy)phenyl]methyl}-4-piperidylamine hydrochloride,
(R)-1-{2-[3-chloro-4-(cyclopropylmethoxy)phenyl]ethyl}-3-pyrrolidinylamine hydrochloride,
(S)-1-{2-[3-chloro-4-(cyclopropylmethoxy)phenyl]ethyl}-3-pyrrolidinylamine hydrochloride,
({(S)-1-[(3-chloro-5-ethoxyphenyl)methyl]-3-pyrrolidinyl}methyl)amine hydrochloride,
and ({1-[(5-chloro-2-ethoxyphenyl)methyl]-3-azetidinyl}methyl)amine hydrochloride.

In another particularly preferred embodiment, the compound of formula (I) is a compound selected from the group consisting of
(S)-1-(2-ethoxy-5-(trifluoromethyl)benzyl)-3-methylpiperazine,
(S)-(1-(2-ethoxy-5-(trifluoromethyl) benzyl) pyrrolidin-3-yl) methanamine,
(S)-(1-(5-chloro-2-propoxybenzyl) pyrrolidin-3-yl) methanamine,
(S)-(1-(5-chloro-2-(cyclopropylmethoxy)benzyl)pyrrolidin-3-yl)methanamine,
(S)-(1-(4,5-dichloro-2-ethoxybenzyl)pyrrolidin-3-yl)methanamine,
(S)-(1-(4,5-dichloro-2-(cyclopropylmethoxy)benzyl)pyrrolidin-3-yl)methanamine,
(S)-(1-(5-chloro-2-ethoxybenzyl)piperidin-3-yl)methanamine,
(S)-(1-(5-chloro-2-(cyclopropylmethoxy)benzyl)piperidin-3-yl)methanamine,
(S)-1-(5-chloro-2-ethoxybenzyl)-3-methylpiperazine,
1-(5-chloro-2-ethoxybenzyl)-4-ethylpiperazine,
1-(5-chloro-2-ethoxybenzyl)-N,N-dimethylpiperidin-4-amine,
(S)-(1-(5-chloro-2-ethoxybenzyl)pyrrolidin-3-yl)methanamine,
(S)-(1-(5-chloro-2-ethoxybenzyl)pyrrolidin-2-yl)methanamine,
(R)-(1-(5-chloro-2-ethoxybenzyl)pyrrolidin-2-yl)methanamine,
(R)-1-(5-chloro-2-ethoxybenzyl)piperidin-3-amine,
(S)-1-(5-chloro-2-ethoxybenzyl)piperidin-3-amine,
(1-(5-chloro-2-ethoxybenzyl)azetidin-3-yl)methanamine,
1-(5-chloro-2-ethoxybenzyl)-4-isopropylpiperazine,
(S)-1-(5-chloro-2-ethoxyphenethyl)piperidin-3-amine,
(S)-(1-(3-chloro-4-(cyclopropylmethoxy)phenethyl)pyrrolidin-3-yl)methanamine,
(R)-(1-(2-ethoxy-4,5-difluorobenzyl)pyrrolidin-3-yl)methanamine,
(S)-(1-(2-ethoxy-4,5-difluorobenzyl)pyrrolidin-3-yl)methanamine,
(R)-(1-(3-chloro-4-ethoxybenzyl)pyrrolidin-3-yl) methanamine,
(R)-2-(2-((3-(aminomethyl)pyrrolidin-1-yl)methyl)-4-chlorophenoxy)ethan-1-ol,
(S)-2-(2-((3-(aminomethyl)pyrrolidin-1-yl)methyl)-4-chlorophenoxy)ethan-1-ol,
(R)-(1-(2-butoxy-5-chlorobenzyl)pyrrolidin-3-yl)methanamine,
(S)-(1-(2-butoxy-5-chlorobenzyl)pyrrolidin-3-yl)methanamine,
(S)-(4-(5-chloro-2-ethoxybenzyl)morpholin-2-yl)methanamine,
(R)-(4-(5-chloro-2-ethoxybenzyl)morpholin-2-yl)methanamine,
(S)-(1-(3-chloro-4-ethoxybenzyl)pyrrolidin-3-yl)methanamine,
(S)-(1-(3-chloro-5-ethoxybenzyl)pyrrolidin-3-yl)methanamine,
(S)-3-methyl-1-(2-propoxy-5-(trifluoromethoxy)benzyl) piperazine,
(S)-(1-(2-(6-chlorobenzo[d][1,3]dioxol-4-yl)ethyl)pyrrolidin-3-yl)methanamine,
(S)-(1-(2,3-dichloro-6-ethoxyphenethyl)pyrrolidin-3-yl)methanamine,
(S)-(1-(4,5-dichloro-2-ethoxyphenethyl)pyrrolidin-3-yl)methanamine,
(R)-1-(2-ethoxy-5-(trifluoromethyl)benzyl)-3-methylpiperazine,
(S)-1-(4,5-dichloro-2-ethoxybenzyl)-3-methylpiperazine,
(S)-1-(2-ethoxy-5-(trifluoromethoxy)benzyl)-3-methylpiperazine,
(1-(3-chloro-4-ethoxybenzyl)pyrrolidin-3-yl)methanamine,
1-(5-chloro-2-ethoxybenzyl)piperazine,
(4-(5-chloro-2-ethoxybenzyl)morpholin-2-yl)methanamine,
(S)-(1-(2-ethoxy-5-(trifluoromethyl)phenethyl)pyrrolidin-3-yl)methanamine,
(S)-(1-(5-chloro-2-propoxyphenethyl)piperidin-3-yl)methanamine,
(S)-(1-(5-chloro-2-(cyclopropylmethoxy)phenethyl)pyrrolidin-3-yl)methanamine,
(S)-(1-(5-chloro-2-ethoxyphenethyl)piperidin-3-yl)methanamine,
(S)-1-(5-chloro-2-(cyclopropylmethoxy)benzyl)-3-methylpiperazine,
(S)-1-(5-chloro-2-propoxybenzyl)-3-methylpiperazine,
(S)-(1-(5-chloro-2-propoxyphenethyl)pyrrolidin-3-yl)methanamine,
(S)-(1-(5-chloro-2-ethoxyphenethyl)pyrrolidin-3-yl)methanamine,
(S)-1-(1-(5-chloro-2-ethoxybenzyl)pyrrolidin-3-yl)-N,N-dimethyl methanamine,
(S)-1-(1-(5-chloro-2-ethoxybenzyl)pyrrolidin-3-yl)-N-methylmethanamine,
(S)-(1-(5-chloro-2-isobutoxybenzyl)pyrrolidin-3-yl)methanamine,
(S)-(1-(5-chloro-2-isopropoxybenzyl)pyrrolidin-3-yl)methanamine,
(S)-(1-(5-chloro-2-(2-methoxyethoxy)benzyl)pyrrolidin-3-yl)methanamine,
(S)-(1-((6-chlorobenzo[d][1,3]dioxol-4-yl)methyl)pyrrolidin-3-yl)methanamine,
(R)-(1-(5-chloro-2-ethoxybenzyl)pyrrolidin-3-yl)methanamine,
(S)-(1-(3-chloro-4-ethoxy-2-fluorobenzyl)pyrrolidin-3-yl)methanamine,
(S)-(1-(5-chloro-4-ethoxy-2-fluorobenzyl)pyrrolidin-3-yl)methanamine,
(R)-4-((3-(aminomethyl)pyrrolidin-1-yl)methyl)-2-chloro-5-ethoxy benzonitrile,
(S)-4-((3-(aminomethyl)pyrrolidin-1-yl)methyl)-2-chloro-5-ethoxybenzonitrile,
(S)-(4-(5-chloro-2-ethoxyphenethyl)morpholin-2-yl)methanamine,
(S)-3-(2-(3-(aminomethyl)piperidin-1-yl)ethyl)-4-ethoxybenzonitrile,
(S)-3-(2-(3-(aminomethyl)pyrrolidin-1-yl)ethyl)-4-(cyclopropylmethoxy)benzonitrile,
(S)-(1-(3-chloro-4-ethoxyphenethyl)piperidin-3-yl)methanamine,
(S)-1-(5-chloro-2-ethoxybenzyl)pyrrolidin-3-amine,
(S)-(1-(3,5-dichloro-4-ethoxybenzyl)pyrrolidin-3-yl)methanamine,
1-[2-(3-chloro-4-ethoxyphenyl)ethyl]-4-piperidylamine,
(S)-1-{2-[3,5-dichloro-4-(2-methoxyethoxy)phenyl]ethyl}-3-piperidylamine,
(R)-1-{[3,5-dichloro-4-(cyclopropylmethoxy)phenyl]methyl}-3-pyrrolidinylamine,
(S)-1-{2-[3,5-dichloro-4-(cyclopropylmethoxy)phenyl]ethyl}-3-piperidylamine,
1-[(3-chloro-4-ethoxyphenyl)methyl]-4-piperidylamine,
(R)-1-{2-[3,5-dichloro-4-(2-methoxyethoxy)phenyl]ethyl}-3-piperidylamine,
(S)-1-{[3,5-dichloro-4-(cyclopropylmethoxy)phenyl]methyl}-3-pyrrolidinylamine,
(R)-1-{2-[3,5-dichloro-4-(cyclopropylmethoxy)phenyl]ethyl}-3-piperidylamine,
(S)-1-{2-[3-chloro-4-(cyclopropylmethoxy)phenyl]ethyl}-3-piperidylamine,
(R)-1-{2-[3-chloro-4-(cyclopropylmethoxy)phenyl]ethyl}-3-piperidylamine,
1-{[3-chloro-4-(cyclopropylmethoxy)phenyl]methyl}-4-piperidylamine,
5-{[(R)-3-(Aminomethyl)-1-pyrrolidinyl]methyl}-3-chloro-2-ethoxybenzonitrile,
3-{2-[(S)-3-Amino-1-piperidyl]ethyl}-4-ethoxybenzonitrile,
3-{2-[(R)-3-Amino-1-piperidyl]ethyl}-4-ethoxybenzonitrile,
5-{2-[(S)-3-amino-1-pyrrolidinyl]ethyl}-2-ethoxybenzonitrile,
3-{2-[(S)-3-Amino-1-pyrrolidinyl]ethyl}-4-ethoxybenzonitrile,
3-{2-[(R)-3-amino-1-pyrrolidinyl]ethyl}-4-ethoxybenzonitrile,
(R)-1-[2-(4-ethoxy-3-fluorophenyl)ethyl]-3-pyrrolidinylamine,
(R)-1-{2-[3-chloro-4-(2-methoxyethoxy)phenyl]ethyl}-3-piperidylamine,
1-[(3,5-Dichloro-4-ethoxyphenyl)methyl]-4-piperidylamine,
1-{2-[3,5-Dichloro-4-(cyclopropylmethoxy)phenyl]ethyl}-4-piperidylamine,
(S)-1-[(3-chloro-4-ethoxyphenyl)methyl]-3-piperidylamine,
(R)-1-[(3-chloro-4-ethoxyphenyl)methyl]-3-piperidylamine,
1-{[3,5-dichloro-4-(2-methoxyethoxy)phenyl]methyl}-4-piperidylamine,
1-{[3-chloro-4-(2-methoxyethoxy)phenyl]methyl}-4-piperidylamine,
(S)-1-{[3,5-dichloro-4-(cyclopropylmethoxy)phenyl]methyl}-3-piperidylamine,
(R)-1-{[3,5-dichloro-4-(cyclopropylmethoxy)phenyl]methyl}-3-piperidylamine,
1-{[3,5-dichloro-4-(cyclopropylmethoxy)phenyl]methyl}-4-piperidylamine,
(R)-1-{2-[3-chloro-4-(cyclopropylmethoxy)phenyl]ethyl}-3-pyrrolidinylamine,
(S)-1-{2-[3-chloro-4-(cyclopropylmethoxy)phenyl]ethyl}-3-pyrrolidinylamine,
({(S)-1-[(3-chloro-5-ethoxyphenyl)methyl]-3-pyrrolidinyl}methyl)amine, and
({1-[(5-chloro-2-ethoxyphenyl)methyl]-3-azetidinyl}methyl)amine.

The compound according to formula (I) may be administered via any suitable route of administration including oral, subcutaneous, intravenous, intravenous or epidural patient controlled analgesia (PCA and PCEA), intramuscular, intrathecal, epidural, intracistemal, intraperitoneal, transdermal, topical, buccal, sublingual, transmucosal, inhalation, intra-articular, intranasal, rectal or ocular administration.

The compound according to formula (I) may be administered in any suitable dosing scheme ranging from hourly, daily, weekly, monthly to yearly administration. The dosing scheme may also include cyclic dosing schemes involving time periods without administration after which administration is resumed. The dosing scheme may be adapted for the route of administration applied. The compound according to formula (I) may be administered in a fixed amount or may be administered in an amount that is adapted to the patient's weight.

The compound according to formula (I) may also be administered in combination with one or more further therapeutic agents. The compound according to formula (I) and the one or more further therapeutic agents may be provided in a combined formulation or in separate formulations. Administration may occur in parallel (at the same time point) or sequentially (at different time points). In one embodiment, compound according to formula (I) and the one or more further therapeutic agents are each administered according to their established dosing regimens.

The present invention also provides a method of treating a patient, wherein the method comprises administering a therapeutically effective amount of the compound according to formula (I) to a patient in need thereof. In one embodiment, the patient suffers from any of the diseases or conditions listed herein.

The present invention also provides a use of the compound according to formula (I) in a cosmetic method. The method may comprise the step of applying the compound according to formula (I) to the skin of a mammal. In one embodiment, the use is for reducing cutaneous aging.

It is yet a further object of the present invention to provide a use of the compound according to formula (I) for stimulating autophagy in an in-vitro assay. The assay may be a cell-based assay involving a fluorescent dye. Stimulation of autophagy may be analyzed by measuring increased acidic vesicle formation. In one embodiment, acidic vesical formation is increased by the compound according to formula (I) by at least 25%, by at least 50% or by at least 100% in comparison to untreated control cells. In one embodiment, the cell-based assay is a mammalian-cell based assay.

The present invention is further illustrated by the following examples:

### EXAMPLES

### Materials and Methods, Synthetic Procedures

The compounds of the present invention were prepared according to the following synthetic procedures and Examples and are further exemplified by the following specific examples. Unless otherwise indicated in the following, the starting materials are obtained from commercial suppliers, which is indicated for example by the CAS numbers, and used without further purification. However, it is to be understood that in case the CAS number is not indicated below a starting material, this does not mean that the starting material was not obtained from commercial suppliers. Further, it is to be understood that the starting materials for the examples are either commercially available or are readily prepared by standard methods from known materials.

### Liquid Chromatography Mass Spectrometry (LC-MS)

For compounds 5 and 6 LC-MS spectra were recorded on a Waters 996 Photodiode Array Detector equipped with Waters Micromass ZQ detector.

Standard set-up:
Column ID WELCH C18 4.6×150 mm, 5 ␣m
Machine Details Column temperature: 35° C., Auto sampler temperature: 15° C., Mobile Phase A: 5 mM Ammonium Acetate and 0.1% Formic acid (pH=3.50) in Milli Q water, Mobile Phase B: Methanol
Mobile phase gradient details T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate: 1.0 mL/min, Run Time: 17 min, UV Detection Method: PDA.
Mass parameter Probe: ESI, Mode of Ionisation: Positive and Negative, Cone voltage: 30 and 10 V, capillary voltage: 3.0 KV, Extractor Voltage: 2 V, Rf Lens: 0.1 V, Temperature of source: 120° C., Temperature of Probe: 400° C., Cone Gas Flow: 100 L/Hr, Desolvation Gas flow: 800 L/Hr.

For all other compounds, LC-MS spectra were recorded on a Waters Acquity Ultra performance LC system equipped with a Photodiode Array (PDA) detector with an attached Quadrupole Dalton (QDa) detector.

Standard set-up:
Column ID: X-BRIDGE BEH C18 2.1×50 mm, 2.5 μm
Machine details Column temperature: 35° C., Auto sampler temperature: 5° C., Mobile Phase A: 0.1% Formic acid in Milli Q water (pH=2.70), Mobile Phase B: 0.1% Formic acid in Milli Q water:Acetonitrile (10:90).
Mobile phase gradient details T=0 min (97% A, 3% B) flow: 0.8 mL/min; T=0.75 min (97% A, 3% B) flow: 0.8 mL/min; gradient to T=2.7 min (2% A, 98% B) flow: 0.8 mL/min; gradient to T=3 min (0% A, 100% B) flow: 1 mL/min; T=3.5 min (0% A, 100% B) flow: 1 mL/min; gradient to T=3.51 min (97% A, 3% B) flow: 0.8 mL/min; end of run at T=4 min (97% A, 3% B), Flow rate: 0.8 mL/min, Run Time: 4 min, UV Detection Method: PDA.
Mass parameter Probe: ESI, Mode of Ionisation: Positive and Negative, Cone voltage: 30 V and 10 V, capillary voltage: 0.8 KV, Extractor Voltage: 1 V, Rf Lens: 0.1 V, Temperature of source: 120° C., Temperature of Probe: 600° C. Cone Gas Flow: Default, Desolvation Gas flow: Default.

### High-Performance Liquid Chromatography (HPLC)

For Compound 64 HPLC was carried out using a Waters Alliance e2695 equipped with a 2998 Photodiode Array (PDA) detector.

Set-up used:
Column temperature 25° C.
Auto sampler temperature 25° C.
Mobile Phase A 0.05% ammonium hydroxide solution in HPLC water
Mobile Phase B 100% ACETONITRILE
Mobile phase gradient details T=0 min (10% A, 90% B) flow: 1 mL/min; T=7 min (90% A, 10% B) flow 1 mL/min; gradient to T=9 min (100% A, 0% B) flow: 1 mL/min; gradient to T=14 min (100% A, 0% B) flow: 1 mL/min; T=14.01 min (10% A, 90% B) flow: 1 mL/min; gradient to T=17 min (10% A, 90% B) flow: 1 mL/min; end of run at T=17 min (10% A, 90% B), Flow rate: 1 mL/min, Run Time: 17 min, UV Detection Method: PDA.

For all other compounds, HPLC was carried out using a SHIMADZU i-Series LC-2050C 3D system with a Photodiode Array (PDA) detector with one of the following columns: Sunfire C18 (150×4.6 mm), 3.5 μm; Xtimate C18 (150×4.6 mm), 5 μm; Atlantis C18 (250×4.6 mm), 5 μm; Atlantis C18 (150×4.6 mm), 5 μm.

Standard set-up for SHIMADZU i-Series LC-2050C 3D with PDA detector:
Column temperature 25° C.
Auto sampler temperature 25° C.
Mobile Phase A 0.05% Trifluoroacetic acid in HPLC water
Mobile Phase B 100% Acetonitrile
Mobile phase gradient details T=0 min (90% A, 10% B) flow: 1 mL/min; T=7 min (10% A, 90% B) flow: 1 mL/min; gradient to T=9 min (0% A, 100% B) flow: 1 mL/min; gradient to T=14 min (0% A, 100% B) flow: 1 mL/min; T=14.01 min (90% A, 10% B) flow: 1 mL/min; gradient to T=17 min (90% A, 10% B) flow: 1 mL/min; end of run at T=17 min (90% A, 10% B), Flow rate: 1 mL/min, Run Time: 17 min, UV Detection Method:—PDA.

### Chiral High-Performance Liquid Chromatography (Chiral HPLC)

Chiral HPLC was carried out using a Waters SFC Investigator system with 2998 Photodiode Array (PDA) detector or a Shimadzu LC-20 AD system with Diode-Array Detection (DAD) detector with one of the following columns: CHIRALPAK® IA (250×4.6 mm), 5 ␣m; CHIRALPAK® IG (250×4.6 mm), 5 ␣m; CHIRALCEL® OD-H (250×4.6 mm), 5 ␣m; CHIRALPAK® IH (250×4.6 mm), 5 ␣m; YMC CELLULOSE SC (250×4.6 mm), 5 ␣m; CHIRALPAK® IB-N (250×4.6 mm), 5 ␣m; CHIRALPAK® AD-H (250*4.6 mm), 5 ␣m.

Full details on chiral HPLC for a representative selection of compounds are provided further below in the detailed synthetic procedures.

### Experimental Protocols for Intermediates which were not Readily Available

### Synthesis of 3-chloro-4-(cyclopropylmethoxy)benzaldehyde

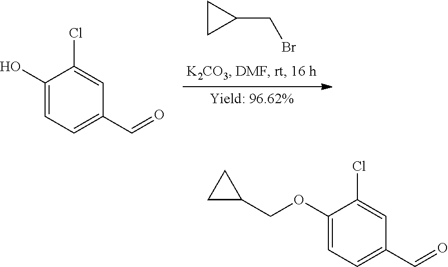 [SMILES: O=Cc1ccc(O)c(Cl)c1 ; BrCC1CC1 ; O=Cc1ccc(OCC2CC2)c(Cl)c1]

To a stirred solution of 3-chloro-4-hydroxybenzaldehyde (5.0 g, 31.9 mmol) in DMF (50 mL) at 0° C., K₂CO₃ (13.20 g, 95.7 mmol) was added. After 30 min, (bromomethyl)cyclopropane (6.46 g, 47.9 mmol) was added at 0° C. and the reaction mixture stirred at room temperature for 16 h. The progress of the reaction was monitored by TLC analysis. After completion of the reaction, the reaction mixture was poured into ice-cold water (300 mL) and extracted with EtOAc (2×200 mL). The combined organic fractions were dried over sodium sulfate and concentrated under reduced pressure to provide 3-chloro-4-(cyclopropylmethoxy)benzaldehyde (6.5 g, 96.62%) as an off-white solid.

LCMS [ESI, M+1]: 210.9 (RT: 1.978 min, Purity: 93.95%),

Alternatively, 3-chloro-4-(cyclopropylmethoxy)benzaldehyde can be synthesized as follows.

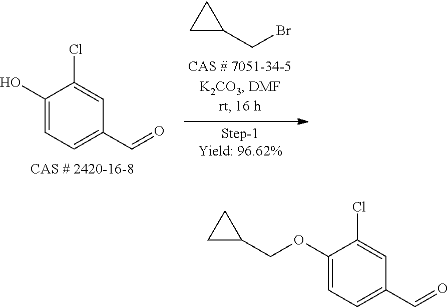 [SMILES: BrCC1CC1 ; O=Cc1ccc(O)c(Cl)c1 ; O=Cc1ccc(OCC2CC2)c(Cl)c1]

To a stirred solution of 3-chloro-4-hydroxybenzaldehyde (5.0 g, 31.9 mmol) in DMF (50 mL) at 0° C., K₂CO₃ (13.20 g, 95.7 mmol) was added. After 30 min, (bromomethyl)cyclopropane (6.46 g, 47.9 mmol) was added at 0° C. and the resulting reaction mixture stirred at room temperature for 16 h. The progress of the reaction was monitored by TLC analysis. After completion of the reaction, the reaction mixture was poured into ice-cold water (300 mL) and extracted with EtOAc (2×200 mL). The combined organic fractions were dried over Na₂SO₄ and concentrated under reduced pressure to provide 3-chloro-4-(cyclopropylmethoxy)benzaldehyde (6.5 g, 96.62%) as an off-white solid.

LCMS [ESI, M+1]: 210.9 (RT: 1.978 min, Purity: 93.95%).

### Synthesis of 2-(3-chloro-4-(cyclopropylmethoxy)phenyl)acetaldehyde

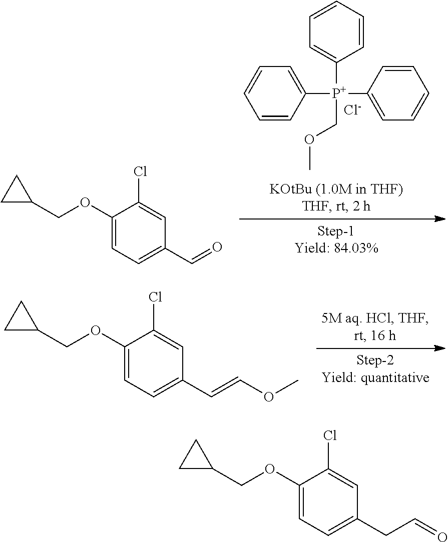

### Step-1: Synthesis of 4-[(E)-2-Methoxyethenyl]-2-chloro-1-(cyclopropylmethoxy)benzene

To a stirred solution of (Methoxymethyl)triphenylphosphonium chloride (3.25 g, 9.49 mmol) in THF (40 mL) at 0° C., 1M KOtBu in THF (23.7 mL, 23.7 mmol) was added. After 1 h, 3-chloro-4-(cyclopropylmethoxy)benzaldehyde (1.0 g, 4.74 mmol) was added at 0° C. and the resulting reaction mixture stirred at 0° C. for 15 min. The progress of the reaction was monitored by TLC analysis. After completion of the reaction, the reaction mixture was diluted with EtOAc (70 mL) and filter through Celite® The filtrate was dried over sodium sulfate and concentrated under reduced pressure. The crude material was purified by normal phase column chromatography (100% Hexane) to provide 4-[(E)-2-Methoxyethenyl]-2-chloro-1-(cyclopropylmethoxy)benzene (1.0 g, 84.03%) as a yellow oil.

### Step-2: Synthesis of 2-(3-chloro-4-(cyclopropylmethoxy)phenyl)acetaldehyde

To a stirred solution of 4-[(E)-2-Methoxyethenyl]-2-chloro-1-(cyclopropylmethoxy)benzene (1.0 g, 3.95 mmol) in THF (10.0 mL, 10.0V) at room temperature, 5M aq. HCl (5.0 mL, 5.0V) was added. The reaction mixture was stirred at room temperature for 16 h. The progress of the reaction was monitored by TLC analysis. After completion of the reaction, the reaction mixture was diluted with saturated NaHCO₃ solution (75 mL) and extracted with EtOAc (3×50 mL). The combined organic fractions were dried over sodium sulfate and concentrated under reduced pressure to provide 2-(3-chloro-4-(cyclopropylmethoxy)phenyl) acetaldehyde (0.9 g, Quantitative yield) as a yellow oil. Product formation was confirmed by TLC analysis using the 2,4-DNP stain and used without further purification.

Alternatively, 2-(3-chloro-4-(cyclopropylmethoxy)phenyl)acetaldehyde can be synthesized as follows.

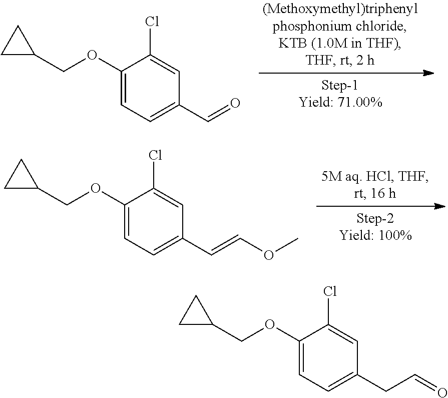

### Step-1: Synthesis of (E)-2-chloro-1-(cyclopropyl methoxy)-4-(3-methoxyallyl) benzene

To a stirred solution of 2-(Methoxymethyl) triphenyl phosphonium chloride (4.88 g, 14.2 mmol) in THF (60 mL) at 0° C., 1M KOtBu (KTB) in THF (35.5 mL, 35.5 mmol) was added. 1H, 3-chloro-4 (cyclopropyl methoxy) benzaldehyde (1.5 g, 0.71 mmol) was then added at 0° C. and the resulting reaction mixture was stirred at 0° C. for 15 min. The progress of the reaction was monitored by TLC analysis. After completion of the reaction, the reaction mixture was diluted with EtOAc (100 mL) and filtered through Celite®. The filtrate was dried over Na₂SO₄ and concentrated under reduced pressure. The crude material was purified by normal phase column chromatography (100% Hexane) to provide (E)-2-chloro-1-(cyclopropyl methoxy)-4-(3-methoxyallyl) benzene (1.2 g, 71.00%) as an off-white liquid.

### Step-2: Synthesis of 2-(3-chloro-4-(cyclopropylmethoxy)phenyl)acetaldehyde

To a stirred solution of (E)-2-chloro-1-(cyclopropyl methoxy)-4-(3-methoxyallyl) benzene (1.2 g, 5.02 mmol) in THF (12 mL) at room temperature, 5M aq. HCl (6.0 mL, 5.0V) was added. The reaction mixture was stirred at room temperature for 16 h. The progress of the reaction was monitored by TLC analysis. After completion of the reaction, the reaction mixture was poured into a sat. NaHCO₃ solution (50 mL) and extracted with EtOAc (3×70 mL). The combined organic fractions were dried over Na₂SO₄ and concentrated under reduced pressure to provide 2-(3-chloro-4-(cyclopropyl methoxy) phenyl) acetaldehyde (1.2 g, Quantitative yield) as a yellow oil.

### Synthesis of 3,5-dichloro-4-(2-methoxyethoxy)benzaldehyde

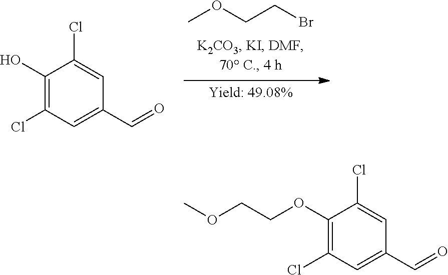

To a stirred solution of 3,5-dichloro-4-hydroxybenzaldehyde (5.0 g, 26.17 mmol) in DMF (50 mL, 10V) at room temperature, K₂CO₃ (7.26 g, 52.35 mmol) was added. The reaction mixture was stirred at room temperature for 30 min. KI and 1-bromo-2-methoxyethane (7.28 g, 52.35 mmol) were then added at room temperature. The reaction mixture was stirred 70° C. for 4 h. The progress of the reaction was monitored by TLC analysis. After completion of the reaction, the reaction mixture was poured into water (100 mL) and extracted with EtOAc (3×100 mL). The combined organic fractions were dried over sodium sulfate and concentrated under reduced pressure to provide 3,5-dichloro-4-(2-methoxyethoxy) benzaldehyde (3.2 g, 49.08%) as a brown sticky solid.

¹H NMR (400 MHz, d₆-DMSO): δ 9.90 (s, 1H), 8.00 (s, 2H), 4.27-4.23 (m, 2H), 3.72-3.68 (m, 2H), 3.30 (s, 3H).

### Synthesis of 3,5-dichloro-4-(cyclopropylmethoxy) benzaldehyde

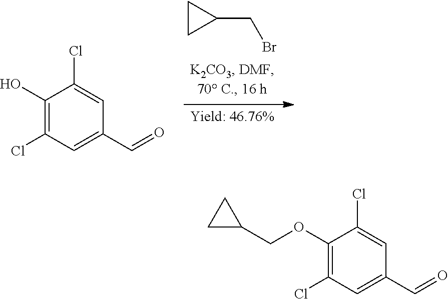 [SMILES: BrCC1CC1 ; O=Cc1cc(Cl)c(O)c(Cl)c1 ; O=Cc1cc(Cl)c(OCC2CC2)c(Cl)c1]

To a stirred solution of 2,3-dichloro-4-hydroxybenzaldehyde (0.5 g, 2.61 mmol) in DMF (5 mL) at room temperature, K₂CO₃ (1.08 g, 7.8 mmol) was added. After 10 min (bromomethyl)cyclopropane (0.70, 5.2 mmol) and potassium iodide (0.43 g, 2.61 mmol) were added at room temperature. The reaction mixture was stirred at 70° C. for 16 h. The progress of the reaction was monitored by TLC analysis. After completion of the reaction, the reaction mixture was poured into water (100 mL) and extracted with EtOAc (100 mL×2). The combined organic fractions were dried over sodium sulfate and concentrated under reduced pressure to provide 3,5-dichloro-4-(cyclopropylmethoxy) benzaldehyde (0.3 g, 31.17% yield) as a yellow sticky solid.

¹H NMR (400 MHz, CD₃OD): δ 9.87 (s, 1H), 7.94 (s, 2H), 4.02 (d, J=7.2 Hz, 2H), 1.40-1.30 (m, 1H) 0.65-0.59 (m, 2H), 0.38-0.34 (m, 2H).

Alternatively, 3,5-dichloro-4-(cyclopropyl methoxy) benzaldehyde can be synthesized as follows.

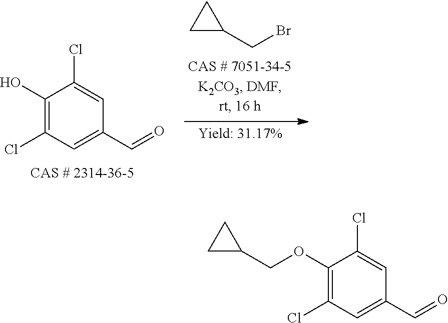 [SMILES: BrCC1CC1 ; O=Cc1cc(Cl)c(O)c(Cl)c1 ; O=Cc1cc(Cl)c(OCC2CC2)c(Cl)c1]

To a stirred solution of 2,3-dichloro-4-hydroxybenzaldehyde (0.5 g, 2.61 mmol) in DMF (5 mL) at room temperature, K₂CO₃ (1.08 g, 7.84 mmol) was added. After 15 min, (bromomethyl) cyclopropane (0.42, 3.13 mmol) was added at room temperature. Then the reaction mixture was stirred at room temperature for 16 h. The progress of the reaction was monitored by TLC analysis. After completion of the reaction, the reaction mixture was poured into water (100 mL) and extracted with EtOAc (2×100 mL). The combined organic fractions were dried over Na₂SO₄ and concentrated under reduced pressure to provide 3,5-dichloro-4-(cyclopropyl methoxy) benzaldehyde (0.4 g, 31.17% yield) as a yellow sticky solid.

¹H NMR (400 MHz, d₆-DMSO): δ 9.90 (s, 1H), 8.02 (s, 2H), 3.97 (d, J=7.2 Hz, 2H), 1.31-1.23 (m, 1H) 0.60-0.55 (m, 2H), 0.33-0.31 (m, 2H).

### Experimental Protocol for Compound 1

### Synthetic Scheme

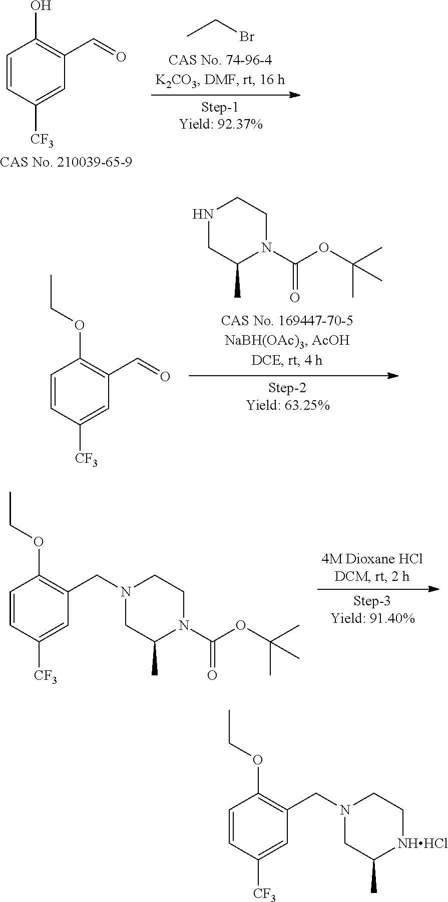

### Step-1: Synthesis of 2-ethoxy-5-(trifluoromethyl)benzaldehyde

### Procedure

To a stirred solution of 2-hydroxy-5-(trifluoromethyl)benzaldehyde (0.5 g, 2.62 mmol) in DMF (5 mL) at room temperature, K₂CO₃ (1.08 g, 7.88 mmol) was added. The reaction mixture was stirred at room temperature for 1 h. Then bromoethane (0.57 g, 5.25 mmol) was added to the reaction mixture at room temperature. The reaction mixture was stirred at room temperature for 16 h. The progress of the reaction was monitored by TLC and LCMS analysis. After completion of the reaction, the reaction mixture was poured into cold water (15 mL) and extracted with ethyl acetate (3×20 mL). The combined organic fractions were washed with ice cold water 3-4 times, dried over anhydrous sodium sulphate and concentrated under reduced pressure. The crude material was purified by flash column chromatography (SiO₂; 0-5% EtOAc/Hexane) to provide 2-ethoxy-5-(trifluoromethyl)benzaldehyde (0.53 g, 92.37% yield) as a colorless liquid.

LCMS [ESI, M+1]: 219.0 (RT: 9.009 min, Purity: 98.74%), ¹H-NMR (400 MHz, d₆-DMSO): δ 10.37 (s, 1H), 8.01-7.99 (dd, J=2.4 Hz, 8.8 Hz, 1H), 7.92 (d, J=2.0 Hz, 1H), 7.44 (d, J=8.8, 1H), 4.30 (q, J=7.0 Hz, 2H), 1.42 (t, J=7.0 Hz, 3H).

### Step-2: Synthesis of tert-butyl (S)-4-(2-ethoxy-5-(trifluoromethyl)benzyl)-2-methylpiperazine-1-carboxylate

### Procedure

To a stirred solution of 2-ethoxy-5-(trifluoromethyl)benzaldehyde (0.35 g, 1.60 mmol) in DCE (7 mL) at room temperature, tert-butyl (S)-2-methylpiperazine-1-carboxylate (0.36 g, 1.76 mmol) and acetic acid (0.02 mL, 0.05V) were added. The reaction mixture was stirred at room temperature for 2 h. Then, sodium triacetoxyborohydride (1.02 g, 4.81 mmol) was added portion-wise to the reaction mixture at 0° C. The reaction mixture was allowed to stir at room temperature for 4 h. The progress of the reaction was monitored by TLC and LCMS analysis. After completion of the reaction, the reaction mixture was diluted with CH₂Cl₂ (50 mL) and washed with sat. NaHCO₃ solution (15 mL) and water (25 mL). The organic fractions were dried over anhydrous sodium sulphate and concentrated under reduced pressure. The crude material was purified by flash column chromatography (SiO₂; 0-50% EtOAc/Hexane) to provide tert-butyl (S)-4-(2-ethoxy-5-(trifluoromethyl)benzyl)-2-methylpiperazine-1-carboxylate (0.35 g, 63.25% yield) as a colorless oil.

LCMS [ESI, M+1]: 403.0 (RT: 1.618 min, Purity: 98.77%), ¹H-NMR (400 MHz, CDCl₃): δ 7.76 (bs, 1H), 7.49 (bs, 1H), 6.91 (d, J=7.6 Hz, 1H), 4.35 (bs, 1H), 4.13-4.08 (m, 2H), 3.86 (d, J=9.2 Hz, 1H), 3.55 (bs, 2H), 3.18 (bs, 1H), 2.81 (bs, 1H), 2.65 (bs, 1H), 2.20 (bs, 2H), 1.58-1.41 (m, 15H).

### Step-3: Synthesis of (S)-1-(2-ethoxy-5-(trifluoromethyl)benzyl)-3-methylpiperazine hydrochloride (Compound 1)

### Procedure

To a stirred solution of tert-butyl (S)-4-(2-ethoxy-5-(trifluoromethyl)benzyl)-2-methylpiperazine-1-carboxylate (0.32 g, 0.79 mmol) in CH₂Cl₂ (3.2 mL) at 0° C., 4.0 M HCl in Dioxane (1.6 mL, 5V) was added. The reaction mixture was then stirred at room temperature for 2 h. The progress of the reaction was monitored by TLC and LCMS analysis. After completion of the reaction, the reaction mixture was concentrated under reduced pressure. The crude material was purified by trituration using diethyl ether to provide (S)-1-(2-ethoxy-5-(trifluoromethyl)benzyl)-3-methylpiperazine hydrochloride (0.206 g, 91.40% yield) as a white solid.

LCMS [ESI, M⁺]: 303.0 (RT: 1.113 min, Purity: 98.21%),

HPLC Purity: RT: 3.892 min, Purity: 97.69%,

Chiral HPLC Purity: RT: 1.83 min, Purity: 100.0%,

Instrument Name: Waters SFC Investigator

Chromatographic separation was conducted with Waters SFC Investigator system with 2998 PDA detector. The column used was CHIRALPAK® IA (250×4.6 mm; 5 ␣m) and the compounds were eluted with, Mobile Phase A: Liq·CO2, Mobile Phase B: 0.1% DIETHYLAMINE in METHANOL with 75-25 composition method; with Flow rate=3 ml/min; Column oven temperature 40° C.; ABPR 130 bar, analysis time 4 min.

¹H-NMR (400 MHz, D₂O): δ 7.76-7.74 (dd, J=1.6 Hz, 8.8 Hz, 1H), 7.69 (d, J=2.0 Hz, 1H), 7.17 (d, J=8.8 Hz, 1H), 4.43 (s, 2H), 4.20-4.14 (q, J=7.0 Hz, 2H), 3.73-3.63 (m, 4H), 3.42-3.32 (m, 2H), 3.16 (t, J=12.8 Hz, 1H), 1.36-1.30 (m, 6H).

### Experimental Protocol for Compound 2

### Synthetic Scheme

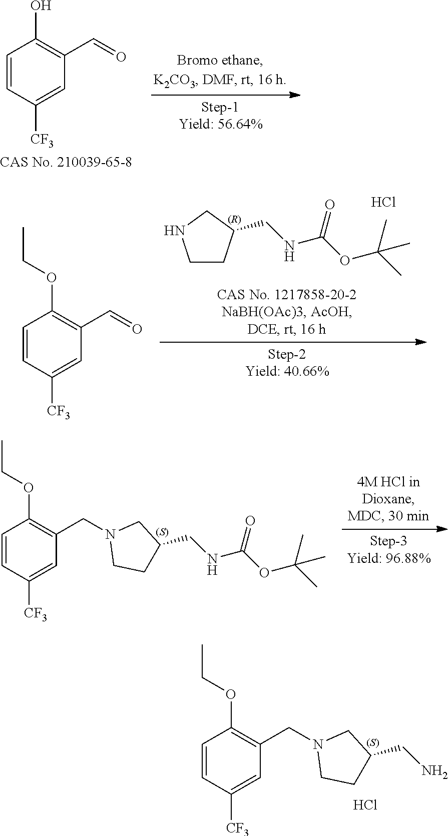

### Step-1: of 2-ethoxy-5-(trifluoromethyl) benzaldehyde

### Procedure

To a stirred solution of 2-hydroxy-5-(trifluoromethyl) benzaldehyde (1.0 g, 5.25 mmol) in DMF (10 mL, 10 V) at room temperature, potassium carbonate (2.17 g, 15.77 mmol) was added. The reaction mixture was stirred at room temperature for 1 h. Then bromo ethane (0.859 g, 7.88 mmol) was added to the reaction mixture. The reaction mixture was stirred at room temperature for 16 h. The progress of the reaction was monitored by TLC and LCMS analysis. After completion of the reaction, the reaction mixture was poured into cold water (100 mL) and extracted with ethyl acetate (2×100 mL). The combined organic fractions were washed with cold water (50 mL), dried over sodium sulphate and concentrated under reduced pressure to provide 2-ethoxy-5-(trifluoromethyl) benzaldehyde (0.650 g, 56.64% yield) as pale yellow solid.

¹H NMR (400 MHz, d₆-DMSO): δ 10.37 (s, 1H), 8.02-7.99 (m, 1H), 7.92 (d, J=4 Hz, 1H), 7.44 (d, J=12 Hz, 1H), 4.33-4.28 (m, 2H), 1.42 (t, J=12 Hz, 3H)

### Step-2: Synthesis of tert-butyl (S)-((1-(2-ethoxy-5-(trifluoromethyl) benzyl) pyrrolidin-3-Yl) methyl) carbamate

### Procedure

To a stirred solution of 2-ethoxy-5-(trifluoromethyl) benzaldehyde (0.2 g, 0.91 mmol) in DCE (2 mL, 10V) at room temperature, tert-butyl (R)-(pyrrolidin-3-ylmethyl) carbamate hydrochloride (0.260 g, 1.10 mmol) and Acetic acid (0.005 mL, 0.05V) were added. The reaction mixture was stirred at room temperature for 2 h. Sodium triacetoxyborohydride (0.582 g, 2.75 mmol) was added portionwise to the reaction mixture at 0° C. After addition, the reaction was stirred at room temperature for 16 h. The progress of the reaction was monitored by TLC and LCMS analysis. After completion of the reaction, the reaction mixture was poured into a saturated solution of sodium bicarbonate (30 mL) and extracted with CH₂Cl₂ (2×30 mL). The combined organic fractions were dried over sodium sulphate and concentrated under reduced pressure. The crude material was purified by flash column chromatography (SiO₂; 22% ethyl acetate in hexane) to provide tert-butyl (S)-((1-(2-ethoxy-5-(trifluoromethyl)benzyl)pyrrolidin-3-yl)methyl)carbamate (0.150 g, 40.66% yield) as a light yellow liquid.

LCMS [ESI, M+1]: 402.96 (RT: 1.542 min, Purity: 99.51%),

### Step-3: Synthesis of (S)-(1-(2-ethoxy-5-(trifluoromethyl)benzyl)pyrrolidin-3-yl)methanamine hydrochloride (Compound 2)

### Procedure

To a stirred solution of tert-butyl (S)-((1-(2-ethoxy-5-(trifluoromethyl) benzyl) pyrrolidin-3-yl) methyl) carbamate (0.150 gm, 0.37 mmol) in CH₂Cl₂ (1.5 mL, 10 v) at 0° C., 4M HCl in Dioxane (0.9 mL, 5V) was added. The reaction mixture was stirred at room temperature for 30 min. The progress of the reaction was monitored by TLC and LCMS analysis. After completion of the reaction, the reaction mixture was concentrated under reduced pressure. The crude material was purified by trituration with diethyl ether to provide (S)-(1-(2-ethoxy-5-(trifluoromethyl)benzyl) pyrrolidin-3-yl)methanamine hydrochloride (0.131 g, 96.88% yield) as a light pink solid.

LCMS [ESI, M+1]: 302.84 (RT: 0.858 min, Purity: 99.72%),

HPLC: RT: 3.912 min, Purity: 100%,

Chiral HPLC: RT: 3.93 min, Purity: 98.02%,

Instrument Name: Waters SFC Investigator

Chromatographic separation was conducted with Waters SFC Investigator system with 2998 PDA detector. The column used was CHIRALPAK® IG (250×4.6 mm; 5 ␣m) and the compounds were eluted with, Mobile Phase A: Liq·CO2, Mobile Phase B: 0.1% METHANOLIC AMMONIA in METHANOL with 75-25 composition method; with Flow rate=3 ml/min; Column oven temperature 40° C.; ABPR 130 bar, analysis time 9 min.

¹H NMR (400 MHz, D₂O): δ 7.74 (d, J=12 Hz, 1H), 7.67 (s, 1H), 7.16 (d, J=8 Hz, 1H), 4.41 (s, 2H), 4.20-4.15 (q, J=14 Hz, 7.2 Hz, 2H), 3.75-3.71 (m, 1H), 3.58-3.55 (m, 1H), 3.35 (m, 1H), 3.07 (m, 3H), 2.65 (m, 1H), 2.37 (m, 1H), 1.94 (s, 1H), 1.35 (t, J=12 Hz, 3H).

### Experimental Protocol for Compound 3

### Synthetic Scheme

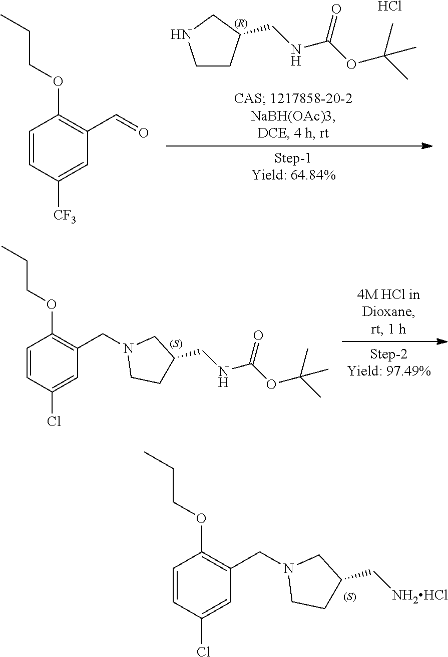

### Step-1: Synthesis of tert-butyl (S)-((1-(5-chloro-2-propoxybenzyl) pyrrolidin-3-yl) methyl) carbamate

### Procedure

To a stirred solution of 5-chloro-2-propoxybenzaldehyde (0.2 g, 1.01 mmol) in DCE (4 mL, 20V) at room temperature, tert-butyl (R)-(pyrrolidin-3-ylmethyl)carbamate hydrochloride (0.28 g, 1.21 mmol) was added. The reaction mixture was stirred at room temperature for 2 h. Then, sodium triacetoxyborohydride (0.64 g, 3.03 mmol) was portion-wise added to the reaction mixture at 0° C. The reaction mixture was allowed to stir at room temperature for 4 h. The progress of the reaction was monitored by TLC and LCMS analysis. After completion of the reaction, the reaction mixture was diluted with CH₂Cl₂ (25 mL) and poured in cold water (30 mL). The organic fraction was washed with saturated sodium bicarbonate solution (25 mL), dried over anhydrous sodium sulphate, and concentrated under reduced pressure. The crude material was purified by flash column chromatography (neutral alumina; 25% ethyl acetate/Hexane) to provide tert-butyl (S)-((1-(5-chloro-2-propoxybenzyl)pyrrolidin-3-yl)methyl)carbamate (0.250 g, 64.84% yield) as a white sticky solid.

LCMS [ESI, M+1]: 383.01 (RT: 1.615 min, Purity: 93.31%)

### Step-2: Synthesis of (S)-(1-(5-chloro-2-propoxybenzyl)pyrrolidin-3-yl)methanamine hydrochloride (Compound 3)

### Procedure

To a stirred solution of tert-butyl (S)-((1-(5-chloro-2-propoxybenzyl)pyrrolidin-3-yl)methyl) carbamate (0.25 g, 0.65 mmol) in CH₂Cl₂ (2.5 mL, 10V) at room temperature, 4.0 M HCl in Dioxane (1.25 mL, 5V) was added. The reaction mixture was stirred at room temperature for 1 h. The progress of the reaction was monitored by TLC and LCMS analysis. After completion of the reaction, the reaction mixture was concentrated under reduced pressure. The crude material was purified by trituration using diethyl ether (10 mL) to provide (S)-(1-(5-chloro-2-propoxybenzyl)pyrrolidin-3-yl) methanamine hydrochloride (0.18 g, 97.49% yield) as a white solid.

LCMS [ESI, M+1]: 282.79 (RT: 0.921 min, Purity: 100%),

HPLC: RT: 3.846 min, Purity: 100%

Chiral HPLC: RT: 2.20 min, Purity: 100%

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% METHANOLIC AMMONIA in METHANOL with 75-25 composition method; with Flow rate=3 ml/min; Column oven temperature 40° C.; ABPR 130 bar, analysis time 5 min.

¹H-NMR (400 MHz, D₂O): δ 7.40 (dd, J=8.9, 2.6 Hz, 1H), 7.36 (d, J=2.6 Hz, 1H), 7.02 (d, J=8.9 Hz, 1H), 4.33 (s, 2H), 4.00 (t, J=6.7 Hz, 2H), 3.62 (s, 1H), 3.43 (s, 2H), 3.06 (qd, J=13.0, 7.4 Hz, 3H), 2.73 (s, 1H), 2.30 (d, J=5.6 Hz, 1H), 1.85-1.67 (m, 4H), 0.92 (t, J=7.4 Hz, 3H).

### Experimental Protocol for Compound 4

### Synthetic Scheme

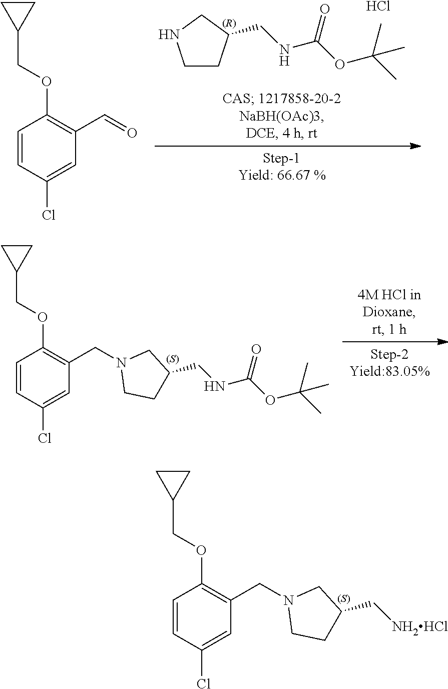

### Step-1: Synthesis of tert-butyl (S)-((1-(5-chloro-2-(cyclopropylmethoxy)benzyl)pyrrolidin-3-yl)methyl)carbamate

### Procedure

To a stirred solution of 5-chloro-2-(cyclopropylmethoxy)benzaldehyde (0.2 g, 0.95 mmol) in DCE (4 mL, 20V) at room temperature, tert-butyl (R)-(pyrrolidin-3-ylmethyl)carbamate hydrochloride (0.26 g, 1.14 mmol) was added. The reaction mixture was stirred at room temperature for 2 h. Sodium triacetoxyborohydride (0.60 g, 2.85 mmol) was added portion-wise to the reaction mixture at 0° C. Then, the reaction mixture was allowed to stir at room temperature for 4 h. The progress of the reaction was monitored by TLC and LCMS analysis. After completion of the reaction, the reaction mixture was diluted with CH₂C₁₂ (25 mL) and poured into cold water (30 mL). The organic fraction was washed with sat. NaHCO₃ solution (25 mL), dried over anhydrous sodium sulphate, and concentrated under reduced pressure. The crude material was purified by flash column chromatography (neutral alumina; 28-30% ethyl acetate/Hexane) to provide tert-butyl (S)-((1-(5-chloro-2-(cyclopropylmethoxy)benzyl)pyrrolidin-3-yl)methyl)carbamate (0.250 g, 66.67% yield) as an offwhite sticky solid.

LCMS [ESI, M+1]: 395.06 (RT: 1.621 min, Purity: 96.87%),

### Step-2: Synthesis of (S)-(1-(5-chloro-2-(cyclopropylmethoxy)benzyl)pyrrolidin-3-yl)methanamine hydrochloride (Compound 4)

### Procedure

To a stirred solution of tert-butyl (S)-((1-(5-chloro-2-(cyclopropylmethoxy)benzyl)pyrrolidin-3-yl)methyl)carbamate (0.25 g, 0.63 mmol) in CH₂Cl₂ (2.5 mL, 10V) at room temperature, 4.0 M HCl in Dioxane (1.25 mL, 5V) was added. The reaction mixture was then stirred at room temperature for 1 h. The progress of the reaction was monitored by TLC and LCMS analysis. After completion of the reaction, the reaction mixture was concentrated under reduced pressure. The crude material was purified by trituration using diethyl ether (10 mL) to provide (S)-(1-(5-chloro-2-(cyclopropylmethoxy)benzyl)pyrrolidin-3-yl)methanamine hydrochloride (0.15 g, 83.05% yield) as an offwhite sticky solid.

LCMS [ESI, M+1]: 296.54 (RT: 0.916 min, Purity: 99.79%),

HPLC: RT: 3.880 min, Purity: 99.21%

Chiral HPLC: RT: 2.09 min, Purity: 99.50%

Instrument Name: Waters SFC Investigator

Chromatographic separation was conducted with Waters SFC Investigator system with 2998 PDA detector. The column used was CHIRALPAK® IA (250×4.6 mm; 5 ␣m) and the compounds were eluted with, Mobile Phase A: Liq·CO2, Mobile Phase B: 0.1% METHANOLIC AMMONIA in METHANOL with 50-50 composition method; with Flow rate=3 ml/min; Column oven temperature 40° C.; ABPR 130 bar, analysis time 7 min.

¹H-NMR (400 MHz, D₂O): δ 7.39 (dd, J=8.8, 2.7 Hz, 1H), 7.36 (d, J=2.5 Hz, 1H), 7.00 (d, J=8.8 Hz, 1H), 4.36 (s, 2H), 3.88 (d, J=7.2 Hz, 2H), 3.64 (s, 1H), 3.45 (s, 2H), 3.07 (ddd, J=21.2, 13.0, 7.4 Hz, 4H), 2.74 (s, 1H), 2.31 (d, J=5.8 Hz, 1H), 1.83 (s, 1H), 1.27-1.17 (m, 1H), 0.59-0.50 (m, 2H), 0.30-0.23 (m, 2H).

### Experimental Protocol for Compound 5

### Synthetic Scheme

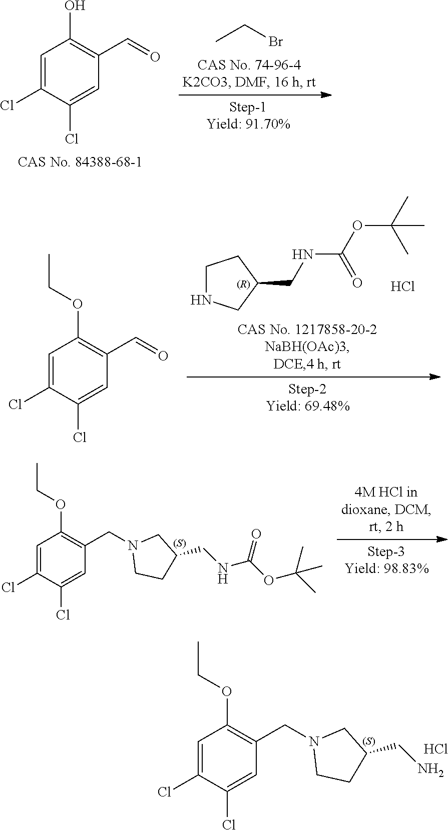

### Step-1: Synthesis of 4,5-dichloro-2-ethoxybenzaldehyde

### Procedure

To a stirred solution of 4,5-dichloro-2-hydroxybenzaldehyde (0.2 g, 1.04 mmol) in DMF (2 mL) at room temperature, Potassium carbonate (0.28 g, 2.09 mmol) was added. The reaction was stirred for 1 h. Then Bromoethane (0.22 g, 2.09 mmol) was added to the reaction mixture. Then the reaction mixture was stirred at room temperature for 16 h. The progress of the reaction was monitored by TLC and LCMS analysis. After completion of the reaction, the reaction mixture was poured into ice-cold water (100 mL) and extracted with ethyl acetate (3×100 mL). The combined organic fractions were dried over sodium sulphate and concentrated under reduced pressure to provide 4,5-dichloro-2-ethoxybenzaldehyde (0.210 g, 91.70% yield) as white solid.

¹H NMR (400 MHz, d₆-DMSO): δ 10.26 (s, 1H), 7.79 (s, 1H), 7.59 (s, 1H), 4.24 (q, J=6.8 Hz, 2H), 1.39 (q, J=6.8 Hz, 3H)

### Step-2: Synthesis of tert-butyl (S)-((1-(4,5-dichloro-2-ethoxybenzyl)pyrrolidin-3-yl)methyl)carbamate

### Procedure

To a stirred solution of 4,5-dichloro-2-ethoxybenzaldehyde (0.18 g, 0.82 mmol) in DCE (3.6 mL) at room temperature, tert-butyl (R)-(pyrrolidin-3-ylmethyl)carbamate hydrochloride (0.19 g, 0.98 mmol) was added. The reaction mixture was stirred at room temperature for 2 h. Sodium triacetoxyborohydride (0.521 g, 2.46 mmol) was added to the reaction mixture in small portions at 0° C. The reaction was then stirred at room temperature for 4 h. The progress of the reaction was monitored by TLC and LCMS analysis. After completion of the reaction, the reaction mixture was poured into a saturated solution of sodium bicarbonate (50 mL) and extracted with DCE (3×100 ml). The combined organic fractions were washed with water (100 mL), dried over sodium sulphate and concentrated under reduced pressure. The crude material was purified by flash column chromatography (SiO₂; 80% EtOAc/Hexane) to provide tert-butyl (S)-((1-(4,5-dichloro-2-ethoxybenzyl)pyrrolidin-3-yl)methyl)carbamate (0.230 g, 69.48% yield) as light pink liquid.

LCMS [ESI, M+1]: 404.60 (RT: 1.683 min, Purity: 99.18%)

Chiral HPLC: RT: 2.00 min, Purity: 100%

¹H NMR (400 MHz, d₆-DMSO): δ 7.45 (s, 1H), 7.21 (s, 1H), 6.86 (t, J=5.2 Hz, 1H), 4.08-4.02 (m, 2H), 3.51 (s, 2H), 2.89 (J=6.4.6 Hz, 2H), 2.42 (s, 1H), 2.28-2.22 (m, 3H), 1.36 (s, 9H), 1.32 (t, J=7.2, 6.8 Hz, 3H), 1.24 (s, 3H)

### Step-3: Synthesis of (S)-(1-(4,5-dichloro-2-ethoxybenzyl)pyrrolidin-3-yl)methanamine hydrochloride (Compound 5)

### Procedure

To a stirred solution tert-butyl (S)-((1-(4,5-dichloro-2-ethoxybenzyl)pyrrolidin-3-yl)methyl) carbamate (0.23 g, 0.57 mmol) in CH₂Cl₂ (2.3 mL, 10V) at 0° C., 4M HCl in Dioxane (1.15 mL, 5V) was added. The reaction mixture was stirred at room temperature for 2 h. The progress of the reaction was monitored by TLC and LCMS analysis. After completion of the reaction, the reaction mixture was concentrated under reduced pressure and the crude material was triturated with diethyl ether to provide (S)-(1-(4,5-dichloro-2-ethoxybenzyl)pyrrolidin-3-yl)methanamine hydrochloride (0.17 g, 98.83% yield) as an off-white solid.

LCMS [ESI, M+1]: 303.07 (RT: 5.324 min, Purity: 98.54%),

HPLC: RT: 3.886 min, Purity: 100%

Chiral HPLC: RT: 2.49 min, Purity: 99.57%

Instrument Name: Waters SFC Investigator

Chromatographic separation was conducted with Waters SFC Investigator system with 2998 PDA detector. The column used was CHIRALCEL OD-H (250×4.6 mm; 5 ␣m) and the compounds were eluted with, Mobile Phase A: Liq·CO2, Mobile Phase B: 0.1% METHANOLIC AMMONIA in METHANOL with 75-25 composition method; with Flow rate=3 ml/min; Column oven temperature 40° C.; ABPR 130 bar, analysis time 5.5 min.

¹H NMR (400 MHz, D₂O): δ 7.48 (s, 1H), 7.24 (s, 1H), 4.35-4.27 (m, 2H), 4.09 (q, J=7.0 Hz, 2H), 3.61 (m, 1H), 3.42 (s, 2H), 3.14-2.96 (m, 3H), 2.73 (m, 1H), 2.29 (m, 1H), 1.82 (m, 1H), 1.32 (t, J=7.0 Hz, 3H).

### Experimental Protocol for Compound 6

### Synthetic Scheme

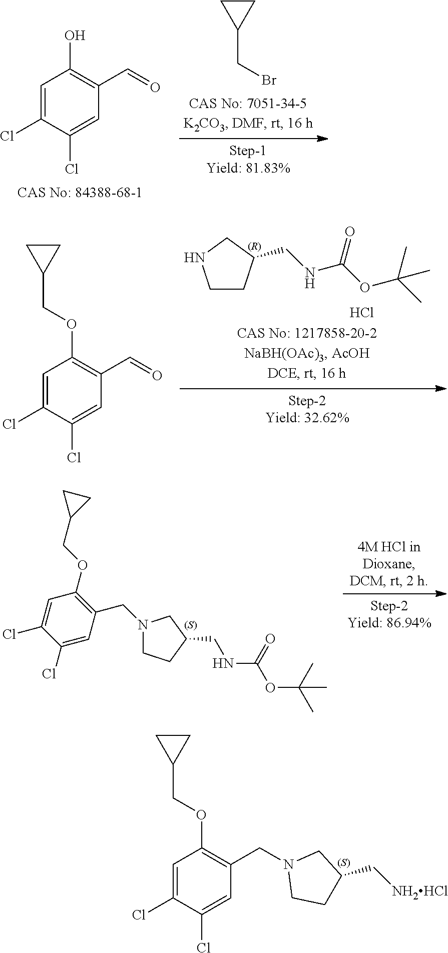

### Step-1: 4,5-dichloro-2-(cyclopropylmethoxy)benzaldehyde

### Procedure

To a stirred solution of 4,5-dichloro-2-hydroxybenzaldehyde (0.200 g, 1.047 mmol) in DMF (2 mL) at room temperature, Potassium carbonate (0.434 g, 3.14 mmol) was added. The reaction mixture was stirred at rt for 1 h. Then (bromomethyl)cyclopropane (0.212 g, 1.570 mmol) was added to the reaction mixture. The reaction mixture was stirred at room temperature for 16 h. The progress of the reaction was monitored by TLC and LCMS analysis. After completion of the reaction, the reaction mixture was poured into ice-cold water (20 mL) and extracted with ethyl acetate (3×20 mL). The combined organic fractions were dried over sodium sulphate and concentrated under reduced pressure. The crude material was purified by flash column chromatography (SiO₂; 21% ethyl acetate in hexane) to provide 4,5-dichloro-2-(cyclopropyl methoxy)benzaldehyde (0.210 g, 81.83% yield) as off white sticky liquid.

¹H NMR (400 MHz, CDCl3): δ 10.41 (s, 1H), 7.94 (s, 1H), 7.07 (s 1H), 7.19 (d, J=8.0 Hz, 2H), 1.31 (t, J=6.9 Hz, 1H), 1.27-1.24 (m, 2H) 0.72-0.79 (m, 2H)

### Step-2: tert-butyl (S)-((1-(4,5-dichloro-2-(cyclopropylmethoxy)benzyl)pyrrolidin-3-yl)methyl) carba mate

### Procedure

To a stirred solution of 4,5-dichloro-2-(cyclopropylmethoxy)benzaldehyde (0.210 g, 0.857 mmol) in DCE (2.1 mL) at room temperature, tert-butyl (R)-(pyrrolidin-3-ylmethyl)carbamate hydrochloride (0.242 g, 1.028 mmol) and Acetic Acid (0.01 mL, 0.05V) were added. The reaction mixture was stirred at room temperature for 3 h. Sodium triacetoxyborohydride (0.542 g, 2.57 mmol) was added to the reaction mixture in small portions at 0° C. The reaction was stirred at room temperature for 16 h. The progress of the reaction was monitored by TLC and LCMS analysis. After completion of the reaction, the reaction mixture was poured into a saturated solution of sodium bicarbonate (20 mL) and extracted with CH₂Cl₂ (3×20 mL). The combined organic fractions were washed with water (20 mL), dried over sodium sulphate and concentrated under reduced pressure. The crude material was purified by flash column chromatography (SiO₂; 60-65% ethyl acetate in hexane) to provide tert-butyl (S)-((1-(4,5-dichloro-2-(cyclopropylmethoxy)benzyl)pyrrolidin-3-yl)methyl)carbamate (0.110 g, 32.62% yield) as a colorless liquid.

LCMS [ESI, M+1]: 430.8 (RT: 1.678 min, Purity: 85.40%)

### Step3: (S)-(1-(4,5-dichloro-2-(cyclopropylmethoxy)benzyl)pyrrolidin-3-yl)methanamine hydrochloride (Compound 6)

### Procedure

To a stirred solution of tert-butyl (S)-((1-(4,5-dichloro-2-(cyclopropylmethoxy)benzyl)pyrrolidin-3-yl) methyl)carbamate (0.110 g, 0.256 mmol) in CH₂Cl₂ (1.1 mL, 10V) at 0° C., 4M HCl in Dioxane (0.2 mL, 0.5V) was added. The reaction mixture was stirred at room temperature for 2 h. The progress of the reaction was monitored by TLC and LCMS analysis. After completion of the reaction, the reaction mixture was concentrated under reduced pressure and the crude material was triturated with diethyl ether (15 mL) to provide (S)-(1-(4,5-dichloro-2-(cyclopropylmethoxy)benzyl)pyrrolidin-3-yl)methanamine hydrochloride (0.080 g, 86.94%) as an off-white solid.

LCMS [ESI, M+1]: 329.2 (RT: 5.799 min, Purity: 96.71%),

HPLC: RT: 4.747 min, Purity: 94.47%

¹H NMR (400 MHz, D₂O): δ 7.48 (s, 1H), 7.22 (s, 1H), 4.65 (s, 2H), 4.34 (s, 2H), 3.87 (d, J=7.2 Hz, 1H), 3.64 (d, J=14.1 Hz, 2H), 3.43 (m, 3H), 2.73 (m, 1H), 2.30 (m, 1H), 1.82 (m, 1H), 1.29-1.13 (m, 1H), 0.58-0.48 (m, 2H), 0.31-0.22 (m, 2H).

### Experimental Protocol for Compound 7

### Synthetic Scheme

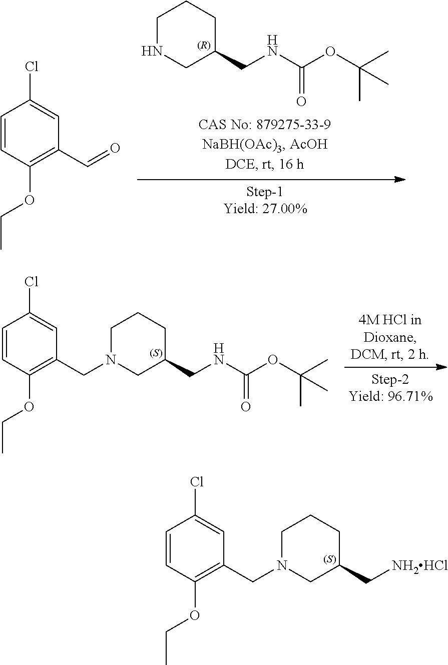

### Step-1: Synthesis of tert-butyl (S)-((1-(5-chloro-2-ethoxybenzyl)piperidin-3-yl)methyl)carbamate

### Procedure

To a stirred solution of 5-chloro-2-ethoxybenzaldehyde (0.250 g, 1.35 mmol) in DCE (2.5 mL) at room temperature, tert-butyl (R)-(piperidin-3-ylmethyl)carbamate (0.347 g, 1.62 mmol) and acetic acid (0.012 mL, 0.05V) were added. The reaction mixture was stirred at room temperature for 3 h. Sodium triacetoxyborohydride (0.857 g, 4.06 mmol) was added portion-wise to the reaction mixture at 0° C. Then the reaction was stirred at room temperature for 16 h. The progress of the reaction was monitored by TLC and LCMS analysis. After completion of reaction, the reaction mixture was poured into a saturated solution of sodium bicarbonate (6 mL) and extracted with CH₂Cl₂ (3×10 mL). The combined organic fractions were washed with water (2×10 mL), dried over sodium sulphate and concentrated under reduce pressure. The crude material was purified by flash column chromatography (SiO₂; 65-70% ethyl acetate in hexane) to provide tert-butyl (S)-((1-(5-chloro-2-ethoxybenzyl)piperidin-3-yl)methyl)carbamate (0.140 g, 27.00% yield) as a light yellow liquid.

LCMS [ESI, M+1]:382.8 (RT: 1.515 min, Purity: 97.89%),

### Step-2: Synthesis of (S)-(1-(5-chloro-2-ethoxybenzyl)piperidin-3-yl)methanamine hydrochloride (Compound 7)

### Procedure

To a stirred solution of tert-butyl (S)-((1-(5-chloro-2-ethoxybenzyl)piperidin-3-yl)methyl)carbamate (0.140 g, 0.36 mmol) in CH₂Cl₂ (1.4 mL, 10V) at 0° C., 4M HCl in Dioxane (0.7 mL, 0.5V) was added. The reaction mixture was stirred at room temperature for 2 h. The progress of the reaction was monitored by TLC and LCMS analysis. After completion of reaction, the reaction mixture was concentrated under reduced pressure to provide crude material which was purified by trituration with diethyl ether to obtain (S)-(1-(5-chloro-2-ethoxybenzyl)piperidin-3-yl)methanamine hydrochloride (0.100 g, 70.58% yield) as an off-white solid.

LCMS [ESI, M+1]: 282.7 (RT: 0.794 min, Purity: 100%),

HPLC: RT: 3.726 min, Purity: 100%,

Chiral HPLC: RT: 2.15, Purity: 100%,

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% METHANOLIC AMMONIA in METHANOL with 75-25 composition method; with Flow rate=3 ml/min; Column oven temperature 40° C.; ABPR 130 bar, analysis time 5 min.

¹H NMR (400 MHz, D₂O) δ 7.73 (dd, J=8.9, 2.6 Hz, 1H), 7.67 (d, J=2.5 Hz, 1H), 7.33 (d, J=8.9 Hz, 1H), 4.62-4.54 (m, 2H), 4.40 (q, J=7.0 Hz, 2H), 3.77 (d, J=9.1 Hz, 2H), 3.24 (dd, J=13.1, 5.9 Hz, 2H), 3.14 (dd, J=24.4, 11.2 Hz, 2H), 2.46 (s, 1H), 2.25 (t, J=12.0 Hz, 2H), 2.01 (s, 1H), 1.67-1.59 (m, 3H), 1.53 (d, J=9.8 Hz, 1H).

### Experimental Protocol for Compound 8

### Synthetic Scheme

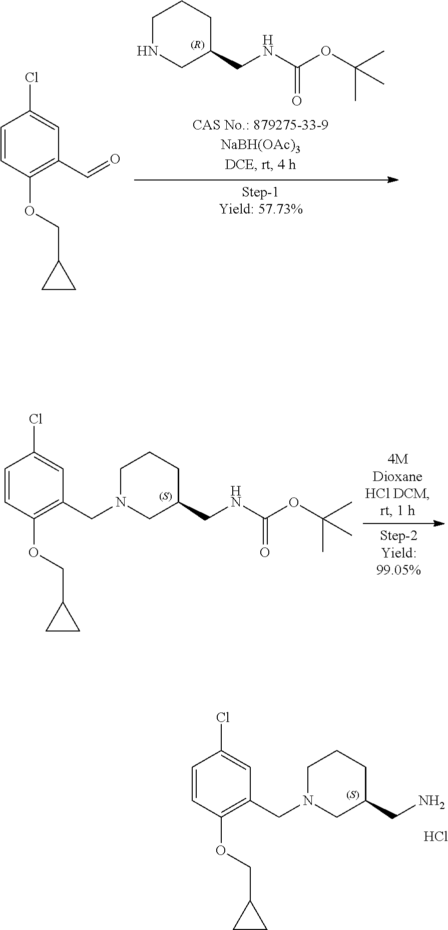

### Step-1: Synthesis of tert-butyl (S)-((1-(5-chloro-2-(cyclopropylmethoxy)benzyl)piperidin-3-yl)methyl) carbamate

### Procedure

To a stirred solution of 5-chloro-2-(cyclopropylmethoxy)benzaldehyde (0.25 g, 1.18 mmol) in DCE (5 mL) at room temperature, tert-butyl (R)-(piperidin-3-ylmethyl)carbamate (0.305 g, 1.42 mmol) was added. The reaction mixture was stirred at room temperature for 2 h. Sodium triacetoxyborohydride (0.750 g, 3.54 mmol) was added to the reaction mixture in small portions at 0° C. The reaction was stirred at room temperature for 4 h. The progress of the reaction was monitored by TLC and LCMS analysis. After completion of the reaction, the reaction mixture was poured into a saturated solution of sodium bicarbonate (50 mL) and extracted with CH₂Cl₂ (3×100 mL). The combined organic fractions were washed with water (100 mL), dried over sodium sulphate and concentrated under reduced pressure. The crude material was purified by flash column chromatography (SiO₂; 3% MeOH/CH₂Cl₂) to provide tert-butyl (S)-((1-(5-chloro-2-(cyclopropylmethoxy)benzyl)piperidin-3-yl)methyl)carbamate (0.280 g, 57.73% yield) as a colorless liquid.

LCMS [ESI, M+1]: 408.86 (RT: 1.702 min, Purity: 99.48%)

Chiral HPLC: RT: 4.15 min, Purity: 98.18%

¹H NMR (400 MHz, d₆-DMSO): δ 7.28 (d, J=2.5 Hz, 1H), 7.21 (dd, J=8.7, 2.7 Hz, 1H), 6.95 (d, J=8.8 Hz, 1H), 6.81 (t, J=5.8 Hz, 1H), 3.89-3.76 (m, 2H), 3.41 (d, J=15.3 Hz, 2H), 2.83 (d, J=13.4 Hz, 1H), 2.71 (dd, J=22.9, 7.2 Hz, 3H), 1.63 (dd, J=17.7, 10.5 Hz, 4H), 1.35 (s, 9H), 1.22 (d, J=14.3 Hz, 4H), 0.60-0.51 (m, 2H), 0.32 (t, J=4.6 Hz, 2H).

### Step2: (S)-(1-(5-chloro-2-(cyclopropylmethoxy)benzyl)piperidin-3-yl)methanamine hydrochloride (Compound 8)

### Procedure

To a stirred solution of tert-butyl (S)-((1-(5-chloro-2-(cyclopropylmethoxy)benzyl)piperidin-3-yl)methyl) carbamate (0.280 g, 0.68 mmol) in CH₂Cl₂ (2 mL) at 0° C., 4M HCl in Dioxane (1.4 mL, 5V) was added. The reaction mixture was stirred at room temperature for 1 h. The progress of the reaction was monitored by TLC and LCMS analysis. After completion of the reaction, the reaction mixture was concentrated under reduced pressure to obtain crude material which was triturated with diethyl ether to provide (S)-(1-(5-chloro-2-(cyclopropylmethoxy)benzyl)piperidin-3-yl)methanamine hydrochloride (0.209 g, 99.05%) as an off-white sticky solid.

LCMS [ESI, M+1]: 308.74 (RT: 0.952 min, Purity: 100%),

HPLC: RT: 3.952 min, Purity: 99.55%

Chiral HPLC: RT: 4.83 min, Purity: 97.53%

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: METHANOL with 75-25 composition method; with Flow rate=3 ml/min; Column oven temperature 40° C.; ABPR 130 bar, analysis time 10 min.

¹H NMR (400 MHz, CD₃OD): δ 7.61 (s, 1H), 7.47 (dd, J=2.4 Hz, 1H), 7.11 (d, J=8.8 Hz, 1H), 4.38 (s, 2H), 3.98 (d, J=7.2 Hz, 2H), 3.65-3.57 (m, 2H), 3.08-2.99 (m, 2H), 2.95-2.86 (m, 2H), 3.37 (s, 1H), 2.04 (m, 2H), 1.92 (m, 1H), 1.40-1.31 (m, 2H), 0.71-0.66 (m, 2H), 0.43-0.39 (m, 2H)

### Experimental Protocol for Compound 9

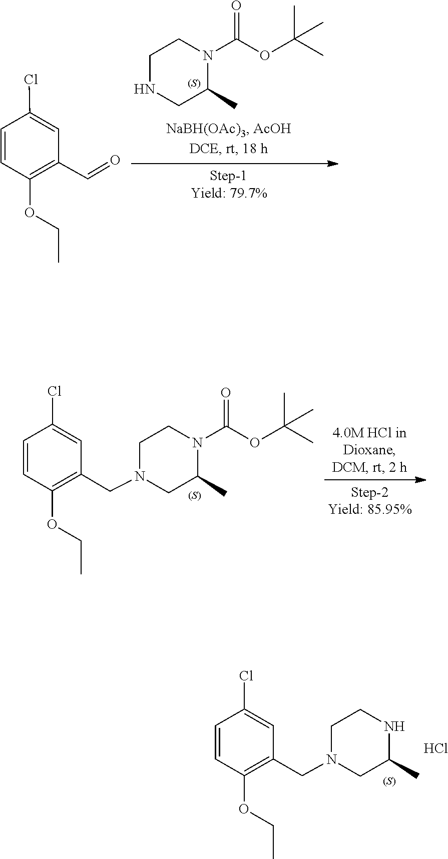

### Step-1: Synthesis of tert-butyl (S)-4-(5-chloro-2-ethoxybenzyl)-2-methylpiperazine-1-carboxylate

### Procedure

To a stirred solution of 5-chloro-2-ethoxybenzaldehyde (0.250 g, 1.35 mmol) in DCE (5 mL) at room temperature, tert-butyl (S)-2-methylpiperazine-1-carboxylate (0.325 g, 1.62 mmol) and acetic acid (0.012 mL, 0.05V) were added. The reaction mixture was stirred at room temperature for 2 h. Sodium triacetoxyborohydride (0.860 g, 4.06 mmol) was added portion-wise to the reaction mixture at 0° C. The reaction mixture was stirred at room temperature for 16 h. The progress of the reaction was monitored by TLC and LCMS analysis. After completion of reaction, the reaction mixture was poured into saturated solution of sodium bicarbonate (6 mL) and extracted with CH₂Cl₂ (3×5 mL). The combined organic fractions were washed with water (2×3 mL), dried over sodium sulfate and concentrated under reduced pressure. The crude material was purified by flash column chromatography (SiO₂; 85% ethyl acetate in hexane) to provide tert-butyl (S)-4-(5-chloro-2-ethoxybenzyl)-2-methylpiperazine-1-carboxylate (0.395 g, 79.07% yield) as a light yellow liquid.

LCMS [ESI, M+1]:369.1 (RT: 1.567 min, Purity: 92.30%),

¹H NMR (400 MHz, d₆-DMSO): δ 7.36 (d, J=2.6 Hz, 1H), 7.24 (dt, J=8.3, 4.1 Hz, 1H), 6.97 (t, J=7.7 Hz, 1H), 4.07 (d, J=7.9 Hz, 1H), 4.02 (dd, J=13.6, 6.7 Hz, 2H), 3.68 (d, J=13.1 Hz, 1H), 3.44 (s, 2H), 3.01 (t, J=11.6 Hz, 1H), 2.73 (t, J=16.9 Hz, 1H), 2.59 (t, J=13.2 Hz, 1H), 2.07 (dd, J=11.3, 3.7 Hz, 1H), 1.95 (dd, J=11.7, 3.1 Hz, 1H), 1.46-1.34 (m, 9H), 1.34-1.25 (m, 3H), 1.18 (t, J=6.8 Hz, 3H).

### Step-2: Synthesis of (S)-1-(5-chloro-2-ethoxybenzyl)-3-methylpiperazine hydrochloride (Compound 9)

### Procedure

To a stirred solution of tert-butyl (S)-4-(5-chloro-2-ethoxybenzyl)-2-methylpiperazine-1-carboxylate (0.395 g, 1.07 mmol) in CH₂Cl₂ (3.9 mL) at 0° C., 4M HCl in Dioxane (1.95 mL, 5V) was added. The reaction mixture was stirred at room temperature for 2 h. The progress of the reaction was monitored by TLC and LCMS analysis. After completion of reaction, the reaction mixture was concentrated under reduced pressure. The crude material was purified by trituration with diethyl ether to provide (S)-1-(5-chloro-2-ethoxybenzyl)-3-methylpiperazine hydrochloride (0.280 g, 85.95% yield) as an off-white solid.

LCMS [ESI, M+1]: 269.1 (RT: 0.949 min, Purity: 100%),

HPLC: RT: 3.645 min, Purity: 98.03%,

Chiral HPLC: RT: 1.87, Purity: 100%

Instrument Name: Waters SFC Investigator Chromatographic separation was conducted with Waters SFC Investigator system with 2998 PDA detector. The column used was CHIRALPAK® IA (250×4.6 mm; 5 ␣m) and the compounds were eluted with, Mobile Phase A: Liq·CO2, Mobile Phase B: 0.1% METHANOLIC AMMONIA in METHANOL with 60-40 composition method; with Flow rate=3 ml/min; Column oven temperature 40° C.; ABPR 130 bar, analysis time 5 min.

¹H NMR (400 MHz, CD₃OD): δ 7.61 (d, J=2.6 Hz, 1H), 7.49 (dd, J=8.9, 2.6 Hz, 1H), 7.15 (d, J=8.9 Hz, 1H), 4.45 (s, 2H), 4.20 (q, J=7.0 Hz, 2H), 3.82 (s, 1H), 3.74 (t, J=11.6 Hz, 3H), 3.55 (t, J=13.1 Hz, 1H), 3.41 (dd, J=25.9, 13.1 Hz, 1H), 3.26 (d, J=12.8 Hz, 1H), 1.49 (t, J=7.0 Hz, 3H), 1.42 (dd, J=15.8, 6.8 Hz, 3H).

### Experimental Protocol for Compound 10

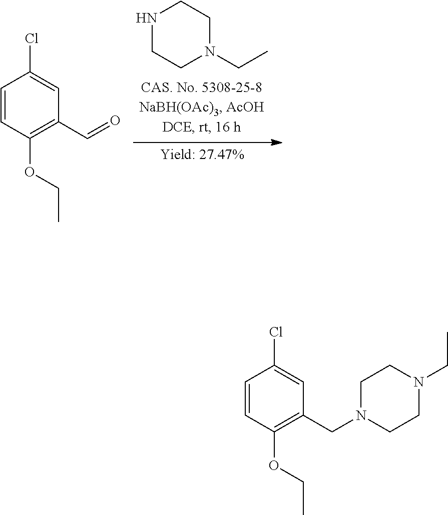

### Synthesis of 1-(5-chloro-2-ethoxybenzyl)-4-ethylpiperazine (Compound 10)

To a stirred solution of 5-chloro-2-ethoxybenzaldehyde (0.120 g, 0.65 mmol) in DCE (2.4 mL, 20V) at room temperature, 1-ethylpiperazine (0.089 g, 0.78 mmol) and acetic acid (0.001 g, 0.0325 mmol) were added. The reaction mixture was stirred at room temperature for 2 h. Sodium triacetoxyborohydride (0.414 g, 1.95 mmol) was added to the reaction mixture portion-wise at 0° C. The reaction was stirred at room temperature for 16 h. The progress of the reaction was monitored by TLC and LCMS analysis. After completion of the reaction, the reaction mixture was poured into a saturated solution of sodium bicarbonate (15 mL) and extracted with CH₂Cl₂ (3×10 mL). The combined organic fractions were washed with water (15 mL), dried over sodium sulfate and concentrated under reduced pressure. The crude material was purified by flash column chromatography (SiO₂; 30% ethyl acetate in hexane) to provide 1-(5-chloro-2-ethoxybenzyl)-4-ethylpiperazine (0.050 g, 27.47% yield) as light yellow liquid.

LCMS [ESI, M+1]:283.1 (RT: 1.023 min, Purity: 100%),

HPLC: RT: 3.672 min, Purity: 100%

¹H NMR (400 MHz, d₆-DMSO): δ 7.21 (d, J=2.4 Hz, 1H), 7.15 (dd, J=8.7, 2.6 Hz, 1H), 6.89 (d, J=8.8 Hz, 1H), 3.93 (q, J=6.9 Hz, 2H), 3.35 (s, 2H), 2.31 (m, 6H), 2.21 (dd, J=14.4, 7.2 Hz, 4H), 1.23 (t, J=6.9 Hz, 3H), 0.89 (t, J=7.2 Hz, 3H).

### Experimental Protocol for Compound 11

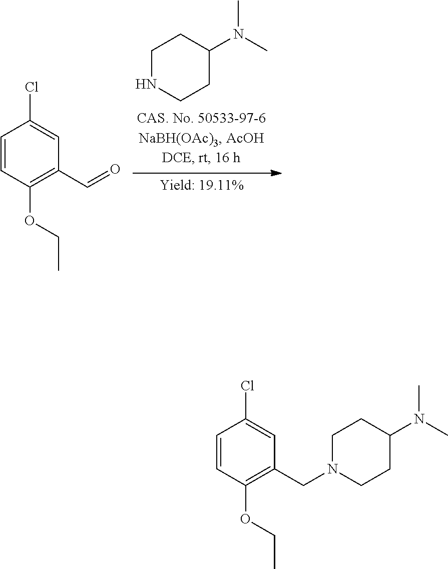

### Synthesis of 1-(5-chloro-2-ethoxybenzyl)-N,N-dimethylpiperidin-4-amine (Compound 11)

To a stirred solution of 5-chloro-2-ethoxybenzaldehyde (0.12 g, 0.65 mmol) in DCE (2 mL) at room temperature, N,N-dimethylpiperidin-4-amine (0.1 g, 0.78 mmol) and acetic acid (0.006 mL, 0.05V) were added. The reaction mixture was stirred at room temperature for 2 h. Sodium triacetoxyborohydride (0.41 g, 1.95 mmol) was added portion-wise to the reaction mixture at 0° C. The reaction mixture was allowed to stir at room temperature for 16 h. The progress of the reaction was monitored by TLC and LCMS analysis. After completion of the reaction, the reaction mixture was diluted with CH₂Cl₂ (5 mL) and washed with sat. NaHCO₃ solution (5 mL) and water (5 mL). The organic layer was dried over anhydrous sodium sulphate and concentrated under reduced pressure. The crude material was purified by flash column chromatography (SiO₂; 15% MeOH/CH₂Cl₂) to provide 1-(5-chloro-2-ethoxybenzyl)-N,N-dimethylpiperidin-4-amine (0.0369 g, 19.11% yield) as a light brown liquid.

LCMS [ESI, M+1]: 297.11 (RT: 0.771 min, Purity: 100%),

HPLC: RT: 3.592 min, Purity: 99.66%

¹H-NMR (400 MHz, d₆-DMSO): δ 7.29 (d, J=2.4 Hz, 1H), 7.24-7.21 (dd, J=2.8 Hz, 8.8 Hz, 1H), 6.96 (d, J=8.8 Hz, 1H), 4.00 (q, J=7.2 Hz, 2H), 3.43 (s, 2H), 2.84 (d, J=11.2 Hz, 2H), 2.29 (s, 6H), 1.97 (t, J=11.2 Hz, 2H), 1.90 (s, 1H), 1.76 (d, J=11.6 Hz, 2H), 1.46-1.42 (m, 2H), 1.31 (t, J=7.2 Hz, 3H).

### Experimental Protocol for Compound 12

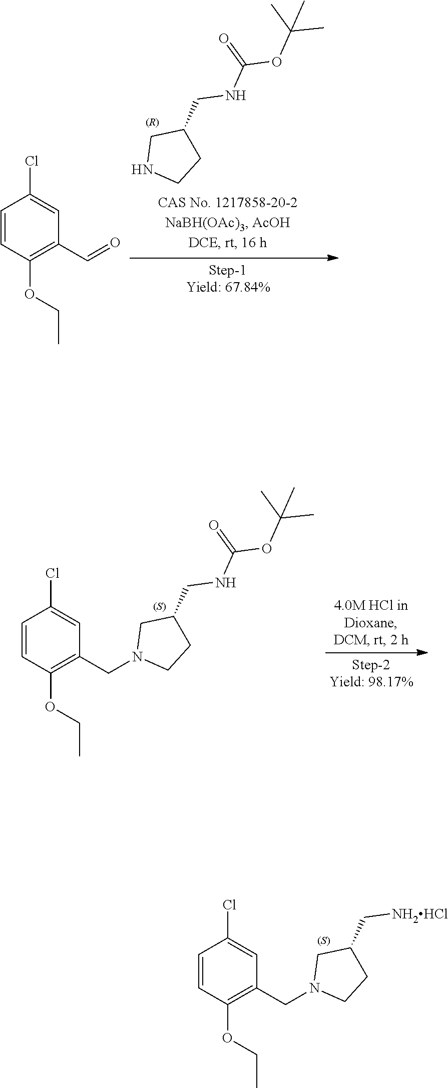

### Step-1: Synthesis of tert-butyl (S)-((1-(5-chloro-2-ethoxybenzyl)pyrrolidin-3-yl)methyl) carbamate

### Procedure

To a stirred solution of 5-chloro-2-ethoxybenzaldehyde (0.10 g, 0.54 mmol) in DCE (2 mL) at room temperature, tert-butyl (R)-(pyrrolidin-3-ylmethyl)carbamate hydrochloride (0.15 g, 0.63 mmol) was added. The reaction mixture was stirred at room temperature for 2 h. Sodium triacetoxyborohydride (0.34 g, 1.60 mmol) was added portion-wise to the reaction mixture at 0° C. The reaction mixture was allowed to stir at room temperature for 16 h. The progress of the reaction was monitored by TLC and LCMS analysis. After completion of the reaction, the reaction mixture was diluted with CH₂Cl₂ (10 mL) and poured into cold water (10 mL). The organic layer was washed with sat. NaHCO₃ solution (10 mL), dried over anhydrous sodium sulfate and concentrated under reduced pressure. The crude material was purified by flash column chromatography (neutral alumina; 0-100% EtOAc/hexane as mobile phase) to provide tert-butyl (S)-((1-(5-chloro-2-ethoxybenzyl) pyrrolidin-3-yl)methyl) carbamate (0.135 g, 67.84% yield) as a yellow oil.

LCMS [ESI, M+1]: 369.0 (RT: 1.428 min, Purity: 98.62%),

¹H-NMR (400 MHz, d₆-DMSO): δ 7.28 (d, J=2.4 Hz, 1H), 7.22-7.20 (dd, J=2.8 Hz, 8.8 Hz, 1H), 6.95 (d, J=8.8 Hz, 1H), 6.87 (t, J=5.0 Hz, 1H), 4.01 (q, J=6.8 Hz, 2H), 3.51 (s, 2H), 2.88 (t, J=6.0 Hz, 2H), 2.24-2.21 (m, 3H), 1.81-1.80 (m, 2H), 1.36-1.29 (m, 14H).

### Step-2: (S)-(1-(5-chloro-2-ethoxybenzyl)pyrrolidin-3-yl)methanamine hydrochloride (Compound 12)

### Procedure

To a stirred solution of tert-butyl (S)-((1-(5-chloro-2-ethoxybenzyl) pyrrolidin-3-yl)methyl) carbamate (0.1 g, 0.27 mmol) in CH₂Cl₂ (1.0 mL) at 0° C., 4.0 M HCl in Dioxane (0.5 mL, 5V) at was added. The resulting reaction mixture was stirred at room temperature for 2 h. The progress of the reaction was monitored by TLC and LCMS analysis. After completion of the reaction, the reaction mixture was concentrated under reduced pressure and the crude material purified by trituration using diethyl ether (5 ml) to provide (S)-(1-(5-chloro-2-ethoxybenzyl)pyrrolidin-3-yl)methanamine hydrochloride (0.071 g, 98.17% yield) as a white solid.

LCMS [ESI, M+1]: 269.0 (RT: 0.770 min, Purity: 98.76%),

HPLC Purity: RT: 3.220 min, Purity: 98.05%

Chiral HPLC: 1.78, Purity: 100%,

Instrument Name: Waters SFC Investigator

Chromatographic separation was conducted with Waters SFC Investigator system with 2998 PDA detector. The column used was CHIRALPAK® IG (250×4.6 mm; 5 ␣m) and the compounds were eluted with, Mobile Phase A: Liq·CO2, Mobile Phase B: 0.1% METHANOLIC AMMONIA in METHANOL with 75-25 composition method; with Flow rate=3 ml/min; Column oven temperature 40° C.; ABPR 130 bar, analysis time 9 min.

¹H-NMR (400 MHz, CD₃OD): δ 7.56 (bs, 1H), 7.48-7.45 (dd, J=2.4 Hz, 8.8 Hz, 1H), 7.12 (d, J=9.2 Hz, 1H), 4.49-4.43 (m, 2H), 4.20 (q, J=6.8 Hz, 2H), 3.78-3.73 (m, 1H), 3.67-3.60 (m, 1H), 3.57-3.38 (m, 1H), 3.16-3.06 (m, 3H), 2.94-2.74 (m, 1H), 2.43-2.31 (m, 1H), 2.03-1.87 (m, 1H), 1.49 (t, J=6.8 Hz, 3H).

### Experimental Protocol for Compound 13

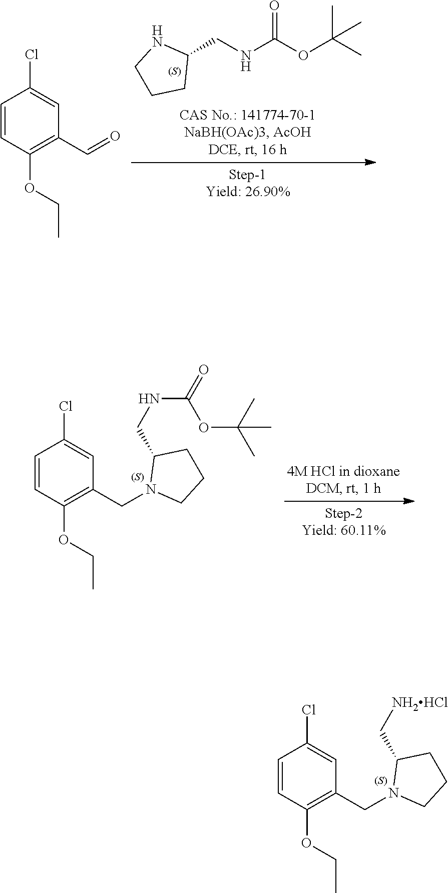

### Step-1: tert-butyl (S)-((1-(5-chloro-2-ethoxybenzyl)pyrrolidin-2-yl)methyl)carbamate

### Procedure

To a stirred solution of 5-chloro-2-ethoxybenzaldehyde (0.080 g, 0.43 mmol) in DCE (1.6 mL, 20V) at room temperature, tert-butyl (S)-(pyrrolidin-2-ylmethyl)carbamate (0.104 g, 0.52 mmol) and acetic acid (0.001 g, 0.02 mmol) were added. The reaction mixture was stirred at room temperature for 2 h. Sodium triacetoxyborohydride (0.27 g, 1.30 mmol) was added to the reaction mixture portion-wise at 0° C. The reaction mixture was stirred at room temperature for 16 h. The progress of the reaction was monitored by TLC and LCMS analysis. After completion of the reaction, the reaction mixture was poured into a saturated solution of sodium bicarbonate (15 mL) and extracted with CH₂Cl₂ (3×10 mL). The combined organic fractions were washed with water (15 mL), dried over sodium sulfate, and concentrated under reduced pressure. The crude material was purified by flash column chromatography (SiO₂; 0-15% CH₃OH in CH₂Cl₂) to provide tert-butyl (S)-((1-(5-chloro-2-ethoxy benzyl)pyrrolidin-2-yl)methyl)carbamate (0.043 g, 26.90% yield) as a light yellow liquid.

LCMS [ESI, M+1]:369.1 (RT: 1.612 min, Purity: 100%),

### Step-2: (S)-(1-(5-chloro-2-ethoxybenzyl)pyrrolidin-2-yl)methanamine hydrochloride (Compound 13)

### Procedure

To a stirred solution of tert-butyl (S)-((1-(5-chloro-2-ethoxybenzyl)pyrrolidin-2-yl)methyl) carbamate (0.040 g, 0.10 mmol) in CH₂Cl₂ (0.4 mL, 10V) at room temperature, 4M Hydrochloric acid in dioxane solution (0.2 mL, 5V) was added. The reaction mixture was stirred at room temperature for 1 h. The progress of the reaction was monitored by TLC and LCMS analysis. After completion of reaction, the reaction mixture was concentrated under reduced pressure. The crude material was purified by trituration using CH₂Cl₂ to provide (S)-(1-(5-chloro-2-ethoxybenzyl)pyrrolidin-2-yl)methanamine hydrochloride (0.017 g, 60.11% yield) as a light yellow sticky solid LCMS [ESI, M+1]: 269.05, (RT: 0.783 min, Purity: 98.82%),

HPLC: RT: 3.120, Purity: 98.90%

¹H NMR (400 MHz, CD₃OD): δ 7.53 (d, J=2.5 Hz, 1H), 7.41 (dd, J=8.9, 2.6 Hz, 1H), 7.06 (d, J=8.9 Hz, 1H), 4.61 (d, J=12.3 Hz, 1H), 4.22-4.07 (m, 3H), 3.78 (s, 1H), 3.58 (d, J=9.5 Hz, 1H), 3.43 (d, J=14.0 Hz, 1H), 3.39-3.27 (m, 2H), 2.44-2.35 (m, 1H), 2.18-2.10 (m, 1H), 2.09-2.01 (m, 1H), 1.95-1.89 (m, 1H), 1.43 (t, J=7.0 Hz, 3H).

### Experimental Protocol for Compound 14

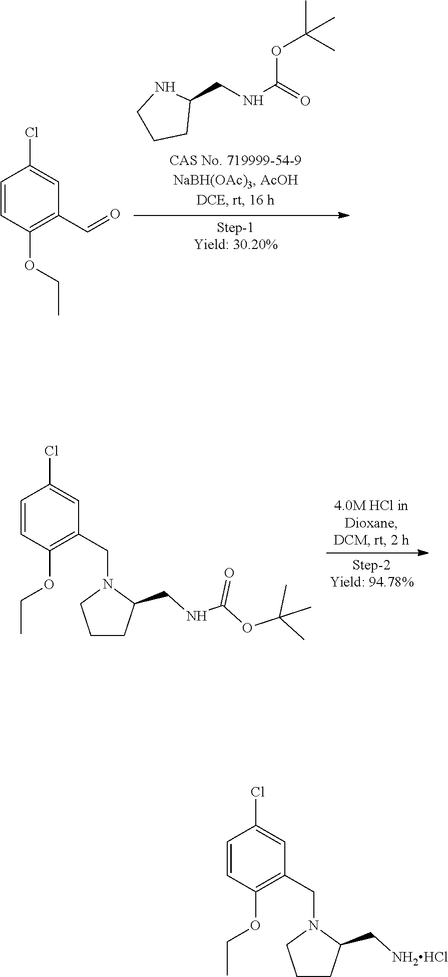

### Step-1: Synthesis of tert-butyl (R)-((1-(5-chloro-2-ethoxybenzyl)pyrrolidin-2-yl)methyl) carbamate

### Procedure

To a stirred solution of 5-chloro-2-ethoxybenzaldehyde (0.15 g, 0.81 mmol) in DCE (3 mL) at room temperature, added tert-butyl (R)-(pyrrolidin-2-ylmethyl)carbamate (0.19 g, 0.97 mmol) and acetic acid (0.008 mL, 0.05V) were added. The reaction mixture was stirred at room temperature for 2 h. Sodium triacetoxyborohydride (0.51 g, 2.43 mmol) was added portion-wise to the reaction mixture at 0° C. The reaction mixture was allowed to stir at room temperature for 16 h. The progress of the reaction was monitored by TLC and LCMS analysis. After completion of the reaction, the reaction mixture was diluted with CH₂Cl₂ (15 mL) and washed with sat. NaHCO₃ solution (15 mL) and water (15 mL). The organic layer was dried over anhydrous sodium sulfate and concentrated under reduced pressure. The crude material was purified by flash column chromatography (neutral alumina; 0-100% EtOAc/hexane) to provide tert-butyl (R)-((1-(5-chloro-2-ethoxybenzyl)pyrrolidin-2-yl)methyl) carbamate (0.090 g, 30.20% yield) as a yellow oil.

LCMS [ESI, M+1]: 369.1 (RT: 1.553 min, Purity: 98.72%),

¹H-NMR (400 MHz, CD₃OD): δ 7.30 (s, 1H), 7.22-7.19 (dd, J=2.4 Hz, 8.8 Hz, 1H), 6.93 (d, J=8.8 Hz, 1H), 6.68 (d, J=8.0 Hz, 1H), 4.11-4.01 (m, 3H), 3.09-3.04 (m, 1H), 2.94 (bs, 1H), 2.65 (bs, 1H), 2.29-2.25 (m, 1H), 1.95-1.88 (m, 1H), 1.71-1.68 (m, 2H), 1.62-1.59 (m, 2H), 1.44-1.41 (m, 12H).

### Step-2: Synthesis of (R)-(1-(5-chloro-2-ethoxybenzyl)pyrrolidin-2-yl)methanamine hydrochloride (Compound 14)

### Procedure

To a stirred solution of tert-butyl (R)-((1-(5-chloro-2-ethoxybenzyl)pyrrolidin-2-yl)methyl) carbamate (0.09 g, 0.24 mmol) in CH₂Cl₂ (1.0 mL) at 0° C., 4.0 M HCl in Dioxane (0.5 mL, 5V) was added. The reaction mixture was stirred at room temperature for 2 h. The progress of the reaction was monitored by TLC and LCMS analysis. After completion of the reaction, the reaction mixture was concentrated under reduced pressure and the crude material was purified by trituration using diethyl ether to provide (R)-(1-(5-chloro-2-ethoxybenzyl)pyrrolidin-2-yl)methanamine hydrochloride (0.0621 g, 94.78% yield) as a white solid.

LCMS [ESI, M+1]: 269.1 (RT: 0.770 min, Purity: 96.26%),

HPLC Purity: RT: 3.420 min, Purity: 100.0%

¹H-NMR (400 MHz, CD₃OD): δ 7.62 (d, J=2.4 Hz, 1H), 7.49-7.47 (dd, J=2.4 Hz, 8.8 Hz, 1H), 7.13 (d, J=8.8 Hz, 1H), 4.70 (d, J=12.8 Hz, 1H), 4.3 (d, J=12.8 Hz, 1H), 4.25-4.17 (m, 2H), 3.91-3.89 (m, 1H), 3.69-3.65 (dd, J=4.0 Hz, 12.8 Hz, 1H), 3.59-3.54 (m, 1H), 3.52-3.37 (m, 2H), 2.52-2.47 (m, 1H), 2.25-2.12 (m, 2H), 2.07-2.00 (m, 1H), 1.51 (t, J=6.8 Hz, 3H).

### Experimental Protocol for Compound 15

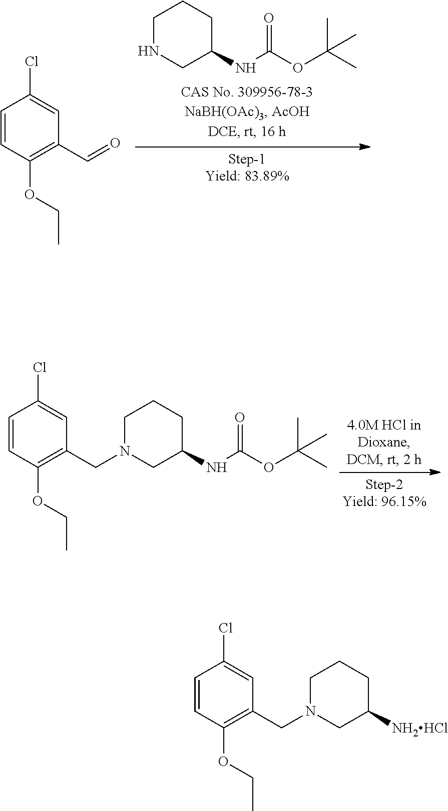

### Step-1: Synthesis of tert-butyl (R)-(1-(5-chloro-2-ethoxybenzyl)piperidin-3-yl)carbamate

### Procedure

To a stirred solution of 5-chloro-2-ethoxybenzaldehyde (0.15 g, 0.81 mmol) in DCE (3 mL) at room temperature, tert-butyl (R)-piperidin-3-ylcarbamate (0.19 g, 0.97 mmol) and acetic acid (0.008 mL, 0.05V) were added. The reaction mixture was stirred at room temperature for 2 h. Sodium triacetoxyborohydride (0.52 g, 2.45 mmol) was added portion-wise to the reaction mixture at 0° C. The reaction mixture was allowed to stir at room temperature for 16 h. The progress of the reaction was monitored by TLC and LCMS analysis. After completion of reaction, the reaction mixture was diluted with CH₂Cl₂ (20 mL) and washed with sat. NaHCO₃ solution (20 mL) and water (20 mL). The organic layer was dried over anhydrous sodium sulfate and concentrated under reduced pressure. The crude material was purified by flash column chromatography (SiO₂; 0-100% EtOAc/hexane) to provide tert-butyl (R)-(1-(5-chloro-2-ethoxybenzyl) piperidin-3-yl)carbamate (0.25 g, 83.89% yield) as a yellow oil.

LCMS [ESI, M+1]: 369.1 (RT: 1.497 min, Purity: 97.65%),

¹H-NMR (400 MHz, d₆-DMSO): δ 7.30 (d, J=2.4 Hz, 1H), 7.24-7.21 (dd, J=2.6 Hz, 8.8 Hz, 1H), 6.96 (d, J=8.8 Hz, 1H), 6.68 (d, J=8.0 Hz, 1H), 4.00 (q, J=6.8 Hz, 2H), 3.44-3.39 (m, 3H), 3.16 (d, J=5.2 Hz, 1H), 2.75-2.73 (m, 1H), 2.61-2.58 (m, 1H), 1.93-1.90 (m, 1H), 1.80 (t, J=9.8 Hz, 1H), 1.67-1.59 (m, 2H), 1.44-1.29 (m, 13H).

### Step-2: Synthesis of (R)-1-(5-chloro-2-ethoxybenzyl)piperidin-3-amine hydrochloride (Compound 15)

### Procedure

To a stirred solution of tert-butyl (R)-(1-(5-chloro-2-ethoxybenzyl)piperidin-3-yl)carbamate (0.25 g, 0.67 mmol) in CH₂Cl₂ (2.5 mL) at 0° C., 4.0 M HCl in Dioxane (1.25 mL, 5V) was added. The reaction mixture was then stirred at room temperature for 2 h. The progress of the reaction was monitored by TLC and LCMS analysis. After completion of reaction, the reaction mixture was concentrated under reduced pressure. The crude material was purified by trituration using diethyl ether to provide (R)-1-(5-chloro-2-ethoxybenzyl)piperidin-3-amine hydro chloride (0.175 g, 96.15% yield) as a white solid.

LCMS [ESI, M+1]: 269.1 (RT: 0.775 min, Purity: 100%),

HPLC Purity: RT: 3.459 min, Purity: 100.0%

¹H-NMR (400 MHz, CD₃OD): δ 7.62 (d, J=2.4 Hz, 1H), 7.50-7.47 (dd, J=2.4 Hz, 8.8 Hz, 1H), 7.14 (d, J=8.8 Hz, 1H), 4.48-4.40 (m, 2H), 4.20 (q, J=6.8 Hz, 2H), 3.67-3.62 (m, 3H), 3.11-3.09 (m, 2H), 2.21-2.11 (m, 2H), 1.98-1.95 (m, 1H), 1.68-1.65 (m, 1H), 1.49 (t, J=6.8 Hz, 3H).

### Experimental Protocol for Compound 16

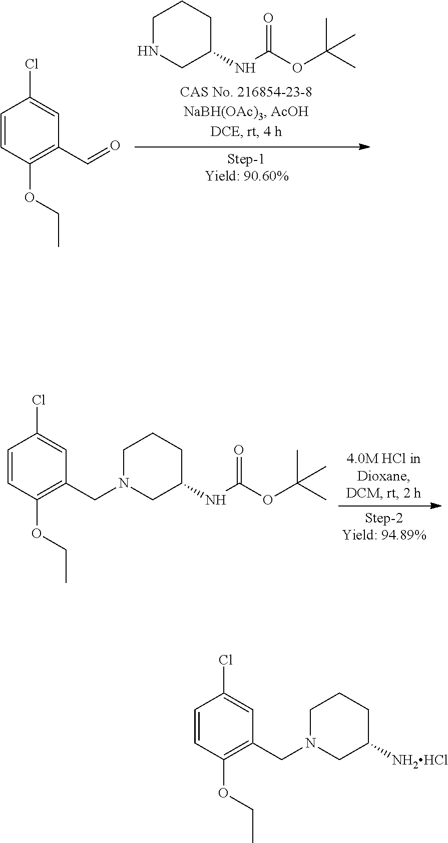

### Step-1: Synthesis of tert-butyl (S)-(1-(5-chloro-2-ethoxybenzyl)piperidin-3-yl)carbamate

### Procedure

To a stirred solution of 5-chloro-2-ethoxybenzaldehyde (0.15 g, 0.81 mmol) in DCE (3 mL) at room temperature, tert-butyl (S)-piperidin-3-ylcarbamate (0.19 g, 0.97 mmol) and acetic acid (0.008 mL, 0.05V) were added. The reaction mixture was stirred at room temperature for 2 h. Sodium triacetoxyborohydride (0.52 g, 2.45 mmol) was added portion-wise added to the reaction mixture at 0° C. The reaction mixture was allowed to stir at room temperature for 2 h. The progress of the reaction was monitored by TLC and LCMS analysis. After completion of the reaction, the reaction mixture was diluted with CH₂Cl₂ (20 mL) and washed with sat. NaHCO₃ solution (20 mL) and water (20 mL). The organic layer was dried over anhydrous sodium sulphate and concentrated under reduced pressure. The crude material was purified by flash column chromatography (SiO₂; 0-100% EtOAc/hexane) to provide tert-butyl (S)-(1-(5-chloro-2-ethoxybenzyl) piperidin-3-yl)carbamate (0.27 g, 90.60% yield) as a yellow oil.

LCMS [ESI, M+1]: 369.1 (RT: 1.448 min, Purity: 97.13%),

¹H-NMR (400 MHz, d₆-DMSO): δ 7.30 (d, J=2.8 Hz, 1H), 7.24-7.21 (dd, J=2.8 Hz, 8.8 Hz, 1H), 6.97 (d, J=8.8 Hz, 1H), 6.69 (d, J=8.0 Hz, 1H), 4.00 (q, J=6.8 Hz, 2H), 3.48-3.37 (m, 3H), 2.75-2.73 (m, 1H), 2.61-2.56 (m, 1H), 1.93-1.88 (m, 1H), 1.82-1.78 (m, 1H), 1.68-1.59 (m, 2H), 1.46-1.23 (m, 13H), 1.14-1.06 (m, 1H).

### Step-2: Synthesis of (S)-1-(5-chloro-2-ethoxybenzyl)piperidin-3-amine hydrochloride (Compound 16)

### Procedure

To a stirred solution of tert-butyl (S)-(1-(5-chloro-2-ethoxybenzyl)piperidin-3-yl)carbamate (0.27 g, 0.73 mmol) in CH₂Cl₂ (3.0 mL) at 0° C., 4.0 M HCl in Dioxane (1.35 mL, 5V) was added. The reaction mixture was stirred at room temperature for 2 h. The progress of the reaction was monitored by TLC and LCMS analysis. After completion of the reaction, the reaction mixture was concentrated under reduced pressure. The crude material was purified by trituration using diethyl ether to provide (S)-1-(5-chloro-2-ethoxybenzyl)piperidin-3-amine hydrochloride (0.186 g, 94.89% yield) as a white solid.

LCMS [ESI, M+1]: 269.1 (RT: 0.781 min, Purity: 97.90%),

Chiral HPLC: RT: 4.04, Purity: 100%,

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% METHANOLIC AMMONIA in METHANOL-ACETONITRILE (50-50) with 80-20 composition method; with Flow rate=3 ml/min; Column oven temperature 40° C.; ABPR 130 bar, analysis time 8 min.

¹H-NMR (400 MHz, CD₃OD): δ 7.62 (s, 1H), 7.49 (d, J=8.4 Hz, 1H), 7.14 (d, J=8.8 Hz, 1H), 4.44-4.40 (m, 2H), 4.21-4.19 (m, 2H), 3.67-3.62 (m, 3H), 3.11 (bs, 2H), 2.21-2.11 (m, 2H), 1.98-1.95 (m, 1H), 1.68-1.65 (m, 1H), 1.51-1.49 (m, 3H).

### Experimental Protocol for Compound 17

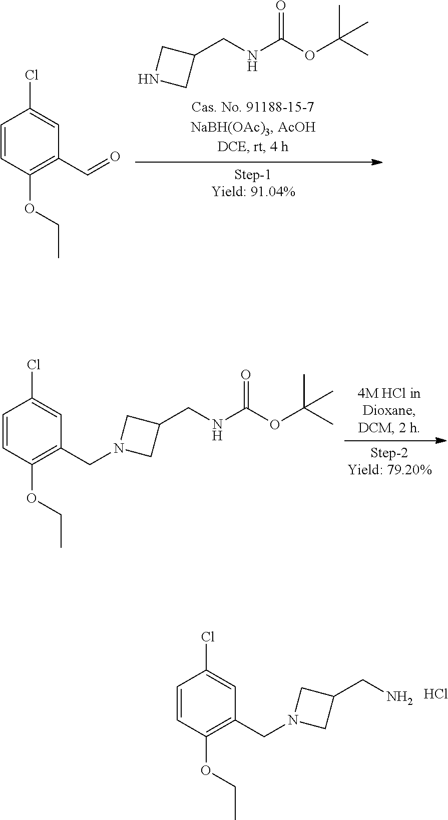 [SMILES: CCOc1ccc(Cl)cc1C=O ; CC(C)(C)OC(=O)NCC1CNC1 ; CCOc1ccc(Cl)cc1CN1CC(CNC(=O)OC(C)(C)C)C1 ; CCOc1ccc(Cl)cc1CN1CC(CN)C1 ; Cl]

### Step-1: Synthesis of tert-butyl ((1-(5-chloro-2-ethoxybenzyl)azetidin-3-yl)methyl)carbamate

### Procedure

To a stirred solution of 5-chloro-2-ethoxybenzaldehyde (0.200 g, 1.08 mmol) in DCE (4 mL) at room temperature, tert-butyl (azetidin-3-ylmethyl) carbamate Hydrochloride (0.201 g, 1.08 mmol) was added. The reaction mixture was stirred at room temperature for 2 h. Sodium triacetoxyborohydride (0.688 g, 3.24 mmol) was added portion-wise at 0° C. After addition the reaction was stirred at room temperature for 4 h. The progress of the reaction was monitored by TLC and LCMS analysis. After completion of the reaction, the reaction mixture was poured into a saturated solution of sodium bicarbonate (5 mL) and extracted with CH₂Cl₂ (3×4 mL). The combined organic fractions were washed with water (2×3 mL), dried over sodium sulfate and concentrated under reduced pressure. The crude material was purified by flash column chromatography (SiO₂; 0-100% ethyl acetate in hexane) to provide tert-butyl ((1-(5-chloro-2-ethoxybenzyl)azetidin-3-yl)methyl)carbamate (0.350 g, 91.04% yield) as a light yellow liquid.

LCMS [ESI, M−56]:298.1 (RT: 1.421 min, Purity: 99.34%),

¹H NMR (400 MHz, d₆-DMSO): δ 7.33-7.17 (m, 2H), 6.96 (d, J=8.1 Hz, 1H), 4.11-3.95 (m, 2H), 3.49 (s, 2H), 3.22 (t, J=7.2 Hz, 2H), 3.08 (dd, J=35.8, 29.5 Hz, 2H), 2.91 (t, J=6.3 Hz, 2H), 1.92 (s, 2H), 1.37 (d, J=13.0 Hz, 9H), 1.34 (t, J=6.9 Hz, 3H).

### Step-2: Synthesis of (1-(5-chloro-2-ethoxybenzyl)azetidin-3-yl)methanamine hydrochloride (Compound 17)

### Procedure

To a stirred solution of tert-butyl ((1-(5-chloro-2-ethoxybenzyl)azetidin-3-yl)methyl)carbamate (0.350 g, 0.98 mmol) in CH₂Cl₂ (3.5 mL) at 0° C., 4M HCl in Dioxane (1.4 mL, 5V) was added. The reaction mixture was stirred at room temperature for 2 h. The progress of the reaction was monitored by TLC and LCMS analysis. After completion of the reaction, the reaction mixture was concentrated under reduced pressure and the crude material was purified by trituration with diethyl ether to provide (1-(5-chloro-2-ethoxybenzyl)azetidin-3-yl)methanamine hydrochloride (0.199 g, 79.20% yield) as a white solid.

LCMS [ESI, M+1]: 255.1 (RT: 0.739 min, Purity: 97.13%),

HPLC: RT: 3.466 min, Purity: 96.52%,

¹H NMR (400 MHz, CD₃OD): δ 7.42 (d, J=2.6 Hz, 1H), 7.35 (dd, J=8.9, 2.6 Hz, 1H), 7.01 (d, J=8.9 Hz, 1H), 4.36 (s, 2H), 4.21 (s, 2H), 4.16-4.00 (m, 4H), 3.29-3.23 (m, 2H), 3.14 (dd, J=15.3, 7.9 Hz, 1H), 1.45-1.35 (m, 3H).

### Experimental Protocol for Compound 18

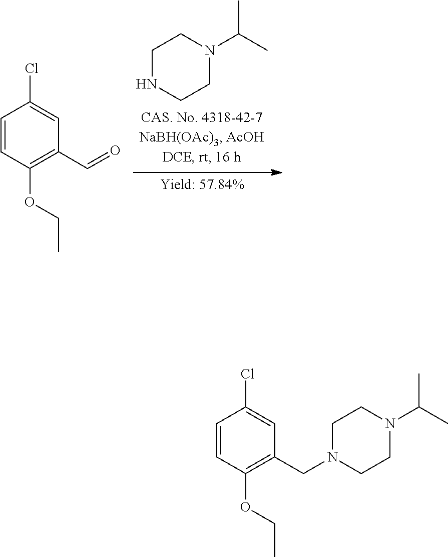

### Synthesis of 1-(5-chloro-2-ethoxybenzyl)-4-isopropylpiperazine (Compound 18)

To a stirred solution of 5-chloro-2-ethoxybenzaldehyde (0.100 g, 0.54 mmol) in DCE (2 mL) at room temperature, 1-isopropylpiperazine (0.083 g, 0.64 mmol) and acetic acid (0.005 mL, 0.05V) were added. The reaction mixture was stirred at room temperature for 2 h. Sodium triacetoxyborohydride (0.413 g, 1.95 mmol) was added portion-wise to the reaction mixture at 0° C. The reaction was stirred at room temperature for 16 h. The progress of the reaction was monitored by TLC and LCMS analysis. After completion of reaction, the reaction mixture was poured into saturated solution of sodium bicarbonate (3 mL) and extracted with CH₂Cl₂ (2×3 mL). The combined organic fractions were washed with water (3 mL), dried over sodium sulfate and concentrated under reduced pressure. The crude material was purified by flash column chromatography (60-70% ethyl acetate in hexane) to provide 1-(5-chloro-2-ethoxybenzyl)-4-isopropylpiperazine (0.093 g, 57.84% yield) as a light yellow liquid.

LCMS [ESI, M+1]:297.1 (RT: 1.094 min, Purity: 98.95%),

HPLC: RT: 3.71 min, Purity: 95.22%,

¹H NMR (400 MHz, d₆-DMSO): δ 7.29 (d, J=2.5 Hz, 1H), 7.22 (dd, J=8.7, 2.6 Hz, 1H), 6.97 (d, J=8.7 Hz, 1H), 4.00 (q, J=6.9 Hz, 2H), 3.40 (d, J=20.0 Hz, 2H), 2.63-2.54 (m, 2H), 2.41 (d, J=15.0 Hz, 7H), 1.38-1.23 (m, 3H), 0.95 (d, J=6.5 Hz, 6H).

### Experimental Protocol for Compound 19

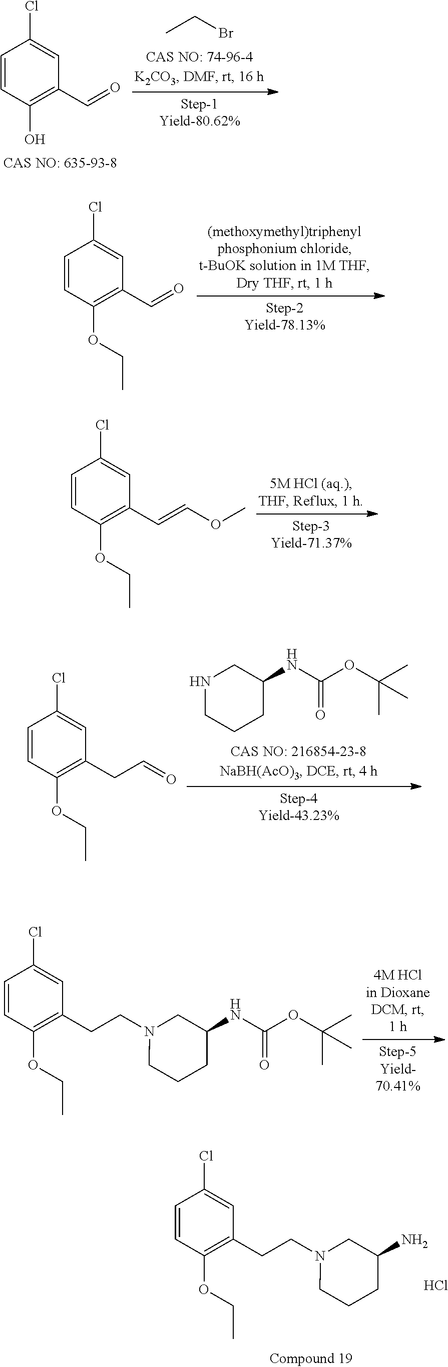

### Step-1: Synthesis of 5-chloro-2-ethoxybenzaldehyde

### Procedure

To a stirred solution of 5-chloro-2-hydroxybenzaldehyde, (0.5 g, 3.19 mmol) in DMF (5 mL, 10V) at room temperature, potassium carbonate (1.3 g, 9.58 mmol) was added. The reaction mixture was stirred at room temperature for 1 h. Bromo ethane (0.3 ml, 4.78 mmol) was then added drop-wise to the reaction mixture at 0° C. The reaction mixture was stirred at room temperature for 16 h. The progress of the reaction was monitored by TLC. After completion of the reaction, the reaction was quenched with ice cold water (30 mL). A solid precipitate was obtained, which was isolated by filtration, washed with cold water (2×10 mL), and dried under reduced pressure to provide 5-chloro-2-ethoxybenzaldehyde (0.5 g, 84.80% yield) as a white solid.

LCMS [ESI, M+1]: 185.1 (RT: 2.598 min, Purity: 100%).

### Step-2: Synthesis of (E)-4-chloro-1-ethoxy-2-(2-methoxyvinyl) benzene

### Procedure

To a stirred solution of (methoxymethyl)triphenyl phosphonium chloride, (2.3 g, 6.77 mmol) in anhydrous THF (20 mL, 40V) at 0° C., t-BuOK solution in 1M THF (12.5 mL) was added. The mixture was stirred at room temperature for 1 h. A solution of 5-chloro-2-ethoxybenzaldehyde (0.5 g, 2.70 mmol) in in THF (1 mL) was then added drop-wise to the mixture at 0° C. The reaction was stirred at room temperature for 1 h. The progress of the reaction was monitored by TLC. After completion of the reaction, the reaction mixture was concentrated under reduced pressure and the resulting crude material purified by normal phase column chromatography (neutral silica, 100% hexane) to provide (E)-4-chloro-1-ethoxy-2-(2-methoxyvinyl) benzene (0.45 g, 78.13% yield) as a light yellow liquid.

### Step-3: Synthesis of 2-(5-chloro-2-ethoxyphenyl) acetaldehyde

### Procedure

To a stirred solution of (E)-4-chloro-1-ethoxy-2-(2-methoxyvinyl) benzene (0.45 g, 1.98 mmol) in anhydrous THF (4.5 mL, 10V) at room temperature, 5M HCl solution (2.2 mL, 5V) was added. The reaction mixture was heated at reflux for 1 h. The progress of the reaction was monitored by TLC analysis (2,4-DNP stain). After completion of the reaction, the reaction mixture was poured into a saturated solution of sodium bicarbonate (30 mL) and extracted with ethyl acetate (3×15 mL). The combined organic fractions were washed with water (20 mL), dried over sodium sulphate and concentrated under reduced pressure to provide 2-(5-chloro-2-ethoxyphenyl) acetaldehyde (0.3 g, 71.37% yield) as an off-white solid.

### Step-4: Synthesis of tert-butyl (S)-(1-(5-chloro-2-ethoxyphenethyl) piperidin-3-yl)carbamate

### Procedure

To a stirred solution of 2-(5-chloro-2-ethoxyphenyl) acetaldehyde (0.25 g, 1.010 mmol) in DCE (2.5 mL, 10V) at room temperature, tert-butyl (S)-piperidin-3-ylcarbamate (0.202 g, 1.010 mmol) was added. The reaction mixture was stirred at room temperature for 1 h. Sodium triacetoxyborohydride (0.468 g, 2.22 mmol) was added portion-wise to the reaction mixture at 0° C. The reaction was stirred at room temperature for 3 h. The progress of the reaction was monitored by TLC and LCMS analysis. After completion of the reaction, the reaction mixture was poured into a saturated solution of sodium bicarbonate (30 mL) and extracted with dichloromethane (3×20 mL). The combined organic fractions were washed with water (25 mL), dried over sodium sulphate and concentrated under reduced pressure. The crude material was purified by flash column chromatography (neutral silica; 20% ethyl acetate in hexane) to provide tert-butyl (S)-(1-(5-chloro-2-ethoxyphenethyl) piperidin-3-yl)carbamate (0.25 g,% 43.23 yield) as a light yellow liquid.

LCMS [ESI, M+1]: 383.1 (RT: 1.620 min, Purity: 100%).

### Step-5: Synthesis of (S)-1-(5-chloro-2-ethoxyphenethyl)piperidin-3-amine hydrochloride (Compound 19)

### Procedure

To a stirred solution of tert-butyl (S)-(1-(5-chloro-2-ethoxyphenethyl) piperidin-3-yl)carbamate (0.25 g, 0.65 mmol) in CH₂Cl₂ (2.5 mL, 10V) at 0° C., 4M HCl in dioxane (1.5 mL, 5V) was added. The reaction mixture was stirred at room temperature for 1 h. The progress of the reaction was monitored by TLC. After completion of the reaction, the reaction mixture was concentrated under reduced pressure. The crude material was purified by reverse phase column chromatography (CH₃CN/water) to provide (S)-1-(5-chloro-2-ethoxyphenethyl)piperidin-3-amine hydrochloride (0.13 g, 70.41% yield) as a white solid.

LCMS [ESI, M+1]: 282.9 (RT: 0.917 min, Purity: 100%),

HPLC: RT: 4.59 min, Purity: 96.71%,

Chiral HPLC: RT: 2.77 min, Purity: 100%,

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% METHANOLIC AMMONIA in METHANOL-ACETONITRILE (50-50) with 5-50% from 0 to 5 min, after that remain 50% composition method; with Flow rate=4 ml/min; Column oven temperature 40° C.; ABPR 100 bar, analysis time 8 min.

¹H NMR (400 MHz, CD₃OD) δ 7.30 (t, J=5.9 Hz, 1H), 7.29-7.23 (m, 1H), 6.99 (d, J=8.7 Hz, 1H), 4.23-4.05 (q, J=12.6 Hz, 2H), 3.93-3.77 (m, 1H), 3.78-3.63 (m, 2H), 3.52-3.38 (m, 2H), 3.22-3.06 (m, 4H), 2.28-2.09 (m, 2H), 2.02 (d, J=13.3 Hz, 1H), 1.74 (dd, J=16.4, 8.4 Hz, 1H), 1.48 (t, J=7.0 Hz, 3H).

### Experimental Protocol for Compound 20

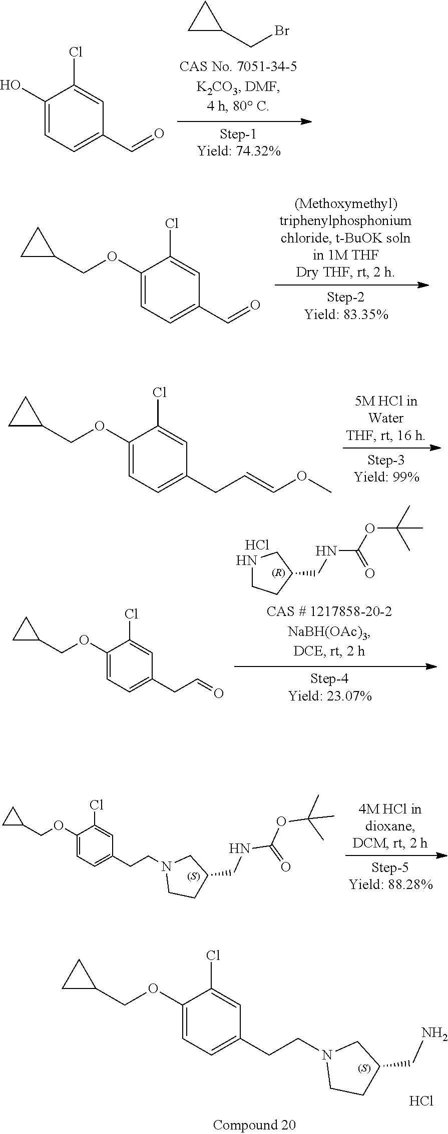

### Step-1: Synthesis of 3-chloro-4-(cyclopropylmethoxy) benzaldehyde

### Procedure

To a stirred solution of 3-chloro-4-hydroxybenzaldehyde (0.5 g, 3.19 mmol) in DMF (5 mL, 10 V) at room temperature, K₂CO₃ (1.32 g, 9.58 mmol) was added. The reaction mixture was stirred at room temperature for 1 h. Then (bromomethyl)cyclopropane (0.51 g, 3.83 mmol) was added to the reaction mixture. The reaction mixture was stirred at 80° C. for 4 h. The progress of the reaction was monitored by TLC. After completion of the reaction, the reaction mixture was poured into ice-cold water (100 mL) and extracted with ethyl acetate (3×100 mL). The combined organic fractions were washed with cold water (300 mL), dried over sodium sulphate and concentrated under reduced pressure to provide 3-chloro-4-(cyclopropylmethoxy) benzaldehyde (0.5 g, 74.32% yield) as a white solid.

¹H NMR (400 MHz, d₆-DMSO) δ: 9.85 (s, 1H), 7.95 (s, 1H), 7.88-7.85 (dd, J=8.9, 2.5 Hz, 1H), 7.34-7.32 (dd, J=8.9 Hz, 1H), 4.06 (d, J=7.0 Hz, 2H), 1.30-1.27 (s, 1H), 0.63-0.59 (d, J=8.9, 2.5 Hz, 2H) 0.40-0.36 (d, J=8.9, 2.5 Hz, 2H).

### Step-2: Synthesis of (E)-2-chloro-1-(cyclopropylmethoxy)-4-(3-methoxyallyl) benzene

### Procedure

To a stirred solution of (Methoxymethyl)triphenyl phosphine (1.01 g, 2.96 mmol) in dry THF (10 mL, 10V) at room temperature, t-BuOK in 1M THF (1.25 mL, 1.25 mmol) was added. The reaction mixture was stirred at room temperature for 1 h. 3-chloro-4-(cyclopropylmethoxy) benzaldehyde (0.25 g, 1.18 mmol) in dry THF (5 mL) was added drop wise into the reaction mixture at room temperature. The reaction mixture was stirred at room temperature for 2 h. The progress of the reaction was monitored by TLC. After completion of the reaction, the reaction mixture was concentrated under reduced pressure and the crude material purified by flash column chromatography (Aluminum oxide neutral; 0-2% ethyl acetate in hexane) to provide (E)-2-chloro-1-(cyclopropylmethoxy)-4-(3-methoxyallyl) benzene (0.25 g, 83.35%) as a colorless oil.

### Step-3: Synthesis of 2-(3-chloro-4-(cyclopropylmethoxy) phenyl)acetaldehyde

### Procedure

To a stirred solution of (E)-2-chloro-1-(cyclopropylmethoxy)-4-(3-methoxyallyl) benzene (0.25 g, 0.98 mmol) in THF (2.5 mL, 10V) at 0° C., 5M HCl (1.25 mL, 5V) was added. The reaction mixture was stirred at room temperature for 16 h. The progress of the reaction was monitored by TLC. After completion of reaction, the reaction mixture was quenched with a saturated solution of sodium bicarbonate (50 mL) and extracted with ethyl acetate (2×100 mL). The combined organic fractions were dried over sodium sulphate and concentrated under reduced pressure to provide 2-(3-chloro-4-(cyclopropylmethoxy) phenyl) acetaldehyde (0.22 g, 99%) as a light yellow liquid (confirmed using TLC analysis with a 2,4-DNP stain).

### Step-4: Synthesis of tert-butyl (S)-((1-(3-chloro-4-(cyclopropylmethoxy) phenethyl) pyrrolidin-3-yl) methyl) carbamate

### Procedure

To a stirred solution of 2-(3-chloro-4-(cyclopropylmethoxy) phenyl) acetaldehyde (0.25 g, 1.11 mmol) in DCE (2.5 mL, 10V) at room temperature, tert-butyl (R)-(pyrrolidin-3-ylmethyl) carbamate hydrochloride (0.26 g, 1.11 mmol) was added. The reaction mixture was stirred at room temperature for 1 h. Sodium triacetoxyborohydride (0.70 g, 3.33 mmol) was added to the reaction mixture in portions at 0° C. The reaction was stirred at room temperature for 1 h. The progress of the reaction was monitored by TLC and LCMS analysis. After completion of the reaction, the reaction mixture was poured into a saturated solution of sodium bicarbonate (30 mL) and extracted with CH₂Cl₂ (3×15 mL). The combined organic fractions were washed with water (30 mL), dried over sodium sulphate and concentrated under reduced pressure. The crude product material was purified by flash column chromatography (50-55% ethyl acetate in hexane] to provide tert-butyl (S)-((1-(3-chloro-4-(cyclopropylmethoxy) phenethyl) pyrrolidin-3-yl) methyl) carbamate (0.105 g, 23.07% yield) as a sticky yellow solid.

LCMS [ESI, M, M+2]: 409.2, 411.0 (RT: 1.683 min, Purity: 86.10%)

### Step-5: Synthesis of (S)-(1-(3-chloro-4-(cyclopropylmethoxy)phenethyl)pyrrolidin-3-yl)methanamine hydrochloride (Compound 20)

### Procedure

To a stirred solution of tert-butyl (S)-((1-(3-chloro-4-(cyclopropylmethoxy)phenethyl)pyrrolidin-3-yl)methyl)carbamate (0.105 g, 0.25 mmol) in CH₂Cl₂ (1.05 mL, 10V) at 0° C., 4M HCl in dioxane (0.52 mL, 5V) was added. The reaction mixture was stirred at room temperature for 2 h. The progress of the reaction was monitored by TLC and LCMS analysis. After completion of the reaction, the reaction mixture was concentrated under reduced pressure and the crude material. purified by trituration using diethyl ether (2×5 mL) and n-pentane (2×5 mL) to provide (S)-(1-(3-chloro-4-(cyclopropylmethoxy)phenethyl)pyrrolidin-3-yl)methanamine hydrochloride (0.07 g, 88.28%) as a sticky yellow solid.

LCMS [ESI, M, M+2]: 308.9, 310.7 (RT: 1.029 min, Purity: 95.89%)

HPLC: RT: 4.43 min, Purity: 95.27%

Chiral HPLC: RT: 2.91 min, Purity: 100.00%

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% METHANOLIC AMMONIA in METHANOL-ACETONITRILE (50-50) with 55-45 composition method; with Flow rate=3 ml/min; Column oven temperature 40° C.; ABPR 130 bar, analysis time 6 min.

¹H NMR (400 MHz, CD₃OD) δ: 7.38 (d, J=1.2 Hz, 1H), 7.21 (d, J=8.2 Hz, 1H), 7.03 (d, J=8.4 Hz, 1H), 3.91 (d, J=6.7 Hz, 2H), 3.74 (d, J=33.6 Hz, 1H), 3.47 (m, 4H), 3.16 (m, 2H), 3.04 (d, J=7.8 Hz, 3H), 2.83 (d, J=52.0 Hz, 1H), 2.40 (d, J=42.0 Hz, 1H), 2.09-1.83 (m, 1H), 1.30 (d, J=4.8 Hz, 1H), 0.64 (q, J=5.3 Hz, 2H), 0.40 (d, J=4.8 Hz, 2H).

### Experimental Protocol for Compound 21

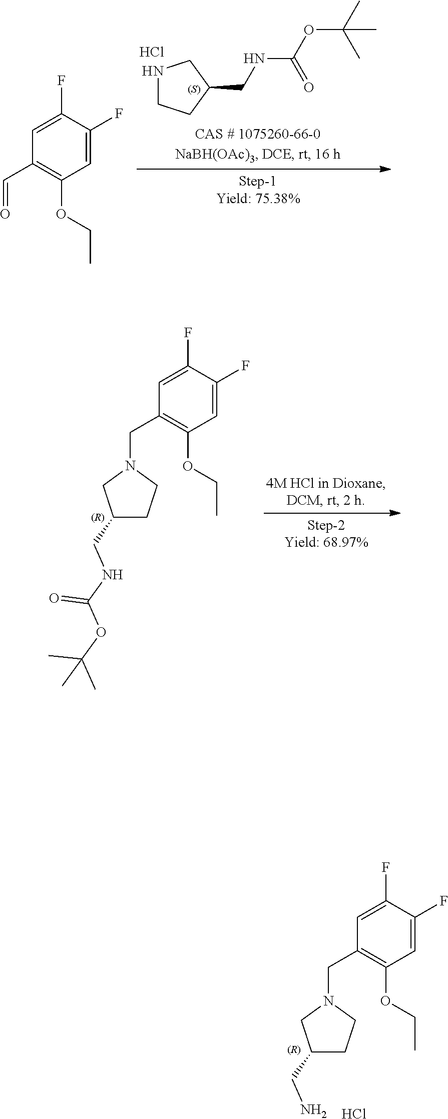

### Step-1: Synthesis of tert-butyl (R)-((1-(2-ethoxy-4,5-difluorobenzyl)pyrrolidin-3-yl)methyl)carbamate

### Procedure

To a stirred solution of 2-ethoxy-4,5-difluorobenzaldehyde (0.2 g, 1.07 mmol) in DCE (4 mL) at room temperature, tert-butyl (S)-(pyrrolidin-3-ylmethyl)carbamate hydrochloride (0.305 g, 1.28 mmol) was added. The reaction mixture was stirred at room temperature for 1 h. Sodium triacetoxyborohydride (0.683 g, 3.22 mmol) was added portion-wise to the reaction mixture at 0° C. The reaction mixture was allowed to stir at room temperature for 16 h. The progress of the reaction was monitored by TLC and LCMS analysis. After completion of the reaction, the reaction mixture was poured into water (30 mL) and extracted with CH₂Cl₂ (3×10 mL). The combined organic fractions were washed with sat.

NaHCO₃ solution (2×20 mL), dried over anhydrous sodium sulphate and concentrated under reduced pressure. The crude material was purified by flash column chromatography (neutral alumina; 0-10% CH₂Cl₂/MeOH) to provide tert-butyl (R)-((1-(2-ethoxy-4,5-difluorobenzyl)pyrrolidin-3-yl)methyl)carbamate (0.3 g, 75.38% yield) as a yellow oil.

LCMS [ESI, M+1]: 371.2 (RT: 1.432 min, Purity: 97.55%),

Chiral HPLC: RT: 10.21 min, Purity: 97.96%

¹H NMR (400 MHz, d₆-DMSO): δ 7.29 (dd, J=26.4, 16.0 Hz, 1H), 7.11 (dd, J=12.9, 6.9 Hz, 1H), 6.91 (s, 1H), 4.05 (dq, J=13.9, 7.1 Hz, 2H), 3.59-3.46 (m, 2H), 2.92 (t, J=6.0 Hz, 2H), 2.46 (dd, J=19.7, 11.7 Hz, 3H), 2.29 (dd, J=8.8, 5.2 Hz, 1H), 2.23 (s, 1H), 1.91-1.75 (m, 1H), 1.38 (d, J=11.9 Hz, 9H), 1.34 (t, J=6.9 Hz, 3H).

### Step-2: Synthesis of (R)-(1-(2-ethoxy-4,5-difluorobenzyl)pyrrolidin-3-yl)methanamine hydrochloride (Compound 21)

### Procedure

To a stirred solution of tert-butyl (R)-((1-(2-ethoxy-4,5-difluorobenzyl)pyrrolidin-3-yl)methyl)carbamate (0.3 g, 0.810 mmol) in CH₂Cl₂ (3.0 mL) at 0° C., 4M HCl in Dioxane (1.5 mL, 5V) was added. The reaction mixture was stirred at room temperature for 2 h. The progress of the reaction was monitored by TLC and LCMS analysis. After completion of the reaction, the reaction mixture was concentrated under reduced pressure and the crude material triturated with diethyl ether:CH₂Cl₂ (9:1) (20 mL) to provide (R)-(1-(2-ethoxy-4,5-difluorobenzyl)pyrrolidin-3-yl)methanamine hydrochloride (0.15 g, 68.97% yield,) as an off-white sticky solid.

LCMS [ESI, M+1]: 271.0 (RT: 0.698 min, Purity: 98.40%),

HPLC: RT: 5.75 min, Purity: 96.12%,

Chiral HPLC: RT: 5.804 min, Purity: 99.79%,

Chromatographic Conditions:

Column: CHIRALPAK® IA (250×4.6 mm), 5.0 μm,

Mobile phase: n-Hexane/MTBE/MeOH/EDA (60/35/05/0.1%),

Elution mode: Isocratic

Flow rate: 1.0 mL/min, COT: 25° C., UV: 280 nm,

Sample conc: 1.0 mg/mL, Diluent:MeOH/Mobilephase (50/50).

¹H NMR (400 MHz, CD₃OD): δ 7.52 (d, J=9.2 Hz, 1H), 7.13 (dd, J=12.4, 6.7 Hz, 1H), 4.47-4.33 (m, 2H), 4.25-4.07 (q, J=12.4 Hz, 2H), 3.72 (dd, J=24.0, 17.8 Hz, 1H), 3.73-3.49 (m, 2H), 3.41 (s, 1H), 3.11 (dd, J=25.3, 8.0 Hz, 2H), 2.82 (d, J=63.9 Hz, 1H), 2.38 (d, J=49.6 Hz, 1H), 1.94 (d, J=70.9 Hz, 1H), 1.47 (t, J=6.9 Hz, 3H).

### Experimental Protocol for Compound 22

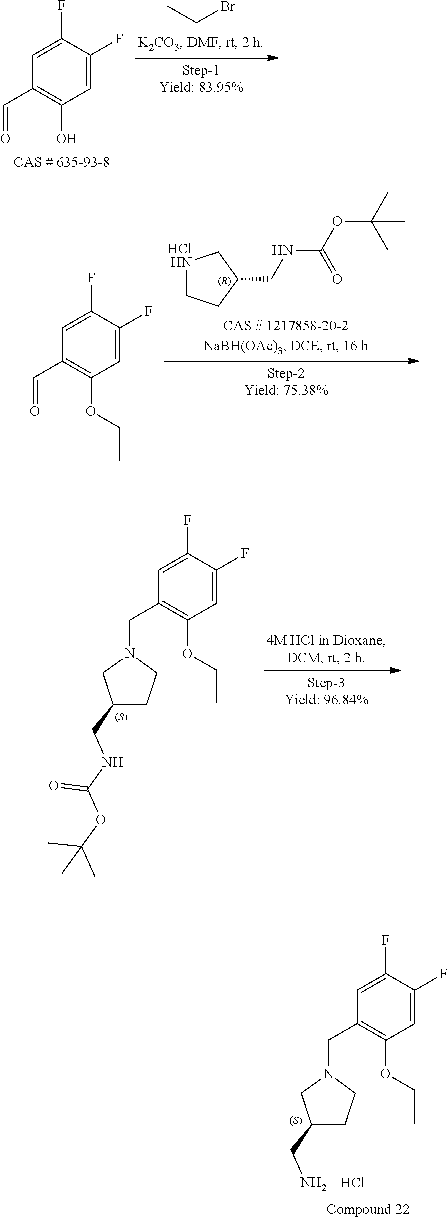

### Step-1: Synthesis of 2-ethoxy-4,5-difluorobenzaldehyde

### Procedure

To a stirred solution of 4,5-difluoro-2-hydroxybenzaldehyde (1.0 g, 6.32 mmol) in DMF (10 mL) at room temperature, potassium carbonate (2.6 g, 18.97 mmol) was added. The reaction mixture was stirred at room temperature for 30 min. Ethyl bromide (0.703 g, 6.96 mmol) was then added to the reaction mixture. The reaction mixture was stirred at room temperature for 1.5 h. The progress of the reaction was monitored by TLC. After completion of the reaction, the reaction mixture was poured into cold water (100 mL). A solid precipitate was formed which was isolated by filtration and dried under reduced pressure to provide 2-ethoxy-4,5-difluorobenzaldehyde (1.02, 83.95% yield) as a white solid.

LCMS [ESI, M+1]: 186.9 (RT: 1.902 min, Purity: 100%), ¹H NMR (400 MHz, d₆-DMSO): δ 10.25 (d, J=3.1 Hz, 1H), 7.65 (t, J=9.9 Hz, 1H), 7.46 (dd, J=12.8, 6.4 Hz, 1H), 4.19 (q, J=7.0 Hz, 2H), 1.38 (t, J=7.0 Hz, 3H).

### Step-2: Synthesis of tert-butyl (S)-((1-(2-ethoxy-4,5-difluorobenzyl)pyrrolidin-3-yl)methyl)carbamate

### Procedure

To a stirred solution of 2-ethoxy-4,5-difluorobenzaldehyde (0.2 g, 1.07 mmol) in DCE (4 mL) at room temperature, tert-butyl (R)-(pyrrolidin-3-ylmethyl)carbamate hydrochloride (0.305 g, 1.28 mmol) was added. The reaction mixture was stirred at room temperature for 1 h. Then, sodium triacetoxyborohydride (0.683 g, 3.22 mmol) was added portion-wise to the reaction mixture at 0° C. The reaction mixture was allowed to stir at room temperature for 16 h. The progress of the reaction was monitored by TLC and LCMS analysis. After completion of the reaction, the reaction mixture was poured into water (30 mL) and extracted with CH₂Cl₂ (3×20 mL). The combined organic fractions were washed with sat. NaHCO₃ solution (2×20 mL), dried over anhydrous sodium sulphate and concentrated under reduced pressure. The crude material was purified by flash column chromatography (neutral alumina; 0-10% MeOH in CH₂Cl₂) to provide tert-butyl (S)-((1-(2-ethoxy-4,5-difluorobenzyl)pyrrolidin-3-yl)methyl)carbamate (0.3 g, 75.38% yield) as a yellow oil.

LCMS [ESI, M+1]: 371.2 (RT: 1.509 min, Purity: 100%),

Chiral HPLC: RT: 9.71 min, Purity: 96.69%

¹H NMR (400 MHz, d₆-DMSO): δ 7.29 (dd, J=26.4, 16.0 Hz, 1H), 7.11 (dd, J=12.9, 6.9 Hz, 1H), 6.91 (s, 1H), 4.05 (dq, J=13.9, 7.1 Hz, 2H), 3.59-3.46 (m, 2H), 2.92 (t, J=6.0 Hz, 2H), 2.46 (dd, J=19.7, 11.7 Hz, 3H), 2.29 (dd, J=8.8, 5.2 Hz, 1H), 2.23 (s, 1H), 1.91-1.75 (m, 1H), 1.38 (d, J=11.9 Hz, 9H), 1.34 (t, J=6.9 Hz, 3H).

### Step-3: Synthesis of (S)-(1-(2-ethoxy-4,5-difluorobenzyl)pyrrolidin-3-yl)methanamine hydrochloride (Compound 22)

### Procedure

To a stirred solution of tert-butyl (S)-((1-(2-ethoxy-4,5-difluorobenzyl)pyrrolidin-3-yl)methyl)carbamate (0.15 g, 0.347 mmol) in CH₂Cl₂ (1.5 mL) at 0° C., 4M HCl in Dioxane (0.75 mL, 5V) was added. The reaction mixture was stirred at room temperature for 2 h.

The progress of the reaction was monitored by TLC and LCMS analysis. After completion of the reaction, the reaction mixture was concentrated under reduced pressure. The resultant crude material was triturated with diethyl ether:CH₂Cl₂ (9:1) (2×15 mL) to provide (S)-(1-(2-ethoxy-4,5-difluorobenzyl)pyrrolidin-3-yl)methanamine hydrochloride (0.12, 96.84% yield) as an off-white sticky solid.

LCMS [ESI, M+1]: 270.9 (RT: 0.754 min, Purity: 96.98%),

HPLC: RT: 3.95 min, Purity: 97.51%,

Chiral HPLC: RT: 7.104 min, Purity: 99.74%,

Chromatographic Conditions:

Column: CHIRALPAK® IA (250×4.6 mm), 5.0 μm,

Mobile phase: n-Hexane/MTBE/MeOH/EDA (60/35/05/0.1%),

Elution mode: Isocratic

Flow rate: 1.0 mL/min, COT: 25° C., UV: 280 nm,

Sample conc: 1.0 mg/mL, Diluent:MeOH/Mobilephase (50/50).

¹H NMR (400 MHz, CD₃OD): δ 7.54 (dd, J=19.1, 9.6 Hz, 1H), 7.15 (dd, J=12.4, 6.7 Hz, 1H), 4.51-4.37 (m, 2H), 4.26-4.11 (q, J=12.4 Hz, 2H), 3.83-3.73 (m, 1H), 3.71-3.58 (m, 2H), 3.44 (s, 1H), 3.13 (dd, J=25.2, 8.1 Hz, 2H), 2.99-2.72 (m, 1H), 2.53-2.27 (m, 1H), 1.96 (d, J=65.7 Hz, 1H), 1.50 (t, J=6.9 Hz, 3H).

### Experimental Protocol for Compound 23

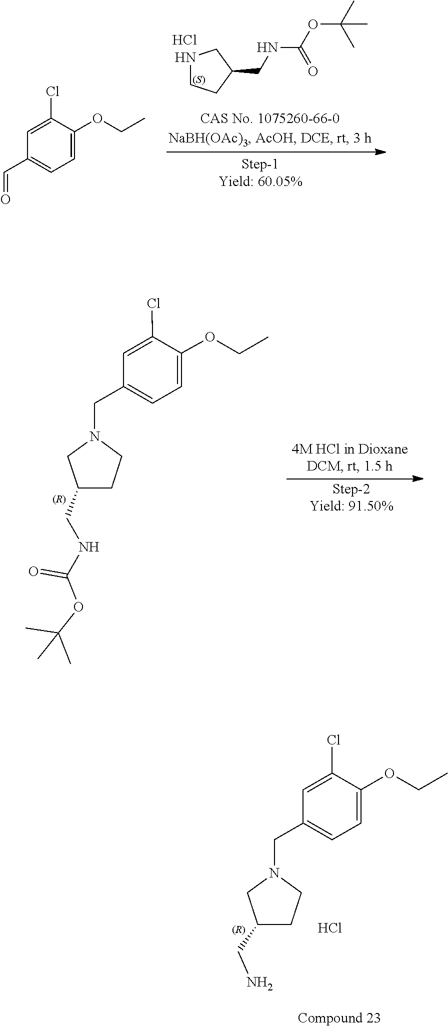

### Step-1: Synthesis of tert-butyl (R)-((1-(3-chloro-4-ethoxybenzyl) pyrrolidin-3-yl)methyl)carbamate

### Procedure

To a stirred solution of 3-chloro-4-ethoxybenzaldehyde (2.5 g, 13.54 mmol) in DCE (25.0 mL, 10V) at room temperature, tert-butyl (S)-(pyrrolidin-3-ylmethyl) carbamate hydrochloride (3.84 g, 16.24 mmol) and Acetic acid (0.125 mL, 0.05V) were added. The reaction mixture was stirred at room temperature for 2 h. Sodium triacetoxyborohydride (8.60 g, 40.62 mmol) was added to the reaction mixture portion-wise at 0° C. The reaction was stirred at room temperature for 1 h. The progress of the reaction was monitored by TLC and LCMS analysis. After completion of the reaction, the reaction mixture was poured into a saturated solution of sodium bicarbonate (100 mL) and extracted with CH₂Cl₂ (3×70 mL). The combined organic fractions were washed with water (100 mL), dried over sodium sulphate and concentrated under reduced pressure. The crude material was purified by column chromatography (SiO₂; 45-50% EtOAc in hexane) to provide tert-butyl (R)-((1-(3-chloro-4-ethoxybenzyl)pyrrolidin-3-yl)methyl)carbamate (3.0 g, 60.05% yield) as a yellow sticky solid.

LCMS [ESI, M, M+2]: 369.0, 370.7 (RT: 1.414 min, Purity: 80.53%)

### Step-2: Synthesis of (R)-(1-(3-chloro-4-ethoxybenzyl)pyrrolidin-3-yl) methanamine hydro chloride (Compound 23)

### Procedure

To a stirred solution of tert-butyl (R)-((1-(3-chloro-4-ethoxybenzyl) pyrrolidin-3-yl) methyl) carbamate (3.0 g, 8.13 mmol) in CH₂Cl₂ (30 mL, 10V) at 0° C., 4M HCl in dioxane (15 mL, 5V) was added. The reaction mixture was stirred at room temperature for 1.5 h.

The progress of the reaction was monitored by TLC and LCMS analysis. After completion of the reaction, the reaction mixture was concentrated under reduced pressure and the resultant crude material purified by trituration with CH₂Cl₂ (2×30 mL) to afford (R)-(1-(3-chloro-4-ethoxybenzyl)pyrrolidin-3-yl) methanamine hydrochloride (2.02, 91.50%) as a white solid.

LCMS [ESI, M&M+2]: 268.8, 270.7 (RT: 0.776 min, Purity: 96.39%)

HPLC: RT: 4.07 min, Purity: 100.00%

Chiral HPLC: RT: 8.646 min, Purity: 100.00%

Instrument Name: Waters SFC Investigator

Chromatographic separation was conducted with Waters SFC Investigator system with 2998 PDA detector. The column used was CHIRALPAK® IH (250×4.6 mm; 5 ␣m) and the compounds were eluted with, Mobile Phase A: Liq·CO2, Mobile Phase B: 0.1% METHANOLIC AMMONIA in 2-PROPANOL-MTBE (70-30) with 80-20 composition method; with Flow rate=3 ml/min; Column oven temperature 40° C.; ABPR 130 bar, analysis time 20 min.

¹H NMR (400 MHz, D₂O) δ 7.44 (s, 1H), 7.27 (d, J=8.4 Hz, 1H), 7.06 (d, J=8.5 Hz, 1H), 4.27-4.17 (m, 2H), 4.10 (q, J=6.9 Hz, 2H), 3.50 (s, 1H), 3.33 (s, 2H), 3.00 (p, J=12.9 Hz, 3H), 2.66 (s, 1H), 2.24 (s, 1H), 1.75 (s, 1H), 1.29 (t, J=7.0 Hz, 3H).

### Experimental Protocol for Compound 24

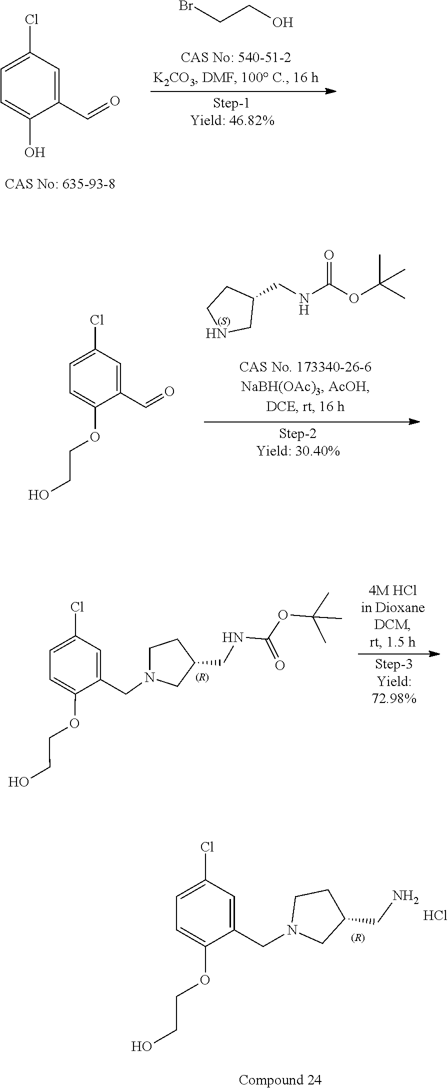

### Step-1: Synthesis of 5-chloro-2-(2-hydroxyethoxy)benzaldehyde

### Procedure

To a stirred solution of 5-chloro-2-hydroxybenzaldehyde (1.0 g, 6.386 mmol) in DMF (10 mL) at room temperature, K₂CO₃ (2.6 g, 1.916 mmol) was added. The reaction mixture was stirred at room temperature for 1 h. 2-bromoethan-1-ol (1.18 g, 9.580 mmol) was added to the reaction mixture and the reaction mixture was stirred at 100° C. for 16 h. The progress of the reaction was monitored by TLC. After completion of the reaction, the reaction mixture was poured into ice-cold water (50 mL) and extracted with ethyl acetate (3×40 mL). The combined organic fractions were dried over sodium sulphate and concentrated under reduced pressure. The crude material was purified by flash column chromatography (SiO₂; 37% ethyl acetate in hexane) to provide 5-chloro-2-(2-hydroxyethoxy)benzaldehyde (0.6 g, 46.82% yield) as a viscous yellow liquid.

¹H NMR (400 MHz, d₆-DMSO) δ 10.39 (s, 1H), 7.69 (dd, J=8.9, 2.7 Hz, 1H), 7.61 (d, J=2.7 Hz, 1H), 7.29 (t, J=9.5 Hz, 1H), 5.00 (s, 1H), 4.15 (dd, J=17.4, 12.7 Hz, 2H), 3.77 (t, J=4.2 Hz, 2H).

### Step-2: Synthesis of tert-butyl(R)-((1-(5-chloro-2-(2-hydroxyethoxy)benzyl)pyrrolidin-3-yl)methyl) carbamate

### Procedure

To a stirred solution of 5-chloro-2-(2-hydroxyethoxy)benzaldehyde (0.6 g, 3.0 mmol) in DCE (6.0 mL) at room temperature, tert-butyl (S)-(pyrrolidin-3-ylmethyl)carbamate (0.849 g, 3.6 mmol) and Acetic Acid (0.01 mL, 0.05V) were added. The reaction mixture was stirred at room temperature for 4 h. Then sodium triacetoxyborohydride (1.89 g, 9.0 mmol) was added to the reaction mixture portion-wise at 0° C. The reaction was stirred at room temperature for 12 h. The progress of the reaction was monitored by TLC and LCMS analysis. After completion of the reaction, the reaction mixture was poured into a saturated solution of sodium bicarbonate (40 mL) and extracted with CH₂Cl₂ (3×30 mL). The combined organic fractions were washed with water (30 mL), dried over sodium sulphate and concentrated under reduced pressure. The crude material was purified by flash column chromatography (neutral SiO₂; 10% ethyl acetate in hexane) to provide tert-butyl (R)-((1-(5-chloro-2-(2-hydroxyethoxy)benzyl)pyrrolidin-3-yl)methyl)carbamate (0.35 g, 30.40% yield) as a yellow liquid.

LCMS [ESI, M+1]: 385.0 (RT: 1.409 min, Purity: 93.48%)

### Step-3: Synthesis of (R)-2-(2-((3-(aminomethyl)pyrrolidin-1-yl)methyl)-4-chlorophenoxy)ethan-1-ol hydrochloride ((Compound 24)

### Procedure

To a stirred solution of tert-butyl (R)-((1-(5-chloro-2-(2-hydroxyethoxy)benzyl)pyrrolidin-3-yl)methyl)carbamate (0.3 g, 0.781 mmol) in CH₂Cl₂ (3.0 mL, 10V) at 0° C., 4M HCl in Dioxane (1.5 mL, 5V) was added. The reaction mixture was stirred at room temperature for 2 h. The progress of the reaction was monitored by TLC and LCMS analysis. After completion of the reaction, the reaction mixture was concentrated under reduced pressure and the crude material purified by reverse phase column chromatography (21% CH₃CN/water) to provide (R)-2-(2-((3-(aminomethyl)pyrrolidin-1-yl)methyl)-4-chlorophenoxy)ethan-1-ol hydrochloride (0.162 g, 72.98% yield) as an off-white sticky solid.

LCMS [ESI, M+1]: 284.8 (RT: 0.660 min, Purity: 100%),

Chiral HPLC: 2.81 min, Purity: 100%

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% METHANOLIC AMMONIA in METHANOL-ACETONITRILE (50-50) with 60-40 composition method; with Flow rate=3 ml/min; Column oven temperature 40° C.; ABPR 130 bar, analysis time 6 min.

HPLC: RT: 3.82 min, Purity: 100%

¹H NMR (400 MHz, CD₃OD) δ 7.59 (dd, J=4.9, 2.3 Hz, 1H), 7.48 (dd, J=8.8, 2.0 Hz, 1H), 7.16 (d, J=8.8 Hz, 1H), 4.53 (q, J=12.9 Hz, 2H), 4.24 (d, J=3.7 Hz, 2H), 3.99 (d, J=3.8 Hz, 2H), 3.77-3.60 (m, 2H), 3.48 (dd, J=18.8, 9.6 Hz, 1H), 3.40 (dd, J=12.9, 6.4 Hz, 1H), 3.15 (dd, J=20.0, 6.9 Hz, 2H), 2.78 (dd, J=15.9, 7.9 Hz, 1H), 2.52-2.29 (m, 1H), 2.12-1.77 (m, 1H).

### Experimental Protocol for Compound 25

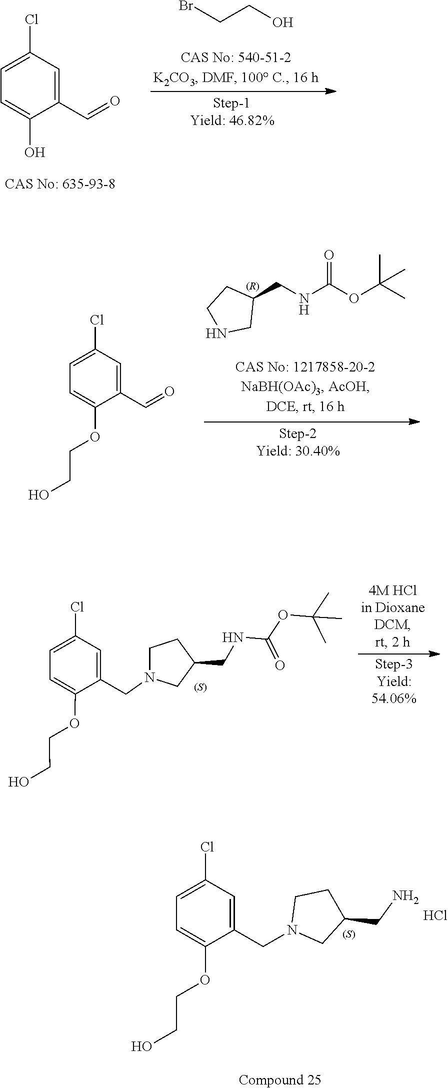

### Step1: Synthesis of 5-chloro-2-(2-hydroxyethoxy)benzaldehyde

### Procedure

To a stirred solution of 5-chloro-2-hydroxybenzaldehyde (1.0 g, 6.386 mmol) in DMF (10 mL) at room temperature, K₂CO₃ (2.6 g, 1.916 mmol) was added. The reaction mixture was stirred at room temperature for 1 h. Then 2-bromoethan-1-ol (1.18 g, 9.580 mmol) was added to the reaction mixture and the reaction mixture was stirred at 100° C. for 16 h. The progress of the reaction was monitored by TLC. After completion of the reaction, the reaction mixture was poured into ice-cold water (50 mL) and extracted with ethyl acetate (3×40 ml). The combined organic fractions were dried over sodium sulphate and concentrated under reduced pressure. The crude material was purified by flash column chromatography (SiO₂; 37% ethyl acetate in hexane) to provide 5-chloro-2-(2-hydroxyethoxy)benzaldehyde (0.6 g, 46.82% yield) as a yellow sticky liquid.

¹H NMR (400 MHz, d₆-DMSO) δ 10.39 (s, 1H), 7.69 (dd, J=8.9, 2.7 Hz, 1H), 7.61 (d, J=2.7 Hz, 1H), 7.29 (t, J=9.5 Hz, 1H), 5.00 (s, 1H), 4.15 (dd, J=17.4, 12.7 Hz, 2H), 3.77 (t, J=4.2 Hz, 2H).

### Step2: Synthesis of tert-butyl (S)-((1-(5-chloro-2-(2-hydroxyethoxy)benzyl)pyrrolidin-3-yl)methyl) carbamate

### Procedure

To a stirred solution of 5-chloro-2-(2-hydroxyethoxy)benzaldehyde (0.6 g, 3.0 mmol) in DCE (6.0 mL) at room temperature, tert-butyl (R)-(pyrrolidin-3-ylmethyl)carbamate hydrochloride (0.849 g, 3.6 mmol) and Acetic Acid (0.01 mL, 0.05V) were added. The reaction mixture was stirred at room temperature for 4 h. Then sodium triacetoxyborohydride (1.89 g, 9.0 mmol) was added to the reaction mixture portion-wise at 0° C. The reaction was stirred at room temperature for 12 h. The progress of the reaction was monitored by TLC and LCMS analysis. After completion of the reaction, the reaction mixture was poured into a saturated solution of sodium bicarbonate (40 mL) and extracted with CH₂Cl₂ (3×30 mL). The combined organic fractions were washed with water (30 mL), dried over sodium sulphate and concentrated under reduced pressure. The crude material was purified by flash column chromatography (neutral SiO₂; 10% ethyl acetate in hexane) to provide tert-butyl (S)-((1-(5-chloro-2-(2-hydroxyethoxy)benzyl)pyrrolidin-3-yl)methyl)carbamate (0.35 g, 30.40% yield) as a yellow liquid.

LCMS [ESI, M+1]: 385.0 (RT: 1.310 min, Purity: 90.03%)

### Step 3: Synthesis of (S)-2-(2-((3-(aminomethyl)pyrrolidin-1-yl)methyl)-4-chlorophenoxy)ethan-1-ol hydrochloride (Compound 25)

### Procedure

To a stirred solution of tert-butyl (S)-((1-(5-chloro-2-(2-hydroxyethoxy)benzyl)pyrrolidin-3-yl)methyl)carbamate (0.35 g, 0.911 mmol) in CH₂Cl₂ (3.5 mL, 10V) at 0° C., 4M HCl in Dioxane (1.75 mL, 5V) was added. The reaction mixture was stirred at room temperature for 2 h. The progress of the reaction was monitored by TLC and LCMS analysis. After completion of the reaction, the reaction mixture was concentrated under reduced pressure. The crude material was purified by reverse phase column chromatography (19% CH₃CN/water) to provide (S)-2-(2-((3-(aminomethyl)pyrrolidin-1-yl)methyl)-4-chlorophenoxy)ethan-1-ol hydrochloride (0.144 g, 54.06%) as an off-white sticky solid.

LCMS [ESI, M+1]: 284.8 (RT: 0.653 min, Purity: 100%),

Chiral HPLC: 2.75 min, Purity: 100%

Instrument Name: Waters SFC Investigator

Chromatographic separation was conducted with Waters SFC Investigator system with 2998 PDA detector. The column used was CHIRALPAK® IG (250×4.6 mm; 5 ␣m) and the compounds were eluted with, Mobile Phase A: Liq·CO2, Mobile Phase B: 0.1% METHANOLIC AMMONIA in METHANOL-ACETONITRILE (50-50) with 55-45 composition method; with Flow rate=3 ml/min; Column oven temperature 40° C.; ABPR 130 bar, analysis time 6 min.

HPLC: RT: 3.653 min, Purity: 100%

¹H NMR (400 MHz, CD₃OD) δ 7.60-7.54 (m, 1H), 7.50 (dd, J=8.8, 2.4 Hz, 1H), 7.16 (t, J=8.5 Hz, 1H), 4.58-4.41 (m, 2H), 4.25 (s, 2H), 4.05-3.92 (m, 2H), 3.68 (ddd, J=18.6, 16.2, 8.4 Hz, 2H), 3.50-3.37 (m, 2H), 3.20-3.08 (m, 2H), 2.99-2.69 (m, 1H), 2.54-2.29 (m, 1H), 2.11-1.79 (m, 1H).

### Experimental Protocol for Compound 26

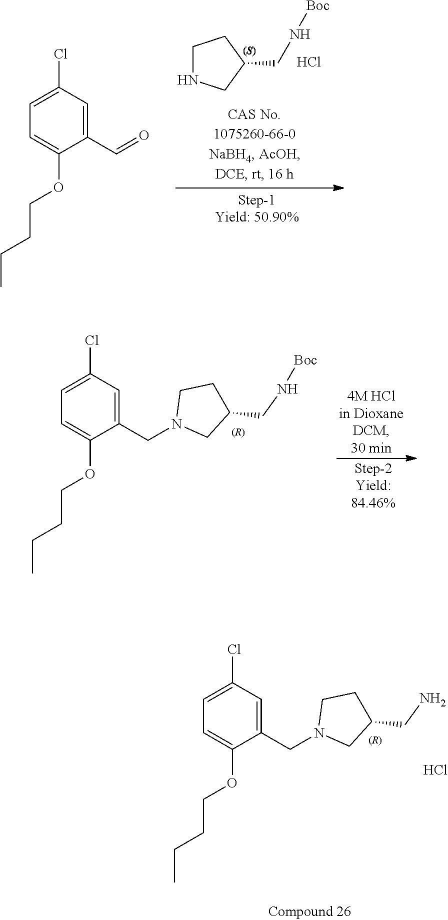

### Step-1: Synthesis of tert-butyl (R)-((1-(2-butoxy-5-chlorobenzyl)pyrrolidin-3-yl)methyl)carbamate

### Procedure

To a stirred solution of 2-butoxy-5-chlorobenzaldehyde (0.2 g, 0.94 mmol) in DCE (2 mL, 10V) at room temperature, tert-butyl (S)-(pyrrolidin-3-ylmethyl)carbamate hydrochloride (0.26 g, 1.12 mmol) followed by acetic acid (0.01 mL, 0.05V) were added. The reaction mixture was stirred at room temperature for 2 h, then sodium borohydride (0.071 g, 1.88 mmol) was added to reaction mixture at 0° C. The reaction mixture was stirred at room temperature for 16 h. The progress of the reaction was monitored by TLC and LCMS analysis. After completion of the reaction, the reaction mixture was diluted with a saturated solution of sodium bicarbonate (30 mL) and extracted with CH₂Cl₂ (3×20 mL). The combined organic fractions were dried over sodium sulfate and concentrated under reduced pressure. The crude material was purified by column chromatography (SiO₂; eluted in 58% EtOAc in hexane) to provide a tert-butyl (R)-((1-(2-butoxy-5-chlorobenzyl)pyrrolidin-3-yl)methyl)carbamate (0.192, 50.90% yield) as a pale yellow sticky liquid.

LCMS [ESI, M+1]: 397.34 (RT: 2.138 min, Purity: 97.42%),

### Step-2: Synthesis of (R)-(1-(2-butoxy-5-chlorobenzyl)pyrrolidin-3-yl)methanamine hydrochloride (Compound 26)

### Procedure

To a stirred solution of tert-butyl (R)-((1-(2-butoxy-5-chlorobenzyl)pyrrolidin-3-yl)methyl)carbamate (0.19 g, 0.47 mmol) in CH₂Cl₂ (1.9 mL, 10V) at 0° C., 4M HCl in Dioxane (0.9 mL, 5 V) was added. The reaction mixture was stirred at room temperature for 30 min. The progress of the reaction was monitored by TLC and LCMS analysis. After completion of the reaction, the reaction mixture was concentrated under reduced pressure and the resultant crude material purified by trituration using diethyl ether (2×20 mL) to provide (R)-(1-(2-butoxy-5-chlorobenzyl)pyrrolidin-3-Yl)methanamine hydrochloride (0.12 g, 84.46% yield) as an off-white solid.

LCMS [ESI, M+1]: 296.94 (RT: 1.022 min, 100% Purity)

HPLC: RT: 4.453 min, 99.79% Purity

Chiral HPLC: RT: 2.74 min, 97.67% Purity

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% METHANOLIC AMMONIA in METHANOL-ACETONITRILE (50-50) with 65-35 composition method; with Flow rate=3 ml/min; Column oven temperature 40° C.; ABPR 130 bar, analysis time 6 min.

¹H NMR (400 MHz, D₂O): δ 7.38-7.33 (m, 2H), 6.99 (d, J=8.8 Hz 1H), 4.30 (s, 2H), 4.02 (t, J=8 Hz, 4 Hz 2H), 3.64-3.52 (m, 2H), 3.23 (s, 2H), 3.06-3.04 (m, 3H), 2.62 (s, 1H), 2.33 (s, 1H), 1.70-1.65 (m, 2H), 1.38-1.30 (m, 2H), 0.83 (t, J=8, 4 Hz, 3H).

### Experimental Protocol for Compound 27

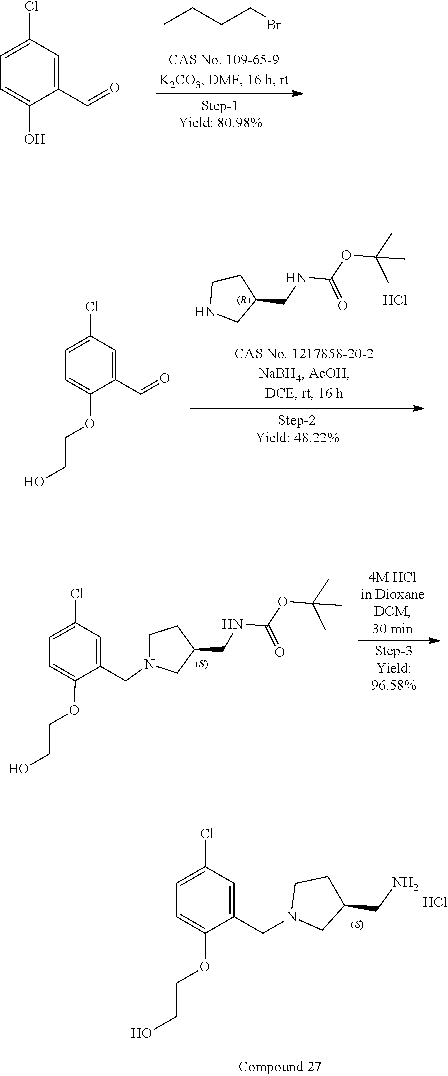

### Step-1: Synthesis of 2-butoxy-5-chlorobenzaldehyde

### Procedure

To a stirred solution of 5-chloro-2-hydroxybenzaldehyde (1.0 g, 6.38 mmol) in DMF (10 mL, 10V) at room temperature, potassium carbonate (2.64 g, 19.14 mmol) was added. The reaction mixture was stirred at room temperature for 30 min. Then 1-bromobutane (1.04 g, 7.65 mmol) was added at 0° C. The reaction mixture was then stirred at room temperature for 16 h. The progress of the reaction was monitored by TLC and LCMS analysis. After completion of the reaction, the reaction mixture was poured into ice cold water (100 mL) and extracted with EtOAc (3×50 mL). The combined organic fractions were washed with brine solution (3×50 mL), dried over sodium sulphate and concentrated under reduced pressure to provide 2-butoxy-5-chlorobenzaldehyde (1.12, 80.98% yield) as a white liquid.

¹H NMR (400 MHz, d₆-DMSO): δ 10.39 (s, 1H), 7.68 (dd, J=2.8, 1.2 Hz 1H), 7.61 (d, J=4 Hz, 1H), 7.28 (d, J=12 Hz, 1H), 4.14 (t, 2H), 1.79-1.72 (m, 2H), 1.49-1.43 (m, 2H), 0.96 (t, 3H).

### Step-2: Synthesis of tert-butyl (S)-((1-(2-butoxy-5-chlorobenzyl)pyrrolidin-3-yl)methyl)carbamate

### Procedure

To a stirred solution of 2-butoxy-5-chlorobenzaldehyde (0.2 g, 0.94 mmol) and tert-butyl (R)-(pyrrolidin-3-ylmethyl) carbamate hydrochloride (0.26 g, 1.13 mmol) in DCE (2 mL, 10V) at room temperature, acetic acid (0.01 mL, 0.05V) was added. The reaction mixture was stirred at room temperature for 2 h. Then sodium borohydride (0.071 g, 1.88 mmol) was added to reaction mixture at 0° C. The reaction mixture was stirred at room temperature for 16 h. The progress of the reaction was monitored by TLC and LCMS analysis. After completion of the reaction, the reaction mixture was poured into a saturated solution of sodium bicarbonate (30 mL) and extracted with CH₂Cl₂ (3×30 mL). The combined organic fractions were dried over sodium sulfate and concentrated under reduced pressure. The crude material was purified by column chromatography (SiO₂; 46% ethyl acetate in hexane) to provide tert-butyl (S)-((1-(2-butoxy-5-chlorobenzyl) pyrrolidin-3-yl) methyl) carbamate (0.182, 48.22% yield) as a pale yellow sticky liquid.

LCMS [ESI, M+1]: 397.22 (RT: 2.070 min, Purity: 98.94%).

### Step-3: Synthesis of (S)-(1-(2-butoxy-5-chlorobenzyl)pyrrolidin-3-yl)methanamine hydrochloride (Compound 27)

### Procedure

To a stirred solution of tert-butyl (S)-((1-(2-butoxy-5-chlorobenzyl)pyrrolidin-3-yl)methyl)carbamate (0.18 g, 0.45 mmol) in CH₂Cl₂ (1.8 mL, 10V) at 0° C., 4M HCl in Dioxane (0.9 mL, 5 V) was added. The reaction mixture was stirred at room temperature for 30 min. The progress of the reaction was monitored by TLC and LCMS analysis. After completion of reaction, the reaction mixture was concentrated under reduced pressure and the resultant crude material purified by trituration using diethyl ether (3×10 mL) to provide (S)-(1-(2-butoxy-5-chlorobenzyl)pyrrolidin-3-yl)methanamine hydrochloride (0.132, 96.58% yield) as an off-white solid.

LCMS [ESI, M+1]: 296.89 (RT: 1.013 min, 100% Purity)

HPLC: RT: 4.453 min, 99.88% Purity

Chiral HPLC: RT: 2.99 min, 100% Purity

Instrument Name: Waters SFC Investigator

Chromatographic separation was conducted with Waters SFC Investigator system with 2998 PDA detector. The column used was YMC CELLULOSE SC (250×4.6 mm; 5 ␣m) and the compounds were eluted with, Mobile Phase A: Liq·CO2, Mobile Phase B: 0.1% METHANOLIC AMMONIA in METHANOL-ACETONITRILE (50-50) with 55-45 composition method; with Flow rate=3 ml/min; Column oven temperature 40° C.; ABPR 130 bar, analysis time 7 min.

¹H NMR (400 MHz, d₆-DMSO): δ 8.13 (d, J=24, 2H), 7.71 (t, J=4 Hz, 2.8 Hz 1H), 7.46 (d, J=8 Hz 1H), 7.14 (d, J=8 Hz, 1H), 4.30-4.26 (m, 2H), 4.04 (t, J=8 Hz, 4 Hz, 2H), 3.57-3.43 (m, 2H), 3.27-3.22 (m, 2H), 2.92-2.89 (m, 2H), 2.68-2.67 (m, 1H), 2.33-2.10 (m, 1H), 1.80-1.71 (m, 3H), 1.50-1.40 (m, 2H), 0.95 (t, J=8 Hz, J=8 Hz 3H).

### Experimental Protocol for Compound 28

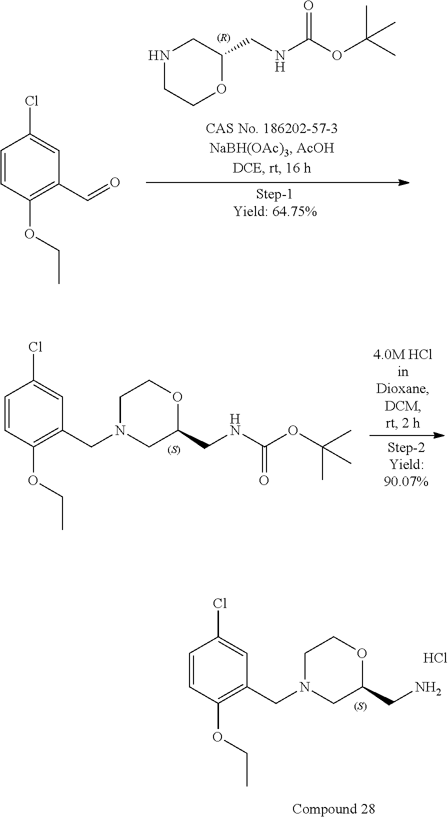

### Step-1: Synthesis of tert-butyl (S)-((4-(5-chloro-2-ethoxybenzyl)morpholin-2-yl)methyl)carbamate

### Procedure

To a stirred solution of 5-chloro-2-ethoxybenzaldehyde (0.2 g, 1.8 mmol) in DCE (4.0 mL, 20V) at room temperature, tert-butyl (R)-(morpholin-2-ylmethyl) carbamate (0.281 g, 1.2 mmol) was added. The reaction mixture was stirred at room temperature for 2 h. Sodium triacetoxyborohydride (0.688, 0.32 mmol) was added portion-wise to the reaction mixture at 0° C. Then the reaction was stirred at room temperature for 16 h. The progress of the reaction was monitored by TLC and LCMS analysis. After completion of the reaction, the reaction mixture was poured into a saturated solution of sodium bicarbonate (20 mL) and extracted with CH₂Cl₂ (3×20 mL). The combined organic fractions were washed with water (20 mL), dried over sodium sulphate and concentrated under reduced pressure. The crude material was purified by column chromatography (SiO₂; 27% ethyl acetate in hexane) to provide tert-butyl (S)-((4-(5-chloro-2-ethoxybenzyl)morpholin-2-yl)methyl)carbamate (0.27 g, 64.75% yield) as a light yellow liquid.

LCMS [ESI, M+1]: 384.91 (RT: 1.415 min, Purity: 99.48%),

### Step-2: Synthesis of (S)-(4-(5-chloro-2-ethoxybenzyl)morpholin-2-yl)methanamine hydrochloride (Compound 28)

### Procedure

To a stirred solution of tert-butyl (S)-((4-(5-chloro-2-ethoxybenzyl)morpholin-2-yl)methyl)carbamate (0.27 g, 0.7 mmol) in CH₂Cl₂ (3 mL, 10V) at 0° C., 4M HCl in dioxane (1.3 mL, 5V) was added. The reaction mixture was stirred at room temperature for 2 h. The progress of the reaction was monitored by TLC. After completion of the reaction, the reaction mixture was concentrated under reduced pressure and the resultant crude material purified by trituration with diethyl ether (2×20 mL) to provide (S)-(4-(5-chloro-2-ethoxybenzyl)morpholin-2-yl)methanamine hydrochloride (0.1802, 90.07% yield) as a yellow solid.

LCMS [ESI, M+1]: 284.84 (RT: 0.730 min, Purity: 99.73%),

HPLC: RT: 3.927 min, Purity: 100%,

Chiral HPLC: RT: 2.60 min, Purity: 100%,

Instrument Name: Waters SFC Investigator

Chromatographic separation was conducted with Waters SFC Investigator system with 2998 PDA detector. The column used was CHIRALPAK® IG (250×4.6 mm; 5 ␣m) and the compounds were eluted with, Mobile Phase A: Liq·CO2, Mobile Phase B: 0.1% METHANOLIC AMMONIA in METHANOL-ACETONITRILE (50-50) with 55-45 composition method; with Flow rate=3 ml/min; Column oven temperature 40° C.; ABPR 130 bar, analysis time 5 min.

¹H NMR (400 MHz, CD₃OD): δ 7.63 (d, J=2.2 Hz, 1H), 7.49 (dd, J=8.9, 2.0 Hz, 1H), 7.14 (d, J=8.9 Hz, 1H), 4.55-4.35 (m, 2H), 4.21 (dd, J=13.8, 6.8 Hz, 4H), 3.99 (t, J=12.0 Hz, 1H), 3.59 (d, J=12.4 Hz, 1H), 3.50 (d, J=12.7 Hz, 1H), 3.32-3.21 (m, 2H), 3.07 (dt, J=22.9, 12.4 Hz, 2H), 1.50 (t, J=6.9 Hz, 3H).

### Experimental Protocol for Compound 29

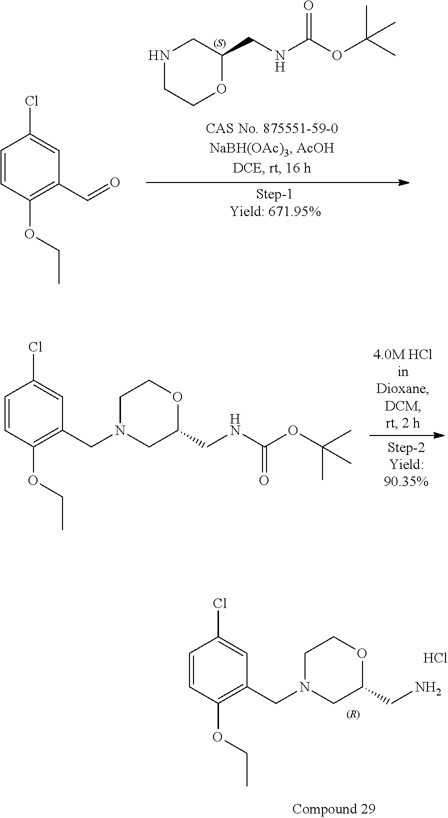

### Step-1: Synthesis of tert-butyl (R)-((4-(5-chloro-2-ethoxybenzyl)morpholin-2-yl)methyl) carbamate

### Procedure

To a stirred solution of 5-chloro-2-ethoxybenzaldehyde (0.2 g, 1.08 mmol) in DCE (4 mL) at room temperature, tert-butyl (S)-(morpholin-2-ylmethyl) carbamate (0.281 g, 1.29 mmol) was added. The reaction mixture was stirred at room temperature for 2 h. Sodium triacetoxyborohydride (0.68 g, 3.2 mmol) was added portion-wise to the reaction mixture at 0° C. The reaction was stirred at room temperature for 16 h. The progress of the reaction was monitored by TLC and LCMS analysis. After completion of the reaction, the reaction mixture was poured into a saturated solution of sodium bicarbonate (20 mL) and extracted with CH₂Cl₂ (3×10 mL). The combined organic fractions were washed with water (20 mL), dried over sodium sulphate and concentrated under reduced pressure. The crude material was purified by flash column chromatography (SiO₂; 17% ethyl acetate in hexane) to provide tert-butyl (R)-((4-(5-chloro-2-ethoxybenzyl) morpholin-2-yl) methyl) carbamate (0.32, 71.95% yield) as a light yellow liquid.

LCMS [ESI, M+1]: 384.96 (RT: 1.400 min, Purity: 100%).

### Step-2: Synthesis of (R)-(4-(5-chloro-2-ethoxybenzyl)morpholin-2-yl)methanamine hydrochloride (Compound 29)

### Procedure

To a stirred solution of tert-butyl (R)-((4-(5-chloro-2-ethoxybenzyl) morpholin-2-yl) methyl) carbamate (0.3 g, 0.7 mmol) in CH₂Cl₂ (3 mL, 10V) at 0° C., 4M HCl in dioxane (1.5 mL, 5V) was added. The reaction mixture was stirred at room temperature for 2 h. After completion of the reaction, the reaction mixture was concentrated under reduced pressure and the resulting crude material purified by trituration with diethyl ether (2×20 mL) to provide (R)-(4-(5-chloro-2-ethoxybenzyl)morpholin-2-yl)methanamine hydrochloride (0.212, 90.35% yield) as an off-white solid.

LCMS [ESI, M+1]: 284.8 (RT: 0.748 min, Purity: 97.65%),

HPLC: RT: 3.927 min, Purity: 98.89%,

Chiral HPLC: RT: 2.60 min, Purity: 100%,

Instrument Name: Waters SFC Investigator

Chromatographic separation was conducted with Waters SFC Investigator system with 2998 PDA detector. The column used was CHIRALPAK® IG (250×4.6 mm; 5 ␣m) and the compounds were eluted with, Mobile Phase A: Liq·CO2, Mobile Phase B: 0.1% METHANOLIC AMMONIA in METHANOL-ACETONITRILE (50-50) with 55-45 composition method; with Flow rate=3 ml/min; Column oven temperature 40° C.; ABPR 130 bar, analysis time 5 min.

¹H NMR (400 MHz, CD₃OD): δ7.63 (d, J=2.5 Hz, 1H), 7.49 (dd, J=8.9, 2.6 Hz, 1H), 7.14 (d, J=8.9 Hz, 1H), 4.49-4.37 (m, 2H), 4.21 (dd, J=13.9, 6.9 Hz, 4H), 3.99 (t, J=11.7 Hz, 1H), 3.54 (dd, J=34.1, 12.6 Hz, 2H), 3.31-3.22 (m, 2H), 3.13-2.98 (m, 2H), 1.50 (t, J=7.0 Hz, 3H).

### Experimental Protocol for Compound 30

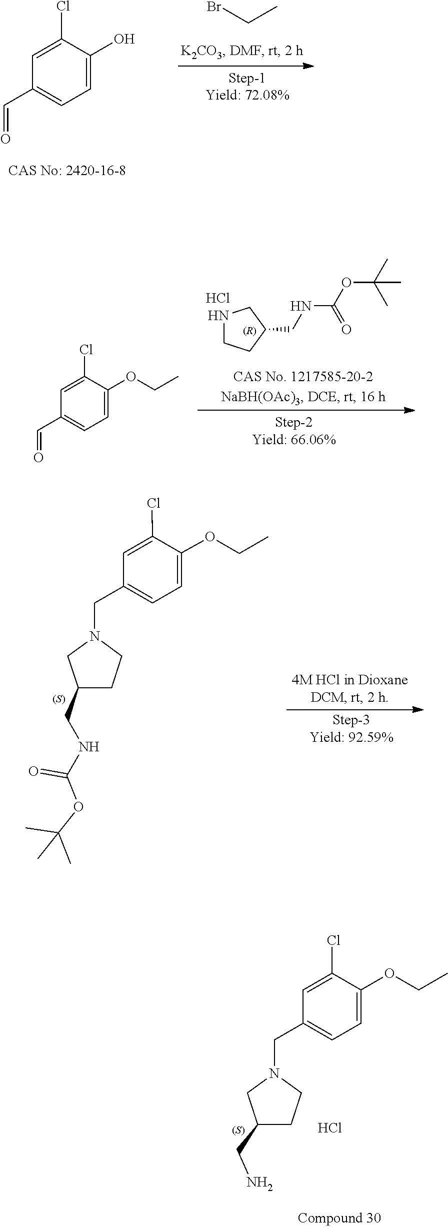

### Step-1: Synthesis of 3-chloro-4-ethoxybenzaldehyde

### Procedure

To a stirred solution of 3-chloro-4-hydroxybenzaldehyde (10.0 g, 63.86 mmol) in DMF (100 mL, 10V) at room temperature, K₂CO₃ (26.4 g, 191.60 mmol) was added. The reaction mixture was stirred at room temperature for 30 min. Then 1-bromoethane (7.7 g, 70.25 mmol) was added to the reaction mixture. The reaction mixture was stirred at room temperature for 1.5 h. The progress of the reaction was monitored by TLC. After completion of the reaction, the reaction mixture was poured into ice cold water (500 mL). The solid precipitate was isolated by filtration, washed with cold water (3×100 mL) and dried under reduced pressure to provide 3-chloro-4-ethoxybenzaldehyde (8.52, 72.08% yield as a white solid.

LCMS [ESI, M+1]: 184.8 (RT: 1.900 min, Purity: 99.24%), ¹H NMR (400 MHz, d₆-DMSO) δ: 9.87 (s, 1H), 7.96 (d, J=2.0 Hz, 1H), 7.89 (dd, J=8.5, 2.0 Hz, 1H), 7.36 (d, J=8.5 Hz, 1H), 4.26 (q, J=7.0 Hz, 2H), 1.40 (t, J=7.0 Hz, 3H).

### Step-2: Synthesis of tert-butyl (S)-((1-(3-chloro-4-ethoxybenzyl)pyrrolidin-3-yl)methyl) carbamate)

### Procedure

To a stirred solution of 3-chloro-4-ethoxybenzaldehyde (2.5 g, 13.54 mmol) in DCE (250 mL, 10V) at room temperature, tert-butyl (R)-(pyrrolidin-3-ylmethyl)carbamate hydrochloride (3.8 g, 16.24 mmol) was added. The reaction mixture was stirred at room temperature for 2 h. Sodium triacetoxyborohydride (8.6 g, 40.62 mmol) was added to the reaction mixture portion-wise at 0° C. The reaction was stirred at room temperature for 16 h. The progress of the reaction was monitored by TLC and LCMS analysis. After completion of the reaction, the reaction mixture was poured into a saturated solution of sodium bicarbonate (200 mL) and extracted with CH₂Cl₂ (3×100 mL). The combined organic fractions were washed with water (300 mL), dried over sodium sulphate and concentrated under reduced pressure. The crude material was purified by column chromatography (SiO₂; 45-50% EtOAc in hexane) to provide tert-butyl (S)-((1-(3-chloro-4-ethoxybenzyl)pyrrolidin-3-yl)methyl)carbamate (3.3 g, 66.06% yield) as a yellow sticky solid.

LCMS [ESI, M+1]: 369.0 (RT: 1.403 min, Purity: 91.41%),

Chiral HPLC: RT: 7.40 min, Purity: 100.00% ¹H NMR (400 MHz, d₆-DMSO) δ: 7.32 (t, J=4.8 Hz, 1H), 7.21 (dd, J=8.4, 2.0 Hz, 1H), 7.08 (d, J=8.4 Hz, 1H), 6.88 (t, J=5.5 Hz, 1H), 4.09 (q, J=13.5 Hz, 2H), 3.46 (d, J=7.9 Hz, 2H), 2.97-2.82 (m, 2H), 2.50-2.35 (m, 3H), 2.29-2.12 (m, 2H), 1.90-1.76 (m, 2H), 1.44-1.35 (m, 12H).

### Step-3: Synthesis of (S)-(1-(3-chloro-4-ethoxybenzyl)pyrrolidin-3-yl)methanamine hydrochloride (Compound 30)

### Procedure

To a stirred solution of tert-butyl (S)-((1-(3-chloro-4-ethoxybenzyl) pyrrolidin-3-yl)methyl)carbamate (3.3 g, 8.96 mmol) in CH₂Cl₂ (33 mL, 10V) at room temperature, 4M HCl in dioxane (16.5 mL, 5V) was added. The reaction mixture was stirred at room temperature for 2 h. The progress of the reaction was monitored by TLC and LCMS analysis. After completion of the reaction, the reaction mixture was concentrated under reduced pressure and the crude material purified by trituration with CH₂Cl₂ (3×50 mL) to provide (S)-(1-(3-chloro-4-ethoxybenzyl)pyrrolidin-3-yl)methanamine hydrochloride (2.52, 92.59%) as a white solid.

LCMS [ESI, M+1]: 268.9 (RT: 0.802 min, Purity: 99.78%),

HPLC: RT: 4.09 min, Purity: 98.14%

Chiral HPLC: RT: 10.195 min, Purity: 100.00%

Instrument Name: Shimadzu LC-20 AD

Chromatographic separation was conducted with Shimadzu LC-20 AD system with DAD detector. The column used was CHIRALPAK® IG (250×4.6 mm; 5 ␣m) and the compounds were eluted with, Mobile Phase A: 0.1% Methanolic ammonia in n-Heptane, Mobile Phase B: 2-Propanol with an isocratic method (40:60), with Flow rate=1 ml/min; analysis time 30 min.

¹H NMR (400 MHz, CD₃OD) δ: 7.65 (s, 1H), 7.50 (d, J=6.9 Hz, 1H), 7.17 (d, J=8.5 Hz, 1H), 4.41 (d, J=24.1 Hz, 2H), 4.18 (q, J=12.5 Hz, 2H), 3.74-3.46 (m, 3H), 3.23-3.01 (m, 3H), 2.82 (m, 1H), 2.38 (m, 1H), 1.94 (m, 1H), 1.56 (t, J=11.9 Hz, 3H).

### Experimental Protocol for Compound 31

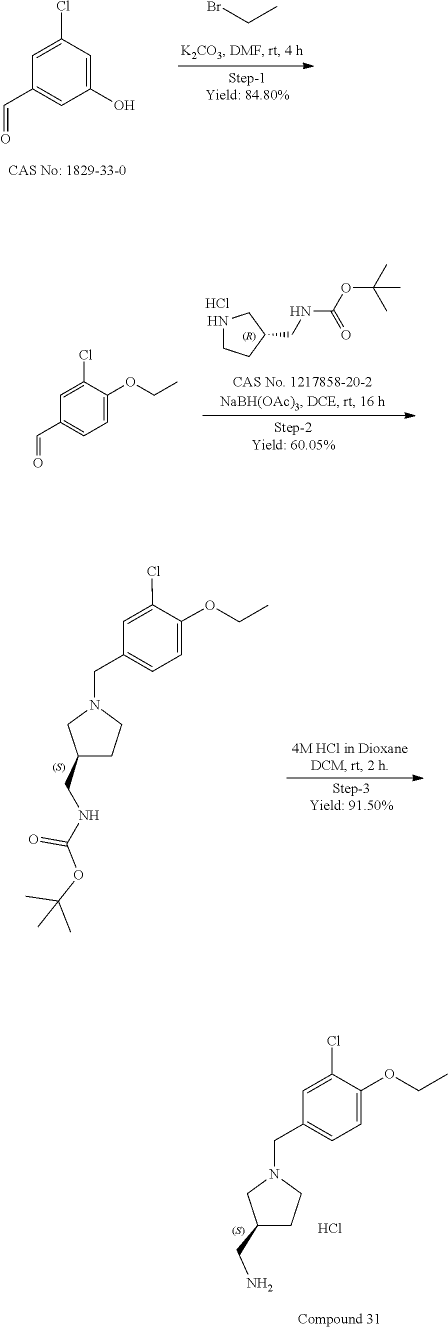

### Step-1: Synthesis of 3-chloro-5-ethoxybenzaldehyde

### Procedure

To a stirred solution of 3-chloro-5-hydroxybenzaldehyde (0.5 g, 3.18 mmol) in DMF (5 mL, 10V) at room temperature, K₂CO₃ (1.3 g, 9.55 mmol) was added. The reaction was stirred for 30 min, then 1-bromoethane (0.353 g, 3.50 mmol) was added to the reaction mixture. The reaction mixture was stirred at room temperature for 4 h. The progress of the reaction was monitored by TLC. After completion of the reaction, the reaction mixture was poured into ice cold water (80 mL). The solid precipitate was isolated by filtration, washed with cold water (2×20 mL), and dried under reduced pressure to provide 3-chloro-5-ethoxybenzaldehyde (0.5 g, 84.80% yield) as a white solid.

¹H NMR (400 MHz, d₆-DMSO) δ: 9.94 (s, 1H), 7.49 (d, J=14.8 Hz, 1H), 7.38 (d, J=9.0 Hz, 1H), 7.34 (dd, J=16.2, 2.0 Hz, 1H), 4.13 (q, J=7.0 Hz, 2H), 1.34 (t, J=7.0 Hz, 3H).

### Step-2: Synthesis of tert-butyl (S)-((1-(3-chloro-5-ethoxybenzyl)pyrrolidin-3-yl)methyl) carbamate

### Procedure

To a stirred solution of 3-chloro-5-ethoxybenzaldehyde (0.5 g, 2.70 mmol) in DCE (10 mL, 20V) at room temperature, tert-butyl (R)-(pyrrolidin-3-ylmethyl)carbamate hydrochloride (0.769 g, 3.24 mmol) was added. The reaction mixture was stirred at room temperature for 2 h. Sodium triacetoxyborohydride (1.7 g, 8.12 mmol) was added to the reaction mixture portion-wise at 0° C. The reaction mixture was stirred at room temperature for 16 h. The progress of the reaction was monitored by TLC and LCMS analysis. After completion of the reaction, the reaction mixture was poured into a saturated solution of sodium bicarbonate (70 mL) and extracted with CH₂Cl₂ (3×40 mL). The combined organic fractions were washed with water (50 mL), dried over sodium sulphate and concentrated under reduced pressure. The crude product material was purified by column chromatography (SiO₂; 4-5% MeOH in CH₂Cl₂) to provide tert-butyl (S)-((1-(3-chloro-5-ethoxybenzyl)pyrrolidin-3-yl)methyl)carbamate (0.6 g, 60.05% yield) as a yellow sticky solid.

LCMS [ESI, M+1]: 369.0 (RT: 1.532 min, Purity: 100%),

Chiral HPLC: RT: 2.83 min, Purity: 100.00%

¹H NMR (400 MHz, d₆-DMSO) δ: 6.86 (t, J=4.8 Hz, 3H), 4.03 (q, J=12.5 Hz, 2H), 3.46 (d, J=7.9 Hz, 2H), 2.97-2.82 (m, 2H), 2.50-2.35 (m, 4H), 2.29-2.12 (m, 2H), 1.83-1.79 (m, 2H), 1.35 (s, 9H), 1.32 (t, J=7.0 Hz, 3H).

### Step-3: Synthesis of (S)-(1-(3-chloro-5-ethoxybenzyl)pyrrolidin-3-yl)methanamine hydro chloride (Compound 31)

### Procedure

To a stirred solution of tert-butyl (S)-((1-(3-chloro-5-ethoxybenzyl)pyrrolidin-3-yl)methyl) carbamate (0.6 g, 1.63 mmol) in CH₂Cl₂ (6 mL, 10V) at room temperature, 4M HCl in dioxane (3 mL, 5V) was added. The reaction mixture was stirred at room temperature for 2 h. The progress of the reaction was monitored by TLC and LCMS analysis. After completion of the reaction, the reaction mixture was concentrated under reduced pressure and the crude material was purified by trituration with CH₂Cl₂ (2×30 mL) to provide (S)-(1-(3-chloro-5-ethoxybenzyl)pyrrolidin-3-yl)methanamine hydrochloride (0.42, 91.50%) as a white solid.

LCMS [ESI, M+1]: 269.0 (RT: 0.800 min, Purity: 99.70%),

HPLC: RT: 4.01 min, Purity: 99.81%

Chiral HPLC: RT: 2.88 min, Purity: 100.00%

Instrument Name: Waters SFC Investigator

Chromatographic separation was conducted with Waters SFC Investigator system with 2998 PDA detector. The column used was CHIRALPAK® IG (250×4.6 mm; 5 ␣m) and the compounds were eluted with, Mobile Phase A: Liq·CO2, Mobile Phase B: 0.1% METHANOLIC AMMONIA in METHANOL-ACETONITRILE (50-50) with 60-40 composition method; with Flow rate=3 ml/min; Column oven temperature 40° C.; ABPR 130 bar, analysis time 6 min.

¹H NMR (400 MHz, CD₃OD) δ: 7.16 (dd, J=34.6, 17.7 Hz, 3H), 4.41 (m, 2H), 4.12 (q, J=7.0 Hz, 2H), 3.80-3.56 (m, 2H), 3.44 (m, 2H), 3.17 (t, J=9.9 Hz, 2H), 3.07 (dd, J=20.8, 9.2 Hz, 1H), 3.02-2.66 (m, 1H), 2.56-2.24 (m, 1H), 1.42 (t, J=7.0 Hz, 3H).

### Experimental Protocol for Compound 32

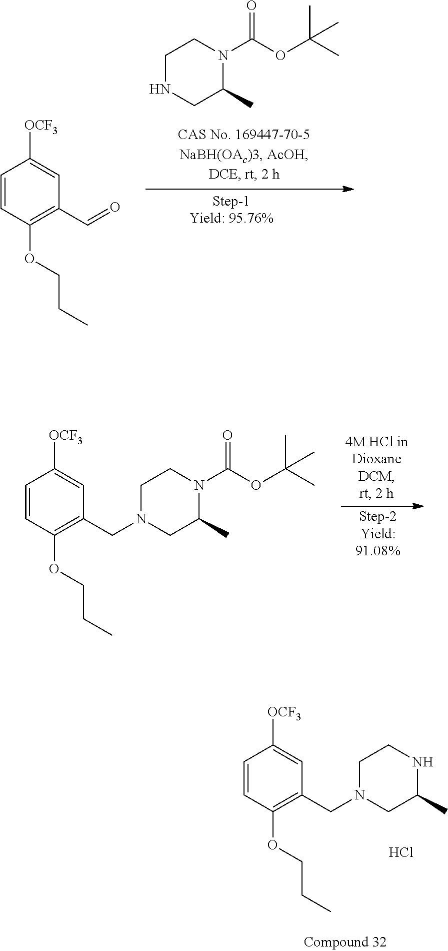 [SMILES: C[C@H]1CNCCN1C(=O)OC(C)(C)C ; CCCOc1ccc(OC(F)(F)F)cc1C=O ; CCCOc1ccc(OC(F)(F)F)cc1CN1CCN(C(=O)OC(C)(C)C)[C@@H](C)C1 ; CCCOc1ccc(OC(F)(F)F)cc1CN1CCN[C@@H](C)C1 ; Cl]

### Step-1: Synthesis of tert-butyl (S)-2-methyl-4-(2-propoxy-5-(trifluoromethoxy) benzyl) piperazine-1-carboxylate

### Procedure

To a stirred solution of 2-propoxy-5-(trifluoromethoxy) benzaldehyde (0.3 g, 1.20 mmol) in DCE (5 mL) at room temperature, tert-butyl tert-butyl (S)-2-methylpiperazine-1-carboxylate (0.3 g, 1.45 mmol) was added. The reaction mixture was stirred at room temperature for 1 h. Then sodium triacetoxyborohydride (0.732 g, 3.624 mmol) was added portion-wise into reaction mixture at 0° C. The reaction was stirred at room temperature for 1 h. The progress of the reaction was monitored by TLC and LCMS analysis. After completion of the reaction, the reaction mixture was poured into a saturated sodium bicarbonate solution (30 mL) and extracted with CH₂Cl₂ (3×30 mL). The combined organic fractions were washed with cold water (2×10 mL), dried over sodium sulphate and concentrated under reduced pressure. The crude material was purified by column chromatography (SiO₂; 15% ethyl acetate in hexane) to provide tert-butyl (S)-2-methyl-4-(2-propoxy-5-(trifluoromethoxy) benzyl) piperazine-1-carboxylate (0.52, 95.76% yield) as a colourless sticky solid LCMS [ESI, M+1]: 433.12 (RT: 1.830 min, Purity: 93.73%).

### Step-2: Synthesis of (S)-3-methyl-1-(2-propoxy-5-(trifluoromethoxy)benzyl) piperazine hydrochloride (Compound 32)

### Procedure

To a stirred solution of tert-butyl (S)-2-methyl-4-(2-propoxy-5-(trifluoromethoxy) benzyl) piperazine-1-carboxylate (0.5 g, 0.11 mmol) in CH₂Cl₂ (5 mL) at 0° C., 4M HCl in dioxane (2.5 mL, 5V) was added. The reaction mixture was stirred at room temperature for 2 h. The progress of the reaction was monitored by TLC and LCMS analysis. After completion of the reaction, the reaction mixture was concentrated under reduced pressure and the crude material purified by trituration with diethyl ether (2×25 mL) to provide (S)-3-methyl-1-(2-propoxy-5-(trifluoromethoxy)benzyl) piperazine hydrochloride (0.352, 91.08% yield as an off-white solid.

LCMS [ESI, M+1]: 332.90 (RT: 1.309 min, Purity: 95.88%),

HPLC: RT: 4.793 min, Purity: 99.70%,

Chiral HPLC: RT: 2.06 min, Purity: 94.74%

Instrument Name: Waters SFC Investigator

Chromatographic separation was conducted with Waters SFC Investigator system with 2998 PDA detector. The column used was CHIRALPAK® IG (250×4.6 mm; 5 ␣m) and the compounds were eluted with, Mobile Phase A: Liq·CO2, Mobile Phase B: 0.1% METHANOLIC AMMONIA in METHANOL-ACETONITRILE (50-50) with 70-30 composition method; with Flow rate=3 ml/min; Column oven temperature 40° C.; ABPR 130 bar, analysis time 5 min.

¹H NMR (400 MHz, CD₃OD): δ 7.58 (d, J=2.8 Hz, 1H), 7.45 (dd, J=2 Hz, J=2.4 Hz, 1H), 7.24 (d, J=9.2 Hz, 1H), 4.50 (s, 2H), 4.13 (t, J=6.8 Hz, 2H), 3.85-3.78 (m, 1H), 3.77-3.72 (m, 3H), 3.61-3.55 (m, 2H), 3.48-3.44 (m, 1H), 1.98-1.89 (m, 2H), 1.44 (d, J=6.8 Hz, 3H), 1.11 (t, J=7.2 Hz, 3H).

### Experimental Protocol for Compound 33

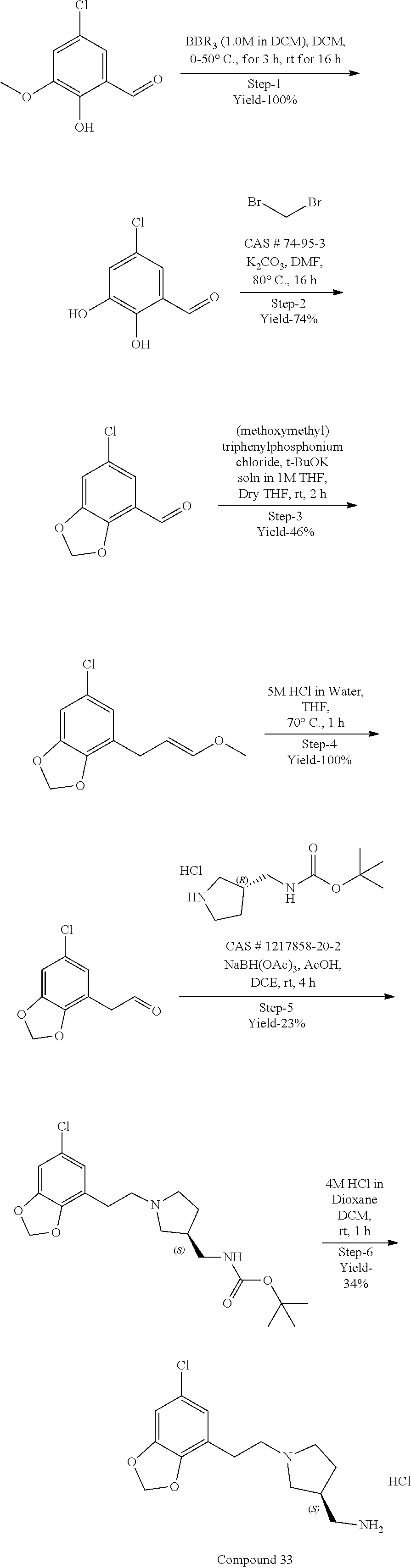

### Step-1: Synthesis of 5-chloro-2,3-dihydroxybenzaldehyde

### Procedure

To a stirred solution of 5-chloro-2-hydroxy-3-methoxybenzaldehyde, (1.0 g, 5.35 mmol) in CH₂Cl₂ (15 mL) at 0° C., BBr₃ (LOM in CH₂Cl₂) (16.0 mL, 3.0 eq) was added. The reaction mixture was stirred at 50° C. for 3 h and then at room temperature for 16 h. The progress of the reaction was monitored by TLC. After completion of the reaction, the reaction mixture was concentrated under reduced pressure. The crude material was quenched with ice-cold water (60 mL) and extracted with ethyl acetate (3×30 mL). The combined organic fractions were dried over sodium sulphate and concentrated under reduced pressure. The crude material was triturated with n-hexane (2×20 mL) to provide 5-chloro-2,3-dihydroxybenzaldehyde (1.0 g, 100% yield) as a green solid which was used directly in the next step.

LCMS [ESI, M+1]: 170.93 (RT: 1.371 min, Purity: 94.61%).

### Step-2: Synthesis of 6-chlorobenzo[d][1,3]dioxole-4-carbaldehyde

### Procedure

To a stirred solution of 5-chloro-2,3-dihydroxybenzaldehyde (1.0 g, 5.79 mmol) in anhydrous DMF (10 mL, 10V) at 0° C., K₂CO₃ (2.4 g, 17.3 mmol), then dibromomethane (3.0 g, 17.3 mmol) were added. The reaction mixture was stirred at 80° C. for 16 h. The progress of the reaction was monitored by TLC analysis. After completion of the reaction, the reaction mixture was poured into ice cold water (100 mL). The solid that formed was isolated by filtration and dried under reduced pressure to provide 6-chlorobenzo[d][1,3]dioxole-4-carbaldehyde (0.8 g, 74.0% yield) as a brown solid which was used directly in the next step.

¹H NMR (400 MHz, d₆-DMSO): δ 9.98 (s, 1H), 7.38-7.28 (dd, 2H), 6.28 (s, 2H).

### Step-3: Synthesis of (E)-6-chloro-4-(3-methoxyallyl) benzo[d][1,3] dioxole

### Procedure

To a stirred solution of (methoxymethyl)triphenyl phosphonium chloride, (2.32 g, 6.70 mmol) in anhydrous THF (20 mL, 40V) at 0° C., t-BuOK solution in 1M THF (12.5 mL) was added. The reaction mixture was stirred at room temperature for 1 h. Then 6-chlorobenzo[d][1,3]dioxole-4-carbaldehyde (0.5 g, 2.70 mmol) dissolved in THF (1 mL) was added drop wise into reaction mixture at 0° C. The reaction was stirred at room temperature for 1 h. The progress of the reaction was monitored by TLC. After completion of the reaction, the reaction mixture was concentrated under reduced pressure and the crude material purified by normal phase column chromatography (Aluminum oxide neutral; 0-2% ethyl acetate in hexane) to provide (E)-6-chloro-4-(3-methoxyallyl) benzo[d][1,3]dioxole (0.28 g, 46.00% yield) as a light yellow liquid which was used directly in the next step without analysis.

### Step-4: Synthesis of 2-(6-chlorobenzo[d][1,3]dioxol-4-Yl)acetaldehyde

### Procedure

To a stirred solution (E)-6-chloro-4-(3-methoxyallyl) benzo[d][1,3]dioxole (0.27 g, 1.19 mmol) in THF (2.7 mL, 10V) at room temperature, 5M HCl aqueous solution (2.3 mL, 5V) was added. The reaction mixture was heated at 70° C. for 1 h. The progress of the reaction was monitored by TLC analysis. After completion of the reaction; the reaction mixture was poured into a saturated solution of sodium bicarbonate (50 mL) and extracted with ethyl acetate (3×30 mL). The combined organic fractions were washed with water (2×10 mL), dried over sodium sulphate and concentrated under reduced pressure to provide 2-(6-chlorobenzo[d][1,3]dioxol-4-yl)acetaldehyde (0.25 g, 100% yield) as an off-white sticky solid which was confirmed by TLC analysis using 2,4-DNP Stain and used directly in the next step.

### Step-5: Synthesis of tert-butyl (S)-((1-(2-(6-chlorobenzo[d][1,3] dioxol-4-yl)ethyl)pyrrolidin-3-yl)methyl) carbamate

### Procedure

To a stirred solution of 2-(6-chlorobenzo[d][1,3]dioxol-4-yl)acetaldehyde (0.23 g, 1.15 mmol) in DCE (5 mL, 20V) at room temperature, tert-butyl (R)-(pyrrolidin-3-ylmethyl) carbamate hydrochloride (0.327 g, 1.38 mmol) and Acetic acid (0.003 mL, 0.05V) were added. The reaction mixture was stirred at room temperature for 2 h. Sodium triacetoxyborohydride (0.738 g, 3.48 mmol) was added portion-wise to the reaction mixture at 0° C. After addition, the reaction was stirred at room temperature for 3 h. The progress of the reaction was monitored by TLC and LCMS analysis. After completion of the reaction, the reaction mixture was poured into a saturated solution of sodium bicarbonate (30 mL) and extracted with CH₂Cl₂ (3×30 mL). The combined organic fractions were washed with water (2×15 mL), dried over sodium sulphate and concentrated under reduced pressure. The crude material was purified by column chromatography (neutral alumina; 35% ethyl acetate in hexane) to provide tert-butyl (S)-((1-(2-(6-chlorobenzo[d][1,3]dioxol-4-yl)ethyl)pyrrolidin-3-yl)methyl)carbamate (0.1 g, 23% yield) as a light yellow liquid.

LCMS [ESI, M+1]: 382.96 (RT: 1.441 min, Purity: 63.71%).

### Step-6: Synthesis of (S)-(1-(2-(6-chlorobenzo[d][1,3]dioxol-4-yl)ethyl)pyrrolidin-3-yl)methanamine hydrochloride (Compound 33)

### Procedure

To a stirred solution of tert-butyl (S)-((1-(2-(6-chlorobenzo[d][1,3]dioxol-4-yl)ethyl)pyrrolidin-3-yl)methyl)carbamate (0.1 g, 0.26 mmol) in CH₂Cl₂ (1.0 mL, 10V) at 0° C., 4M HCl in dioxane (0.8 mL, 5.0V) was added. The reaction mixture was stirred at room temperature for 1 h. After completion of the reaction, the reaction mixture was concentrated under reduced pressure and the crude material purified by preparative HPLC (0.05% HCl in water/CH₃CN) to provide (S)-(1-(2-(6-chlorobenzo[d][1,3]dioxol-4-yl)ethyl) pyrrolidin-3-yl)methanamine hydrochloride (0.025 g, 34% yield) as a white sticky solid.

LCMS [ESI, M+1]: 282.79 (RT: 0.841 min, Purity: 99.53%).

HPLC: RT: 4.007 min, Purity: 99.30%,

Chiral HPLC: RT: 2.49 min, Purity: 100%,

Instrument Name: Waters SFC Investigator

Chromatographic separation was conducted with Waters SFC Investigator system with 2998 PDA detector. The column used was CHIRALPAK® IG (250×4.6 mm; 5 ␣m) and the compounds were eluted with, Mobile Phase A: Liq·CO2, Mobile Phase B: 0.1% METHANOLIC AMMONIA in METHANOL-ACETONITRILE (50-50) with 70-30 composition method; with Flow rate=3 ml/min; Column oven temperature 40° C.; ABPR 130 bar, analysis time 5 min.

¹H NMR (400 MHz, CD₃OD): δ 6.85 (dd, J=11.6 Hz, 2H), 6.07 (s, 2H), 3.91-3.66 (m, 2H), 3.71-3.59 (m, 1H), 3.63-3.50 (m, 3H), 3.33 (m, 4H), 2.99-2.77 (m, 1H), 2.47-2.32 (m, 1H), 2.03-1.87 (m, 2H).

### Experimental Protocols for Compound 34 and Compound 35

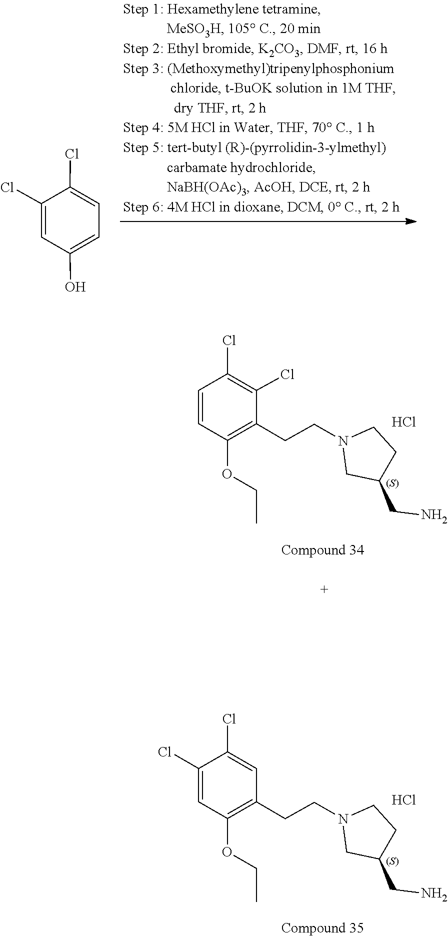

### Step-1: Synthesis of 4,5-dichloro-2-hydroxybenzaldehyde

### Procedure

To a stirred solution of 3,4-dichlorophenol (5.0 g, 30.67 mmol) in Methyl sulphonic acid (30 mL) at room temperature, hexamethylene tetramine (4.68 g, 33.43 mmol) was added.

The reaction mixture was heated at 105° C. for 20 min. The progress of the reaction was monitored by TLC and LCMS analysis. After completion of the reaction, the reaction mixture was poured into cold water (80 mL). The solid precipitate was isolated by filtration and dried under reduced pressure to provide 4,5-dichloro-2-hydroxy benzaldehyde (3.52, 59.73% yield) as an off-white solid.

¹H NMR (400 MHz, d₆-DMSO): δ 10.33 (s, 1H), 7.77 (dd, J=8.9, 2.8 Hz, 1H), 7.24 (d, J=2.7 Hz, 1H), 7.04 (d, J=9.0 Hz, 1H). Based on observations at the end of Step 5 (see below), it can be inferred that this material was contaminated with the corresponding 2,3-dichloro isomer.

### Step-2: Synthesis of 4,5-dichloro-2-ethoxybenzaldehyde

### Procedure

To a stirred solution of 4,5-dichloro-2-hydroxybenzaldehyde (3.5 g, 18.32 mmol)* in DMF (35 mL) at room temperature, Potassium carbonate (7.6 g, 54.97 mmol) was added. The reaction mixture was stirred at room temperature for 1 h. Ethyl bromide (2.4 g, 21.98 mmol) was added to the reaction mixture. The reaction mixture was stirred at room temperature for 16 h. The progress of the reaction was monitored by TLC and LCMS analysis. After completion of the reaction, the reaction mixture was poured into ice cold water (80 mL). A solid precipitate was obtained, which was isolated by filtration and dried under reduced pressure. The crude material was purified by column chromatography (SiO₂; 10% ethyl acetate in hexane) to provide 4,5-dichloro-2-ethoxybenzaldehyde (1.0 g, 24.91% yield) as a white solid.* * Based on observations at the end of Step 5 (see below), it can be inferred that this material was contaminated with the corresponding 2,3-dichloro isomer. ¹H NMR (400 MHz, d₆-DMSO): δ 10.26 (s, 1H), 7.79 (dd, J=8.9, 2.8 Hz, 1H), 7.59 (d, J=2.7 Hz, 1H), 4.25 (t, J=7.0 Hz, 2H), 1.39 (t, J=7.0 Hz, 3H).

### Step-3: Synthesis of (E)-1,2-dichloro-4-ethoxy-5-(3-methoxyallyl)benzene

### Procedure

To a stirred solution of (Methoxymethyl)triphenyl phosphonium chloride (1.95 g, 5.70 mmol) in anhydrous THF (10 mL) at room temperature, t-BuOK in 1M THF (11.4 mL, 11.41 mmol) was added. The reaction mixture was stirred at room temperature for 1 h. 4,5-dichloro-2-ethoxybenzaldehyde (0.5 g, 2.28 mmol)* (dissolved in anhydrous THF) was added drop-wise to the reaction mixture at room temperature. The reaction was stirred at room temperature for 1 h. The progress of the reaction was monitored by TLC. After completion of the reaction, the reaction mixture was concentrated under reduced pressure and the crude material purified by column chromatography (alumna oxide neutral; 0-2% ethyl acetate in hexane) to provide (E)-1,2-dichloro-4-ethoxy-5-(3-methoxyallyl)benzene (0.482, 80.53% yield)** as a light yellow liquid. * Based on observations at the end of Step 5 (see below), it can be inferred that this material was contaminated with the corresponding 2,3-dichloro isomer.** Based on observations at the end of Step 5 (see below), it can be inferred that this material was contaminated with the corresponding 4,5-dichloro isomer.

### Step-4: Synthesis of 2-(4,5-dichloro-2-ethoxyphenyl)acetaldehyde

### Procedure

To a stirred solution of (E)-1,2-dichloro-4-ethoxy-5-(3-methoxyallyl)benzene (0.48 g, 1.93 mmol)* in THF (4.8 mL) at room temperature, 5M HCl in water (2.4 mL, 5V) was added. The reaction mixture was stirred at 70° C. for 1 h. The progress of the reaction was monitored by TLC. After completion of the reaction, the reaction mixture was poured into a saturated solution of sodium bicarbonate (60 mL) and extracted with ethyl acetate (3×20 mL). The combined organic fractions were dried over sodium sulphate and concentrated under reduced pressure to provide 2-(4,5-dichloro-2-ethoxyphenyl)acetaldehyde (0.42, 93.36% yield)** as a light yellow liquid.

¹H NMR (400 MHz, d₆-DMSO): δ 9.63 (t, J=1.5 Hz, 1H), 7.34-7.28 (m, 1H), 7.08-6.99 (m, 1H), 3.91 (q, J=6.6 Hz, 2H), 3.73-3.64 (m, 2H), 0.95 (t, J=9.2 Hz, 3H). *Based on observations at the end of Step 5 (see below), it can be inferred that this material was contaminated with the corresponding 4,5-dichloro isomer.** Based on observations at the end of Step 5 (see below), it can be inferred that this material was contaminated with the corresponding 2,3-dichloro isomer.

### Step-5: Synthesis of tert-butyl (S)-((1-(4,5-dichloro-2-ethoxyphenethyl)pyrrolidin-3-yl)methyl) carbamate

### Procedure

To a stirred solution of 2-(4,5-dichloro-2-ethoxyphenyl)acetaldehyde (0.4 g, 1.78 mmol)* in DCE (8 mL) at room temperature, tert-butyl (R)-(pyrrolidin-3-ylmethyl)carbamate hydrochloride (0.507 g, 2.14 mmol) was added. The reaction mixture was stirred at room temperature for 1 h. Sodium triacetoxyborohydride (1.3 g, 5.35 mmol) was added portion-wise to the reaction mixture at 0° C. The reaction was stirred at room temperature for 1 h. The progress of the reaction was monitored by TLC and LCMS analysis. After completion of the reaction, the reaction mixture was poured into a saturated solution of sodium bicarbonate (40 mL) and extracted with CH₂Cl₂ (3×20 mL). The combined organic fractions were washed with water (30 mL), dried over sodium sulphate and concentrated under reduced pressure. The crude material was purified by column chromatography (Aluminum oxide neutral; 25-30% ethyl acetate in hexane) to provide tert-butyl (S)-((1-(4,5-dichloro-2-ethoxyphenethyl)pyrrolidin-3-yl)methyl)carbamate (0.42, 55.58% yield as a light yellow liquid. LCMS analysis indicated that this material was contaminated with another compound of the same molecular weight; this material was used in the next step. * Based on observations at the end of Step 5, it can be inferred that this material was contaminated with the corresponding 2,3-dichloro isomer.

LCMS [ESI, M+1]: Peak-1: 417.4 (RT: 7.88 min, Purity: 37.13%),
Peak-2: 417.4 (RT: 8.03 min, Purity: 62.87%).

### Step-6: Synthesis of (S)-(1-(2,3-dichloro-6-ethoxyphenethyl)pyrrolidin-3-yl)methanamine hydrochloride (Compound 34) and (S)-(1-(4,5-dichloro-2-ethoxyphenethyl)pyrrolidin-3-yl)methanamine hydrochloride (Compound 35)

### Procedure

To a stirred solution of tert-butyl (R)-((1-(4,5-dichloro-2-ethoxyphenethyl)pyrrolidin-3-yl)methyl)carbamate (0.4 g, 1.06 mmol) (contaminated with the corresponding 2,3-dichloro isomer) in CH₂Cl₂ (4.0 mL) at 0° C., 4M HCl in dioxane (2.5 mL, 5V) was added. The reaction mixture was stirred at 0° C. for 2 h. The progress of the reaction was monitored by TLC and LCMS analysis. After completion of the reaction, the reaction mixture was concentrated under reduced pressure and subjected to preparative HPLC purification (0.05% HCl in water/Acetonitrile). Two compounds were isolated: (S)-(1-(2,3-dichloro-6-ethoxyphenethyl)pyrrolidin-3-yl)methanamine hydrochloride (Compound 34), 0.17 g 55.95% yield as a yellow sticky solid

LCMS [ESI, M+1]: 316.8 (RT: 1.058 min, Purity: 99.39%),

HPLC: RT: 4.547 min, Purity: 98.95%,

Chiral HPLC: RT: 2.35 min, Purity: 100%,

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% METHANOLIC AMMONIA in METHANOL with 70-30 composition method; with Flow rate=3 ml/min; Column oven temperature 40° C.; ABPR 130 bar, analysis time 6 min.

¹H NMR (400 MHz, CD₃OD): δ 7.42 (s, 1H), 7.17 (s, 1H), 4.12 (q, J=7.0 Hz, 2H), 3.60 (d, J=52.0 Hz, 2H), 3.49-3.39 (m, 2H), 3.35-3.32 (m, 2H), 3.17-3.08 (m, 2H), 3.08-3.01 (m, 2H), 2.81 (m, 1H), 2.40 (m, 1H), 1.92 (m, 1H), 1.47 (t, J=7.0 Hz, 3H).

(S)-(1-(4,5-dichloro-2-ethoxyphenethyl)pyrrolidin-3-yl)methanamine hydrochloride (Compound 35), 0.12, 73.68% yield as a yellow sticky solid

LCMS [ESI, M+1]: 316.8 (RT: 1.034 min, Purity: 99.74%),

HPLC: RT: 4.380 min, Purity: 99.28%,

Chiral HPLC: RT: 2.38 min, Purity: 100%,

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% METHANOLIC AMMONIA in METHANOL with 70-30 composition method; with Flow rate=3 ml/min; Column oven temperature 40° C.; ABPR 130 bar, analysis time 6 min.

¹H NMR (400 MHz, CD₃OD): δ 7.45 (d, J=9.0 Hz, 1H), 7.00 (d, J=9.0 Hz, 1H), 4.15 (q, J=7.0 Hz, 2H), 4.01-3.49 (m, 3H), 3.33-3.26 (m, 3H), 3.22-3.06 (m, 4H), 2.83 (m, 1H), 2.41 (m, 1H), 1.94 (m, 1H), 1.47 (t, J=7.0 Hz, 3H).

### Experimental Protocol for Compound 36

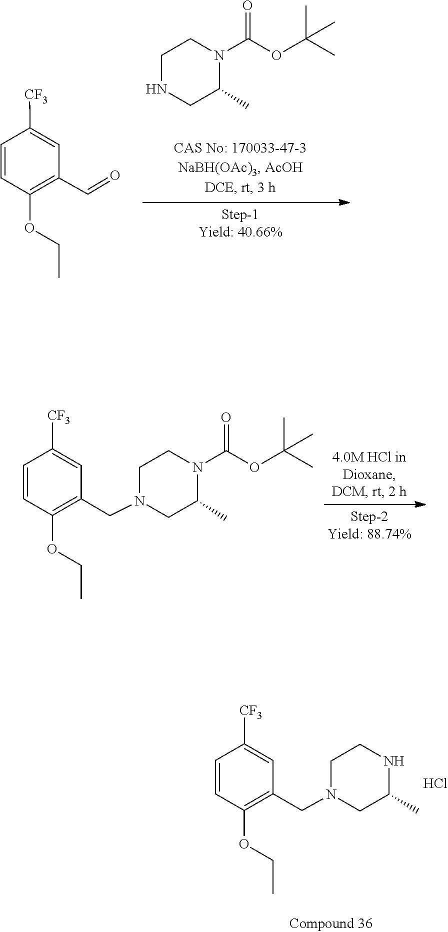 [SMILES: CCOc1ccc(C(F)(F)F)cc1C=O ; C[C@@H]1CNCCN1C(=O)OC(C)(C)C ; CCOc1ccc(C(F)(F)F)cc1CN1CCN(C(=O)OC(C)(C)C)[C@H](C)C1 ; CCOc1ccc(C(F)(F)F)cc1CN1CCN[C@H](C)C1 ; Cl]

### Step-1: Synthesis of tert-butyl (R)-4-(2-ethoxy-5-(trifluoromethyl)benzyl)-2-methylpiperazine-1-carboxylate

### Procedure

To a stirred solution of 2-ethoxy-5-(trifluoromethyl)benzaldehyde (0.2 g, 0.917 mmol) in DCE (2.0 mL) at room temperature, tert-butyl (R)-2-methylpiperazine-1-carboxylate (0.22 g, 1.10 mmol) and acetic acid (0.01 mL, 0.05V) were added. The reaction mixture was stirred at room temperature for 1 h. Then sodium triacetoxyborohydride (0.58 g, 2.751 mmol) was added portion-wise to the reaction mixture at 0° C. The reaction was stirred at room temperature for 2 h. The progress of the reaction was monitored by TLC and LCMS analysis. After completion of the reaction, the reaction mixture was poured into a saturated solution of sodium bicarbonate (30 mL) and extracted with CH₂Cl₂ (3×20 mL). The combined organic fractions were washed with water (30 mL), dried over sodium sulphate and concentrated under reduced pressure. The crude material was purified by flash column chromatography (neutral silica; 20% ethyl acetate in hexane) to provide tert-butyl (R)-4-(2-ethoxy-5-(trifluoromethyl)benzyl)-2-methylpiperazine-1-carboxylate (0.15 g, 40.66% yield) as a white sticky liquid.

LCMS [ESI, M+2]: 403.2 (RT: 1.554 min, Purity: 96.76%).

### Step-2: Synthesis of (R)-1-(2-ethoxy-5-(trifluoromethyl)benzyl)-3-methylpiperazine hydrochloride (Compound 36)

### Procedure

To a stirred solution of tert-butyl (R)-4-(2-ethoxy-5-(trifluoromethyl)benzyl)-2-methylpiperazine-1-carboxylate (0.15 g, 0.373 mmol) in CH₂Cl₂ (1.5 mL, 10V) at 0° C., 4M HCl in dioxane (0.7 mL, 5V) was added. The reaction mixture was stirred at room temperature for 2 h. The progress of the reaction was monitored by TLC. After completion of reaction, the reaction mixture was concentrated under reduced pressure and the crude material triturated with diethyl ether (2×20 mL) to provide (R)-1-(2-ethoxy-5-(trifluoromethyl)benzyl)-3-methylpiperazine hydrochloride (0.1 g, 88.74% yield) as a white solid.

LCMS [ESI, M+1]: 303.0 (RT: 1.084 min, Purity: 95.53%),

HPLC: RT: 4.447 min, Purity: 95.42%,

Chiral HPLC: RT: 1.79 min, Purity: 96.36%,

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% METHANOLIC AMMONIA in METHANOL with 80-20 composition method; with Flow rate=3 ml/min; Column oven temperature 40° C.; ABPR 130 bar, analysis time 5 min.

¹H NMR (400 MHz, CD₃OD) δ 7.94 (t, J=9.9 Hz, 1H), 7.82 (dd, J=8.8, 1.8 Hz, 1H), 7.32 (d, J=8.8 Hz, 1H), 4.55 (s, 2H), 4.31 (q, J=7.0 Hz, 2H), 3.87 (m, 1H), 3.81-3.68 (m, 3H), 3.56 (dt, J=23.1, 12.3 Hz, 2H), 3.47-3.36 (m, 1H), 1.53 (t, J=12.6 Hz, 3H), 1.46 (d, J=11.2 Hz, 3H).

### Experimental Protocol for Compound 37

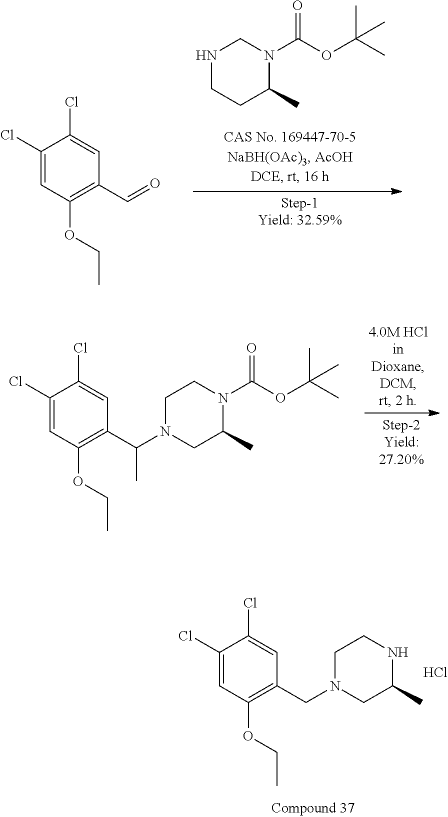 [SMILES: CCOc1cc(Cl)c(Cl)cc1C=O ; C[C@H]1CCNCN1C(=O)OC(C)(C)C ; CCOc1cc(Cl)c(Cl)cc1C(C)N1CCN(C(=O)OC(C)(C)C)[C@@H](C)C1 ; CCOc1cc(Cl)c(Cl)cc1CN1CCN[C@@H](C)C1 ; Cl]

### Step-1: Synthesis of tert-butyl (S)-4-(4,5-dichloro-2-ethoxybenzyl)-2-methylpiperazine-1-carboxylate

### Procedure

To a stirred solution of 4,5-dichloro-2-ethoxybenzaldehyde (0.5 g, 2.28 mmol) in DCE (10 mL) at room temperature, tert-butyl (S)-2-methylpiperazine-1-carboxylate (0.548 g, 2.73 mmol) and acetic acid (0.02 mL, 0.05V) were added. The reaction mixture was stirred at room temperature for 2 h. Then sodium triacetoxyborohydride (1.4 g, 6.84 mmol) was added portion-wise into reaction mixture at 0° C. The reaction was stirred at room temperature for 16 h. The progress of the reaction was monitored by TLC and LCMS analysis. After completion of the reaction, the reaction mixture was poured into a saturated sodium bicarbonate solution (100 mL) and extracted with CH₂Cl₂ (3×30 mL). The combined organic fractions were washed with water (2×20 mL), dried over sodium sulphate and concentrated under reduced pressure. The crude material was purified by column chromatography (neutral Alumina; 30-35% ethyl acetate in hexane) to provide tert-butyl (S)-4-(4,5-dichloro-2-ethoxybenzyl)-2-methylpiperazine-1-carboxylate (0.3 g, 32.59% yield) as a light yellow liquid.

LCMS [ESI, M+1]: 403.0 (RT: 1.769 min, Purity: 100%)

### Step-2: Synthesis of (S)-1-(4,5-dichloro-2-ethoxybenzyl)-3-methylpiperazine hydrochloride (Compound 37)

### Procedure

To a stirred solution of tert-butyl (S)-4-(4,5-dichloro-2-ethoxybenzyl)-2-methylpiperazine-1-carboxylate (0.3 g, 0.74 mmol) in CH₂Cl₂ (3 mL) at 0° C., 4M HCl in dioxane (1.5 mL, 5V) was added. The reaction mixture was stirred at room temperature for 2 h. The progress of the reaction was monitored by TLC and LCMS analysis. After completion of the reaction, the reaction mixture was concentrated under reduced pressure and the crude material purified by preparative HPLC (0.05% HCl in water/acetonitrile) to provide (S)-1-(4,5-dichloro-2-ethoxybenzyl)-3-methylpiperazine hydrochloride (0.068 g, 27.20% yield) as a white solid.

LCMS [ESI, M+1]: 302.8 (RT: 1.297 min, Purity: 100%),

HPLC: RT: 4.533 min, Purity: 97.16%,

Chiral HPLC: RT: 2.20 min, Purity: 100%,

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: METHANOL with 70-30 composition method; with Flow rate=3 ml/min; Column oven temperature 40° C.; ABPR 130 bar, analysis time 5 min.

¹H NMR (400 MHz, CD₃OD): δ 7.78 (s, 1H), 7.37 (s, 1H), 4.44 (s, 2H), 4.30-4.17 (d, J=11.9 Hz, 2H), 3.83 (s, 1H), 3.73 (d, J=10.7 Hz, 3H), 3.55 (dd, J=22.9, 10.7 Hz, 1H), 3.43 (d, J=11.9 Hz, 1H), 3.31-3.20 (m, 1H), 1.50 (t, J=11.9 Hz, 3H), 1.45 (d, J=6.5 Hz, 3H).

### Experimental Protocol for Compound 38

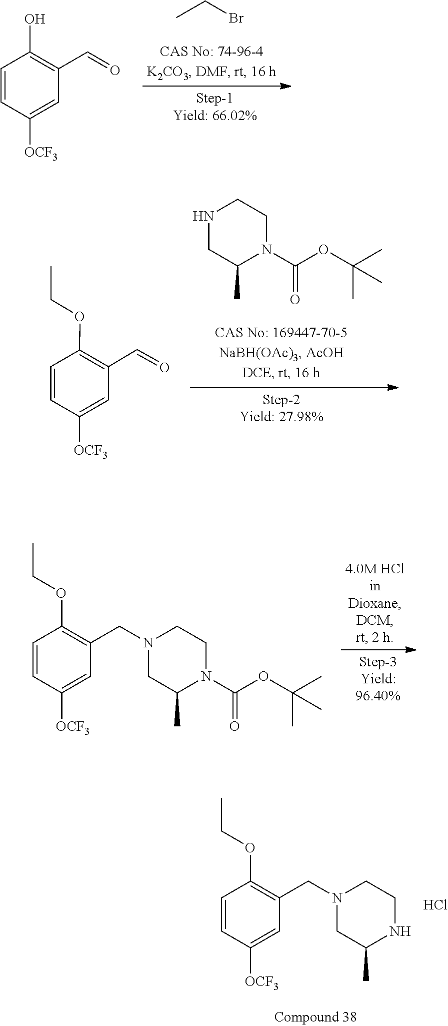

### Step-1: Synthesis of 2-ethoxy-5-(trifluoromethoxy)benzaldehyde

### Procedure

To a stirred solution of 2-hydroxy-5-(trifluoromethoxy)benzaldehyde (0.4 g, 1.941 mmol) in DMF (4 mL) at room temperature, K₂CO₃ (0.804 g, 5.82 mmol) was added. The reaction mixture was stirred for 1 h. Then bromoethane (0.314 g, 2.912 mmol) was added to the reaction mixture. The reaction mixture was stirred at room temperature for 16 h. The progress of the reaction was monitored by TLC and LCMS analysis. After completion of the reaction, the reaction mixture was poured into ice-cold water (40 mL) and extracted with ethyl acetate (3×20 mL). The combined organic fractions were dried over sodium sulphate and concentrated under reduced pressure. The crude material was purified by column chromatography (SiO₂; 21% ethyl acetate in hexane) to provide 2-ethoxy-5-(trifluoromethoxy)benzaldehyde (0.3 g, 66.02% yield) as an off-white sticky liquid.

¹H NMR (400 MHz, d₆-DMSO): δ 10.22 (s, 1H), 7.53 (s, 1H), 7.52 (s 1H), 7.50 (d, J=6.9 Hz, 1H), 4.14-4.11 (q, J=12.6 Hz, 2H) 1.34-1.31 (t, J=11.9 Hz, 3H)

### Step-2: Synthesis of tert-butyl (S)-4-(2-ethoxy-5-(trifluoromethoxy)benzyl)-2-methylpiperazine-1-carboxylate

### Procedure

To a stirred solution of 2-ethoxy-5-(trifluoromethoxy)benzaldehyde (0.3 g, 0.857 mmol) in DCE (3.0 mL) at room temperature, tert-butyl (S)-2-methylpiperazine-1-carboxylate (0.308 g, 1.538 mmol) and acetic acid (0.01 mL, 0.05V) were added. The reaction mixture was stirred at room temperature for 4 h. Sodium triacetoxyborohydride (0.811 g, 3.846 mmol) was added to the reaction mixture portion-wise at 0° C. The reaction was stirred at room temperature for 12 h. The progress of the reaction was monitored by TLC and LCMS analysis. After completion of reaction, the reaction mixture was poured into a saturated solution of sodium bicarbonate (30 mL) and extracted with CH₂Cl₂ (3×25 ml). The combined organic fractions were washed with water (20 mL), dried over sodium sulphate and concentrated under reduced pressure. The crude material was purified by column chromatography (SiO₂; eluted in 60-65% ethyl acetate in hexane) to provide tert-butyl (S)-4-(2-ethoxy-5-(trifluoromethoxy)benzyl)-2-methylpiperazine-1-carboxylate (0.15 g, 27.98% yield) as a colorless liquid.

LCMS [ESI, M+1]: 419.3 (RT: 1.632 min, Purity: 96.47%)

### Step 3: Synthesis of (S)-1-(2-ethoxy-5-(trifluoromethoxy)benzyl)-3-methylpiperazine hydrochloride (Compound 38)

### Procedure

To a stirred solution of tert-butyl (S)-4-(2-ethoxy-5-(trifluoromethoxy)benzyl)-2-methylpiperazine-1-carboxylate (0.15 g, 0.358 mmol) in CH₂Cl₂ (1.5 mL, 10V) at 0° C., 4M HCl in dioxane (0.7 mL, 5V) was added. The reaction mixture was stirred at room temperature for 2 h. The progress of the reaction was monitored by TLC and LCMS analysis. After completion of the reaction, the reaction mixture was concentrated under reduced pressure and the crude material triturated with diethyl ether (2×15 mL) to provide (S)-1-(2-ethoxy-5-(trifluoromethoxy)benzyl)-3-methylpiperazine hydrochloride (0.11 g, 96.40%) as a yellow solid.

LCMS [ESI, M+1]: 319.0 (RT: 1.193 min, Purity: 100%),

Chiral HPLC: 3.18 min, Purity: 99.45%

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% METHANOLIC AMMONIA in METHANOL with 90-10 composition method; with Flow rate=3 ml/min; Column oven temperature 40° C.; ABPR 130 bar, analysis time 6 min.

HPLC: RT: 4.433 min, Purity: 98.40%

¹H NMR (400 MHz, CD₃OD) δ 7.56 (t, J=11.1 Hz, 1H), 7.43 (d, J=7.0 Hz, 1H), 7.21 (d, J=9.1 Hz, 1H), 4.47 (d, J=19.9 Hz, 2H), 4.22 (dd, J=13.9, 7.0 Hz, 2H), 3.80 (d, J=25.0 Hz, 1H), 3.68 (dt, J=48.6, 13.7 Hz, 4H), 3.50-3.32 (m, 2H), 1.45 (dt, J=36.5, 10.3 Hz, 6H).

### Experimental Protocol for Compound 39

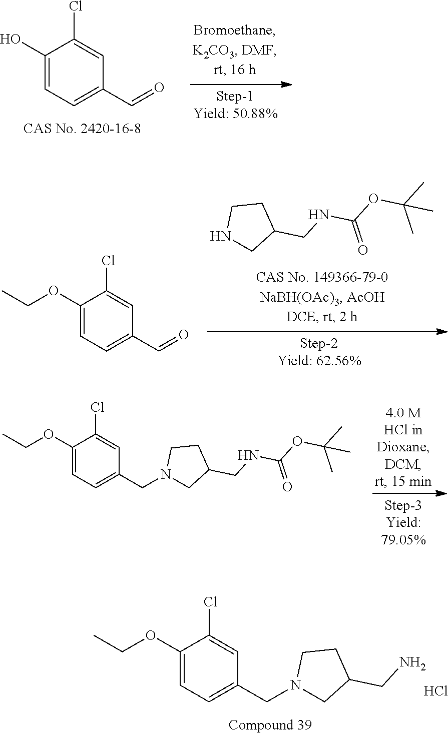

### Step-1: Synthesis of 3-chloro-4-ethoxybenzaldehyde

### Procedure

To a stirred solution of 3-chloro-4-hydroxybenzaldehyde (2.0 g, 12.77 mmol) in DMF (20 mL, 10V) at room temperature, potassium carbonate (5.28 g, 38.32 mmol) was added. The reaction mixture was stirred at room temperature for 30 min. Bromoethane (2.08 g, 19.16 mmol) was added at 0° C. The reaction mixture was stirred at room temperature for 16 h. The progress of the reaction was monitored by TLC and LCMS analysis. After completion of the reaction, the reaction mixture was poured into ice cold water (100 mL). A precipitate formed which was isolated by filtration and dried under reduced pressure to provide 3-chloro-4-ethoxybenzaldehyde (1.2 g, 50.88% yield) as a light yellow liquid.

¹H NMR (400 MHz, d₆-DMSO): δ 9.86 (s, 1H), 7.95 (d, J=2 Hz, 1H), 7.88 (dd, J=8 Hz, 4 Hz 1H), 7.35 (d, J=8H, 1H), 4.28-4.23 (q, 2H), 1.39 (t, 3H).

### Step-2: Synthesis of tert-butyl ((1-(3-chloro-4-ethoxybenzyl)pyrrolidin-3-yl)methyl)carbamate

### Procedure

To a stirred solution of 3-chloro-4-ethoxybenzaldehyde (0.2 g, 1.08 mmol) in DCE (2 mL, 10V) at room temperature, tert-butyl (pyrrolidin-3-ylmethyl)carbamate (0.26 g, 1.29 mmol) and acetic acid (0.01 mL, 0.05V) were added. The reaction mixture was stirred at room temperature for 2 h. Sodium triacetoxyborohydride (0.68 g, 3.87 mmol) was added to the reaction mixture portion-wise at 0° C. The reaction was stirred at room temperature for 2 h. The progress of the reaction was monitored by TLC and LCMS analysis. After completion of the reaction, the reaction mixture was poured into a saturated solution of sodium bicarbonate (30 mL) and extracted with CH₂Cl₂ (3×20 mL). The combined organic fractions were washed with water (20 mL), dried over sodium sulphate and concentrated under reduced pressure. The crude material was purified by trituration using n-pentane (3×20 mL) to provide tert-butyl ((1-(3-chloro-4-ethoxybenzyl)pyrrolidin-3-yl)methyl)carbamate (0.25 g, 62.56% yield) as a pale yellow liquid.

LCMS [ESI, M+1]: 368.95 (RT: 1.443 min, Purity: 95.19%).

### Step-3: Synthesis of (1-(3-chloro-4-ethoxybenzyl)pyrrolidin-3-yl)methanamine hydrochloride (Compound 39)

### Procedure

To a stirred solution of tert-butyl ((1-(3-chloro-4-ethoxybenzyl)pyrrolidin-3-yl)methyl)carbamate (0.25 g, 0.67 mmol) in CH₂Cl₂ (2.5 mL, 10V) at room temperature. 4M HCl in dioxane (1.25 mL, 5V) was added. The reaction mixture was stirred at room temperature for 15 min. The progress of the reaction was monitored by TLC and LCMS analysis. After completion of the reaction, the reaction mixture was concentrated under reduced pressure and the crude material purified by trituration using CH₂Cl₂ (2×15 mL) to provide (1-(3-chloro-4-ethoxybenzyl)pyrrolidin-3-Yl)methanamine hydrochloride (0.14 g, 79.05% yield) as a pale yellow solid.

LCMS [ESI, M+1]: 268.9 (RT: 0.794 min, Purity: 99.26%),

HPLC: RT: 4.100, Purity: 98.53% ¹H NMR (400 MHz, CD₃OD): δ 7.66-7.64 (m, 1H), 7.50 (dd, J=8.0, 6.3 Hz, 1H), 7.18 (t, J=11.0 Hz, 1H), 4.48-4.28 (m, 2H), 4.19 (q, J=4.9 Hz, 2H), 3.65 (m, 2H), 3.55-3.44 (m, 1H), 3.44-3.35 (m, 1H), 3.16 (t, J=7.4 Hz, 1H), 3.11-2.98 (m, 1H), 2.83 (m, 1H), 2.53-2.25 (m, 1H), 2.10-1.79 (m, 1H), 1.50-1.43 (t, J=11.9 Hz, 3H).

### Experimental Protocol for Compound 40

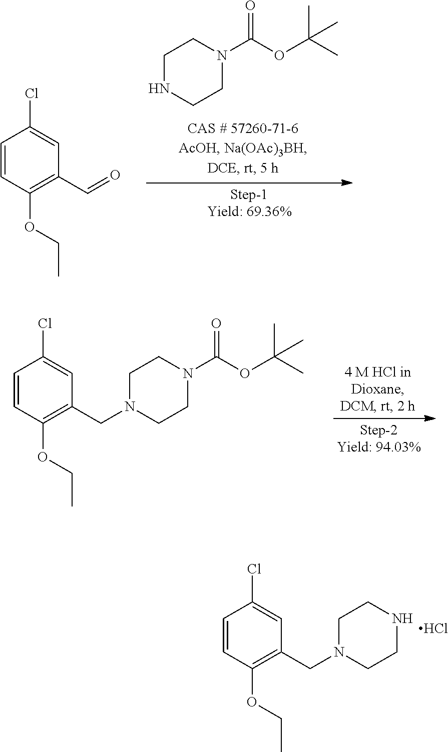 [SMILES: CCOc1ccc(Cl)cc1C=O ; CC(C)(C)OC(=O)N1CCNCC1 ; CCOc1ccc(Cl)cc1CN1CCN(C(=O)OC(C)(C)C)CC1 ; CCOc1ccc(Cl)cc1CN1CCNCC1 ; Cl]

### Step-1: Synthesis of tert-butyl 4-(5-chloro-2-ethoxybenzyl)piperazine-1-carboxylate

### Procedure

To a stirred solution of 5-chloro-2-ethoxybenzaldehyde (0.3 g, 1.625 mmol) in DCE (5 mL) at room temperature, tert-butyl piperazine-1-carboxylate (0.363 g, 1.94 mmol) and acetic acid (0.009 g, 0.1625 mmol) were added. The reaction mixture was stirred at room temperature for 2 h. Sodium triacetoxyborohydride (1.033 g, 4.874 mmol) was added portion-wise to the reaction mixture at 0° C. The reaction mixture was allowed to stir at room temperature for 5 h. The progress of the reaction was monitored by TLC. After completion of the reaction, the reaction mixture was diluted with water (10 mL) and extracted in CH₂Cl₂ (3×10 mL). The combined organic fractions were washed with a sat. NaHCO₃ solution (2×20 mL), dried over anhydrous sodium sulphate and concentrated under reduced pressure to provide tert-butyl 4-(5-chloro-2-ethoxybenzyl)piperazine-1-carboxylate (0.4 g, 69.36% yield) as a colorless sticky solid.

LCMS [ESI, M+1]: 355 (RT: 1.488 min, Purity: 97.08%).

### Step-2: Synthesis of 1-(5-chloro-2-ethoxybenzyl)piperazine hydrochloride (Compound 40)

### Procedure

To a stirred solution of tert-butyl 4-(5-chloro-2-ethoxybenzyl) piperazine-1-carboxylate (0.4 g, 1.127 mmol) in CH₂Cl₂ (4 mL) at 0° C., 4.0 M HCl in dioxane (2 mL, 5V) was added.

The reaction mixture was stirred at room temperature for 2 h. The progress of the reaction was monitored by TLC. After completion of reaction, the reaction mixture was concentrated under reduced pressure and the crude material purified by trituration using diethyl ether (2×5 mL) to provide 1-(5-chloro-2-ethoxybenzyl)piperazine hydrochloride (0.28 g, 94.03% yield) as a white solid.

LCMS [ESI, M+1]: 254.93 (RT: 0.894 min, Purity: 99.18%),

HPLC Purity: RT: 4.040, Purity: 99.62% ¹H NMR (400 MHz, d₆-DMSO): 9.65 (s, 2H), 7.70 (s, 1H), 7.49 (d, J=6.8 Hz, 1H), 7.14 (d, J=8.9 Hz, 1H), 4.27 (s, 2H), 4.10 (q, J=6.9 Hz, 2H), 3.34 (m, 8H), 1.42-1.33 (m, 3H).

### Experimental Protocol for Compound 41

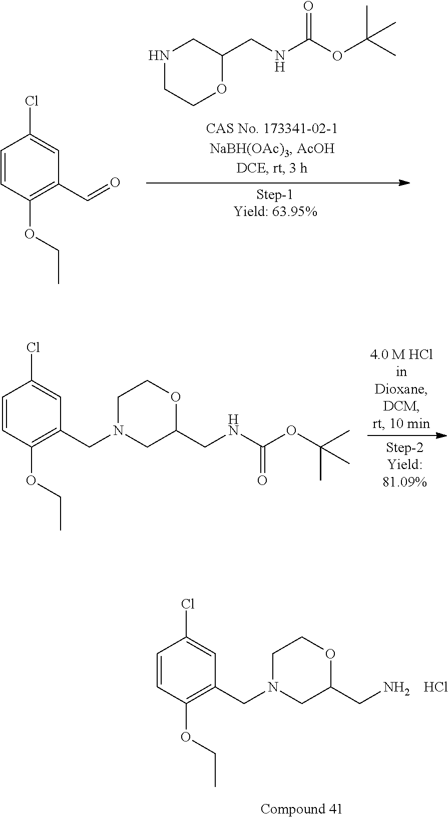 [SMILES: CCOc1ccc(Cl)cc1C=O ; CC(C)(C)OC(=O)NCC1CNCCO1 ; CCOc1ccc(Cl)cc1CN1CCOC(CNC(=O)OC(C)(C)C)C1 ; CCOc1ccc(Cl)cc1CN1CCOC(CN)C1 ; Cl]

### Step-1: Synthesis of tert-butyl ((4-(5-chloro-2-ethoxybenzyl)morpholin-2-yl)methyl)carbamate

### Procedure

To a stirred solution of 5-chloro-2-ethoxybenzaldehyde (0.15 g, 0.81 mmol) in DCE (1.5 mL, 10V) at room temperature, tert-butyl (morpholin-2-ylmethyl)carbamate (0.21 g, 0.97 mmol) and acetic acid (0.002 g, 0.0408 mmol) were added. The reaction mixture was stirred at room temperature for 2 h. Sodium triacetoxyborohydride (0.518 g, 2.44 mmol) was added to the reaction mixture portion-wise at 0° C. The reaction was stirred at room temperature for 1 h. The progress of the reaction was monitored by TLC and LCMS analysis. After completion of the reaction, the reaction mixture was poured into a saturated solution of sodium bicarbonate (30 mL) and extracted with CH₂Cl₂ (3×20 mL). The combined organic fractions were washed with water (20 mL), dried over sodium sulphate and concentrated under reduced pressure. The crude material was purified by trituration using n-Pentane (3×10 mL) to provide tert-butyl ((4-(5-chloro-2-ethoxybenzyl)morpholin-2-yl)methyl)carbamate (0.122, 63.95% yield) as a pale yellow sticky solid.

LCMS [ESI, M+1]: 385.01 (RT: 1.482 min, Purity: 99.16%).

### Step-2: Synthesis of (4-(5-chloro-2-ethoxybenzyl)morpholin-2-Yl)methanamine hydrochloride (Compound 41)

### Procedure

To a stirred solution of tert-butyl ((4-(5-chloro-2-ethoxybenzyl)morpholin-2-yl)methyl)carbamate (0.2 g, 0.519 mmol) in CH₂Cl₂ (2 mL, 10V) at 0° C., 4M HCl in dioxane (1 mL, 5V) was added. The reaction mixture was stirred at room temperature for 10 min. The progress of the reaction was monitored by TLC and LCMS analysis. After completion of the reaction, the reaction mixture was concentrated under reduced pressure and the crude material purified by trituration using CH₂Cl₂ (2×10 mL) to provide (4-(5-chloro-2-ethoxybenzyl)morpholin-2-yl)methanamine hydrochloride (0.1222, 81.09% yield) as a brown solid.

LCMS [ESI, M+1]: 284.89, (RT: 0.783 min, Purity: 100%)

HPLC: RT: 3.980, Purity: 100% ¹H NMR (400 MHz, CD₃OD): δ 7.62 (d, J=2.6 Hz, 1H), 7.50 (dd, J=8.9, 2.6 Hz, 1H), 7.15 (d, J=8.9 Hz, 1H), 4.48-4.35 (m, 2H), 4.26 (q, J=12.6 Hz, 4H), 4.02-3.91 (m, 1H), 3.63-3.55 (m, 1H), 3.54-3.46 (m, 1H), 3.30-3.21 (m, 2H), 3.05 (m, 2H), 1.50 (t, J=7.0 Hz, 3H).

### Experimental Protocol for Compound 42

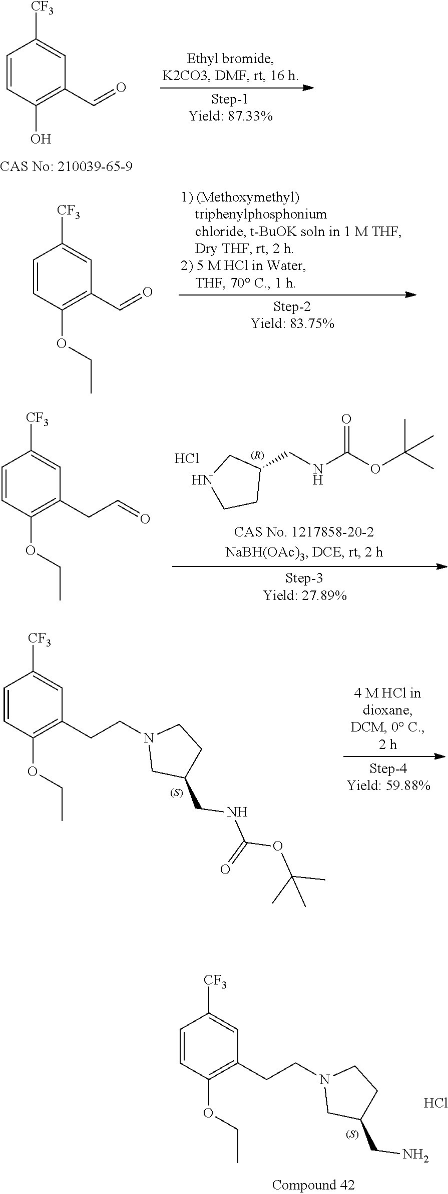

### Step-1: Synthesis of 2-ethoxy-5-(trifluoromethyl)benzaldehyde

### Procedure

To a stirred solution of 2-hydroxy-5-(trifluoromethyl)benzaldehyde (2.0 g, 10.52 mmol) in DMF (20 mL) at room temperature, potassium carbonate (3.6 g, 26.30 mmol) was added. The reaction mixture was stirred at room temperature for 1 h. Ethyl bromide (2.3 g, 21.04 mmol) was added to the reaction mixture at room temperature. The reaction mixture was stirred at room temperature for 16 h. The progress of the reaction was monitored by TLC. After completion of the reaction, the reaction mixture was poured into ice cold water (100 mL) and extracted with ethyl acetate (3×40 mL). The combined organic fractions were dried over sodium sulphate and concentrated under reduced pressure to provide 2-ethoxy-5-(trifluoromethyl)benzaldehyde (2.2 g, 87.33%) as a light yellow liquid.

LCMS [ESI, M+1]: 218.7 (RT: 2.103 min, Purity: 100%),

¹H NMR (400 MHz, CDCl3) δ 10.49 (s, 1H), 8.12 (d, J=2.2 Hz, 1H), 7.79 (dd, J=8.8, 2.0 Hz, 1H), 7.08 (s, 1H), 4.24 (t, J=7.0 Hz, 2H), 1.54 (t, J=7.0 Hz, 3H).

### Step-2: 2-(2-ethoxy-5-(trifluoromethyl)phenyl)acetaldehyde

### Procedure

To a stirred solution of (Methoxymethyl)triphenyl phosphine (2.1 g, 6.29 mmol) in anhydrous THF (20 mL) at 0° C., t-BuOK in 1M THF (12.5 mL, 12.58 mmol) was added. The reaction mixture was stirred at room temperature for 1 h. Then 2-ethoxy-5-(trifluoromethyl)benzaldehyde (0.5 g, 2.29 mmol) in anhydrous THF (10 mL) was added drop-wise into the reaction mixture at room temperature. The reaction mixture was stirred at room temperature for 1 h. The progress of the reaction was monitored by TLC. After completion of the reaction, the reaction mixture was concentrated under reduced pressure and the crude material purified by flash column chromatography (Aluminum oxide neutral; 0-2% ethyl acetate in hexane) to provide (E)-1-ethoxy-2-(3-methoxyallyl)-4-(trifluoromethyl)benzene (0.5 g, 83.75%) as a colorless oil.

To a stirred solution of (E)-1-ethoxy-2-(3-methoxyallyl)-4-(trifluoromethyl)benzene (0.4 g, 1.49 mmol) in THF (6 mL) at room temperature, 5M HCl (6 mL) was added. The reaction mixture was stirred at 70° C. for 1 h. The progress of the reaction was monitored by TLC. After completion of the reaction, the reaction mixture was quenched with a saturated solution of sodium bicarbonate (50 mL) and extracted with ethyl acetate (2×100 mL). The combined organic fractions were dried over sodium sulphate and concentrated under reduced pressure to provide 2-(2-ethoxy-5-(trifluoromethyl)phenyl)acetaldehyde (0.5 g, 83.75%) as a light yellow liquid.

### Step-3: Synthesis of tert-butyl (S)-((1-(2-ethoxy-5-(trifluoromethyl)phenethyl)pyrrolidin-3-yl)methyl) carbamate

### Procedure

To a stirred solution of 2-(2-ethoxy-5-(trifluoromethyl)phenyl)acetaldehyde (0.5 g, 1.29 mmol) in DCE (5 mL) at room temperature, tert-butyl (R)-(pyrrolidin-3-ylmethyl)carbamate hydrochloride (0.56 g, 2.36 mmol) was added. The reaction mixture was stirred at room temperature for 1 h. Sodium triacetoxyborohydride (1.14 g, 5.38 mmol) was added portion-wise to the reaction mixture at 0° C. The reaction was stirred at room temperature for 1 h. The progress of the reaction was monitored by TLC. After completion of the reaction, the reaction mixture was poured into a saturated solution of sodium bicarbonate (50 mL) and extracted with CH₂Cl₂ (2×40 mL). The combined organic fractions were washed with water (30 mL), dried over sodium sulphate and concentrated under reduced pressure. The crude material was purified by column chromatography (Aluminum oxide neutral; 30-35% ethyl acetate in hexane) to provide tert-butyl (S)-((1-(2-ethoxy-5-(trifluoromethyl)phenethyl)pyrrolidin-3-yl)methyl)carbamate (0.25 g, 27.89% yield) as a light yellow liquid.

LCMS [ESI, M+1]: 417.0 (RT: 1.726 min, Purity: 67.57%).

### Step-4: Synthesis of (S)-(1-(2-ethoxy-5-(trifluoromethyl)phenethyl)pyrrolidin-3-yl)methanamine hydrochloride (Compound 42)

### Procedure

To a stirred solution of tert-butyl (S)-((1-(2-ethoxy-5-(trifluoromethyl)phenethyl)pyrrolidin-3-yl)methyl)carbamate (0.22 g, 0.52 mmol) in CH₂Cl₂ (2.2 mL) at 0° C., 4M HCl in dioxane (0.9 mL, 4V) was added. The reaction mixture was stirred at 0° C. for 2 h. The progress of the reaction was monitored by TLC and LCMS analysis. After completion of the reaction, the reaction mixture was concentrated under reduced pressure and the crude material was purified by reverse phase column chromatography (C18 silica as stationary phase; 0.05% HCl in water/acetonitrile) to provide (S)-(1-(2-ethoxy-5-(trifluoromethyl)phenethyl)pyrrolidin-3-yl)methanamine hydrochloride (0.1 g, 59.88% yield) as a yellow liquid.

LCMS [ESI, M+1]: 316.8 (RT: 0.989 min, Purity: 97.89%),

HPLC: RT: 4.580 min, Purity: 95.09%,

Chiral HPLC: RT: 1.99 min, Purity: 100%,

Instrument Name: Waters SFC Investigator

Chromatographic separation was conducted with Waters SFC Investigator system with 2998 PDA detector. The column used was CHIRALCEL® ODH (250×4.6 mm; 5 ␣m) and the compounds were eluted with, Mobile Phase A: Liq·CO2, Mobile Phase B: 0.1% METHANOLIC AMMONIA in METHANOL with 85-15 composition method; with Flow rate=5 ml/min; Column oven temperature 40° C.; ABPR 130 bar, analysis time 5 min.

¹H NMR (400 MHz, CD₃OD) δ 7.61 (d, J=4.1 Hz, 1H), 7.60 (s, 1H), 7.20-7.10 (m, 1H), 4.22 (q, J=7.0 Hz, 2H), 3.83 (s, 2H), 3.67-3.42 (m, 4H), 3.23-3.11 (m, 4H), 2.85 (s, 1H), 2.42 (s, 1H), 1.98 (d, J=21.8 Hz, 1H), 1.51 (t, J=7.0 Hz, 3H).

### Experimental Protocol for Compound 43

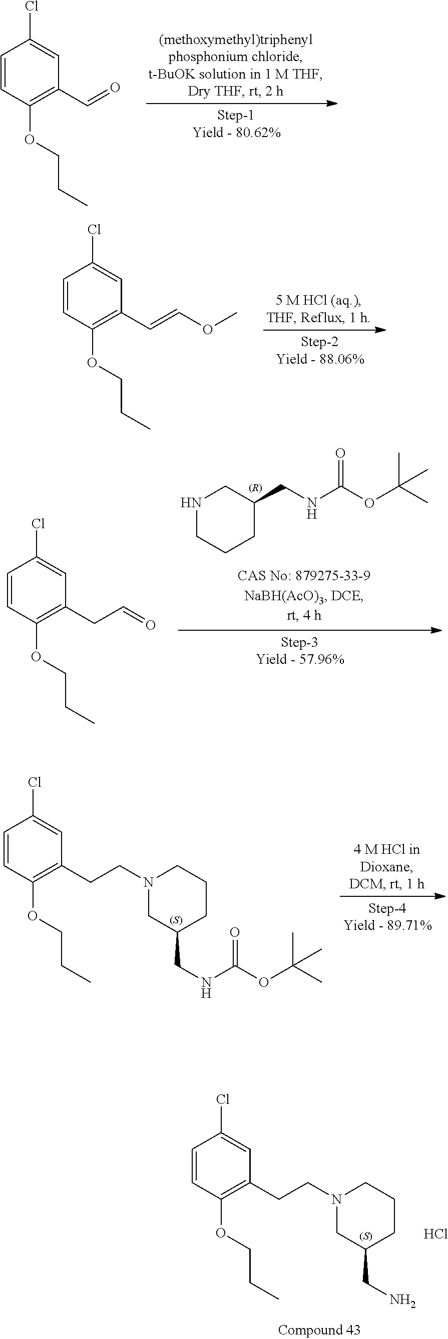

### Step-1: Synthesis of (E)-4-chloro-2-(2-methoxyvinyl)-1-propoxybenzene

### Procedure

To a stirred solution of (methoxymethyl)triphenyl phosphonium chloride (2.16 g, 6.29 mmol) in anhydrous THF (20 mL, 40V) at 0° C., t-BuOK solution in 1M THF (12.5 mL, 25.25 mmol) was added. The reaction mixture was stirred at room temperature for 1 h. 5-chloro-2-propoxybenzaldehyde (0.5 g, 2.51 mmol) dissolved in THF (1 mL) was added drop-wise to the reaction mixture at 0° C. The reaction mixture was stirred at room temperature for 1 h. The progress of the reaction was monitored by TLC. After completion of the reaction, the reaction mixture was concentrated under reduced pressure and the crude material purified by column chromatography (neutral alumina; 100% hexane) to provide (E)-4-chloro-2-(2-methoxyvinyl)-1-propoxybenzene (0.46 g, 80.62% yield) as a light yellow liquid.

### Step-2: Synthesis of 2-(5-chloro-2-propoxyphenyl)acetaldehyde

### Procedure

To a stirred solution of (E)-4-chloro-2-(2-methoxyvinyl)-1-propoxybenzene (0.46 g, 2.03 mmol) in anhydrous THF (4.6 mL, 10V) at room temperature, 5M HCl solution (2.3 mL, 5V) was added. The reaction mixture was heated at reflux for 1 h. The progress of the reaction was monitored by TLC analysis. After completion of the reaction, the reaction mixture was poured into a saturated solution of sodium bicarbonate (40 mL) and extracted with ethyl acetate (3×20 mL). The combined organic fractions were washed with water (20 mL), dried over sodium sulphate and concentrated under reduced pressure to provide 2-(5-chloro-2-propoxyphenyl) acetaldehyde (0.382, 88.06% yield) as an off-white sticky solid (confirmed by TLC analysis with the 2.4-DNP stain).

### Step-3: Synthesis of tert-butyl (S)-((1-(5-chloro-2-propoxyphenethyl)piperidin-3-yl)methyl)carbamate

### Procedure

To a stirred solution of 2-(5-chloro-2-propoxyphenyl)acetaldehyde (0.25 g, 1.17 mmol) in DCE (5 mL, 20V) at room temperature, tert-butyl (R)-(piperidin-3-ylmethyl)carbamate (0.30 g, 1.41 mmol) and Acetic acid (0.003 mL, 0.05V) were added. The reaction mixture was stirred at room temperature for 1 h. Sodium triacetoxyborohydride (0.75 g, 3.53 mmol) was added portion-wise to the reaction mixture at 0° C. The reaction was stirred at room temperature for 3 h. The progress of the reaction was monitored by TLC and LCMS analysis. After completion of the reaction, the reaction mixture was poured into a saturated solution of sodium bicarbonate (30 mL) and extracted with CH₂Cl₂ (3×15 mL). The combined organic fractions were washed with water (25 mL), dried over sodium sulphate and concentrated under reduced pressure. The crude material was purified by column chromatography (neutral alumina; 35% ethyl acetate in hexane) to provide tert-butyl (S)-((1-(5-chloro-2-propoxyphenethyl)piperidin-3-yl)methyl)carbamate (0.282, 57.96% yield) as a light yellow liquid.

LCMS [ESI, M+1]: 411.11 (RT: 1.772 min, Purity: 95.91%).

### Step-4: Synthesis of (S)-(1-(5-chloro-2-propoxyphenethyl)piperidin-3-yl)methanamine hydrochloride (Compound 43)

### Procedure

To a stirred solution of tert-butyl (S)-((1-(5-chloro-2-propoxyphenethyl)piperidin-3-yl)methyl)carbamate (0.28 g, 0.68 mmol) in CH₂Cl₂ (2.8 mL, 10V) at 0° C., 4M HCl in dioxane (1.4 mL, 5.0V) was added. The reaction mixture was stirred at room temperature for 1 h. After completion of the reaction, the reaction mixture was concentrated under reduced pressure and the crude material purified by trituration with diethyl ether (2×20 mL) to provide (S)-(1-(5-chloro-2-propoxyphenethyl)piperidin-3-yl)methanamine hydrochloride (0.192, 89.71% yield) as an off-white solid.

LCMS [ESI, M+1]: 310.89 (RT: 1.119 min, Purity: 100),

HPLC: RT: 4.660 min, Purity: 98.15%,

Chiral HPLC: RT: 2.42 min, Purity: 99.54%,

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% METHANOLIC AMMONIA in METHANOL with 75-25 composition method; with Flow rate=3 ml/min; Column oven temperature 40° C.; ABPR 130 bar, analysis time 5 min.

¹H NMR (400 MHz, CD₃OD): δ 7.30 (d, J=2.8 Hz, 1H), 7.27-7.25 (dd, J=4.9 Hz 1H), 6.98 (d, J=12 Hz 1H), 4.01 (t, J=12 Hz, 2H), 3.74-3.68 (m, 2H), 3.30 (s, 1H), 3.16-3.12 (m, 2H), 3.05-30.00 (m, 2H), 2.96-2.86 (m, 2H), 2.36 (m, 1H), 2.03 (m, 2H), 1.93-1.86 (m, 3H), 1.38-1.35 (m, 2H), 1.10 (t, J=16 Hz, 3H).

### Experimental Protocol for Compound 44

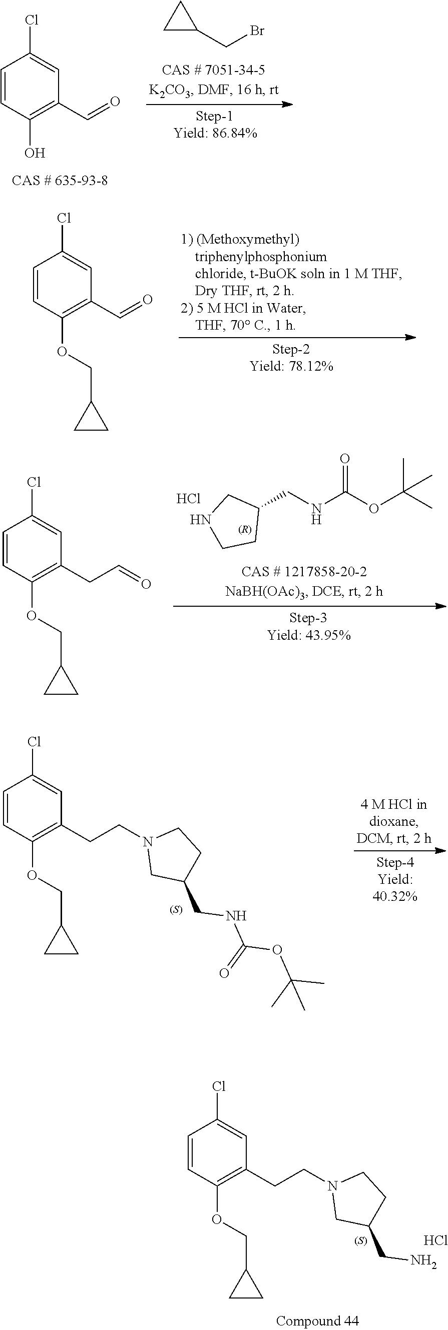

### Step-1: Synthesis of 5-chloro-2-(cyclopropylmethoxy)benzaldehyde

### Procedure

To a stirred solution of 5-chloro-2-hydroxybenzaldehyde (3.0 g, 19 mmol) in DMF (6 mL) at room temperature, K₂CO₃ (7.9 g, 57.4 mmol) was added. The reaction was stirred for 1 h. Then (bromomethyl)cyclopropane (3.89 g, 28.2 mmol) was added to the reaction mixture. The reaction mixture was stirred at room temperature for 15 h. The progress of the reaction was monitored by TLC. After completion of reaction, the reaction mixture was poured into ice-cold water (15 mL) and extracted with ethyl acetate (3×10 mL). The combined organic fractions were dried over sodium sulphate and concentrated under reduced pressure. The crude material was purified by column chromatography (SiO₂; 10% EtOAc/hexane) to provide 5-chloro-2-(cyclopropylmethoxy)benzaldehyde (3.8 g, 93.82% yield) as a white solid.

¹H NMR (400 MHz, d₆-DMSO): δ 10.26 (s, 1H), 7.60 (dd, J=8.9, 2.8 Hz, 1H), 7.53 (d, J=2.8 Hz, 1H), 7.19 (d, J=9.0 Hz, 1H), 3.93 (d, J=7.0 Hz, 2H), 1.25-1.10 (m, 1H), 0.60-0.41 (m, 2H), 0.35-0.22 (m, 2H)

### Step-2: Synthesis of 2-(5-chloro-2-(cyclopropylmethoxy)phenyl)acetaldehyde

### Procedure

To a stirred solution of (Methoxymethyl)triphenyl phosphine (2.1 g, 6.16 mmol) in anhydrous THF (20 mL) at room temperature, t-BuOK in 1M THF (15.43 mL, 15.43 mmol) was added. The reaction mixture was stirred at room temperature for 1 h. 5-chloro-2-(cyclopropylmethoxy)benzaldehyde (0.6 g, 3.08 mmol) in anhydrous THF (10 mL) was added drop-wise into the reaction mixture at room temperature. The reaction mixture was stirred at room temperature for 2 h. The progress of the reaction was monitored by TLC. After completion of the reaction, the reaction mixture was concentrated under reduced pressure and the resulting crude material purified by column chromatography (Aluminium oxide neutral; 0-2% ethyl acetate in hexane) to provide (E)-4-chloro-1-(cyclopropylmethoxy)-2-(3-methoxyallyl)benzene (0.5 g, 69.25%) as a colorless oil.

To a stirred solution of (E)-4-chloro-1-(cyclopropylmethoxy)-2-(3-methoxyallyl)benzene (0.5 g, 1.98 mmol) in THF (6 mL) at room temperature, 5M HCl (6 mL) was added. The reaction mixture was stirred at 70° C. for 1 h. The progress of the reaction was monitored by TLC. After completion of the reaction, the reaction was quenched with a saturated solution of sodium bicarbonate (50 mL) and extracted with Ethyl acetate (2×100 mL). The combined organic fractions were dried over sodium sulphate and concentrated under reduced pressure to provide 2-(5-chloro-2-(cyclopropylmethoxy)phenyl)acetaldehyde (0.4 g, 78.12%) as a light yellow liquid.

### Step-3: Synthesis of tert-butyl (S)-((1-(5-chloro-2-(cyclopropylmethoxy)phenethyl)pyrrolidin-3-yl)methyl)carbamate

### Procedure

To a stirred solution of 2-(5-chloro-2-(cyclopropylmethoxy)phenyl)acetaldehyde (0.4 g, 1.98 mmol) in DCE (5 mL) at room temperature, tert-butyl (R)-(pyrrolidin-3-ylmethyl)carbamate hydrochloride (0.396 g, 1.98 mmol) was added. The reaction mixture was stirred at room temperature for 1 h. Sodium triacetoxyborohydride (0.839 g, 3.96 mmol) was added portion-wise to the reaction mixture at 0° C. The reaction was stirred at room temperature for 2 h. The progress of the reaction was monitored by TLC and LCMS analysis. After completion of the reaction, the reaction mixture was poured into a saturated solution of sodium bicarbonate (20 mL) and extracted with CH₂Cl₂ (2×100 mL). The combined organic fractions were was washed with water (50 mL), dried over sodium sulphate and concentrated under reduced pressure. The crude material was purified by column chromatography (Aluminum oxide neutral; 30-35% ethyl acetate in hexane) to provide tert-butyl (S)-((1-(5-chloro-2-(cyclopropylmethoxy)phenethyl)pyrrolidin-3-yl)methyl)carbamate (0.35 g, 43.95% yield) as a light yellow liquid.

LCMS [ESI, M+1]:409.6 (RT: 1.722 min, Purity: 74.29%).

### Step-4: Synthesis of (S)-(1-(5-chloro-2-(cyclopropylmethoxy)phenethyl)pyrrolidin-3-yl)methanamine hydrochloride (Compound 44)

### Procedure

To a stirred solution of tert-butyl (S)-((1-(5-chloro-2-(cyclopropylmethoxy)phenethyl)pyrrolidin-3-yl)methyl)carbamate (0.165 g, 0.40 mmol) in CH₂Cl₂ (0.5 mL) at 0° C., 4M HCl in dioxane (0.8 mL) was added. The reaction mixture was stirred at room temperature for 2 h. The progress of the reaction was monitored by TLC.

After completion of the reaction, the reaction mixture was concentrated under reduced pressure and the crude material purified by preparative HPLC (0.05% HCl in water/acetonitrile) to provide (S)-(1-(5-chloro-2-(cyclopropylmethoxy)phenethyl)pyrrolidin-3-yl)methanamine hydrochloride (0.05 g, 40.32% yield) as a yellow sticky solid.

LCMS [ESI, M+1]: 308.09 (RT: 1.062 min, Purity: 100%),

HPLC: RT: 4.420 min, Purity: 98.64%,

Chiral HPLC: RT: 2.23 min, Purity: 100%,

Instrument Name: Waters SFC Investigator

Chromatographic separation was conducted with Waters SFC Investigator system with 2998 PDA detector. The column used was CHIRALPAK® IG (250×4.6 mm; 5 ␣m) and the compounds were eluted with, Mobile Phase A: Liq·CO2, Mobile Phase B: 0.1% DIETHYLAMINE in METHANOL-ACETONITRILE (50-50) with 55-45 composition method; with Flow rate=3 ml/min; Column oven temperature 40° C.; ABPR 130 bar, analysis time 6 min.

¹H NMR (400 MHz, CD₃OD): δ 7.30 (d, J=5.2 Hz, 1H), 7.27-7.24 (m, 1H), 6.98 (d, J=5.2 Hz, 1H), 4.94 (d, J=7.0 Hz, 2H), 3.92-3.37 (m, 3H), 3.28-3.20 (m, 1H), 3.17 (m, 3H), 2.70 (m, 1H), 2.40 (m, 1H), 2.00 (m, 1H) 1.31 (m, 1H), 0.69 (m, 2H), 0.66 (m, 2H).

### Experimental Protocol for Compound 45

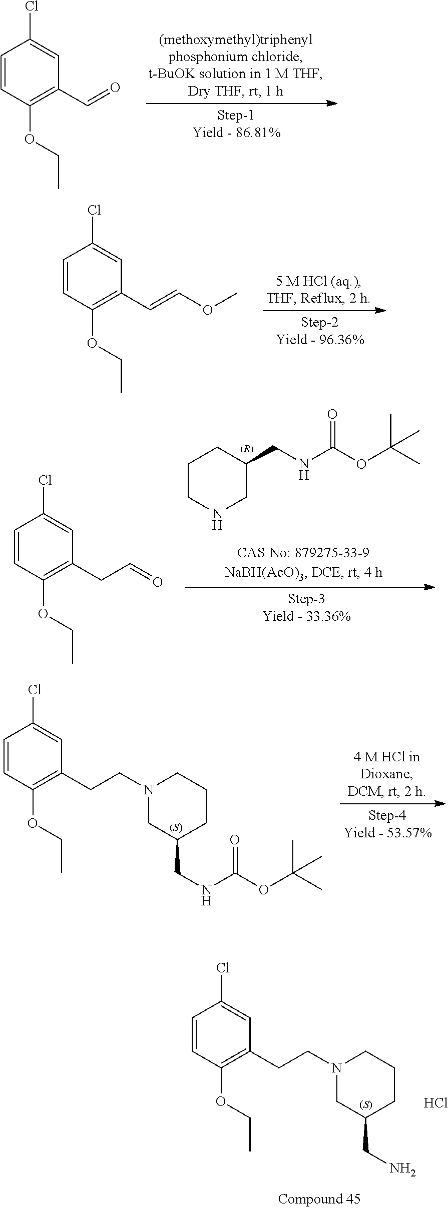

### Step-1: Synthesis of (E)-4-chloro-1-ethoxy-2-(2-methoxyvinyl)benzene

### Procedure

To a stirred solution of (methoxymethyl)triphenyl phosphonium chloride, (2.3 g, 6.77 mmol) in anhydrous THF (10 mL, 20V) at 0° C., t-BuOK solution in 1M THF (13.5 mL) was added. The reaction mixture was stirred at room temperature for 1 h. 5-chloro-2-ethoxybenzaldehyde (0.5 g, 2.71 mmol) dissolved in THF (3 mL) was added drop-wise to the reaction mixture at 0° C. The reaction was stirred at room temperature for 1 h. The progress of the reaction was monitored by TLC. After completion of reaction, the reaction mixture was concentrated under reduced pressure. The crude material was purified by column chromatography (neutral silica; 100% hexane) to provide (E)-4-chloro-1-ethoxy-2-(2-methoxyvinyl)benzene (0.5 g, 86.81% yield) as a light yellow liquid.

### Step-2: Synthesis of 2-(5-chloro-2-ethoxyphenyl)acetaldehyde (SLN5-X-0467-Int-A2)

### Procedure

To a stirred solution of (E)-4-chloro-1-ethoxy-2-(2-methoxyvinyl)benzene (0.5 g, 2.35 mmol) in anhydrous THF (5 mL, 10V) at 0° C., 5M HCl solution (2.5 mL, 5V) was added. The reaction was stirred at 70° C. for 2 h. The progress of the reaction was monitored by TLC. After completion of the reaction, the reaction mixture was poured into a saturated solution of sodium bicarbonate (50 mL) and extracted with ethyl acetate (2×25 mL). The combined organic fractions were washed with water (2×20 mL), dried over sodium sulphate and concentrated under reduced pressure to provide 2-(5-chloro-2-ethoxyphenyl)acetaldehyde (0.45 g, 96.36% yield) as a white sticky solid.

### Step-3: Synthesis of tert-butyl (S)-((1-(5-chloro-2-ethoxyphenethyl)piperidin-3-yl)methyl)carbamate

### Procedure

To a stirred solution of 2-(5-chloro-2-ethoxyphenyl)acetaldehyde (0.45 g, 2.27 mmol) in DCE (4.5 mL, 10V) at room temperature, tert-butyl (R)-(piperidin-3-ylmethyl)carbamate (0.582 g, 2.72 mmol) and Acetic acid (0.02 mL, 0.05V) were added. The reaction mixture was stirred at room temperature for 1 h. Sodium triacetoxyborohydride (1.43 g, 6.818 mmol) was added portion-wise to the reaction mixture at 0° C. The reaction was stirred at room temperature for 4 h. The progress of the reaction was monitored by TLC and LCMS analysis. After completion of the reaction, the reaction mixture was poured into a saturated solution of sodium bicarbonate (50 mL) and extracted with CH₂Cl₂ (3×20 mL). The combined organic fractions were washed with water (30 mL), dried over sodium sulphate and concentrated under reduced pressure. The crude material was purified by flash column chromatography (neutral silica; 20% ethyl acetate in hexane using) to provide tert-butyl (S)-((1-(5-chloro-2-ethoxyphenethyl)piperidin-3-yl)methyl)carbamate (0.3 g, 33.36% yield) as a light yellow liquid.

LCMS [ESI, M+100]: 397.1 (RT: 1.634 min, Purity: 91.34%).

### Step-4: Synthesis of (S)-(1-(5-chloro-2-ethoxyphenethyl)piperidin-3-Yl)methanamine hydrochloride (Compound 45)

### Procedure

To a stirred solution of tert-butyl (S)-((1-(5-chloro-2-ethoxyphenethyl)piperidin-3-yl)methyl)carbamate (0.3 g, 0.47 mmol) in CH₂Cl₂ (1.8 mL, 10V) at 0° C., 4M HCl in dioxane (0.5 mL, 3.0V) was added. The reaction mixture was stirred at room temperature for 2 h. After completion of the reaction, the reaction mixture was concentrated under reduced pressure and the crude material triturated with diethyl ether (2×20 mL) to provide (S)-(1-(5-chloro-2-ethoxyphenethyl)piperidin-3-yl)methanamine hydrochloride (0.12 g, 53.57% yield) as an off-white solid.

LCMS [ESI, M+1]: 296.9 (RT: 0.982 min, Purity: 96.95%),

HPLC: RT: 4.807 min, Purity: 95.07%,

Chiral HPLC: RT: 3.39 min, Purity: 95.24%,

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% DIETHYLAMINE in METHANOL with 80-20 composition method; with Flow rate=3 ml/min; Column oven temperature 40° C.; ABPR 130 bar, analysis time 7 min.

¹H NMR (400 MHz, CD₃OD) δ 7.31 (d, J=2.5 Hz, 1H), 7.26 (dd, J=8.7, 2.5 Hz, 1H), 6.99 (d, J=8.7 Hz, 1H), 4.12 (q, J=6.9 Hz, 2H), 3.9 (dd, J=19.4, 11.3 Hz, 1H), 3.85 (dd, J=19.9, 10.5 Hz, 1H), 3.60 (d, J=4.1 Hz, 1H), 3.45 (d, J=6.1 Hz, 2H), 3.29-3.14 (m, 2H), 3.16-3.08 (m, 2H), 3.00 (d, J=11.1 Hz, 1H), 2.78 (s, 1H), 2.56-2.24 (m, 1H), 2.07 (m, 3H), 1.48 (t, J=7.0 Hz, 3H).

### Experimental Protocol for Compound 46

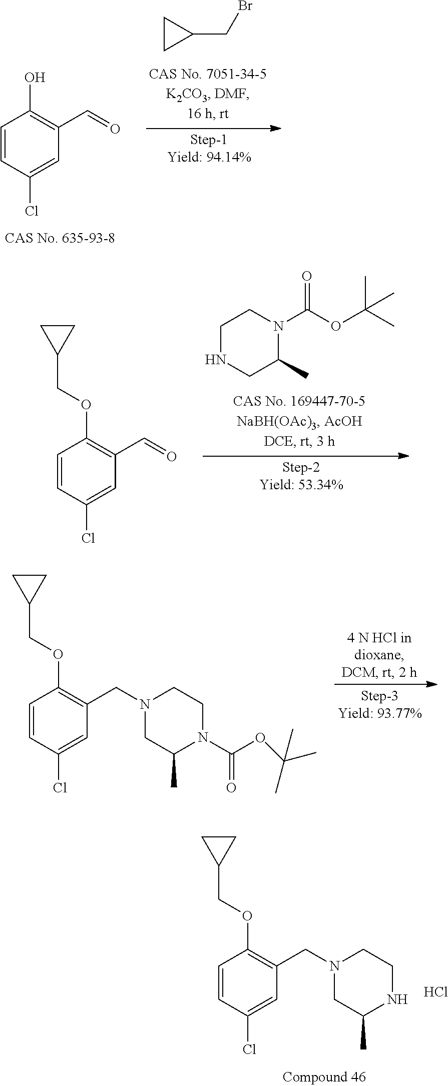

### Step-1: Synthesis of 5-chloro-2-(cyclopropylmethoxy) benzaldehyde

### Procedure

To a stirred solution of 5-chloro-2-hydroxybenzaldehyde (3 g, 19.16 mmol) in DMF (30 mL, 10V) at room temperature, K₂CO₃ (7.94 g, 57.48 mmol) was added. The reaction mixture was stirred for 1 h. Then (bromomethyl)cyclopropane (3.8 g, 28.6 mmol) was added to the reaction mixture. The reaction mixture was stirred at room temperature for 16 h. The progress of the reaction was monitored by TLC. After completion of the reaction, the reaction mixture was poured into ice-cold water (200 mL) and extracted with ethyl acetate (3×150 mL). The combined organic fractions were washed with cold water (2×60 mL), dried over sodium sulphate and concentrated under reduced pressure to provide 5-chloro-2-(cyclopropylmethoxy) benzaldehyde (3.82, 94.14% yield) as a light brown solid.

LCMS [ESI, M+1]: 210.8 (RT: 2.258 min, Purity: 100.00%).

### Step-2: Synthesis of tert-butyl (S)-4-(5-chloro-2-(cyclopropylmethoxy)benzyl)-2-methylpiperazine-1-carboxylate

### Procedure

To a stirred solution of 5-chloro-2-(cyclopropylmethoxy) benzaldehyde (0.2 g, 0.94 mmol) in DCE (2.0 mL, 10V) at room temperature, tert-butyl (S)-2-methylpiperazine-1-carboxylate (0.228 g, 1.13 mmol) and acetic acid (0.0025 mL, 0.047 mmol) were added. The reaction mixture was stirred at room temperature for 2 h. Sodium triacetoxyborohydride (0.603 g, 2.84 mmol) was added to the reaction mixture portion-wise at 0° C. The reaction was stirred at room temperature for 3 h. The progress of the reaction was monitored by TLC and LCMS analysis. After completion of the reaction, the reaction mixture was poured into a saturated solution of sodium bicarbonate (15 mL) and extracted with CH₂Cl₂ (3×15 mL). The combined organic fractions were washed with water (15 mL), dried over sodium sulphate and concentrated under reduced pressure. The crude material was purified by trituration using n-pentane (2×15 mL) to provide tert-butyl (S)-4-(5-chloro-2-(cyclopropylmethoxy)benzyl)-2-methylpiperazine-1-carboxylate (0.2 g, 53.34% yield) as a yellowish sticky solid.

LCMS [ESI, M&M+2]: 395.1, 396.8 (RT: 1.742 min, Purity: 100.00%).

### Step-3: Synthesis of (S)-1-(5-chloro-2-(cyclopropylmethoxy)benzyl)-3-methylpiperazine hydrochloride (Compound 46)

### Procedure

To a stirred solution of Tert-butyl (S)-4-(5-chloro-2-(cyclopropylmethoxy) benzyl)-2-methylpiperazine-1-carboxylate (0.2 g, 0.50 mmol) in CH₂Cl₂ (2.0 mL, 10V) at 0° C., 4M HCl in dioxane (1.0 mL, 5V) was added. The reaction mixture was stirred at room temperature for 2 h. The progress of the reaction was monitored by TLC and LCMS analysis. After completion of the reaction, the reaction mixture was concentrated under reduced pressure and the crude material was purified by trituration using diethyl ether (2×10 mL) and n-pentane (2×10 mL) to provide (S)-1-(5-chloro-2-(cyclopropylmethoxy)benzyl)-3-methylpiperazine hydrochloride (0.14 g, 93.77% yield) as a pale yellow solid.

LCMS [ESI, M&M+2]: 294.9, 296.6 (RT: 1.139 min, Purity: 98.48%),

HPLC: RT: 4.433, Purity: 99.19%

Chiral HPLC: RT: 2.71, Purity: 97.98%

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% METHANOLIC AMMONIA in METHANOL with 80-20 composition method; with Flow rate=3 ml/min; Column oven temperature 40° C.; ABPR 130 bar, analysis time 6 min.

¹H NMR (400 MHz, d₆-DMSO) δ: 12.03-11.78 (m, 1H), 9.68 (s, 2H), 7.66 (s, 1H), 7.45 (s, 1H), 7.11 (d, J=8.9 Hz, 1H), 4.25 (s, 2H), 3.91 (p, J=10.2 Hz, 2H), 3.75-3.60 (m, 1H), 3.30-3.00 (m, 4H), 1.27 (t, J=9.2 Hz, 4H), 0.67-0.55 (m, 2H), 0.36 (q, J=4.9 Hz, 2H).

### Experimental Protocol for Compound 47

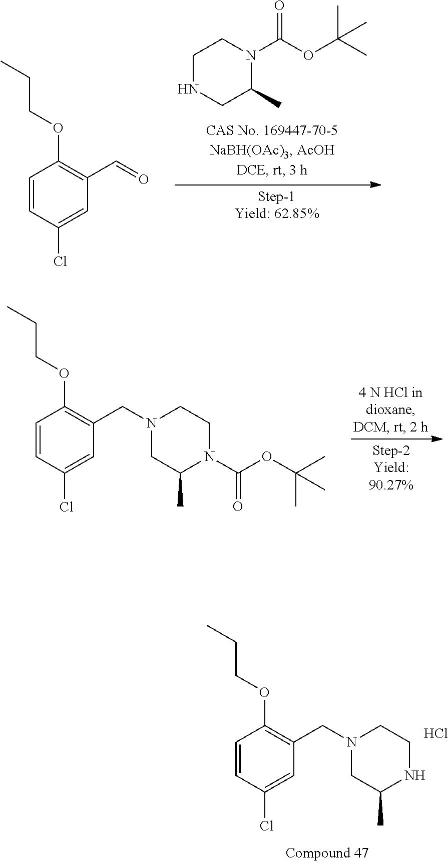

### Step-1: Synthesis of tert-butyl (S)-4-(5-chloro-2-propoxybenzyl)-2-methylpiperazine-1-carboxy late

### Procedure

To a stirred solution of 5-chloro-2-propoxybenzaldehyde (0.15 g, 0.75 mmol) in DCE (1.5 mL, 10V) at room temperature, tert-butyl (S)-2-methylpiperazine-1-carboxylate (0.18 g, 0.90 mmol) and acetic acid (0.015 mL, 0.037 mmol) were added. The reaction mixture was stirred at room temperature for 2 h. Sodium triacetoxyborohydride (0.48 g, 2.26 mmol) was added to the reaction mixture portion-wise at 0° C. The reaction was stirred at room temperature for 3 h. The progress of the reaction was monitored by TLC and LCMS analysis. After completion of the reaction, the reaction mixture was poured into a saturated solution of sodium bicarbonate (15 mL) and extracted with CH₂Cl₂ (3×15 mL). The combined organic fractions were washed with water (15 mL), dried over sodium sulphate and concentrated under reduced pressure. The crude material was purified by trituration using n-pentane (3×15 mL) to provide tert-butyl (S)-4-(5-chloro-2-propoxybenzyl)-2-methylpiperazine-1-carboxylate (0.18 g, 62.85% yield) as a yellow sticky solid.

LCMS [ESI (M,M+2)]: 383.0, 384.8 (RT: 1.708 min, Purity: 99.14%).

### Step-2: Synthesis of (S)-1-(5-chloro-2-propoxybenzyl)-3-methylpiperazine hydrochloride (Compound 47)

### Procedure

To a stirred solution of Tert-butyl (S)-4-(5-chloro-2-propoxybenzyl)-2-methylpiperazine-1-carboxylate (0.18 g, 0.46 mmol) in CH₂Cl₂ (1.8 mL, 10V) at 0° C., 4M HCl in dioxane (0.9 mL, 5V) was added. The reaction mixture was stirred at room temperature for 2 h. The progress of the reaction was monitored by TLC and LCMS analysis. After completion of the reaction, the reaction mixture was concentrated under reduced pressure and the crude material purified by trituration using diethyl ether (2×10 mL) and n-pentane (2×10 mL) to provide (S)-1-(5-chloro-2-propoxybenzyl)-3-methylpiperazine hydrochloride (0.12 g, 90.27%) as an off-white solid.

LCMS [ESI, M+1]: 283.0, 284.1 (RT: 1.124 min, Purity: 99.24%)

HPLC: RT: 4.419, Purity: 97.61%

Chiral HPLC: RT: 2.37, Purity: 97.07%

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% METHANOLIC AMMONIA in METHANOL with 80-20 composition method; with Flow rate=3 ml/min; Column oven temperature 40° C.; ABPR 130 bar, analysis time 6 min.

¹H NMR (400 MHz, d₆-DMSO) δ: 11.96 (s, 1H), 9.76 (s, 2H), 7.67 (s, 1H), 7.47 (d, J=7.4 Hz, 1H), 7.14 (d, J=8.9 Hz, 1H), 4.22 (s, 2H), 4.00 (t, J=6.4 Hz, 2H), 3.65 (dd, J=32.1, 27.2 Hz, 1H), 3.19-2.86 (m, 2H), 1.81-1.75 (m, 2H), 1.41-1.16 (m, 3H), 1.01 (t, J=7.4 Hz, 3H).

### Experimental Protocol for Compound 48

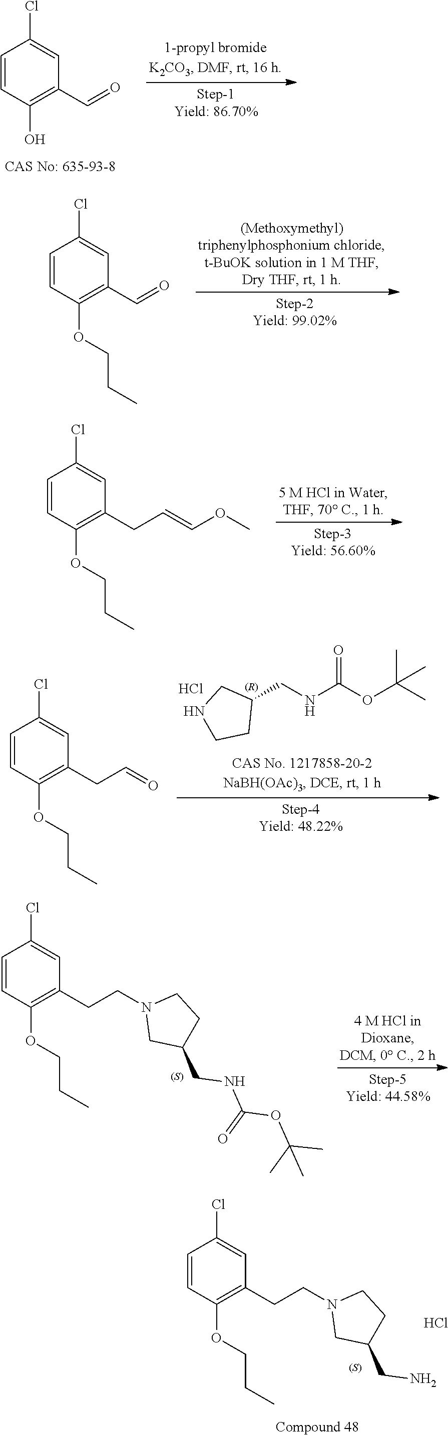

### Step-1: Synthesis of 5-chloro-2-propoxybenzaldehyde

### Procedure

To a stirred solution of 5-chloro-2-hydroxybenzaldehyde (5.0 g, 31.93 mmol) in DMF (50 mL) at room temperature, potassium carbonate (8.8 g, 63.86 mmol) was added. The reaction mixture was stirred at room temperature for 1 h. Then 1-propyl bromide (4.7 g, 38.92 mmol) was added to the reaction mixture. The reaction mixture was stirred at room temperature for 16 h. The progress of the reaction was monitored by TLC and LCMS analysis. After completion of the reaction, the reaction mixture was poured into ice cold water (80 mL). The solid precipitate that formed was isolated by filtration and dried under reduced pressure to provide 5-chloro-2-propoxybenzaldehyde (5.5 g, 86.50% yield) as a white solid.

LCMS [ESI, M+1]: 198.7 (RT: 2.175 min, Purity: 100%),

¹H NMR (400 MHz, d₆-DMSO): δ 10.32 (s, 1H), 7.70 (dd, J=8.9, 2.8 Hz, 1H), 7.62 (d, J=2.7 Hz, 1H), 7.28 (d, J=9.0 Hz, 1H), 4.10 (t, J=7.0 Hz, 2H), 1.80 (q, J=6.9 Hz, 2H), 1.01 (t, J=7.0 Hz, 3H).

### Step-2: Synthesis of (E)-4-chloro-2-(3-methoxyallyl)-1-propoxybenzene

### Procedure

To a stirred solution of (Methoxymethyl)triphenyl phosphine (2.1 g, 6.29 mmol) in anhydrous THF (20 mL) at room temperature, t-BuOK in 1M THF (12.5 mL, 12.58 mmol) was added. The reaction mixture was stirred at room temperature for 1 h. Then 5-chloro-2-propoxybenzaldehyde (0.5 g, 2.51 mmol) dissolved in anhydrous THF was added drop-wise to the reaction mixture at room temperature. The reaction was stirred at room temperature for 1 h. The progress of the reaction was monitored by TLC. After completion of the reaction, the reaction mixture was concentrated under reduced pressure and the crude material purified by column chromatography (Alumna oxide neutral; 0-2% ethyl acetate in hexane) to provide (E)-4-chloro-2-(3-methoxyallyl)-1-propoxybenzene (0.6 g, 99.02% yield) as a light yellow liquid.

¹H NMR (400 MHz, d₆-DMSO): δ 7.27-7.21 (m, 1H), 7.14-7.06 (m, 1H), 6.94 (dd, J=8.8, 3.4 Hz, 1H), 6.41 (d, J=7.2 Hz, 1H), 5.50 (d, J=7.2 Hz, 1H), 3.94 (q, J=11.0 Hz, 2H), 3.75 (s, 3H), 1.79-1.70 (m, 4H), 0.98 (dt, J=7.4, 5.8 Hz, 3H).

### Step-3: Synthesis of 2-(5-chloro-2-propoxyphenyl)acetaldehyde

### Procedure

To a stirred solution of (E)-4-chloro-2-(3-methoxyallyl)-1-propoxybenzene (0.6 g, 2.64 mmol) in THF (6 mL) at room temperature, 5M HCl in water (3 mL, 5V) was added. The reaction mixture was stirred at 70° C. for 1 h. The progress of the reaction was monitored by TLC. After completion of the reaction, the reaction mixture was poured into a saturated solution of sodium bicarbonate (80 mL) and extracted with ethyl acetate (3×30 mL). The combined organic fractions were dried over sodium sulphate and concentrated under reduced pressure to provide 2-(5-chloro-2-propoxyphenyl)acetaldehyde (0.3 g, 56.60% yield) as a light yellow liquid.

¹H NMR (400 MHz, d₆-DMSO): δ 9.63 (t, J=1.5 Hz, 1H), 7.34-7.28 (m, 2H), 7.08-6.99 (m, 1H), 3.91 (q, J=6.6 Hz, 2H), 3.73-3.64 (m, 2H), 1.68 (dd, J=13.7, 6.4 Hz, 2H), 0.95 (t, J=13.3 Hz, 3H).

### Step-4: Synthesis of tert-butyl (S)-((1-(5-chloro-2-propoxyphenethyl)pyrrolidin-3-yl)methyl) carbamate

### Procedure

To a stirred solution of 2-(5-chloro-2-propoxyphenyl)acetaldehyde (0.3 g, 1.41 mmol) in DCE (6 mL) at room temperature, tert-butyl (R)-(pyrrolidin-3-ylmethyl)carbamate hydrochloride (0.401 g, 1.69 mmol) was added. The reaction mixture was stirred at room temperature for 1 h. Sodium triacetoxyborohydride (0.895 g, 4.24 mmol) was added portion-wise to the reaction mixture at 0° C. The reaction was stirred at room temperature for 1 h. The progress of the reaction was monitored by TLC and LCMS analysis. After completion of the reaction, the reaction mixture was poured into a saturated solution of sodium bicarbonate (30 mL) and extracted with CH₂Cl₂ (3×20 mL). The combined organic fractions were washed with water (25 mL), dried over sodium sulphate and concentrated under reduced pressure. The crude material was purified by column chromatography (Aluminum oxide Neutral; 30-35% ethyl acetate in hexane) to provide tert-butyl (S)-((1-(5-chloro-2-propoxyphenethyl)pyrrolidin-3-yl)methyl)carbamate (0.18 g, 48.22% yield) as a light yellow liquid.

LCMS [ESI, M+1]: 396.9 (RT: 1.702 min, Purity: 86.32%),

¹H NMR (400 MHz, d₆-DMSO): δ 7.25-7.20 (m, 1H), 7.19 (dd, J=8.7, 2.7 Hz, 1H), 6.95 (dd, J=8.6, 5.0 Hz, 1H), 6.88 (t, J=5.5 Hz, 1H), 3.97-3.87 (t, J=11.2 Hz, 2H), 2.88 (t, J=6.4 Hz, 2H), 2.70 (t, J=7.2 Hz, 2H), 2.5 (m, 4H), 2.30-2.08 (m, 3H), 1.83-1.67 (m, 4H), 1.43-1.33 (m, 9H), 1.05-0.96 (t, J=13.3 Hz, 3H).

### Step-5: Synthesis of (S)-(1-(5-chloro-2-propoxyphenethyl)pyrrolidin-3-yl)methanamine hydrochloride (Compound 48)

### Procedure

To a stirred solution of tert-butyl (S)-((1-(5-chloro-2-propoxyphenethyl)pyrrolidin-3-yl)methyl) carbamate (0.18 g, 0.45 mmol) in CH₂Cl₂ (1.8 mL) at 0° C., 4M HCl in dioxane (0.09 mL, 2V) was added. The reaction mixture was stirred at 0° C. for 2 h. The progress of the reaction was monitored by TLC and LCMS analysis. After completion of the reaction, the reaction mixture was concentrated under reduced pressure and the crude material purified by preparative HPLC (0.05% HCl in water/acetonitrile) to provide (S)-(1-(5-chloro-2-propoxyphenethyl)pyrrolidin-3-yl)methanamine hydrochloride (0.06 g, 44.58% yield) as a yellow liquid.

LCMS [ESI, M+1]: 296.8 (RT: 0.843 min, Purity: 100%),

HPLC: RT: 4.453 min, Purity: 100%,

Chiral HPLC: RT: 1.97 min, Purity: 100%,

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% DIETHYLAMINE in METHANOL with 70-30 composition method; with Flow rate=3 ml/min; Column oven temperature 40° C.; ABPR 130 bar, analysis time 6 min.

¹H NMR (400 MHz, d₆-DMSO): δ 11.23 (s, 1H), 8.18 (s, 3H), 7.30 (dt, J=20.5, 10.3 Hz, 2H), 7.04 (d, J=8.5 Hz, 1H), 3.98 (t, J=6.4 Hz, 2H), 3.69 (d, J=46.8 Hz, 2H), 3.37-3.23 (m, 3H), 3.10-2.87 (m, 5H), 2.26 (m, 1H), 2.10 (m, 1H), 1.93 (m, 1H), 1.75 (q, J=11.9 Hz, 2H), 1.03 (t, J=7.4 Hz, 3H).

### Experimental Protocol for Compound 49

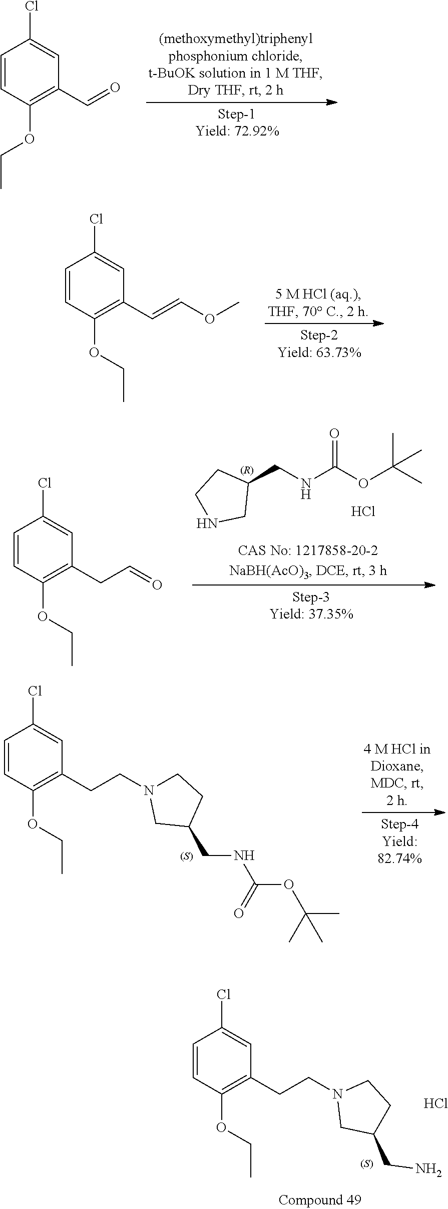

### Step-1: Synthesis of (E)-4-chloro-1-ethoxy-2-(2-methoxyvinyl)benzene

### Procedure

To a stirred solution of (methoxymethyl)triphenyl phosphonium chloride, (2.3 g, 6.77 mmol) in anhydrous THF (10 mL, 20V) at 0° C., t-BuOK solution in 1M THF (13.5 mL) was added. The reaction mixture was stirred at room temperature for 1.5 h. 5-chloro-2-ethoxybenzaldehyde (0.5 g, 2.70 mmol) in THF (3 mL) was added drop-wise to the reaction mixture at 0° C. The reaction was stirred at room temperature for 30 min. The progress of the reaction was monitored by TLC. After completion of the reaction, the reaction mixture was concentrated under reduced pressure and the crude material purified by column chromatography (neutral silica; 100% hexane) to provide (E)-4-chloro-1-ethoxy-2-(2-methoxyvinyl)benzene (0.42 g, 72.92% yield) as a light yellow liquid which was directly used for next step without analysis.

### Step-2: Synthesis of 2-(5-chloro-2-ethoxyphenyl) acetaldehyde

### Procedure

To a stirred solution of (E)-4-chloro-1-ethoxy-2-(2-methoxyvinyl)benzene (0.42 g, 1.974 mmol) in anhydrous THF (4.2 mL, 10V) at 0° C., 5M HCl solution (2.1 mL, 5V) was added. The reaction mixture was stirred at 70° C. for 2 h. The progress of the reaction was monitored by TLC. After completion of the reaction, the reaction mixture was poured into a saturated solution of sodium bicarbonate (30 mL) and extracted with Ethyl Acetate (2×15 mL). The combined organic fractions were washed with water (20 mL), dried over sodium sulphate and concentrated under reduced pressure to provide 2-(5-chloro-2-ethoxyphenyl)acetaldehyde (0.25 g, 63.73% yield) as an off-white sticky solid.

¹H NMR (400 MHz, d₆-DMSO) δ 9.62 (s, 1H), 7.00-6.83 (m, 1H), 6.65-6.56 (m, 1H), 6.37-6.18 (m, 1H), 4.12 (q, J=6.9 Hz, 2H), 3.70 (m, 2H), 0.62 (t, J=7.1 Hz, 3H).

### Step-3: Synthesis of tert-butyl (S)-((1-(5-chloro-2-ethoxyphenethyl)pyrrolidin-3-yl)methyl)carbamate

### Procedure

To a stirred solution of 2-(5-chloro-2-ethoxyphenyl)acetaldehyde (0.25 g, 1.26 mmol) in DCE (2.5 mL) at room temperature, tert-butyl (R)-(pyrrolidin-3-ylmethyl)carbamate hydrochloride (0.358 g, 1.51 mmol) was added. The reaction mixture was stirred at room temperature for 1 h. Sodium triacetoxyborohydride (0.798 g, 3.78 mmol) was added portion-wise to the reaction mixture at 0° C. The reaction was stirred at room temperature for 3 h. The progress of the reaction was monitored by TLC and LCMS analysis. After completion of reaction, the reaction mixture was poured into a saturated solution of sodium bicarbonate (30 mL) and extracted with CH₂Cl₂ (3×25 mL). The combined organic fractions were washed with water (2×20 mL), dried over sodium sulphate and concentrated under reduced pressure. The crude material was purified by flash column chromatography (neutral silica; 20% ethyl acetate in hexane) to provide tert-butyl (S)-((1-(5-chloro-2-ethoxyphenethyl)pyrrolidin-3-yl)methyl)carbamate (0.18 g, 37.35% yield) as a light yellow liquid.

LCMS [ESI, M+2]: 382.8 (RT: 1.560 min, Purity: 90.31%).

### Step-4: Synthesis of (S)-(1-(5-chloro-2-ethoxyphenethyl)pyrrolidin-3-yl)methanamine hydrochloride (Compound 49)

### Procedure

To a stirred solution of tert-butyl(S)-((1-(5-chloro-2-ethoxyphenethyl)pyrrolidin-3-yl)methyl)carbamate (0.18 g, 0.47 mmol) in CH₂Cl₂ (1.8 mL, 10V) at 0° C., 4M HCl in dioxane (0.5 mL, 3.0V) was added. The reaction mixture was stirred at room temperature for 2 h. The progress of the reaction was monitored by TLC. After completion of the reaction, the reaction mixture was concentrated under reduced pressure and the crude material triturated with diethyl ether (3×10 mL) to provide (S)-(1-(5-chloro-2-ethoxyphenethyl)pyrrolidin-3-yl)methanamine hydrochloride (0.11 g, 82.74% yield) as an off-white solid.

LCMS [ESI, M+1]: 282.7 (RT: 0.975 min, Purity: 98.53%),

HPLC: RT: 4.213 min, Purity: 95.10%,

Chiral HPLC: RT: 3.27 min, Purity: 97.45%,

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: 0.1% DIETHYLAMINE in METHANOL with 80-20 composition method; with Flow rate=3 ml/min; Column oven temperature 40° C.; ABPR 130 bar, analysis time 10 min.

¹H NMR (400 MHz, CD₃OD) δ 7.31 (d, J=2.5 Hz, 1H), 7.26 (dd, J=8.7, 2.5 Hz, 1H), 6.99 (d, J=8.7 Hz, 1H), 4.12 (q, J=6.9 Hz, 2H), 3.94 (dd, J=19.4, 11.3 Hz, 1H), 3.85 (dd, J=19.9, 10.5 Hz, 1H), 3.60 (d, J=4.1 Hz, 1H), 3.45 (d, J=6.1 Hz, 2H), 3.29-3.14 (m, 2H), 3.16-3.08 (m, 2H), 3.00 (d, J=11.1 Hz, 1H), 2.78 (s, 1H), 2.56-2.24 (m, 1H), 1.96 (d, J=61.1 Hz, 1H), 1.48 (t, J=7.0 Hz, 3H).

### Experimental Protocol for Compound 50

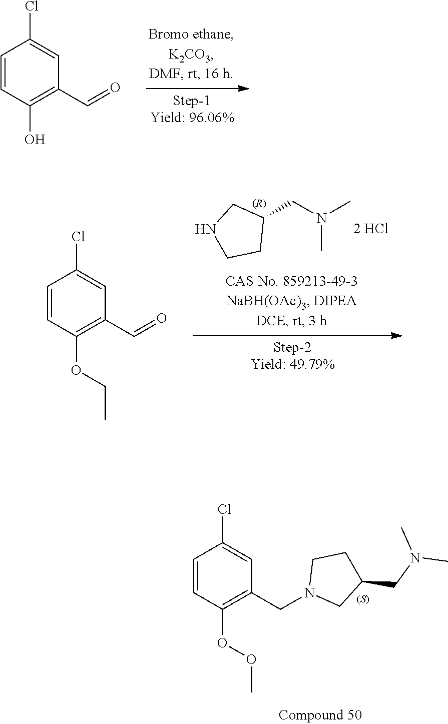

### Step-1: Synthesis of 5-chloro-2-ethoxybenzaldehyde

### Procedure

To a stirred solution of 5-chloro-2-hydroxybenzaldehyde (15.0 g, 95.84 mmol) in DMF (150 mL) at room temperature, potassium carbonate (33.09 g, 239.6 mmol) was added. The reaction mixture was stirred at room temperature for 1 h. Bromoethane (10.71 mL, 124.60 mmol) was added and the reaction mixture stirred at room temperature for 16 h. The progress of the reaction was monitored by TLC. After completion of the reaction, the reaction mixture was poured into cold water (250 mL). The solid precipitate was isolated by filtration, washed with n-hexane and dried under reduced pressure to provide 5-chloro-2-ethoxybenzaldehyde (17.0 g, 96.06% yield) as a white solid.

¹H NMR (400 MHz, CDCl₃): δ 10.45 (s, 1H), 7.80 (dd, J=8.9, 2.8 Hz, 1H), 7.50 (d, J=2.7 Hz, 1H), 6.96 (d, J=9.0 Hz, 1H), 4.19 (q, J=6.9 Hz, 2H), 1.50 (t, J=7.0 Hz, 3H).

### Step-2: Synthesis of (S)-1-(1-(5-chloro-2-ethoxybenzyl)pyrrolidin-3-yl)-N,N-dimethyl methanamine (Compound 50)

### Procedure

To a stirred solution of 5-chloro-2-ethoxybenzaldehyde (0.15 g, 0.81 mmol) in DCE (2 mL) at room temperature, (R)—N,N-dimethyl-1-(pyrrolidin-3-yl)methanamine dihydrochloride (0.196 g, 0.97 mmol) and DIPEA (0.262 g, 2.02 mmol) were added. The reaction mixture was stirred at room temperature for 1 h. Sodium triacetoxyborohydride (0.429 g, 2.02 mmol) was added portion-wise to the reaction mixture at 0° C. The reaction mixture was allowed to stir at room temperature for 3 h. The progress of the reaction was monitored by TLC. After completion of the reaction, the reaction mixture was poured into water (30 mL) and extracted with CH₂Cl₂ (3×50 mL). The combined organic fractions were washed with sat. NaHCO₃ solution (2×20 mL), dried over anhydrous sodium sulphate and concentrated under reduced pressure. The crude material was purified by reverse phase column (C18 silica; 0-10% [0.05% HCl in H₂O/CH₃CN]) to provide (S)-1-(1-(5-chloro-2-ethoxybenzyl)pyrrolidin-3-yl)-N,N-dimethylmethanamine (0.12 g, 49.79% yield) as a yellow sticky solid.

LCMS [ESI, M+1]: 296.6 (RT: 0.853 min, Purity: 100%),

HPLC Purity: RT: 3.927 min, Purity: 100%,

Chiral HPLC: RT: 1.93 min, Purity: 100%,

Instrument Name: Waters SFC Investigator

Chromatographic separation was conducted with Waters SFC Investigator system with 2998 PDA detector. The column used was CHIRALPAK® IA (250×4.6 mm; 5 ␣m) and the compounds were eluted with, Mobile Phase A: Liq·CO2, Mobile Phase B: 0.1% DIETHYLAMINE in METHANOL with 60-40 composition method; with Flow rate=3 ml/min; Column oven temperature 40° C.; ABPR 130 bar, analysis time 5 min.

¹H NMR (400 MHz, CD₃OD) δ 7.59 (d, J=6.4 Hz, 1H), 7.48 (d, J=8.6 Hz, 1H), 7.14 (d, J=8.8 Hz, 1H), 4.47 (q, J=12.4 Hz, 2H), 4.22 (q, J=6.6 Hz, 2H), 3.83 (s, 1H), 3.66 (s, 2H), 3.42 (d, J=27.5 Hz, 3H), 3.14 (s, 1H), 2.96 (s, 6H), 2.50 (m, 1H), 2.12-1.97 (m, 1H), 1.51 (t, J=6.9 Hz, 3H).

### Experimental Protocol for Compound 51

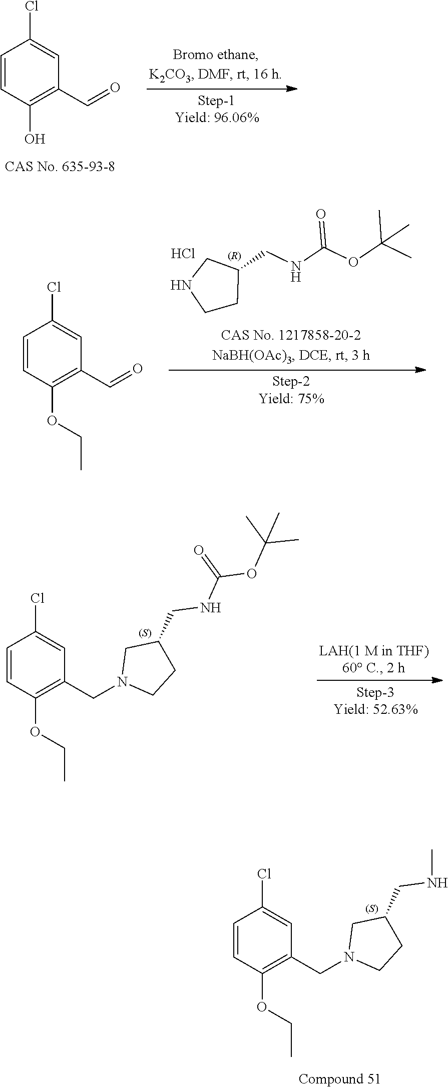

### Step-1: Synthesis of 5-chloro-2-ethoxybenzaldehyde

### Procedure

To a stirred solution of 5-chloro-2-hydroxybenzaldehyde (15.0 g, 95.84 mmol) in DMF (150 mL) at room temperature, potassium carbonate (33.09 g, 239.6 mmol) was added. The reaction mixture was stirred at room temperature for 1 h. Bromoethane (10.71 mL, 124.60 mmol) was added into reaction mixture. The reaction mixture was stirred at room temperature for 16 h. The progress of the reaction was monitored by TLC. After completion of the reaction, the reaction mixture was poured into cold water (300 mL). The solid precipitate was isolated by filtration, washed with hexane (2×30 mL) and dried under reduced pressure to provide 5-chloro-2-ethoxybenzaldehyde (17.0 g, 96.06% yield) as a white solid.

¹H NMR (400 MHz, CDCl₃): δ 10.45 (s, 1H), 7.80 (dd, J=8.9, 2.8 Hz, 1H), 7.50 (d, J=2.7 Hz, 1H), 6.96 (d, J=9.0 Hz, 1H), 4.19 (q, J=6.9 Hz, 2H), 1.50 (t, J=7.0 Hz, 3H).

### Step-2: Synthesis of tert-butyl (S)-((1-(5-chloro-2-ethoxybenzyl)pyrrolidin-3-yl)methyl) carbamate

### Procedure

To a stirred solution of 5-chloro-2-ethoxybenzaldehyde (0.8 g, 4.34 mmol) in DCE (8 mL) at room temperature, tert-butyl (R)-(pyrrolidin-3-ylmethyl)carbamate hydrochloride (1.4 g, 5.21 mmol) was added. The reaction mixture was stirred at room temperature for 1 h. Sodium triacetoxyborohydride (2.29 g, 10.85 mmol) was added portion-wise to the reaction mixture at 0° C. The reaction mixture was allowed to stir at room temperature for 3 h. The progress of the reaction was monitored by TLC. After completion of the reaction, the reaction mixture was poured into water (80 mL) and extracted in CH₂Cl₂ (3×30 mL). The combined organic fractions were washed with sat. NaHCO₃ solution (30 mL), dried over anhydrous sodium sulphate and concentrated under reduced pressure. The crude material was purified by column chromatography (neutral alumina; 10-15% EtOAc/hexane) to provide tert-butyl (S)-((1-(5-chloro-2-ethoxybenzyl) pyrrolidin-3-yl)methyl) carbamate (1.2 g, 75% yield) as a yellow oil.

LCMS [ESI, M+1]: 369.0 (RT: 1.461 min, Purity: 99.07%).

### Step-3: Synthesis of (S)-1-(1-(5-chloro-2-ethoxybenzyl)pyrrolidin-3-yl)-N-methylmethanamine (Compound 51)

### Procedure

To a stirred solution of tert-butyl (S)-((1-(5-chloro-2-ethoxybenzyl) pyrrolidin-3-yl)methyl) carbamate (0.3 g, 0.81 mmol) in anhydrous THF (5.0 mL) at 0° C., LiAlH₄ (1M in THF) (1.2 mL, 2.44 mmol) was added. The reaction mixture was stirred at 60° C. for 2 h. The progress of the reaction was monitored by TLC and LCMS analysis. After completion of the reaction, the reaction mixture was quenched with a saturated solution of NH₄Cl (20 mL) and extracted with ethyl acetate (3×20 mL). The combined organic fractions were washed with water (2×15 mL), dried over sodium sulphate and concentrated under reduced pressure. The crude material was purified by reverse phase column chromatography (0-10% [0.05% HCl in water and MeCN]) to provide (S)-1-(1-(5-chloro-2-ethoxybenzyl)pyrrolidin-3-yl)-N-methylmethanamine (0.12 g, 52.63% yield) as a light yellow solid.

LCMS [ESI, M+1]: 282.7 (RT: 0.844 min, Purity: 98.98%),

HPLC Purity: RT: 4.020 min, Purity: 95.68%,

Chiral HPLC: RT: 1.90 min, Purity: 100%,

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: METHANOL with 75-25 composition method; with Flow rate=3 ml/min; Column oven temperature 40° C.; ABPR 130 bar, analysis time 6 min.

¹H NMR (400 MHz, CD₃OD) δ 7.59 (dd, J=8.4, 2.5 Hz, 1H), 7.47 (d, J=8.1 Hz, 1H), 7.13 (d, J=8.9 Hz, 1H), 4.51-4.40 (m, 2H), 4.22 (dt, J=13.0, 6.5 Hz, 2H), 3.73 (ddd, J=28.8, 14.4, 8.8 Hz, 2H), 3.50-3.40 (m, 1H), 3.26-3.11 (m, 3H), 3.06-2.84 (m, 1H), 2.74 (t, J=8.3 Hz, 3H), 2.41 (ddd, J=20.7, 12.8, 7.0 Hz, 1H), 1.99 (ddd, J=40.3, 21.6, 12.8 Hz, 1H), 1.50 (t, J=7.0 Hz, 3H).

### Experimental Protocol for Compound 52

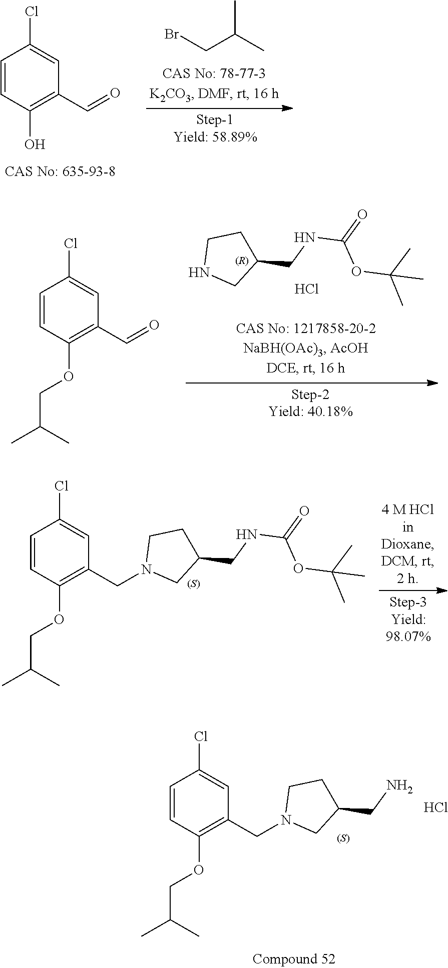

### Step-1: Synthesis of 5-chloro-2-isobutoxybenzaldehyde

### Procedure

To a stirred solution of 5-chloro-2-hydroxybenzaldehyde (0.25 g, 1.308 mmol) in DMF (2.5 mL) at room temperature, potassium carbonate (0.541 g, 3.924 mmol) was added. The reaction mixture was stirred for 1 h. 1-bromo-2-methylpropane (0.269 g, 1.963 mmol) was added. The reaction mixture was stirred at room temperature for 16 h. The progress of the reaction was monitored by TLC. After completion of the reaction, the reaction mixture was poured into ice-cold water (30 mL) and extracted with ethyl acetate (3×20 mL). The combined organic fractions were dried over sodium sulphate and concentrated under reduced pressure. The crude material was purified by column chromatography (SiO₂; 21% ethyl acetate in hexane) to provide 5-chloro-2-isobutoxybenzaldehyde (0.2 g, 58.89% yield) as an off white sticky liquid.

¹H NMR (400 MHz, d₆-DMSO) δ 10.33 (s, 1H), 7.69-7.52 (m, 2H), 7.27 (d, J=9.0 Hz, 1H), 3.91 (d, J=6.4 Hz, 2H), 2.23-1.87 (m, 1H), 1.01 (d, J=6.7 Hz, 6H).

### Step-2: Synthesis of tert-butyl (S)-((1-(5-chloro-2-isobutoxybenzyl)pyrrolidin-3-yl)methyl)carbamate

### Procedure

To a stirred solution of 5-chloro-2-isobutoxybenzaldehyde (0.2 g, 0.943 mmol) in DCE (3 mL) at room temperature, tert-butyl (R)-(pyrrolidin-3-ylmethyl) carbamate hydrochloride (0.267 g, 1.132 mmol) and acetic acid (0.01 mL, 0.05V) were added. The reaction mixture was stirred at room temperature for 1 h. Sodium triacetoxyborohydride (0.596 g, 2.829 mmol) was added to the reaction mixture portion-wise at 0° C. The reaction was stirred at room temperature for 16 h. The progress of the reaction was monitored by TLC and LCMS analysis. After completion of the reaction, the reaction mixture was poured into a saturated solution of sodium bicarbonate (30 mL) and extracted with CH₂Cl₂ (3×20 ml). The combined organic fractions were washed with water (30 mL), dried over sodium sulphate and concentrated under reduced pressure. The crude material was purified by column chromatography (SiO₂; 60-65% ethyl acetate in hexane) to provide tert-butyl (S)-((1-(5-chloro-2-isobutoxybenzyl)pyrrolidin-3-yl)methyl)carbamate (0.15 g, 40.18% yield) as a colourless liquid.

LCMS [ESI, M+1]: 396.8 (RT: 1.698 min, Purity: 100%)

### Step-3: Synthesis of (S)-(1-(5-chloro-2-isobutoxybenzyl)pyrrolidin-3-yl)methanamine hydrochloride (Compound 52)

### Procedure

To a stirred solution of tert-butyl (S)-((1-(5-chloro-2-isobutoxybenzyl) pyrrolidin-3-yl)methyl)carbamate (0.15 g, 0.3 mmol) in CH₂Cl₂ (1.5 mL, 10V) at 0° C., 4M HCl in dioxane (0.7 mL, 5V) was added. The reaction mixture was stirred at room temperature for 2 h. The progress of the reaction was monitored by TLC and LCMS analysis. After completion of the reaction, the reaction mixture was concentrated under reduced pressure. The crude material was triturated with diethyl ether (2×15 mL) to provide (S)-(1-(5-chloro-2-isobutoxybenzyl) yrrolidine-3-yl)methanamine hydrochloride (0.11 g, 98.07%) as an off-white solid.

LCMS [ESI, M+1]: 296.8 (RT: 1.023 min, Purity: 100%),

HPLC: RT: 4.340 min, Purity: 95.50%

Chiral HPLC: RT: 2.04 min, Purity: 100%

Instrument Name: Waters SFC Investigator

Chromatographic separation was conducted with Waters SFC Investigator system with 2998 PDA detector. The column used was CHIRALCEL® OD-H (250×4.6 mm; 5 ␣m) and the compounds were eluted with, Mobile Phase A: Liq·CO2, Mobile Phase B: METHANOL with 75-25 composition method; with Flow rate=3 ml/min; Column oven temperature 40° C.; ABPR 130 bar, analysis time 6 min.

¹H NMR (400 MHz, D₂O) δ 7.41 (dd, J=8.8, 2.6 Hz, 1H), 7.38 (d, J=2.6 Hz, 1H), 7.03 (d, J=8.9 Hz, 1H), 4.32 (s, 2H), 3.83 (d, J=6.6 Hz, 2H), 3.60 (s, 1H), 3.41 (s, 2H), 3.06 (qd, J=13.1, 7.5 Hz, 3H), 2.73 (s, 1H), 2.29 (s, 1H), 2.04 (dq, J=13.5, 6.7 Hz, 1H), 1.81 (s, 1H), 0.95 (d, J=6.7 Hz, 6H).

### Experimental Protocol for Compound 53

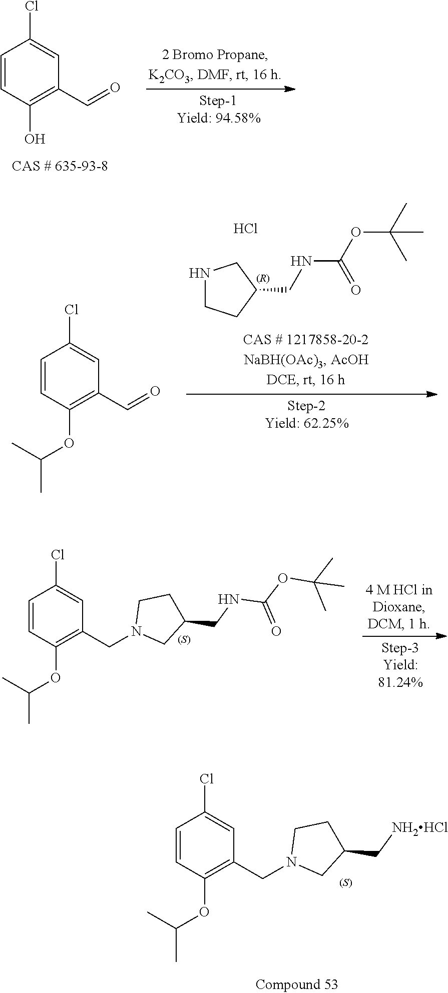

### Step-1: Synthesis of 5-chloro-2-isopropoxybenzaldehyde

### Procedure

To a stirred solution of 5-chloro-2-hydroxybenzaldehyde (0.1 g, 1.27 mmol) in DMF (1 mL, 10V) at room temperature, potassium carbonate (0.176 g, 1.27 mmol) was added. The reaction mixture was stirred at room temperature for 1 h. 2-Bromo propane (0.155 g, 1.27 mmol) was added to the reaction mixture. The reaction mixture was stirred at room temperature for 16 h. The progress of the reaction was monitored by TLC and LCMS analysis. After completion of the reaction, the reaction mixture was poured into cold water (20 mL) and extracted with ethyl acetate (3×10 mL). The combined organic fractions were washed with cold water (4×10 mL), dried over sodium sulphate and concentrated under reduced pressure to provide 5-chloro-2-isopropoxybenzaldehyde (0.120 g, 94.58% yield) as an off-white solid.

¹H NMR (400 MHz, d₆-DMSO): δ 10.29 (s, 1H), 7.65 (dt, J=16.9, 8.4 Hz, 1H), 7.58 (t, J=9.2 Hz, 1H), 7.32 (d, J=9.0 Hz, 1H), 4.88-4.72 (m, 1H), 1.33 (d, J=6.0 Hz, 6H).

### Step-2: Synthesis of tert-butyl (S)-((1-(5-chloro-2-isopropoxybenzyl)pyrrolidin-3-yl)methyl)carbamate

### Procedure

To a stirred solution of 5-chloro-2-isopropoxybenzaldehyde (0.1 g, 0.50 mmol) in DCE (2 mL, 20V) at room temperature, tert-butyl (R)-(pyrrolidin-3-ylmethyl)carbamate hydrochloride (0.12 g, 0.60 mmol) and acetic acid (0.002 mL, 0.05V) were added. The reaction mixture was stirred at room temperature for 2 h. Sodium triacetoxyborohydride (0.321 g, 1.5 mmol) was added portion-wise to the reaction mixture at 0° C. The reaction mixture was stirred at room temperature for 16 h. The progress of the reaction was monitored by TLC and LCMS analysis. After completion of the reaction, the reaction mixture was poured into a saturated solution of sodium bicarbonate (15 mL) and extracted with CH₂Cl₂ (3×10 mL). The combined organic fractions were washed with water (20 mL), dried over sodium sulphate and concentrated under reduced pressure. The crude material was purified by column chromatography (SiO₂; 75% ethyl acetate in hexane) to provide tert-butyl (S)-((1-(5-chloro-2-isopropoxybenzyl)pyrrolidin-3-yl)methyl)carbamate (0.12 g, 62.25% yield) as a light yellow liquid.

LCMS [ESI, M+1]: 382.8 (RT: 1.661 min, Purity: 100%),

CHIRAL HPLC: (RT: 2.51 min, Purity: 98.67%),

¹H NMR (400 MHz, d₆-DMSO): δ 7.29 (d, J=2.7 Hz, 1H), 7.20 (dd, J=8.8, 2.7 Hz, 1H), 6.98 (d, J=8.8 Hz, 1H), 6.86 (t, J=9.8 Hz, 1H), 4.60-4.54 (m, 1H), 3.50 (s, 2H), 2.89 (t, J=9.8 Hz, 4H), 2.20 (m, 2H), 1.90 (s, 2H), 1.80 (m, 1H), 1.36 (s, 9H), 1.25 (d, J=10.5 Hz, 6H).

### Step-3: Synthesis of (S)-(1-(5-chloro-2-isopropoxybenzyl)pyrrolidin-3-yl)methanamine hydrochloride (Compound 53)

### Procedure

To a stirred solution of tert-butyl (S)-((1-(5-chloro-2-isopropoxybenzyl)pyrrolidin-3-yl)methyl)carbamate (0.12 g, 0.31 mmol) in CH₂Cl₂ (1.2 mL, 10V) at 0° C., 4M HCl in dioxane (0.6 mL, 5V) was added. The reaction mixture was stirred at room temperature for 1 h. The progress of the reaction was monitored by TLC and LCMS analysis. After completion of the reaction, the reaction mixture was concentrated under reduced pressure and the crude material purified by trituration with diethyl ether (2×10 mL) to provide (S)-(1-(5-chloro-2-isopropoxybenzyl)pyrrolidin-3-yl)methanamine hydrochloride (0.072 g, 81.24% yield) as an off-white solid.

LCMS [ESI, M+1]: 282.8 (RT: 0.885 min, Purity: 98.75%),

HPLC: RT: 4.093 min, Purity: 100%,

CHIRAL HPLC: RT: 2.19 min, Purity: 100%

Instrument Name: Waters SFC Investigator

Chromatographic separation was conducted with Waters SFC Investigator system with 2998 PDA detector. The column used was CHIRALPAK IG (250×4.6 mm 5 um) and the compounds were eluted with, Mobile Phase A: Liq·CO2, Mobile Phase B: 0.1% DIETHYLAMINE in 2-PROPANOL-ACETONITRILE (70-30) with 50-50 composition method; with Flow rate=3 ml/min; Column oven temperature 40° C.; ABPR 130 bar, analysis time 7 min.

¹H NMR (400 MHz, CD₃OD): δ 7.60 (s, 1H), 7.46 (dd, J=8.9, 2.7 Hz, 1H), 7.16 (d, J=9.0 Hz, 1H), 4.76 (dt, J=12.0, 6.0 Hz, 1H), 4.43 (s, 2H), 3.75 (dd, J=17.3, 11.9 Hz, 1H), 3.64 (dd, J=26.4, 5.6 Hz, 1H), 3.48 (d, J=18.5 Hz, 1H), 3.28-3.05 (m, 3H), 2.87 (t, J=27.9 Hz, 1H), 2.36 (s, 1H), 1.95 (d, J=46.8 Hz, 1H), 1.43 (d, J=6.0 Hz, 6H).

### Experimental Protocol for Compound 54

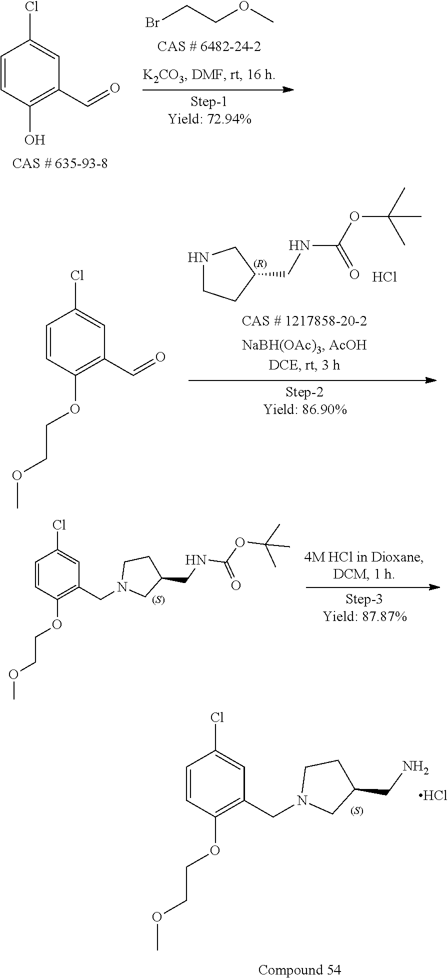

### Step-1: Synthesis of 5-chloro-2-(2-methoxyethoxy)benzaldehyde

### Procedure

To a stirred solution of 5-chloro-2-hydroxybenzaldehyde (0.5 g, 0.31 mmol) in DMF (5.0 mL) at room temperature, potassium carbonate (0.88 g, 0.63 mmol) was added. The reaction mixture was stirred at room temperature for 1 h. 1-bromo-2-methoxyethane (0.444 g, 0.31 mmol) was added into the reaction mixture. The reaction mixture was stirred at room temperature for 16 h. The progress of the reaction was monitored by TLC and LCMS analysis. After completion of the reaction, the reaction mixture was poured into cold water (50 mL) and extracted with ethyl acetate (3×25 mL). The combined organic fractions were washed with cold water (3×20 mL), dried over sodium sulphate and concentrated under reduced pressure to provide 5-chloro-2-(2-methoxyethoxy)benzaldehyde (0.55 g, 72.94% yield) as a colorless liquid.

LCMS [ESI, M+1]: 214.7 (RT: 1.716 min, Purity: 99.65%),

¹H NMR (400 MHz, d₆-DMSO): δ 10.32 (s, 1H), 7.73-7.64 (m, 1H), 7.61 (t, J=10.3 Hz, 1H), 7.32 (d, J=9.0 Hz, 1H), 4.29 (dd, J=5.3, 3.7 Hz, 2H), 3.73 (dd, J=5.2, 3.7 Hz, 2H), 3.33 (s, 3H).

### Step-2: Synthesis of tert-butyl (S)-((1-(5-chloro-2-(2-methoxyethoxy)benzyl)pyrrolidin-3-yl)methyl) carbamate

### Procedure

To a stirred solution of 5-chloro-2-(2-methoxyethoxy)benzaldehyde (0.2 g, 0.93 mmol) in DCE (4.0 mL) at room temperature, tert-butyl (R)-(pyrrolidin-3-ylmethyl)carbamate hydrochloride (0.243 g, 1.02 mmol) was added. The reaction mixture was stirred at room temperature for 2 h. Sodium triacetoxyborohydride (0.591 g, 2.80 mmol) was added portion-wise to the reaction mixture at 0° C. The reaction was stirred at room temperature for 2 h. The progress of the reaction was monitored by TLC and LCMS analysis. After completion of the reaction, the reaction mixture was poured into a saturated solution of sodium bicarbonate (30 mL) and extracted with CH₂Cl₂ (3×20 mL). The combined organic fractions were washed with water (20 mL), dried over sodium sulphate and concentrated under reduced pressure. The crude material was purified by column chromatography (Aluminum oxide neutral; 50-55% ethyl acetate in hexane) to provide tert-butyl (S)-((1-(5-chloro-2-(2-methoxyethoxy)benzyl)pyrrolidin-3-yl)methyl)carbamate (0.32 g, 86.90% yield) as a light yellow liquid.

LCMS [ESI, M+1]: 398.8 (RT: 1.440 min, Purity: 96.40%),

¹H NMR (400 MHz, d₆-DMSO): δ 7.30 (d, J=2.6 Hz, 1H), 7.23 (dd, J=8.7, 2.6 Hz, 1H), 6.99 (d, J=8.7 Hz, 1H), 6.86 (s, 1H), 4.08 (dd, J=13.8, 9.2 Hz, 2H), 3.72-3.60 (m, 2H), 3.52 (d, J=16.5 Hz, 2H), 3.33 (d, J=3.3 Hz, 4H), 2.90 (t, J=6.0 Hz, 2H), 2.50-2.39 (m, 2H), 2.26 (dd, J=14.1, 8.9 Hz, 2H), 1.87-1.75 (m, 1H), 1.37 (s, 9H).

### Step-3: Synthesis of (S)-(1-(5-chloro-2-(2-methoxyethoxy)benzyl)pyrrolidin-3-yl)methanamine hydrochloride (Compound 54)

### Procedure

To a stirred solution of tert-butyl (S)-((1-(5-chloro-2-(2-methoxyethoxy)benzyl)pyrrolidin-3-yl)methyl)carbamate (0.32 g, 0.80 mmol) in CH₂Cl₂ (3.2 mL) at 0° C., 4M HCl in dioxane (1.6 mL, 5V) was added. The reaction mixture was stirred at room temperature for 1 h. The progress of the reaction was monitored by TLC and LCMS analysis. After completion of the reaction, the reaction mixture was concentrated under reduced pressure and the crude material purified by trituration with diethyl ether (2×20 mL) to provide (S)-(1-(5-chloro-2-(2-methoxyethoxy)benzyl)pyrrolidin-3-yl)methanamine hydrochloride (0.232, 87.87% yield) as a white solid.

LCMS [ESI, M+1]: 298.6 (RT: 0.763 min, Purity: 97.99%),

HPLC: RT: 3.747 min, Purity: 97.74%,

Chiral HPLC: RT: 2.23 min, Purity: 96.77%,

Instrument Name: Waters SFC Investigator

Chromatographic separation was conducted with Waters SFC Investigator system with 2998 PDA detector. The column used was CHIRALPAK® IG (250×4.6 mm; 5 ␣m) and the compounds were eluted with, Mobile Phase A: Liq·CO2, Mobile Phase B: 0.1% METHANOLIC AMMONIA in METHANOL with 65-35 composition method; with Flow rate=3 ml/min; Column oven temperature 40° C.; ABPR 130 bar, analysis time 6 min.

¹H NMR (400 MHz, CD₃OD): δ 7.63-7.55 (m, 1H), 7.49 (dd, J=8.9, 2.5 Hz, 1H), 7.17 (d, J=8.9 Hz, 1H), 4.55-4.41 (m, 2H), 4.37-4.22 (m, 2H), 3.91-3.82 (m, 2H), 3.82-3.55 (m, 2H), 3.46 (d, J=13.1 Hz, 3H), 3.44-3.37 (m, 1H), 3.23-3.05 (m, 2H), 3.02-2.70 (m, 1H), 2.38 (m, 1H), 2.11-1.80 (m, 1H).

### Experimental Protocol for Compound 55

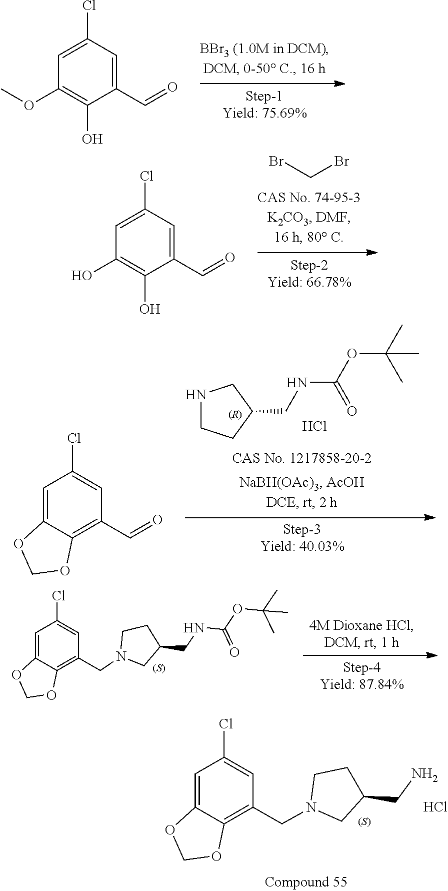

### Step-1: Synthesis of 5-chloro-2, 3-dihydroxybenzaldehyde

### Procedure

To a stirred solution of 5-chloro-2-hydroxy-3-methoxybenzaldehyde (0.5 g, 2.68 mmol) in CH₂Cl₂ (5 mL, 10V) at 0° C., boron tribromide 1M in CH₂Cl₂ (8 mL, 16.08 mmol) was slowly added. The reaction mixture was allowed to warm to room temperature and then heated to 50° C. for 16 h. The progress of the reaction was monitored by TLC and LCMS analysis. After completion of the reaction, the reaction mixture was concentrated under reduced pressure. The crude material was poured into ice-cold water (70 mL). A solid precipitate was formed which was isolated by filtration to provide 5-chloro-2, 3-dihydroxybenzaldehyde (0.352, 75.69% yield) as a yellow solid.

LCMS [ESI, M+1]: 172.04 (RT: 1.82 min, Purity: 88.97%).

### Step-2: Synthesis of 6-chlorobenzo[d][1,3]dioxole-4-carbaldehyde

### Procedure

To a stirred solution of 5-chloro-2,3-dihydroxybenzaldehyde (0.35 g, 2.02 mmol) in DMF (3 mL, 10V) at room temperature, dibromomethane (1.05 g, 6.08 mmol) and K₂CO₃ (0.83 g, 6.08 mmol) were added. The reaction mixture was heated to 80° C. for 16 h. The progress of the reaction was monitored by TLC and LCMS analysis. After completion of the reaction, the reaction mixture was poured into ice-cold water (50 mL). A solid precipitate was formed which was isolated by filtration to provide 6-chlorobenzo[d][1, 3] dioxole-4-carbaldehyde (0.25 g, 66.78% yield) as a yellow solid.

LCMS [ESI, M, M+2]: 184.01, 186.1 (RT: 6.977 min, Purity: 99.53%),

¹H NMR (400 MHz, d₆-DMSO): δ 9.98 (s, 1H), 7.38 (d, J=2.4 Hz, 1H), 7.28 (d, J=4 Hz, 1H), 6.28 (s, 2H).

### Step-3: Synthesis of tert-butyl (S)-((1-((6-chlorobenzo[d][1,3]dioxol-4-yl)methyl)pyrrolidin-3-yl)methyl)carbamate

### Procedure

To a stirred solution of 6-chlorobenzo[d][1,3]dioxole-4-carbaldehyde (0.25 g, 1.35 mmol) in DCE (5 mL, 20V) at room temperature, tert-butyl (R)-(pyrrolidin-3-ylmethyl)carbamate hydrochloride (0.32 g, 1.62 mmol) and acetic acid (0.005 mL, 0.05V) were added. The reaction mixture was stirred at room temperature for 2 h. Sodium triacetoxyborohydride (0.86 g, 4.07 mmol) was added portion-wise to the reaction mixture at 0° C. The reaction was stirred at room temperature for 2 h. The progress of the reaction was monitored by TLC and LCMS analysis. After completion of the reaction, the reaction mixture was poured into a saturated solution of NaHCO₃ (50 mL) and extracted with CH₂Cl₂ (3×30 mL). The combined organic fractions were dried over sodium sulphate and concentrated under reduced pressure. The crude material was purified by column chromatography (SiO₂; 30% ethyl acetate in hexane) to provide tert-butyl (S)-((1-((6-chlorobenzo[d][1,3]dioxol-4-yl)methyl)pyrrolidin-3-yl)methyl)carbamate (0.2 g, 40.03% yield) as a white solid.

LCMS [ESI, M+1]: 368.75 (RT: 1.440 min, Purity: 100%).

### Step-4: Synthesis of (S)-(1-((6-chlorobenzo[d][1,3]dioxol-4-yl)methyl)pyrrolidin-3-yl)methanamine hydrochloride (Compound 55)

### Procedure

To a stirred solution of tert-butyl (S)-((1-((6-chlorobenzo[d][1,3]dioxol-4-yl)methyl)pyrrolidin-3-yl)methyl)carbamate (0.2 gm, 0.54 mmol) in CH₂Cl₂ (2 mL, 10V) at 0° C., 4M HCl in dioxane (1 mL, 5V) was added. The reaction mixture was stirred at room temperature for 1 h. The progress of the reaction was monitored by TLC. After completion of the reaction, the reaction mixture was concentrated under reduced pressure and the crude material was purified by trituration with diethyl ether (3×15 mL) to provide (S)-(1-((6-chlorobenzo[d][1,3]dioxol-4-yl)methyl) pyrrolidin-3-yl)methanamine hydrochloride (0.12 g, 87.84% yield) as an off-white solid.

LCMS [ESI, M+1]: 268.69 (RT: 0.707 min, Purity: 100%),

HPLC: RT: 3.940 min, Purity: 100%,

Chiral HPLC: RT: 2.70 min, Purity: 100%,

Instrument Name: Waters SFC Investigator

Chromatographic separation was conducted with Waters SFC Investigator system with 2998 PDA detector. The column used was CHIRALPAC OD-H (250×4.6 mm 5 um) and the compounds were eluted with, Mobile Phase A: Liq·CO2, Mobile Phase B: 0.1% METHANOLIC AMMONIA in METHANOL-ACETONITRILE (50-50) with 70-30 composition method; with Flow rate=3 ml/min; Column oven temperature 40° C.; ABPR 130 bar, analysis time 6 min.

¹H NMR (400 MHz, CD₃OD): δ 7.12 (d, J=8 Hz, 1H), 7.02 (d, J=8 Hz, 1H), 6.16 (s, 2H), 4.42 (s, 2H), 3.84-3.72 (m, 2H), 3.55-3.42 (m, 2H), 3.20-3.15 (q, 2H), 2.92-2.77 (dd, 1H), 2.48-2.32 (m, 1H), 2.05-1.86 (m, 1H).

### Experimental Protocol for Compound 56

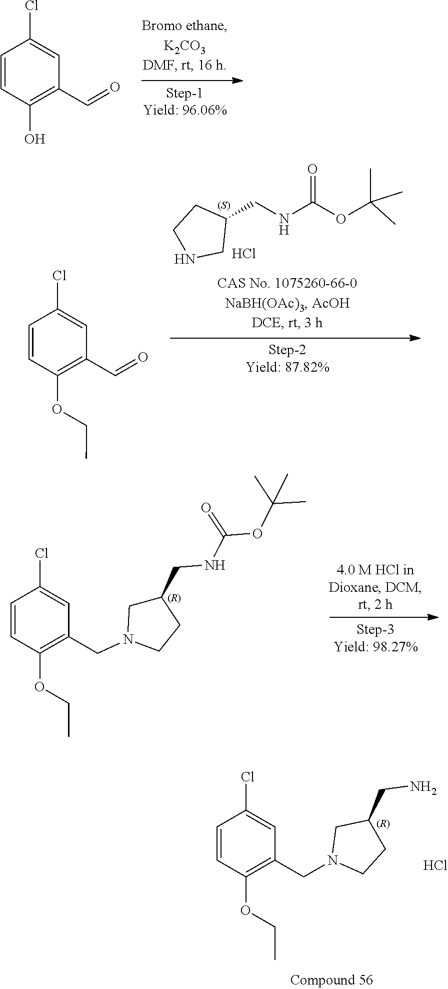

### Step-1: Synthesis of 5-chloro-2-ethoxybenzaldehyde

### Procedure

To a stirred solution of 5-chloro-2-hydroxybenzaldehyde (15.0 g, 95.84 mmol) in DMF (150 mL) at room temperature, potassium carbonate (33.09 g, 239.6 mmol) was added. The reaction mixture was stirred at room temperature for 1 h. Bromoethane (10.71 mL, 124.60 mmol) was added to the reaction mixture. The reaction mixture was stirred at room temperature for 16 h. The progress of the reaction was monitored by TLC. After completion of the reaction, the reaction mixture was poured into cold water (130 mL). The solid precipitate was isolated by filtration and dried under reduced pressure to provide 5-chloro-2-ethoxybenzaldehyde (17.0 g, 96.06% yield) as a white solid.

¹H NMR (400 MHz, CDCl₃): δ 10.45 (s, 1H), 7.80 (dd, J=8.9, 2.8 Hz, 1H), 7.50 (d, J=2.7 Hz, 1H), 6.96 (d, J=9.0 Hz, 1H), 4.19 (q, J=6.9 Hz, 2H), 1.50 (t, J=7.0 Hz, 3H).

### Step-2: Synthesis of tert-butyl (R)-((1-(5-chloro-2-ethoxybenzyl)pyrrolidin-3-yl)methyl) carbamate

### Procedure

To a stirred solution of 5-chloro-2-ethoxybenzaldehyde (4.0 g, 2.72 mmol) in DCE (80 mL) at room temperature, tert-butyl (S)-(pyrrolidin-3-ylmethyl)carbamate hydrochloride (5.50 g, 23.24 mmol) was added. The reaction mixture was stirred at room temperature for 1 h. Sodium triacetoxyborohydride (11.51 g, 54.3 mmol) was added portion-wise to the reaction mixture at 0° C. The reaction mixture was allowed to stir at room temperature for 3 h. The progress of the reaction was monitored by TLC and LCMS analysis. After completion of the reaction, the reaction mixture was poured into water (30 mL) and extracted with CH₂Cl₂ (3×100 mL). The combined organic fractions were washed with sat. NaHCO₃ solution (2×50 mL), dried over anhydrous sodium sulphate, and concentrated under reduced pressure. The crude material was purified by column chromatography (neutral alumina as stationary phase; 0-10% EtOAc/hexane) to provide tert-butyl (R)-((1-(5-chloro-2-ethoxybenzyl) pyrrolidin-3-yl)methyl) carbamate (7.0 g, 87.82% yield) as a yellow oil.

LCMS [ESI, M+1]: 368.9 (RT: 1.521 min, Purity: 100%),

Chiral HPLC: RT: 2.33 min, Purity: 100%

### Step-3: (R)-(1-(5-chloro-2-ethoxybenzyl)pyrrolidin-3-yl)methanamine hydrochloride (Compound 56)

### Procedure

To a stirred solution of tert-butyl (R)-((1-(5-chloro-2-ethoxybenzyl) pyrrolidin-3-yl)methyl) carbamate (7.0 g, 19.01 mmol) in CH₂Cl₂ (70.0 mL) at 0° C., 4.0 M HCl in dioxane (20 mL, 2V) was added. The reaction mixture was stirred at room temperature for 2 h. The progress of the reaction was monitored by TLC and LCMS analysis. After completion of the reaction, the reaction mixture was concentrated under reduced pressure. The crude material was purified by trituration using diethyl ether (50 mL) to provide (R)-(1-(5-chloro-2-ethoxybenzyl)pyrrolidin-3-yl)methanamine hydrochloride (5.7 g, 98.27% yield) as a white sticky solid.

LCMS [ESI, M+1]: 268.9 (RT: 0.727 min, Purity: 100%),

HPLC Purity: RT: 3.852 min, Purity: 100%,

Chiral HPLC: RT: 2.74 min, Purity: 100%,

Instrument Name: Waters SFC Investigator

Chromatographic separation was conducted with Waters SFC Investigator system with 2998 PDA detector. The column used was CHIRALPAK® IA (250×4.6 mm; 5 ␣m) and the compounds were eluted with, Mobile Phase A: Liq·CO2, Mobile Phase B: 0.1% METHANOLIC AMMONIA in METHANOL with 50-50 composition method; with Flow rate=3 ml/min; Column oven temperature 40° C.; ABPR 130 bar, analysis time 7 min.

¹H NMR (400 MHz, CD₃OD): δ 7.62 (dd, J=7.5, 2.6 Hz, 1H), 7.46 (ddd, J=8.9, 2.5, 1.2 Hz, 1H), 7.12 (d, J=8.9 Hz, 1H), 4.46 (td, J=12.8, 7.4 Hz, 2H), 4.21 (qd, J=6.9, 1.4 Hz, 2H), 3.82-3.63 (m, 2H), 3.63-3.37 (m, 2H), 3.17 (ddd, J=14.8, 11.5, 7.6 Hz, 2H), 3.04-2.80 (m, 1H), 2.42 (dddd, J=23.8, 21.6, 11.8, 5.3 Hz, 1H), 2.12-1.88 (m, 1H), 1.50 (t, J=7.0 Hz, 3H).

### Experimental Protocol for Compound 57

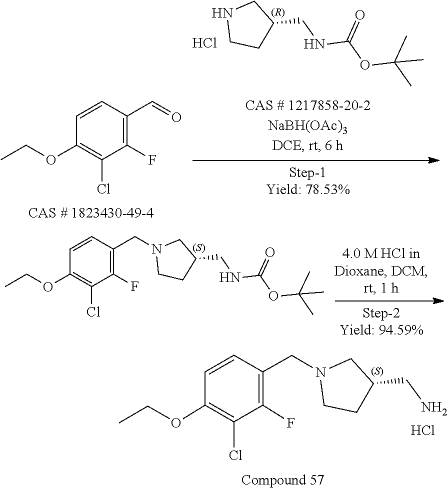

### Step-1: Synthesis of tert-butyl (S)-((1-(3-chloro-4-ethoxy-2-fluorobenzyl)pyrrolidin-3-yl)methyl) carbamate

To a stirred solution of 3-chloro-4-ethoxy-2-fluorobenzaldehyde (0.2 g, 0.99 mmol) in DCE (4 mL, 20V) at room temperature, tert-butyl (R)-(pyrrolidin-3-ylmethyl)carbamate hydrochloride (0.28 g, 1.18 mmol) was added. The reaction mixture was stirred at room temperature for 2 h. Sodium triacetoxyborohydride (0.62 g, 2.97 mmol) was added portion-wise to the reaction mixture at 0° C. The reaction mixture was allowed to stir at room temperature for 6 h. The progress of the reaction was monitored by TLC and LCMS analysis. After completion of the reaction, the reaction mixture was poured in a saturated solution of NaHCO₃ (50 mL) and extracted with CH₂Cl₂ (3×30 mL). The combined organic fractions were washed with a saturated brine solution (50 mL), dried over anhydrous sodium sulphate, and concentrated under reduced pressure. The crude material was purified by flash column chromatography (neutral alumina silica gel; 100% ethyl acetate) to provide tert-butyl (S)-((1-(3-chloro-4-ethoxy-2-fluorobenzyl)pyrrolidin-3-yl)methyl)carbamate (0.3 g, 78.53% yield) as a yellow oil.

LCMS [ESI, M+1]: 387.0 (RT: 1.572 min, Purity: 91.91%),

### Step-2: Synthesis of (S)-(1-(3-chloro-4-ethoxy-2-fluorobenzyl)pyrrolidin-3-yl)methanamine hydrochloride (Compound 57)

To a stirred solution of tert-butyl (S)-((1-(3-chloro-4-ethoxy-2-fluorobenzyl)pyrrolidin-3-yl)methyl)carbamate (0.3 g, 0.77 mmol) in CH₂Cl₂ (3 mL, 10V) at 0° C., 4.0 M HCl in dioxane (1.5 mL, 5V) was added. The reaction mixture was then stirred at room temperature for 1 h. The progress of the reaction was monitored by TLC and LCMS analysis. After completion of the reaction, the reaction mixture was concentrated under reduced pressure. The crude material was purified by trituration using n-pentane (2×10 mL) and diethyl ether (3×10 mL) to provide (S)-(1-(3-chloro-4-ethoxy-2-fluorobenzyl)pyrrolidin-3-yl)methanamine hydrochloride (0.21 g, 94.59% yield) as an off-white solid.

LCMS [ESI, M+1]: 287.0 (RT: 0.849 min, Purity: 98.11%)

Chiral HPLC: RT: 4.24 min, Purity: 98.57%

Instrument Name: Waters SFC Investigator

Chromatographic separation was conducted with Waters SFC Investigator system with 2998 PDA detector. The column used was CHIRALPAK® IG, (250×4.6 mm; 5 ␣m) and the compounds were eluted with, Mobile Phase A: Liq·CO2, Mobile Phase B: 0.1% Methanolic ammonia in Methanol: Acetonitrile (50-50) with Isocratic method (75:25); with Flow rate=4 ml/min; analysis time 12 min.

HPLC Purity: RT: 3.97 min, Purity: 98.62%

¹H NMR (400 MHz, CD₃OD): δ 8.13-7.28 (m, 1H), 7.19-6.63 (m, 1H), 4.51 (s, 2H), 4.21 (t, J=6.1 Hz, 2H), 3.87-3.61 (m, 1H), 3.57-3.39 (m, 2H), 3.19-2.99 (m, 3H), 2.76 (d, J=7.5 Hz, 1H), 2.50-2.29 (m, 1H), 2.06-1.87 (m, 1H), 1.48 (t, J=7.0 Hz, 3H).

### Experimental Protocol for Compound 58

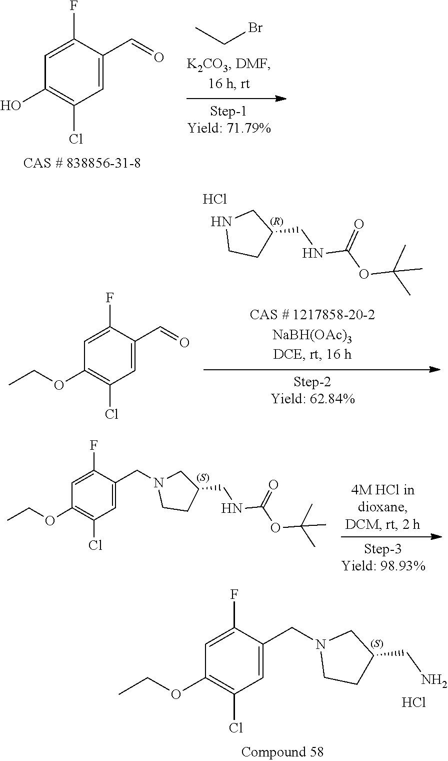

### Step-1: Synthesis of 5-chloro-4-ethoxy-2-fluorobenzaldehyde

To a stirred solution of 5-chloro-2-fluoro-4-hydroxybenzaldehyde (0.3 g, 1.718 mmol) in DMF (3 mL, 10V) at room temperature, K₂CO₃ (0.71 g, 5.15 mmol) was added. The reaction was stirred for 1 h. Then 1-bromoethane (0.37 g, 3.437 mmol) was added to the reaction mixture. The reaction mixture was stirred at room temperature for 16 h. The progress of the reaction was monitored by TLC. After completion of the reaction, the reaction mixture was poured into ice-cold water (70 mL) and extracted with ethyl acetate (3×30 mL). The combined organic fractions were washed with cold water, dried over sodium sulphate and concentrated under reduced pressure to provide 5-chloro-4-ethoxy-2-fluorobenzaldehyde (0.25 g, 71.79% yield) as a white solid.

¹H NMR (400 MHz, d₆-DMSO) δ 10.04 (s, 1H), 7.86-7.84 (d, J=7.6 Hz, 1H), 7.32-7.28 (d, J=12.8 Hz, 1H), 4.28-4.23 (q, J=4.0, 14.0 Hz, 2H), 1.41-1.37 (t, J=14.0 Hz, 3H).

### Step-2: Synthesis of tert-butyl (S)-((1-(5-chloro-4-ethoxy-2-fluorobenzyl)pyrrolidin-3-yl)methyl) carbamate

To stirred a solution of 5-chloro-4-ethoxy-2-fluorobenzaldehyde (0.25 g, 1.23 mmol) in DCE (2.5 mL, 10V) at room temperature, tert-butyl (R)-(pyrrolidin-3-ylmethyl)carbamate hydrochloride (0.32 g, 1.357 mmol) was added. The reaction mixture was stirred at room temperature for 2 h. Sodium triacetoxyborohydride (0.78 g, 3.701 mmol) was added to the reaction mixture portion-wise at 0° C. The reaction was stirred at room temperature for 1 h. The progress of the reaction was monitored by TLC and LCMS analysis. After completion of the reaction, the reaction mixture was poured into a saturated solution of sodium bicarbonate (50 mL) and extracted with CH₂Cl₂ (3×30 mL). The combined organic fractions were washed with water (30 mL), dried over sodium sulphate and concentrated under reduced pressure. The crude material was purified by column chromatography (SiO₂; 45-50% EtOAc in hexane) to provide tert-butyl (S)-((1-(5-chloro-4-ethoxy-2-fluorobenzyl)pyrrolidin-3-yl)methyl) carbamate (0.3 g, 62.84% yield) as a yellow sticky solid.

LCMS [ESI, M, M+2]: 387.0, 389.1 (RT: 1.492 min, Purity: 87.98%).

### Step-3: Synthesis of (S)-(1-(5-chloro-4-ethoxy-2-fluorobenzyl)pyrrolidin-3-yl)methanamine hydrochloride (Compound 58)

To a solution of tert-butyl (S)-((1-(5-chloro-4-ethoxy-2-fluorobenzyl)pyrrolidin-3-yl)methyl)carbamate (0.3 g, 0.777 mmol) in CH₂Cl₂ (3 mL, 10V) at room temperature, 4M hydrochloric acid in dioxane (1.2 mL, 5V) was added. The reaction mixture was stirred at room temperature for 2 h. The progress of the reaction was monitored by TLC and LCMS analysis. After completion of the reaction, the reaction mixture was concentrated under reduced pressure. The crude material was purified by trituration using diethyl ether (2×10 mL) and n-pentane (2×10 mL) to provide (S)-(1-(5-chloro-4-ethoxy-2-fluorobenzyl)pyrrolidin-3-yl)methanamine hydrochloride (0.25 g, 98.93% yield) as a white solid.

LCMS [ESI, M, M+2]: 287.0, 289.0 (RT: 0.831 min, Purity: 100.00%)

HPLC: RT: 3.81 min, Purity: 100.00%

Chiral HPLC: RT: 4.07 min, Purity: 96.33%

Instrument Name: Waters SFC Investigator

Chromatographic separation was conducted with Waters SFC Investigator system with 2998 PDA detector. The column used was CHIRALPAK® IB-N (250×4.6 mm; 5 ␣m) and the compounds were eluted with, Mobile Phase A: Liq·CO2, Mobile Phase B: 0.1% Methanolic ammonia in Methanol: Acetonitrile (50-50) with Gradient method; with Flow rate=4 ml/min; analysis time 8 min.

### Gradient

0 min>5% Co-solvent
5 min>50% Co-solvent
8 min>50% Co-solvent

¹H NMR (400 MHz, CD₃OD) δ 7.71 (m, 1H), 7.09-7.06 (d, J=11.6 Hz, 1H), 4.50 (s, 2H), 4.20-4.15 (qt, J=6.8, 13.6 Hz, 2H), 3.77-3.75 (m, 1H), 3.67-3.69 (m, 1H), 3.56-3.50 (m, 1H), 3.19-3.05 (m, 3H), 2.93-2.75 (m, 1H), 2.47-2.33 (m, 1H), 2.04-1.85 (m, 1H), 1.47 (t, J=14.0 Hz, 3H).

### Experimental Protocol for Compound 60

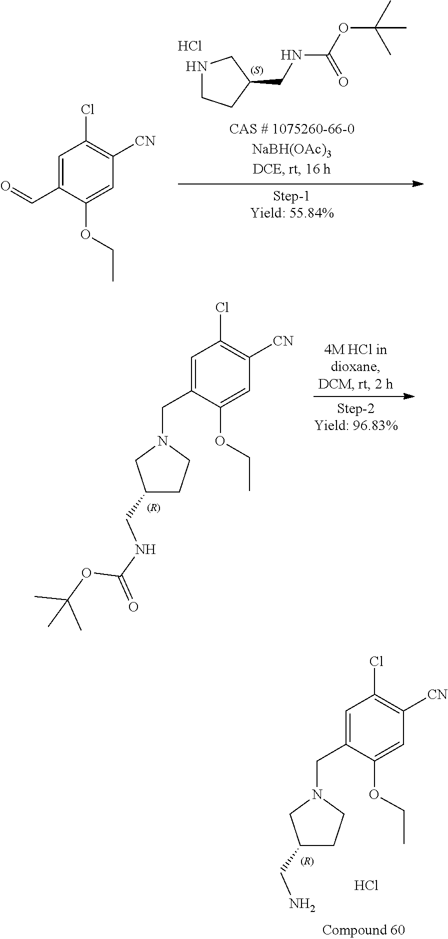

### Step-1: Synthesis of tert-butyl (R)-((1-(5-chloro-4-cyano-2-ethoxybenzyl)pyrrolidin-3-yl) methyl)carbamate

To a stirred solution of 2-chloro-5-ethoxy-4-formylbenzonitrile (0.2 g, 0.956 mmol) in DCE (2.0 mL) at room temperature, tert-butyl (S)-(pyrrolidin-3-ylmethyl)carbamate hydrochloride (0.23 g, 1.14 mmol) was added. The reaction mixture was stirred at room temperature for 2 h. Sodium triacetoxyborohydride (0.728 g, 2.868 mmol) was added portion-wise to the reaction mixture at 0° C. The reaction was stirred at room temperature for 13 h. The progress of the reaction was monitored by TLC and LCMS analysis. After completion of the reaction, the reaction mixture was poured into a saturated solution of sodium bicarbonate (20 mL) and extracted with CH₂Cl₂ (3×20 mL). The combined organic fractions were washed with water (10 mL), dried over sodium sulphate and concentrated under reduced pressure. The crude material was purified by column chromatography (aluminum oxide neutral; 10-20% ethyl acetate in hexane) to provide tert-butyl (R)-((1-(5-chloro-4-cyano-2-ethoxybenzyl)pyrrolidin-3-yl)methyl)carbamate (0.21 g, 55.84% yield) as a light yellow liquid.

LCMS [ESI, M, M+2]: 393.9, 395.7 (RT: 1.445 min, Purity: 98.54%)

### Step-2: Synthesis of (R)-4-((3-(aminomethyl)pyrrolidin-1-yl)methyl)-2-chloro-5-ethoxy benzonitrile hydrochloride (Compound 60)

To a stirred solution of tert-butyl (R)-((1-(5-chloro-4-cyano-2-ethoxybenzyl)pyrrolidin-3-yl)methyl)carbamate (0.18 g, 0.456 mmol) in CH₂Cl₂ (0.8 mL) at 0° C., 4M HCl in dioxane (0.8 mL) was added. The reaction mixture was stirred at room temperature for 2 h. The progress of the reaction was monitored by TLC and LCMS analysis. After completion of the reaction, the reaction mixture was concentrated under reduced pressure. The crude material was purified by trituration with diethyl ether (3×15 mL) to provide (R)-4-((3-(aminomethyl)pyrrolidin-1-yl)methyl)-2-chloro-5-ethoxybenzonitrile hydrochloride (0.13 g, 96.83% yield) as a white solid.

LCMS [ESI, M, M+2]: 293.7, 295.8 (RT: 0.814 min, Purity: 98.67%)

HPLC Purity: RT: 3.71 min, Purity: 98.8%

Chiral HPLC Purity: RT: 19.65 min, Purity: 100%

Instrument Name: Shimadzu LC-20 AD

Chromatographic separation was conducted with Shimadzu LC-20 AD system with DAD detector. The column used was CHIRALPAK® IH (250×4.6 mm; 5 ␣m) and the compounds were eluted with, Mobile Phase A: 0.1% Methanolic ammonia in n-Heptane, Mobile Phase B: 0.1% Methanolic ammonia in 2-Propanol with an isocratic method (90:10), with Flow rate=1 ml/min; analysis time 40 min.

¹H NMR (400 MHz, D₂O): δ 7.52 (s, 1H), 7.40 (s, 1H), 4.32 (s, 2H), 4.08-4.06 (m, 2H), 3.54-3.36 (m, 3H), 3.02-2.98 (m, 3H), 2.68 (bs, 1H), 2.24 (bs, 1H), 1.76 (bs, 1H), 1.28-1.27 (m 3H).

### Experimental Protocol for Compound 61

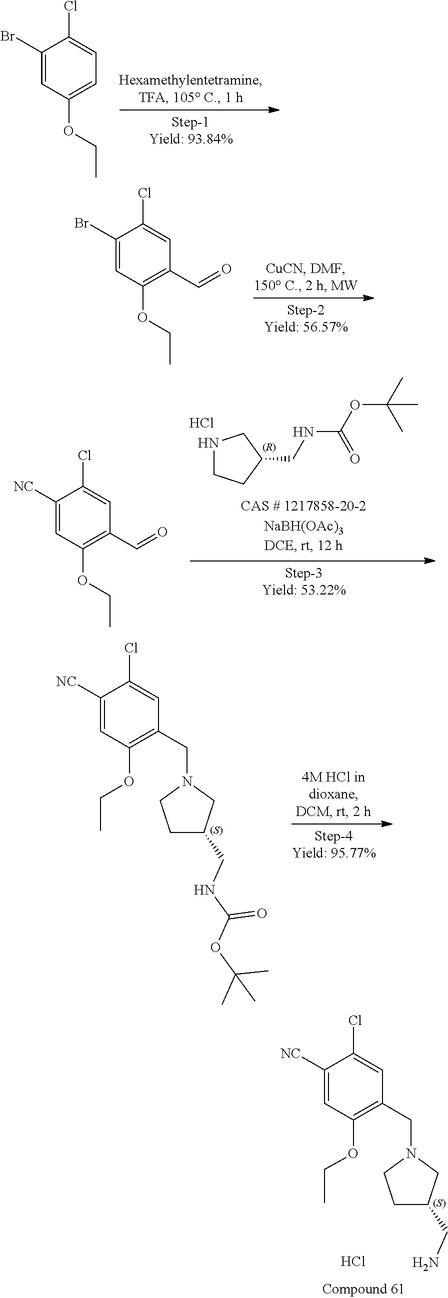

### Step-1: Synthesis of 4-bromo-5-chloro-2-ethoxybenzaldehyde

To a stirred solution of 2-bromo-1-chloro-4-ethoxybenzene (2.0 g, 8.49 mmol) in TFA (20 mL) at 0° C., hexamethylenetetramine (1.5 g, 1.10 mmol) was added. The reaction mixture was then stirred at 105° C. for 1 h. The progress of the reaction was monitored by TLC analysis. After completion of the reaction, the reaction mixture was poured into an ice-cold sat. solution of NaHCO₃ (50 mL) and extracted with ethyl acetate (3×50 mL). The combined the organic fractions were dried over sodium sulphate and concentrated under reduced pressure to provide 4-bromo-5-chloro-2-ethoxybenzaldehyde (2.1 g, 93.84% yield) as an off-white solid.

¹H NMR (400 MHz, d₆-DMSO): δ 10.26-10.22 (m, 1H), 7.76-7.75 (m, 1H), 7.69 (s, 1H), 4.25 (q, J=6.8 Hz, 2H), 1.38 (t, J=7.2 Hz, 3H).

### Step-2: Synthesis of 2-chloro-5-ethoxy-4-formylbenzonitrile

To a stirred solution of 4-bromo-5-chloro-2-ethoxybenzaldehyde (1.0 g, 3.80 mmol) in DMF (10 mL, 10V) at room temperature, CuCN (0.85 g, 9.50 mmol) was added. The reaction mixture was stirred at 150° C. for 2 h in a microwave. The progress of the reaction was monitored by TLC and LCMS analysis. After completion of the reaction, the reaction mixture was poured into cold water (100 mL) and extracted with ethyl acetate (3×50 mL). The combined organic fractions were washed with cold water (100 mL), dried over sodium sulphate and concentrated under reduced pressure. The crude material was purified by column chromatography (SiO₂; 10-15% ethyl acetate in hexane) to provide 2-chloro-5-ethoxy-4-formylbenzonitrile (0.45 g, 56.57% yield) as a white solid.

¹H NMR (400 MHz, CDCl₃) δ 10.47 (s, 1H), 7.91 (s, 1H), 7.29-7.28 (d, J=4.3 Hz, 1H), 4.20 (q, J=6.8 Hz, 2H), 1.54 (t, J=7.2 Hz, 3H).

### Step-3: Synthesis of tert-butyl (S)-((1-(5-chloro-4-cyano-2-ethoxybenzyl)pyrrolidin-3-yl)methyl) carbamate

To a stirred solution of 2-chloro-5-ethoxy-4-formylbenzonitrile (0.2 g, 0.956 mmol) in DCE (2.0 mL) at room temperature, tert-butyl (R)-(pyrrolidin-3-ylmethyl)carbamate hydrochloride (0.23 g, 1.148 mmol) was added. The reaction mixture was stirred at room temperature for 1 h. Sodium triacetoxyborohydride (0.728 g, 2.868 mmol) was added portion-wise to the reaction mixture at 0° C. The reaction mixture was stirred at room temperature for 11 h. The progress of the reaction was monitored by TLC and LCMS analysis. After completion of the reaction, the reaction mixture was poured into a saturated solution of sodium bicarbonate (30 mL) and extracted with CH₂Cl₂ (2×30 mL). The combined organic fractions were washed with water (20 mL), dried over sodium sulphate and concentrated under reduced pressure. The crude material was purified by column chromatography (aluminum oxide neutral; 10-20% ethyl acetate in hexane) to provide tert-butyl (S)-((1-(5-chloro-4-cyano-2-ethoxybenzyl)pyrrolidin-3-yl)methyl)carbamate (0.21 g, 53.22% yield) as a light yellow liquid.

LCMS [ESI, M, M+2]: 393.9, 395.7 (RT: 1.441 min, Purity: 97.0%).

### Step-4: Synthesis of (S)-4-((3-(aminomethyl)pyrrolidin-1-yl)methyl)-2-chloro-5-ethoxybenzonitrile hydrochloride (Compound 61)

To a stirred solution of tert-butyl (S)-((1-(5-chloro-4-cyano-2-ethoxybenzyl)pyrrolidin-3-yl)methyl)carbamate (0.21 g, 0.533 mmol) in CH₂Cl₂ (0.8 mL) at 0° C., 4M HCl in dioxane (0.8 mL) was added. The reaction mixture was stirred at room temperature for 2 h. The progress of the reaction was monitored by TLC and LCMS analysis. After completion of the reaction, the reaction mixture was concentrated under reduced pressure. The crude material was purified by trituration with diethyl ether (3×15 mL) to provide (S)-4-((3-(aminomethyl)pyrrolidin-1-yl)methyl)-2-chloro-5-ethoxybenzonitrile hydrochloride (0.15 g, 95.77% yield) as a white solid.

LCMS [ESI, M, M+2]: 293.8, 295.6 (RT: 0.801 min, Purity: 97.26%),

HPLC Purity: RT: 3.71 min, Purity: 100%

Chiral HPLC Purity: RT: 4.66 min, Purity: 95.71%

Instrument Name: Waters SFC Investigator

Chromatographic separation was conducted with Waters SFC Investigator system with 2998 PDA detector. The column used was CHIRALPAK® IG (250×4.6 mm, 5 ␣m) and the compounds were eluted with, Mobile Phase A: Liq·CO2, Mobile Phase B: 0.1% Methanolic ammonia in Methanol: Acetonitrile (50-50) with Gradient method; with Flow rate=4 ml/min; analysis time 9 min.

### Gradient

0 min>5% Co-solvent
5 min>50% Co-solvent
9 min>50% Co-solvent

¹H NMR (400 MHz, D₂O) δ 7.53 (s, 1H), 7.42 (s, 1H), 4.31 (s, 2H), 4.08 (s, 2H), 3.53-3.35 (m, 3H), 3.02 (s, 3H), 2.69 (bs, 1H), 2.24 (bs, 1H), 1.76 (bs, 1H), 1.30 (s, 3H).

### Experimental Protocol for Compound 63

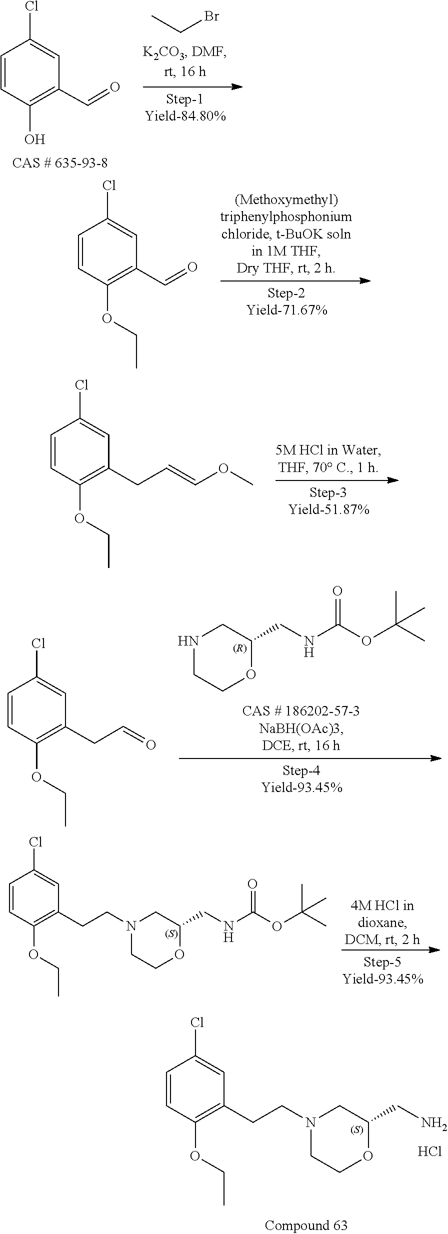

### Step-1: Synthesis of 5-chloro-2-ethoxybenzaldehyde

To a stirred solution of 5-chloro-2-hydroxybenzaldehyde (0.5 g, 3.19 mmol) in DMF (5 mL, 10V) at room temperature, potassium carbonate (1.3 g, 9.58 mmol) was added. The reaction mixture was stirred at room temperature for 1 h. Then ethyl bromide (0.3 ml, 4.78 mmol) was added drop-wise to the reaction mixture at 0° C. The reaction mixture was stirred at room temperature for 16 h. The progress of the reaction was monitored by TLC. After completion of the reaction, the reaction mixture was quenched by the addition of ice cold water (70 mL). A solid precipitate was formed which was isolated by filtration and dried under high vacuum to provide 5-chloro-2-ethoxybenzaldehyde (0.5 g, 84.80% yield) as a white solid.

LCMS [ESI, M+1]: 185.1 (RT: 2.598 min, Purity: 100%).

### Step-2: Synthesis of (E)-4-chloro-1-ethoxy-2-(2-methoxyvinyl) benzene

To a stirred solution of (methoxymethyl)triphenyl phosphonium chloride (2.3 g, 6.77 mmol) in dry THF (20 mL, 40V) at 0° C., t-BuOK solution in 1M THF (12.5 mL) was added. The reaction mixture was stirred at room temperature for 1 h. 5-chloro-2-ethoxybenzaldehyde (0.5 g, 2.70 mmol) in THF (1.0 mL) was added drop-wise to the reaction mixture at 0° C. The reaction was stirred at room temperature for 1 h. The progress of the reaction was monitored by TLC. After completion of the reaction, the reaction mixture was concentrated under reduced pressure. The crude material was purified by column chromatography (SiO₂; 100% in hexane) to provide (E)-4-chloro-1-ethoxy-2-(2-methoxyvinyl) benzene (0.44 g, 71.67% yield) as a light yellow liquid which was used directly used in the next step without further analysis.

### Step-3: Synthesis of 2-(5-chloro-2-ethoxyphenyl) acetaldehyde

To a stirred solution of (E)-4-chloro-1-ethoxy-2-(2-methoxyvinyl) benzene (0.44 g, 2.22 mmol) in dry THF (4.4 mL, 10V) at room temperature, 5M HCl (2.2 mL, 5V) was added. The reaction mixture was stirred at 70° C. for 1 h. The progress of the reaction was monitored by TLC analysis. After completion of the reaction, the reaction mixture was poured into a saturated solution of sodium bicarbonate (60 mL) and extracted with ethyl acetate (2×50 mL). The combined organic fractions were washed with water (2×15 mL), dried over sodium sulphate and concentrated under reduced pressure to provide 2-(5-chloro-2-ethoxyphenyl) acetaldehyde (0.2 g, 51.87% yield) as an off-white sticky solid which was used directly in the next step without analysis.

### Step-4: Synthesis of tert-butyl (S)-((4-(5-chloro-2-ethoxyphenethyl)morpholin-2-yl)methyl)carbamate

To a stirred solution of 2-(5-chloro-2-ethoxyphenyl)acetaldehyde (0.2 g, 0.50 mmol) in DCE (2.0 mL, 10V) at room temperature, tert-butyl (R)-(morpholin-2-ylmethyl)carbamate (0.129 g, 0.6 mmol) was added. The reaction mixture was stirred at room temperature for 1 h. Sodium triacetoxyborohydride (0.316 g, 1.5 mmol) was added portion-wise to the reaction mixture at 0° C. The reaction was stirred at room temperature for 16 h. The progress of the reaction was monitored by TLC and LCMS analysis. After completion of reaction, the reaction mixture was poured into a saturated solution of sodium bicarbonate (50 mL) and extracted with dichloromethane (3×25 mL). The combined organic fractions were washed with water (2×15 mL), dried over sodium sulphate and concentrated under reduced pressure. The crude material was purified by column chromatography (SiO₂; 42% ethyl acetate in hexane) to provide tert-butyl (S)-((4-(5-chloro-2-ethoxyphenethyl)morpholin-2-yl)methyl)carbamate (0.08 g, 19.92% yield) as a light yellow liquid.

LCMS [ESI, M+1]: 399.0 (RT: 1.628 min, Purity: 95.74%).

### Step-5: Synthesis of (S)-(4-(5-chloro-2-ethoxyphenethyl)morpholin-2-yl)methanamine hydrochloride (Compound 63)

To a stirred solution of tert-butyl (S)-((4-(5-chloro-2-ethoxyphenethyl)morpholin-2-yl)methyl)carbamate (0.08 g, 0.201 mmol) in CH₂Cl₂ (0.8 mL, 10V) at 0° C., 4M HCl in dioxane (0.4 mL, 5V) was added. The reaction mixture was stirred at room temperature for 2 h. After completion of the reaction, the reaction mixture was concentrated under reduced pressure and the crude material purified by trituration using diethyl ether (2×10 mL) to provide (S)-(4-(5-chloro-2-ethoxyphenethyl)morpholin-2-yl)methanamine hydrochloride (0.06 g, 93.45% yield) as a brown sticky solid.

LCMS [ESI, M, M+2]: 298.7, 300.6 (RT: 1.051 min, Purity: 94.66%),

HPLC: RT: 9.10 min, Purity: 91.40%,

Chiral HPLC: RT: 18.08 min, Purity: 96.32%,

Instrument Name: Shimadzu LC-20 AD

Chromatographic separation was conducted with Shimadzu LC-20 AD system with DAD detector. The column used was CHIRALPAK® IG (250×4.6 mm; 5 ␣m) and the compounds were eluted with, Mobile Phase A: 0.1% Methanolic ammonia in Methanol, Mobile Phase B: 0.1% Methanolic ammonia in Acetonitrile with an isocratic method (50:50) with Flow rate=1 ml/min; analysis time 40 min.

¹H NMR (400 MHz, CD₃OD) δ 7.28 (dt, J=8.7, 7.2 Hz, 2H), 6.99 (d, J=7.8 Hz, 1H), 4.29-4.18 (m, 2H), 4.12 (dd, J=13.9, 6.9 Hz, 2H), 4.04 (t, J=12.2 Hz, 1H), 3.72 (dd, J=23.9, 11.8 Hz, 2H), 3.40-3.36 (m, 2H), 3.22-3.22 (m, 2H), 3.13 (dd, J=17.5, 9.4 Hz, 2H), 3.05 (t, J=11.6 Hz, 2H), 1.47 (t, J=13.6 Hz, 3H).

### Experimental Protocol for Compound 64

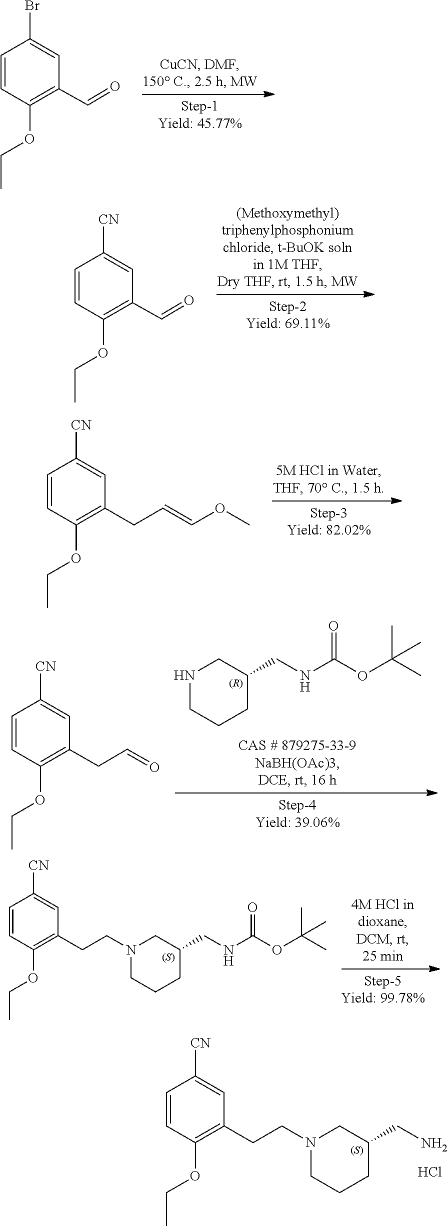

### Step-1: Synthesis of 4-ethoxy-3-formylbenzonitrile

To a stirred solution of 5-bromo-2-ethoxybenzaldehyde (lg, 4.36 mmol) in DMF (1 mL, 10V) at room temperature, CuCN (0.97 g, 10.91 mmol) was added. The reaction mixture was stirred at 150° C. in a microwave for 2.5 h. The progress of the reaction was monitored by TLC and LCMS analysis. After completion of the reaction, the reaction mixture was poured into cold water (100 mL) and extracted with ethyl acetate (3×70 mL). The combined organic fractions were washed with cold water (80 mL), dried over sodium sulphate and concentrated under reduced pressure. The crude material was purified by column chromatography (SiO₂; 20% ethyl acetate in hexane) to provide 4-ethoxy-3-formylbenzonitrile (0.35 g, 45.77% yield) as a white solid.

¹H NMR (400 MHz, d₆-DMSO): δ 10.32 (s, 1H), 8.10 (dd, J=2.4, 8.8 Hz, 1H), 8.06 (d, J=2.1 Hz, 1H), 7.43 (d, J=8.8 Hz, 1H), 4.30 (qt, J=6.8, 14.0 Hz, 2H), 1.41 (t, J=14.0 Hz, 3H).

### Step-2: Synthesis of (E)-4-ethoxy-3-(3-methoxyallyl)benzonitrile

To a stirred solution of (Methoxymethyl)triphenyl phosphine (0.3 g, 1.99 mmol) in dry THF (6 mL, 20V) at room temperature, t-BuOK in 1M THF (15 mL, 5V) was added. The reaction mixture was stirred at room temperature for 1 h. Then 4-ethoxy-3-formylbenzonitrile (0.35 g, 1.99 mmol) in dry THF (2.0 mL) was added drop-wise to the reaction mixture at room temperature. The reaction mixture was stirred at room temperature for 30 min. The progress of the reaction was monitored by TLC. After completion of the reaction, the reaction mixture was concentrated under reduced pressure. The crude material was purified by column chromatography (Aluminum oxide neutral; 0-2% ethyl acetate in hexane) to provide (E)-4-ethoxy-3-(3-methoxyallyl)benzonitrile (0.3 g, 69.11%) as a colorless oil, which was used in the next step of the reaction without further analysis.

### Step-3: Synthesis of 4-ethoxy-3-(2-oxoethyl)benzonitrile

To a stirred solution of (E)-4-ethoxy-3-(3-methoxyallyl)benzonitrile (0.3 g, 1.61 mmol) in THF (3.5 mL, 10V) at room temperature, 5M HCl (1.7 mL, 2.5M) was added. The reaction mixture was stirred at 70° C. for 1.5 h. The progress of the reaction was monitored by TLC. After completion of the reaction, the reaction mixture was quenched by the addition of a saturated solution of sodium bicarbonate (60 mL) and extracted with ethyl acetate (3×30 mL). The combined organic fractions were dried over sodium sulphate and concentrated under reduced pressure to provide 4-ethoxy-3-(2-oxoethyl)benzonitrile (0.25 g, 82.02%) as a light yellow liquid.

¹H NMR (400 MHz, CDCl₃) δ 9.74 (t, J=1.7 Hz, 1H), 7.62 (dd, J=11.2, 5.6 Hz, 1H), 7.44 (dd, J=23.5, 12.0 Hz, 1H), 6.95 (d, J=8.6 Hz, 1H), 4.18-4.09 (m, 2H), 3.71-3.70 (m, 2H), 1.47 (t, J=5.8 Hz, 3H),

### Step-4: Synthesis of tert-butyl (S)-((1-(5-cyano-2-ethoxyphenethyl)piperidin-3-yl)methyl)carbamate

To solution of 4-ethoxy-3-(2-oxoethyl)benzonitrile (0.25 g, 1.32 mmol) in DCE (2.5 mL, 10V) at room temperature, tert-butyl (R)-(piperidine-3-ylmethyl)carbamate (0.25 g, 1.45 mmol) was added. The reaction mixture was stirred at room temperature for 2 h. Sodium triacetoxyborohydride (0.61 g, 2.64 mmol) was added to the reaction mixture portion-wise at 0° C. The reaction was stirred at room temperature for 16 h. The progress of the reaction was monitored by TLC and LCMS analysis. After completion of the reaction, the reaction mixture was poured into a saturated solution of sodium bicarbonate (30 mL) and extracted with CH₂Cl₂ (3×15 mL). The combined organic fractions were washed with water (30 mL), dried over sodium sulphate and concentrated under reduced pressure. The crude material was purified by column chromatography (SiO₂; 8% MeOH in CH₂Cl₂) to provide tert-butyl (S)-((1-(5-cyano-2-ethoxyphenethyl)piperidin-3-yl)methyl)carbamate (0.2 g, 39.06% yield) as a pale yellow sticky solid.

LCMS [ESI, M+1]: 388.26 (RT: 1.464 min, Purity: 93.99%)

### Step-5: Synthesis of (S)-3-(2-(3-(aminomethyl)piperidin-1-yl)ethyl)-4-ethoxybenzonitrile hydrochloride (Compound 64)

To a stirred solution of tert-butyl (S)-((1-(5-cyano-2-ethoxyphenethyl)piperidin-3-yl)methyl)carbamate (0.2 g, 0.51 mmol) in CH₂Cl₂ (2 mL, 10V) at room temperature, 4M HCl in dioxane (5 mL, 5V) was added. The reaction mixture was stirred at room temperature for 25 min. The progress of the reaction was monitored by TLC and LCMS analysis. After completion of the reaction, the reaction mixture was concentrated under reduced pressure and the crude material purified by trituration using diethyl ether (3×10 mL) to provide (S)-3-(2-(3-(aminomethyl)piperidin-1-yl)ethyl)-4-ethoxybenzonitrile hydrochloride (0.15 g, 99.78%) as an off-white solid.

LCMS [ESI, M+1]: 287.94 (RT: 0.845 min, Purity: 96.65%)

HPLC: RT: 3.90, Purity: 98.14%

Chiral HPLC: RT: 9.324, Purity: 100.00%

Instrument Name: Shimadzu LC-20 AD

Chromatographic separation was conducted with Shimadzu LC-20 AD system with DAD detector. The column used was YMC CELLULOSE SC (250×4.6 mm; 5 ␣m) and the compounds were eluted with, Mobile Phase A: 0.1% Methanolic ammonia in n-Heptane, Mobile Phase B: 0.1% Methanolic ammonia in 2-Propanol:Acetonitrile (70:30) with an isocratic method (85:15), with Flow rate=1 ml/min; analysis time 25 min.

¹H NMR (400 MHz, CD₃OD): δ 7.70-7.67 (m, 2H), 7.16 (d, J=8.4 Hz, 1H), 4.23 (qt, J=7.2, 14 Hz, 2H), 3.74 (t, J=21.4 Hz, 2H), 3.20-3.16 (m, 2H), 3.04-3.01 (m, 2H), 2.94-2.87 (m, 2H), 2.36 (bs, 2H), 2.14-1.93 (m, 4H), 1.51 (t, J=32.4 Hz, 3H), 1.42-1.35 (m, 1H).

### Experimental Protocol for Compound 66

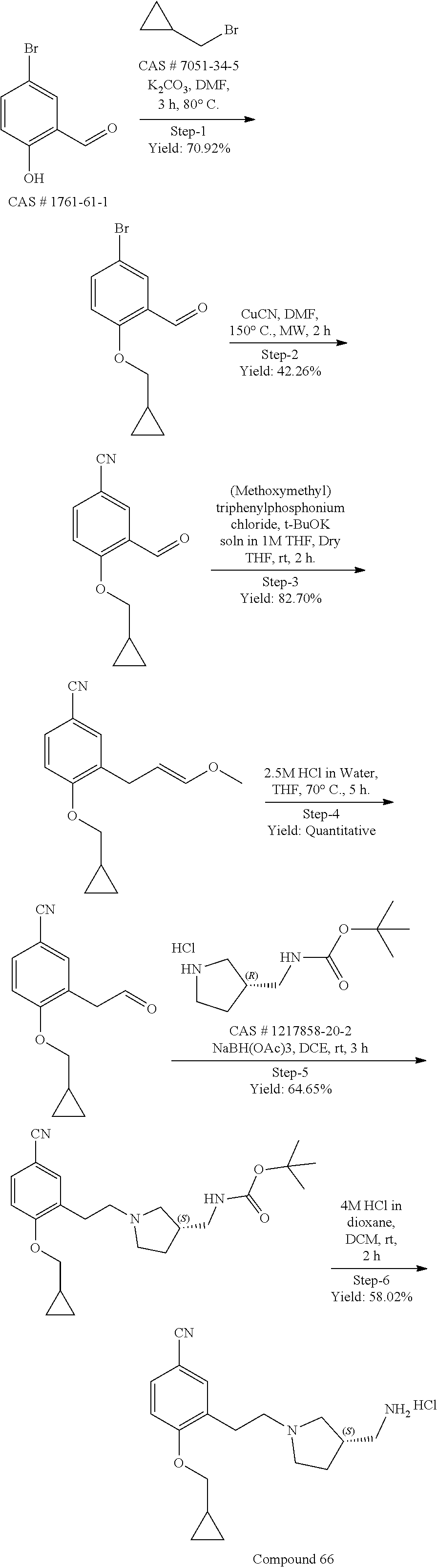

### Step-1: Synthesis of 5-bromo-2-(cyclopropylmethoxy)benzaldehyde

To a stirred solution of 5-bromo-2-hydroxybenzaldehyde (2.0 g, 9.94 mmol) in DMF (20 mL, 10V) at room temperature, K₂CO₃ (2.75 g, 19.90 mmol) was added. The reaction mixture was stirred for 1 h. Then (bromomethyl)cyclopropane (1.34 g, 9.94 mmol) was added to the reaction mixture. The reaction mixture was stirred at 80° C. for 3 h. The progress of the reaction was monitored by TLC. After completion of the reaction, the reaction mixture was poured into ice-cold water (200 mL) and extracted with ethyl acetate (3×80 mL). The combined organic fractions were washed with cold water (200 mL), dried over sodium sulphate and concentrated under reduced pressure to provide 5-bromo-2-(cyclopropylmethoxy)benzaldehyde (1.8 g, 70.92% yield) as a white solid.

¹H NMR (400 MHz, d₆-DMSO): δ 10.32 (s, 1H), 7.80 (dd, J=8.9, 2.0 Hz, 1H), 7.74 (d, J=2.1 Hz, 1H), 7.22 (d, J=8.9 Hz, 1H), 4.01 (d, J=7.0 Hz, 2H), 1.29-1.24 (m, 1H), 0.58 (dd, J=5.2, 12.8 Hz, 2H), 0.38 (d, J=4.8 Hz, 2H).

### Step-2: Synthesis of 4-(cyclopropylmethoxy)-3-formylbenzonitrile

To stirred solution of 5-bromo-2-(cyclopropylmethoxy)benzaldehyde (1.8 g, 7.05 mmol) in DMF (18 mL, 10V) at room temperature, copper cyanide (1.58 g, 17.64 mmol) was added. The reaction mixture was heated at 150° C. (microwave irradiation) for 2 h. The progress of the reaction was monitored by TLC and LCMS analysis. After completion of the reaction, the reaction mixture was poured into cold water (100 mL) and extracted with ethyl acetate (3×60 mL). The combined organic fractions were washed with cold water (100 mL), dried over sodium sulphate and concentrated under reduced pressure. The crude material was purified by flash column chromatography (SiO₂; 35-40% ethyl acetate in hexane) to provide 4-(cyclopropylmethoxy)-3-formylbenzonitrile (0.6 g, 42.26% yield) as a white solid.

LCMS [ESI, M+1]: 201.93 (RT: 1.867 min, Purity: 100%).

### Step-3: Synthesis of (E)-4-(cyclopropylmethoxy)-3-(3-methoxyallyl)benzonitrile

To a stirred solution of (Methoxymethyl)triphenyl phosphine (2.56 g, 7.45 mmol) in dry THF (26 mL, 10V) at room temperature, t-BuOK in 1M THF (14.92 mL, 14.91 mmol) was added. The reaction mixture was stirred at room temperature for 1 h. 4-(cyclopropylmethoxy)-3-formylbenzonitrile (0.6 g, 2.98 mmol) in dry THF (3 mL, 5 V) was added drop-wise into the reaction mixture at room temperature. The reaction mixture was stirred at room temperature for 2 h. The progress of the reaction was monitored by TLC. After completion of the reaction, the reaction mixture was concentrated under reduced pressure. The crude material was purified by flash column chromatography (Aluminum oxide neutral; 1-2% ethyl acetate in hexane) to provide (E)-4-(cyclopropylmethoxy)-3-(3-methoxyallyl)benzonitrile (0.6 g, 82.70%) as a colourless oil which was used in the next step without further analysis.

### Step-4: Synthesis of 4-(cyclopropylmethoxy)-3-(2-oxoethyl)benzonitrile

To a stirred solution of (E)-4-(cyclopropylmethoxy)-3-(3-methoxyallyl)benzonitrile (0.25 g, 2.577 mmol) in THF (2.5 mL, 10V) at room temperature, 2.5M HCl (2.5 mL, 10V) was added. The reaction mixture was stirred at 70° C. for 5 h. The progress of the reaction was monitored by TLC. After completion of the reaction, the reaction mixture was quenched by the addition of a saturated solution of sodium bicarbonate (30 mL) and extracted with ethyl acetate (2×20 mL). The combined organic fractions were dried over sodium sulphate and concentrated under reduced pressure to provide 4-(cyclopropylmethoxy)-3-(2-oxoethyl)benzonitrile (0.3 g, quantitative) as a light yellow liquid which was used in the next step without further analysis.

### Step-5: Synthesis of tert-butyl (S)-((1-(5-cyano-2-(cyclopropylmethoxy)phenethyl)pyrrolidin-3-yl)methyl) carbamate

To a stirred solution of 4-(cyclopropylmethoxy)-3-(2-oxoethyl)benzonitrile (0.2 g, 0.93 mmol) in DCE (4.0 mL, 20V) at room temperature, tert-butyl (R)-(pyrrolidin-3-ylmethyl)carbamate hydrochloride (0.22 g, 1.11 mmol) was added. The reaction mixture was stirred at room temperature for 2 h. Sodium triacetoxyborohydride (0.59 g, 2.7907 mmol) was added to the reaction mixture portion-wise at 0° C. The reaction was stirred at room temperature for 1 h. The progress of the reaction was monitored by TLC and LCMS analysis. After completion of the reaction, the reaction mixture was poured into a saturated solution of sodium bicarbonate (30 mL) and extracted with CH₂Cl₂ (3×15 mL). The combined organic fractions were washed with water (30 mL), dried over sodium sulphate and concentrated under reduced pressure. The crude material was purified by flash column chromatography (SiO₂; 50-55% ethyl acetate in hexane) to provide tert-butyl (S)-((1-(5-cyano-2-(cyclopropylmethoxy)phenethyl) pyrrolidin-3-yl)methyl)carbamate (0.24 g, 64.65% yield) as a yellow sticky solid.

LCMS [ESI, M+1]: 400.16 (RT: 1.538 min, Purity: 95.53%).

### Step-6: Synthesis of (S)-3-(2-(3-(aminomethyl)pyrrolidin-1-yl)ethyl)-4-(cyclopropylmethoxy)benzonitrile hydrochloride (Compound 66)

To a stirred solution of tert-butyl (S)-((1-(5-cyano-2-(cyclopropylmethoxy)phenethyl)pyrrolidin-3-yl)methyl)carbamate (0.23 g, 0.50 mmol) in CH₂Cl₂ (2.3 mL, 10V) at room temperature, 4M hydrochloric acid in dioxane (0.5 mL, 2V) was added. The reaction mixture was stirred at room temperature for 2 h. The progress of the reaction was monitored by TLC and LCMS analysis. After completion of the reaction, the reaction mixture was concentrated under reduced pressure and the crude material purified by trituration using diethyl ether (3×15 mL) to provide (S)-3-(2-(3-(aminomethyl)pyrrolidin-1-yl)ethyl)-4-(cyclopropylmethoxy)benzonitrile hydrochloride (0.07 g, 58.02%) as an off-white solid.

LCMS [ESI, M+1]: 299.99 (RT: 0.911 min, Purity: 99.48%).

HPLC: (RT: 4.29 min, Purity: 98.62%).

Chiral HPLC: (RT: 10.585 min, Purity: 95.85%).

Instrument Name: Shimadzu LC-20 AD

Chromatographic separation was conducted with Shimadzu LC-20 AD system with DAD detector. The column used was CHIRALPAK® IG (250×4.6 mm; 5 ␣m) and the compounds were eluted with, Mobile Phase A: n-Heptane, Mobile Phase B: 2-Propanol:Acetonitrile (70:30) with an isocratic method (70:30), with Flow rate=1 ml/min; analysis time 30 min.

¹H NMR (400 MHz, CD₃OD): δ 7.67 (d, J=6.4 Hz, 2H), 7.14 (d, J=9.0 Hz, 1H), 4.00 (dd, J=14.3, 8.0 Hz, 3H), 3.92-3.83 (m, 1H), 3.62-3.47 (m, 4H), 3.28-3.12 (m, 3H), 3.03 (t, J=10.9 Hz, 1H), 2.94-2.78 (m, 1H), 2.49-2.35 (m, 1H), 2.06-1.89 (m, 1H), 1.39 (m, 1H), 0.72-0.67 (q, J=12.8, 5.6 Hz, 2H), 0.46-0.41 (q, J=10, 4.4 Hz, 2H).

### Experimental Protocol for Compound 67

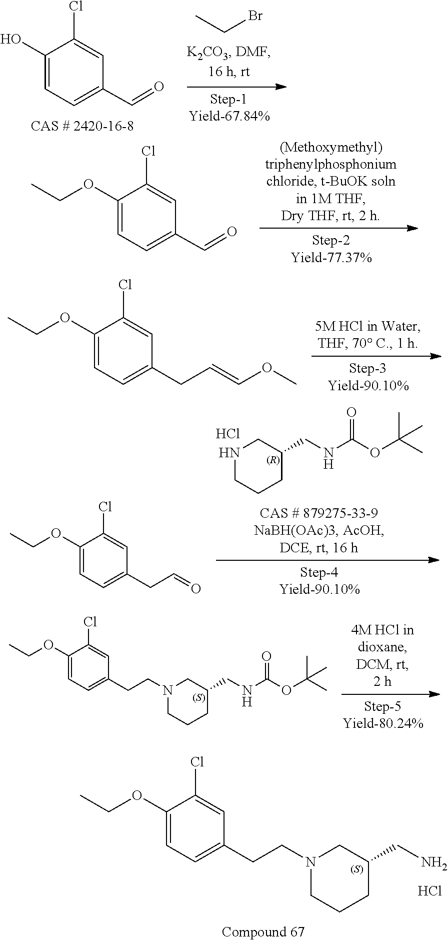

### Step-1: Synthesis of 3-chloro-4-ethoxybenzaldehyde

To a stirred solution of 3-chloro-4-hydroxybenzaldehyde, (0.5 g, 3.19 mmol) in DMF (5 mL, 10V) at room temperature, potassium carbonate (1.3 g, 9.58 mmol) was added. The reaction mixture was stirred at room temperature for 1 h. Ethyl bromide (0.3 ml, 4.78 mmol) was added drop-wise to the reaction mixture at 0° C. The reaction mixture was stirred at room temperature for 16 h. The progress of the reaction was monitored by TLC. After completion of the reaction, the reaction mixture was quenched by the addition of ice-cold water (30 mL). A precipitate was formed which was isolated by filtration to provide 3-chloro-4-ethoxybenzaldehyde (0.4 g, 67.84% yield) as an off-white solid.

LCMS [ESI, M, M+2]: 184.8, 186.7 (RT: 1.901 min, Purity: 98.94%).

### Step-2: Synthesis of (E)-2-chloro-1-ethoxy-4-(3-methoxyallyl)benzene

To a stirred solution of (methoxymethyl)triphenyl phosphonium chloride, (2.3 g, 5.46 mmol) in dry THF (23 mL, 40V) at 0° C., t-BuOK solution in 1M THF (12.5 mL) was added. The reaction mixture was stirred at room temperature for 1 h. Then 3-chloro-4-ethoxybenzaldehyde (0.4 g, 2.18 mmol) dissolved in THF (1 mL) was added drop-wise to the reaction mixture at 0° C. The reaction was stirred at room temperature for 30 min. The progress of the reaction was monitored by TLC. After completion of the reaction, the reaction mixture was concentrated under reduced pressure. The crude material was purified by column chromatography (SiO₂; 100% in hexane) to provide (E)-2-chloro-1-ethoxy-4-(3-methoxyallyl)benzene (0.38 g, 77.37% yield) as a light yellow liquid which was used directly in the next step without analysis.

### Step-3: Synthesis of 2-(3-chloro-4-ethoxyphenyl)acetaldehyde

To a stirred solution of (E)-2-chloro-1-ethoxy-4-(3-methoxyallyl)benzene (0.45 g, 1.98 mmol) in dry THF (4.5 mL, 10V) at room temperature, 5M HCl (2.2 mL, 5V) was added. The reaction mixture was stirred at 70° C. for 1 h. The progress of the reaction was monitored by TLC analysis. After completion of the reaction, the reaction mixture was poured into a saturated solution of sodium bicarbonate (70 mL) and extracted with ethyl acetate (2×50 mL). The combined organic fractions were washed with water (2×15 mL), dried over sodium sulphate and concentrated under reduced pressure to provide 2-(3-chloro-4-ethoxyphenyl)acetaldehyde (0.3 g, 71.37% yield) as an off-white sticky solid which was used directly in the next step without further analysis.

### Step-4: Synthesis of tert-butyl (S)-((1-(3-chloro-4-ethoxyphenethyl)piperidin-3-yl)methyl)carbamate

To a stirred solution of 2-(3-chloro-4-ethoxyphenyl)acetaldehyde (0.25 g, 1.51 mmol) in DCE (3.0 mL, 10V) at room temperature, tert-butyl (R)-(piperidin-3-ylmethyl)carbamate hydrochloride (0.38 g, 1.81 mmol) was added. The reaction mixture was stirred at room temperature for 1 h. Sodium triacetoxyborohydride (0.95 g, 4.54 mmol) was added portion-wise to the reaction mixture at 0° C. The reaction was stirred at room temperature for 16 h. The progress of the reaction was monitored by TLC and LCMS analysis. After completion of reaction, the reaction mixture was poured into a saturated solution of sodium bicarbonate (50 mL) and extracted with CH₂Cl₂ (3×50 mL). The combined organic fractions were washed with water (2×25 mL), dried over sodium sulphate and concentrated under reduced pressure. The crude material was purified by column chromatography (SiO₂; 20% ethyl acetate in hexane) to provide tert-butyl (S)-((1-(3-chloro-4-ethoxyphenethyl)piperidin-3-yl)methyl)carbamate (0.25 g, 90.10% yield) as a light yellow liquid.

LCMS [ESI, M, M+2]: 397.1, 398.9 (RT: 1.497 min, Purity: 89.3%).

### Step-5: Synthesis of (S)-(1-(3-chloro-4-ethoxyphenethyl)piperidin-3-yl)methanamine hydrochloride (Compound 67)

To a stirred solution of tert-butyl (S)-((1-(3-chloro-4-ethoxyphenethyl)piperidin-3-yl)methyl)carbamate (0.25 g, 0.63 mmol) in CH₂Cl₂ (2.5 mL, 10V) at 0° C., 4M HCl in Dioxane (1.5 mL, 5V) was added. The reaction mixture was stirred at room temperature for 2 h. After completion of reaction, the reaction mixture was concentrated under reduced pressure. The crude material was purified by reverse phase column chromatography (10% CH₃CN in water) to provide (S)-(1-(3-chloro-4-ethoxyphenethyl)piperidin-3-yl)methanamine hydrochloride (0.15 g, 80.24% yield) as an off-white solid.

LCMS [ESI, M, M+2]: 296.9, 298.7 (RT: 0.916 min, Purity: 99.33%),

HPLC: RT: 4.04 min, Purity: 95.43%,

Chiral HPLC: RT: 8.23, Purity: 100%

Instrument Name: Shimadzu LC-20 AD

Chromatographic separation was conducted with Shimadzu LC-20 AD system with DAD detector. The column used was YMC CELLULOSE SC (250×4.6 mm; 5 ␣m) and the compounds were eluted with, Mobile Phase A: 0.1% Methanolic ammonia in n-Heptane, Mobile Phase B: 0.1% Methanolic ammonia in 2-Propanol:Acetonitrile (70:30) with an isocratic method (55:45), with Flow rate=1 ml/min; analysis time 25 min.

¹H NMR (400 MHz, CD₃OD) δ 7.38-7.37 (m, 1H), 7.23-7.21 (m, 1H), 7.03 (d, J=8.4 Hz, 1H), 4.09 (qt, J=6.8, 13.6 Hz, 2H), 3.77-3.66 (m, 2H), 3.11-3.07 (m, 2H), 3.05-3.00 (m, 2H), 2.96-2.83 (m, 2H), 2.37-2.36 (m, 1H), 2.15-1.94 (m, 4H), 1.74 (dd, J=16.4, 8.4 Hz, 1H), 1.43 (t, J=7.0 Hz, 3H).

### Experimental Protocol for Compound 69

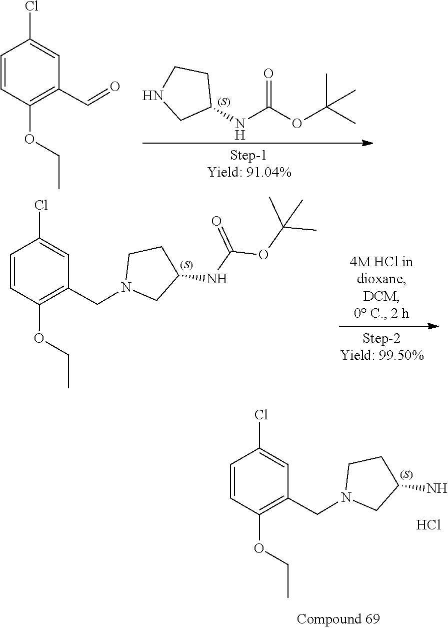

### Step-1: Synthesis of tert-butyl (S)-(1-(5-chloro-2-ethoxybenzyl)pyrrolidin-3-yl)carbamate

To a stirred solution of 5-chloro-2-ethoxybenzaldehyde (0.2 g, 1.08 mmol) in DCE (4.0 mL), tert-butyl (S)-pyrrolidin-3-ylcarbamate (0.242 g, 2.14 mmol) and AcOH (0.01 mL, 0.05V) were added. The reaction mixture was stirred at room temperature for 2 h. Sodium triacetoxyborohydride (0.686 g, 3.24 mmol) was added portion-wise to the reaction mixture at 0° C. The reaction was stirred at room temperature for 16 h. The progress of the reaction was monitored by TLC and LCMS analysis. After completion of the reaction, the reaction mixture was poured into a saturated solution of sodium bicarbonate (30 mL) and extracted with CH₂Cl₂ (2×20 mL). The combined organic fractions were washed with water (25 mL), dried over sodium sulphate and concentrated under reduced pressure to provide tert-butyl (S)-(1-(5-chloro-2-ethoxybenzyl)pyrrolidin-3-yl)carbamate (0.35 g, 91.04% yield) as a light yellow liquid.

LCMS [ESI, M, M+2]: 355.0, 356.7 (RT: 1.468 min, Purity: 97.66%)

Chiral HPLC: RT: 2.56 min, Purity: 100%.

### Step-2: Synthesis of (S)-1-(5-chloro-2-ethoxybenzyl)pyrrolidin-3-amine hydrochloride (Compound 69)

To a stirred solution of tert-butyl (S)-(1-(5-chloro-2-ethoxybenzyl)pyrrolidin-3-yl)carbamate (0.35 g, 0.986 mmol) in CH₂Cl₂ (3.5 mL) at 0° C., 4M HCl in Dioxane (1.75 mL, 5V) was added. The reaction mixture was stirred at 0° C. for 2 h. The progress of the reaction was monitored by TLC and LCMS analysis. After completion of reaction, the reaction mixture was concentrated under reduced pressure. The material was triturated with diethyl ether:CH₂Cl₂ (9:1, 2×10 mL) to provide (S)-1-(5-chloro-2-ethoxybenzyl)pyrrolidin-3-amine hydrochloride (0.25 g, 99.05% yield) as an off white sticky solid.

LCMS [ESI, M, M+2]: 254.8, 256.7 (RT: 0.785 min, Purity: 98.36%),

HPLC: RT: 3.90 min, Purity: 99.29%,

Chiral HPLC: RT: 2.65 min, Purity: 100%,

Instrument Name: Waters SFC Investigator

Chromatographic separation was conducted with Waters SFC Investigator system with 2998 PDA detector. The column used was CHIRALPAK® IG (250×4.6 mm; 5 ␣m) and the compounds were eluted with, Mobile Phase A: Liq·CO2, Mobile Phase B: 0.1% Methanolic ammonia in Methanol with Isocratic method (60:40); with Flow rate=3 ml/min; analysis time 5.5 min.

¹H NMR (400 MHz, CD₃OD): δ 7.59 (s, 1H), 7.49-7.46 (m, 1H), 7.13 (dd, J=8.4 Hz, 1H), 4.52 (d, J=32.2 Hz, 2H), 4.21 (qt, J=7.0 Hz, 3H), 4.18-3.96 (m, 1H), 3.79-3.69 (m, 3H), 3.47-3.44 (m, 1H), 2.74-2.55 (m, 1H), 2.42-2.23 (m, 1H), 1.51 (t, J=14.0 Hz, 3H).

### Experimental Protocol for Compound 70

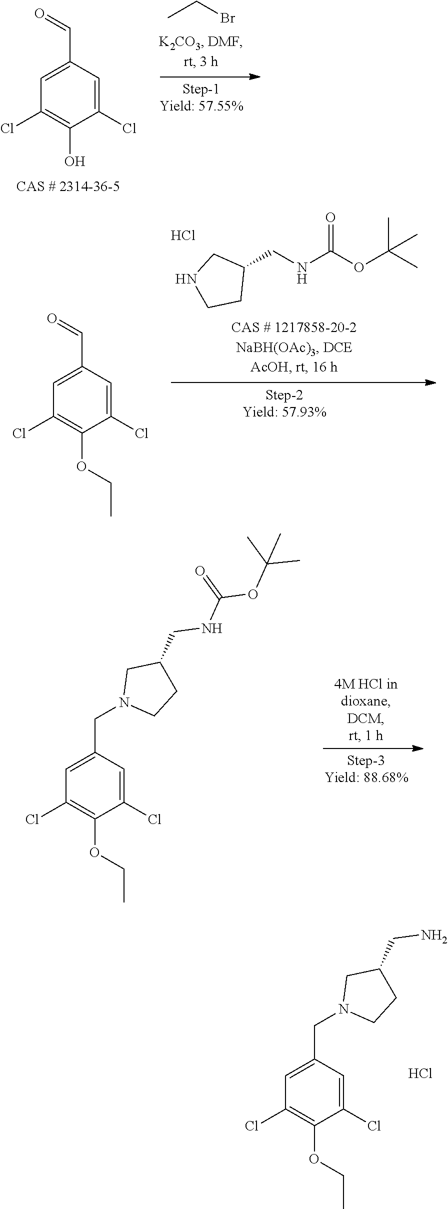

### Step-1: Synthesis of 3,5-dichloro-4-ethoxybenzaldehyde

To a stirred solution of 3,5-dichloro-4-hydroxybenzaldehyde (0.5 g, 2.61 mmol) in DMF (5.0 mL, 10V) at 0° C., K₂CO₃ (1.08 g, 7.83 mmol) was added. The reaction mixture was stirred for 30 min and allowed to warm to room temperature. Bromoethane (0.34 g, 3.14 mmol) was added and the reaction mixture was stirred at room temperature for 3 h.

The progress of the reaction was monitored by TLC and LCMS analysis. After completion of reaction, the reaction mixture was poured into cold water (30 mL) and extracted with EtOAc (3×20 mL). The combined organic fractions were washed with cold water (×2 times), dried over sodium sulfate and concentrated under reduced pressure. The crude material was purified by flash column chromatography (SiO2; 25% EtOAc in Hexane) to provide 3,5-dichloro-4-ethoxybenzaldehyde (0.33 g, 57.55% yield) as a light brown liquid.

¹H NMR (400 MHz, d₆-DMSO): δ 9.91 (s, 1H), 8.02 (s, 2H), 4.16 (q, J=14, 7.2 Hz, 2H), 1.40 (t, J=6.8 Hz, 3H).

### Step-2: Synthesis of tert-butyl (S)-((1-(3,5-dichloro-4-ethoxybenzyl)pyrrolidin-3-yl)methyl) carbamate

To a stirred solution of 3,5-dichloro-4-ethoxybenzaldehyde (0.3 g, 1.36 mmol) in DCE (3.0 mL, 10V) at room temperature, tert-butyl (R)-(pyrrolidin-3-ylmethyl)carbamate (0.35 g, 1.49 mmol) was added. After 2 h, NaBH(OAC)₃ (0.57 g, 2.73 mmol) was added to the reaction mixture at 0° C. The reaction mixture was stirred at rt for 14 h. The progress of the reaction was monitored by TLC and LCMS analysis. After completion of the reaction, the reaction mixture was poured into ice-cold water (15 mL) and extracted with CH₂Cl₂ (3×15 mL). The combined organic fractions were washed with cold water, dried over sodium sulfate and concentrated under reduced pressure. The crude material was purified by flash column chromatography (SiO₂; 25% EtOAc in Hexane) to provide tert-butyl (S)-((1-(3,5-dichloro-4-ethoxybenzyl)pyrrolidin-3-yl)methyl)carbamate (0.32 g, 57.93% yield) as a colourless liquid.

LCMS [ESI, M, M+2]: 403.0, 404.9 (RT: 1.623 min, Purity: 86.45%)

### Step-3: Synthesis of (S)-(1-(3,5-dichloro-4-ethoxybenzyl)pyrrolidin-3-yl)methanamine hydrochloride (Compound 70)

To a stirred solution of tert-butyl (S)-((1-(3,5-dichloro-4-ethoxybenzyl)pyrrolidin-3-yl)methyl)carbamate (0.3 g, 0.74 mmol) in CH₂Cl₂ (3.0 mL, 10V) at 0° C., 4M HCl in dioxane (1.5 mL, 5V) was added. The reaction mixture was stirred at room temperature for 1 h. The progress of the reaction was monitored by TLC and LCMS analysis. After completion of the reaction, the reaction mixture was concentrated under reduced pressure.

The crude material was purified by trituration with n-pentane and diethyl ether to provide (S)-(1-(3,5-dichloro-4-ethoxybenzyl)pyrrolidin-3-yl)methanamine hydrochloride (0.2 g, 88.68% yield) as an off-white solid.

LCMS [ESI, M, M+2]: 302.9, 304.8 (RT: 0.888 min, Purity: 99.27%)

HPLC: RT: 4.493 min, Purity: 95.14%

Chiral HPLC: RT: 3.76 min, Purity: 99.13%

Instrument Name: Waters SFC Investigator with PDA detector

Chromatographic separation was conducted with Waters SFC Investigator system with PDA detector. The column used was CHIRALPAK® IG (250×4.6 mm, 5 ␣m) and the compounds were eluted with Mobile Phase A: Liq·CO2, Mobile Phase B: 0.1% Methanolic Ammonia in Methanol:Acetonitrile (50:50) with Isocratic method (75:25); with Flow rate=4 ml/min; analysis time 12 min. 1H NMR (400 MHz, CD₃OD): δ 7.70 (s, 2H), 4.41 (bs, 2H), 4.14 (q, J=14.4, 7.2 Hz, 2H), 3.76-3.61 (m, 2H), 3.50-3.39 (m, 2H), 3.15 (d, J=7.2 Hz, 1H), 3.09 (bs, 1H), 2.92-2.74 (m, 1H), 2.46-2.34 (m, 1H), 2.03-1.86 (m, 1H), 1.46 (t, J=6.8 Hz, 3H).

### Experimental Protocol for Compound 71

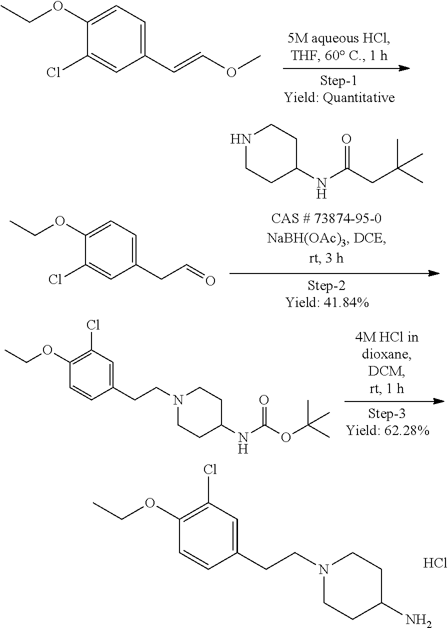

### Step-1: Synthesis of 2-(3-chloro-4-ethoxyphenyl)acetaldehyde

To a stirred solution of (E)-2-chloro-1-ethoxy-4-(2-methoxyvinyl)benzene (0.59 g, 2.79 mmol) in THF (5.9 ml, 10V) at room temperature, 5M aqueous HCl (2.9 mL, 5V) was added. The reaction mixture was stirred at 60° C. for 1 h. The progress of the reaction was monitored by TLC analysis. After completion of the reaction, the reaction mixture was quenched with saturated NaHCO₃ solution (50 mL) and extracted with EtOAc (2×50 mL). The combined organic fractions were dried over sodium sulfate and concentrated under reduced pressure to provide 2-(3-chloro-4-ethoxyphenyl)acetaldehyde (0.62 g, Quantitative yield) as a yellow sticky solid which was confirmed by TLC analysis using 2, 4-DNP stain and used directly in the next step.

### Step-2: Synthesis of tert-butyl (1-(3-chloro-4-ethoxyphenethyl)piperidin-4-yl)carbamate

To a stirred solution of 2-(3-chloro-4-ethoxyphenyl) acetaldehyde (0.62 g, 3.17 mmol) in DCE (6.2 mL, 10V) at room temperature, tert-butyl piperidin-4-ylcarbamate (0.76 g, 3.80 mmol) was added. The reaction mixture was stirred at room temperature for 1 h. Sodium triacetoxyborohydride (2.0 g, 9.51 mmol) was added portion-wise at 0° C. The reaction mixture was stirred at room temperature for 3 h. The progress of the reaction was monitored by TLC and LCMS analysis. After completion of the reaction, the reaction mixture was poured into a saturated solution of NaHCO₃ (50 mL) and extracted with CH₂Cl₂ (3×50 mL). The combined organic extracts were dried over sodium sulfate and concentrated under reduced pressure. The crude material was purified by flash column chromatography (SiO₂; 70% EtOAc in hexane) to provide tert-butyl (1-(3-chloro-4-ethoxyphenethyl)piperidin-4-yl) carbamate (0.5 g, 41.84% yield) as a yellow sticky solid.

LCMS [ES+, M, M+2]: 383.1, 385 (RT: 1.464 min, Purity: 93.01%)

### Step-3: Synthesis of 1-(3-chloro-4-ethoxyphenethyl)piperidin-4-amine hydrochloride (Compound 71)

To a stirred solution of tert-butyl (1-(3-chloro-4-ethoxyphenethyl)piperidin-4-yl) carbamate (0.5 g, 1.30 mmol) in CH₂Cl₂ (5.0 mL, 10V) at 0° C., 4M HCl in Dioxane (2.5 mL, 5V) was added. The reaction mixture was stirred at room temperature for 1 h. The progress of the reaction was monitored by TLC. After completion of the reaction, the reaction mixture was concentrated under reduced pressure. The crude material was purified by trituration using diethyl ether to provide 1-(3-chloro-4-ethoxyphenethyl)piperidin-4-amine hydrochloride (0.23 g, 62.28% yield) as an off-white solid.

LCMS [ESI, M, M+2]: 282.8, 284.6 (RT: 0.925 min, Purity: 98.44%),

HPLC: RT: 4.425 min, Purity: 95.0% ¹H NMR (400 MHz, CD₃OD): δ 7.28 (d, J=2 Hz, 1H), 7.12 (d, J=8.4 Hz, 1H), 7.01 (d, J=8.8 Hz, 1H), 4.08 (q, J=14, 7.2 Hz, 2H), 3.69-3.59 (m, 2H), 3.47-3.41 (m, 1H), 3.25-3.23 (m, 2H), 3.12-3.02 (m, 2H), 2.917 (t, J=8.0 Hz, 2H), 2.21 (d, J=13.2 Hz, 2H), 1.87-1.78 (m, 2H), 1.29 (t, J=7.2 Hz, 3H).

### Experimental Protocol for Compound 72

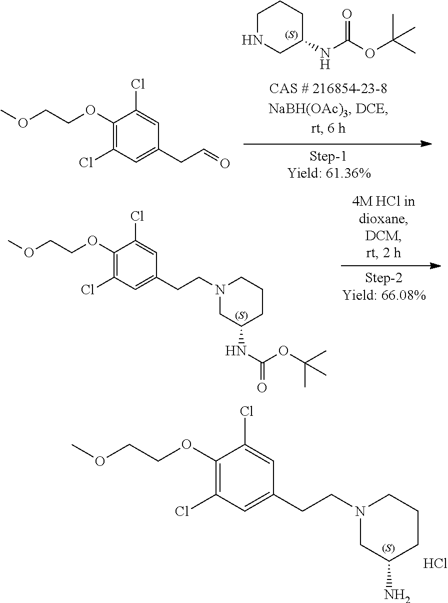

### Step-1: Synthesis of tert-butyl (S)-(1-(3,5-dichloro-4-(2-methoxyethoxy)phenethyl) piperidin-3-yl)carbamate

To a stirred solution of 2-(3,5-dichloro-4-(2-methoxyethoxy)phenyl)acetaldehyde (0.3 g, 1.14 mmol) in DCE (3 mL, 10V) at room temperature was added tert-butyl (s)-piperidin-3-ylcarbamate (0.27 g, 1.36 mmol) and acetic acid (0.015). The reaction mixture was stirred at room temperature for 1.5 h. Then sodium triacetoxyborohydride (0.72 g, 3.42 mmol) was added portion-wise at 0° C. and the reaction mixture was stirred at room temperature for 4.5 h. The progress of the reaction was monitored by TLC and LCMS analysis. After completion of reaction, the reaction mixture was poured into a saturated solution of NaHCO₃ (30 mL) and extracted with CH₂Cl₂ (3×20 mL). The combined organic fractions were dried over sodium sulfate and concentrated under reduced pressure. The crude material was purified by column chromatography (SiO₂; 2% MeOH in CH₂Cl₂) to provide tert-butyl (S)-(1-(3,5-dichloro-4-(2-methoxyethoxy)phenethyl)piperidin-3-yl)carbamate (0.31, 61.36% yield) as a viscous yellow liquid.

LCMS [M, M+2]: 447.0, 448.9 (RT: 1.438 min, Purity: 86.43%)

### Step-2: Synthesis of (S)-1-(3,5-dichloro-4-(2-methoxyethoxy)phenethyl)piperidin-3-amine hydrochloride (Compound 72)

To a stirred solution of tert-butyl (S)-(1-(3,5-dichloro-4-(2-methoxyethoxy)phenethyl) piperidin-3-yl)carbamate (0.31 g, 0.69 mmol) in CH₂Cl₂ (3.1 mL, 10V) at 0° C. was added 4M HCl in dioxane (1.5 mL, 5V). The reaction mixture was stirred at room temperature for 2 h. The progress of the reaction was monitored by TLC and LCMS analysis. After completion of the reaction, the reaction mixture was concentrated under reduced pressure and subjected to azeotropic distillation with CH₂Cl₂ (3×16 mL). The crude material was triturated using CH₂Cl₂ (3 mL) and diethyl ether (2×20 mL) to provide (S)-1-(3,5-dichloro-4-(2-methoxyethoxy)phenethyl) piperidin-3-amine hydrochloride (0.16 g, 66.08% yield) as an off-white sticky solid.

LCMS [ESI, M, M+2]: 347.3, 349.3 (RT: 3.015 min, Purity: 95.95%)

HPLC: RT: 4.818 min, Purity: 96.72%

Chiral HPLC Purity: RT: 8.38 min, Purity: 95.64%

Instrument Name: Waters SFC Investigator with PDA detector

Chromatographic separation was conducted with Waters SFC Investigator system with PDA detector. The column used was Chiral ICT (250 mm×4.6 mm, 5 μm) and the compounds were eluted with Mobile Phase A: Liq·CO2, Mobile Phase B: 0.1% Diethyl amine in 2-Propanol with Isocratic method (70:30); with Flow rate=4 ml/min; analysis time 15 min.

¹H NMR (400 MHz, CD₃OD): δ 7.27 (s, 2H), 4.13 (d, J=4.0 Hz, 2H), 3.74 (d, J=2.8 Hz, 3H), 3.55 (bs, 2H), 3.37-3.35 (m, 5H), 2.97 (t, J=8.8 Hz, 4H), 2.15-2.01 (m, 2H), 1.80-1.70 (m, 1H), 1.61-1.53 (m, 1H).

### Experimental Protocol for Compound 73

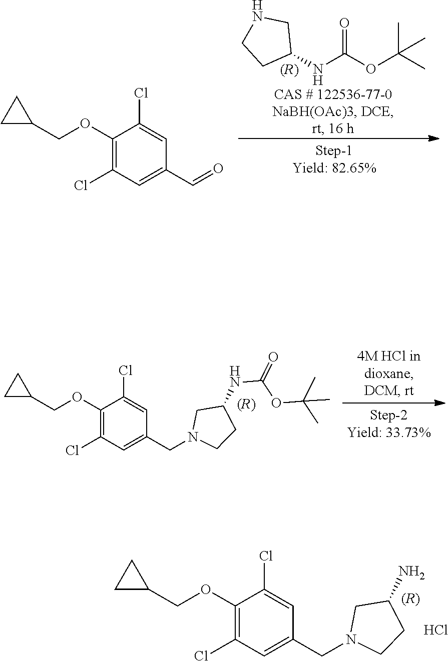

### Step-1: Synthesis of tert-butyl (R)-(1-(3,5-dichloro-4-(cyclopropylmethoxy)benzyl)pyrrolidin-3-yl)carbamate

To a stirred solution of 3,5-dichloro-4-(cyclopropylmethoxy)benzaldehyde (0.25 g, 1.02 mmol) in DCE (5.0 mL) at room temperature, tert-butyl (R)-pyrrolidin-3-ylcarbamate (0.22 g, 1.22 mmol) was added. After 1 h, sodium triacetoxyborohydride (0.64 g, 3.06 mmol) was added at 0° C. and the resulting reaction mixture was stirred at room temperature for 16 h. The progress of the reaction was monitored by TLC analysis. After completion of the reaction, the reaction mixture was poured into water (50 mL) and extracted with CH₂Cl₂ (3×30 mL). The combined organic fractions were dried over sodium sulfate and concentrated under reduced pressure. The crude material was purified by reverse phase column chromatography (75% CH₃CN in water) to provide tert-butyl (R)-(1-(3,5-dichloro-4-(cyclopropylmethoxy)benzyl)pyrrolidin-3-yl)carbamate (0.35 g, 82.65%) as a yellow oil.

LCMS [ESI, M, M+2]: 415.0, 416.9 (RT: 1.719 min, Purity: 100%),

### Step-2: Synthesis of (R)-1-(3,5-dichloro-4-(cyclopropylmethoxy)benzyl)pyrrolidin-3-amine hydrochloride (Compound 73)

To a stirred solution tert-butyl (R)-(1-(3,5-dichloro-4-(cyclopropylmethoxy)benzyl)pyrrolidin-3-yl)carbamate (0.35 g, 0.84 mmol) in CH₂C₁₂ (4 mL) at 0° C., 4M HCl in Dioxane (2.0 mL) was added. The reaction mixture was stirred at room temperature for 1 h. The progress of the reaction was monitored by TLC analysis. After completion of the reaction, the reaction mixture was concentrated under reduced pressure and triturate with diethyl ether. The crude material was purified by reverse phase column chromatography (80:20 CH₃CN:H₂O containing 0.05% aq. HCl) to provide (R)-1-(3,5-dichloro-4-(cyclopropylmethoxy)benzyl)pyrrolidin-3-amine hydrochloride (0.1 g, 33.73% yield) as a white sticky solid.

LCMS [ESI, M, M+2]: 315.2, 317.2 (RT: 3.604 min, Purity: 99.05%),

Chiral HPLC: RT: 5.84 min, Purity: 100%

Instrument Name: Waters SFC Investigator with PDA detector

Chromatographic separation was conducted with Waters SFC Investigator system with PDA detector. The column used was Chiral ICT (250 mm×4.6 mm, 5 μm) and the compounds were eluted with, Mobile Phase A: Liq·CO2, Mobile Phase B: 0.1% Diethyl amine in 2-Propanol with Isocratic method (50:50); with Flow rate=4 ml/min; analysis time 12 min.

¹H NMR (400 MHz, D₂O): δ 7.47 (s, 2H), 4.33 (s, 2H), 4.13-4.10 (m, 1H), 3.88 (d, J=7.6 Hz, 3H), 3.79 (t, J=12 Hz, 1H), 3.49-3.43 (m, 3H), 2.56-2.51 (m, 1H), 2.15-2.09 (m, 1H), 1.30-1.23 (m, 1H), 0.54-0.49 (m, 2H), 0.26-0.22 (m, 2H).

### Experimental Protocol for Compound 74

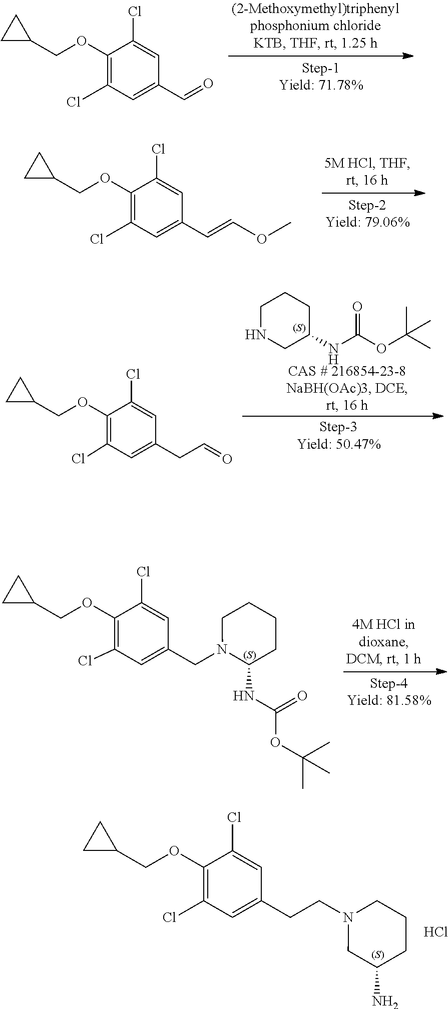

### Step-1: Synthesis of (E)-1,3-dichloro-2-(cyclopropylmethoxy)-5-(2-methoxyvinyl)benzene

To a stirred solution of (2-Methoxymethyl)triphenyl phosphoniumchloride (1.39 g, 4.07 mmol) in anhydrous THF (8 mL, 20V) at 0° C., potassium tert-butoxide 1M in THF (8.1 mL, 8.159 mmol) was added. After 1 h, 3,5-dichloro-4-(cyclopropylmethoxy) benzaldehyde (0.4 g, 1.63 mmol) was added at 0° C. Then the reaction mixture was stirred at room temperature for 15 min. The progress of the reaction was monitored by TLC. After completion of the reaction, the reaction mixture was diluted with EtOAc (15 mL) and filtered through Celite®. The solution was concentrated under reduced pressure and the crude material purified by flash column chromatography (SiO₂; 1% EtOAc in hexane) to provide (E)-1,3-dichloro-2-(cyclopropylmethoxy)-5-(2-methoxyvinyl)benzene (0.32 g, 71.78% yield) as a white sticky liquid.

### Step-2: Synthesis of 2-(3,5-dichloro-4-(cyclopropylmethoxy)phenyl)acetaldehyde

To a stirred solution of (E)-1,3-dichloro-2-(cyclopropylmethoxy)-5-(2-methoxyvinyl)benzene_(0.32 g, 1.17 mmol) in THF (3.2 mL, 10V) at room temperature, 5M aqueous HCl (1.6 mL, 5V) was added. The the reaction mixture was stirred at room temperature for 16 h. The progress of the reaction was monitored by TLC analysis. After completion of the reaction, the reaction mixture was quenched by addition into saturated NaHCO₃ solution (15 mL) and extracted with EtOAc (2×15 mL). The combined organic fractions were dried over sodium sulfate and concentrated under reduced pressure to provide 2-(3,5-dichloro-4-(cyclopropylmethoxy)phenyl)acetaldehyde (0.24 g, 79.06% yield) as a white sticky solid which was used directly in the next step of the synthesis.

### Step-3: Synthesis of tert-butyl (S)-(1-(3,5-dichloro-4-(cyclopropylmethoxy)phenethyl) piperidin-3-yl)carbamate

To a solution of 2-(3,5-dichloro-4-(cyclopropylmethoxy)phenyl)acetaldehyde (0.22 g, 0.84 mmol) added in DCE (2.2 mL, 10V) at room temperature, tert-butyl (S)-piperidin-3-ylcarbamate (0.2 g, 1.01 mmol) was added. After 2 h, NaBH(OAc)₃ (0.53 g, 2.54 mmol) was added at room temperature and the reaction mixture was stirred at room temperature for 16 h. The progress of the reaction was monitored by TLC analysis. After completion of the reaction, the reaction mixture was poured into water (60 mL) and extracted with CH₂Cl₂ (2×60 mL). The combined organic fractions were dried over sodium sulfate and concentrated under reduced pressure. The crude material was purified by reverse phase flash column chromatography (82% MeCN in water) to provide tert-butyl (S)-(1-(3,5-dichloro-4-(cyclopropylmethoxy)phenethyl)piperidin-3-yl)carbamate (0.19 g, 50.47% yield) as a yellow sticky solid.

LCMS [ESI, M, M+2]: 443.0, 444.9 (RT: 1.774 min, Purity: 99.48%)

### Step-4: Synthesis of (S)-1-(3,5-dichloro-4-(cyclopropylmethoxy)phenethyl)piperidin-3-amine hydrochloride (Compound 74)

To a stirred solution of tert-butyl (S)-(1-(3,5-dichloro-4-(cyclopropylmethoxy)phenethyl) piperidin-3-yl)carbamate (0.19 g, 0.42 mmol) in CH₂Cl₂ (2 mL) at 0° C., 4M HCl in Dioxane (0.95 mL, 5V) was added. The reaction mixture was stirred at room temperature for 1 h. The progress of the reaction was monitored by TLC. After completion of the reaction, the reaction mixture was concentrated under reduced pressure and azeotroped with CH₂Cl₂ (3×10 mL). The crude material was purified by trituration using diethyl ether (12 mL): n-pentane (5 mL) to provide (S)-1-(3,5-dichloro-4-(cyclopropylmethoxy)phenethyl)piperidin-3-amine hydrochloride (0.12 g, 81.58%) as a yellow solid.

LCMS [ESI, M+2]: 344.8 (RT: 1.197 min, Purity: 100%),

HPLC: RT: 3.82 min, Purity: 100%

Chiral HPLC: RT: 6.47 min, Purity: 99.33%

Instrument Name: Waters SFC Investigator

Chromatographic separation was conducted with Waters SFC Investigator system with 2998 PDA detector. The column used was (Chiral ICT 250×4.6 mm, 5 μm) and the compounds were eluted with, Mobile Phase A: Liq·CO2, Mobile Phase B: 0.1% Diethyl amine in 2-Propanol with Isocratic method (70:30); with Flow rate=4 ml/min; analysis time 12 min.

¹H NMR (400 MHz, D₂O): δ 7.26 (s, 2H), 3.82 (d, J=7.6 Hz, 2H), 3.76 (d, J=7.6 Hz, 1H) 3.59-3.54 (m, 2H), 3.37-3.33 (m, 2H), 2.98-2.94 (m, 4H), 2.14 (d, J=13.6 Hz, 1H), 2.04 (d, J=14.8, 1H), 1.80-1.69 (m, 1H), 1.62-1.54 (m, 1H), 1.27-1.21 (m, 1H), 0.53-0.50 (m, 2H), 0.25-0.23 (m, 2H).

### Experimental Protocol for Compound 75

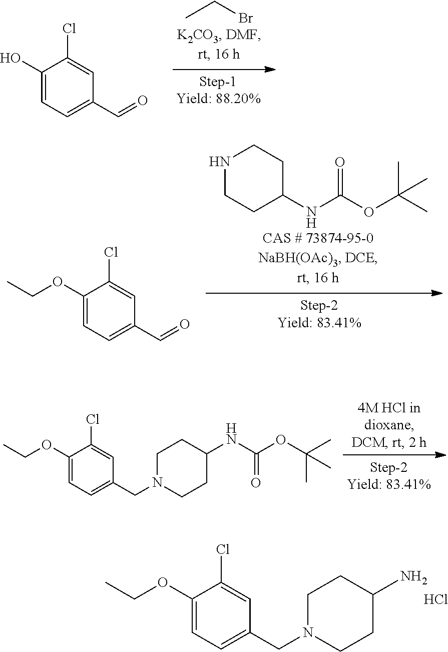

### Step-1: Synthesis of 3-chloro-4-ethoxybenzaldehyde

To a stirred solution of 3-chloro-4-hydroxybenzaldehyde (5.0 g, 31.9 mmol) in DMF (50 mL) at room temperature, K₂CO₃ (13.24 g, 95.8 mmol) was added. After 25 min, ethyl bromide (4.17 g, 38.3 mmol) was added dropwise wise at room temperature and the reaction mixture was stirred at room temperature for 16 h. The progress of the reaction was monitored by TLC analysis. After completion of the reaction, the reaction mixture was filtered and the solid material dried under reduced pressure to provide 3-chloro-4-ethoxybenzaldehyde (5.2 g, 88.20% yield) as an off-white solid.

¹H NMR (400 MHz, d₆-DMSO): δ 9.86 (s, 1H), 7.94 (d, J=2 Hz, 1H), 7.88 (dd, J=8.4, 2 Hz, 1H), 7.35 (d, J=8.4 Hz, 1H), 4.25 (q, J=14, 7.2 Hz, 2H), 1.39 (t, J=7.2 Hz, 3H)

### Step-2: Synthesis of tert-butyl (1-(3-chloro-4-ethoxybenzyl)piperidin-4-yl)carbamate

To a stirred solution of 3-chloro-4-ethoxybenzaldehyde (0.3 g, 1.62 mmol) in DCE (3 mL) at room temperature, tert-butyl piperidin-4-ylcarbamate (0.39 g, 1.94 mmol) was added. After 1.5 h, NaBH(OAc)₃ (1.03 g, 4.87 mol) was added portion-wise at 0° C. and the reaction mixture was stirred at room temperature for 16 h. The progress of the reaction was monitored by TLC analysis. After completion of the reaction, the reaction mixture was diluted with water (50 mL) and extracted with CH₂Cl₂ (2×50 mL). The combined organic fractions were dried over sodium sulfate and concentrated under reduced pressure. The crude material was purified by normal phase column chromatography (6% MeOH in CH₂Cl₂) to provide tert-butyl (1-(3-chloro-4-ethoxybenzyl)piperidin-4-yl)carbamate (0.5 g, 83.41% yield) as an off-white sticky liquid.

LCMS [ESI, M, M+2]: 369.1, 371.0 (RT: 1.397 min, Purity: 99.73%),

### Step-3: Synthesis of 1-(3-chloro-4-ethoxybenzyl)piperidin-4-amine hydrochloride (Compound 75)

To a stirred solution of tert-butyl (1-(3-chloro-4-ethoxybenzyl)piperidin-4-yl)carbamate (0.5 g, 0.1.35 mmol) in CH₂Cl₂ (5 mL) at 0° C., 4M HCl in Dioxane (2.5 mL, 5V) was added. The reaction mixture was stirred at room temperature for 2 h. The progress of the reaction was monitored by TLC analysis. After completion of the reaction, the reaction mixture was concentrated under reduced pressure. The crude material was purified by trituration using diethyl ether (2×10 mL) to provide 1-(3-chloro-4-ethoxybenzyl)piperidin-4-amine hydrochloride (0.39 g, 95% yield) as a white solid.

LCMS [ESI, M, M+2]: 268.9, 270.7 (RT: 0.815 min, Purity: 100%),

HPLC: RT: 4.273 min, Purity: 99.65%,

¹H NMR (400 MHz, D₂O): δ 7.44 (s, 1H), 7.28 (d, J=8.8 Hz, 1H), 7.08 (d, J=8.8 Hz, 1H), 4.13-4.09 (m, 4H), 3.47-3.38 (m, 3H), 3.09-2.98 (m, 2H), 2.18 (d, J=13.2 Hz, 2H), 1.79-1.76 (m, 2H), 1.31 (t, J=13.2 Hz, 3H).

### Experimental Protocol for Compound 76

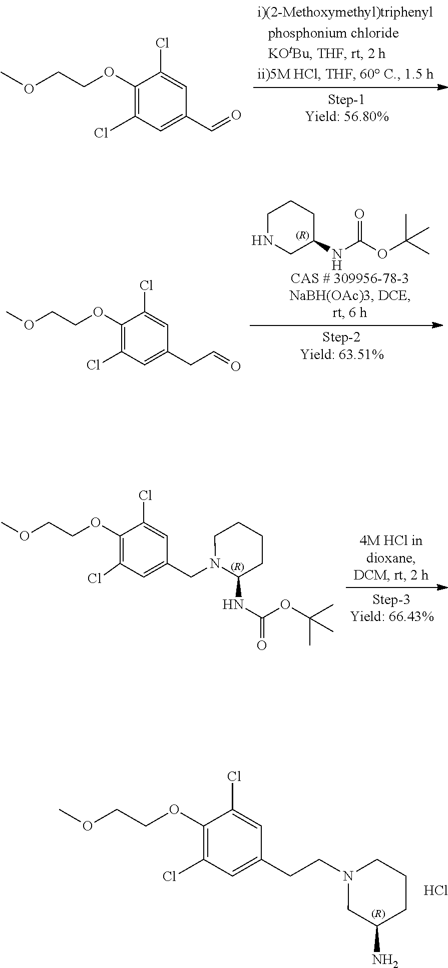

### Step-1: Synthesis of 2-(3,5-dichloro-4-(2-methoxyethoxy)phenyl)acetaldehyde

To a stirred solution of 2-(methoxymethyl)triphenylphosphonium chloride (3.4 g, 10.03 mmol) in THF (20 mL, 20V) at 0° C., 1M potassium tert-butoxide (KTB) in THF (20 mL, 20.07 mmol) was added. After 1 h, 3,5-dichloro-4-(2-methoxyethoxy)benzaldehyde (1.0 g, 0.40 mmol) was added at 0° C. and the reaction mixture was stirred at 0° C. for 10 min. The progress of the reaction was monitored by TLC analysis. After completion of the reaction, the reaction mixture was poured into EtOAc (170 mL) and filtered through Celite®. The filtrate was concentrated under reduced pressure and the crude material purified by flash column chromatography (SiO₂; 100% hexane in EtOAc) to provide (E)-1,3-dichloro-2-(2-methoxyethoxy)-5-(2-methoxyvinyl)benzene (0.7 g, 62.91%) as a yellow viscous liquid. To a stirred solution of (E)-1,3-dichloro-2-(2-methoxyethoxy)-5-(2-methoxyvinyl)benzene (0.7 g, 2.52 mol) in THF (7 mL, 10V) at 0° C., 5M aqueous HCl (3.5 mL, 5V) was added. The reaction was stirred at 60° C. for 1.5 h. The progress of the reaction was monitored by TLC analysis. After completion of the reaction, the reaction mixture was poured into a saturated solution of NaHCO₃ (60 mL) and extracted with EtOAc (3×20 mL). The combined organic fractions were dried over sodium sulfate and concentrated under reduced pressure to provide 2-(3,5-dichloro-4-(2-methoxyethoxy)phenyl)acetaldehyde (0.6 g, 56.80% yield) as a yellow viscous liquid which was used directly in the next step of the synthesis.

### Step-2: Synthesis of tert-butyl (R)-(1-(3,5-dichloro-4-(2-methoxyethoxy)phenethyl)piperidin-3-yl)carbamate

To a stirred solution of 2-(3,5-dichloro-4-(2-methoxyethoxy)phenyl)acetaldehyde (0.3 g, 1.14 mmol) in DCE (3 mL, 10V) at room temperature, tert-butyl (R)-piperidin-3-ylcarbamate (0.27 g, 1.36 mmol) and AcOH (0.015 mL) were added. After 1.5 h, sodium triacetoxyborohydride (0.72 g, 3.42 mmol) was added portion-wise at 0° C. Then the reaction mixture was stirred at room temperature for 4.5 h. The progress of the reaction was monitored by TLC and LCMS analysis. After completion of the reaction, the reaction mixture was poured into a saturated solution of NaHCO₃ (30 mL) and extracted with CH₂Cl₂ (3×20 mL). The combined organic fractions were dried over sodium sulfate and concentrated under reduced pressure. The crude material was purified by column chromatography (SiO₂; 2% MeOH in DCM) to provide tert-butyl (R)-(1-(3,5-dichloro-4-(2-methoxyethoxy)phenethyl)piperidin-3-yl)carbamate (0.32, 63.51% yield) as a yellow viscous liquid.

LCMS [M, M, M+2]: 447.0, 448.9 (RT: 1.422 min, Purity: 83.55%)

### Step-3: Synthesis of (R)-1-(3,5-dichloro-4-(2-methoxyethoxy)phenethyl)piperidin-3-amine hydrochloride (Compound 76)

To a stirred solution of tert-butyl (R)-(1-(3,5-dichloro-4-(2-methoxyethoxy)phenethyl)piperidin-3-yl)carbamate (0.32 g, 0.71 mmol) in CH₂Cl₂ (3.2 mL, 10V) at 0° C., 4M HCl in dioxane (1.6 mL, 5V) was added. The reaction mixture was stirred at room temperature for 2 h. The progress of the reaction was monitored by TLC and LCMS analysis. After completion of the reaction, the reaction mixture was concentrated under reduced pressure and azeotroped with CH₂Cl₂ (3×16 mL). The crude material was triturated using CH₂Cl₂ (3 mL) and diethyl ether (2×20 mL) to provide (R)-1-(3,5-dichloro-4-(2-methoxyethoxy)phenethyl)piperidin-3-amine hydrochloride (0.16 g, 63.43% yield) as an off-white sticky solid.

LCMS [ESI, M, M+2]: 346.9, 348.8 (RT: 1.602 min, Purity: 100%)

HPLC: (RT: 4.817 min, Purity: 100%)

Chiral HPLC: (RT: 6.85 min, Purity: 95.20%)

Instrument Name: Waters SFC Investigator with PDA detector

Sample Name: SLN5-B-679-SLN5-X-1042-036

Chromatographic separation was conducted with Waters SFC Investigator system with PDA detector. The column used was Chiral ICT (250 mm×4.6 mm, 5 μm) and the compounds were eluted with, Mobile Phase A: Liq·CO2, Mobile Phase B: 0.1% Diethyl amine in 2-Propanol with Isocratic method (70:30); with Flow rate=4 ml/min; analysis time 15+F3 min.

¹H NMR (400 MHz, CD₃OD): δ 7.27 (s, 2H), 4.13-4.12 (m, 2H), 3.74-3.68 (m, 3H), 3.55-3.52 (m, 2H), 3.37-3.34 (m, 5H), 2.98-2.94 (m, 4H), 2.14 (d, J=12.4 Hz, 1H), 2.03 (d, J=15.9 Hz, 1H), 1.80-1.70 (m, 1H), 1.61-1.52 (m, 1H).

### Experimental Protocol for Compound 77

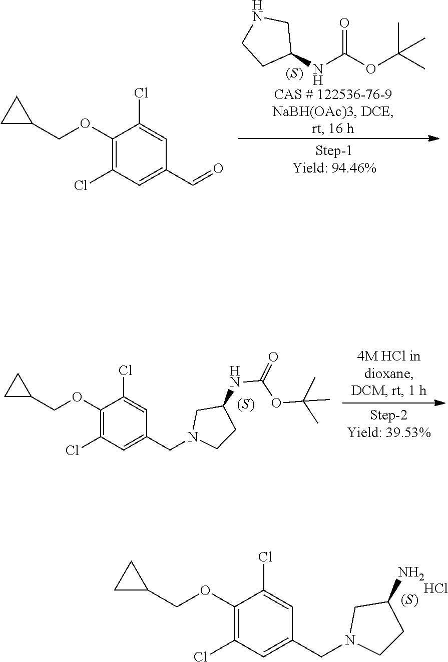

### Step-1: Synthesis of tert-butyl (S)-(1-(3,5-dichloro-4-(cyclopropylmethoxy)benzyl)pyrrolidin-3-yl)carbamate

To a stirred solution of 3,5-dichloro-4-(cyclopropylmethoxy)benzaldehyde (0.25 g, 1.02 mmol) in DCE (5.0 mL) at room temperature, tert-butyl (S)-pyrrolidin-3-ylcarbamate (0.22 g, 1.22 mmol) was added. After 1 h, sodium triacetoxyborohydride (0.64 g, 3.06 mmol) was added at 0° C. and the resulting reaction mixture was stirred at room temperature for 16 h. The progress of the reaction was monitored by TLC analysis. After completion of the reaction, the reaction mixture was poured into water (50 mL) and extracted with CH₂Cl₂ (3×30 mL). The combined organic fractions were dried over sodium sulfate and concentrated under reduced pressure. The crude material was purified by reverse phase column chromatography (74% CH₃CN in water) to provide tert-butyl (S)-(1-(3,5-dichloro-4-(cyclopropylmethoxy)benzyl)pyrrolidin-3-yl)carbamate (0.4 g, 94.46%) as a pale yellow oil.

¹H NMR (400 MHz, CD₃OD): δ 7.37 (s, 2H), 4.07-4.05 (m, 1H), 3.88 (d, J=8.0 Hz, 2H), 3.56 (s, 1H), 2.81-2.77 (m, 1H), 2.68-2.66 (m, 1H), 2.52-2.51 (m, 1H), 2.41-2.38 (m, 1H), 2.23-2.21 (m, 1H), 1.65-1.63 (m, 1H), 1.35 (s, 9H), 1.33-1.31 (m, 1H), 0.63-0.59 (m, 2H), 0.36-0.32 (m, 2H).

### Step-2: Synthesis of (S)-1-(3,5-dichloro-4-(cyclopropylmethoxy)benzyl)pyrrolidin-3-amine hydrochloride (Compound 77)

To a stirred solution of tert-butyl (S)-(1-(3,5-dichloro-4-(cyclopropylmethoxy)benzyl)pyrrolidin-3-yl)carbamate (0.4 g, 0.96 mmol) in CH₂C₁₂ (4 mL) at 0° C., 4M HCl in Dioxane (2.0 mL) was added. The reaction mixture was stirred at room temperature for 1 h. The progress of the reaction was monitored by TLC analysis. After completion of the reaction, the reaction mixture was concentrated under reduced pressure and triturate with diethyl ether. The crude material was purified by reverse phase column chromatography (80:20 CH₃CN:H₂O containing 0.05% aq. HCl) to provide (S)-1-(3,5-dichloro-4-(cyclopropylmethoxy)benzyl)pyrrolidin-3-amine hydrochloride (0.12 g, 39.53% yield) as a white sticky solid.

LCMS [ESI, M, M+2]: 315.2, 317.2 (RT: 3.614 min, Purity: 97.86%),

Chiral HPLC: RT: 6.07 min, Purity: 98.96%

Instrument Name: Waters SFC Investigator with PDA detector

Sample Name: SLN5-B-627-SLN5-X-1026-163-b

Chromatographic separation was conducted with Waters SFC Investigator system with PDA detector. The column used was Chiral ICT (250 mm×4.6 mm, 5 μm) and the compounds were eluted with, Mobile Phase A: Liq·CO2, Mobile Phase B: 0.1% Diethyl amine in 2-Propanol with Isocratic method (50:50); with Flow rate=4 ml/min; analysis time 12 min.

¹H NMR (400 MHz, D₂O): δ 7.47 (s, 2H), 4.33 (s, 2H), 4.13-4.10 (m, 1H), 3.88 (d, J=7.6 Hz, 2H), 3.79 (t, J=12.4 Hz, 1H), 3.49-3.43 (m, 3H), 2.56-2.51 (m, 1H), 2.15-2.10 (m, 1H), 1.30-1.24 (m, 1H), 0.54-0.51 (m, 2H), 0.26-0.24 (m, 2H).

### Experimental Protocol for Compound 78

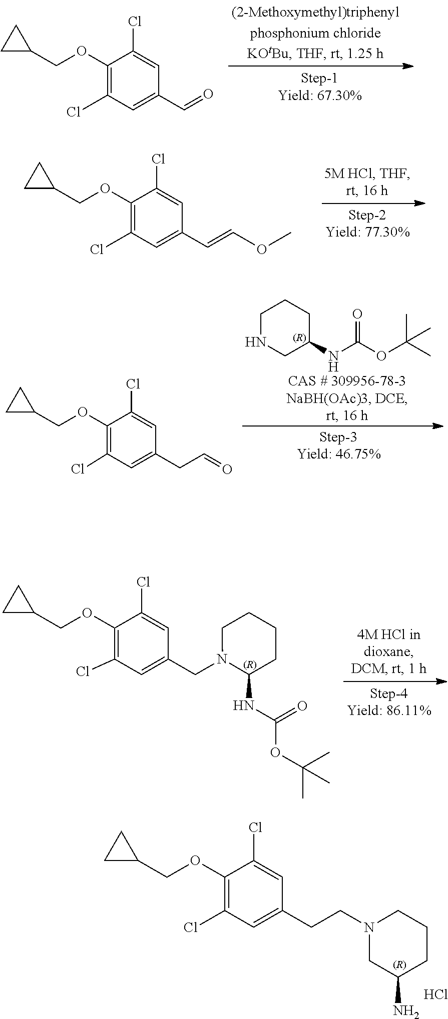

### Step-1: Synthesis of (E)-1,3-dichloro-2-(cyclopropylmethoxy)-5-(2-methoxyvinyl)benzene

To a stirred solution of (2-Methoxymethyl)triphenyl phosphonium chloride (1.39 g, 4.07 mmol) in anhydrous THF (8 mL, 20V) at 0° C., potassium tert-butoxide 1M in THF (8.1 mL, 8.15 mmol) was added. The reaction mixture was stirred at 0° C. for 1 h. Then 3,5-dichloro-4-(cyclopropylmethoxy) benzaldehyde (0.4 g, 1.63 mmol) was added at 0° C. and the reaction mixture was stirred at room temperature for 15 min. The progress of the reaction was monitored by TLC. After completion of the reaction, the reaction mixture was diluted with EtOAc (15 mL) and filtered through Celite®. The filtrate was concentrated under reduced pressure. The crude material was purified by flash column chromatography (SiO₂; 1% EtOAc in hexane) to provide (E)-1,3-dichloro-2-(cyclopropylmethoxy)-5-(2methoxyvinyl)benzene (0.3 g, 67.30 yield %) as a white sticky liquid which was used directly in the next step of the synthesis.

### Step-2: Synthesis of 2-(3,5-dichloro-4-(cyclopropylmethoxy)phenyl)

To a stirred solution of (E)-1,3-dichloro-2-(cyclopropylmethoxy)-5-(2-methoxyvinyl)benzene (0.3 g, 1.09 mmol) in THF (3 mL, 10V) at room temperature, 5M aqueous HCl (1.5 mL, 5V) was added. The reaction mixture was stirred at room temperature for 16 h. The progress of the reaction was monitored by TLC analysis. After completion of the reaction, the reaction mixture was poured into a saturated solution of NaHCO₃ (15 mL) and extracted with EtOAc (2×15 mL). The combined organic fractions were dried over sodium sulfate and concentrated under reduced pressure to provide 2-(3,5-dichloro-4-(cyclopropylmethoxy)phenyl)acetaldehyde (0.22 g, 77.30%) as a white sticky liquid which was used directly in the next step of the synthesis.

### Step-3: Synthesis of tert-butyl (R)-(1-(3,5-dichloro-4-(cyclopropylmethoxy)phenethyl) piperidin-3-yl)carbamate

To a solution of 2-(3,5-dichloro-4-(cyclopropylmethoxy)phenyl)acetaldehyde (0.2 g, 0.7 mmol) in DCE (2 mL, 10V) at room temperature, tert-butyl (R)-piperidin-3-ylcarbamate (0.18 g, 0.92 mmol) was added. After 2 h, NaBH(OAc)₃ (0.48 g, 2.31 mmol) was added at room temperature and the reaction mixture was stirred at room temperature for 16 h. The progress of the reaction was monitored by TLC analysis. After completion of the reaction, the reaction mixture was poured into water (40 mL) and extracted with CH₂Cl₂ (2×80 mL). The combined organic fractions were dried over sodium sulfate and concentrated under reduced pressure. The crude material was purified by reverse phase flash column chromatography (81% CH₃CN in water) to provide tert-butyl (R)-(1-(3,5-dichloro-4(cyclopropylmethoxy)phenethyl)piperidin-3-yl)carbamate (0.16 g, 46.75% yield) as a yellow sticky solid.

LCMS [ESI, M, M+2]: 443.3, 445.5 (RT: 2.127 min, Purity: 91.47%)

### Step-4: Synthesis (R)-1-(3,5-dichloro-4-(cyclopropylmethoxy)phenethyl)piperidin-3-amine hydrochloride (Compound 78)

To a stirred solution of tert-butyl (R)-(1-(3,5-dichloro-4-(cyclopropylmethoxy)phenethyl) piperidin-3-yl)carbamate (0.15 g, 0.33 mmol) in CH₂Cl₂ (1.5 mL, 10V) at 0° C., 4M HCl in Dioxane (0.7 mL, 5V) was added. The reaction mixture was stirred at room temperature for 1 h. The progress of the reaction was monitored by TLC analysis. After completion of the reaction, the reaction mixture was concentrated under reduced pressure and azeotroped with CH₂Cl₂ (3×10 mL). The crude material was purified by trituration using diethyl ether (12 mL): n-Pentane (8 mL) to provide (R)-1-(3,5-dichloro-4-(cyclopropylmethoxy)phenethyl)piperidin-3-amine hydrochloride (0.1 g, 86.11%) as a yellow sticky solid.

LCMS [ESI, M, M+2]: 343.2, 345.2 (RT: 3.795 min, Purity: 95.66%),

HPLC: RT: 4.091 min, Purity: 99.46%

Chiral HPLC: RT: 5.44 min, Purity: 97.06%

Instrument Name: Waters SFC Investigator

Sample Name: SLN5-B-591-SLN5-X-1024-199 a

Chromatographic separation was conducted with Waters SFC Investigator system with 2998 PDA detector. The column used was (Chiral ICT 250×4.6 mm, 5 μm) and the compounds were eluted with, Mobile Phase A: Liq·CO2, Mobile Phase B: 0.1% Diethyl amine in 2-Propanol with Isocratic method (70:30); with Flow rate=4 ml/min; analysis time 12 min.

¹H NMR (400 MHz, D₂O): δ 7.27 (s, 2H), 3.89 (d, J=7.6 Hz, 2H), 3.83 (d, J=7.6 Hz, 1H), 3.56 (bs, 2H), 3.39-3.33 (m, 2H), 2.99-2.90 (m, 4H), 2.15 (d, J=10.8 Hz, 1H), 2.05 (d, J=14.4 Hz, 1H), 1.77-1.74 (m, 1H), 1.63-1.57 (m, 1H), 1.28-1.22 (m, 1H), 0.51 (q, J=12.8, 6.0 Hz, 2H), 0.25 (q, J=10, 4.4 Hz, 2H).

### Experimental Protocol for Compound 79

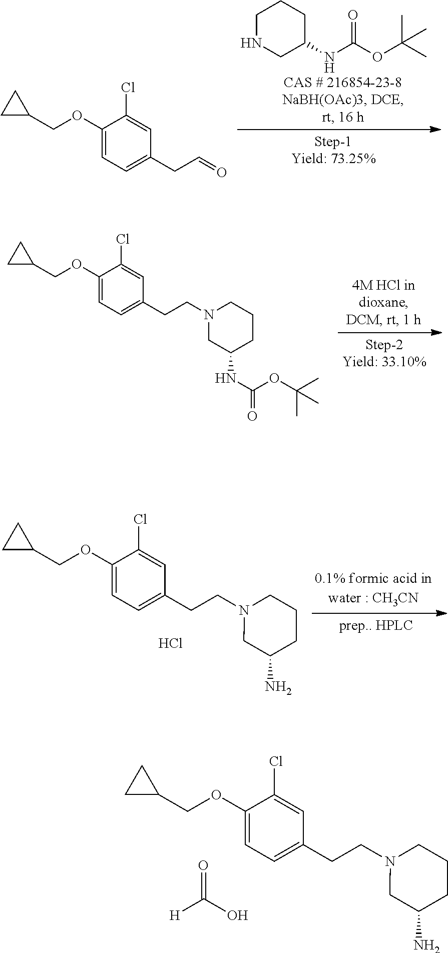

### Step-1: Synthesis of tert-butyl (S)-(1-(3-chloro-4-(cyclopropylmethoxy)phenethyl)piperidin-3-yl)carbamate

To a stirred solution of 2-(3-chloro-4-(cyclopropylmethoxy)phenyl)acetaldehyde (0.3 g, 1.33 mmol) in DCE (5.0 mL) at room temperature, tert-butyl (S)-piperidin-3-ylcarbamate (0.31 g, 1.59 mmol) was added. After 1 h, sodium triacetoxyborohydride (0.84 g, 3.99 mmol) was added at 0° C. and the resulting reaction mixture was stirred at room temperature for 16 h. The progress of the reaction was monitored by TLC analysis. After completion of the reaction, the reaction mixture was poured into water (50 mL) and extracted with CH₂Cl₂ (3×30 mL). The combined organic fractions were dried over sodium sulfate and concentrated under reduced pressure. The crude material was purified by reverse phase column chromatography (70% CH₃CN in water) to provide tert-butyl (S)-(1-(3-chloro-4-(cyclopropylmethoxy)phenethyl)piperidin-3-yl)carbamate (0.4 g, 73.25%) as an off-white liquid.

LCMS [ESI, M, M+2]: 409.1, 411.0 (RT: 1.626 min, Purity: 96.44%),

### Step-2: Synthesis of (S)-1-(3-chloro-4-(cyclopropylmethoxy)phenethyl)piperidin-3-amine formate salt (Compound 79)

To a stirred solution tert-butyl (S)-(1-(3-chloro-4-(cyclopropylmethoxy)phenethyl)piperidin-3-yl)carbamate (0.4 g, 0.97 mmol) in CH₂C₁₂ (5 mL) at 0° C., 4M HCl in Dioxane (2.0 mL) was added. The reaction mixture was stirred at room temperature for 1 h. The progress of the reaction was monitored by TLC analysis. After completion of the reaction, the reaction mixture was concentrated under reduced pressure and triturated with diethyl ether. The crude material was purified by preparative HPLC (0.1% formic acid in water: CH₃CN) to provide (S)-1-(3-chloro-4-(cyclopropylmethoxy)phenethyl)piperidin-3-amine formate salt (0.1 g, 33.10% yield) as an off-white sticky solid.

LCMS [ESI, M, M+2]: 308.9, 310.7 (RT: 1.104 min, Purity: 97.81%),

HPLC: RT: 3.31 min, Purity: 96.39%,

Chiral HPLC: RT: 5.78 min, Purity: 97.30%,

Instrument Name: Waters SFC Investigator with PDA detector

Chromatographic separation was conducted with Waters SFC Investigator system with PDA detector. The column used was Chiral ICT (250 mm×4.6 mm, 5 μm) and the compounds were eluted with, Mobile Phase A: Liq·CO2, Mobile Phase B: 0.1% Diethyl amine in 2-Propanol: acetonitrile (70:30) with Isocratic method (50:50); with Flow rate=4 ml/min; analysis time 12 min.

¹H NMR (400 MHz, D₂O): δ 8.33 (s, 1H), 7.25 (s, 1H), 7.06 (d, J=8.0 Hz, 1H), 6.96 (d, J=8.0 Hz, 1H), 3.81 (d, J=4.0 Hz, 2H), 3.29 (t, J=8.0 Hz, 2H), 3.12 (d, J=12.0 Hz, 1H), 2.88 (d, J=8.0 Hz, 2H), 2.76 (t, J=12.0 Hz, 2H), 2.45-2.42 (m, 2H), 2.0 (d, J=12.0 Hz, 1H), 1.84 (d, J=16.0 Hz, 1H), 1.63-1.53 (m, 1H), 1.42-1.34 (m, 1H), 1.19-1.13 (m, 1H), 0.54-0.50 (m, 2H), 0.25-0.23 (m, 2H).

### Experimental Protocol for Compound 80

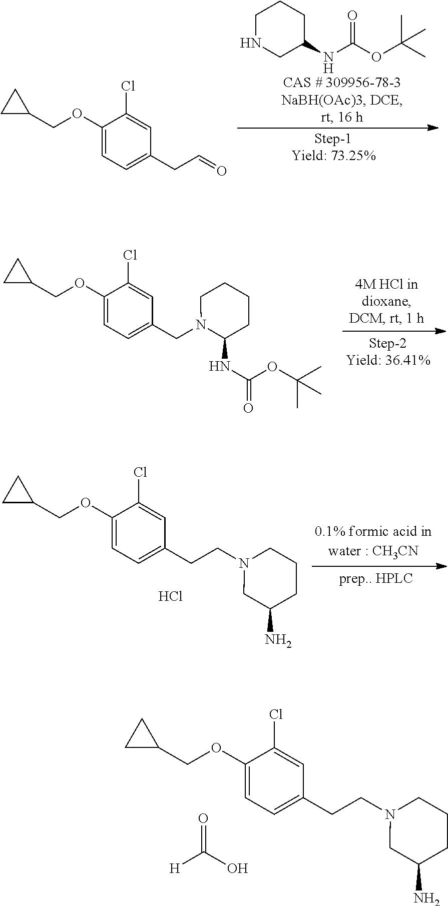

### Step-1: Synthesis of tert-butyl (R)-(1-(3-chloro-4-(cyclopropylmethoxy)phenethyl)piperidin-3-yl)carbamate

To a stirred solution of 2-(3-chloro-4-(cyclopropylmethoxy)phenyl)acetaldehyde (0.3 g, 1.33 mmol) in DCE (5.0 mL) at room temperature, tert-butyl (R)-piperidin-3-ylcarbamate (0.31 g, 1.59 mmol) was added. After 1 h, sodium triacetoxyborohydride (0.84 g, 3.99 mmol) was added at 0° C. and the resulting reaction mixture was stirred at room temperature for 16 h. The progress of the reaction was monitored by TLC analysis. After completion of the reaction, the reaction mixture was poured into water (50 mL) and extracted with CH₂Cl₂ (3×30 mL). The combined organic fraction were dried over sodium sulfate and concentrated under reduced pressure. The crude material was purified by reverse phase column chromatography (70% CH₃CN in water) to provide tert-butyl (R)-(1-(3-chloro-4-(cyclopropylmethoxy)phenethyl)piperidin-3-yl)carbamate (0.4 g, 73.25%) as an off-white liquid.

LCMS [ESI, M+1]: 409.1 (RT: 1.592 min, Purity: 97.83%),

### Step-2: Synthesis of (R)-1-(3-chloro-4-(cyclopropylmethoxy)phenethyl)piperidin-3-amine formate salt (Compound 80)

To a stirred solution tert-butyl (R)-(1-(3-chloro-4-(cyclopropylmethoxy)phenethyl)piperidin-3-yl)carbamate (0.4 g, 0.97 mmol) in CH₂C₁₂ (5 mL) at 0° C., 4M HCl in Dioxane (2.0 mL) was added. The reaction mixture was stirred at room temperature for 1 h. The progress of the reaction was monitored by TLC analysis. After completion of the reaction, the reaction mixture was concentrated under reduced pressure and triturated with diethyl ether. The crude material was purified by preparative HPLC purification (0.1% formic acid in water: CH₃CN) to provide (R)-1-(3-chloro-4-(cyclopropylmethoxy)phenethyl)pyrrolidin-3-amine formate salt (0.11 g, 36.41% yield) as an off-white sticky solid.

LCMS [ESI, M, M+2]: 308.9, 310.6 (RT: 1.103 min, Purity: 97.68%),

HPLC: RT: 3.61 min, Purity: 98.22%,

Chiral HPLC: RT: 5.56 min, Purity: 96.82%

Instrument Name: Waters SFC Investigator with PDA detector

Sample Name: SLN5-B-627-SLN5-X-1014-155-c(R-isomer)

Chromatographic separation was conducted with Waters SFC Investigator system with PDA detector. The column used was Chiral ICT (250 mm×4.6 mm, 5 μm) and the compounds were eluted with, Mobile Phase A: Liq·CO2, Mobile Phase B: 0.1% Diethyl amine in 2-Propanol: acetonitrile (70:30) with Isocratic method (50:50); with Flow rate=4 ml/min; analysis time 12 min.

¹H NMR (400 MHz, D₂O): δ 8.32 (s, 1H), 7.25 (s, 1H), 7.07 (d, J=8.0 Hz, 1H), 6.97 (d, J=8.0 Hz, 1H), 3.82 (d, J=8.0 Hz, 2H), 3.31 (t, J=12.0 Hz, 2H), 3.14 (d, J=8.0 Hz, 1H), 2.91 (d, J=8.0 Hz, 2H), 2.78 (t, J=8.0 Hz, 2H), 2.47 (q, J=20, 12.0 Hz, 2H), 2.01 (d, J=12.0 Hz, 1H), 1.85 (d, J=16.0 Hz, 1H), 1.64-1.54 (m, 1H), 1.44-1.35 (m, 1H), 1.19-1.13 (m, 1H), 0.54-0.50 (m, 2H), 0.25-0.23 (m, 2H).

### Experimental Protocol for Compound 81

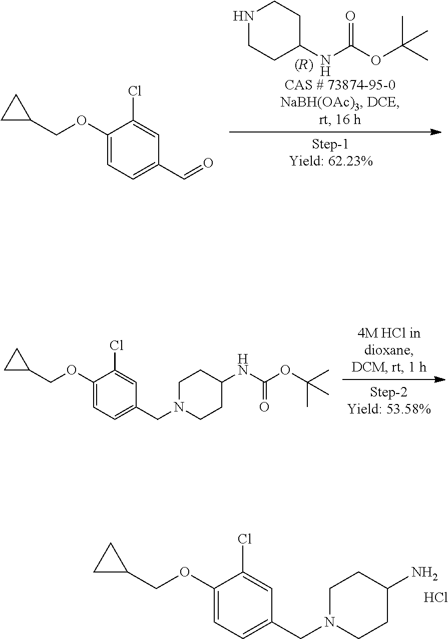

### Step-1: Synthesis of tert-butyl (1-(3-chloro-4-(cyclopropylmethoxy)benzyl)piperidin-4-yl)carbamate

To a stirred solution of 3-chloro-4-(cyclopropylmethoxy)benzaldehyde (0.3 g, 1.42 mmol) in DCE (6 mL) at room temperature, tert-butyl piperidin-4-ylcarbamate (0.34 g, 1.70 mmol) was added. After 1 h, Sodium triacetoxyborohydride (0.9 g, 4.27 mmol) was added at 0° C. and the resulting reaction mixture was stirred at room temperature for 16 h. The progress of the reaction was monitored by TLC analysis. After completion of the reaction, the reaction mixture was poured into water (50 mL) and extracted with CH₂Cl₂ (3×50 mL). The combined organic fractions were dried over sodium sulfate and concentrated under reduced pressure. The crude material was purified by normal phase column chromatography (2% MeOH in CH₂Cl₂) to provide tert-butyl (1-(3-chloro-4-(cyclopropylmethoxy)benzyl)piperidin-4-yl)carbamate (0.35 g, 62.23%) as a pale yellow oil.

LCMS [ESI, M+, M+2]: 395.4, 397.4 (RT: 1.817 min, Purity: 75.98%),

### Step-2: Synthesis of 1-(3-chloro-4-(cyclopropylmethoxy)benzyl)piperidin-4-amine hydrochloride (Compound 81)

To a stirred solution tert-butyl (1-(3-chloro-4-(cyclopropylmethoxy)benzyl)piperidin-4-yl)carbamate (0.35 g, 0.88 mmol) in CH₂Cl₂ (5 mL) at 0° C., 4M HCl in Dioxane (2 mL) was added. The reaction mixture was stirred at room temperature for 1 h. The progress of the reaction was monitored by TLC analysis. After completion of the reaction, the reaction mixture was concentrated under reduced pressure. The crude material was purified by reverse phase column chromatography (7% CH₃CN in water) to provide 1-(3-chloro-4-(cyclopropylmethoxy)benzyl)piperidin-4-amine hydrochloride (0.14 g, 53.58% yield) as a white solid.

LCMS [ESI, M+, M+2]: 294.8, 296.6 (RT: 0.916 min, Purity: 98.57%),

HPLC: RT: 4.638 min, Purity: 95.10%, ¹H NMR (400 MHz, CD₃OD) δ 7.64 (d, J=2.0 Hz, 1H), 7.46 (dd, J=8.0, 2.0 Hz, 1H), 7.16 (d, J=12.0 Hz, 1H), 4.29 (s, 2H), 3.97 (q, J=6.8, 2.4 Hz, 2H), 3.60 (d, J=12.0 Hz, 2H), 3.50-3.45 (m, 1H), 3.15 (t, J=12.0 Hz, 2H), 2.27 (d, J=12.0 Hz, 2H), 2.02 (q, J=24, 12.0 Hz, 2H), 1.35-1.28 (m, 1H), 0.69-0.65 (m, 2H), 0.43-0.41 (m, 2H).

### Experimental Protocol for Compound 82

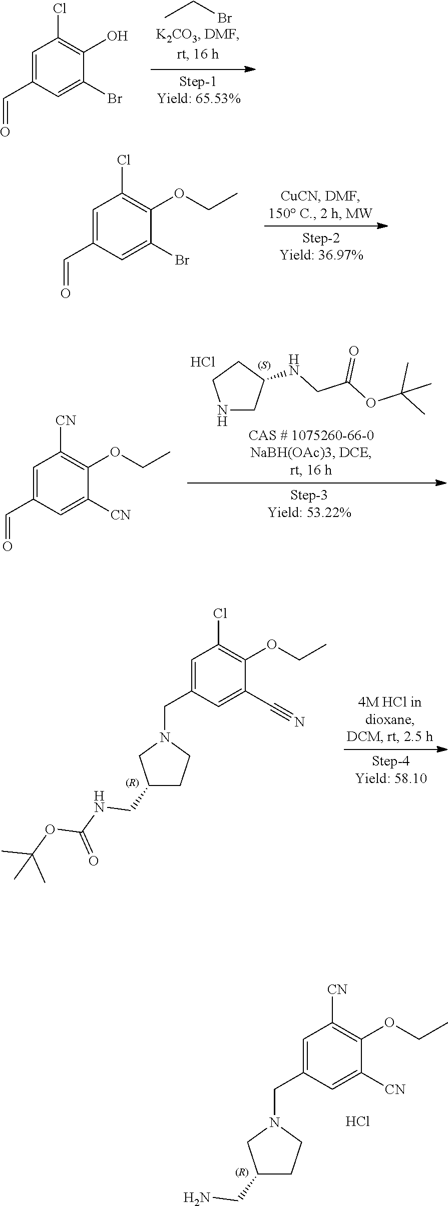

### Step-1: Synthesis of 3-bromo-5-chloro-4-ethoxybenzaldehyde

To a stirred solution of 3-bromo-5-chloro-4-hydroxybenzaldehyde (3.0 g, 12.7 mmol) in DMF (30 mL) at room temperature, K₂CO₃ (5.28 g, 38.2 mmol) was added. After 30 min, bromoethane (2.77 g, 25.4 mmol) was added at room temperature and the reaction mixture was stirred at room temperature for 16 h. The progress of the reaction was monitored by TLC analysis. After completion of the reaction, the reaction mixture was diluted with ice cold water (150 mL) and the solid precipitate was isolated by filtration and concentrated under reduced pressure to provide 3-bromo-5-chloro-4-ethoxybenzaldehyde (2.2 g, 65.53% yield) as a white solid.

¹H NMR (400 MHz, d₆-DMSO): δ 9.90 (s, 1H), 8.15 (d, J=1.6 Hz, 1H), 8.04 (d, J=1.6 Hz, 1H), 4.15-4.13 (m, 2H), 1.41 (t, J=7.2 Hz, 3H).

### Step-2: Synthesis of 3-chloro-2-ethoxy-5-formylbenzonitrile

To a stirred solution of 3-bromo-5-chloro-4-ethoxybenzaldehyde (0.7 g, 2.65 mmol) in DMF (7 mL, 10V) at room temperature, CuCN (0.59 g, 6.64 mmol) was added. The reaction mixture was stirred at 150° C. for 2 h in microwave. The progress of the reaction was monitored by TLC and LCMS analysis. After completion of the reaction, the reaction mixture was concentrated under reduced pressure and the crude material purified by normal phase chromatography (32% EtOAc in Hexane) to provide 3-chloro-2-ethoxy-5-formylbenzonitrile (0.5 g, 36.97% yield) as a white solid. Note that two batches of this reaction were carried out (one batch using 0.7 g of starting aldehyde and one batch using 0.5 g of starting material, with the amount of CuCN adjusted proportionally) and the product yield quoted here is based on the combination of these two batches.

### Synthesis of tert-butyl (R)-((1-(3-chloro-5-cyano-4-ethoxy benzyl) pyrrolidin-3-yl) methyl) carbamate

To a stirred solution of 3-chloro-2-ethoxy-5-formyl benzonitrile (0.3 g, 1.43 mmol) in DCE (4 mL) at room temperature, tert-butyl (S)-(pyrrolidin-3-ylmethyl) carbamate (0.34 g, 1.71 mmol) was added. After 1 h, NaBH (OAc)₃ (0.9 g, 4.29 mmol) was added at 0° C. Then the reaction mixture was stirred at room temperature for 16 h. The progress of the reaction was monitored by TLC analysis. After completion of the reaction, the reaction mixture was diluted with water (100 mL) and extracted with CH₂Cl₂ (2×50 mL). The combined organic fractions were dried over Na₂SO₄ and concentrated under reduced pressure. The crude material was purified by normal phase column chromatography (5% MeOH in CH₂Cl₂) to provide tert-butyl (R)-((1-(3-chloro-5-cyano-4-ethoxybenzyl) pyrrolidin-3-yl) methyl) carbamate (0.3 g, 53.22% yield) as a colorless liquid.

LCMS [ESI, M, M+2]: 394.0, 396.0 (RT: 1.326 min, Purity: 71.34%),

Chiral HPLC Purity: RT: 3.70 min, Purity: 98.29%,

Instrument Name: Waters ACQUITY UPC2

Chromatographic separation was conducted with Waters SFC Investigator system with PDA detector. The column used was CHIRALPAK® IG (250×4.6 mm, 5 ␣m) and the compounds were eluted with Mobile Phase (A): Liquid CO2 Mobile Phase (B): 0.1% Diethylamine in Methanol:Acetonitrile (50:50).

### Step-4: Synthesis of (R)-5-((3-(aminomethyl) pyrrolidin-1-yl) methyl)-3-chloro-2-ethoxybenzonitrile hydrochloride (Compound 82)

To a stirred solution of tert-butyl (R)-((1-(3-chloro-5-cyano-4-ethoxybenzyl)pyrrolidin-3-yl)methyl) carbamate (0.3 g, 0.76 mmol) in CH₂Cl₂ (3.0 mL) at 0° C., 4M HCl in dioxane (1.5 mL, 5V) was added. The reaction mixture was stirred at room temperature for 2.5 h. The progress of the reaction was monitored by TLC analysis. After completion of the reaction, the reaction mixture was concentrated under reduced pressure and the crude material purified by trituration using diethyl ether (2×10 mL) to provide (R)-5-((3-(aminomethyl) pyrrolidin-1-yl) methyl)-3-chloro-2-ethoxybenzonitrile (0.13 g, 58.10% yield) as an off white solid.

LCMS [ESI, M, M+2]: 293.8, 295.7 (RT: 0.986 min, Purity: 96.59%),

HPLC Purity: RT: 4.09 min, Purity: 97.64%,

¹H NMR (400 MHz, CD₃OD): δ 8.06 (d, J=7.6 Hz, 1H), 7.92 (d, J=5.2 Hz, 1H), 4.59-4.41 (m, 2H) 4.35 (q, J=14, 6.8 Hz, 2H), 3.74-3.66 (m, 2H), 3.53-3.47 (m, 1H), 3.31-3.07 (m, 3H), 2.93-2.74 (m, 1H), 2.46-2.31 (m, 1H), 2.04-1.86 (m, 1H), 1.52-1.46 (m, 3H).

### Experimental Protocol for Compound 83

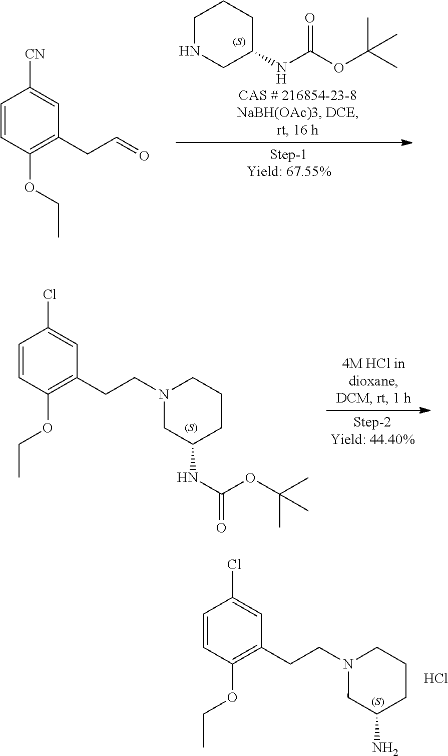

### Step-1: Synthesis of tert-butyl (S)-(1-(5-cyano-2-ethoxy phenethyl) piperidin-3-yl) carbamate

To a stirred solution of 4-ethoxy-3-(2-oxoethyl) benzonitrile (0.15 g, 0.79 mmol) in DCE (1.5 mL, 10V) at room temperature, tert-butyl (S)-piperidin-3-ylcarbamate (0.19 g, 0.95 mmol) was added. For the synthesis of 4-ethoxy-3-(2-oxoethyl) benzonitrile, see steps 1-3 of the synthesis of compound 64 above. The reaction mixture was stirred at room temperature for 1.5 h. Sodium triacetoxyborohydride (0.5 g, 2.38 mmol) was added portion-wise to the reaction mixture at 0° C. Then the reaction mixture was allowed to stirred at room temperature for 16 h. The progress of the reaction was monitored by TLC and LCMS analysis. After completion of the reaction, the reaction mixture was poured into a saturated solution of NaHCO₃ (40 mL) and extracted with CH₂Cl₂ (3×40 mL). The combined organic fractions were dried over anhydrous sodium sulphate and concentrated under reduced pressure. The crude material was purified by normal phase column chromatography (60% MeOH in CH₂Cl₂ with 0.1% aq. Ammonia) to provide tert-butyl (S)-(1-(5-cyano-2-ethoxy phenethyl) piperidin-3-yl) carbamate (0.2 g, 67.55% yield) as a yellow viscous liquid.

LCMS [M+1]: 374.2 (RT: 1.410 min, Purity: 85.99%)

### Step-2: Synthesis of (S)-3-(2-(3-aminopiperidin-1-yl)ethyl)-4-ethoxybenzonitrile hydrochloride (Compound 83)

To a stirred solution of tert-butyl (S)-(1-(5-cyano-2-ethoxy phenethyl) piperidin-3-yl) carbamate (0.2 g, 0.53 mmol) in CH₂Cl₂ (2.0 mL, 10V) at 0° C., 4M HCl in dioxane (0.6 mL, 3V) was added. The reaction mixture was stirred at room temperature for 1 h. The progress of the reaction was monitored by TLC and LCMS analysis. After completion of the reaction, the reaction mixture was concentrated under reduced pressure and the crude material purified by reverse phase column chromatography (55% CH₃CN in H₂O) to provide (S)-3-(2-(3-aminopiperidin-1-yl) ethyl)-4-ethoxybenzonitrile hydrochloride (0.065 g, Yield: 44.40%) as a brown sticky solid.

LCMS [ESI, M+1]: 274.3 (RT: 1.374 min, Purity: 96.62%)

HPLC Purity: RT: 5.59 min, Purity: 99.26%

Chiral HPLC: RT: 1.813 min, Purity: 95.03%

Instrument Name: Waters ACQUITY UPC2

Chromatographic separation was conducted with Waters ACQUITY UPC2 system with 2998 PDA detector. The column used was CHIRALPAK® IG-3 (100×3 mm, 3 ␣m) and the compounds were eluted with Mobile Phase A: Liq·CO2, Mobile Phase B: 0.1% Methanolic ammonia in Methanol:Acetonitrile (50:50) with Gradient method; with Flow rate=2 ml/min; analysis time 4 min.

¹H NMR (400 MHz, D₂O): δ 7.51 (dd, J=8.6, 2.1 Hz, 1H), 7.45 (d, J=2.1 Hz, 1H), 6.96 (d, J=8.6 Hz, 1H), 4.05 (q, J=7.0 Hz, 2H), 2.90 (d, J=10.8 Hz, 1H), 2.74-2.69 (m, 4H), 2.46 (q, J=9.8, 5.8 Hz, 2H), 1.94 (t, J=10.5 Hz, 1H), 1.77 (dd, J=13.1, 7.8 Hz, 2H), 1.65-1.58 (m, 1H), 1.41 (dd, J=24.1, 10.5 Hz, 1H), 1.30 (t, J=7.0 Hz, 3H), 0.92-1.0 (m, 1H)

### Experimental Protocol for Compound 84

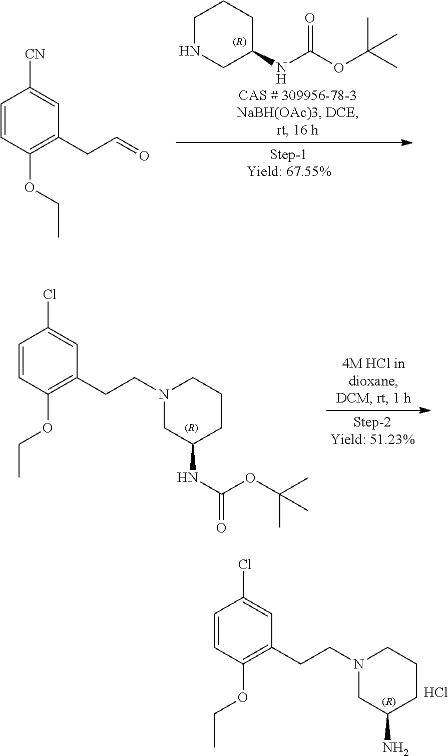

### Step-1: Synthesis of tert-butyl (R)-(1-(5-cyano-2-ethoxyphenethyl) piperidin-3-yl) carbamate

To a stirred solution of 4-ethoxy-3-(2-oxoethyl) benzonitrile (0.15 g, 0.79 mmol) in DCE (1.5 mL, 10V) at room temperature, tert-butyl (R)-piperidin-3-yl carbamate (0.19 g, 0.95 mmol) was added. For the synthesis of 4-ethoxy-3-(2-oxoethyl) benzonitrile, see steps 1-3 of the synthesis of compound 64 above. The reaction mixture was stirred at room temperature for 1.5 h. Sodium triacetoxyborohydride (0.5 g, 2.38 mmol) was added portion-wise to the reaction mixture at 0° C. The reaction mixture was stirred at room temperature for 16 h. The progress of the reaction was monitored by TLC and LCMS analysis. After completion of the reaction, the reaction mixture was poured into a sat. solution of NaHCO₃ (40 mL) and extracted with CH₂Cl₂ (3×30 mL). The combined organic fractions were dried over anhydrous sodium sulphate and concentrated under reduced pressure. The crude material was purified by normal phase column chromatography (5% MeOH in CH₂Cl₂) to provide tert-butyl (R)-(1-(5-cyano-2-ethoxy phenethyl) piperidin-3-yl) carbamate (0.2 g, 67.55% yield) as a yellow viscous liquid.

LCMS [M+1]: 374.1 (RT: 1.408 min, Purity: 94.30%)

### Step-2: Synthesis of (R)-3-(2-(3-aminopiperidin-1-yl) ethyl)-4-ethoxy benzonitrile hydrochloride (Compound 84)

To a stirred solution of tert-butyl (R)-(1-(5-cyano-2-ethoxy phenethyl) piperidin-3-yl) carbamate (0.2 g, 0.53 mmol) in CH₂Cl₂ (2.0 mL, 10V) at 0° C., 4M HCl in dioxane (0.6 mL, 3V) was added. The reaction mixture was stirred at room temperature for 1 h. The progress of the reaction was monitored by TLC and LCMS analysis. After completion of the reaction, the reaction mixture was concentrated under reduced pressure. The crude material was purified by reverse phase column chromatography (60% CH₃CN in H₂O) to provide (R)-3-(2-(3-aminopiperidin-1-yl) ethyl)-4-ethoxy benzonitrile hydrochloride (0.075 g, 51.23%) as a brown sticky solid.

LCMS [ESI, M+1]: 274.3 (RT: 1.480 min, Purity: 100%)

HPLC Purity: RT: 5.62 min, Purity: 99.46%

Chiral HPLC: RT: 1.878 min, Purity: 98.96%

Instrument Name: Waters ACQUITY UPC2

Chromatographic separation was conducted with Waters ACQUITY UPC2 system with 2998 PDA detector. The column used was CHIRALPAK® IG-3 (100×3 mm, 3 ␣m) and the compounds were eluted with, Mobile Phase A: Liq·CO2, Mobile Phase B: 0.1% Methanolic ammonia in Methanol:Acetonitrile (50:50) with Gradient method; with Flow rate=2 ml/min; analysis time 4 min.

¹H NMR (400 MHz, D₂O): δ 7.51 (dd, J=8.8, 2.0 Hz, 1H), 7.43 (d, J=2.0 Hz, 1H), 6.95 (d, J=8.8 Hz, 1H), 4.04 (q, J=6.8, 14 Hz, 2H), 2.89 (d, J=10.4 Hz, 1H), 2.79 (d, J=11.2 Hz, 1H), 2.72-2.67 (m, 3H), 2.45 (q, J=5.6, 9.6 Hz, 2H), 1.91 (t, 1H), 1.78-1.71 (m, 2H), 1.63-1.59 (m, 1H), 1.45-1.35 (m, 1H), 1.29 (t, 3H), 1.27-0.87 (m, 1H).

### Experimental Protocol for Compound 85

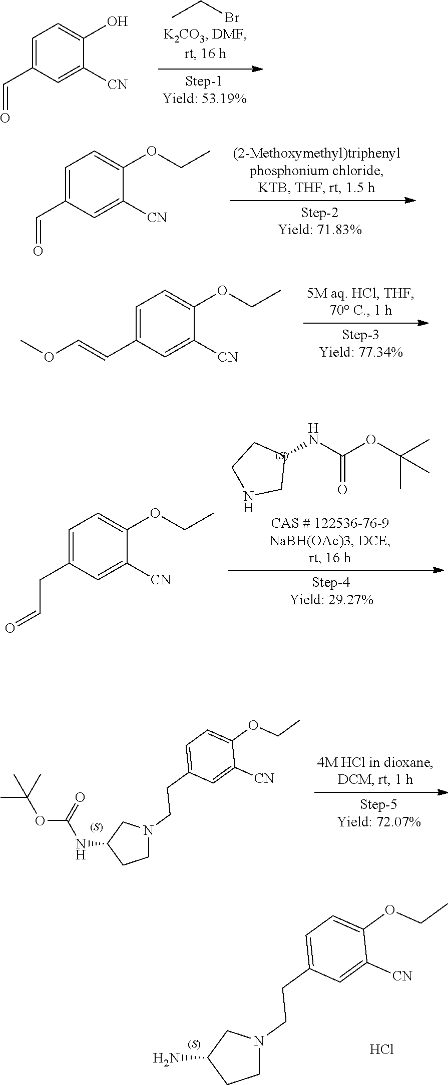

### Step-1: Synthesis of 2-ethoxy-5-formylbenzonitrile

To a stirred solution of 5-formyl-2-hydroxybenzonitrile (3.0 g, 20.39 mmol) in DMF (30 mL) at 0° C., K₂CO₃ (8.44 g, 61.1 mmol) was added. After 30 min, ethyl bromide (3.33 g, 30.5 mmol) was added at 0° C. and the reaction mixture stirred at room temperature for 16 h. The progress of the reaction was monitored by TLC analysis. After completion of the reaction, the reaction mixture was poured into ice-cold water (150 mL). The solid precipitate was isolated by filtration and dried under reduced pressure to provide 2-ethoxy-5-formylbenzonitrile (1.9 g, 53.19%) as an orange solid.

¹H NMR (400 MHz, d₆-DMSO) δ 9.89 (s, 1H), 8.31 (d, J=4.0 Hz, 1H), 8.17 (dd, J=8.0, 2.0 Hz, 1H), 7.45 (d, J=8.0 Hz, 1H), 4.33 (q, J=8.0, 4.0 Hz, 2H), 1.40 (t, J=8.0 Hz, 3H).

### Step-2: Synthesis of (E)-2-ethoxy-5-(2-methoxyvinyl)benzonitrile

To a stirred solution of 2-(Methoxymethyl)triphenyl phosphonium chloride (14.67 g, 42.8 mmol) in THF (120 mL) at 0° C., KTB (1M in THF) (85.5 mL, 85.5 mmol) was added. After 1 h, 2-ethoxy-5-formylbenzonitrile (3.0 g, 17.1 mmol) was added at 0° C. and the resulting reaction mixture stirred at 0° C. for 15 min. The progress of the reaction was monitored by TLC analysis. After completion of the reaction, the reaction mixture was diluted with EtOAc (100 mL) and filtered through Celite®. The filtrate was dried over Na₂SO₄ and concentrated under reduced pressure. The crude material was purified by normal phase column chromatography (10% EtOAc in Hexane) to provide (E)-2-ethoxy-5-(2-methoxyvinyl)benzonitrile (2.5 g, 71.83%) as a pale yellow oil which was used directly in the next step of the synthesis.

### Step-3: Synthesis of 2-ethoxy-5-(2-oxoethyl)benzonitrile

To a stirred solution of (E)-2-ethoxy-5-(2-methoxyvinyl)benzonitrile (2.5 g, 12.3 mmol) in THF (25.0 mL) at room temperature, 5M aq. HCl (12.5 mL, 5.0V) was added. The reaction mixture was stirred at 70° C. for 1 h. The progress of the reaction was monitored by TLC analysis. After completion of the reaction, the reaction mixture was poured into NaHCO₃ solution (50 mL) and extracted with EtOAc (3×50 mL). The combined organic fractions were dried over Na₂SO₄, and concentrated under reduced pressure to provide 2-ethoxy-5-(2-oxoethyl)benzonitrile (1.8 g, 77.34%) as an off white liquid. Product was confirmed by TLC analysis using, 0.4-DNP stain and used directly in the next step.

### Step-4: Synthesis of tert-butyl (S)-(1-(3-cyano-4-ethoxy phenethyl) pyrrolidin-3-yl) carbamate

To a stirred solution of 2-ethoxy-5-(2-oxoethyl) benzonitrile (0.45 g, 23.7 mmol) in DCE (5.0 mL) at room temperature, tert-butyl (S)-pyrrolidin-3-yl carbamate (0.53 g, 2.85 mmol) was added. After 1 h, sodium triacetoxyborohydride (1.5 g, 7.11 mmol) was added at 0° C. and the resulting reaction mixture stirred at room temperature for 16 h. The progress of the reaction was monitored by TLC analysis. After completion of the reaction, the reaction mixture was poured into water (30 mL) and extracted with CH₂Cl₂ (3×30 mL). The combined organic fractions were dried over Na₂SO₄ and concentrated under reduced pressure. The crude material was purified by normal phase column chromatography (4% MeOH in CH₂Cl₂) to provide tert-butyl (S)-(1-(3-cyano-4-ethoxyphenethyl) pyrrolidin-3-yl) carbamate (0.25 g, 29.27%) as a yellow oil.

LCMS [ESI, M+1]: 360.3 (RT: 1.768 min, Purity: 92.08%),

### Step-5: Synthesis of (S)-5-(2-(3-aminopyrrolidin-1-yl) ethyl)-2-ethoxybenzonitrile hydrochloride (Compound 85)

To a stirred solution tert-butyl (S)-(1-(3-cyano-4-ethoxy phenethyl) pyrrolidin-3-yl) carbamate (0.25 g, 0.69 mmol) in CH₂Cl₂ (5.0 mL) at 0° C., 4M HCl in dioxane (2.0 mL) was added. The reaction mixture was stirred at room temperature for 1 h. The progress of the reaction was monitored by TLC analysis. After completion of the reaction, the reaction mixture was concentrated under reduced pressure and the crude material was purified by trituration using diethyl ether to provide (S)-5-(2-(3-aminopyrrolidin-1-yl) ethyl)-2-ethoxybenzonitrile hydrochloride (0.13 g, 63.41% yield) as a brown sticky solid.

LCMS [ESI, M+1]: 260.2 (RT: 1.043 min, Purity: 100%),

HPLC Purity: RT: 3.454 min, Purity: 96.14%,

Chiral HPLC Purity: RT: 6.704 min, Purity: 99.06%,

Instrument Name: Shimadzu LC-20 AD

Chromatographic separation was conducted with Shimadzu LC-20 AD system with DAD detector. The column used was CHIRALPAK® IG (250×4.6 mm, 5 ␣m) and the compounds were eluted with, Mobile Phase A: Methanol with an Isocratic Method (100% A); with Flow rate=1 ml/min; analysis time 12 min.

¹H NMR (400 MHz, CD₃OD): δ 7.63-7.60 (m, 2H), 7.16 (d, J=8.4 Hz, 1H), 4.20 (q, J=14, 7.2 Hz, 3H), 4.13 (s, 1H), 3.94 (t, J=8.0 Hz, 1H), 3.84 (d, J=4.0 Hz, 1H), 3.79-3.53 (m, 3H), 3.11 (brs, 2H), 2.71-2.55 (m, 1H), 2.32-2.10 (m, 1H), 1.46 (t, J=8.0 Hz, 3H).

### Experimental Protocol for Compound 86

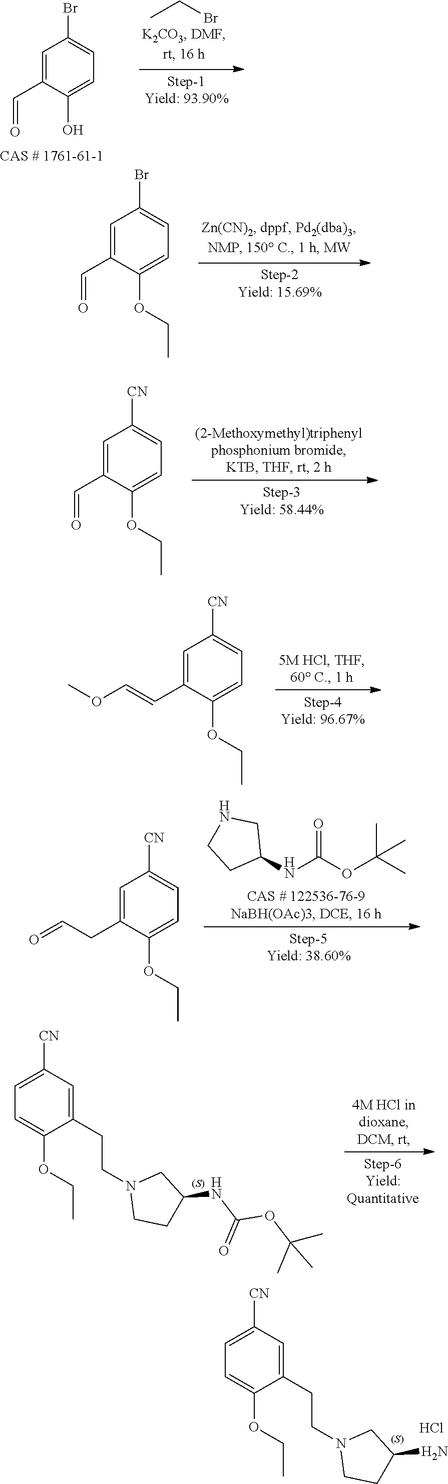

### Step-1: Synthesis of 5-bromo-2-ethoxybenzaldehyde

To a stirred solution of 5-bromo-2-hydroxybenzaldehyde (5.0 g, 24.87 mmol) in DMF (50 mL, 10V) at room temperature, K₂CO₃ (6.87 g, 49.74 mmol) was added. After 30 min, bromoethane (3.25 g, 29.84 mmol) was added at room temperature and the reaction was stirred at room temperature for 16 h. The progress of the reaction was monitored by TLC analysis. After completion of the reaction, ice was added into the reaction mixture and a solid precipitate was formed. The solid material was isolated by filtration and dried under reduced pressure to provide 5-bromo-2-ethoxybenzaldehyde (5.35 g, 93.90%) as a white solid.

¹H NMR (400 MHz, d₆-DMSO): δ 10.30 (s, 1H), 7.81 (dd, J=8.9, 2.7 Hz, 1H), 7.74 (d, J=2.7 Hz, 1H), 7.23 (d, J=8.9 Hz, 1H), 4.20 (q, J=14, 6.8 Hz, 2H), 1.39 (t, J=7.0 Hz, 3H).

### Step-2: Synthesis of 4-ethoxy-3-formylbenzonitrile

To a stirred solution of 5-bromo-2-ethoxybenzaldehyde (0.5 g, 2.18 mmol) in N-Methyl-2-pyrrolidone (NMP; 5 mL, 10V) at room temperature, Zn(CN)₂ (0.51 g, 4.36 mmol) and 1,1′-Bis(diphenylphosphino)ferrocene (0.12 g, 0.21 mmol) were added and the flask was purged with N₂ gas for 15 min. Then, tris(dibenzylideneacetone) dipalladium (0) (0.2 g, 0.21 mmol) was added and the flask again purged with N₂ gas for 10 min. The resulting reaction mixture was stirred at 150° C. for 1 h under microwave irradiation. The progress of the reaction was monitored by TLC analysis. After completion of the reaction, the reaction mixture was poured into water (100 mL), filtered and extracted with EtOAc (3×50 mL). The combined organic fractions were dried over Na₂SO₄ and concentrated under reduced pressure. The crude material was purified by column chromatography (SiO₂; 8% EtOAc in hexane) to provide 4-ethoxy-3-formylbenzonitrile (0.36 g, 15.69%) as a brown solid. Note that six batches of this reaction were carried out all on the same scale and the product yield quoted here is based on the combination of these six batches.

¹H NMR (400 MHz, d₆-DMSO): δ 10.32 (s, 1H), 8.10 (dd, J=8.7, 2.3 Hz, 1H), 8.06 (d, J=2.2 Hz, 1H), 7.43 (d, J=8.8 Hz, 1H), 4.31 (q, J=6.7 Hz, 2H), 1.42 (q, J=7.0 Hz, 3H).

### Step-3: Synthesis of (E)-4-ethoxy-3-(2-methoxyvinyl)benzonitrile

To a stirred solution of (2-Methoxymethyl)triphenyl phosphonium Chloride (1.44 g, 4.20 mmol) in THF (11.8 mL, 40V) at 0° C., KOtBu (KTB; 8.41 mL) was added. The reaction was stirred for 0° C. for 1 h. Than 4-ethoxy-3-formylbenzonitrile (0.29 g, 1.68 mmol) was added at 0° C. and the reaction mixture was stirred at 0° C. for 10 min. The progress of the reaction was monitored by TLC analysis. After completion of the reaction, the reaction mixture was poured into EtOAc (80 mL) and filtered through Celite®. The filtrate was concentrated under reduced pressure and the crude material was purified by column chromatography (SiO₂; % EtOAc in hexane) to provide (E)-4-ethoxy-3-(2-methoxyvinyl)benzonitrile (0.2 g, 58.44%) as a yellow viscous liquid. The isolated material was used directly in the next step of the synthesis.

### Step-4: Synthesis of 4-ethoxy-3-(2-oxoethyl) benzonitrile

To a stirred solution of (E)-4-ethoxy-3-(2-methoxyvinyl) benzonitrile (0.2 g, 0.98 mmol) in THF (2 mL, 10V) at 0° C., 5M HCl (1 mL, 5V) was added. The reaction was stirred for 60° C. for 1 h. The progress of the reaction was monitored by TLC. After completion of the reaction, the reaction mixture was poured into a sat. NaHCO₃ solution (90 mL) and extracted with EtOAc (2×15 mL). The combined organic fractions were dried over Na₂SO₄ and concentrated under reduced pressure to provide 4-ethoxy-3-(2-oxoethyl)benzonitrile (0.18 g, 96.67% yield) as a yellow viscous liquid. The isolated material was used directly in the next step of the synthesis.

### Step-5: Synthesis of tert-butyl (S)-(1-(5-cyano-2-ethoxyphenethyl) pyrrolidin-3-yl) carbamate

To a stirred solution of 4-ethoxy-3-(2-oxoethyl) benzonitrile (0.3 g, 1.58 mmol) in DCE (3 mL, 10V) at room temperature, tert-butyl (S)-pyrrolidin-3-yl carbamate hydrochloride (0.35 g, 1.90 mmol) was added. The reaction mixture was stirred at room temperature for 1.5 h. Then, sodium triacetoxyborohydride (Ig, 4.74 mmol) was added portion-wise to the reaction mixture at 0° C. Then reaction mixture was allowed to stir at room temperature for 16 h. The progress of the reaction was monitored by TLC and LCMS analysis. After completion of the reaction, the reaction mixture was poured into a sat. solution of NaHCO₃ (40 mL) and extracted with CH₂Cl₂ (3×20 mL). The combined organic fractions were dried over anhydrous sodium sulphate and concentrated under reduced pressure. The crude material was purified by column chromatography (SiO₂; 2% MeOH in CH₂Cl₂) to provide tert-butyl (S)-(1-(5-cyano-2-ethoxyphenethyl) pyrrolidin-3-yl) carbamate (0.22, 38.60% yield) as yellow viscous liquid.

LCMS [M+1]: 360.1 (RT: 1.866 min, Purity: 98.78%).

### Step-6: Synthesis of (S)-3-(2-(3-aminopyrrolidin-1-yl) ethyl)-4-ethoxy benzonitrile hydrochloride (Compound 86)

To a stirred solution of tert-butyl (S)-(1-(5-cyano-2-ethoxyphenethyl)pyrrolidin-3-yl)carbamate (0.2 g, 0.55 mmol) in CH₂Cl₂ (2.0 mL, 10V) at 0° C., 4M HCl in dioxane (0.6 mL, 3V) was added. The reaction mixture was stirred at room temperature for 1 h. The progress of the reaction was monitored by TLC and LCMS analysis. After completion of the reaction, the reaction mixture was concentrated under reduced pressure and the crude material triturated using diethyl ether (3×40 mL) and pentane (2×20 mL) to provide (S)-3-(2-(3-aminopyrrolidin-1-yl) ethyl)-4-ethoxybenzonitrile hydrochloride (0.16 g, Quantitative yield) as a brown sticky solid.

LCMS [ESI, M+1]: 260.3 (RT: 1.379 min, Purity: 100%)

HPLC Purity: RT: 5.92 min, Purity: 98.51%

Chiral HPLC: RT: 2.392 min, Purity: 98.18%)

Instrument Name: Waters ACQUITY UPC2

Chromatographic separation was conducted with Waters ACQUITY UPC2 system with 2998 PDA detector. The column used was CHIRALPAK® IC-3 (100×3 mm, 3 ␣m) and the compounds were eluted with, Mobile Phase A: Liq·CO2, Mobile Phase B: 0.1% Methanolic ammonia in Methanol:Acetonitrile (50:50) with Gradient method; with Flow rate=2 ml/min; analysis time 5 min.

¹H NMR (400 MHz, D₂O): δ 7.63 (dd, J=8.8, 2.4 Hz, 1H), 7.53 (d, J=2.0 Hz, 1H), 7.02 (d, J=6.9 Hz, 1H), 4.14 (q, J=7.2 Hz, 3H), 3.46 (t, J=7.2 Hz, 7H), 3.01 (t, J=8.0 Hz, 2H), 2.54 (s, 2H), 2.13 (s, 1H), 1.34 (q, J=6.8 Hz, 3H).

### Experimental Protocol for Compound 87

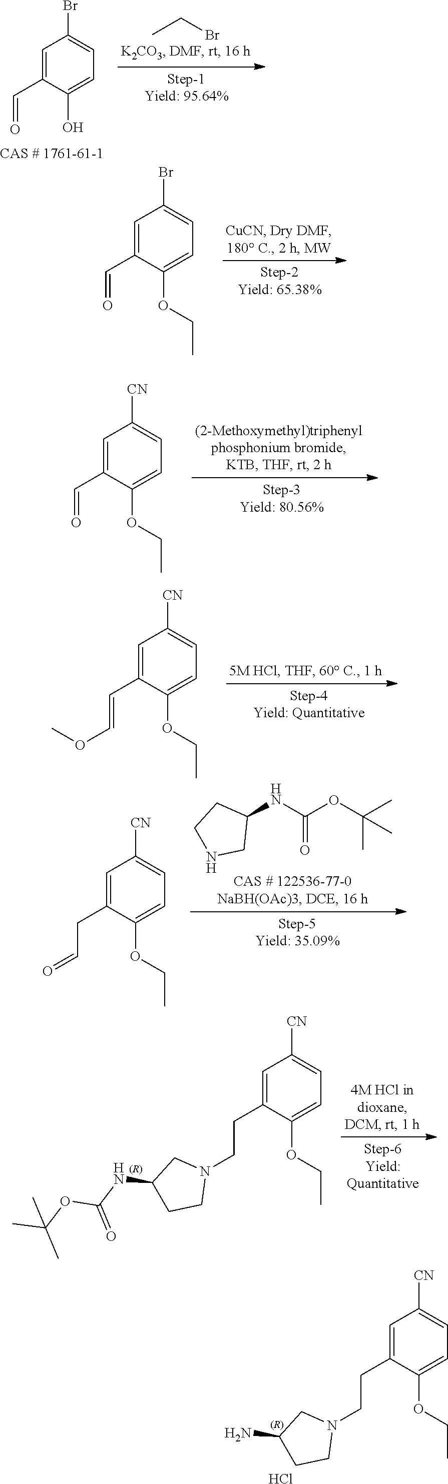

### Step-1: Synthesis of 5-bromo-2-ethoxybenzaldehyde

To a stirred solution of 5-bromo-2-hydroxy benzaldehyde (5 g, 24.87 mmol) in DMF (50 mL, 10V) at room temperature, K₂CO₃ (6.87 g, 49.74 mmol) was added. The reaction was stirred for 30 min at room temperature. Bromoethane (4.05 g, 37.30 mmol) was added at room temperature. The reaction mixture was stirred for 16 h at room temperature. The progress of the reaction was monitored by TLC analysis. After completion of the reaction, ice was added to reaction mixture to form a precipitate. The precipitate was isolated by filtration to provide 5-bromo-2-ethoxybenzaldehyde (5.45 g, 95.64%) as a white solid.

¹H NMR (400 MHz, d₆-DMSO): δ 10.29 (s, 1H), 7.81 (dd, J=8.9, 2.7 Hz, 1H), 7.74 (d, J=2.7 Hz, 1H), 7.23 (d, J=8.9 Hz, 1H), 4.20 (q, J=7.0 Hz, 2H), 1.39 (t, J=7.0 Hz, 3H).

### Step-2: Synthesis of 4-ethoxy-3-formylbenzonitrile

To a stirred mixture of 5-bromo-2-ethoxy benzaldehyde (0.5 g, 2.18 mmol) in DMF (10 mL, 20V) at room temperature, CuCN (0.58 g, 6.55 mmol) was added. The reaction mixture was purged with N₂ gas for 15 min. The reaction mixture was stirred at 180° C. for 2 h under microwave irradiation. The progress of the reaction was monitored by TLC analysis. After completion of the reaction, ice was added to the reaction mixture to form a precipitate which was isolated by filtration. The precipitate was dissolved in CH₂Cl₂ and the mixture filtered. The filtrate was dried over Na₂SO₄ and concentrated under reduced pressure. The crude material was purified by column chromatography (SiO₂; 10% EtOAc in Hexane) to provide 4-ethoxy-3-formylbenzonitrile (1 g, 65.38% yield) as a light yellow solid. Note that four batches of this reaction were carried out all on the same scale and the product yield quoted here is based on the combination of these four batches.

¹H NMR (400 MHz, d₆-DMSO): δ 10.32 (s, 1H), 8.10 (dd, J=8.8, 2.4 Hz, 1H), 8.06 (d, J=2.2 Hz, 1H), 7.43 (d, J=8.8 Hz, 1H), 4.31 (q, J=6.7 Hz, 2H), 1.42 (q, J=7.0 Hz, 3H).

### Step-3: Synthesis of (E)-4-ethoxy-3-(2-methoxyvinyl) benzonitrile

To a stirred solution of (2-methoxymethyl) triphenyl phosphonium chloride (5.2 g, 15.2 mol) in anhydrous THF (40 mL, 40V) at 0° C., KOtBu (1M in THF; 30.5 mL, 5 eq) was added. The reaction was stirred for 0° C. for 1 h. After that 4-ethoxy-3-formyl benzonitrile (lg, 6.10 mol) was added at 0° C. and the reaction mixture was stirred at 0° C. for 10 min. The progress of the reaction was monitored by TLC analysis. After completion of the reaction, the reaction mixture was poured into EtOAc (250 mL) and filtered through Celite®. The filtrate was concentrated under reduced pressure and the crude material was purified by column chromatography (neutral SiO₂; 7% EtOAc in Hexane) to provide (E 4-ethoxy-3-(2-methoxyvinyl) benzonitrile (12, 80.56%) as a white viscous liquid which was immediately used in the next step of the synthesis.

### Step-4: Synthesis of 4-ethoxy-3-(2-oxoethyl) benzonitrile

To a stirred solution of (E)-4-ethoxy-3-(2-methoxyvinyl) benzonitrile (0.5 g, 2.40 mol) in THF (5 mL, 10V) at 0° C., 5M HCl (2.5 mL, 5V) was added. The reaction was stirred for 60° C. for 1 h. The progress of the reaction was monitored by TLC. After completion of the reaction, the reaction mixture was poured into a sat. solution of NaHCO₃ (70 mL) and extracted with EtOAc (3×30 mL). The combined organic fractions were dried over anhydrous sodium sulphate and concentrated under reduced pressure to provide 4-ethoxy-3-(2-oxoethyl) benzonitrile (0.6 g, Quantitative yield) as a yellow viscous liquid which was immediately used in the next step of the synthesis

### Step-5: Synthesis of tert-butyl (R)-(1-(5-cyano-2-ethoxy phenethyl) pyrrolidin-3-yl) carbamate

To a stirred solution of 4-ethoxy-3-(2-oxoethyl) benzonitrile (0.3 g, 1.58 mmol) in DCE (3 mL, 10V) at room temperature, tert-butyl (R)-pyrrolidin-3-yl carbamate hydrochloride (0.35 g, 1.90 mmol) was added. The reaction mixture was stirred at room temperature for 1.5 h. Then, sodium triacetoxyborohydride (lg, 4.74 mmol) was added portion-wise to the reaction mixture at 0° C. The reaction mixture was allowed to stir at room temperature for 16 h. The progress of the reaction was monitored by TLC and LCMS analysis. After completion of the reaction, the reaction mixture was poured into a sat. solution of NaHCO₃ (40 mL) and extracted with CH₂Cl₂ (3×30 mL). The combined organic fractions were dried over anhydrous sodium sulphate and concentrated under reduced pressure. The crude material was purified by column chromatography (SiO₂; 3% MeOH in CH₂Cl₂) to provide tert-butyl (R)-(1-(5-cyano-2-ethoxy phenethyl) pyrrolidin-3-yl) carbamate (0.2 g, 35.09% yield) as a yellow viscous liquid.

LCMS [M+1]: 360 (RT: 1.874 min, Purity: 100%)

### Step-6: Synthesis of (R)-3-(2-(3-aminopyrrolidin-1-yl) ethyl)-4-ethoxy benzonitrile hydrochloride (Compound 87)

To a stirred solution of tert-butyl (R)-(1-(5-cyano-2-ethoxyphenethyl) pyrrolidin-3-yl) carbamate (0.2 g, 0.55 mmol) in CH₂Cl₂ (2.0 mL, 10V) at 0° C., 4M HCl in dioxane (0.6 mL, 3V) was added. The reaction mixture was stirred at room temperature for 1 h. The progress of the reaction was monitored by TLC and LCMS analysis. After completion of the reaction, the reaction mixture was concentrated under reduced pressure and the crude material was triturated using diethyl ether (2×20 mL) and pentane (20 mL) to provide (R)-3-(2-(3-aminopyrrolidin-1-yl) ethyl)-4-ethoxybenzonitrile hydrochloride (0.145 g, Quantitative yield) as a brown sticky solid.

LCMS [ESI, M+1]: 210 (RT: 1.370 min, Purity: 100%)

HPLC Purity: 210 (RT: 4.086 min, Purity: 100%)

Chiral HPLC: (RT: 2.331 min, Purity: 98.97%)

Instrument Name: Waters ACQUITY UPC2

Chromatographic separation was conducted with Waters ACQUITY UPC2 system with 2998 PDA detector. The column used was CHIRALPAK® IC-3 (100×3 mm, 3 ␣m) and the compounds were eluted with Mobile Phase A: Liq·CO2, Mobile Phase B: 0.1% Methanolic ammonia in Methanol:Acetonitrile (50:50) with Gradient method; with Flow rate=2 ml/min; analysis time 5 min.

¹H NMR (400 MHz, D₂O): δ 7.60 (dd, J=8.8, 2.4 Hz, 1H), 7.51 (d, J=2.1 Hz, 1H), 7.02 (d, J=6.9 Hz, 1H), 4.13 (q, J=7.2 Hz, 3H), 3.7 (s, 3H), 3.46 (t, J=7.6 Hz, 3H), 3.01 (t, J=7.6 Hz, 2H), 2.53 (s, 1H), 2.12 (s, 1H), 1.32 (t, J=7.2 Hz, 3H).

### Experimental Protocol for Compound 88

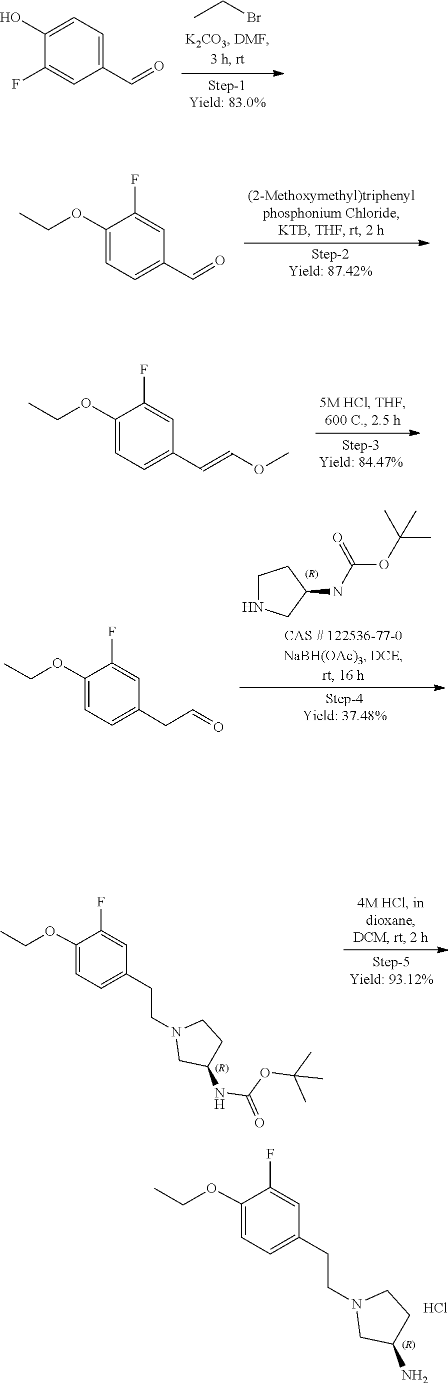

### Step-1: Synthesis of 4-ethoxy-3-fluorobenzaldehyde

To a stirred solution of 3-fluoro-4-hydroxybenzaldehyde (2.0 g, 14.2 mmol) in DMF (20 mL) at 0° C., K₂CO₃ (5.87 g, 42.6 mmol) was added. After 30 min, ethyl bromide (2.33 g, 21.4 mmol) was added at 0° C. and the resulting reaction mixture stirred at room temperature for 3 h. The progress of the reaction was monitored by TLC analysis. After completion of the reaction, the reaction mixture was poured into ice-cold water and a solid precipitate was formed. The solid material was isolated by filtration and dried under reduced pressure to provide 4-ethoxy-3-fluorobenzaldehyde (2.0 g, 83.32% yield) as an off white solid.

LCMS [ESI, M+1]: 168.8 (RT: 1.621 min, Purity: 99.71%),

¹H NMR (400 MHz, d₆-DMSO): δ 9.85 (d, J=4.0 Hz, 1H), 7.78-7.75 (m, 1H), 7.70 (dd, J=20.0, 8.0 Hz, 1H), 7.37 (t, J=8.0 Hz, 1H), 4.24 (q, J=12.0, 8.0 Hz, 2H), 1.38 (t, J=8.0 Hz, 3H).

### Step-2: Synthesis of (E)-1-ethoxy-2-fluoro-4-(2-methoxyvinyl)benzene

To a stirred solution of (2-Methoxymethyl) triphenyl phosphonium chloride (2.54 g, 7.43 mmol) in dry THF (20 mL, 40V) at 0° C., KOtBu (1M in THF; 14.8 mL, 14.8 mmol) was added. The reaction mixture was stirred at 0° C. for 1 h. Then 4-ethoxy-3-fluorobenzaldehyde (0.5 g, 2.97 mmol) was added at 0° C. and the resulting reaction mixture was stirred at 0° C. for 15 min. The progress of the reaction was monitored by TLC. After completion of the reaction, the reaction mixture was diluted with EtOAc (25 mL) and filtered through Celite®. The filtrate was concentrated under reduced pressure. The crude material was purified by column chromatography (Aluminum oxide Neutral, 100% hexane) to provide (E)-1-ethoxy-2-fluoro-4-(2-methoxyvinyl)benzene (0.51 g, 87.42%) as a colourless liquid which was used directly in the next step of the synthesis.

### Step-3: Synthesis of 2-(4-ethoxy-3-fluorophenyl)acetaldehyde

To a stirred solution of (E)-1-ethoxy-2-fluoro-4-(2-methoxyvinyl)benzene (0.51 g, 2.59 mmol) in dry THF (5.1 mL, 10V) at room temperature, 5M HCl (2.55 mL, 5V) was added. The reaction mixture was stirred at 60° C. for 2.5 h. The progress of the reaction was monitored by TLC. After completion of the reaction, the reaction mixture was poured into a saturated NaHCO₃ solution (50 mL) and extracted with EtOAc (2×50 mL). The combined organic fractions were dried over Na₂SO₄ and concentrated under reduced pressure to provide 2-(4-ethoxy-3-fluorophenyl)acetaldehyde (0.4 g, 84.47%) as a colourless liquid which was used directly in the next step of the synthesis.

### Step-4: Synthesis of tert-butyl (R)-(1-(4-ethoxy-3-fluorophenethyl)pyrrolidin-3-yl)carbamate

To a solution of 2-(4-ethoxy-3-fluorophenyl)acetaldehyde (0.4 g, 2.19 mmol) in DCE (4 mL, 10V) at room temperature, tert-butyl (R)-pyrrolidin-3-ylcarbamate (0.49 g, 2.63 mmol) was added. After 1.5 h, NaBH(OAc)₃ (1.39 g, 6.58 mmol) was added portion wise at 0° C. and the reaction mixture was stirred at room temperature for 16 h. The progress of the reaction was monitored by TLC analysis. After completion of the reaction, the reaction mixture was diluted with water (40 mL) and extracted with CH₂Cl₂ (2×50 mL). The combined organic fractions were dried over Na₂SO₄ and concentrated under reduced pressure. The crude material was purified by normal phase flash column chromatography (4% MeOH in CH₂Cl₂) to provide tert-butyl (R)-(1-(4-ethoxy-3-fluorophenethyl)pyrrolidin-3-yl)carbamate (0.29 g, 39.03%) as a brown solid.

LCMS [ESI, M, M+2]: 353.1, 354.2 (RT: 1.311 min, Purity: 91.66%)

### Step-5: Synthesis of (R)-1-(4-ethoxy-3-fluorophenethyl)pyrrolidin-3-amine hydrochloride (Compound 88)

To a stirred solution of tert-butyl (R)-(1-(4-ethoxy-3-fluorophenethyl)pyrrolidin-3-yl)carbamate (0.24 g, 0.68 mmol) in CH₂Cl₂ (2.4 mL, 10V) at 0° C., 4M HCl in Dioxane (1.25 mL, 5V) was added. The reaction mixture was stirred at room temperature for 2 h. The progress of the reaction was monitored by TLC. After completion of the reaction, the reaction mixture was diluted with diethyl ether (15 mL) and concentrated under reduced pressure to provide crude material which was purified by trituration using diethyl ether (2×10 mL), DCM (5 mL) and pentane (5 mL) to provide (R)-1-(4-ethoxy-3-fluorophenethyl)pyrrolidin-3-amine hydrochloride (0.16 g, 93.12%) as a brown sticky solid.

LCMS [ESI, M+1]: 253.0 (RT: 0.909 min, Purity: 98.57%),

HPLC Purity: RT: 4.334 min, Purity: 100%

Chiral HPLC Purity: RT: 1.765, Purity: 98.81%

Instrument Name: Waters ACQUITY UPC2

Chromatographic separation was conducted with Waters ACQUITY UPC2 system with 2998 PDA detector. The column used was CHIRALPAK® IG-3 (100×3 mm, 3 ␣m) and the compounds were eluted with Mobile Phase A: Liq·CO2, Mobile Phase B: 0.1% Methanolic ammonia in Methanol:Acetonitrile (50:50) with Gradient method; with Flow rate=2 ml/min; analysis time 4 min.

¹H NMR (400 MHz, CD₃OD): δ 7.14-7.06 (m, 3H), 4.20 (bs, 1H), 4.16-4.07 (m, 3H), 3.94-3.79 (m, 2H), 3.69-3.59 (m, 2H), 3.51-3.49 (m, 2H), 3.15-3.06 (m, 2H), 2.70-2.54 (m, 1H), 2.27-2.23 (m, 1H), 1.41 (t, J=6.8 Hz, 3H).

### Experimental Protocol for Compound 89

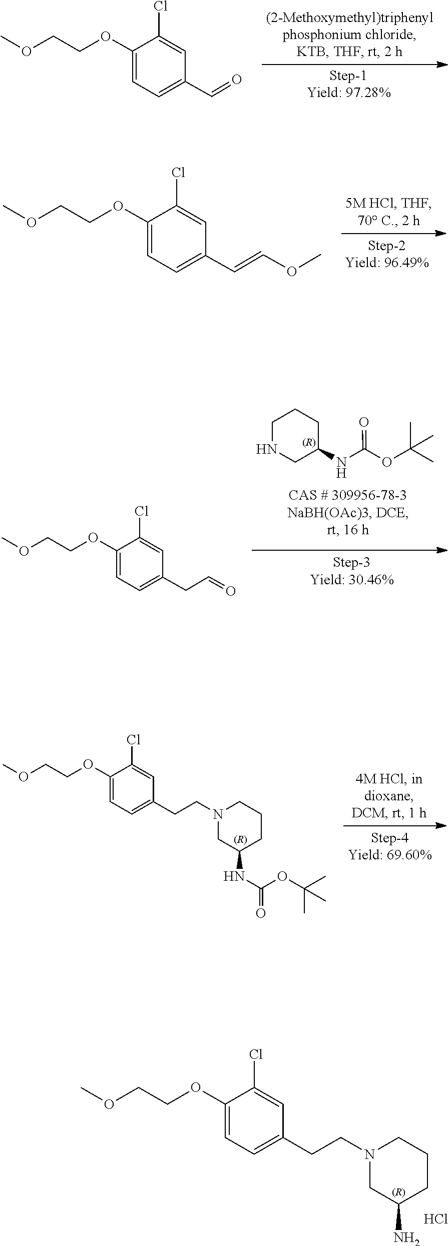

### Step-1: Synthesis of (E)-2-chloro-1-(2-methoxyethoxy)-4-(2-methoxyvinyl) benzene

To a stirred solution of (Methoxymethyl) triphenyl phosphonium chloride (4.52 g, 11.67 mmol) in dry THF (40 mL, 20V) at 0° C., potassium tert-butoxide 1M in THF (23.36 mL, 23.35 mmol) was added. Then the reaction mixture was stirred at room temperature for 1.5 h. Then, 3-chloro-4-(2-methoxyethoxy) benzaldehyde (1.0 g, 4.67 mmol) was added at 0° C. and the resulting reaction mixture was stirred at room temperature for 15 min. For the synthesis of 3-chloro-4-(2-methoxyethoxy) benzaldehyde, see step 1 of the synthesis of compound 95 below. The progress of the reaction was monitored by TLC. After completion of the reaction, the reaction mixture was diluted with EtOAc (15 mL) and filtered through Celite®. The filtrate was washed with water (2×200 mL). The organic fraction was dried over Na₂SO₄ and concentrated under reduced pressure. The crude material was purified by column chromatography (Aluminum oxide Neutral, 10% EtOAc in hexane) to provide (E)-2-chloro-1-(2-methoxyethoxy)-4-(2-methoxyvinyl) benzene (1.1 g, 97.28%) as a white sticky liquid which was used directly in the next step of the synthesis.

### Step-2: Synthesis of 2-(3-chloro-4-(2-methoxyethoxy) phenyl) acetaldehyde

To a stirred solution of (E)-2-chloro-1-(2-methoxyethoxy)-4-(2-methoxyvinyl) benzene (1.1 g, 4.54 mmol) in THF (11.1 mL, 10V) at room temperature, 5M HCl (5.6 mL, 5V) was added. The reaction mixture was stirred at 70° C. for 2 h. The progress of the reaction was monitored by TLC. After completion of the reaction, the reaction mixture was quenched by addition into a saturated NaHCO₃ solution (300 mL) and extracted with EtOAc (2×200 mL). The combined organic fractions were dried over Na₂SO₄ and concentrated under reduced pressure to provide 2-(3-chloro-4-(2-methoxyethoxy) phenyl) acetaldehyde (1.0 g, 96.49%) as a pale-yellow sticky solid which was used directly in the next step of the synthesis.

### Step-3: Synthesis of tert-butyl (R)-(1-(3-chloro-4-(2-methoxyethoxy)phenethyl)piperidin-3-yl) carbamate

To a stirred solution of 2-(3-chloro-4-(2-methoxyethoxy)phenyl)acetaldehyde (1.0 g, 4.37 mmol) in DCE (10.0 mL, 10V) at room temperature, tert-butyl (R)-piperidin-3-yl carbamate (1.04 g, 5.24 mmol) was added. After 2 h, sodium triacetoxyborohydride (2.76 g, 13.11 mmol) was added at 0° C. Then the reaction mixture was stirred at room temperature for 16 h. The progress of the reaction was monitored by TLC and LCMS analysis. After completion of the reaction, the reaction mixture was poured into a saturated NaHCO₃ solution (200 mL) and extracted with EtOAc (2×200 mL). The combined organic fractions were dried over Na₂SO₄ and concentrated under reduced pressure. The crude material was purified by reverse phase column chromatography (58% CH₃CN in water) to provide tert-butyl (R)-(1-(3-chloro-4-(2-methoxyethoxy) phenethyl) piperidin-3-yl) carbamate (0.55 g, 30.46% yield) as a pale-yellow sticky solid.

Note that two batches of this reaction were carried out in parallel on the same scale and the product yield quoted here is based on the combination of these two batches.

LCMS [ESI, M, M+2]: 413.1, 415.0 (RT: 1.355 min, Purity: 88.36%)

### Step-4: Synthesis of (R)-1-(3-chloro-4-(2-methoxyethoxy) phenethyl) piperidin-3-amine hydrochloride (Compound 89)

To a stirred solution of tert-butyl (R)-(1-(3-chloro-4-(2-methoxyethoxy) phenethyl) piperidin-3-yl) carbamate (0.35 g, 0.84 mmol) in CH₂Cl₂ (3.5 mL, 10V) at 0° C., 4M HCl in dioxane (1.75 mL, 5V) was added. The reaction mixture was stirred at room temperature for 1 h. The progress of the reaction was monitored by TLC. After completion of the reaction, the reaction mixture was concentrated under reduced pressure and the crude material was purified by reverse phase chromatography (3% CH₃CN in water) to provide (R)-1-(3-chloro-4-(2-methoxyethoxy) phenethyl) piperidin-3-amine hydrochloride (0.29 g, 69.60%) as an off-white solid.

LCMS [ESI, M, M+2]: 312.9, 314.7 (RT: 0.994 min, Purity: 99.23%),

HPLC Purity: RT: 6.02 min, Purity: 98.72%

Chiral HPLC Purity: RT: 6.95 min, Purity: 96.57%,

Instrument Name: Waters SFC Investigator

Chromatographic separation was conducted with Waters SFC Investigator system with 2998 PDA detector. The column used was Chiral ICT (250 mm×4.6 mm, 5 μm) and the compounds were eluted with, Mobile Phase A: Liq·CO2, Mobile Phase B: 0.1% Di-ethyl amine in 2-Propanol with Gradient; with Flow rate=4 ml/min; analysis time 12 min.

¹H NMR (400 MHz, D₂O): δ 7.29 (d, J=2.0 Hz, 1H), 7.11 (dd, J=8.8, 2.4 Hz, 1H), 7.00 (d, J=8.4 Hz, 1H), 4.15-4.12 (m, 2H), 3.74-3.72 (m, 3H), 3.68 (brs, 1H), 3.53 (brs, 2H), 3.36-3.35 (m, 2H), 3.34-3.32 (m, 3H), 2.96-2.92 (m, 3H), 2.14-2.00 (m, 2H), 1.76-1.69 (m, 1H), 1.58-1.52 (m, 1H)

### Experimental Protocol for Compound 90

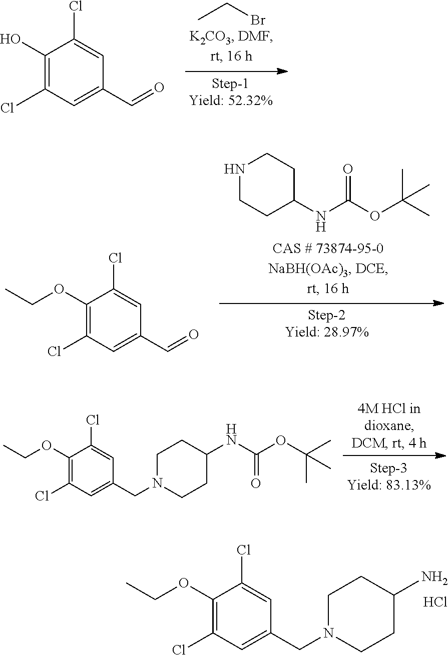

### Step-1: Synthesis of 3,5-dichloro-4-ethoxy benzaldehyde

To a stirred solution of 3,5-dichloro-4-hydroxybenzaldehyde (3.0 g, 15.7 mmol) in DMF (30 mL) at room temperature, K₂CO₃ (6.51 g, 47.1 mmol) was added. After 30 min, bromoethane (3.42 g, 31.4 mmol) was added at room temperature and the reaction mixture was stirred at room temperature for 16 h. The progress of the reaction was monitored by TLC analysis. After completion of the reaction, the reaction mixture was diluted with ice cold water (300 mL) and extracted with EtOAc (2×250 mL). The combined organic fractions were dried over Na₂SO₄ and concentrated under reduced pressure to provide 3,5-dichloro-4-ethoxybenzaldehyde (1.8 g, 52.32% yield) as a brown sticky solid.

¹H NMR (400 MHz, d₆-DMSO): δ 9.91 (s, 1H), 8.02 (s, 2H), 4.16 (q, J=14, 7.2 Hz, 2H), 1.40 (t, J=14.4 Hz, 3H).

### Step-2: Synthesis of tert-butyl (1-(3,5-dichloro-4-ethoxy benzyl) piperidin-4-yl) carbamate

To a stirred solution of 3,5-dichloro-4-ethoxy benzaldehyde (0.3 g, 1.36 mmol) in DCE (3 mL) at room temperature, tert-butyl piperidin-4-yl carbamate (0.33 g, 1.64 mmol) was added. After 1.5 h, NaBH(OAc)₃ (0.87 g, 4.10 mmol) was added at 0° C. Then the reaction mixture was stirred at room temperature for 16 h. The progress of the reaction was monitored by TLC analysis. After completion of the reaction, the reaction mixture was diluted with water (70 mL) and extracted with CH₂Cl₂ (2×70 mL). The combined organic fractions were dried over Na₂SO₄ and concentrated under reduced pressure. The crude material was purified by reverse phase column chromatography (76% CH₃CN in water) to provide tert-butyl (1-(3,5-dichloro-4-ethoxybenzyl) piperidin-4-yl) carbamate (0.16 g, 28.97% yield) as a yellow liquid.

LCMS [ESI, M, M+2]: 403.0, 404.8 (RT: 1.557 min, Purity: 100%).

### Step-3: Synthesis of 1-(3,5-dichloro-4-ethoxybenzyl) piperidin-4-amine hydrochloride (Compound 90)

To a stirred solution of tert-butyl (1-(3,5-dichloro-4-ethoxybenzyl) piperidin-4-yl) carbamate (0.16 g, 0.39 mmol) in CH₂Cl₂ (2 mL) at 0° C., 4M HCl in dioxane (1 mL, 5V) was added. Then the reaction mixture was stirred at room temperature for 4 h. The progress of the reaction was monitored by TLC analysis. After completion of the reaction, the reaction mixture was concentrated under reduced pressure and the crude material was purified by trituration using diethyl ether (2×10 mL) to provide 1-(3,5-dichloro-4-ethoxybenzyl) piperidin-4-amine hydrochloride (0.1 g, 83.13% yield) as a white solid.

LCMS [ESI, M−1, M+1]: 302.8, 304.7 (RT: 1.119 min, Purity: 100%),

HPLC Purity: RT: 3.279 min, Purity: 95.92%,

¹H NMR (400 MHz, CD₃OD): δ 7.69 (s, 2H), 4.32 (s, 2H), 4.15 (q, J=14, 6.8 Hz, 2H), 3.61 (d, J=12.0 Hz, 2H), 3.50-3.49 (m, 1H), 3.18 (t, J=25.6 Hz, 2H), 2.31 (d, J=16 Hz, 2H), 2.04-2.01 (m, 2H), 1.46 (t, J=14 Hz, 3H).

### Experimental Protocol for Compound 91

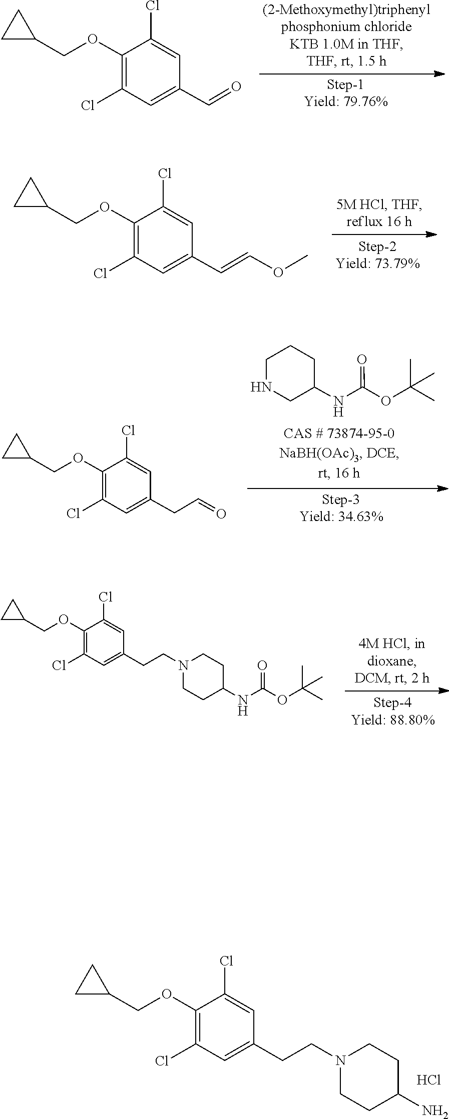

### Step-1: Synthesis of (E)-1,3-dichloro-2-(cyclopropyl methoxy)-5-(2-methoxyvinyl) benzene

To a stirred solution of (2-Methoxymethyl) triphenyl phosphonium chloride (1.57 g, 4.50 mmol) in dry THF (9 mL, 20V) at 0° C., 1M KOtBu in THF (9 mL, 9.17 mmol) was added. After 1 h, 3,5-dichloro-4-(cyclopropyl methoxy) benzaldehyde (0.45 g, 1.83 mmol) was added at 0° C. and the reaction mixture was stirred at room temperature for 20 min. The progress of the reaction was monitored by TLC analysis. After completion of the reaction, the reaction mixture was diluted with EtOAc (8 mL) and filtered through Celite®. The filtrate was concentrated under reduced pressure and the crude material was purified by column chromatography (Aluminum oxide Neutral, 100% hexane) to provide (E)-1,3-dichloro-2-(cyclopropylmethoxy)-5-(2-methoxyvinyl) benzene (0.4 g, 79.76% yield) as white sticky solid.

### Step-2: Synthesis of 2-(3,5-dichloro-4-(cyclopropyl methoxy) phenyl) acetaldehyde

To a stirred solution of (E)-1,3-dichloro-2-(cyclopropyl methoxy)-5-(2-methoxyvinyl) benzene (0.4 g, 1.46 mmol) in THF (4 mL, 10V) at room temperature, 5M aqueous HCl (2 mL, 5V) was added. The reaction mixture was stirred at room temperature for 16 h. The progress of the reaction was monitored by TLC. After completion of the reaction, the reaction mixture was quenched with sat. NaHCO₃ solution (40 mL) and extracted with EtOAc (2×40 mL). The combined organic fractions were dried over Na₂SO₄ and concentrated under reduced pressure to provide 2-(3,5-dichloro-4-(cyclopropyl methoxy) phenyl) acetaldehyde (0.28 g, 73.79%) as a yellow liquid which was used directly in the next step of the synthesis.

### Step-3: Synthesis of tert-butyl (1-(3,5-dichloro-4-(cyclopropyl methoxy) phenethyl) piperidin-4-yl) carbamate

To a stirred solution of 2-(3,5-dichloro-4-(cyclopropyl methoxy) phenyl) acetaldehyde (0.27 g, 1.04 mmol) in DCE (2.7 ml, 10V) at room temperature, tert-butyl piperidin-4-yl carbamate (0.25 g, 1.20 mmol) was added. The reaction mixture was stirred at room temperature for 2 h. Then, NaBH (OAc)₃ (0.66 g, 3.1 mmol) was added at room temperature and the reaction mixture was stirred at room temperature for 16 h. The progress of the reaction was monitored by TLC analysis. After completion of the reaction, the reaction mixture was poured into water (50 mL) and extracted with CH₂Cl₂ (3×50 mL). The combined organic fractions were dried over Na₂SO₄ and concentrated under reduced pressure. The crude material was purified by reverse phase flash column chromatography (89% CH₃CN in water) to provide tert-butyl (1-(3,5-dichloro-4-(cyclopropyl methoxy) phenethyl) piperidin-4-yl) carbamate (0.16 g, 34.63%) as a yellow sticky solid.

LCMS [ESI, M, M+2]: 443.2, 445.2 (RT: 2.013 min, Purity: 96.90%)

### Step-4: Synthesis 1-(3,5-dichloro-4-(cyclopropyl methoxy) phenethyl) piperidin-4-amine hydrochloride (Compound 91)

To a stirred solution of tert-butyl (1-(3,5-dichloro-4 (cyclopropyl methoxy) phenethyl) piperidin-4-yl) carbamate (0.16 g, 0.36 mmol) in CH₂Cl₂ (1.6 mL, 10V) at 0° C., 4M HCl in Dioxane (0.8 mL, 5V) was added. The reaction mixture was stirred at room temperature for 2 h. The progress of the reaction was monitored by TLC. After completion of the reaction, the reaction mixture was concentrated under reduced pressure and azeotroped with CH₂Cl₂ (3×10 mL) to provide crude material, which was purified by trituration using EtOAc (10 mL) to provide 1-(3,5-dichloro-4-(cyclopropyl methoxy) phenethyl) piperidin-4-amine hydrochloride (0.11 g, 88.80%) as a white solid.

LCMS [ESI, M, M+2]: 342.7, 344.6 (RT: 1.241 min, Purity: 100%),

HPLC Purity: RT: 3.525 min, Purity: 100%,

¹H NMR (400 MHz, D₂O): δ 7.25 (s, 2H), 3.82 (d, J=7.2 Hz, 2H), 3.63 (brs, 2H), 3.49-3.46 (m, 1H), 3.28-3.27 (m, 2H), 3.05 (brs, 2H), 2.94 (t, J=7.6 Hz, 2H), 2.23 (d, J=14 Hz, 2H), 1.86-1.83 (m, 2H), 1.26-1.24 (m, 1H), 0.51 (d, J=6.8 Hz, 2H), 0.24 (d, J=4.4 Hz, 2H)

### Experimental Protocol for Compound 92

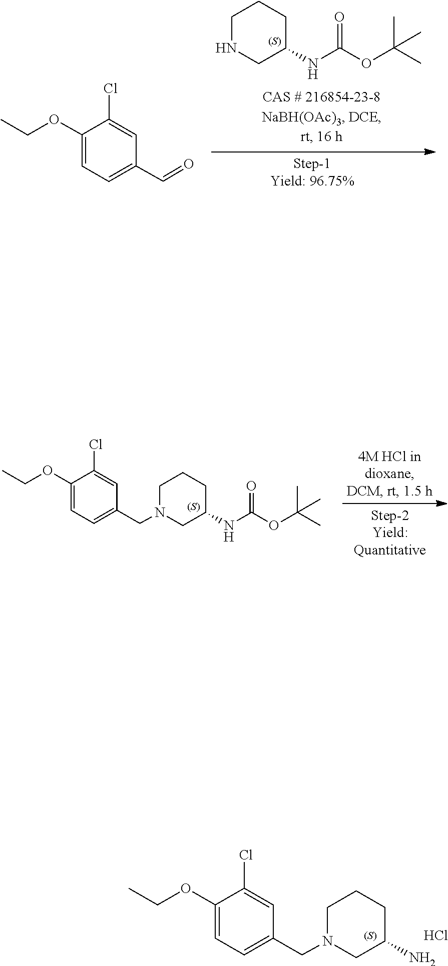

### Step-1: Synthesis of tert-butyl (S)-(1-(3-chloro-4-ethoxybenzyl)piperidin-3-yl)carbamate

To a stirred solution of 3-chloro-4-ethoxy benzaldehyde (0.3 g, 1.62 mmol) in DCE (3 mL) at room temperature, tert-butyl (S) piperidin-4-yl carbamate (0.39 g, 1.94 mmol) was added. After 1.5 h, NaBH (OAc)₃ (1.03 g, 4.87 mol) was added portion wise at 0° C. Then the reaction mixture was stirred at room temperature for 16 h. The progress of the reaction was monitored by TLC analysis. After completion of the reaction, the reaction mixture was diluted with water (50 mL) and extracted with CH₂Cl₂ (2×50 mL). The combined organic fractions were dried over Na₂SO₄ and concentrated under reduced pressure. The crude material was purified by column chromatography (SiO₂; 4% MeOH in CH₂Cl₂) to provide tert-butyl (S)-(1-(3-chloro-4-ethoxy benzyl) piperidin-3-yl) carbamate (0.58 g, 96.75%) as an off-white sticky liquid.

LCMS [ESI, M, M+2]: 369.3, 371.3 (RT: 1.606 min, Purity: 98.11%),

### Step-2: Synthesis of (S)-1-(3-chloro-4-ethoxy benzyl) piperidin-3-amine hydrochloride (Compound 92)

To a stirred solution of tert-butyl (S) (1-(3-chloro-4-ethoxybenzyl) piperidin-4-yl) carbamate (0.58 g, 0.15 mmol) in CH₂Cl₂ (5.8 mL) at 0° C., 4M HCl in Dioxane (2.9 mL, 5V) was added. Then the reaction mixture was stirred at room temperature for 1.5 h. The progress of the reaction was monitored by TLC analysis. After completion of the reaction, the reaction mixture was concentrated under reduced pressure and the crude material was purified by trituration using diethyl ether (2×10 mL) to provide (S)-1-(3-chloro-4-ethoxy benzyl) piperidin-3-amine hydrochloride (0.43 g, Quantitative yield) as an off-white solid.

LCMS [ESI, M, M+2]: 268.9, 270.7 (RT: 0.809 min, Purity: 100%),

HPLC Purity: RT: 3.70 min, Purity: 100%,

Chiral HPLC Purity: RT: 3.48 min, Purity: 100%

Instrument Name: Waters SFC Investigator

Chromatographic separation was conducted with Waters SFC Investigator system with 2998 PDA detector. The column used was Chiral ICT (250 mm×4.6 mm, 5 μm) and the compounds were eluted with, Mobile Phase A: Liq·CO2, Mobile Phase B: 0.1% Di-ethyl amine in 2-Propanol:Acetonitrile (70:30) with Gradient; with Flow rate=4 ml/min; analysis time 08 min

¹H NMR (400 MHz, D₂O): δ 7.44 (s, 1H), 7.28 (d, J=8.4 Hz, 1H), 7.08 (d, J=8.8 Hz, 1H), 4.18-4.09 (m, 4H), 3.56-3.35 (m, 3H), 2.88-2.82 (m, 2H), 2.09 (d, J=11.6 Hz, 1H), 1.97 (d, J=14.8 Hz, 1H), 1.68-1.50 (m, 2H), 1.30 (t, J=6.4 Hz, 3H).

### Experimental Protocol for Compound 93

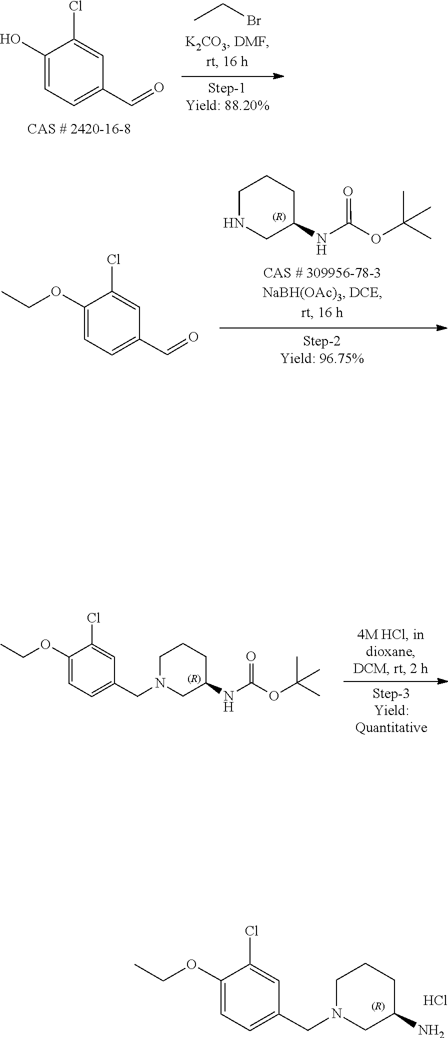

### Step-1: Synthesis of 3-chloro-4-ethoxybenzaldehyde

To a stirred solution of 3-chloro-4-hydroxybenzaldehyde (5.0 g, 31.9 mmol) in DMF (50 mL) at room temperature, K₂CO₃ (13.24 g, 95.8 mmol) was added. After 25 min, ethyl bromide (4.17 g, 38.3 mmol) was added dropwise wise at room temperature and the reaction mixture was stirred at room temperature for 16 h. The progress of the reaction was monitored by TLC analysis. After completion of the reaction, the reaction mixture was filtered and the solid material dried under reduced pressure to provide 3-chloro-4-ethoxybenzaldehyde (5.2 g, 88.20% yield) as an off-white solid.

¹H NMR (400 MHz, d₆-DMSO): δ 9.86 (S, 1H), 7.94 (d, J=2.0 Hz, 1H), 7.89-7.86 (dd, J 2.0, 8.4, 1H), 7.35 (d, J=8.4, 1H), 4.27-4.22 (m, 2H), 1.39 (t, J=7.2, 3H).

### Step-2: Synthesis of tert-butyl (R)-(1-(3-chloro-4-ethoxybenzyl) piperidin-3-yl) carbamate

To a stirred solution of 3-chloro-4-ethoxybenzaldehyde (0.3 g, 1.62 mmol) in DCE (3 mL) at room temperature, tert-butyl (R) piperidin-4-yl carbamate (0.39 g, 1.94 mmol) was added. After 1.5 h, NaBH(OAc)₃ (1.03 g, 4.87 mol) was added portion wise at 0° C. Then the reaction mixture was stirred at room temperature for 16 h. The progress of the reaction was monitored by TLC analysis. After completion of the reaction, the reaction mixture was diluted with water (50 mL) and extracted with CH₂Cl₂ (2×50 mL). The combined organic fractions were dried over Na₂SO₄ and concentrated under reduced pressure. The crude material was purified by column chromatography (SiO₂; 3% MeOH in CH₂Cl₂) to provide tert-butyl (R)-(1-(3-chloro-4-ethoxybenzyl) piperidin-3-yl) carbamate (0.58 g, 96.75%) as an off-white sticky liquid.

LCMS [ESI, M, M+2]: 369.1, 371.0 (RT: 1.442 min, Purity: 100%),

### Step-3: Synthesis of (R)-1-(3-chloro-4-ethoxybenzyl) piperidin-3-amine hydrochloride (Compound 93)

To a stirred solution of tert-butyl (R) (1-(3-chloro-4-ethoxybenzyl) piperidin-4-yl) carbamate (0.5 g, 0.1.35 mmol) in CH₂Cl₂ (5 mL) at 0° C., 4M HCl in dioxane (2.5 mL, 5V) was added. The reaction mixture was stirred at room temperature for 2 h. The progress of the reaction was monitored by TLC analysis. After completion of the reaction, the reaction mixture was concentrated under reduced pressure and the crude material was purified by trituration using diethyl ether (2×10 mL) to provide (R)-1-(3-chloro-4-ethoxybenzyl) piperidin-3-amine hydrochloride (0.46 g, Quantitative Yield) as an off-white solid.

LCMS [ESI, M, M+2]: 268.9, 270.7 (RT: 0.811 min, Purity: 100%),

HPLC Purity: RT: 4.274 min, Purity: 100%,

Chiral HPLC Purity: RT: 2.40 min, Purity: 100%

Instrument Name: Waters SFC Investigator

Chromatographic separation was conducted with Waters SFC Investigator system with 2998 PDA detector. The column used was Chiral ICT (250 mm×4.6 mm, 5 μm) and the compounds were eluted with, Mobile Phase A: Liq·CO2, Mobile Phase B: 0.1% Di-ethyl amine in 2-Propanol:Acetonitrile (70:30) with Gradient; with Flow rate=4 ml/min; analysis time 08 min.

¹H NMR (400 MHz, D₂O): δ 7.44 (d, J=2 Hz, 1H), 7.28 (dd, J=8.4, 2.0 Hz, 1H), 7.08 (d, J=8.4 Hz, 1H), 4.35-4.09 (m, 4H), 3.55-3.34 (m, 3H), 2.99-2.82 (m, 2H), 2.09 (d, J=12.8 Hz, 1H), 1.96 (d, J=14.8 Hz, 1H), 1.68-1.56 (m, 1H), 1.52-1.46 (m, 1H), 1.31 (t, J=6.8 Hz, 3H).

### Experimental Protocol for Compound 94

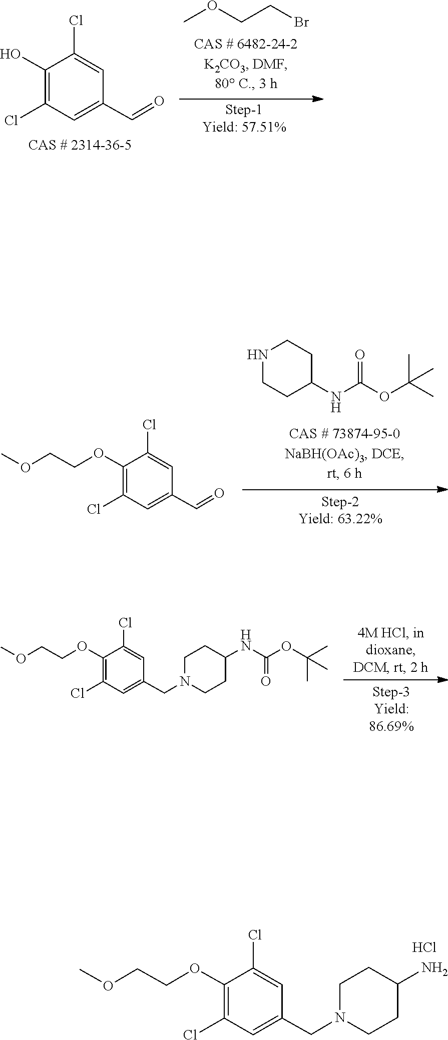

### Step-1: Synthesis of 3,5-dichloro-4-(2-methoxyethoxy)benzaldehyde

To a stirred solution of 3,5-dichloro-4-hydroxybenzaldehyde (1.0 g, 5.23 mmol) in DMF (10 mL, 10V) at room temperature, K₂CO₃ (1.45 g, 13.08 mmol) was added. After 15 min, 1-bromo-2-methoxyethane (1.09 g, 7.85 mmol) was added at room temperature and the reaction mixture was stirred 80° C. for 3 h. The progress of the reaction was monitored by TLC analysis. After completion of the reaction, the reaction mixture was poured into water (30 mL) and extracted with EtOAc (3×30 mL). The combined organic fractions were dried over Na₂SO₄ and concentrated under reduced pressure to provide 3,5-dichloro-4-(2-methoxyethoxy)benzaldehyde (0.75 g, 57.51%) as a brown sticky liquid. Note that two batches of this reaction were carried out all on the same scale and the product yield quoted here is based on the combination of these two batches.

¹H NMR (400 MHz, d₆-DMSO): δ 9.91 (s, 1H), 8.01 (s, 2H), 4.2-4.24 (m, 2H), 3.73-3.69 (m, 2H), 3.30 (s, 3H).

### Step-2: Synthesis of tert-butyl (1-(3,5-dichloro-4-(2-methoxyethoxy) benzyl) piperidin-4-yl) carbamate

To a stirred solution of 3,5-dichloro-4-(2-methoxyethoxy) benzaldehyde (0.3 g, 1.20 mmol) in DCE (3 mL, 10V) at room temperature, tert-butyl piperidin-4-yl carbamate (0.32 g, 1.44 mmol) and AcOH (0.01 ml, 0.05V) were added. After 1.5 h, sodium triacetoxyborohydride (0.76 g, 3.61 mmol) was added portion-wise at 0° C. and then the reaction mixture was allowed to stir at room temperature for 6 h. The progress of the reaction was monitored by TLC and LCMS analysis. After completion of the reaction, the reaction mixture was poured into a sat. NaHCO₃ solution (30 mL) and extracted with CH₂Cl₂ (3×20 mL). The combined organic fractions were dried over Na₂SO₄ and concentrated under reduced pressure to provide tert-butyl (1-(3,5-dichloro-4-(2-methoxyethoxy) benzyl) piperidin-4-yl) carbamate (0.33 g, 63.22% yield) as a yellow viscous liquid.

LCMS [ESI, M, M+2]: 433.0, 434.9 (RT: 1.403 min, Purity: 97.06%)

### Step-3: Synthesis of 1-(3,5-dichloro-4-(2-methoxyethoxy) benzyl) piperidin-4-amine hydrochloride (Compound 94)

To a stirred solution of tert-butyl (1-(3,5-dichloro-4-(2-methoxyethoxy)benzyl)piperidin-4-yl)carbamate (0.33 g, 0.83 mmol) in CH₂Cl₂ (3.3 mL, 10V) at 0° C., 4M HCl in Dioxane (1.65 mL, 5V) was added. The reaction mixture was then stirred at room temperature for 2 h. The progress of the reaction was monitored by TLC analysis. After completion of the reaction, the reaction mixture was concentrated under reduced pressure and azeotroped with CH₂Cl₂ to provide crude material, which was triturated using CH₂Cl₂ (2 mL) and diethyl ether (2×15 mL) to provide 1-(3,5-dichloro-4-(2-methoxyethoxy) benzyl) piperidin-4-amine hydrochloride (0.22 g, 86.69% yield) as an off-white sticky solid.

LCMS [ESI, M, M+1]: 332.9, 334.7 (RT: 0.875 min, Purity: 100%),

HPLC Purity: RT: 4.290 min, Purity: 99.52%

¹H NMR (400 MHz, D₂O): δ 7.45 (s, 2H), 4.18 (s, 4H), 3.76-3.74 (m, 2H), 3.50-3.43 (m, 3H), 3.33 (s, 3H), 3.03 (t, J=10.8 Hz, 2H), 2.20 (d, J=14.2 Hz, 2H), 1.81-1.78 (m, 2H).

### Experimental Protocol for Compound 95

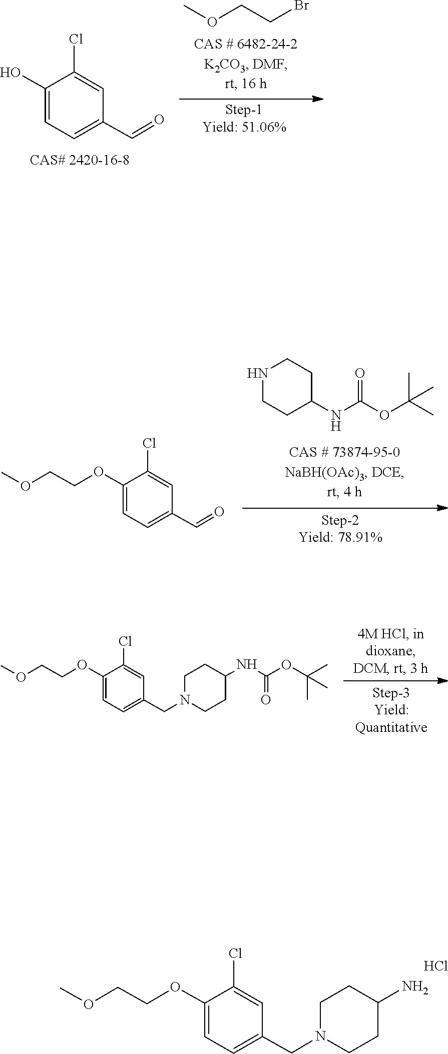

### Step-1: Synthesis of 3-chloro-4-(2-methoxyethoxy)benzaldehyde

To a stirred solution of 3-chloro-4-hydroxybenzaldehyde (1.0 g, 6.38 mmol) in DMF (10 mL) at room temperature, K₂CO₃ (2.2 g, 15.9 mmol) was added. After 15 min, 1-bromo-2-methoxyethane (1.15 g, 8.30 mmol) was added at room temperature and the reaction mixture was stirred at room temperature for 16 h. The progress of the reaction was monitored by TLC analysis. After completion of the reaction, the reaction mixture was diluted with ice cold water (100 mL) and extracted with EtOAc (2×50 ml). The combine organic fractions were dried over Na₂SO₄ and concentrated under reduced pressure to provide 3-chloro-4-(2-methoxyethoxy)benzaldehyde (0.7 g, 51.06% yield) as a light brown solid.

¹H NMR (400 MHz, d₆-DMSO): δ 9.86 (s, 1H), 7.96 (d, J=1.6 Hz, 1H), 7.88 (dd, J=8.4, 2 Hz, 1H), 7.38 (d, J=8.8 Hz, 1H), 4.32 (t, J=4.4 Hz, 2H), 3.74-3.71 (m, 2H). (Note: 3 protons of —OCH₃ are assumed to merge with d₆-DMSO moisture peak).

### Step-2: Synthesis of tert-butyl (1-(3-chloro-4-(2-methoxyethoxy) benzyl) piperidin-4-yl) carbamate

To a stirred solution of 3-chloro-4-(2-methoxyethoxy) benzaldehyde (0.3 g, 1.39 mmol) in DCE (3 mL) at room temperature, tert-butyl piperidin-4-yl carbamate (0.33 g, 1.67 mmol) was added. After 1.5 h, NaBH(OAc)₃ (0.88 g, 4.19 mmol) was added portion wise at 0° C. Then the reaction mixture was stirred at room temperature for 4 h. The progress of the reaction was monitored by TLC analysis. After completion of the reaction, the reaction mixture was diluted with water (50 mL) and extracted with CH₂Cl₂ (2×50 mL). The combined organic fractions were dried over Na₂SO₄ and concentrated under reduced pressure. The crude material was purified by normal phase column chromatography (5% MeOH in CH₂Cl₂) to provide tert-butyl (1-(3-chloro-4-(2-methoxyethoxy) benzyl) piperidin-4-yl) carbamate (0.44 g, 78.91% yield) as a light brown liquid.

LCMS [ESI, M, M+1]: 399.11, 401.1 (RT: 1.308 min, Purity: 94.49%),

### Step-3: Synthesis of 1-(3-chloro-4-(2-methoxyethoxy) benzyl) piperidin-4-amine hydrochloride (Compound 95)

To a stirred solution of tert-butyl (1-(3-chloro-4-(2-methoxyethoxy) benzyl) piperidin-4-yl) carbamate (0.38 g, 0.95 mmol) in DCM (4.0 mL) at 0° C., 4M HCl in Dioxane (2.0 mL, 5V) was added. Then the reaction mixture was stirred at room temperature for 3 h. The progress of the reaction was monitored by TLC analysis. After completion of the reaction, the reaction mixture was concentrated under reduced pressure and the crude material purified by trituration using diethyl ether (2×10 mL) to provide 1-(3-chloro-4-(2-methoxyethoxy) benzyl) piperidin-4-amine hydrochloride (0.31 g, Yield: quantitative) as an off-white solid.

LCMS [ESI, M, M+2]: 298.8, 300.7 (RT: 0.685 min, Purity: 98.38%),

HPLC Purity: RT: 3.51 min, Purity: 98.31%,

¹H NMR (400 MHz, D₂O): δ 7.45 (d, J=1.6 Hz, 1H), 7.28 (d, J=8.8 Hz, 1H), 7.08 (d, J=8.8 Hz, 1H), 4.19-4.15 (m, 4H), 3.77-3.75 (m, 2H), 3.48-3.38 (m, 3H), 3.33 (s, 3H), 3.09-2.98 (m, 2H), 2.99 (brs, 2H), 2.18 (d, J=13.6 Hz, 2H), 1.77 (d, J=12.4 Hz, 2H).

### Experimental Protocol for Compound 96

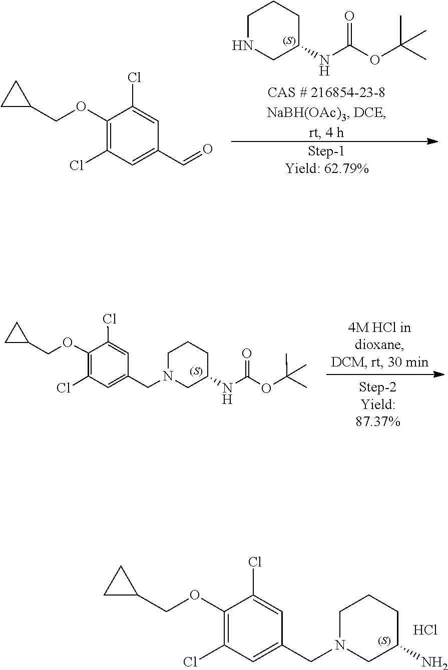

### Step-1: Synthesis of tert-butyl (S)-(1-(3,5-dichloro-4-(cyclopropyl methoxy) benzyl) piperidin-3-yl) carbamate

To a stirred solution of 3,5-dichloro-4-(cyclopropyl methoxy) benzaldehyde (0.1 g, 0.40 mmol) in DCE (1 mL) at room temperature, tert-butyl (S)-piperidin-3-yl carbamate (0.09 g, 0.44 mmol) was added. After 1H, NaBH (OAc)₃ (0.25 g, 1.22 mmol) was added at room temperature and the reaction mixture was stirred at room temperature for 3 h. The progress of the reaction was monitored by TLC analysis. After completion of the reaction, the reaction mixture was diluted with water (50 ml) and extracted with CH₂Cl₂ (2×50 ml). The combined organic fractions were dried over Na₂SO₄ and concentrated under reduced pressure to provide tert-butyl (S)-(1-(3,5-dichloro-4-(cyclopropyl methoxy) benzyl) piperidin-3-yl) carbamate (0.11 g, 62.79% yield) as a light brown solid.

LCMS [ESI, M, M+2]: 429.0, 430.9 (RT: 1.700 min, Purity: 62.27%),

### Step-2: Synthesis of (S)-1-(3,5-dichloro-4-(cyclopropyl methoxy) benzyl) piperidin-3-amine hydrochloride (Compound 96)

To a stirred solution of tert-butyl (S)-(1-(3,5-dichloro-4-(cyclopropyl methoxy) benzyl) piperidin-3-yl) carbamate (0.1 g, 0.2 mmol) in CH₂Cl₂ (1 mL) at 0° C., 4M HCl in Dioxane (0.5 mL, 5V) was added. Then the reaction mixture was stirred at room temperature for 30 min. The progress of the reaction was monitored by TLC analysis. After completion of the reaction, the reaction mixture was concentrated under reduced pressure and the crude material purified by preparative HPLC to provide (S)-1-(3,5-dichloro-4-(cyclopropyl methoxy) benzyl) piperidin-3-amine hydrochloride (0.067 g, 87.37% yield) as a white solid.

LCMS [ESI, M, M+2]: 329.2, 331.2 (RT: 3.683 min, Purity: 100%),

HPLC Purity: RT: 4.22 min, Purity: 100%,

Chiral HPLC Purity: RT: 4.43 min, Purity: 100%

Instrument Name: Waters SFC Investigator

Chromatographic separation was conducted with Waters SFC Investigator system with 2998 PDA detector. The column used was Chiral ICT (250 mm×4.6 mm, 5 μm) and the compounds were eluted with, Mobile Phase A: Liq·CO2, Mobile Phase B: 0.1% Di-ethyl amine in 2-Propanol:Acetonitrile (70:30) with Gradient; with Flow rate=4 ml/min; analysis time 08 min.

¹H NMR (400 MHz, D₂O): δ 7.43 (s, 2H), 4.16 (s, 2H), 3.87 (d, J=7.6 Hz, 2H) 3.56-3.47 (m, 2H), 3.31 (d, J=10.4 Hz, 1H), 2.90-2.78 (m, 2H), 2.09 (d, J=11.2 Hz, 1H), 1.96 (d, J=14.4 Hz, 1H), 1.72-1.62 (m, 1H), 1.56-1.50 (m, 1H), 1.28-1.26 (m, 1H), 0.51 (d, J=7.2 Hz, 2H), 0.24 (d, J=4.4 Hz, 2H)

### Experimental Protocol for Compound 97

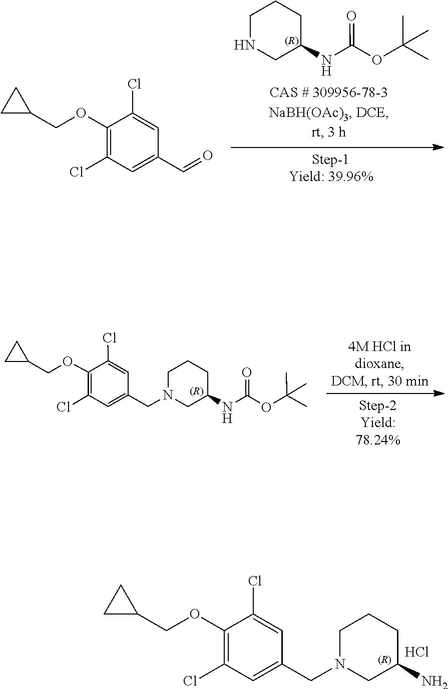

### Step-1: Synthesis of tert-butyl (R)-(1-(3,5-dichloro-4-(cyclopropyl methoxy) benzyl) piperidin-3-yl) carbamate

To a stirred solution of 3,5-dichloro-4-(cyclopropyl methoxy) benzaldehyde (0.3 g, 1.22 mmol) in DCE (3 ml, 10V) at room temperature, tert-butyl (R)-piperidin-3-yl carbamate (0.27 g, 1.3 mmol) was added. After 1H, NaBH (OAc)₃ (0.77 g, 3.6 mmol) was added at room temperature and the reaction mixture was stirred at room temperature for 3 h. The progress of the reaction was monitored by TLC analysis. After completion of the reaction, the reaction mixture was diluted with water (50 mL) and extracted with EtOAc (2×50 ml). The combined organic fractions were dried over Na₂SO₄ and concentrated under reduced pressure. The crude material was purified by column chromatography (SiO₂; 51% EtOAc in hexane) to provide tert-butyl (R)-(1-(3,5-dichloro-4-(cyclopropyl methoxy) benzyl) piperidin-3-yl) carbamate (0.21 g, 39.96% yield) as a light brown solid.

LCMS [ESI, M, M+2]: 429.0, 430.8 (RT: 1.694 min, Purity: 40.43%)

### Step-2: Synthesis of (R)-1-(3,5-dichloro-4-(cyclopropylmethoxy)benzyl)piperidin-3-amine hydrochloride (Compound 97)

To a stirred solution of tert-butyl (R)-(1-(3,5-dichloro-4-(cyclopropyl methoxy) benzyl) piperidin-3-yl) carbamate (0.2 g, 0.46 mmol) in CH₂Cl₂ (2 mL, 10V) at 0° C., 4M HCl in Dioxane (1 mL, 5V) was added. The reaction mixture was stirred at room temperature for 30 min. The progress of the reaction was monitored by TLC analysis. After completion of the reaction, the reaction mixture was concentrated under reduced pressure and the crude material purified by preparative HPLC to provide (R)-1-(3,5-dichloro-4-(cyclopropylmethoxy)benzyl)piperidin-3-amine hydrochloride (0.12 g, 78.24%) as a white solid.

LCMS [ESI, M, M+2]:329.1, 331.1 (RT: 1.431 min, Purity: 100%).

HPLC Purity: RT: 4.565 min, Purity: 97.20%,

Chiral HPLC Purity: RT: 5.62 min, Purity: 100%,

Chiral-SFC MOA

Instrument Name: Waters SFC Investigator

Chromatographic separation was conducted with Waters SFC Investigator system with 2998 PDA detector. The column used was Chiral ICT (250 mm×4.6 mm, 5 μm) and the compounds were eluted with, Mobile Phase A: Liq·CO2, Mobile Phase B: 0.1% Di-ethyl amine in 2-Propanol:Acetonitrile (70:30) with Gradient; with Flow rate=4 ml/min; analysis time 08 min.

¹H NMR (400 MHz, D₂O): δ 7.44 (s, 2H), 4.18 (s, 2H), 3.88 (d, J=7.2 Hz, 2H) 3.57-3.48 (m, 2H), 3.34 (d, J=12.4 Hz, 1H) 2.92-2.79 (m, 2H), 2.10 (d, J=11.2 Hz, 1H), 1.96 (d, J=15.2 Hz, 1H), 1.67-1.72 (m, 1H), 1.57-1.47 (m, 1H), 1.29-1.23 (m, 1H), 0.53-0.49 (m, 2H), 0.25-0.24 (m, 2H).

### Experimental Protocol for Compound 98

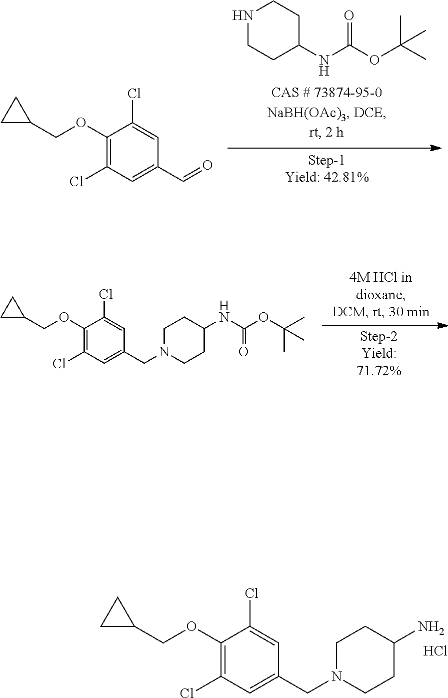 [SMILES: CC(C)(C)OC(=O)NC1CCNCC1 ; O=Cc1cc(Cl)c(OCC2CC2)c(Cl)c1 ; CC(C)(C)OC(=O)NC1CCN(Cc2cc(Cl)c(OCC3CC3)c(Cl)c2)CC1 ; NC1CCN(Cc2cc(Cl)c(OCC3CC3)c(Cl)c2)CC1 ; Cl]

### Step-1: Synthesis of tert-butyl (1-(3,5-dichloro-4-(cyclopropyl methoxy) benzyl) piperidin-4-yl) carbamate

To a stirred solution of 3,5-dichloro-4-(cyclopropyl methoxy) benzaldehyde (0.2 g, 0.18 mmol) in DCE (2 mL) at room temperature, tert-butyl piperidin-4-yl carbamate (0.17 g, 0.89 mmol) was added. After 1H, NaBH (OAc)₃ (0.51 g, 2.40 mmol) was added at room temperature. Then the reaction mixture was stirred at room temperature for 2 h. The progress of the reaction was monitored by TLC and LCMS analysis. After completion of the reaction, the reaction mixture was poured into water (50 mL) and extracted with CH₂Cl₂ (2×50 mL). The combined organic fractions were washed with a sat. NH₄Cl solution (50 mL) and a brine solution (50 mL). The combined organic fractions were dried over Na₂SO₄ and concentrated under reduced pressure. The crude material was purified by column chromatography (SiO₂; 30% EtOAc in hexane) to provide tert-butyl (1-(3,5-dichloro-4-(cyclopropyl methoxy) benzyl) piperidin-4-yl) carbamate (0.15 g, 42.81% yield) as an off-white solid.

LCMS [ESI, M, M+2]: 429.0, 430.9 (RT: 1.686 min, Purity: 86.43%).

### Step-2: Synthesis of 1-(3,5-dichloro-4-(cyclopropyl methoxy) benzyl) piperidin-4-amine (Compound 98)

To a stirred solution of tert-butyl (1-(3,5-dichloro-4-(cyclopropyl methoxy) benzyl) piperidin-4-yl) carbamate (0.2 g, 46.5 mmol) in CH₂Cl₂ (2 mL, 10V) at 0° C., 4M HCl in Dioxane (1 mL, 5V) was added. The the reaction mixture was stirred at room temperature for 30 min. The progress of the reaction was monitored by TLC analysis. After completion of the reaction, the reaction mixture was concentrated under reduced pressure and the crude material purified by trituration using diethyl ether (10 mL) and n-pentane (5 mL) to provide 1-(3,5-dichloro-4-(cyclopropyl methoxy) benzyl) piperidin-4-amine (0.11 g, 71.72%) as an off white solid.

LCMS [ESI, M+, M+2]: 329.2, 331.2 (RT: 5.031 min, Purity: 95.16%),

HPLC Purity: RT: 4.870 min, Purity: 95.18%,

1H NMR (400 MHz, D₂O): δ 7.44 (s, 2H), 4.16 (s, 2H), 3.88 (d, J=3.2 Hz, 2H), 3.48-3.40 (m, 3H), 3.03 (t, J=12.4 Hz, 2H), 2.19 (d, J=13.2 Hz, 2H), 1.81-1.76 (m, 2H), 1.30-1.24 (m, 1H), 0.54-0.50 (m, 2H) 0.26-0.25 (m, 2H).

### Experimental Protocol for Compound 99

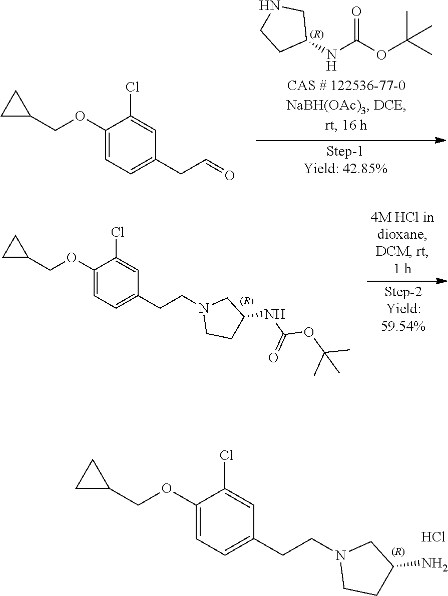

### Step-1: Synthesis of tert-butyl (R)-(1-(3-chloro-4-(cyclopropyl methoxy) phenethyl) pyrrolidin-3-yl) carbamate

To a stirred solution of 2-(3-chloro-4-(cyclopropyl methoxy) phenyl) acetaldehyde (0.6 g, 2.67 mmol) in DCE (6.0 mL) at room temperature, tert-butyl (R)-pyrrolidin-3-yl carbamate (0.59 g, 3.20 mmol) was added. After 1 h, sodium triacetoxyborohydride (1.69 g, 8.01 mmol) was added at 0° C. and the resulting reaction mixture was stirred at room temperature for 16 h. The progress of the reaction was monitored by TLC analysis. After completion of the reaction, the reaction mixture was poured in water (70 mL) and extracted with CH₂Cl₂ (2×70 mL). The combined organic fractions were dried over Na₂SO₄ and concentrated under reduced pressure. The crude material was purified by reverse phase column chromatography (65% CH₃CN in water) to provide tert-butyl (R)-(1-(3-chloro-4-(cyclopropyl methoxy) phenethyl) pyrrolidin-3-yl) carbamate (0.45 g, 42.85%) as a yellow oil.

LCMS [ESI, M, M+2]: 395.3, 397.3 (RT: 1.808 min, Purity: 96.25%),

### Step-2: Synthesis of (R)-1-(3-chloro-4-(cyclopropyl methoxy) phenethyl) pyrrolidin-3-amine hydrochloride

To a stirred solution tert-butyl (R)-(1-(3-chloro-4-(cyclopropylmethoxy)phenethyl)pyrrolidin-3-yl) carbamate (0.45 g, 1.13 mmol) in CH₂Cl₂ (5.0 mL) at 0° C., 4M HCl in Dioxane (2.5 mL) was added. The reaction mixture was stirred at room temperature for 1 h. The progress of the reaction was monitored by TLC analysis. After completion of the reaction, the reaction mixture was concentrated under reduced pressure and the crude material purified by reverse phase column chromatography (100% water) to provide (R)-1-(3-chloro-4-(cyclopropylmethoxy)phenethyl)pyrrolidin-3-amine hydrochloride (0.2 g, 59.54% yield) as an off-white sticky solid.

LCMS [ESI, M, M+2]: 294.9, 296.7 (RT: 1.045 min, Purity: 99.03%),

HPLC Purity: RT: 4.290 min, Purity: 95.92%,

Chiral HPLC Purity: RT: 6.41 min, Purity: 100%

Instrument Name: Waters SFC Investigator

Chromatographic separation was conducted with Waters SFC Investigator system with 2998 PDA detector. The column used was Chiral ICT (250 mm×4.6 mm, 5 μm) and the compounds were eluted with, Mobile Phase A: Liq·CO2, Mobile Phase B: 0.1% Di-ethyl amine in 2-Propanol:Acetonitrile (70:30) with Gradient; with Flow rate=4 ml/min; analysis time 12 min.

¹H NMR (400 MHz, D₂O) δ 7.31 (s, 1H), 7.12 (d, J=8.4 Hz, 1H), 7.01 (d, J=8.4 Hz, 1H), 4.69 (s, 2H), 4.09 (brs, 1H), 3.85 (d, J=7.2 Hz, 3H), 3.46 (t, J=8.0 Hz, 3H), 2.93 (t, J=7.2 Hz, 2H), 2.52 (brs, 1H), 2.10 (brs, 1H), 1.22-1.15 (m, 1H), 0.54-0.50 (m, 2H), 0.26-0.22 (m, 2H).

### Experimental Protocol for Compound 100

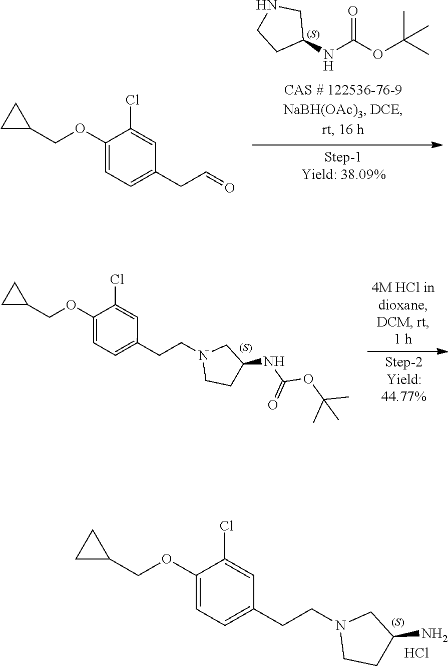

### Step-1: Synthesis of tert-butyl (S)-(1-(3-chloro-4-(cyclopropyl methoxy) phenethyl) pyrrolidin-3-yl) carbamate

To a stirred solution of 2-(3-chloro-4-(cyclopropyl methoxy) phenyl) acetaldehyde (0.6 g, 2.67 mmol) in DCE (6 mL) at room temperature, tert-butyl (S)-pyrrolidin-3-yl carbamate (0.59 g, 3.20 mmol) was added. After 1 h, sodium tri acetoxy borohydride (1.69 g, 8.01 mmol) was added at 0° C. and the resulting reaction mixture was stirred at room temperature for 16 h. The progress of the reaction was monitored by TLC analysis. After completion of the reaction, the reaction mixture was poured in water (70 mL) and extracted with CH₂Cl₂ (3×50 mL). The combined organic fractions were dried over Na₂SO₄ and concentrated under reduced pressure. The crude material was purified by reverse phase column chromatography (63% CH₃CN in water) to provide tert-butyl (S)-(1-(3-chloro-4-(cyclopropyl methoxy) phenethyl) pyrrolidin-3-yl) carbamate (0.4 g, yield 37.93%) as a yellow oil.

LCMS [ESI, M, M+2]: 395.3, 397.3 (RT: 1.847 min, Purity: 96.74%),

### Step-2: Synthesis of (S)-1-(3-chloro-4-(cyclopropylmethoxy)phenethyl)pyrrolidin-3-amine hydrochloride (Compound 100)

To a stirred solution tert-butyl (S)-(1-(3-chloro-4-(cyclopropylmethoxy)phenethyl)pyrrolidin-3-yl) carbamate (0.4 g, 1.01 mmol) in CH₂C₁₂ (4.0 mL) at 0° C., 4M HCl in dioxane (2.0 mL) was added. The reaction mixture was stirred at room temperature for 1 h. The progress of the reaction was monitored by TLC analysis. After completion of the reaction, the reaction mixture was concentrated under reduced pressure and the crude material purified by reverse phase column chromatography (100% water) to provide (S)-1-(3-chloro-4-(cyclopropyl methoxy) phenethyl) pyrrolidin-3-amine hydrochloride (0.2 g, 44.77% yield) as an off-white sticky solid.

LCMS [ESI, M, M+2]: 294.9, 296.7 (RT: 1.056 min, Purity: 96.42%),

HPLC Purity: RT: 4.269 min, Purity: 95.19%,

Chiral HPLC Purity: RT: 6.95 min, Purity: 100%

Instrument Name: Waters SFC Investigator

Chromatographic separation was conducted with Waters SFC Investigator system with 2998 PDA detector. The column used was Chiral ICT (250 mm×4.6 mm, 5 μm) and the compounds were eluted with, Mobile Phase A: Liq·CO2, Mobile Phase B: 0.1% Di-ethyl amine in 2-Propanol:Acetonitrile (70:30) with Gradient; with Flow rate=4 ml/min; analysis time 12 min.

¹H NMR (400 MHz, D₂O) δ 7.31 (d, J=2.0 Hz, 1H), 7.12 (dd, J=8.4, 2.0 Hz, 1H), 7.01 (d, J=8.4 Hz, 1H), 4.07 (brs, 1H), 3.85 (d, J=7.2 Hz, 3H), 3.42 (t, J=7.2 Hz, 3H), 2.91 (t, J=7.6 Hz, 2H), 2.51 (brs, 1H), 2.08 (brs, 1H), 1.27-1.18 (m, 1H), 0.59-0.55 (m, 2H), 0.35-0.32 (m, 2H).

### Experimental Protocol for Compound 101

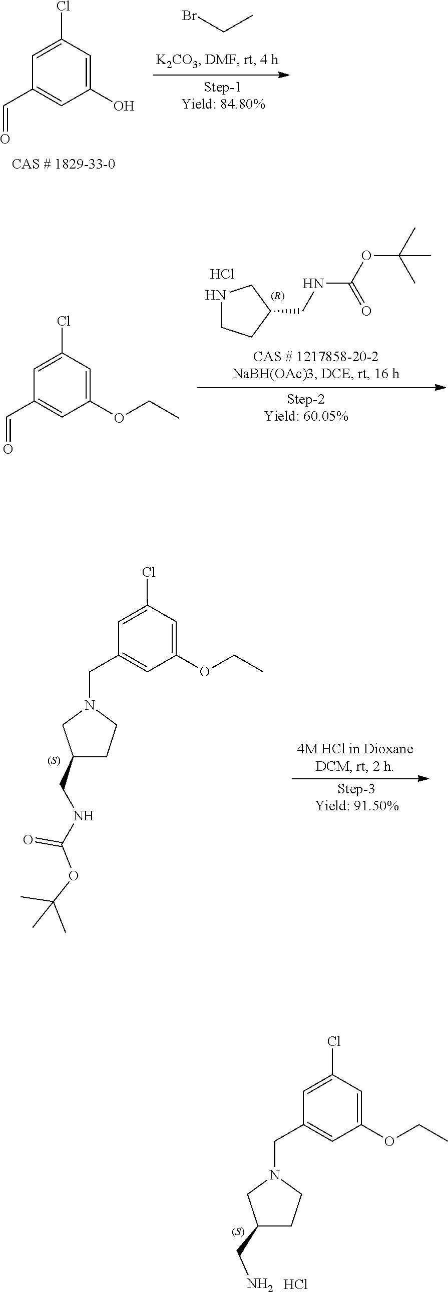

### Step-1: Synthesis of 3-chloro-5-ethoxybenzaldehyde

To a stirred solution of 3-chloro-5-hydroxy benzaldehyde (0.5 g, 3.18 mmol) in DMF (5 mL, 10V) at room temperature, K₂CO₃ (1.3 g, 9.55 mmol) was added. The reaction mixture was stirred for 30 min, then 1-bromo ethane (0.353 g, 3.50 mmol) was added to the reaction mixture. Then the reaction mixture was stirred at room temperature for 4 h. The progress of the reaction was monitored by TLC analysis. After completion of the reaction, the reaction mixture was poured into ice cold water (80 mL). The solid precipitate was isolated by filtration, washed with cold water (2×20 mL) and dried under vacuum to provide 3-chloro-5-ethoxybenzaldehyde (0.5 g, 84.80% yield) as a white solid.

¹H NMR (400 MHz, d₆-DMSO) δ: 9.94 (s, 1H), 7.51 (s, 1H), 7.39 (s, 1H), 7.34 (d, J=2.0 Hz, 1H), 4.13 (q, J=7.2 Hz, 2H), 1.34 (t, J=7.2 Hz, 3H).

### Step-2: Synthesis of tert-butyl (S)-((1-(3-chloro-5-ethoxy benzyl) pyrrolidin-3-yl) methyl) carbamate

To a stirred solution of 3-chloro-5-ethoxy benzaldehyde (0.5 g, 2.70 mmol) in DCE (10 mL, 20V) at room temperature, tert-butyl (R)-(pyrrolidin-3-ylmethyl) carbamate hydrochloride (0.769 g, 3.24 mmol) was added. The reaction mixture was stirred at room temperature for 2 h. Sodium triacetoxyborohydride (1.7 g, 8.12 mmol) was added to the reaction mixture portion-wise at 0° C. The reaction mixture was stirred at room temperature for 16 h. The progress of the reaction was monitored by TLC and LCMS analysis. After completion of the reaction, the reaction mixture was poured into a saturated solution of sodium bicarbonate (70 mL) and extracted with CH₂Cl₂ (3×40 mL). The combined organic fractions were washed with water (50 mL), dried over sodium sulphate and concentrated under reduced pressure. The crude material was purified by normal phase column chromatography (4-5% MeOH in CH₂Cl₂) to provide tert-butyl (S)-((1-(3-chloro-5-ethoxybenzyl) pyrrolidin-3-yl) methyl) carbamate (0.6 g, 60.05% yield) as a yellowish sticky solid.

LCMS [ESI, M+1]: 369.0 (RT: 1.532 min, Purity: 100%),

Chiral HPLC: RT: 2.83 min, Purity: 100.00% ¹H NMR (400 MHz, d₆-DMSO) δ: 6.81-6.89 (m, 3H), 5.76 (s, 1H), 4.03 (q, J=6.8 Hz, 2H), 3.49 (d, J=7.9 Hz, 2H), 2.97-2.80-2.88 (m, 2H), 2.50-2.35 (m, 4H), 2.29-2.12 (m, 2H), 1.83-1.79 (m, 2H), 1.36 (t, J=7.0 Hz, 3H), 1.35 (s, 9H).

### Step-3: Synthesis of (S)-(1-(3-chloro-5-ethoxy benzyl) pyrrolidin-3-yl) methanamine hydrochloride (Compound 101)

To a stirred solution of tert-butyl (S)-((1-(3-chloro-5-ethoxy benzyl) pyrrolidin-3-yl) methyl) carbamate (0.6 g, 1.63 mmol) in CH₂Cl₂ (6 mL, 10V) at room temperature, 4M HCl in dioxane (3 mL, 5V) was added. The reaction mixture was stirred at room temperature for 2 h. The progress of the reaction was monitored by TLC and LCMS analysis. After completion of the reaction, the reaction mixture was concentrated under reduced pressure and the crude material purified by trituration using CH₂Cl₂ (2×30 mL) to provide (S)-(1-(3-chloro-5-ethoxy benzyl) pyrrolidin-3-yl) methanamine hydrochloride (0.4 g, 91.50%) as a white solid.

LCMS [ESI, M+1]: 269.0 (RT: 0.800 min, Purity: 99.70%),

HPLC: RT: 4.01 min, Purity: 99.81%

Chiral HPLC: RT: 2.88 min, Purity: 100.00%

Instrument Name: Shimadzu LC-20 AD

Chromatographic separation was conducted with Shimadzu LC-20 AD system with DAD detector. The column used was CHIRALPAK® IG (250×4 mm, 5 ␣m) and the compounds were eluted with, Mobile Phase A: Liq·CO2, Mobile Phase B: 0.1% Methanolic ammonia in Methanol:Acetonitrile (50:50) with Gradient method; with Flow rate=3 ml/min; analysis time 12 min.

¹H NMR (400 MHz, CD₃OD) δ: 7.08-7.21 (m, 3H), 4.41-4.35 (m, 2H), 4.09-4.14 (m, 2H), 3.62-3.72 (m, 2H), 3.48-3.53 (m, 2H), 3.10-3.20 (m, 2H), 3.08-2.70 (m, 1H), 2.30-2.50 (m, 1H), 1.80-2.10 (m, 1H), 1.42 (t, J=7.0 Hz, 3H).

### Experimental Protocol for (Compound 102)

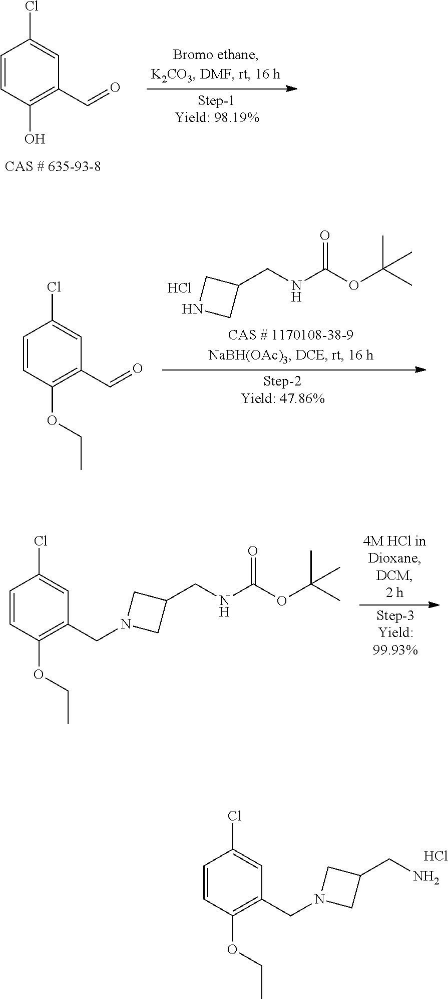

### Step-1: Synthesis of 5-chloro-2-ethoxybenzaldehyde

To a stirred solution of 5-chloro-2-hydroxybenzaldehyde (19 g, 12.18 mmol) in DMF (190 mL, 10V) at 0° C., Potassium carbonate (50.5 g, 36.54 mmol) was added. The reaction mixture was stirred at room temperature for 1 h. Then bromoethane (26.3 g, 24.35 mol) was added and reaction mixture was stirred at room temperature for 16 h. The progress of the reaction was monitored by TLC. After completion of the reaction, the reaction mixture was poured into ice cold water (100 mL). The solid precipitate was isolated by filtration and dried under reduced pressure to provide 5-chloro-2-ethoxybenzaldehyde (23 g, 98.19% yield) as off-white which was used directly in the next step of the synthesis.

### Step-2: Synthesis of tert-butyl ((1-(5-chloro-2-ethoxybenzyl) azetidin-3-yl) methyl) carbamate

To a stirred solution of 5-chloro-2-ethoxybenzaldehyde (0.50 g, 2.71 mmol) in DCE (10 mL, 20V) at room temperature, tert-butyl (azetidin-3-yl methyl) carbamate hydrochloride (0.60 g, 2.71 mmol) was added. The reaction mixture was stirred at room temperature for 2 h. Then sodium tri acetoxy borohydride (1.72 g, 8.15 mmol) was added portion wise to the reaction mixture at 0° C. The reaction was stirred at room temperature for 16 h. The progress of the reaction was monitored by TLC and LCMS analysis. After completion of the reaction, the reaction mixture was poured into a saturated solution of sodium bicarbonate (20 mL) and extracted with dichloromethane (3×20 mL). The combined organic fractions were washed with water (2×30 mL), dried over sodium sulphate evaporated and concentrated under reduced pressure. The crude material was purified by column chromatography (SiO₂; 30% ethyl acetate in hexane) to provide tert-butyl ((1-(5-chloro-2-ethoxybenzyl) azetidin-3-yl) methyl) carbamate (0.46 g, 47.88% yield) as a light-yellow liquid.

LCMS [ESI, (M, M+2)]: 355.0, 356.8 (RT: 1.416 min, Purity: 100%)

### Step-3: Synthesis of (1-(5-chloro-2-ethoxybenzyl) azetidin-3-yl) methanamine hydrochloride (Compound 102)

To a stirred solution of tert-butyl ((1-(5-chloro-2-ethoxy benzyl) azetidin-3-yl) methyl) carbamate (0.46 g, 1.29 mmol) in CH₂Cl₂ (4.6 mL, 10V) at 0° C., 4M HCl in dioxane (2.3 mL, 5V) was added. The reaction mixture was stirred at room temperature for 2 h. After completion of reaction, the reaction mixture was concentrated under reduced pressure. The crude material was diluted with water (10 mL) and washed with EtOAc (3×20 mL). The aqueous layer was lyophilized to provide (1-(5-chloro-2-ethoxy benzyl) azetidin-3-yl) methanamine hydrochloride (0.33 g, 99.93% yield) as a white solid.

LCMS [ESI, M, M+2]: 254.9, 256.7 (RT: 1.005 min, Purity: 100%),

HPLC: RT: 3.43 min, Purity: 95.51%, ¹H NMR (400 MHz, CD₃OD): δ 7.51 (d, J=2.5 Hz, 1H), 7.46 (dd, J=8.8, 2.6 Hz, 1H), 7.12 (d, J=8.9 Hz, 1H), 4.48-4.44 (m, 2H), 4.40-4.24 (m, 2H), 4.23-4.10 (m, 4H), 3.37-3.33 (m, 1H), 3.33-3.32 (m, 1H), 3.27-3.22 (m, 1H), 1.51 (t, J=7.0 Hz, 3H).

### Experimental Protocol for Salt Breaking

The salts of the compounds of the present invention are converted into the free base of the compounds of the present invention as follows.

10 V of MTBE and 7 V of water is added to 1 eq. of the hydrochloride salt of a compound of the present invention. The mixture is cooled to 8° C. at a rate of 0.3° C./min. Aqueous ammonia (NH₃·H₂O, 25%) is added to adjust the pH of the solution to 9-10. The mixture is stirred at 8° C. for 15 min. The organic phase is separated, and the aqueous phase is extracted twice with 8V of MTBE. The organic phases are combined and dried over anhydrous sodium sulfate. The solids are filtered off and washed three times with 2V of MTBE. The combined organic phases are concentrated under vacuum at room temperature and subsequently the oil is vacuum dried at 40° C. for 3 h.

### Abbreviations

TLC Thin Layer Chromatography
LCMS Liquid chromatography-mass spectrometry
DMF Dimethylformamide
DMSO Dimethylsulfoxide
DCE Dichloroethane
DCM Dichloromethane
KTB Potassium tert-butoxide (KOtBu)
MTBE Methyl tert-butyl ether

TABLE 1
Compounds 1 to 18, 19-56, 57, 58, 60, 61, 63, 64, 66, 67, 69, and 70-102 of the
present invention with chemical name

| Compound | | |
|---|---|---|
| No. | Structure | Chemical name |
| 1 | 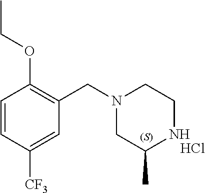 | (S)-1-(2-ethoxy-5- (trifluoromethyl)benzyl)-3- methylpiperazine hydrochloride |
| 2 | 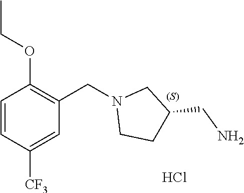 | (S)-(1-(2-ethoxy-5-(trifluoromethyl) benzyl) pyrrolidin-3-yl) methanamine hydrochloride |
| 3 | 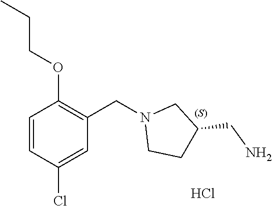 | (S)-(1-(5-chloro-2-propoxybenzyl) pyrrolidin-3-yl) methanamine hydrochloride |
| 4 | 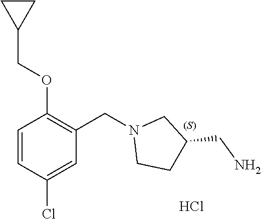 | (S)-(1-(5-chloro-2- (cyclopropylmethoxy)benzyl)pyrrolidin-3- yl)methanamine hydrochloride |
| 5 | 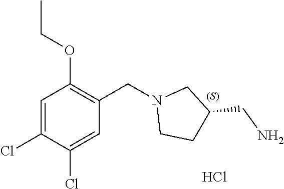 | (S)-(1-(4,5-dichloro-2- ethoxybenzyl)pyrrolidin-3- yl)methanamine hydrochloride |
| 6 | 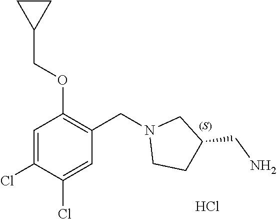 | (S)-(1-(4,5-dichloro-2- (cyclopropylmethoxy)benzyl)pyrrolidin-3- yl)methanamine hydrochloride |
| 7 | 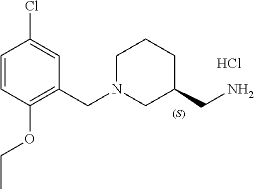 | (S)-(1-(5-chloro-2- ethoxybenzyl) piperidin-3- yl)methanamine hydrochloride |
| 8 | 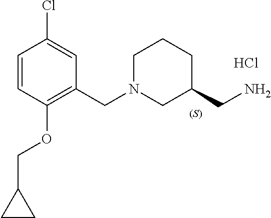 | (S)-(1-(5-chloro-2- (cyclopropylmethoxy)benzyl)piperidin-3- yl)methanamine hydrochloride |
| 9 | 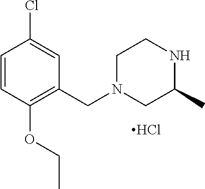 [SMILES: CCOc1ccc(Cl)cc1CN1CCN[C@@H](C)C1 ; Cl] | (S)-1-(5-chloro-2-ethoxybenzyl)-3- methylpiperazine hydrochloride |
| 10 | 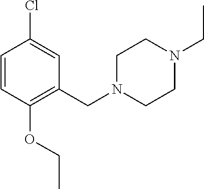 [SMILES: CCOc1ccc(Cl)cc1CN1CCN(CC)CC1] | 1-(5-chloro-2-ethoxybenzyl)-4- ethylpiperazine |
| 11 | 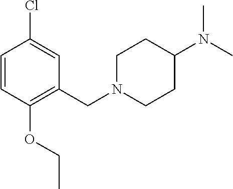 [SMILES: CCOc1ccc(Cl)cc1CN1CCC(N(C)C)CC1] | 1-(5-chloro-2-ethoxybenzyl)-N,N- dimethylpiperidin-4-amine |
| 12 | 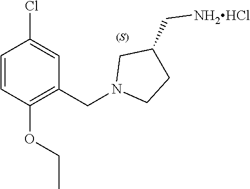 | (S)-(1-(5-chloro-2- ethoxybenzyl)pyrrolidin-3- yl)methanamine hydrochloride |
| 13 | 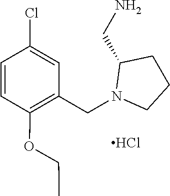 [SMILES: CCOc1ccc(Cl)cc1CN1CCC[C@H]1CN ; Cl] | (S)-(1-(5-chloro-2- ethoxybenzyl)pyrrolidin-2- yl)methanamine hydrochloride |
| 14 | 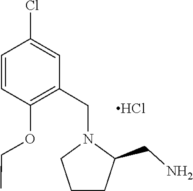 [SMILES: CCOc1ccc(Cl)cc1CN1CCC[C@@H]1CN ; Cl] | (R)-(1-(5-chloro-2- ethoxybenzyl) pyrrolidin-2- yl)methanamine hydrochloride |
| 15 | 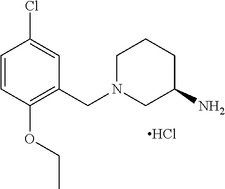 [SMILES: CCOc1ccc(Cl)cc1CN1CCC[C@@H](N)C1 ; Cl] | (R)-1-(5-chloro-2-ethoxybenzyl)piperidin- 3-amine hydrochloride |
| 16 | 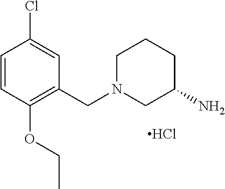 [SMILES: CCOc1ccc(Cl)cc1CN1CCC[C@H](N)C1 ; Cl] | (S)-1-(5-chloro-2-ethoxybenzyl)piperidin- 3-amine hydrochloride |
| 17 | 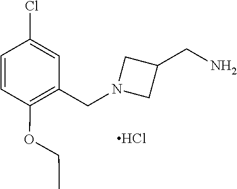 [SMILES: CCOc1ccc(Cl)cc1CN1CC(CN)C1 ; Cl] | (1-(5-chloro-2-ethoxybenzyl)azetidin-3- yl)methanamine hydrochloride |
| 18 | 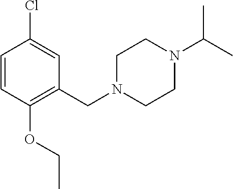 [SMILES: CCOc1ccc(Cl)cc1CN1CCN(C(C)C)CC1] | 1-(5-chloro-2-ethoxybenzyl)-4- isopropylpiperazine |
| 19 | 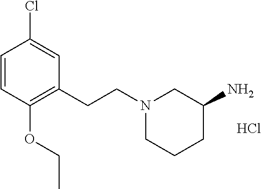 [SMILES: CCOc1ccc(Cl)cc1CCN1CCC[C@H](N)C1 ; Cl] | (S)-1-(5-chloro-2- ethoxyphenethyl)piperidin-3-amine hydrochloride |
| 20 | 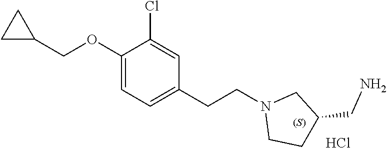 | (S)-(1-(3-chloro-4- (cyclopropylmethoxy)phenethyl)pyrrolidin- 3-yl)methanamine hydrochloride |
| 21 | 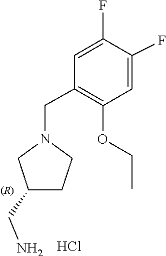 | (R)-(1-(2-ethoxy-4,5- difluorobenzyl)pyrrolidin-3- yl)methanamine hydrochloride |
| 22 | 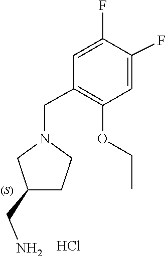 | (S)-(1-(2-ethoxy-4,5- difluorobenzyl)pyrrolidin-3- yl)methanamine hydrochloride |
| 23 | 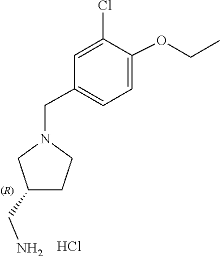 | (R)-(1-(3-chloro-4- ethoxybenzyl)pyrrolidin-3-yl) methanamine hydro chloride |
| 24 | 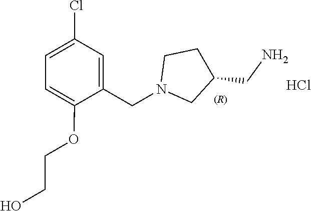 | (R)-2-(2-((3-(aminomethyl)pyrrolidin-1- yl)methyl)-4-chlorophenoxy)ethan-1-ol hydrochloride |
| 25 | 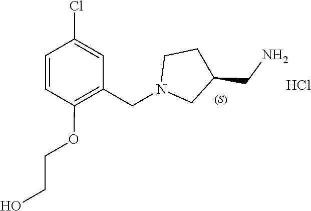 | (S)-2-(2-((3-(aminomethyl)pyrrolidin-1- yl)methyl)-4-chlorophenoxy)ethan-1-ol hydrochloride |
| 26 | 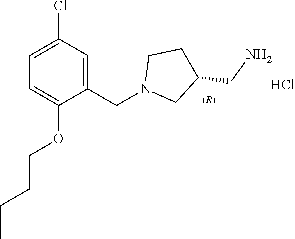 | (R)-(1-(2-butoxy-5- chlorobenzyl)pyrrolidin-3-yl)methanamine hydrochloride |
| 27 | 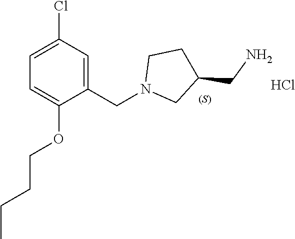 | (S)-(1-(2-butoxy-5- chlorobenzyl)pyrrolidin-3-yl)methanamine hydrochloride |
| 28 | 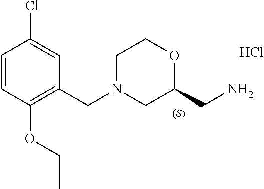 | (S)-(4-(5-chloro-2- ethoxybenzyl)morpholin-2- yl)methanamine hydrochloride |
| 29 | 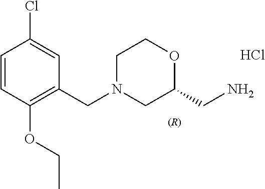 | (R)-(4-(5-chloro-2- ethoxybenzyl)morpholin-2- yl)methanamine hydrochloride |
| 30 | 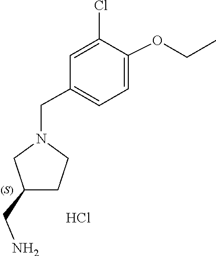 | (S)-(1-(3-chloro-4- ethoxybenzyl)pyrrolidin-3- yl)methanamine hydrochloride |
| 31 | 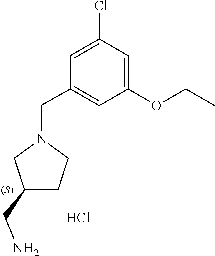 | (S)-(1-(3-chloro-5- ethoxybenzyl)pyrrolidin-3- yl)methanamine hydro chloride |
| 32 | 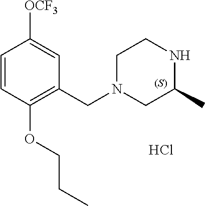 | (S)-3-methyl-1-(2-propoxy-5- (trifluoromethoxy)benzyl) piperazine hydrochloride |
| 33 | 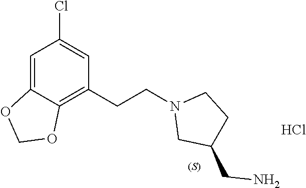 | (S)-(1-(2-(6-chlorobenzo[d][1,3]dioxol-4- yl)ethyl)pyrrolidin-3-yl)methanamine hydrochloride |
| 34 | 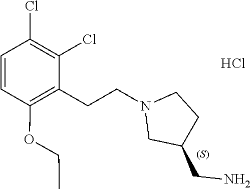 | (S)-(1-(2,3-dichloro-6- ethoxyphenethyl)pyrrolidin-3- yl)methanamine hydrochloride |
| 35 | 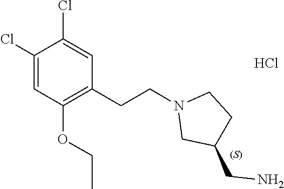 | (S)-(1-(4,5-dichloro-2- ethoxyphenethyl)pyrrolidin-3- yl)methanamine hydrochloride |
| 36 | 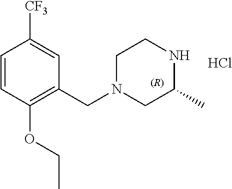 | (R)-1-(2-ethoxy-5-(trifluoromethyl)benzyl)- 3-methylpiperazine hydrochloride |
| 37 | 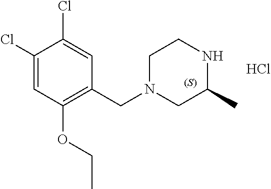 | (S)-1-(4,5-dichloro-2-ethoxybenzyl)-3- methylpiperazine hydrochloride |
| 38 | 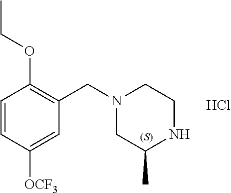 | (S)-1-(2-ethoxy-5- (trifluoromethoxy)benzyl)-3- methylpiperazine hydrochloride |
| 39 | 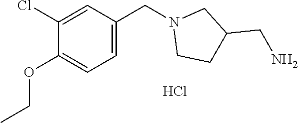 [SMILES: CCOc1ccc(CN2CCC(CN)C2)cc1Cl ; Cl] | (1-(3-chloro-4-ethoxybenzyl)pyrrolidin-3- yl)methanamine hydrochloride |
| 40 | 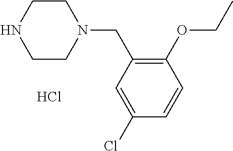 [SMILES: CCOc1ccc(Cl)cc1CN1CCNCC1 ; Cl] | 1-(5-chloro-2-ethoxybenzyl)piperazine hydrochloride |
| 41 | 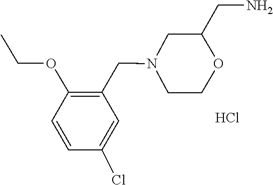 [SMILES: CCOc1ccc(Cl)cc1CN1CCOC(CN)C1 ; Cl] | (4-(5-chloro-2-ethoxybenzyl)morpholin-2- yl)methanamine hydrochloride |
| 42 | 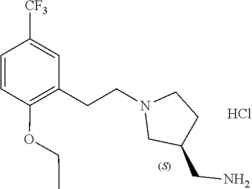 | (S)-(1-(2-ethoxy-5- (trifluoromethyl)phenethyl)pyrrolidin-3- yl)methanamine hydrochloride |
| 43 | 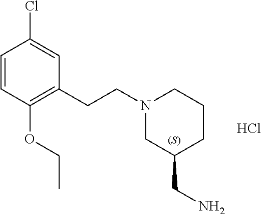 | (S)-(1-(5-chloro-2- propoxyphenethyl)piperidin-3- yl) methanamine hydrochloride |
| 44 | 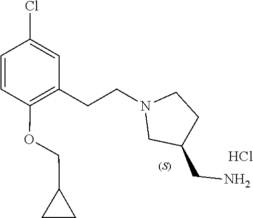 | (S)-(1-(5-chloro-2- (cyclopropylmethoxy)phenethyl)pyrrolidin- 3-yl)methanamine hydrochloride |
| 45 | 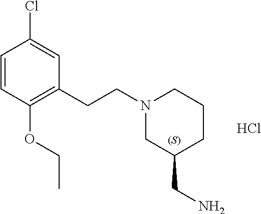 | (S)-(1-(5-chloro-2- ethoxyphenethyl)piperidin-3- yl)methanamine hydrochloride |
| 46 | 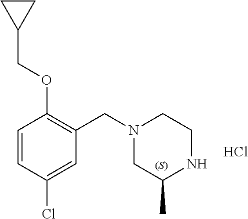 | (S)-1-(5-chloro-2- (cyclopropylmethoxy)benzyl)-3- methylpiperazine hydrochloride |
| 47 | 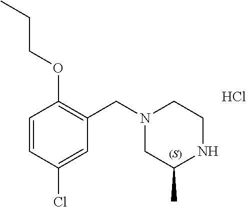 | (S)-1-(5-chloro-2-propoxybenzyl)-3- methylpiperazine hydrochloride |
| 48 | 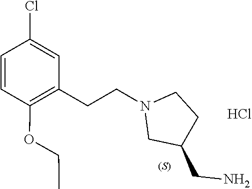 | (S)-(1-(5-chloro-2- propoxyphenethyl)pyrrolidin-3- yl)methanamine hydrochloride |
| 49 | 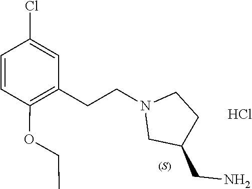 | (S)-(1-(5-chloro-2- ethoxyphenethyl)pyrrolidin-3- yl)methanamine hydrochloride |
| 50 | 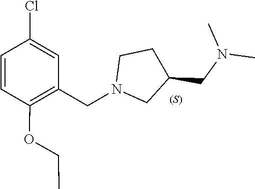 | (S)-1-(1-(5-chloro-2- ethoxybenzyl)pyrrolidin-3-yl)-N,N- dimethyl methanamine |
| 51 | 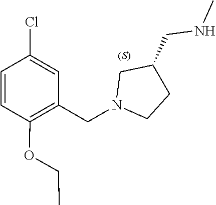 | (S)-1-(1-(5-chloro-2- ethoxybenzyl)pyrrolidin-3-yl)-N- methylmethanamine |
| 52 | 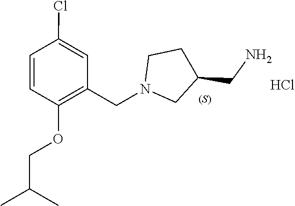 | (S)-(1-(5-chloro-2- isobutoxybenzyl)pyrrolidin-3- yl)methanamine hydrochloride |
| 53 | 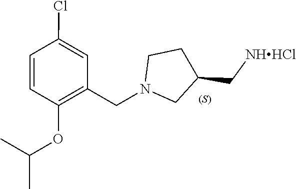 | (S)-(1-(5-chloro-2- isopropoxybenzyl)pyrrolidin-3- yl)methanamine hydrochloride |
| 54 | 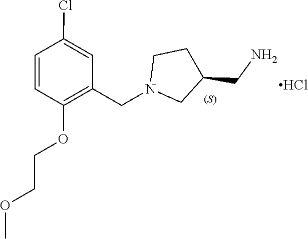 | (S)-(1-(5-chloro-2-(2- methoxyethoxy)benzyl)pyrrolidin-3- yl)methanamine hydrochloride |
| 55 | 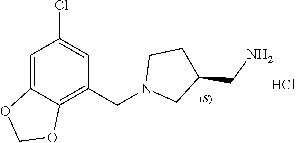 | (S)-(1-((6-chlorobenzo[d][1,3]dioxol-4- yl)methyl)pyrrolidin-3-yl)methanamine hydrochloride |
| 56 | 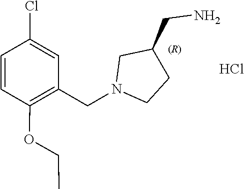 | (R)-(1-(5-chloro-2- ethoxybenzyl)pyrrolidin-3- yl)methanamine hydrochloride |
| 57 | 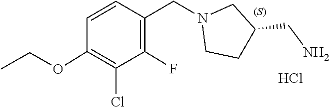 | (S)-(1-(3-chloro-4-ethoxy-2- fluorobenzyl)pyrrolidin-3-yl)methanamine hydrochloride |
| 58 | 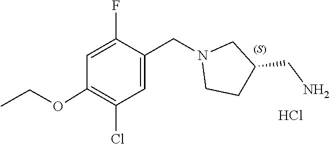 | (S)-(1-(5-chloro-4-ethoxy-2- fluorobenzyl)pyrrolidin-3-yl) methanamine hydrochloride |
| 60 | 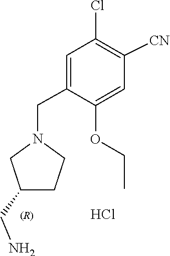 | (R)-4-((3-(aminomethyl)pyrrolidin-1- yl)methyl)-2-chloro-5-ethoxy benzonitrile hydrochloride |
| 61 | 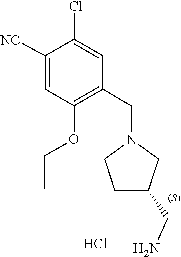 | (S)-4-((3-(aminomethyl)pyrrolidin-1- yl)methyl)-2-chloro-5-ethoxybenzonitrile hydrochloride |
| 63 | 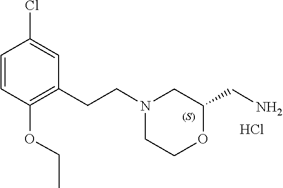 | (S)-(4-(5-chloro-2- ethoxyphenethyl)morpholin-2- yl)methanamine hydrochloride |
| 64 | 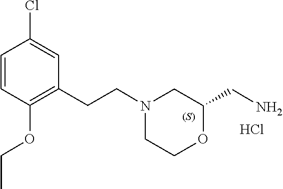 | (S)-3-(2-(3-(aminomethyl)piperidin-1- yl)ethyl)-4-ethoxybenzonitrile hydrochloride |
| 66 | 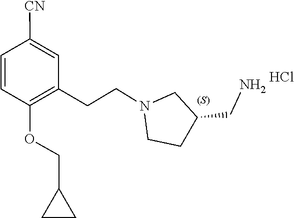 | (S)-3-(2-(3-(aminomethyl)pyrrolidin-1- yl)ethyl)-4- (cyclopropylmethoxy)benzonitrile hydrochloride |
| 67 | 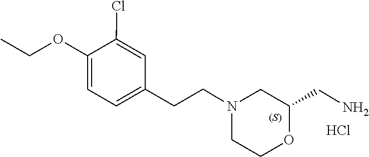 | (S)-(1-(3-chloro-4- ethoxyphenethyl)piperidin-3- yl)methanamine hydrochloride |
| 69 | 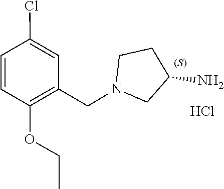 | (S)-1-(5-chloro-2-ethoxybenzyl)pyrrolidin- 3-amine hydrochloride |
| 70 | 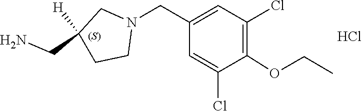 | (S)-(1-(3,5-dichloro-4- ethoxybenzyl)pyrrolidin-3- yl)methanamine hydrochloride |
| 71 | 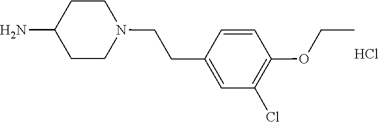 [SMILES: CCOc1ccc(CCN2CCC(N)CC2)cc1Cl ; Cl] | 1-[2-(3-chloro-4-ethoxyphenyl)ethyl]-4- piperidylamine hydrochloride |
| 72 | 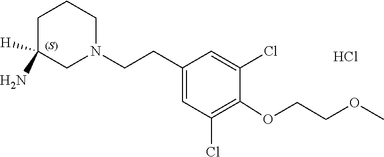 | (S)-1-{2-[3,5-dichloro-4-(2- methoxyethoxy)phenyl]ethyl}-3- piperidylamine hydrochloride |
| 73 | 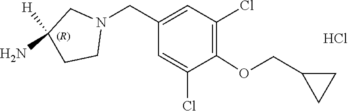 | (R)-1-{[3,5-dichloro-4- (cyclopropylmethoxy)phenyl]methyl}-3- pyrrolidinylamine hydrochloride |
| 74 | 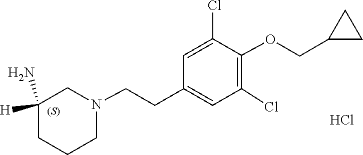 | (S)-1-{2-[3,5-dichloro-4- (cyclopropylmethoxy)phenyl]ethyl}-3- piperidylamine hydrochloride |
| 75 | 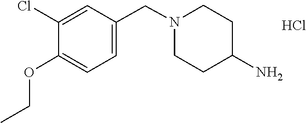 [SMILES: CCOc1ccc(CN2CCC(N)CC2)cc1Cl ; Cl] | 1-[(3-chloro-4-ethoxyphenyl)methyl]-4- piperidylamine hydrochloride |
| 76 | 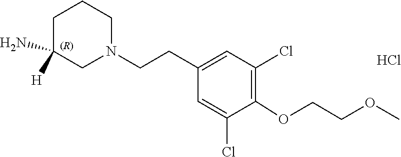 | (R)-1-{2-[3,5-dichloro-4-(2- methoxyethoxy)phenyl]ethyl}-3- piperidylamine hydrochloride |
| 77 | 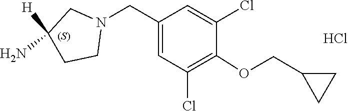 | (S)-1-{[3,5-dichloro-4- (cyclopropylmethoxy)phenyl]methyl}-3- pyrrolidinylamine hydrochloride |
| 78 | 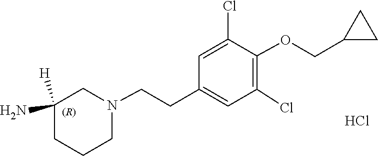 | (R)-1-{2-[3,5-dichloro-4- (cyclopropylmethoxy)phenyl]ethyl}-3- piperidylamine hydrochloride |
| 79 | 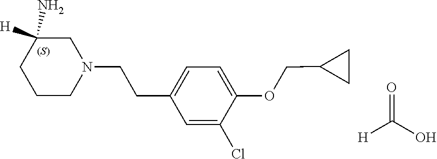 | (S)-1-{2-[3-chloro-4- (cyclopropylmethoxy)phenyl]ethyl}-3- piperidylamine formate |
| 80 | 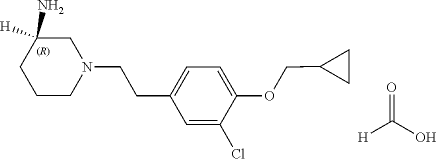 | (R)-1-{2-[3-chloro-4- (cyclopropylmethoxy)phenyl]ethyl}-3- piperidylamine formate |
| 81 | 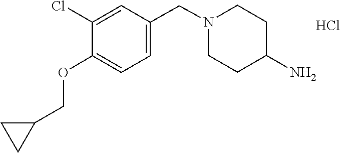 [SMILES: NC1CCN(Cc2ccc(OCC3CC3)c(Cl)c2)CC1 ; Cl] | 1-{[3-chloro-4- (cyclopropylmethoxy)phenyl]methyl}-4- piperidylamine hydrochloride |
| 82 | 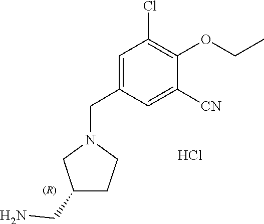 | 5-{[(R)-3-(Aminomethyl)-1- pyrrolidinyl]methyl}-3-chloro-2- ethoxybenzonitrile hydrochloride |
| 83 | 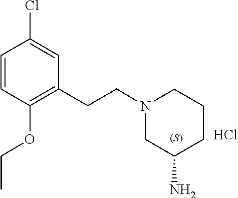 | 3-{2-[(S)-3-Amino-1-piperidyl]ethyl}-4- ethoxybenzonitrile hydrochloride |
| 84 | 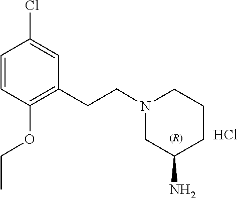 | 3-{2-[(R)-3-Amino-1-piperidyl]ethyl}-4- ethoxybenzonitrile hydrochloride |
| 85 | 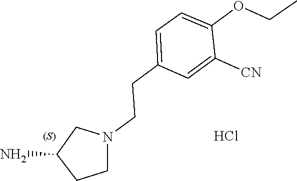 | 5-{2-[(S)-3-amino-1-pyrrolidinyl]ethyl}-2- ethoxybenzonitrile hydrochloride |
| 86 | 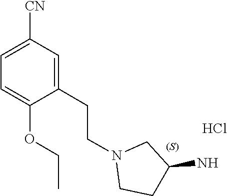 | 3-{2-[(S)-3-Amino-1-pyrrolidinyl]ethyl}-4- ethoxybenzonitrile hydrochloride |
| 87 | 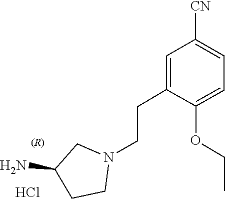 | 3-{2-[(R)-3-amino-1-pyrrolidinyl]ethyl}-4- ethoxybenzonitrile hydrochloride |
| 88 | 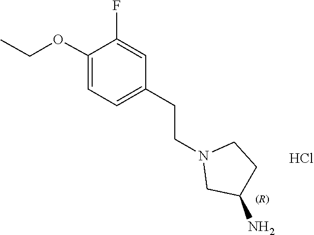 | (R)-1-[2-(4-ethoxy-3-fluorophenyl)ethyl]- 3-pyrrolidinylamine hydrochloride |
| 89 | 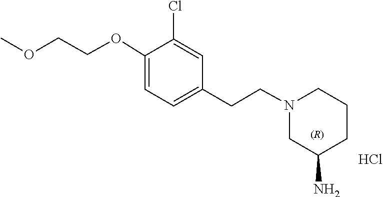 | (R)-1-{2-[3-chloro-4-(2- methoxyethoxy)phenyl]ethyl}-3- piperidylamine hydrochloride |
| 90 | 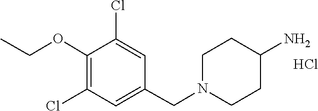 [SMILES: CCOc1c(Cl)cc(CN2CCC(N)CC2)cc1Cl ; Cl] | 1-[(3,5-Dichloro-4-ethoxyphenyl)methyl]- 4-piperidylamine hydrochloride |
| 91 | 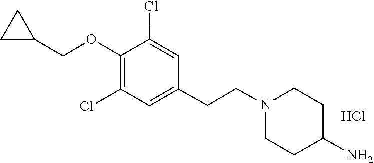 [SMILES: NC1CCN(CCc2cc(Cl)c(OCC3CC3)c(Cl)c2)CC1 ; Cl] | 1-{2-[3,5-Dichloro-4- (cyclopropylmethoxy)phenyl]ethyl}-4- piperidylamine hydrochloride |
| 92 | 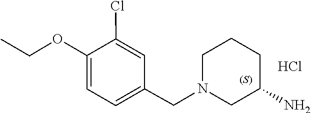 | (S)-1-[(3-chloro-4-ethoxyphenyl)methyl]- 3-piperidylamine hydrochloride |
| 93 | 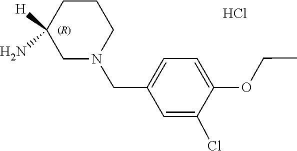 | (R)-1-[(3-chloro-4-ethoxyphenyl)methyl]- 3-piperidylamine hydrochloride |
| 94 | 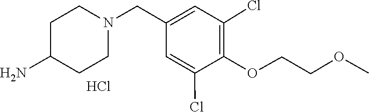 [SMILES: COCCOc1c(Cl)cc(CN2CCC(N)CC2)cc1Cl ; Cl] | 1-{[3,5-dichloro-4-(2- methoxyethoxy)phenyl]methyl}-4- piperidylamine hydrochloride |
| 95 | 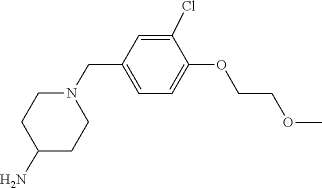 [SMILES: COCCOc1ccc(CN2CCC(N)CC2)cc1Cl] | 1-{[3-chloro-4-(2- methoxyethoxy)phenyl]methyl}-4- piperidylamine hydrochloride |
| 96 | 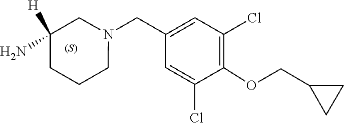 | (S)-1-{[3,5-dichloro-4- (cyclopropylmethoxy)phenyl]methyl}-3- piperidylamine hydrochloride |
| 97 | 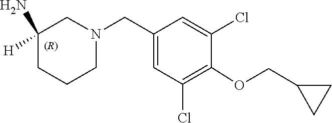 | (R)-1-{[3,5-dichloro-4- (cyclopropylmethoxy)phenyl]methyl}-3- piperidylamine hydrochloride |
| 98 | 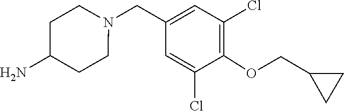 [SMILES: NC1CCN(Cc2cc(Cl)c(OCC3CC3)c(Cl)c2)CC1] | 1-{[3,5-dichloro-4- (cyclopropylmethoxy)phenyl]methyl}-4- piperidylamine hydrochloride |
| 99 | 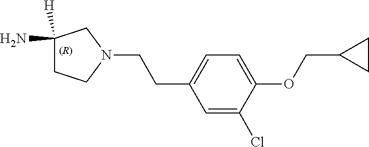 | (R)-1-{2-[3-chloro-4- (cyclopropylmethoxy)phenyl]ethyl}-3- pyrrolidinylamine hydrochloride |
| 100 | 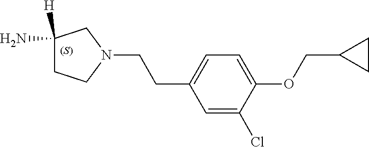 | (S)-1-{2-[3-chloro-4- (cyclopropylmethoxy)phenyl]ethyl}-3- pyrrolidinylamine hydrochloride |
| 101 | 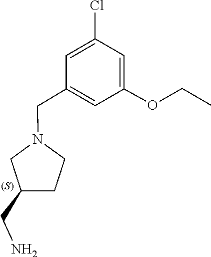 | ({(S)-1-[(3-chloro-5-ethoxyphenyl)methyl]- 3-pyrrolidinyl}methyl)amine hydrochloride |
| 102 | 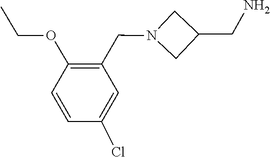 [SMILES: CCOc1ccc(Cl)cc1CN1CC(CN)C1] | ({1-[(5-chloro-2-ethoxyphenyl)methyl]-3- azetidinyl}methyl)amine hydrochloride |

### Biological Assays and Data

As stated above, the compounds of the present invention induce and/or stimulate autophagy and are useful in treating autophagy-related diseases. The biological activity of the compounds of the present invention can be determined by any appropriate test to determine the ability to induce and/or stimulate autophagy.

### Assessment of Stimulation of Autophagy

Lysosomes play a fundamental role in the autophagic pathway by fusing with autophagosomes and creating ‘autolysosomes’ in order to digest their contents. Stimulation of lysosome and autolysosome formation by a compound is indicative of a stimulation of autophagy. The ability of compounds to stimulate lysosome and autolysosome formation, and thus autophagy, in live cells was assessed via fluorescent microscopy using various fluorescent stains for labelling and tracking acidic organelles (including lysosomes and autolysosomes) such as: LysoView™ 650 (70059 and 70059-T, Biotium), LysoView™633 (70058 and 70058-T, Biotium) and LysoTracker™ Deep Red (L12492, ThermoFisher Scientific). The cellular phenotype was quantitatively assessed for the induction of acidic vesicle formation and compared to a non-treated control, thus providing a measure of the ability of the compound under investigation to stimulate autophagy.

Representative procedure using LysoView™633 dye or LysoTracker™ Deep Red dye: Human osteosarcoma U2OS cells (40,000 cells/well) were seeded in a 24 well glass bottom plate (Sensoplate, Greiner Bio-One) and were incubated overnight in a humidified atmosphere at 37° C. and 5% CO2. Cells were grown in DMEM (Gibco) supplemented with 10% Fetal Bovine Serum (FBS) and 100 units/ml penicillin and 100 μg/ml streptomycin (Invitrogen). After the attachment period, cells were treated with different compounds of interest (at various concentrations in DMSO) or DMSO (non-treated control) in cell culture medium and were incubated for 24 hours. Compound-containing medium was removed and cells were incubated with pre-warmed cell culture medium containing 1× LysoView™ 633 (70058 and 70058-T, Biotium) or 50 nM LysoTracker™ Deep Red (L12492, ThermoFisher Scientific) for 45 minutes at 37° C. Finally, cell nuclei were stained for 10 minutes using Hoechst 33342 (1 μg/mL) and then the medium was replaced with fresh medium. The 24 well plate was then fitted into a heated stage on the microscope and cells maintained at 37° C. Images were captured using appropriate filter set for Cy5 and DAPI detection with the EVOS M7000 Microscope (ThermoFisher Scientific). The cellular phenotype was visually assessed for the induction of acidic vesicle formation relative to the non-treated control; multiple images were acquired and analysed using ImageJ or Cell Profiler.

Compounds 1-18, 19-56, 57, 58, 60, 61, 63, 64, 66, 67 and 69 showed increased acidic vesicle formation in human osteosarcoma U20S cells relative to a DMSO-treated control (non-treated control) at a concentration of 10 μM. In this assay, acidic vesicle formation was increased by at least 25% for compounds 1-18. Compounds 19-56, 57, 58, 60, 61, 63, 64, 66, 67 and 69 also showed an increase in acidic vesicle formation in this assay by at least 25% over the DMSO-treated control. This is evidence for significant stimulation of autophagy by these compounds under these conditions.

Selected compounds of this invention were also found to show increased acidic vesicle formation in human osteosarcoma U20S cells relative to a DMSO-treated control (non-treated control) at a concentration of 0.8 μM. This is evidence for significant stimulation of autophagy by these compounds under these conditions. Table 2 shows this data for selected compounds. Compounds having an activity designated as “+” provided a percentage increase in acidic vesicle formation of between 10% and 30% over the DMSO-treated control. Compounds having an activity designated as “++” provided a percentage increase in acidic vesicle formation of between 30% and 50% over the DMSO-treated control. Compounds having an activity designated as “+++” provided a percentage increase in acidic vesicle formation of between 50% and 100% over the DMSO-treated control. Compounds having an activity designated as “++++” provided a percentage increase in acidic vesicle formation of above 100% over the DMSO-treated control.

TABLE 2

| Compound No. | Activity |
|---|---|
| 2 | + |
| 3 | ++ |
| 4 | ++ |
| 5 | ++ |
| 6 | +++ |
| 7 | ++ |
| 8 | + |
| 9 | + |
| 10 | + |
| 11 | + |
| 12 | ++ |
| 15 | + |
| 17 | +++ |
| 18 | + |
| 25 | ++ |
| 26 | ++++ |
| 27 | ++++ |
| 28 | + |
| 29 | +++ |
| 30 | ++++ |
| 31 | ++++ |
| 32 | +++ |
| 36 | + |
| 37 | + |
| 38 | + |
| 39 | ++ |
| 40 | + |
| 41 | + |
| 42 | +++ |
| 43 | +++ |
| 44 | +++ |
| 45 | +++ |
| 46 | + |
| 47 | ++ |
| 48 | + |
| 50 | + |
| 52 | + |
| 55 | ++++ |
| 56 | ++++ |

Assessment of stimulation of autophagy—Tandem reporter assay (to assess autophagic flux)

Selected compounds of this invention were also assessed for their ability to stimulate autophagy using a U2OS cell line stabling expressing RFP-eGFP-hLC3b (a tandem reporter cell line). This assay is further described in WO 2023/089074.

During the process of autophagy, the cargo to be degraded is first enveloped by organelles called autophagosomes. These autophagosomes then fuse with lysosomes, causing them to become acidified and their digestive enzymes to become activated. The term “autophagic flux” is used to represent the dynamic process of autophagy: autophagic flux refers to the whole process of autophagy, including autophagosome formation, maturation, fusion with lysosomes, subsequent breakdown and the release of macromolecules back into the cytosol (Zhang X J, Chen S, Huang K X, Le W D. Why should autophagic flux be assessed? Acta Pharmacol Sin. 2013 May; 34(5): 595-9. doi: 10.1038/aps.2012.184. Epub 2013 Mar. 11). Compounds that stimulate autophagic flux stimulate the dynamic process of autophagy (the whole process of autophagy).

LC3b is a protein found in the membrane of autophagosomes which has been used to generate genetic reporters of autophagy in cells. In these systems a tandem fusion of LC3b is engineered with two fluorescent proteins of different wavelengths: one which is acid sensitive (for example eGFP, which fluoresces green) and the other which is acid insensitive (for example RFP, which fluoresces red). Cells expressing RFP-eGFP-hLC3b can be examined using fluorescent microscopy: the autophagosomes present will fluoresce in both channels (either yellow in an overlay of both channels or puncta that are present in both the individual red and green channels) but the autolysosomes present will fluoresce in the RFP (red) channel only. Using a fluorescent microscope, the number of autophagosomes and autolysosomes can be counted, allowing for the monitoring of both the induction of autophagy (total puncta count) and the rate of autophagic flux (ratio of red-only to red-and-green puncta). The influence of small molecules on autophagy induction and flux can be assessed using this system by comparison to a non-treated control.

Representative Procedure: Human osteosarcoma U20S cells stably expressing RFP-eGFP-hLC3b were seeded (10,000 cells/well) into a 96-well glass-bottom plate (Cell Carrier Ultra, Perkin Elmer) in DMEM Glutamax media (Gibco) supplemented with 10% Fetal Bovine Serum (FBS) and antibiotics (100 μg/ml penicillin, 100 μg/ml streptomycin, Invitrogen), then incubated overnight in a humified atmosphere at 37° C. and 5% CO₂. The cells were then treated with the compounds of interest, dissolved in DMSO, in a duplicated 5- or 10-point dose-response. 8 wells were treated with an equivalent volume of DMSO, 4 with 0.3 μM torin-1 as a positive control (MCE) and 4 wells with 0.4 μM torin-1 plus 0.2 μM bafilomycin (MCE) as a control for flux inhibition. The cells were returned to the incubator and the treatment continued for 24 hours. After treatment, the media was aspirated off and the cells fixed with a solution of 4% formaldehyde+1% glutaraldehyde (Sigma) in dPBS (plus magnesium and calcium, Gibco). Fixation was allowed to proceed for 15 minutes at room temperature then the fixative solution discarded and replaced with PBS (Gibco) plus Hoescht 33342 (1 μg/ml, Sigma). After 30 minutes the plates were imaged on an Opera Phenix confocal microscope (Perkin-Elmer) using a 40× water objective, collecting using DAPI, mCherry and GFP channels. Cells were detected based upon the staining of their nuclei with DAPI using the Harmony image analysis software (Perkin-Elmer) and the number of autophagosomes (GFP and RFP puncta) and autolysosomes (RFP only puncta) per cell were counted using the spot picking function within the software.

Selected compounds of this invention showed an increase in the number of RFP-only puncta (autolysosomes) relative to a DMSO-treated control (non-treated control) at different concentrations in a U20S cell line stabling expressing RFP-eGFP-hLC3b (a tandem reporter cell line). This is evidence for significant stimulation of autophagic flux (and thus the whole process of autophagy) by the compounds of this invention, and particularly by these compounds, under the conditions as tested. Table 3 shows this data for selected compounds. “Autolysosome % DMSO activity” is the activity of the compound (based on the number of RFP-only puncta ((autolysosomes)) as a percentage of the activity of the DMSO-treated control (non-treated control). Compounds having an activity designated as “+” provided a “Autolysosome % DMSO activity” value of between 150% and 200%. Compounds having an activity designated as “++” provided a “Autolysosome % DMSO activity” value of between 200% and 250%. Compounds having an activity designated as “+++” provided a “Autolysosome % DMSO activity” value of between 250% and 300%. Compounds having an activity designated as “++++” provided a “Autolysosome % DMSO activity” value of over 300%. Classification is based on the average of two runs unless otherwise stated.

TABLE 3

| Compound No. | Concentration (μM) | Activity |
|---|---|---|
| 74 | 1.25 | ++++ |
| 78 | 1.25 | ++ |
| 72 | 0.5 | + |
| 76 | 0.5 | + |
| 75 | 1.25 | ++ |
| 73 | 1.25 | +++ |
| 77 | 1.25 | +++ |
| 79 | 1.25 | +++ |
| 80 | 1.25 | +++ |
| 81 | 1.25 | + |
| 71 | 0.833 | + |
| 70 | 0.75 | + (one run) |
| 82 | 1.25 | + (one run) |
| 83 | 1.25 | ++ |
| 84 | 1.25 | ++ |
| 85 | 1.25 | + (one run) |
| 86 | 1.25 | + |
| 87 | 1.25 | + |
| 88 | 1.25 | + |
| 89 | 1.25 | + |
| 90 | 1.25 | ++ |
| 91 | 1.25 | + (one run) |
| 92 | 0.5 | + |
| 93 | 1.25 | + |
| 94 | 1.25 | + |
| 95 | 1.25 | ++ |
| 96 | 1.25 | ++ |
| 97 | 1.25 | + |
| 98 | 1.25 | + |
| 99 | 1.25 | + |
| 100 | 1.25 | + |

### Demonstration of Activity in a Cellular Model of Parkinson's Disease (PD)

Compound 12 was selected as an example compound of this invention to be evaluated in this model.

This model (supplied by Neuro-Sys SAS, France) is based on a primary culture of dopaminergic Tyrosine hydroxylase (TH)-positive neurons injured with the dopaminergic neurons-specific toxin (DA-toxin) 1-methyl-4-phenyl-1,2,3,6 tetrahydropyridine (MPTP) (Dauer and Przedborski, 2003). Any substances reducing DA-toxin neurotoxicity may be useful as a new therapeutic agent for the treatment or prevention of PD.

### Methods

Primary culture of mesencephalic neurons: Rat dopaminergic neurons were cultured as described by Visanji et al., 2008 and Callizot et al., 2019. Briefly, pregnant female rat (Wistar) of 15 days of gestation were killed using a deep anesthesia with CO2 chamber and a cervical dislocation. The midbrains obtained from 15-day-old rat embryos (Janvier, France) were dissected under a microscope. The embryonic midbrains will be removed and placed in ice-cold medium of Leibovitz (L15) containing 2% of Penicillin-Streptomycin (PS) and 1% of bovine serum albumin (BSA). The ventral portion of the mesencephalic flexure, a region of the developing brain rich in dopaminergic neurons, was used for the cell preparations.

The midbrains were dissociated by trypsinisation for 20 min at 37° C. (solution at a final concentration of 0.05% trypsin and 0.02% EDTA). The reaction was stopped by the addition of Dulbecco's modified Eagle's medium (DMEM) containing DNAase I grade II (0.5 mg/mL) and 10% of foetal calf serum (FCS). Cells were then mechanically dissociated by 3 passages through a 10 ml pipette. Cells were then centrifuged at 180×g for 10 min at +4° C. on a layer of BSA (3.5%) in L15 medium. The supernatant was discarded and the cell pellets re-suspended in a defined culture medium consisting of Neurobasal supplemented with B27 (2%), L-glutamine (2 mM) and 2% of PS solution and 10 ng/mL of Brain-derived neurotrophic factor (BDNF) and 1 ng/mL of Glial-Derived Neurotrophic Factor (GDNF). Viable cells were counted in a Neubauer cytometer using the trypan blue exclusion test. The cells were seeded at a density of 40,000 cells/well in 96 well-plates (pre-coated with poly-L-lysine) and maintained in a humidified incubator at 37° C. in 5% CO2/95% air atmosphere. Half of the medium was changed every 2 days with fresh medium. The wells of first and last lines and columns were used (to avoid any edge effect) and were filled with sterile water.

Test compound and MPP+:
(i) Pre-incubation. On day 6 of culture, the test compound was dissolved in PBS or DMSO and incubated for 1 hour before the MPP+ exposure.
(ii) Injury. One hour after the application of test compound, MPP+ was added to a final concentration of 4 μM, diluted in control medium still in presence of compound/inhibitor for 48 h.

Test compound was tested on one culture in 96-well plate (n=6 culture wells per condition).

### End Point Evaluation:

Immunostaining: TH and α-synuclein. 48 Hours after intoxication, the cell culture supernatant was removed, and the cells were fixed by a solution of 4% paraformaldehyde in PBS, pH=7.3 for 20 min at room temperature. The cells were washed twice in PBS, and then permeabilized. Non-specific sites were blocked with a solution of PBS containing 0.1% of saponin and 1% FCS for 15 min at room temperature. The cultures were incubated with: (a) monoclonal anti-Tyrosine Hydroxylase (TH) antibody produced in mouse at dilution of 1/10000 in PBS containing 1% FCS, 0.1% saponin, for 2 hours at room temperature, and (b) polyclonal anti-alpha synuclein (α-synuclein) antibody produced in rabbit at dilution of 1/200 in PBS containing 1% FCS, 0.1% saponin, for 2 h at room temperature. These antibodies were revealed with Alexa Fluor 488 goat anti-mouse IgG at the dilution 1/800 and with Alexa Fluor 568 goat anti-rabbit IgG at the dilution 1/400 in PBS containing 1% FCS, 0.1% saponin, for 1 h at room temperature.

Automatic computer analysis: For each condition, 20 pictures (representing the whole well area) were automatically taken using ImageXpress® (Molecular Devices) at 10× magnification (20 pictures, for TH and α-synuclein into TH neurons) using the same acquisition parameters. From images, analyses was directly and automatically performed by MetaXpress® (Molecular Devices).

The following read-outs were measured:
(i) Analysis of total number of TH neurons (TH positive neurons)
(ii) Total neurite network of TH positive neurons (in μm)
(iii) α-Synuclein aggregation (overlapping between TH and α-syn staining)

Compound 12 displayed neuroprotective effects in this in vitro model of PD based on MPP+ injury. It improved neuronal survival, protected the neurite network and reduced α-Synuclein aggregation in dopaminergic neurons at a concentration of 0.5 μM.

This model functions as an in vitro model of Parkinson's disease. Activity in this model strongly supports that these compounds are efficacious in the treatment of Parkinson's disease and other autophagy-related neurodegenerative diseases or conditions.

### Bioavailability

The compounds have good properties for oral dosing and readily cross the blood brain barrier. For example, in CD1 mice, Compound 9 has a maximum concentration in brain plasma of 5067 ng/ml after 30 minutes at a dose of 30 mg/kg. Compound 12 has a maximum concentration in brain plasma of 5853 ng/ml after 8 hours at a dose of 30 mg/kg.

Hence, the compounds are suitable for oral dosing making them advantageous for the treatment of various conditions and in particular for the treatment of neurodegenerative disorders.

### Demonstration of Activity in a Mouse Model of Amyotrophic Lateral Sclerosis (ALS)

Compound 12 was selected as an example compound of this invention to be evaluated in this model.

This model (run by In vivex SAS, France) uses transgenic mice overexpressing the mutant human TDP-43 gene, harboring the alanine to threonine mutation at amino acid 315, under the control of the mouse prion promoter. These so-called “Prp-TDP43 mice” develop a progressive and fatal neurodegenerative disease with pathology reminiscent of ALS from two months old, including: (i) neuromotor impairment, (ii) decrease of nerve conduction amplitude and velocity, and (iii) a high plasma NfL (neurofilament light protein) concentration. This strain recapitulates the pathological features of ALS in humans. Any substances with positive effects in this model may be useful as a new therapeutic agent for the treatment of ALS.

### Overview of the study

Animal characteristics:
Sex/Species/Strain: Prp-TDP43; Male
Age: 3 weeks old
Approximate weight at initiation of treatment: 22.0 g±2.5 g
Animal number: 6 mice per group
Animal identification: At the beginning of the study, animals were identified with a number on the tail. Each parameter was noted in the lab book.
Acclimation and clinical signs: Animals arrived on site 7 days (one week) at 3 weeks old before the experiment to allow optimal acclimation. Clinical signs and mortality were recorded daily. Individual body weight was determined once a week during the full study.

### Study Outline:

Dose: 30 mg/kg, once per day
Administration: Oral gavage
Vehicle used for test Compound 12: 10% DMSO and 90% of 10% (2-Hydroxypropyl)-β-cyclodextrin (HP-β-CD) in water (v/v) (10%“DMSO”/90%“10% HP-β-CD” in water)
Vehicle control group: vehicle only (non-treated control)
Experimental time duration: 2 months (60 days)

### Readouts:

i) Rotarod (neuromuscular performance)
ii) Sciatic nerve electrophysiology (EMG)
iii) Plasma NfL analysis by ELISA

### Readout Schedule:

Baseline rotarod and EMG analysis at 1 month old
Rotarod, EMG and plasma NfL analysis at 2 months old
Rotarod, EMG and plasma NfL analysis at 3 months old

### Operating Procedures for Study Readouts

Sciatic nerve electrophysiology. Standard electromyography was performed on mice anesthetized with ketamine/xylazine mixture. A pair of steel needle electrodes (AD Instruments, MLA1302) were placed subcutaneously along the nerve at the sciatic notch (proximal stimulation). A second pair of electrodes were placed along the tibial nerve above the ankle (distal stimulation). Supramaximal square-wave pulses, lasting 10 ms at 1 mA were delivered using a PowerLab 26T (AD Instruments). Compound muscle action potential (CMAP) was recorded from the intrinsic foot muscles using steel electrodes. Both amplitudes and latencies of CMAP were determined. The distance between the 2 sites of stimulation was measured alongside the skin surface with fully extended legs, and nerve conduction velocities (NCVs) were calculated automatically from sciatic nerve latency measurements using Excel.

Rotarod. A rotating rod apparatus (Bioseb, France) was used to measure neuromuscular coordination and balance. Mice were first given a pretraining trial to familiarize them with the rotating rod. Latency to fall was measured at a successively increased speed from 4 to 40 rpm over a 300-second max. time period. Each animal underwent 3 trials a day. For each day, values from the 3 trials were averaged for each animal, and then averaged for each group.

Plasma NfL quantification. Plasma NfL was determined using ELISA method. The tail vein was punctured using a 25 gauge needle and 400 μL of blood was collected directly on a microtube containing EDTA as anticoagulant. Samples were centrifuged for 15 minutes at 1000×g (or 3000 rpm) at 2-8° C. within 30 minutes of collection. 200 μL of supernatant (plasma) was stored at −20° C. before ELISA analysis. NfL quantification was performed in duplicated for each animal in 96 well plates by ELISA method (Novus Biologica Ref. NBP2-80299).

### Summary of In Vivo Study Phase

### 1 Month Old Analysis (Baseline):

### Day 1: From 8 AM to 11 AM

Progressive behavioral test learning (rotarod) was carried out. In order to reduce animal stress and anxiety due to the new environment, animals remained during 2 hours in the behavioral test room.

### Day 2: From 8 AM to 11 AM—Rotarod

Neuromuscular performances was analyzed using rotarod as stated before. For each method, the values from the 3 trials were averaged for each animal, and then averaged for each treated group.

### Day 2: From 2 PM to 5 PM—Electrophysiology (EMG):

The animals were anesthetized using ketamine/xylazine mixture and sciatic nerve electrophysiology recording was performed. The CMAP was recorded from the intrinsic foot muscles using steel electrodes. Then, the NCVs of each animal were calculated from sciatic nerve latency measurements as stated before.

### From Day 3 to day 33: at 7 AM

Compound 12 was administrated by oral gavage once a day to the members of one group of mice (compound treated group). Vehicle only was administrated by oral gavage twice a day to the members of another group of mice (vehicle control group).

### 2 Month Old Analysis:

### Day 32: From 8 AM to 11 AM

Progressive behavioral test learning (rotarod) was carried out. In order to reduce animal stress and anxiety due to the new environment, animals remained during 2 hours in the behavioral test room.

### Day 33:

### From 8 AM to 11 AM—Rotarod:

Neuromuscular performances were analyzed using rotarod as stated before. For each method, the values from the 3 trials were averaged for each animal, and then averaged for each treated group.

From 2 PM to 5 PM—Electrophysiology (EMG) and blood sampling for NfL analysis: The animals were anesthetized using ketamine/xylazine mixture and sciatic nerve electrophysiology recording were performed. The CMAP was recorded from the intrinsic foot muscles using steel electrodes. Then, the NCVs of each animal were calculated from sciatic nerve latency measurements as stated before.

After EMG analysis, and in anesthetized animals, blood sampling was performed by vein tail puncture. Blood was centrifuged and plasma was stored until ELISA NfL analysis as stated before.

### From Day 34 to day 60: At 7 AM

Compound 12 was administrated by oral gavage once a day to the members of one group of mice (compound treated group). Vehicle only was administrated by oral gavage twice a day to the members of another group of mice (vehicle control group).

### 3 Month Old Analysis:

### Day 60: From 8 AM to 11 AM

Progressive behavioral test learning (rotarod) was carried out. In order to reduce animal stress and anxiety due to the new environment, animals remained during 2 hours in the behavioral test room

### Day 61:

### From 8 AM to 11 AM—Rotarod:

Neuromuscular performances were analyzed using rotarod as stated before. For each method, the values from the 3 trials were averaged for each animal, and then averaged for each treated group.

From 2 PM to 5 PM—Electrophysiology (EMG) and blood sampling for NfL analysis: The animals were anesthetized using ketamine/xylazine mixture and sciatic nerve electrophysiology recording was performed. The CMAP was recorded from the intrinsic foot muscles using steel electrodes. Then, the NCVs of each animal were calculated from sciatic nerve latency measurements as stated before.

After EMG analysis, and in anesthetized animals, blood sampling was performed by vein tail puncture. Blood was centrifuged and plasma was stored until ELISA NfL analysis as stated before.

### Terminal Procedure, Sciatic Nerve Sampling:

After electrophysiology recording and blood sampling, mice were sacrificed by cervical dislocation. Left sciatic nerve sampling was performed. Nerves were fixed using PFA 4% overnight at 4 degrees C. and then stored in fresh PBS+sodium azide 0.1% solution until histological analysis.

### Results

### Mortality and Body Weight

No mortality was observed during the study.
No pathological clinical sign was observed during the study suggesting the absence of general toxicology of Compound 12 at these experimental conditions.
As expected, a loss of body weight was observed in the ALS group treated with vehicle. An increase of the body weight was observed in the group treated with Compound 12 at 30 mg/kg compared to the vehicle group suggesting a positive effect of Compound 12 on animal body mass.

### Rotarod Test

Similar rotarod latencies were observed in between all groups at the baseline (1 month old). As expected, neuromotor impairment, characterized by a decrease of the rotarod latency was observed at two and three months old in the ALS+vehicle treated group. In mice of two months of age and three months of age, an increase of the rotaraod latency was observed in the Compound 12 treated group (at 30 mg/kg once per day) compared to the ALS+vehicle control group (an increase in mean rotarod latency of 76% at two months compared to the vehicle controlled group, and an increase in mean rotarod latency 99% at three months compared to the vehicle controlled group). These data suggest a positive effect of Compound 12 on the neuromotor performances of ALS mice when administrated once a day at 30 mg/kg.

### Sciatic Nerve Electrophysiology

### Compound Muscle Action Potential (CMAP) Amplitude

Similar CMAP amplitudes were observed in the vehicle and Compound 12 group at the baseline (1 month old). As expected, nerve conduction impairment, characterized by a decrease of the CMAP amplitude was observed at two and three months old in the ALS+vehicle treated group. In mice of two months of age and three months of age, an increase of the CMAP amplitude was observed in the Compound 12 treated group (at 30 mg/kg once per day) compared to the ALS+vehicle group (an increase in mean CAMP amplitude of 39% at two months compared to the vehicle controlled group, and an increase in mean CAMP amplitude of 73% at three months compared to the vehicle controlled group). These data suggest a positive effect of Compound 12 on sciatic nerve electrophysiology performances. Moreover, because the nerve conduction amplitude is directly linked to the axonal functionality, these results suggest that Compound 12 could target motor axons of the spinal cord and/or the peripheral nervous system.

### Nerve Conduction Velocity (NCV)

Similar nerve conduction velocities were observed in between all groups at the baseline (one month old). As expected, nerve conduction impairment, characterized by a decrease of the NCV was observed at two and three months old in the ALS+vehicle treated group. In mice of three months of age an increase of the NCV was observed in the Compound 12 treated group (at 30 mg/kg once per day) compared to the ALS+vehicle group, suggesting a positive effect of Compound 12 on the sciatic nerve electrophysiology performance at this time point (an increase in mean NCV of 139% compared to the vehicle controlled group). Moreover, because the nerve conduction velocity is directly linked to the myelin integrity, these data suggest that Compound 12 could also directly or indirectly target myelin of the spinal cord and/or the peripheral nervous system with efficacy from 3 months old.

### Plasma NfL Quantification

NfL has been reported to be a reproducible and robust biomarker for ALS disorder in rodent and humans (Verde et al. 2021). For this reason, NfL was quantified in plasma using ELISA method at two and three months old mice. In mice of two months of age and three months of age, a decrease of the plasma NfL concentration was observed in the Compound 12 treated group (at 30 mg/kg once per day) compared to ALS+vehicle group (a decrease in mean NfL concentration of 48% at two months compared to the vehicle controlled group, and a decrease in mean NfL concentration of 54% at three months compared to the vehicle controlled group). This confirms the protective molecular efficacy of Compound 12 at this dose from a biomarker point of view. Moreover, because an increase in plasma NfL is directly linked to the presence of axonal neuropathy, these data suggest a protective role of Compound 12 in the axonal degeneration induced by the ALS neuropathy.

### Summary and Conclusions

As expected, neuromotor impairment, decrease of nerve conduction amplitude and velocity and high plasma NfL concentration was observed in the preclinical ALS mouse model treated with vehicle.

The Compound 12 treated group presented an increase of the rotarod latency and nerve conduction improvement compared to the vehicle group suggesting a positive preventive efficacy of Compound 12 in ALS disorder. Moreover, a decrease of plasma NfL concentration was observed in the animals treated with Compound 12 compared to the vehicle group, which confirms the positive activity of Compound 12 from a biomarker point of view.

Taken together, this study confirms that Compound 12 has a protective effect on ALS neuropathy by increasing neuromotor and electrophysiological performances and decreasing neuropathy biomarker when administrated once a day at 30 mg/kg from one to three months old in ALS mice.

This model functions as an in vivo model of Amyotrophic Lateral Sclerosis (ALS). Activity (positive effects) in this model strongly supports that these compounds are efficacious in the treatment of Amyotrophic Lateral Sclerosis (ALS) and other autophagy-related neurodegenerative diseases or conditions.

### Demonstration of Activity in a Mouse Model of Parkinson's Disease (PD)

Compounds 12, 23 and 30 were selected as example compounds of this invention to be evaluated in this model.

This model (run by Neuro-Sys SAS, France) uses C57BL/6JRj mice injured by intra-nigral injections of a solution containing protofibrils of alpha-synuclein (α-syn, precisely quantified by automatic Western Blot) and combined with pharmacological inhibition of GBA by conduritol B epoxide (CBE), in aged mice. The model mimics the main pathologies of PD: loss of dopaminergic neurons in the substantia nigra, neuroinflammation, deficits in motor coordination and synucleinopathy (α-syn aggregation). Any substance with positive effects in this model may be useful as a new therapeutic agent for the treatment or prevention of PD.

### Overview of the Study

### Animal Characteristics:

Type of animal: C57BL/6JRj (Male)
Age: 18 months old
Supplier: Janvier Labs
Animal number: 10 mice per group
Acclimation and clinical signs: Animals arrived on site 7 days (one week) before the experiment to allow optimal acclimation.

### Dosing Outline:

Overview: CBE was administered along with the test compounds, starting on the day of the stereotaxic injections of the α-syn oligomer preparation.

### Test Compounds Dose and Administration:

Test compounds at specific concentrations (see below) were administered per os (gavage or drinking water).
Test compound treatment: The test compounds were solubilized (vehicle=water), aliquoted and stored at −20° C. Aliquots were thawed overnight at 4° C. and given once a day. Treatment started on the day of the stereotaxic injections of α-syn preparation (Day 0). Treatment was administrated until the last day of the experiment (Day 28; =day of dissection).
CBE dose and administration: 50 mg/kg, Intraperitoneal (i.p.), twice a day.
CBE treatment: Administration of CBE (vehicle=saline [NaCl 0.9%] started on the day of the stereotaxic injections of α-syn preparation (Day 0). Treatment was administrated until the last day of the experiment (Day 28; =day of dissection),
Untreated control: vehicle (saline; i.p.) administered twice a day,
Stereotaxic injections of α-syn oligomers: Human α-syn peptide (stock at 69 μM in water at −20° C.) was reconstituted at 50 μM in NaCl, 0.9% (final concentration). All mice in compound treated groups were subjected to surgery and received 2.5 μL of α-syn solution. Mice were anesthetized by isoflurane (4%, for induction), in an induction chamber coupled with a vaporizer and to an oxygen concentrator. Mice were placed on the stereotaxic frame. Anesthesia was maintained by isoflurane (2%) with a face mask coupled to the isoflurane vaporizer and oxygen concentrator machine. The skull was exposed and holes were drilled. The α-syn preparation was bilaterally injected into the SNpc, at the following coordinates: A-P, −0.3 mm; M-L, +0.12 mm; D-V, −0.45 mm. Depth of anesthesia and rectal temperature were verified every 5 minutes. After surgery, mice were allowed to recover before being placed back in the cage.

### Readouts Included:

i) Body mass
ii) Assessment of motor coordination: bar test and grid walking test
iii) Dopaminergic Tyrosine hydroxylase (TH)-positive neuron survival in the substantia nigra pars compacta (SNpc), i.e. a measure of the number of TH neurons in the SNpc
iv) α-syn area in TH positive neurons (a measure of the degree of aggregation of α-syn in TH positive neurons, in the SNpc)

### Endpoint Evaluation

Overall: For overall evaluation and body mass, mice were observed daily, and body mass was monitored prior to drug administration.
Assessment of motor coordination: bar test.

In the third week (Week 3) post-surgery, motor coordination of the mice was assessed with the bar test. The principle of the test is based on the capacity of the mice to cross a horizontal bar connected to a platform (18 mm of diameter, 60 cm long). The test requires good coordination between front and hindlimbs, and good balance. Mice are placed at one extremity of the bar and need to reach a platform located at the other side of the bar. Motor coordination was studied after two training sessions, one and two days before the test. Three sessions were recorded by a video camera system. The time to cross the bar, the number of steps and the number of failed strides was recorded.
Assessment of motor coordination: grid walking test.

A few days before the dissection (week 4), motor coordination of the mice was assessed with the horizontal grid test. The principle of the test is based on the ability of the mice to walk on a horizontal grid (30×45 cm and square: 2.5 cm). The test requires good coordination between front and hindlimbs, and good balance. Mice are placed at one end of the grid and must walk along the grid to the other end (total distance 45 cm) and let for one minute. Three sessions were recorded by a by a video camera system. The number of missed steps, which provides an indication of the extent of motor dysfunction, was recorded.

Plasma sampling, tissue collection and immunostaining.

At the end of the experiment (week 4 post-surgery), a total of 7 mice per group were deeply anesthetized. Mice were perfused with cold PBS (3 minutes), and cold paraformaldehyde (PFA) 4% in PBS (3 minutes). Immunostaining was performed with at least n=7/group. Brains were dissected and further fixed in PFA 4%, overnight at 4° C. After, brains were placed in 30% sucrose in Tris-phosphate saline (TBS) solution at 4° C. Coronal sections, including the SNpc, of 40 μm-thickness were cut using a freezing microtome (4 sections per mouse, each 100 mm apart). For immunostaining, free-floating sections were incubated in TBS with 0.25% bovine serum albumin, 0.3% Triton X-100 and 1% goat serum, for 1 hour at room temperature. This incubation blocked unspecific binding sites and permeabilized the tissues. Four (n=4) brain sections per animal were processed and incubated for 24 hours at 4° C. or 2 hours at room temperature with selected antibodies:
TH: Chicken polyclonal antibody anti-tyrosine hydroxylase (1/1000).
α-syn: Rabbit polyclonal antibody anti-alpha synuclein (1/200).

These antibodies were revealed with Alexa Fluor 488 anti-rabbit IgG, Alexa Fluor 568 anti-chicken IgG, at the dilution 1/500, incubated in TBS with 0.25% donkey serum albumin, 0.3% Triton X-100 and 1% goat serum.

Images were acquired with a confocal laser-scanning microscopy.

### Results

None of the compounds had notable effects on the body masses of the animals after 4 weeks of treatment, suggesting the absence of a systemic toxic effect of the compounds at the analyzed time points.

Compound 12: A Dose of 30 mg/Kg Per Os Once a Day

In the bar test, the following results were obtained: (i) a 21% decrease in the mean time required to cross the bar in the compound treated group compared to the α-syn/CBE injured group, (ii) a 2% reduction in the mean number of steps required to cross the bar in the compound treated group compared to the α-syn/CBE injured group, and (iii) a 33% reduction in the number of misplaced steps (failed strides) in the compound treated group compared to the α-syn/CBE injured group. In the grid walking test, the following results were obtained: (i) a 21% reduction in the mean number of failures per meter (number of missed steps), and (ii) a 12% increase in the total distance travelled during the test. A 36% increase in the number of dopaminergic TH positive neurons in the SNpc was observed in the compound treated group compared to the α-syn/CBE injured group. In addition, a 10% decrease in the aggregation of α-syn in TH neurons (α-syn area) was observed in the compound treated group compared to the α-syn/CBE injured group. Taken together, this data indicates that Compound 12 at the stated dose was able to reduce some of the essential neuropathological features of PD that are reproduced in this model, and also reduce some of the deficits in motor coordination related to PD that are reproduced in this model.

Compound 23: A Dose of 30 mg/Kg Dose Per Os on the First Day of Treatment, Followed by a 10 mg/Kg Dose Per Os Once a Day Thereafter

In the bar test, the following results were obtained: (i) an 11% decrease in the mean time required to cross the bar in the compound treated group compared to the α-syn/CBE injured group, (ii) a 3% reduction in the mean number of steps required to cross the bar in the compound treated group compared to the α-syn/CBE injured group, and (iii) a 39% reduction in the number of misplaced steps (failed strides) in the compound treated group compared to the α-syn/CBE injured group. In the grid walking test, the following results were obtained: (i) a 14% reduction in the mean number of failures per meter (number of missed steps), and (ii) a 14% increase in the total distance travelled during the test. A 31% increase in the number of dopaminergic TH positive neurons in the SNpc was observed in the compound treated group compared to the α-syn/CBE injured group. In addition, a 16% decrease in the aggregation of α-syn in TH neurons (α-syn area) was observed in the compound treated group compared to the α-syn/CBE injured group. Taken together, this data indicates that Compound 23 at the stated dose was able to reduce some of the essential neuropathological features of PD that are reproduced in this model, and also reduce some of the deficits in motor coordination related to PD that are reproduced in this model.

Compound 30: A Dose of 30 mg/Kg Dose Per Os on the First Day of Treatment, Followed by a 10 mg/Kg Dose Per Os Once a Day Thereafter

In the bar test, the following results were obtained: (i) a 25% decrease in the mean time required to cross the bar in the compound treated group compared to the α-syn/CBE injured group, (ii) a 7% reduction in the mean number of steps required to cross the bar in the compound treated group compared to the α-syn/CBE injured group, and (iii) a 19% reduction in the number of misplaced steps (failed strides) in the compound treated group compared to the α-syn/CBE injured group. In the grid walking test, the following results were obtained: (i) a 28% reduction in the mean number of failures per meter (number of missed steps), and (ii) a 36% increase in the total distance travelled during the test. A 57% increase in the number of dopaminergic TH positive neurons in the SNpc was observed in the compound treated group compared to the α-syn/CBE injured group. In addition, a 30% decrease in the aggregation of α-syn in TH neurons (α-syn area) was observed in the compound treated group compared to the α-syn/CBE injured group. Taken together, this data indicates that Compound 30 at the stated dose was able to reduce some of the essential neuropathological features of PD that are reproduced in this model, and also reduce some of the deficits in motor coordination related to PD that are reproduced in this model.

This model functions as an in vivo model of Parkinson's disease. Activity in this model (positive effects) strongly support that these compounds are efficacious in the treatment of Parkinson's disease and other autophagy-related neurodegenerative diseases or conditions.

### REFERENCES

Callizot N, Combes M, Henriques A, Poindron P. Necrosis, apoptosis, necroptosis, three modes of action of dopaminergic neuron neurotoxins. PLoS ONE 14(4): e0215277
Dauer W., Przedborski S. Parkinson's disease: mechanisms and models. Neuron 2003, 39(6):889-909
Visanji N P, Orsi A, Johnston T H, Howson P A, Dixon K, Callizot N, Brotchie J M and Rees D D. PYM50028, a novel, orally active, nonpeptide neurotrophic factor inducer, prevents and reverses neuronal damage induced by MPP+ in mesencephalic neurons and by MPTP in a mouse model of Parkinson's disease. FASEB J. 2008; 22(7):2488-97
Verde F, Otto M, Silani V. Neurofilament Light Chain as Biomarker for Amyotrophic Lateral Sclerosis and Frontotemporal Dementia. Frontiers in Neuroscience. 2021; 15:679199 (doi: 10.3389/fnins.2021.679199)
Zhang X J, Chen S, Huang K X, Le W D. Why should autophagic flux be assessed?Acta Pharmacol Sin. 2013 May; 34(5): 595-9. doi: 10.1038/aps.2012.184. Epub 2013 Mar. 11

In particular, the present invention relates to the following items.
1. A compound according to formula (I)

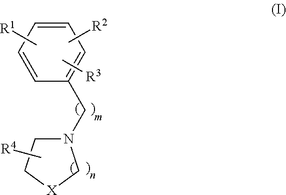

or a salt, stereoisomer, tautomer or N-oxide thereof,
wherein
X is CH₂, CHR⁴, NR^{N} or O;
R¹ is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X};
R² is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X};
R³ is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X}; or
R¹ and R² together with the atoms to which they are attached form a 5- or 6-membered partially unsaturated heterocyclyl, wherein the aforementioned heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{X};
R⁴ is H, C₁-C₄-alkyl, or —(CH₂)ₚ—NR^{a}R^{b};
wherein
R^{N} is H, or C₁-C₄-alkyl;
R^{X} is C₁-C₄-alkyl, C₁-C₄-haloalkyl, NR^{a}R^{b}, or two R^{X} form cyclopropyl;
R^{a}, R^{b} are independently of each other selected from H, and C₁-C₄-alkyl;
R^{c} is H, or C₁-C₄-alkyl;
m is 1, 2, or 3;
n is 0, 1, or 2;
p is 0, 1, 2, or 3.
2. The compound according to item 1, wherein the compound is not

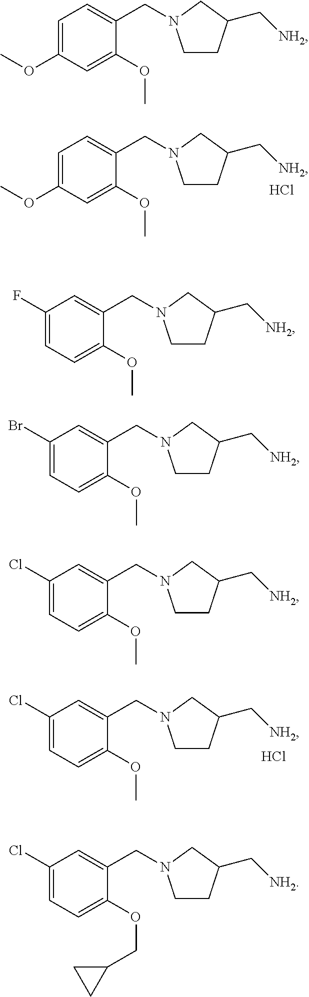

3. The compound according to item 1 or 2, wherein
R¹ is C₁-C₄-alkoxy, wherein each substitutable carbon atom in the aforementioned groups is independently unsubstituted or substituted with one or more, same or different substituents R^{X};
R² is halogen;
R³ is H or halogen; and
R⁴ is —(CH₂)ₚ—NR^{a}R^{b}.
4. The compound according to any one of items 1 to 3, wherein
R¹ is C₂-C₄-alkoxy, wherein each substitutable carbon atom in the aforementioned groups is independently unsubstituted or substituted with one or more, same or different substituents R^{X};
R² is Cl;
R³ is H or Cl; and
R⁴ is —(CH₂)ₚ—NR^{a}R^{b}.
5. The compound according to any one of items 1 to 4, wherein the compound according to formula (I) is a compound according to the following formula

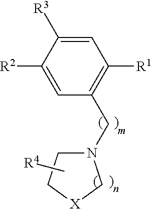

wherein
R¹ is C₂-C₄-alkoxy, wherein each substitutable carbon atom in the aforementioned groups is independently unsubstituted or substituted with one or more, same or different substituents R^{X};
R² is Cl;
R³ is H or Cl; and
R⁴ is —(CH₂)ₚ—NR^{a}R^{b}.
6. The compound according to any one of items 1 to 5, wherein
X is CH₂, or CHR⁴.
7. The compound according to any one of items 1 to 6, wherein
m is 1;
n is 0, 1 or 2, preferably 1 or 2; and
p is 0 or 1, preferably 1.
8. The compound according to any one of items 1 to 7, wherein the compound of formula (I) is present in the form of a salt, preferably in the form of a hydrochloride salt.
9. The compound according to any one of items 1 to 8, wherein the compound of formula (I) is selected from the group consisting of (S)-1-(2-ethoxy-5-(trifluoromethyl)benzyl)-3-methylpiperazine hydrochloride, (S)-(1-(2-ethoxy-5-(trifluoromethyl) benzyl) pyrrolidin-3-yl) methanamine hydrochloride, (S)-(1-(5-chloro-2-propoxybenzyl) pyrrolidin-3-yl) methanamine Hydrochloride, (S)-(1-(4,5-dichloro-2-ethoxybenzyl)pyrrolidin-3-yl)methanamine hydrochloride, (S)-(1-(4,5-dichloro-2-(cyclopropylmethoxy)benzyl)pyrrolidin-3-yl)methanamine hydrochloride, (S)-(1-(5-chloro-2-ethoxybenzyl)piperidin-3-yl)methanamine hydrochloride, (S)-(1-(5-chloro-2-(cyclopropylmethoxy)benzyl)piperidin-3-yl)methanamine hydrochloride, (S)-1-(5-chloro-2-ethoxybenzyl)-3-methylpiperazine hydrochloride, 1-(5-chloro-2-ethoxybenzyl)-4-ethylpiperazine, 1-(5-chloro-2-ethoxybenzyl)-N,N-dimethylpiperidin-4-amine, (S)-(1-(5-chloro-2-ethoxybenzyl)pyrrolidin-3-yl)methanamine hydrochloride, (S)-(1-(5-chloro-2-ethoxybenzyl)pyrrolidin-2-yl)methanamine Hydrochloride, (R)-(1-(5-chloro-2-ethoxybenzyl)pyrrolidin-2-yl)methanamine hydrochloride, (R)-1-(5-chloro-2-ethoxybenzyl)piperidin-3-amine hydrochloride, (S)-1-(5-chloro-2-ethoxybenzyl)piperidin-3-amine hydrochloride, (1-(5-chloro-2-ethoxybenzyl)azetidin-3-yl)methanamine Hydrochloride, and 1-(5-chloro-2-ethoxybenzyl)-4-isopropylpiperazine.
10. A pharmaceutical composition comprising a pharmaceutically effective amount of the compound according to any one of items 1 to 9 and optionally a pharmaceutically acceptable carrier, diluent or excipient.
11. A compound according to any one of items 1 to 9 or a pharmaceutical composition according to item 10 for use in medicine.
12. A compound according to any one of items 1 to 9 or a pharmaceutical composition according to item 10 for use in the treatment of an autophagy-related disease or condition.
13. The compound or the pharmaceutical composition for use according to item 12, wherein said autophagy-related disease or condition is selected from the group consisting of neurodegenerative diseases, Huntington's disease, Alzheimer's disease, Parkinson's disease, systemic lupus erythematosus, epilepsy, cancer, liver diseases (e.g. nonalcoholic fatty liver disease (NAFLD)), al antitrypsin deficiency, Charcot Marie Tooth syndrome, Rett Syndrome, Sickle Cell disease, Wilson Disease, amyloidosis, Gaucher's diseases, lysosomal and glycogen storage disorders (e.g., Glycogen Storage Disease type 1A (GSD1A)), cystic fibrosis; viral infection and diseases human cytomegalovirus (HCMV) infection, hepatitis B, human immunodeficiency virus infection, Zika virus infection, coronavirus infection, HCoV-229E, HCoV-NL63, betacoronavirus infection, such as HCoV-OC43, SARS-CoV-1, HCoV-HKU1, MERS-CoV or SARS-CoV-2, bacterial infections, metabolic disorders, diabetes, fibrosis, silicosis, diabetic retinopathy, glaucoma, cataracts, age-related macular degeneration, glomerulonephritis, glomerulosclerosis, wound healing disorders, Niemann-Pick type C (NPC) disease, fibrinogen storage disease (FSB), inclusion body disease (IBD), muscular dystrophy, Duchenne muscular dystrophy, Limb-girdle muscular dystrophy, myopathy, myofibrillar myopathy, hereditary myopathy, diabetic cardiomyopathy, anti-inflammatory disorders, autoimmune diseases, multiple sclerosis, rheumatoid arthritis, irritable bowel syndrome, Crohn's disease, vascular disorders, stroke, coronary artery diseases, myocardial infarction, unstable angina pectoris, atherosclerosis or vasculitis, Behcet's syndrome, giant cell arteritis, polymyalgia rheumatic, Wegener's granulomatosis, Churg-Strauss syndrome, vasculitis, Henoch-Schonlein pruprua, Kawasaki disease, viral infection or replication, pox virus infection, herpes virus infection, asthma, allergic rhinitis, COPD, osteoporosis, organ transplant rejection, psoriasis, hypertrophic scarring (keloid formation), adhesion formations following general or gynecological surgery, lung fibrosis, liver fibrosis, kidney fibrosis, disorders caused by intracellular parasites, malaria, tuberculosis, neuropathic pain, post-operative phantom limb pain or postherpetic neuralgia, allergies, amyotrophic lateral sclerosis (ALS), antigen induced recall response, immune response suppression, muscle degeneration and atrophy, frailty in aging, spinal cord injury, and diseases and conditions involving misfolded and/or nonfolded proteins.
14. The compound or the pharmaceutical composition for use according to item 12 or 13, wherein said treatment comprises a combination of at least one compound according to any one of items 1 to 9 with at least one additional pharmaceutically active substance for said autophagy-related disease or condition.
15. Use of a compound according to any one of items 1 to 9 for stimulating autophagy in an in-vitro assay.

1. A compound according to formula (I) 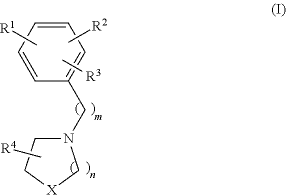
or a salt, stereoisomer, tautomer or N-oxide thereof,
wherein
X is CH₂, CHR⁴, NR^{N} or O;
R¹ is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X};
R² is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X};
R³ is H, CN, halogen, C₁-C₄-alkyl, C₁-C₄-haloalkyl, C₁-C₄-alkoxy, C₁-C₄-haloalkoxy, or —O—C₁-C₄-alkylene-OR^{c}, wherein each substitutable carbon atom in the aforementioned groups is independently unsubstituted or substituted with one or more, same or different substituents R^{X}; or
R¹ and R² together with the atoms to which they are attached form a 5- or 6-membered partially unsaturated heterocyclyl, wherein the aforementioned heterocyclic ring comprises one or more, same or different heteroatoms selected from O, N and S, wherein said N- and/or S-atoms are independently oxidized or non-oxidized, and wherein each substitutable carbon or heteroatom in the aforementioned groups is independently unsubstituted or substituted with one or more, same or different substituents R^{X};
R⁴ is H, C₁-C₄-alkyl, or —(CH₂)ₚ—NR^{a}R^{b};
wherein
R^{N} is H, or C₁-C₄-alkyl;
R^{X} is C₁-C₄-alkyl, C₁-C₄-haloalkyl, NR^{a}R^{b}, or two R^{X} form cyclopropyl;
R^{a}, R^{b} are independently of each other selected from H, and C₁-C₄-alkyl;
R^{c} is H, or C₁-C₄-alkyl;
m is 1, 2, or 3;
n is 0, 1, or 2;
p is 0, 1, 2, or 3.

2. The compound according to claim 1, wherein the compound is not

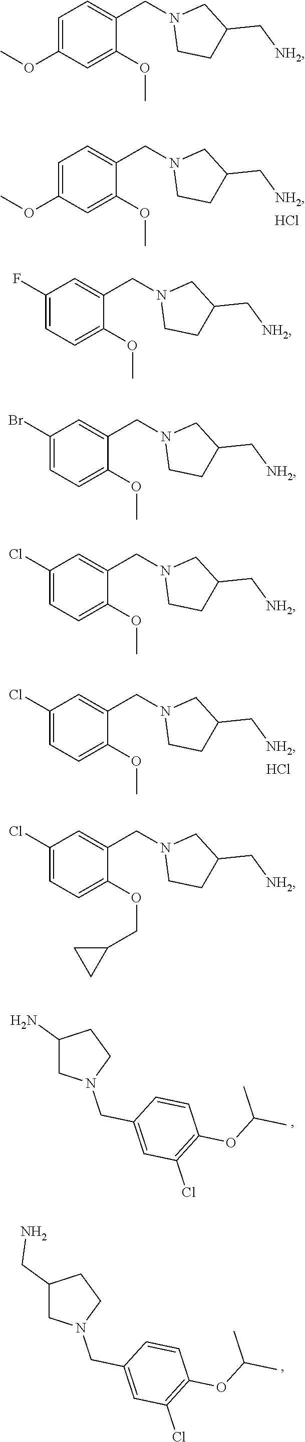

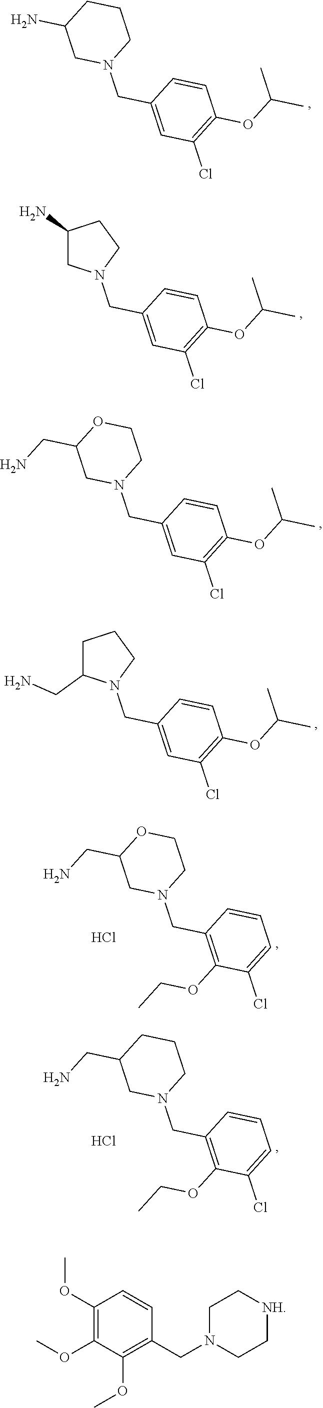

3. The compound according to claim 1, wherein
R¹ is C₁-C₄-alkoxy, wherein each substitutable carbon atom in the aforementioned groups is independently unsubstituted or substituted with one or more, same or different substituents R^{X};
R² is halogen;
R³ is H or halogen; and
R⁴ is —(CH₂)ₚ—NR^{a}R^{b}.

4. The compound according to claim 1, wherein
R¹ is C₂-C₄-alkoxy, wherein each substitutable carbon atom in the aforementioned groups is independently unsubstituted or substituted with one or more, same or different substituents R^{X};
R² is Cl;
R³ is H or Cl; and
R⁴ is —(CH₂)ₚ—NR^{a}R^{b}.

5. The compound according to claim 1, wherein the compound according to formula (I) is a compound according to the following formula 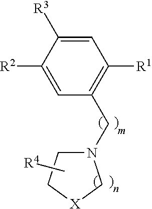
wherein
R¹ is C₂-C₄-alkoxy, wherein each substitutable carbon atom in the aforementioned groups is independently unsubstituted or substituted with one or more, same or different substituents R^{X};
R² is Cl;
R³ is H or Cl; and
R⁴ is —(CH₂)ₚ—NR^{a}R^{b}.

6. The compound according to claim 1, wherein
X is CH₂, or CHR⁴.

7. The compound according to claim 1, wherein
m is 1;
n is 0, 1 or 2, preferably 1 or 2; and
p is 0 or 1, preferably 1.

8. The compound according to claim 1, wherein the compound of formula (I) is present in the form of a salt, preferably in the form of a hydrochloride salt.

9. The compound according to claim 1, wherein the compound of formula (I) is selected from the group consisting of (S)-1-(2-ethoxy-5-(trifluoromethyl)benzyl)-3-methylpiperazine hydrochloride, (S)-(1-(2-ethoxy-5-(trifluoromethyl) benzyl) pyrrolidin-3-yl) methanamine hydrochloride, (S)-(1-(5-chloro-2-propoxybenzyl) pyrrolidin-3-yl) methanamine Hydrochloride, (S)-(1-(4,5-dichloro-2-ethoxybenzyl)pyrrolidin-3-yl)methanamine hydrochloride, (S)-(1-(4,5-dichloro-2-(cyclopropylmethoxy)benzyl)pyrrolidin-3-yl)methanamine hydrochloride, (S)-(1-(5-chloro-2-ethoxybenzyl)piperidin-3-yl)methanamine hydrochloride, (S)-(1-(5-chloro-2-(cyclopropylmethoxy)benzyl)piperidin-3-yl)methanamine hydrochloride, (S)-1-(5-chloro-2-ethoxybenzyl)-3-methylpiperazine hydrochloride, 1-(5-chloro-2-ethoxybenzyl)-4-ethylpiperazine, 1-(5-chloro-2-ethoxybenzyl)-N,N-dimethylpiperidin-4-amine, (S)-(1-(5-chloro-2-ethoxybenzyl)pyrrolidin-3-yl)methanamine hydrochloride, (S)-(1-(5-chloro-2-ethoxybenzyl)pyrrolidin-2-yl)methanamine Hydrochloride, (R)-(1-(5-chloro-2-ethoxybenzyl)pyrrolidin-2-yl)methanamine hydrochloride, (R)-1-(5-chloro-2-ethoxybenzyl)piperidin-3-amine hydrochloride, (S)-1-(5-chloro-2-ethoxybenzyl)piperidin-3-amine hydrochloride, (1-(5-chloro-2-ethoxybenzyl)azetidin-3-yl)methanamine Hydrochloride, and 1-(5-chloro-2-ethoxybenzyl)-4-isopropylpiperazine.

10. The compound according to claim 1, wherein the compound of formula (I) is selected from the group consisting of
(S)-(1-(3,5-dichloro-4-ethoxybenzyl)pyrrolidin-3-yl)methanamine hydrochloride, 1-[2-(3-chloro-4-ethoxyphenyl)ethyl]-4-piperidylamine hydrochloride,
(S)-1-{2-[3,5-dichloro-4-(2-methoxyethoxy)phenyl]ethyl}-3-piperidylamine hydrochloride,
(R)-1-{[3,5-dichloro-4-(cyclopropylmethoxy)phenyl]methyl}-3-pyrrolidinylamine hydrochloride,
(S)-1-{2-[3,5-dichloro-4-(cyclopropylmethoxy)phenyl]ethyl}-3-piperidylamine hydrochloride, 1-[(3-chloro-4-ethoxyphenyl)methyl]-4-piperidylamine hydrochloride,
(R)-1-{2-[3,5-dichloro-4-(2-methoxyethoxy)phenyl]ethyl}-3-piperidylamine hydrochloride,
(S)-1-{[3,5-dichloro-4-(cyclopropylmethoxy)phenyl]methyl}-3-pyrrolidinylamine hydrochloride,
(R)-1-{2-[3,5-dichloro-4-(cyclopropylmethoxy)phenyl]ethyl}-3-piperidylamine hydrochloride,
(S)-1-{2-[3-chloro-4-(cyclopropylmethoxy)phenyl]ethyl}-3-piperidylamine formate,
(S)-1-{2-[3-chloro-4-(cyclopropylmethoxy)phenyl]ethyl}-3-piperidylamine hydrochloride,
(R)-1-{2-[3-chloro-4-(cyclopropylmethoxy)phenyl]ethyl}-3-piperidylamine formate,
(R)-1-{2-[3-chloro-4-(cyclopropylmethoxy)phenyl]ethyl}-3-piperidylamine hydrochloride,
1-{[3-chloro-4-(cyclopropylmethoxy)phenyl]methyl}-4-piperidylamine hydrochloride,
5-{[(R)-3-(Aminomethyl)-1-pyrrolidinyl]methyl}-3-chloro-2-ethoxybenzonitrile hydrochloride,
3-{2-[(S)-3-Amino-1-piperidyl]ethyl}-4-ethoxybenzonitrile hydrochloride,
3-{2-[(R)-3-Amino-1-piperidyl]ethyl}-4-ethoxybenzonitrile hydrochloride,
5-{2-[(S)-3-amino-1-pyrrolidinyl]ethyl}-2-ethoxybenzonitrile hydrochloride,
3-{2-[(S)-3-Amino-1-pyrrolidinyl]ethyl}-4-ethoxybenzonitrile hydrochloride,
3-{2-[(R)-3-amino-1-pyrrolidinyl]ethyl}-4-ethoxybenzonitrile hydrochloride,
(R)-1-[2-(4-ethoxy-3-fluorophenyl)ethyl]-3-pyrrolidinylamine hydrochloride,
(R)-1-{2-[3-chloro-4-(2-methoxyethoxy)phenyl]ethyl}-3-piperidylamine hydrochloride,
1-[(3,5-Dichloro-4-ethoxyphenyl)methyl]-4-piperidylamine hydrochloride,
1-{2-[3,5-Dichloro-4-(cyclopropylmethoxy)phenyl]ethyl}-4-piperidylamine hydrochloride,
(S)-1-[(3-chloro-4-ethoxyphenyl)methyl]-3-piperidylamine hydrochloride,
(R)-1-[(3-chloro-4-ethoxyphenyl)methyl]-3-piperidylamine hydrochloride,
1-{[3,5-dichloro-4-(2-methoxyethoxy)phenyl]methyl}-4-piperidylamine hydrochloride,
1-{[3-chloro-4-(2-methoxyethoxy)phenyl]methyl}-4-piperidylamine hydrochloride,
(S)-1-{[3,5-dichloro-4-(cyclopropylmethoxy)phenyl]methyl}-3-piperidylamine hydrochloride,
(R)-1-{[3,5-dichloro-4-(cyclopropylmethoxy)phenyl]methyl}-3-piperidylamine hydrochloride,
1-{[3,5-dichloro-4-(cyclopropylmethoxy)phenyl]methyl}-4-piperidylamine hydrochloride,
(R)-1-{2-[3-chloro-4-(cyclopropylmethoxy)phenyl]ethyl}-3-pyrrolidinylamine hydrochloride,
(S)-1-{2-[3-chloro-4-(cyclopropylmethoxy)phenyl]ethyl}-3-pyrrolidinylamine hydrochloride,
({(S)-1-[(3-chloro-5-ethoxyphenyl)methyl]-3-pyrrolidinyl}methyl)amine hydrochloride, and
({1-[(5-chloro-2-ethoxyphenyl)methyl]-3-azetidinyl}methyl)amine hydrochloride.

11. A pharmaceutical composition comprising a pharmaceutically effective amount of the compound according to claim 1 and optionally a pharmaceutically acceptable carrier, diluent or excipient.

12. A method for stimulating autophagy, comprising contacting cells with a compound according to claim 1 or a pharmaceutical composition comprising the compound to stimulate autophagy in the cells.

13. A method for treating an autophagy-related disease or condition, comprising administering to a subject in need thereof an effective amount of a compound according to claim 1 or a pharmaceutical composition comprising the compound.

14. The method according to claim 13, wherein said autophagy-related disease or condition is selected from the group consisting of neurodegenerative diseases, Huntington's disease, Alzheimer's disease, Parkinson's disease, systemic lupus erythematosus, epilepsy, cancer, liver diseases (e.g. nonalcoholic fatty liver disease (NAFLD)), al antitrypsin deficiency, Charcot Marie Tooth syndrome, Rett Syndrome, Sickle Cell disease, Wilson Disease, amyloidosis, Gaucher's diseases, lysosomal and glycogen storage disorders (e.g., Glycogen Storage Disease type 1A (GSD1A)), cystic fibrosis; viral infection and diseases human cytomegalovirus (HCMV) infection, hepatitis B, human immunodeficiency virus infection, Zika virus infection, coronavirus infection, HCoV-229E, HCoV-NL63, betacoronavirus infection, such as HCoV-OC43, SARS-CoV-1, HCoV-HKU1, MERS-CoV or SARS-CoV-2, bacterial infections, metabolic disorders, diabetes, fibrosis, silicosis, diabetic retinopathy, glaucoma, cataracts, age-related macular degeneration, glomerulonephritis, glomerulosclerosis, wound healing disorders, Niemann-Pick type C (NPC) disease, fibrinogen storage disease (FSB), inclusion body disease (IBD), muscular dystrophy, Duchenne muscular dystrophy, Limb-girdle muscular dystrophy, myopathy, myofibrillar myopathy, hereditary myopathy, diabetic cardiomyopathy, anti-inflammatory disorders, autoimmune diseases, multiple sclerosis, rheumatoid arthritis, irritable bowel syndrome, Crohn's disease, vascular disorders, stroke, coronary artery diseases, myocardial infarction, unstable angina pectoris, atherosclerosis or vasculitis, Behcet's syndrome, giant cell arteritis, polymyalgia rheumatic, Wegener's granulomatosis, Churg-Strauss syndrome, vasculitis, Henoch-Schonlein pruprua, Kawasaki disease, viral infection or replication, pox virus infection, herpes virus infection, asthma, allergic rhinitis, COPD, osteoporosis, organ transplant rejection, psoriasis, hypertrophic scarring (keloid formation), adhesion formations following general or gynecological surgery, lung fibrosis, liver fibrosis, kidney fibrosis, disorders caused by intracellular parasites, malaria, tuberculosis, neuropathic pain, post-operative phantom limb pain or postherpetic neuralgia, allergies, amyotrophic lateral sclerosis (ALS), antigen induced recall response, immune response suppression, muscle degeneration and atrophy, frailty in aging, spinal cord injury, and diseases and conditions involving misfolded and/or nonfolded proteins.

15. The method of claim 13, further comprising administering to the subject at least one additional pharmaceutically active substance for said autophagy-related disease or condition.

16. The method of claim 12, wherein the method is an in-vitro assay.
